(19) United States
(12) Patent Application Publication
(10) Pub. No.: US 2024/0390522 A1
(43) Pub. Date: Nov. 28, 2024

(54) GENE THERAPY FOR DUCHENNE MUSCULAR DYSTROPHY

(71) Applicant: University of Florida Research Foundation, Incorporated, Gainesville, FL (US)

(72) Inventor: Hugh Lee Sweeney, Gainesville, FL (US)

(73) Assignee: University of Florida Research Foundation, Incorporated, Gainesville, FL (US)

(21) Appl. No.: 18/696,085

(22) PCT Filed: Sep. 28, 2022
(86) PCT No.: PCT/US2022/077199

§ 371 Date: Mar. 27, 2024

Related U.S. Application Data
(60) Provisional application No. 63/303,499, filed on Jan. 26, 2022.
(60) Provisional application No. 63/249,511, filed on Sep. 28, 2021.

(51) Int. Cl. A61K 48/00; A61P 21/00; C07K 14/47; C12N 15/86

(52) U.S. Cl. CPC A61K 48/0058; A61P 21/00; C07K 14/4708; C12N 15/86; C12N 2750/14143; C12N 2830/008

(57) ABSTRACT

The disclosure provides compositions and methods for producing micro-dystrophin proteins, and the use thereof (e.g., in gene therapy). The disclosure also provides skeletal muscle-specific promoters and the use thereof (e.g., in gene therapy). The disclosure further provides catheter-based methods of delivering gene therapy vectors to skeletal muscle and/or cardiac muscle.

### CROSS-REFERENCE TO RELATED APPLICATIONS

This application claims the benefit of the filing dates of U.S. Provisional Application No. 63/249,511, filed Sep. 28, 2021 and U.S. Provisional Application No. 63/303,499, filed Jan. 26, 2022, the entire contents of each of which are incorporated by reference.

### BACKGROUND

Duchenne muscular dystrophy (DMD) is a rare, X chromosome-linked disorder characterized by progressive muscle degeneration and weakness. DMD is caused by mutations in the gene encoding the dystrophin protein, which is the largest known gene. The full-length dystrophin protein, as expressed in skeletal muscle, smooth muscle, and cardiomyocytes, is 3685 amino acids and has a molecular weight of 427 kD. The severe Duchenne phenotype is generally associated with the loss of full-length dystrophin protein from skeletal and cardiac muscle, which leads to debilitating muscle degeneration and, ultimately, heart failure. A large number of different dystrophin gene mutations have been described, many of them resulting in either the severe DMD or the milder Becker Muscular Dystrophy.

Utrophin protein is highly related to dystrophin and can substitute for dystrophin's function in mammals. Normally, utrophin is expressed highly in developing muscle, and in mature muscle is enriched at the neuromuscular junction. As the myofiber matures, utrophin levels are decreased and dystrophin is the protein expressed in the mature myofiber.

The multifaceted pathologies associated with DMD represent major challenges to the development and clinical implementation of effective therapeutics for the disease. There are several therapeutic strategies being pursued for the treatment of DMD. The most active area of research is in “gene replacement” strategies (see Oshima et al. (2009) J. Am. Soc. Gene Ther. 17:73-80; Liu et al. (2005) Mol. Ther. 11:245-56; Lai et al. (2006) Hum Gene Ther. 17:1036-42; Odom et al. (2008) Mol. Ther. 16:1539-45). This approach involves delivering a functional copy of the dystrophin gene to patients using a viral delivery vector, typically adeno-associated virus (AAV), which can deliver a micro-dystrophin construct to all striated muscles. The large size of the dystrophin gene makes it incompatible with the limited carrying capacity of common viral vectors, however. This necessitates the use of a “micro-dystrophin” gene, in which most of the repetitive central portion of the gene is removed to leave only the minimally functional protein. It is not clear, however, that expression of “micro-dystrophin” is sufficient for clinical benefit. In addition, this approach suffers from the possibility of random gene integration into the patient genome, which could lead to insertional mutagenesis, and the potential for immune reactions against the delivery vector. Thus, a number of significant drawbacks remain with micro-dystrophin gene therapies. Accordingly, a need exists for alternative gene therapies for treating DMD.

### SUMMARY

The present disclosure provides chimeric proteins that comprise domains from dystrophin and utrophin proteins. These chimeric proteins are useful for treating subjects suffering from skeletal muscle disorders such as DMD. The present disclosure also provides recombinant nucleic acids encoding any of these chimeric proteins. In addition, this disclosure provides rAAV vectors comprising any of these nucleic acids, and rAAV particles comprising these rAAV vectors. In some aspects, the present disclosure provides cells and compositions comprising any of the disclosed chimeric proteins, recombinant nucleic acids, rAAV vectors, or rAAV particles. In some aspects, the disclosure provides methods of treatment of DMD and other skeletal muscle disorders.

Accordingly, in some aspects, the present disclosure provides a recombinant nucleic acid comprising a nucleotide sequence encoding a chimeric protein comprising a dystrophin region (or domain) and a utrophin region (or domain). In some embodiments, the chimeric protein comprises a carboxy-terminal (also referred to herein as “C-terminal” or “C-terminus”) region of dystrophin coupled to an amino-terminal (also referred to herein as “N-terminal” or “N-terminus”) region of utrophin. In one aspect, the present disclosure provides a recombinant nucleic acid comprising a nucleotide sequence encoding a micro-dystrophin protein comprising a utrophin N-terminus. In some embodiments, the micro-dystrophin protein comprises the region of utrophin from the N-terminus to spectrin-like repeat 1. In some embodiments, the micro-dystrophin protein comprises the region of utrophin from the N-terminus to spectrin-like repeat 2. In some embodiments, the micro-dystrophin protein comprises the region of utrophin from the N-terminus to spectrin-like repeat 3. In some embodiments, the micro-dystrophin protein comprises the region of utrophin from the N-terminus to hinge 2.

In some embodiments, the recombinant nucleic acid comprises a micro-dystrophin protein-encoding nucleotide sequence that is at least 80% identical to the nucleotide sequence of any one of SEQ ID NOs: 7-12 and 22-39. In some embodiments, this recombinant nucleic acid comprises a nucleotide sequence having 100% identity with the nucleotide sequence of any one of SEQ ID NOs: 7-12 and 22-39. In some embodiments, the recombinant nucleic acid comprises a nucleotide sequence encoding the micro-dystrophin protein that comprises a nucleotide sequence that is at least 80% identical to the nucleotide sequence of any one of SEQ ID Nos. 7-12 and 22-39.

In some embodiments, the recombinant nucleic acid comprises a nucleotide sequence that is at least 80% identical to the nucleotide sequence of any one of SEQ ID NOs: 85-92, 126-131, and 133-134. In some embodiments, the recombinant nucleic acid comprises a nucleotide sequence having 100% identity with the nucleotide sequence of any one of SEQ ID NOs: 85-92, 126-131, and 133-134. In some embodiments, the recombinant nucleic acid comprises a nucleotide sequence that is at least 80% identical to the nucleotide sequence of any one of SEQ ID NOs: 135-148. In some embodiments, the recombinant nucleic acid comprises a nucleotide sequence having 100% identity with the nucleotide sequence of any one of SEQ ID NOs: 135-148. In some embodiments, the recombinant nucleic acid comprises a nucleotide sequence that is at least 80% identical to the nucleotide sequence of any one of SEQ ID NOs: 166-185. In some embodiments, the recombinant nucleic acid comprises a nucleotide sequence having 100% identity with the nucleotide sequence of any one of SEQ ID NOs: 166-185.

In one aspect, the present disclosure provides a recombinant nucleic acid encoding a chimeric protein that comprises, or consists of, a nucleotide sequence encoding a micro-dystrophin protein comprising: an amino-terminal actin-binding domain, hinge domain 1, spectrin-like repeat 1, spectrin-like repeat 17, spectrin-like repeat 18, and spectrin-like repeat 19, and wherein the micro-dystrophin protein does not contain spectrin-like repeat 2 and/or spectrin-like repeat 3 of dystrophin. In some embodiments, the micro-dystrophin protein further comprises one or more of: spectrin-like repeat 24, hinge domain 4, and dystroglycan binding site of dystrophin. In some embodiments, the micro-dystrophin protein further comprises one or more syntrophin binding domains of dystrophin. In some embodiments, the micro-dystrophin protein further comprises one or more coiled coil domains of dystrophin. In some embodiments, the spectrin-like repeat 1 is directly coupled to the spectrin-like repeat 17. In some embodiments, the spectrin-like repeat 19 is directly coupled to spectrin-like repeat 24. In some embodiments, the spectrin-like repeat 19 is directly coupled to hinge domain 4.

In exemplary embodiments, provided herein is a chimeric protein that contains a utrophin N-terminal domain ending at utrophin spectrin-like repeat 2 connected to a dystrophin C-terminal domain containing spectrin-like repeat 23 through the C-terminus. Also provided herein is a chimeric protein that consists of a utrophin N-terminal domain ending at utrophin spectrin-like repeat 2 connected to a dystrophin C-terminal domain containing spectrin-like repeat 23 through the C-terminus.

In some aspects, the present disclosure provides a recombinant nucleic acid encoding a chimeric protein that comprises a nucleotide sequence encoding a micro-dystrophin protein comprising: an amino-terminal actin-binding domain, hinge domain 1, spectrin-like repeat 1, spectrin-like repeat 2, spectrin-like repeat 16, and spectrin-like repeat 17, and wherein the micro-dystrophin protein does not contain spectrin-like repeat 3 of dystrophin.

In some embodiments, the micro-dystrophin protein further comprises one or more of: spectrin-like repeat 23, spectrin-like repeat 24, hinge domain 4, and dystroglycan binding site of dystrophin. In some embodiments, the micro-dystrophin protein further comprises one or more syntrophin binding domains of dystrophin. In some embodiments, the micro-dystrophin protein further comprises one or more coiled coil domains of dystrophin.

In some embodiments, the spectrin-like repeat 2 is directly coupled to spectrin-like repeat 16. In some embodiments, the spectrin-like repeat 17 is directly coupled to spectrin-like repeat 24. In some embodiments, the spectrin-like repeat 17 is directly coupled to hinge domain 4.

In some embodiments, the micro-dystrophin protein of the chimeric protein comprises the region of dystrophin from spectrin-like repeat 24 to the C-terminus. In some embodiments, the micro-dystrophin protein comprises the region of dystrophin from spectrin-like repeat 24 to the end of the proline rich region following the first coiled coil domain. In some embodiments, the micro-dystrophin protein comprises the region of dystrophin from hinge domain 4 to the C-terminus.

In some embodiments, the micro-dystrophin protein of the chimeric protein comprises, or consists of, the region of dystrophin from hinge domain 4 to the end of the proline rich region following the first coiled coil domain. In some embodiments, the micro-dystrophin protein comprises the region of dystrophin from hinge domain 4 to the end of the second syntrophin binding domain.

In some embodiments, any of the disclosed recombinant nucleic acids encoding the chimeric protein is less than 5 kb in length.

In some embodiments, a promoter is operably linked to the nucleotide sequence encoding the micro-dystrophin protein. In some embodiments, the promoter is a cardiac-specific promoter. In some embodiments, the cardiac-specific promoter is a cardiac troponin T (cTnT) promoter. In some embodiments, the promoter is a skeletal muscle-specific promoter. In some embodiments, the skeletal muscle-specific promoter is a skeletal muscle alpha-actin promoter. In some embodiments, the skeletal muscle-specific promoter comprises a nucleotide sequence that is at least 80% identical to SEQ ID NO: 78 or SEQ ID NO: 80.

In some embodiments, the present disclosure provides a recombinant nucleic acid comprising a nucleotide sequence encoding a micro-dystrophin protein that comprises an amino acid sequence that is at least 80% identical to the amino acid sequence of any one of SEQ ID Nos. 1-6 and 13-21.

In various embodiments, the recombinant nucleic acid comprising a nucleotide sequence encoding a micro-dystrophin protein as described herein also comprises a nucleotide sequence encoding a region of a utrophin protein. In some embodiments, a promoter is operably linked to the nucleotide sequence encoding the region of utrophin protein. In some embodiments, the micro-dystrophin protein comprises one or more utrophin protein domains.

In some embodiments, the chimeric protein comprises an N-terminal domain (or N-terminus) containing a region of utrophin from the N-terminus to spectrin-like repeat 1; the region of utrophin from the N-terminus to spectrin-like repeat 2; the region of utrophin from the N-terminus to spectrin-like repeat 3; or the region of utrophin from the N-terminus to hinge 2. In some embodiments, the utrophin domain consists of the region of utrophin from the N-terminus to spectrin-like repeat 1; the region of utrophin from the N-terminus to spectrin-like repeat 2; the region of utrophin from the N-terminus to spectrin-like repeat 3; or the region of utrophin from the N-terminus to hinge 2.

In some embodiments, the present disclosure provides a micro-dystrophin protein encoded by a recombinant nucleic acid of the disclosure. In some embodiments, the present disclosure provides a chimeric protein comprising a micro-dystrophin protein region and a utrophin protein region, wherein the chimeric protein is encoded by a recombinant nucleic acid as provided in the disclosure.

In one aspect, the present disclosure provides a skeletal muscle-specific promoter. In one aspect, the present disclosure provides an isolated nucleic acid comprising a nucleotide sequence that is at least 80% identical to the nucleotide sequence of SEQ ID NO: 78 or SEQ ID NO: 80. In some embodiments, this nucleotide sequence is operably linked to a nucleotide sequence that is at least 80% identical to the nucleotide sequence of any one of SEQ ID Nos. 7-12 and 22-39.

In some embodiments, the nucleotide sequence that is at least 80% identical to the nucleotide sequence of SEQ ID NO: 78 or SEQ ID NO: 80 is operably linked to a nucleotide sequence that encodes a micro-dystrophin protein that comprises an amino acid sequence that is at least 80% identical to the amino acid sequence of any one of SEQ ID Nos. 1-6 and 13-21.

In one aspect, the present disclosure provides a recombinant adeno-associated virus (rAAV) vector comprising a recombinant nucleic acid or isolated nucleic acid of the disclosure.

In one aspect, the present disclosure provides an rAAV particle comprising a rAAV vector of the disclosure encapsidated in an AAV capsid. In some embodiments, the AAV capsid comprises a capsid protein derived from AAV1, AAV2, AAV3, AAV6, AAV7, AAV8, AAVrh.74, AAVrh.10, AAV2/6 or AAV9 serotypes. In one aspect, the present disclosure provides compositions comprising one or more of these rAAV particles.

In one aspect, the present disclosure provides a method of treating a skeletal muscle disorder in a subject in need thereof, comprising administering to the subject a recombinant or isolated nucleic acid of the disclosure, an rAAV particle of the disclosure, or a composition of the disclosure.

In one aspect, the present disclosure provides a method of treating Duchenne muscular dystrophy (DMD) in a subject in need thereof, comprising administering to the subject a recombinant or isolated nucleic acid of the disclosure, an rAAV particle of the disclosure, or a composition of the disclosure.

In one aspect, the present disclosure provides a method of treating Duchenne muscular dystrophy (DMD) in a subject in need thereof, the method comprising delivering to the subject a first recombinant adeno-associated virus (rAAV) particle comprising a nucleotide sequence encoding a first micro-dystrophin protein to cardiac muscle (e.g., optimized for cardiac muscle) and a second rAAV particle comprising a nucleotide sequence encoding a second micro-dystrophin to skeletal muscle (e.g., optimized for skeletal muscle). In some embodiments, the first recombinant adeno-associated virus (rAAV) particle comprising a nucleotide sequence encoding a first micro-dystrophin protein adapted for cardiac muscle also comprises a nucleotide sequence encoding a first utrophin protein region. In some embodiments, the second rAAV particle comprising a nucleotide sequence encoding a second micro-dystrophin adapted for skeletal muscle also comprises a nucleotide sequence encoding a second utrophin protein region. In some embodiments, the first recombinant adeno-associated virus (rAAV) particle comprises a nucleotide sequence encoding a first chimeric protein comprising a first micro-dystrophin protein and a first utrophin protein region to cardiac muscle. In some embodiments, the second recombinant adeno-associated virus (rAAV) particle comprises a nucleotide sequence encoding a second chimeric protein comprising a second micro-dystrophin protein and a second utrophin protein region to skeletal muscle. In some embodiments, the first or second micro-dystrophin protein comprises one or more utrophin protein domains. In some embodiments, an rAAV particle comprising a nucleotide sequence encoding a micro-dystrophin protein adapted for one type (e.g., cardiac or skeletal) of muscle tissue may be delivered to another type (e.g., cardiac or skeletal) of muscle tissue. In some embodiments, the first rAAV particle is adapted for delivery to cardiac muscle tissue. In some embodiments, the second rAAV particle is adapted for delivery to skeletal muscle tissue.

In some embodiments, methods are provided for transducing a cardiac muscle cell with the first rAAV particle of the disclosure. In some embodiments, methods are provided for transducing a skeletal muscle cell with the second rAAV particle of the disclosure.

In some embodiments, methods are provided for delivery of the first rAAV particle to cardiac muscle tissue. In some embodiments, methods are provided for delivery of the second rAAV particle to skeletal muscle tissue.

In some embodiments, the first rAAV particle comprises a cardiac-specific promoter operably linked to the nucleotide sequence encoding the first micro-dystrophin protein. In some embodiments, the second rAAV particle comprises a skeletal muscle-specific promoter operably linked to the nucleotide sequence encoding the second micro-dystrophin protein. In some embodiments, the first rAAV particle comprises a cardiac-specific promoter operably linked to the nucleotide sequence encoding the first chimeric protein. In some embodiments, the second rAAV particle comprises a skeletal muscle-specific promoter operably linked to the nucleotide sequence encoding the second chimeric protein. The first chimeric protein may comprise any of the proteins comprising a micro-dystrophin protein region and a utrophin protein region disclosed herein. The second chimeric protein may comprise any of the proteins comprising a micro-dystrophin protein region and a utrophin protein region disclosed herein. In some embodiments, the first chimeric protein and the second chimeric protein is the same. In some embodiments, the first chimeric protein and the second chimeric protein are different.

As such, provided herein are methods of treatment comprising delivering to the subject a first rAAV particle comprising a vector encoding any of the disclosed chimeric proteins to cardiac muscle and a second rAAV particle comprising a vector encoding any of the disclosed chimeric proteins to skeletal muscle. In some embodiments, these methods are intended for treatment of a subject suffering from a skeletal muscle disorder. In some embodiments, these methods are intended for treatment of a subject suffering from DMD.

In some embodiments, the first and second rAAV particles are of the same serotype. In some embodiments, the first and second rAAV particles are of different serotypes. In some embodiments, the first rAAV particle comprises a capsid protein derived from AAV9, AAVrh.74, or AAVrh.10. In some embodiments, the second rAAV particle comprises a capsid protein derived from AAV8.

In some embodiments, the first and second rAAV particles are delivered by the same route of administration. In some embodiments, the first and second rAAV particles are delivered by a single catheter. In some embodiments, the first and second rAAV particles are delivered by different routes of administration. In some embodiments, the first and second rAAV particles are delivered by different catheters. In some embodiments, the first and second rAAV particles are delivered by two or more (e.g., two, three, four, five, or more than five) catheters. Delivery of the first rAAV particle and the second rAAV particle may be relatively simultaneous, or at different times. In some embodiments, delivery of the first rAAV particle and the second rAAV particle are through different routes of administration and at different times. In some embodiments, delivery of the first rAAV particle occurs within 1-24 hours, 24-48 hours, 48-72 hours, 72 hours to a week, 1 week to 2 weeks, 1 week to 3 weeks, 2 weeks to 4 weeks, more than 4 weeks after, or more than 5 weeks after, of delivery of the second rAAV particle

In some embodiments, the first and second rAAV particles are delivered via one or more catheters. In some embodiments, the method comprises: (i) introducing a catheter into the femoral artery and advancing to the heart; (ii) delivering the first rAAV particle into the left and right coronary arteries; (iii) retracting the catheter to the aortic arch; and (iv) delivering the second rAAV particle to the subclavian and/or carotid arteries. In some embodiments, the method further comprises (v) retracting the catheter into the descending aorta and delivering the second rAAV particle to skeletal muscle via descending aortic branches.

In some aspects, the present disclosure provides a method of delivering an rAAV particle to a skeletal muscle in a subject, the method comprising delivering the rAAV particle via a catheter. In some embodiments, the method comprises delivering the rAAV particle to the subclavian and/or carotid arteries. In some embodiments, the catheter is first introduced into the femoral artery and advanced to the subclavian and/or carotid arteries. In some embodiments, the method further comprises retracting the catheter into the descending aorta and delivering the rAAV particle to skeletal muscle via descending aortic branches.

In some embodiments, the method further comprises administering a vasodilator to the subject prior to the delivery of the first and second rAAV particles. In some embodiments, a blood pressure cuff is inflated on each limb at the time of delivery of the first rAAV particle and/or the second rAAV particle. In some embodiments, the skeletal muscle is fast-twitch or slow-twitch.

In some embodiments, the vasodilator is a PDE5 inhibitor. In some embodiments, the PDE5 inhibitor is sildenafil or tadalafil. In some embodiments, the vasodilator is administered 10 minutes, 20 minutes, 30 minutes, 40 minutes, 50 minutes, or 1 hour prior to the delivery of first rAAV and/or second rAAV.

In some embodiments, the first micro-dystrophin protein comprises: an amino-terminal actin-binding domain, hinge domain 1, spectrin-like repeat 1, spectrin-like repeat 17, spectrin-like repeat 18, and spectrin-like repeat 19, and wherein the micro-dystrophin protein does not contain spectrin-like repeat 2 and/or spectrin-like repeat 3 of dystrophin. In some embodiments, the first micro-dystrophin protein further comprises one or more of: spectrin-like repeat 24, hinge domain 4, and dystroglycan binding site of dystrophin. In some embodiments, the first micro-dystrophin protein further comprises one or more syntrophin binding domains of dystrophin. In some embodiments, the first micro-dystrophin protein further comprises one or more coiled coil domains of dystrophin. In some embodiments, the spectrin-like repeat 1 of the first micro-dystrophin protein is directly coupled to the spectrin-like repeat 17. In some embodiments, the spectrin-like repeat 19 of the first micro-dystrophin protein is directly coupled to spectrin-like repeat 24. In some embodiments, the spectrin-like repeat 19 of the first micro-dystrophin protein is directly coupled to hinge domain 4.

In some embodiments, the second micro-dystrophin protein comprises: an amino-terminal actin-binding domain, hinge domain 1, spectrin-like repeat 1, spectrin-like repeat 2, spectrin-like repeat 16, and spectrin-like repeat 17, and wherein the micro-dystrophin protein does not contain spectrin-like repeat 3 of dystrophin. In some embodiments, the second micro-dystrophin protein further comprises one or more of: spectrin-like repeat 23, spectrin-like repeat 24, hinge domain 4, and dystroglycan binding site of dystrophin. In some embodiments, the second micro-dystrophin protein further comprises one or more syntrophin binding domains of dystrophin. In some embodiments, the second micro-dystrophin protein further comprises one or more coiled coil domains of dystrophin. In some embodiments, the spectrin-like repeat 2 of the second micro-dystrophin protein is directly coupled to spectrin-like repeat 16. In some embodiments, the spectrin-like repeat 17 of the second micro-dystrophin protein is directly coupled to spectrin-like repeat 24. In some embodiments, the spectrin-like repeat 17 of the second micro-dystrophin protein is directly coupled to hinge domain 4.

In some embodiments, the first or second micro-dystrophin protein comprises the region of dystrophin from spectrin-like repeat 24 to the C-terminus. In some embodiments, the first or second micro-dystrophin protein comprises the region of dystrophin from spectrin-like repeat 24 to the end of the proline rich region following the first coiled coil domain. In some embodiments, the first or second micro-dystrophin protein comprises the region of dystrophin from hinge domain 4 to the C-terminus. In some embodiments, the first or second micro-dystrophin protein comprises the region of dystrophin from hinge domain 4 to the end of the proline rich region following the first coiled coil domain. In some embodiments, the first or second micro-dystrophin protein comprises the region of dystrophin from hinge domain 4 to the end of the second syntrophin binding domain. In some embodiments, the first or second micro-dystrophin protein comprises the region of utrophin from the N-terminus to spectrin-like repeat 1. In some embodiments, the first or second micro-dystrophin protein comprises the region of utrophin from the N-terminus to spectrin-like repeat 2. In some embodiments, the first or second micro-dystrophin protein comprises the region of utrophin from the N-terminus to spectrin-like repeat 3. In some embodiments, the first or second micro-dystrophin protein comprises the region of utrophin from the N-terminus to hinge 2.

### BRIEF DESCRIPTION OF DRAWINGS

FIG. 1 shows a diagram of dystrophin and its interactions, showing known syntrophin binding sites (or domains).

FIG. 2 shows a diagram of full-length dystrophin showing order of Repeats (R) and hinges (H), as well as the cysteine-rich portion of the C-terminus (CR), which is commonly included in micro-dystrophins and referred to as the truncated C-terminus, which is then followed by the rest of the C-terminus (CT).

FIG. 3 shows the structure of dystrophin and micro-dystrophin constructs. A schematic diagram of full-length dystrophin and the micro-dystrophin versions that are being evaluated in current clinical trials. Also diagrammed is the variant of the ΔR3-R19/ΔR20-R21 ΔCT micro-dystrophin construct (ΔR3-R21 ΔCT), which was compared to exemplary micro-utrophin/dystrophin chimeric proteins of the disclosure. In ΔR3-R21 ΔCT, the N terminus of utrophin (ending at utrophin repeat 2) is connected to dystrophin repeat 23 through the C-terminal syntrophin binding sites of dystrophin.

FIG. 4 shows the results of protection from contraction-induced injury resulting from an exemplary micro-utrophin/dystrophin chimeric protein of the disclosure. These experiments compared the loss of creatine kinase from muscle fibers due to membrane leakage and loss of force generation due to muscle damage following administration of the chimeric protein and several micro-dystrophin constructs Published protocols were used to compare the degree of protection conferred by the chimeric protein to that conferred by the micro-dystrophins shown in FIG. 3, similar to those used in clinical trials. SPₒ, specific force.

FIG. 5 shows how the chimera contains the N terminus of utrophin, ending at utrophin repeat 2, to dystrophin repeat 23, through the C-terminal syntrophin binding sites of dystrophin.

FIG. 6 shows the loss of force generation due to eccentric muscle damage. Note that it is possible for AAV-delivery of the utrophin/dystrophin chimera to provide as much protection as delivery of the micro-dystrophin construct, and protects more than delivering neither construct.

FIG. 7 shows the maintained force generation in dystrophic muscle. Both the maximal absolute force generation and the specific force (SPₒ) production, measured by force per cross-sectional area, are reduced by the loss of dystrophin. AAV-delivery of either the micro-dystrophin or the micro-utrophin/dystrophin chimera may improve the force towards normal. Con=control.

### DETAILED DESCRIPTION

The following detailed description is made by way of illustration of certain aspects of the disclosure. It is to be understood that other aspects are contemplated and may be made without departing from the scope or spirit of the present disclosure. The following detailed description, therefore, is not to be taken in a limiting sense. Scientific and technical terms used herein have meanings commonly used in the art unless otherwise specified. The definitions provided herein are to facilitate understanding of certain terms used frequently herein and are not meant to limit the scope of the present disclosure.

### Overview

The present disclosure relates to compositions and methods of cardiac muscle and/or skeletal muscle gene therapy, e.g., for treating DMD.

Gene therapy for DMD has moved into the clinic, with four ongoing clinical trials and more planned. The ongoing trials deliver a truncated version of the deficient protein, dystrophin, to heart and skeletal muscle using AAV. The reason for delivery of a truncated protein, known as micro-dystrophin is that the full length protein will not fit within the packaging limits of AAV.

However, a number of significant problems remain. There is a need to prevent and resolve fibrosis in order to improve the efficiency of delivery and enable the use of lower viral doses. Additional therapeutics may be needed to slow the skeletal muscle disease progression that remains after micro-dystrophin therapy. Perhaps the most critical issue facing AAV gene therapy for DMD is that current micro-dystrophin designs and vector doses are optimized for skeletal muscle without sufficient evaluation of the potential impact on cardiac muscle function.

The micro-dystrophins in trials have resulted from more than a decade of investigation of constructs in the dystrophin-deficient mdx mouse. The inventors have recently analyzed these constructs in clinical trials in a newer, more severe mouse model (D2.mdx mouse) that is much more indicative of the human disease. Without wishing to be bound by theory, based on the results of these mouse studies, it is predicted that the long-term consequences of existing gene therapies will be a form of DMD with severe cardiomyopathy and slowly failing skeletal muscle function. Accordingly, the inventors have identified the need for a different approach to micro-dystrophin delivery to skeletal muscle versus to the heart.

Designing different micro-dystrophin constructs that are optimal for each tissue will significantly improve the therapeutic effects of gene therapy with micro-dystrophin. In some embodiments, this involves simultaneous delivery of two separate vectors, one delivered systemically to skeletal muscles and one delivered directly into the coronary arteries of the heart, using different micro-dystrophin constructs and doses. In some embodiments, a small, skeletal muscle (fast and slow)-specific promoter is used for skeletal muscle. In some embodiments, a cardiac-specific promoter is used for the heart. The disclosure provides micro-dystrophin constructs with improved cardiac-corrective properties, likely to be safer and more beneficial than those in current trials. By combining additional gene therapy approaches with micro-dystrophin, long-lasting benefits to both striated muscle types can be achieved.

Accordingly, in one aspect, the present disclosure provides compositions and methods for producing micro-dystrophin proteins, and the use thereof. The present disclosure provides nucleic acids encoding micro-dystrophin proteins optimized for either skeletal muscle or cardiac muscle. As used herein in the context of a target tissue, the phrase “optimized for” refers to a construct that contains elements that are more beneficial for the target tissue (either cardiac or skeletal muscle). A construct that is “optimized for” a target tissue may however be expressed in, and beneficial for, tissues other than the target tissue (e.g., both skeletal and cardiac muscle). In some embodiments, the nucleic acids encoding micro-dystrophin proteins are optimized for cardiac muscle. In some embodiments, the nucleic acids encoding micro-dystrophin proteins are optimized for skeletal muscle. In some embodiments, the present disclosure provides compositions and methods for producing chimeric proteins comprising a micro-dystrophin protein as described herein and a utrophin protein region, and the use thereof.

In another aspect, the present disclosure relates to skeletal muscle-specific promoters and the use thereof.

In yet another aspect, the present disclosure relates to localized delivery (e.g., catheter-based delivery) of gene therapy vectors (e.g., rAAV vectors) to skeletal muscle and/or cardiac muscle. The inventors have found that catheter-based delivery of gene therapy vectors to skeletal muscle and/or cardiac muscle is more efficient and allows for a better distribution of the gene therapy vectors, enabling the use of lower doses of the gene therapy vectors. This in turn mitigates any unfavorable immune responses to the gene therapy vectors. In some embodiments, the present disclosure provides a catheter-based method of delivering a gene therapy vector (e.g., an rAAV vector) to skeletal muscle. In some embodiments, the present disclosure provides a catheter-based method of simultaneously delivering one or more gene therapy vectors (e.g., one or more rAAV vectors) to skeletal muscle and cardiac muscle. In some embodiments, the gene therapy vector targeted to skeletal muscle comprises a nucleic acid encoding a micro-dystrophin protein that is optimized for skeletal muscle and the gene therapy vector targeted to cardiac muscle comprises a nucleic acid encoding a micro-dystrophin protein that is optimized for cardiac muscle.

### Chimeric Proteins and Constructs

The micro-dystrophin constructs disclosed herein may encode a chimeric protein with one or more heterologous domains (e.g., one or more utrophin domains). In some aspects, the present disclosure provides a chimeric construct comprising a nucleotide sequence encoding a micro-dystrophin protein comprising one or more utrophin protein domain. In some aspects, the present disclosure provides a chimeric protein comprising a dystrophin region (or domain) and a utrophin region (or domain). In some embodiments, the present disclosure provides a recombinant nucleic acid comprising a nucleotide sequence encoding a chimeric protein comprising a dystrophin region and a utrophin region. In some embodiments, the chimeric protein comprises a carboxy-terminal (also referred to herein as “C-terminal” or “C-terminus”) region of dystrophin coupled to an amino-terminal (also referred to herein as “N-terminal” or “N-terminus”) region of utrophin. In some embodiments, the chimeric protein comprises one or more N-terminal regions of utrophin. In some embodiments, the chimeric protein consists of N-terminal regions of utrophin coupled to the C-terminal regions of dystrophin.

A micro-dystrophin gene as used herein refers to a truncated dystrophin gene that is generally less than 5 kb in length. A micro-dystrophin gene is generally small enough to fit into an AAV vector. In some embodiments, a micro-dystrophin gene is less than about 4900 bp in length. In some embodiments, a micro-dystrophin gene is less than about 4800 bp in length. In some embodiments, a micro-dystrophin gene is less than about 4700, 4600, or 4500 bp in length. In some embodiments, a micro-dystrophin gene is between about 3600-5000 bp in length. In some embodiments, a micro-dystrophin gene is between about 3600-4800 bp in length, about 3600-4500 bp in length, about 3300-5000 bp in length, about 3300 to 4800 bp in length, about 3300 to 4500 bp in length, about 3000-5000 bp in length, about 3000-4800 bp in length, or about 3000-4500 bp in length. A micro-dystrophin protein as used herein is less than about 1700 aa in length. In some embodiments, a micro-dystrophin protein is less than about 1666 aa in length. In some embodiments, a micro-dystrophin protein is less than about 1600 aa in length. In some embodiments, a micro-dystrophin protein is between about 1200-1700 aa in length. In some embodiments, a micro-dystrophin protein is between about 1200-1666 aa in length, about 1200-1600 aa in length, about 1100-1700 aa in length, about 1100-1666 aa in length, about 1100 to 1600 aa in length, about 1100-1700 aa in length, about 1000-1666 aa in length, or about 1000-1600 aa in length. A micro-dystrophin gene may include naturally occurring dystrophin sequences from any species and variants derived from such genes by mutagenesis, or other modifications.

In some embodiments, a micro-dystrophin protein as described herein further comprises one or more utrophin protein domains. In some embodiments, a micro-dystrophin protein optimized for cardiac muscle comprises a utrophin N-terminus. In some embodiments, a micro-dystrophin protein comprises the region of utrophin from the N-terminus to spectrin-like repeat 1.

Non-limiting examples of human chimeric utrophin/dystrophin micro-dystrophin proteins optimized for cardiac muscle are described below.

Utrophin N-terminus through R1_Dystrophin R17-R19_R24+H4+trCterm:
(SEQ ID NO: 81)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMAASARSVEKWRRFHYDIKIFNQWLTEAEQF
LRKTQIPENWEHAKYKWYLKELQDGIGQRQTVVRTLNATGEEIIQQSSKTDASILQEKLGSLNLRWQEVCKQLSD
RKKRLEEQKNILSEFQRDLNEFVLWLEEADNIASIPLEPGKEQQLKEKLEQVKLLVEELPLRQGILKQLNETGGP
VLVSAPISPEEQDKLENKLKQTNLQWIKVSRALPEKQGEIEAQIKDLGQLEKKLEDLEEQLNHLLLWLSPIRNQL
EIYNQPNQEGPFDVKETEIAVQAKQPDVEEILSKGQHLYKEKPATQPVKRKLEDLSSEWKAVNRLLQELRAKQWQ
RKIDETLERLQELQEATDELDLKLRQAEVIKGSWQPVGDLLIDSLQDHLEKVKALRGEIAPLKENVSHVNDLARQ
LTTLGIQLSPYNLSTLEDLNTRWKLLQVAVEDRVRQLHEAHRDFGPASQHELSTSVQGPWERAISPNKVPYYINH
ETQTTCWDHPKMTELYQSLADLNNVRFSAYRTAMKLRRLQKALCLDLLSLSAACDALDQHNLKQNDQPMDILQII
NCLTTIYDRLEQEHNNLVNVPLCVDMCLNWLLNVYDTGRIGRIRVLSFKTGIISLCKAHLEDKYRYLFKQVASST
GFCDQRRLGLLLHDSIQIPRQLGEVASFGGSNIEPSVRSCFQFANNKPEIEAALFLDWMRLEPQSMVWLPVLHRV
AAAETAKHQAKCNICKECPIIGFRYRSLKHFNYDICQSCFFSGRVAKGHKMHYPMVEYCTPTTSGEDVRDFAKVL
KNKFRTKRYFAKHPRMGYLPVQTVLEGDNMETP
Utrophin N-terminus through R1_Dystrophin R17-R19_H4 to 1st coiled
coil and proline rich region that follows:
(SEQ ID NO: 82)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMAASARSVEKWRRFHYDIKIFNQWLTEAEQF
LRKTQIPENWEHAKYKWYLKELQDGIGQRQTVVRTLNATGEEIIQQSSKTDASILQEKLGSLNLRWQEVCKQLSD
RKKRLEEQKNILSEFQRDLNEFVLWLEEADNIASIPLEPGKEQQLKEKLEQVKLLVEELPLRQGILKQLNETGGP
VLVSAPISPEEQDKLENKLKQTNLQWIKVSRALPEKQGEIEAQIKDLGQLEKKLEDLEEQLNHLLLWLSPIRNQL
EIYNQPNQEGPFDVKETEIAVQAKQPDVEEILSKGQHLYKEKPATQPVKRKLEDLSSEWKAVNRLLQELRAKQGP
ASQHELSTSVQGPWERAISPNKVPYYINHETQTTCWDHPKMTELYQSLADLNNVRESAYRTAMKLRRLQKALCLD
LLSLSAACDALDQHNLKQNDQPMDILQIINCLTTIYDRLEQEHNNLVNVPLCVDMCLNWLLNVYDTGRTGRIRVL
SFKTGIISLCKAHLEDKYRYLFKQVASSTGFCDQRRLGLLLHDSIQIPRQLGEVASEGGSNIEPSVRSCFQFANN
KPEIEAALFLDWMRLEPQSMVWLPVLHRVAAAETAKHQAKCNICKECPIIGFRYRSLKHENYDICQSCFFSGRVA
KGHKMHYPMVEYCTPTTSGEDVRDFAKVLKNKFRTKRYFAKHPRMGYLPVQTVLEGDNMETPVTLINFWPVDSAP
ASSPQLSHDDTHSRIEHYASRLAEMENSNGSYLNDSISPNESIDDEHLLIQHYCQSLNQDSPLSQPRSPAQILIS
LESEERGELERILADLEEENRNLQAEYDRLKQQHEHKGLSPLPSPPEMMPTSPQSP
Utrophin N-terminus through R1_Dystrophin R17-R19_R24 to end of
syntrophin region:
(SEQ ID NO: 83)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMAASARSVEKWRRFHYDIKIFNQWLTEAEQF
LRKTQIPENWEHAKYKWYLKELQDGIGQRQTVVRTLNATGEEIIQQSSKTDASILQEKLGSLNLRWQEVCKQLSD
RKKRLEEQKNILSEFQRDLNEFVLWLEEADNIASIPLEPGKEQQLKEKLEQVKLLVEELPLRQGILKQLNETGGP
VLVSAPISPEEQDKLENKLKQTNLQWIKVSRALPEKQGEIEAQIKDLGQLEKKLEDLEEQLNHLLLWLSPIRNQL
EIYNQPNQEGPFDVKETEIAVQAKQPDVEEILSKGQHLYKEKPATQPVKRKLEDLSSEWKAVNRLLQELRAKQWQ
RKIDETLERLQELQEATDELDLKLRQAEVIKGSWQPVGDLLIDSLQDHLEKVKALRGEIAPLKENVSHVNDLARQ
LTTLGIQLSPYNLSTLEDLNTRWKLLQVAVEDRVRQLHEAHRDFGPASQHELSTSVQGPWERAISPNKVPYYINH
ETQTTCWDHPKMTELYQSLADLNNVRESAYRTAMKLRRLQKALCLDLLSLSAACDALDQHNLKQNDQPMDILQII
NCLTTIYDRLEQEHNNLVNVPLCVDMCLNWLLNVYDTGRIGRIRVLSFKTGIISLCKAHLEDKYRYLFKQVASST
GFCDQRRLGLLLHDSIQIPRQLGEVASFGGSNIEPSVRSCFQFANNKPEIEAALFLDWMRLEPQSMVWLPVLHRV
AAAETAKHQAKCNICKECPIIGFRYRSLKHFNYDICQSCFFSGRVAKGHKMHYPMVEYCTPTTSGEDVRDFAKVL
KNKFRTKRYFAKHPRMGYLPVQTVLEGDNMETPVTLINFWPVDSAPASSPQLSHDDTHSRIEHYASRLAEMENSN
GSYLNDSISPNESIDDEHLLIQHYCQSLNQDSPLSQPRS
Utrophin N-terminus through R1_Dystrophin R23 to end of dystrophin:
(SEQ ID NO: 84)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMAASASSDQWKRLHLSLQELLVWLQLKDDEL
SRQAPIGGDFPAVQKQNDVHRAFKRELKTKEPVIMSTLETVRIFLTEQPLEGLEKLYQEPRELPPEERAQNVTRL
LRKQAEEVNTEWEKLNLHSADWQRKIDETLERLQELQEATDELDLKLRQAEVIKGSWQPVGDLLIDSLQDHLEKV
KALRGEIAPLKENVSHVNDLARQLTTLGIQLSPYNLSTLEDLNTRWKLLQVAVEDRVRQLHEAHRDFGPASQHEL
STSVQGPWERAISPNKVPYYINHETQTTCWDHPKMTELYQSLADLNNVRESAYRTAMKLRRLQKALCLDLLSLSA
ACDALDQHNLKQNDQPMDILQIINCLTTIYDRLEQEHNNLVNVPLCVDMCLNWLLNVYDTGRIGRIRVLSFKTGI
ISLCKAHLEDKYRYLFKQVASSTGFCDQRRLGLLLHDSIQIPRQLGEVASFGGSNIEPSVRSCFQFANNKPEIEA
ALFLDWMRLEPQSMVWLPVLHRVAAAETAKHQAKCNICKECPIIGFRYRSLKHFNYDICQSCFFSGRVAKGHKMH
YPMVEYCTPTTSGEDVRDFAKVLKNKFRTKRYFAKHPRMGYLPVQTVLEGDNMETPVTLINFWPVDSAPASSPQL
SHDDTHSRIEHYASRLAEMENSNGSYLNDSISPNESIDDEHLLIQHYCQSLNQDSPLSQPRSPAQILISLESEER
GELERILADLEEENRNLQAEYDRLKQQHEHKGLSPLPSPPEMMPTSPQSPRDAELIAEAKLLRQHKGRLEARMQI
LEDHNKQLESQLHRLRQLLEQPQAEAKVNGTTVSSPSTSLQRSDSSQPMLLRVVGSQTSDSMGEEDLLSPPQDTS
TGLEEVMEQLNNSFPSSRGRNTPGKPMREDTM

### Nucleic Acid Sequences Encoding Chimeric Proteins

Non-limiting examples of nucleotide sequences encoding human chimeric utrophin-dystrophin micro-dystrophin proteins optimized for cardiac muscle are described below.

Utrophin N-terminus through R1_Dystrophin R17-R19_R24+H4+trCterm:
Human Sequences without codon alterations:
(SEQ ID NO: 85)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGCCGCTAGCGCCAGA
TCTGTTGAGAAATGGCGGCGTTTTCATTATGATATAAAGATATTTAATCAGTGGCTAACAGAAGCTGAACAGTTT
CTCAGAAAGACACAAATTCCTGAGAATTGGGAACATGCTAAATACAAATGGTATCTTAAGGAACTCCAGGATGGC
ATTGGGCAGCGGCAAACTGTTGTCAGAACATTGAATGCAACTGGGGAAGAAATAATTCAGCAATCCTCAAAAACA
GATGCCAGTATTCTACAGGAAAAATTGGGAAGCCTGAATCTGCGGTGGCAGGAGGTCTGCAAACAGCTGTCAGAC
AGAAAAAAGAGGCTAGAAGAACAAAAGAATATCTTGTCAGAATTTCAAAGAGATTTAAATGAATTTGTTTTATGG
TTGGAGGAAGCAGATAACATTGCTAGTATCCCACTTGAACCTGGAAAAGAGCAGCAACTAAAAGAAAAGCTTGAG
CAAGTCAAGTTACTGGTGGAAGAGTTGCCCCTGCGCCAGGGAATTCTCAAACAATTAAATGAAACTGGAGGACCC
GTGCTTGTAAGTGCTCCCATAAGCCCAGAAGAGCAAGATAAACTTGAAAATAAGCTCAAGCAGACAAATCTCCAG
TGGATAAAGGTTTCCAGAGCTTTACCTGAGAAACAAGGAGAAATTGAAGCTCAAATAAAAGACCTTGGGCAGCTT
GAAAAAAAGCTTGAAGACCTTGAAGAGCAGTTAAATCATCTGCTGCTGTGGTTATCTCCTATTAGGAATCAGTTG
GAAATTTATAACCAACCAAACCAAGAAGGACCATTTGACGTTAAGGAAACTGAAATAGCAGTTCAAGCTAAACAA
CCGGATGTGGAAGAGATTTTGTCTAAAGGGCAGCATTTGTACAAGGAAAAACCAGCCACTCAGCCAGTGAAGAGG
AAGTTAGAAGATCTGAGCTCTGAGTGGAAGGCGGTAAACCGTTTACTTCAAGAGCTGAGGGCAAAGCAGTGGCAG
AGAAAAATAGATGAGACCCTTGAAAGACTCCGGGAACTTCAAGAGGCCACGGATGAGCTGGACCTCAAGCTGCGC
CAAGCTGAGGTGATCAAGGGATCCTGGCAGCCCGTGGGCGATCTCCTCATTGACTCTCTCCAAGATCACCTCGAG
AAAGTCAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGAAGCTT
CTGCAGGTGGCCGTCGAGGACCGAGTCAGGCAGCTGCATGAAGCCCACAGGGACTTTGGTCCAGCATCTCAGCAC
TTTCTTTCCACGTCTGTCCAGGGTCCCTGGGAGAGAGCCATCTCGCCAAACAAAGTGCCCTACTATATCAACCAC
GAGACTCAAACAACTTGCTGGGACCATCCCAAAATGACAGAGCTCTACCAGTCTTTAGCTGACCTGAATAATGTC
AGATTCTCAGCTTATAGGACTGCCATGAAACTCCGAAGACTGCAGAAGGCCCTTTGCTTGGATCTCTTGAGCCTG
TCAGCTGCATGTGATGCCTTGGACCAGCACAACCTCAAGCAAAATGACCAGCCCATGGATATCCTGCAGATTATT
AATTGTTTGACCACTATTTATGACCGCCTGGAGCAAGAGCACAACAATTTGGTCAACGTCCCTCTCTGCGTGGAT
ATGTGTCTGAACTGGCTGCTGAATGTTTATGATACGGGACGAACAGGGAGGATCCGTGTCCTGTCTTTTAAAACT
GGCATCATTTCCCTGTGTAAAGCACATTTGGAAGACAAGTACAGATACCTTTTCAAGCAAGTGGCAAGTTCAACA
GGATTTTGTGACCAGCGCAGGCTGGGCCTCCTTCTGCATGATTCTATCCAAATTCCAAGACAGTTGGGTGAAGTT
GCATCCTTTGGGGGCAGTAACATTGAGCCAAGTGTCCGGAGCTGCTTCCAATTTGCTAATAATAAGCCAGAGATC
GAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTAC
AGGAGTCTAAAGCACTTTAATTATGACATCTGCCAAAGCTGCTTTTTTTCTGGTCGAGTTGCAAAAGGCCATAAA
ATGCACTATCCCATGGTGGAATATTGCACTCCGACTACATCAGGAGAAGATGTTCGAGACTTTGCCAAGGTACTA
AAAAACAAATTTCGAACCAAAAGGTATTTTGCGAAGCATCCCCGAATGGGCTACCTGCCAGTGCAGACTGTCTTA
GAGGGGGACAACATGGAAACTCCC
Human Sequences with codon optimization:
(SEQ ID NO: 86)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGCCGCTAGCGCCCGG
AGCGTGGAGAAGTGGCGGCGGTTCCACTACGACATCAAGATCTTCAACCAGTGGCTGACCGAGGCCGAGCAGTTC
CTGCGGAAGACCCAGATCCCCGAGAACTGGGAGCACGCCAAGTACAAGTGGTACCTGAAGGAGCTGCAGGACGGC
ATCGGCCAGCGGCAGACCGTGGTGCGGACCCTGAACGCCACCGGCGAGGAGATCATCCAGCAGAGCAGCAAGACC
GACGCCAGCATCCTGCAGGAGAAGCTGGGCAGCCTGAACCTGCGGTGGCAGGAGGTGTGCAAGCAGCTGAGCGAC
CGGAAGAAGCGGCTGGAGGAGCAGAAGAACATCCTGAGCGAGTTCCAGCGGGACCTGAACGAGTTCGTGCTGTGG
CTGGAGGAGGCCGACAACATCGCCAGCATCCCCCTGGAGCCCGGCAAGGAGCAGCAGCTGAAGGAGAAGCTGGAG
CAGGTGAAGCTGCTGGTGGAGGAGCTGCCCCTGCGGCAGGGCATCCTGAAGCAGCTGAACGAGACCGGCGGCCCC
GTGCTGGTGAGCGCCCCCATCAGCCCCGAGGAGCAGGACAAGCTGGAGAACAAGCTGAAGCAGACCAACCTGCAG
TGGATCAAGGTGAGCCGGGCCCTGCCCGAGAAGCAGGGCGAGATCGAGGCCCAGATCAAGGACCTGGGCCAGCTG
GAGAAGAAGCTGGAGGACCTGGAGGAGCAGCTGAACCACCTGCTGCTGTGGCTGAGCCCCATCCGGAACCAGCTG
GAGATCTACAACCAGCCCAACCAGGAGGGCCCCTTCGACGTGAAGGAGACCGAGATCGCCGTGCAGGCCAAGCAG
CCCGACGTGGAGGAGATCCTGAGCAAGGGCCAGCACCTGTACAAGGAGAAGCCCGCCACCCAGCCCGTGAAGCGG
AAGCTGGAGGACCTGAGCAGCGAGTGGAAGGCCGTGAACCGGCTGCTGCAGGAGCTGCGGGCCAAGCAGTGGCAG
AGAAAAATAGATGAGACCCTTGAAAGACTCCGGGAACTTCAAGAGGCCACGGATGAGCTGGACCTCAAGCTGCGC
CAAGCTGAGGTGATCAAGGGATCCTGGCAGCCCGTGGGCGATCTCCTCATTGACTCTCTCCAAGATCACCTCGAG
AAAGTCAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGAAGCTT
CTGCAGGTGGCCGTCGAGGACCGAGTCAGGCAGCTGCATGAAGCCCACAGGGACTTTGGTCCAGCATCTCAGCAC
TTTCTTTCCACGTCTGTCCAGGGTCCCTGGGAGAGAGCCATCTCGCCAAACAAAGTGCCCTACTATATCAACCAC
GAGACTCAAACAACTTGCTGGGACCATCCCAAAATGACAGAGCTCTACCAGTCTTTAGCTGACCTGAATAATGTC
AGATTCTCAGCTTATAGGACTGCCATGAAACTCCGAAGACTGCAGAAGGCCCTTTGCTTGGATCTCTTGAGCCTG
TCAGCTGCATGTGATGCCTTGGACCAGCACAACCTCAAGCAAAATGACCAGCCCATGGATATCCTGCAGATTATT
AATTGTTTGACCACTATTTATGACCGCCTGGAGCAAGAGCACAACAATTTGGTCAACGTCCCTCTCTGCGTGGAT
ATGTGTCTGAACTGGCTGCTGAATGTTTATGATACGGGACGAACAGGGAGGATCCGTGTCCTGTCTTTTAAAACT
GGCATCATTTCCCTGTGTAAAGCACATTTGGAAGACAAGTACAGATACCTTTTCAAGCAAGTGGCAAGTTCAACA
GGATTTTGTGACCAGCGCAGGCTGGGCCTCCTTCTGCATGATTCTATCCAAATTCCAAGACAGTTGGGTGAAGTT
GCATCCTTTGGGGGCAGTAACATTGAGCCAAGTGTCCGGAGCTGCTTCCAATTTGCTAATAATAAGCCAGAGATC
GAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTAC
AGGAGTCTAAAGCACTTTAATTATGACATCTGCCAAAGCTGCTTTTTTTCTGGTCGAGTTGCAAAAGGCCATAAA
ATGCACTATCCCATGGTGGAATATTGCACTCCGACTACATCAGGAGAAGATGTTCGAGACTTTGCCAAGGTACTA
AAAAACAAATTTCGAACCAAAAGGTATTTTGCGAAGCATCCCCGAATGGGCTACCTGCCAGTGCAGACTGTCTTA
GAGGGGGACAACATGGAAACTCCC
Utrophin Nterm-R1_Dystrophin R17-R19_H4 to 1st coiled coil and
proline rich region that follows:
Human Sequences without codon alterations:
(SEQ ID NO: 87)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGCCGCTAGCGCCAGA
TCTGTTGAGAAATGGCGGCGTTTTCATTATGATATAAAGATATTTAATCAGTGGCTAACAGAAGCTGAACAGTTT
CTCAGAAAGACACAAATTCCTGAGAATTGGGAACATGCTAAATACAAATGGTATCTTAAGGAACTCCAGGATGGC
ATTGGGCAGCGGCAAACTGTTGTCAGAACATTGAATGCAACTGGGGAAGAAATAATTCAGCAATCCTCAAAAACA
GATGCCAGTATTCTACAGGAAAAATTGGGAAGCCTGAATCTGCGGTGGCAGGAGGTCTGCAAACAGCTGTCAGAC
AGAAAAAAGAGGCTAGAAGAACAAAAGAATATCTTGTCAGAATTTCAAAGAGATTTAAATGAATTTGTTTTATGG
TTGGAGGAAGCAGATAACATTGCTAGTATCCCACTTGAACCTGGAAAAGAGCAGCAACTAAAAGAAAAGCTTGAG
CAAGTCAAGTTACTGGTGGAAGAGTTGCCCCTGCGCCAGGGAATTCTCAAACAATTAAATGAAACTGGAGGACCC
GTGCTTGTAAGTGCTCCCATAAGCCCAGAAGAGCAAGATAAACTTGAAAATAAGCTCAAGCAGACAAATCTCCAG
TGGATAAAGGTTTCCAGAGCTTTACCTGAGAAACAAGGAGAAATTGAAGCTCAAATAAAAGACCTTGGGCAGCTT
GAAAAAAAGCTTGAAGACCTTGAAGAGCAGTTAAATCATCTGCTGCTGTGGTTATCTCCTATTAGGAATCAGTTG
GAAATTTATAACCAACCAAACCAAGAAGGACCATTTGACGTTAAGGAAACTGAAATAGCAGTTCAAGCTAAACAA
CCGGATGTGGAAGAGATTTTGTCTAAAGGGCAGCATTTGTACAAGGAAAAACCAGCCACTCAGCCAGTGAAGAGG
AAGTTAGAAGATCTGAGCTCTGAGTGGAAGGCGGTAAACCGTTTACTTCAAGAGCTGAGGGCAAAGCAGGGTCCA
GCATCTCAGCACTTTCTTTCCACGTCTGTCCAGGGTCCCTGGGAGAGAGCCATCTCGCCAAACAAAGTGCCCTAC
TATATCAACCACGAGACTCAAACAACTTGCTGGGACCATCCCAAAATGACAGAGCTCTACCAGTCTTTAGCTGAC
CTGAATAATGTCAGATTCTCAGCTTATAGGACTGCCATGAAACTCCGAAGACTGCAGAAGGCCCTTTGCTTGGAT
CTCTTGAGCCTGTCAGCTGCATGTGATGCCTTGGACCAGCACAACCTCAAGCAAAATGACCAGCCCATGGATATC
CTGCAGATTATTAATTGTTTGACCACTATTTATGACCGCCTGGAGCAAGAGCACAACAATTTGGTCAACGTCCCT
CTCTGCGTGGATATGTGTCTGAACTGGCTGCTGAATGTTTATGATACGGGACGAACAGGGAGGATCCGTGTCCTG
TCTTTTAAAACTGGCATCATTTCCCTGTGTAAAGCACATTTGGAAGACAAGTACAGATACCTTTTCAAGCAAGTG
GCAAGTTCAACAGGATTTTGTGACCAGCGCAGGCTGGGCCTCCTTCTGCATGATTCTATCCAAATTCCAAGACAG
TTGGGTGAAGTTGCATCCTTTGGGGGCAGTAACATTGAGCCAAGTGTCCGGAGCTGCTTCCAATTTGCTAATAAT
AAGCCAGAGATCGAAGCGGCCCTCTTCCTAGACTGGATGAGACTGGAACCCCAGTCCATGGTGTGGCTGCCCGTC
CTGCACAGAGTGGCTGCTGCAGAAACTGCCAAGCATCAGGCCAAATGTAACATCTGCAAAGAGTGTCCAATCATT
GGATTCAGGTACAGGAGTCTAAAGCACTTTAATTATGACATCTGCCAAAGCTGCTTTTTTTCTGGTCGAGTTGCA
AAAGGCCATAAAATGCACTATCCCATGGTGGAATATTGCACTCCGACTACATCAGGAGAAGATGTTCGAGACTTT
GCCAAGGTACTAAAAAACAAATTTCGAACCAAAAGGTATTTTGCGAAGCATCCCCGAATGGGCTACCTGCCAGTG
CAGACTGTCTTAGAGGGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGCAGGCTAGCAGAAATG
GAAAACAGCAATGGATCTTATCTAAATGATAGCATCTCTCCTAATGAGAGCATAGATGATGAACATTTGTTAATC
CAGCATTACTGCCAAAGTTTGAACCAGGACTCCCCCCTGAGCCAGCCTCGTAGTCCTGCCCAGATCTTGATTTCC
TTAGAGAGTGAGGAAAGAGGGGAGCTAGAGAGAATCCTAGCAGATCTTGAGGAAGAAAACAGGAATCTGCAAGCA
GAATATGACCGTCTAAAGCAGCAGCACGAACATAAAGGCCTGTCCCCACTGCCGTCCCCTCCTGAAATGATGCCC
ACCTCTCCCCAGAGTCCC
Human Sequences with codon optimization:
(SEQ ID NO: 88)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGCCGCTAGCGCCCGG
AGCGTGGAGAAGTGGCGGCGGTTCCACTACGACATCAAGATCTTCAACCAGTGGCTGACCGAGGCCGAGCAGTTC
CTGCGGAAGACCCAGATCCCCGAGAACTGGGAGCACGCCAAGTACAAGTGGTACCTGAAGGAGCTGCAGGACGGC
ATCGGCCAGCGGCAGACCGTGGTGCGGACCCTGAACGCCACCGGCGAGGAGATCATCCAGCAGAGCAGCAAGACC
GACGCCAGCATCCTGCAGGAGAAGCTGGGCAGCCTGAACCTGCGGTGGCAGGAGGTGTGCAAGCAGCTGAGCGAC
CGGAAGAAGCGGCTGGAGGAGCAGAAGAACATCCTGAGCGAGTTCCAGCGGGACCTGAACGAGTTCGTGCTGTGG
CTGGAGGAGGCCGACAACATCGCCAGCATCCCCCTGGAGCCCGGCAAGGAGCAGCAGCTGAAGGAGAAGCTGGAG
CAGGTGAAGCTGCTGGTGGAGGAGCTGCCCCTGCGGCAGGGCATCCTGAAGCAGCTGAACGAGACCGGCGGCCCC
GTGCTGGTGAGCGCCCCCATCAGCCCCGAGGAGCAGGACAAGCTGGAGAACAAGCTGAAGCAGACCAACCTGCAG
TGGATCAAGGTGAGCCGGGCCCTGCCCGAGAAGCAGGGCGAGATCGAGGCCCAGATCAAGGACCTGGGCCAGCTG
GAGAAGAAGCTGGAGGACCTGGAGGAGCAGCTGAACCACCTGCTGCTGTGGCTGAGCCCCATCCGGAACCAGCTG
GAGATCTACAACCAGCCCAACCAGGAGGGCCCCTTCGACGTGAAGGAGACCGAGATCGCCGTGCAGGCCAAGCAG
CCCGACGTGGAGGAGATCCTGAGCAAGGGCCAGCACCTGTACAAGGAGAAGCCCGCCACCCAGCCCGTGAAGCGG
AAGCTGGAGGACCTGAGCAGCGAGTGGAAGGCCGTGAACCGGCTGCTGCAGGAGCTGCGGGCCAAGCAGGGTCCA
GCATCTCAGCACTTTCTTTCCACGTCTGTCCAGGGTCCCTGGGAGAGAGCCATCTCGCCAAACAAAGTGCCCTAC
TATATCAACCACGAGACTCAAACAACTTGCTGGGACCATCCCAAAATGACAGAGCTCTACCAGTCTTTAGCTGAC
CTGAATAATGTCAGATTCTCAGCTTATAGGACTGCCATGAAACTCCGAAGACTGCAGAAGGCCCTTTGCTTGGAT
CTCTTGAGCCTGTCAGCTGCATGTGATGCCTTGGACCAGCACAACCTCAAGCAAAATGACCAGCCCATGGATATC
CTGCAGATTATTAATTGTTTGACCACTATTTATGACCGCCTGGAGCAAGAGCACAACAATTTGGTCAACGTCCCT
CTCTGCGTGGATATGTGTCTGAACTGGCTGCTGAATGTTTATGATACGGGACGAACAGGGAGGATCCGTGTCCTG
TCTTTTAAAACTGGCATCATTTCCCTGTGTAAAGCACATTTGGAAGACAAGTACAGATACCTTTTCAAGCAAGTG
GCAAGTTCAACAGGATTTTGTGACCAGCGCAGGCTGGGCCTCCTTCTGCATGATTCTATCCAAATTCCAAGACAG
TTGGGTGAAGTTGCATCCTTTGGGGGCAGTAACATTGAGCCAAGTGTCCGGAGCTGCTTCCAATTTGCTAATAAT
AAGCCAGAGATCGAAGCGGCCCTCTTCCTAGACTGGATGAGACTGGAACCCCAGTCCATGGTGTGGCTGCCCGTC
CTGCACAGAGTGGCTGCTGCAGAAACTGCCAAGCATCAGGCCAAATGTAACATCTGCAAAGAGTGTCCAATCATT
GGATTCAGGTACAGGAGTCTAAAGCACTTTAATTATGACATCTGCCAAAGCTGCTTTTTTTCTGGTCGAGTTGCA
AAAGGCCATAAAATGCACTATCCCATGGTGGAATATTGCACTCCGACTACATCAGGAGAAGATGTTCGAGACTTT
GCCAAGGTACTAAAAAACAAATTTCGAACCAAAAGGTATTTTGCGAAGCATCCCCGAATGGGCTACCTGCCAGTG
CAGACTGTCTTAGAGGGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGCAGGCTAGCAGAAATG
GAAAACAGCAATGGATCTTATCTAAATGATAGCATCTCTCCTAATGAGAGCATAGATGATGAACATTTGTTAATC
CAGCATTACTGCCAAAGTTTGAACCAGGACTCCCCCCTGAGCCAGCCTCGTAGTCCTGCCCAGATCTTGATTTCC
TTAGAGAGTGAGGAAAGAGGGGAGCTAGAGAGAATCCTAGCAGATCTTGAGGAAGAAAACAGGAATCTGCAAGCA
GAATATGACCGTCTAAAGCAGCAGCACGAACATAAAGGCCTGTCCCCACTGCCGTCCCCTCCTGAAATGATGCCC
ACCTCTCCCCAGAGTCCC
Utrophin N-terminus through R1_Dystrophin R17-R19_H4 to end of
syntrophin region:
Human Sequences without codon alterations:
(SEQ ID NO: 89)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGCCGCTAGCGCCAGA
TCTGTTGAGAAATGGCGGCGTTTTCATTATGATATAAAGATATTTAATCAGTGGCTAACAGAAGCTGAACAGTTT
CTCAGAAAGACACAAATTCCTGAGAATTGGGAACATGCTAAATACAAATGGTATCTTAAGGAACTCCAGGATGGC
ATTGGGCAGCGGCAAACTGTTGTCAGAACATTGAATGCAACTGGGGAAGAAATAATTCAGCAATCCTCAAAAACA
GATGCCAGTATTCTACAGGAAAAATTGGGAAGCCTGAATCTGCGGTGGCAGGAGGTCTGCAAACAGCTGTCAGAC
AGAAAAAAGAGGCTAGAAGAACAAAAGAATATCTTGTCAGAATTTCAAAGAGATTTAAATGAATTTGTTTTATGG
TTGGAGGAAGCAGATAACATTGCTAGTATCCCACTTGAACCTGGAAAAGAGCAGCAACTAAAAGAAAAGCTTGAG
CAAGTCAAGTTACTGGTGGAAGAGTTGCCCCTGCGCCAGGGAATTCTCAAACAATTAAATGAAACTGGAGGACCC
GTGCTTGTAAGTGCTCCCATAAGCCCAGAAGAGCAAGATAAACTTGAAAATAAGCTCAAGCAGACAAATCTCCAG
TGGATAAAGGTTTCCAGAGCTTTACCTGAGAAACAAGGAGAAATTGAAGCTCAAATAAAAGACCTTGGGCAGCTT
GAAAAAAAGCTTGAAGACCTTGAAGAGCAGTTAAATCATCTGCTGCTGTGGTTATCTCCTATTAGGAATCAGTTG
GAAATTTATAACCAACCAAACCAAGAAGGACCATTTGACGTTAAGGAAACTGAAATAGCAGTTCAAGCTAAACAA
CCGGATGTGGAAGAGATTTTGTCTAAAGGGCAGCATTTGTACAAGGAAAAACCAGCCACTCAGCCAGTGAAGAGG
AAGTTAGAAGATCTGAGCTCTGAGTGGAAGGCGGTAAACCGTTTACTTCAAGAGCTGAGGGCAAAGCAGGGTCCA
GCATCTCAGCACTTTCTTTCCACGTCTGTCCAGGGTCCCTGGGAGAGAGCCATCTCGCCAAACAAAGTGCCCTAC
TATATCAACCACGAGACTCAAACAACTTGCTGGGACCATCCCAAAATGACAGAGCTCTACCAGTCTTTAGCTGAC
CTGAATAATGTCAGATTCTCAGCTTATAGGACTGCCATGAAACTCCGAAGACTGCAGAAGGCCCTTTGCTTGGAT
CTCTTGAGCCTGTCAGCTGCATGTGATGCCTTGGACCAGCACAACCTCAAGCAAAATGACCAGCCCATGGATATC
CTGCAGATTATTAATTGTTTGACCACTATTTATGACCGCCTGGAGCAAGAGCACAACAATTTGGTCAACGTCCCT
CTCTGCGTGGATATGTGTCTGAACTGGCTGCTGAATGTTTATGATACGGGACGAACAGGGAGGATCCGTGTCCTG
TCTTTTAAAACTGGCATCATTTCCCTGTGTAAAGCACATTTGGAAGACAAGTACAGATACCTTTTCAAGCAAGTG
GCAAGTTCAACAGGATTTTGTGACCAGCGCAGGCTGGGCCTCCTTCTGCATGATTCTATCCAAATTCCAAGACAG
TTGGGTGAAGTTGCATCCTTTGGGGGCAGTAACATTGAGCCAAGTGTCCGGAGCTGCTTCCAATTTGCTAATAAT
AAGCCAGAGATCGAAGCGGCCCTCTTCCTAGACTGGATGAGACTGGAACCCCAGTCCATGGTGTGGCTGCCCGTC
CTGCACAGAGTGGCTGCTGCAGAAACTGCCAAGCATCAGGCCAAATGTAACATCTGCAAAGAGTGTCCAATCATT
GGATTCAGGTACAGGAGTCTAAAGCACTTTAATTATGACATCTGCCAAAGCTGCTTTTTTTCTGGTCGAGTTGCA
AAAGGCCATAAAATGCACTATCCCATGGTGGAATATTGCACTCCGACTACATCAGGAGAAGATGTTCGAGACTTT
GCCAAGGTACTAAAAAACAAATTTCGAACCAAAAGGTATTTTGCGAAGCATCCCCGAATGGGCTACCTGCCAGTG
CAGACTGTCTTAGAGGGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGCAGGCTAGCAGAAATG
GAAAACAGCAATGGATCTTATCTAAATGATAGCATCTCTCCTAATGAGAGCATAGATGATGAACATTTGTTAATC
CAGCATTACTGCCAAAGTTTGAACCAGGACTCCCCCCTGAGCCAGCCTCGTAGT
Human Sequences without codon alterations:
(SEQ ID NO: 90)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGCCGCTAGCGCCCGG
AGCGTGGAGAAGTGGCGGCGGTTCCACTACGACATCAAGATCTTCAACCAGTGGCTGACCGAGGCCGAGCAGTTC
CTGCGGAAGACCCAGATCCCCGAGAACTGGGAGCACGCCAAGTACAAGTGGTACCTGAAGGAGCTGCAGGACGGC
ATCGGCCAGCGGCAGACCGTGGTGCGGACCCTGAACGCCACCGGCGAGGAGATCATCCAGCAGAGCAGCAAGACC
GACGCCAGCATCCTGCAGGAGAAGCTGGGCAGCCTGAACCTGCGGTGGCAGGAGGTGTGCAAGCAGCTGAGCGAC
CGGAAGAAGCGGCTGGAGGAGCAGAAGAACATCCTGAGCGAGTTCCAGCGGGACCTGAACGAGTTCGTGCTGTGG
CTGGAGGAGGCCGACAACATCGCCAGCATCCCCCTGGAGCCCGGCAAGGAGCAGCAGCTGAAGGAGAAGCTGGAG
CAGGTGAAGCTGCTGGTGGAGGAGCTGCCCCTGCGGCAGGGCATCCTGAAGCAGCTGAACGAGACCGGCGGCCCC
GTGCTGGTGAGCGCCCCCATCAGCCCCGAGGAGCAGGACAAGCTGGAGAACAAGCTGAAGCAGACCAACCTGCAG
TGGATCAAGGTGAGCCGGGCCCTGCCCGAGAAGCAGGGCGAGATCGAGGCCCAGATCAAGGACCTGGGCCAGCTG
GAGAAGAAGCTGGAGGACCTGGAGGAGCAGCTGAACCACCTGCTGCTGTGGCTGAGCCCCATCCGGAACCAGCTG
GAGATCTACAACCAGCCCAACCAGGAGGGCCCCTTCGACGTGAAGGAGACCGAGATCGCCGTGCAGGCCAAGCAG
CCCGACGTGGAGGAGATCCTGAGCAAGGGCCAGCACCTGTACAAGGAGAAGCCCGCCACCCAGCCCGTGAAGCGG
AAGCTGGAGGACCTGAGCAGCGAGTGGAAGGCCGTGAACCGGCTGCTGCAGGAGCTGCGGGCCAAGCAGGGTCCA
GCATCTCAGCACTTTCTTTCCACGTCTGTCCAGGGTCCCTGGGAGAGAGCCATCTCGCCAAACAAAGTGCCCTAC
TATATCAACCACGAGACTCAAACAACTTGCTGGGACCATCCCAAAATGACAGAGCTCTACCAGTCTTTAGCTGAC
CTGAATAATGTCAGATTCTCAGCTTATAGGACTGCCATGAAACTCCGAAGACTGCAGAAGGCCCTTTGCTTGGAT
CTCTTGAGCCTGTCAGCTGCATGTGATGCCTTGGACCAGCACAACCTCAAGCAAAATGACCAGCCCATGGATATC
CTGCAGATTATTAATTGTTTGACCACTATTTATGACCGCCTGGAGCAAGAGCACAACAATTTGGTCAACGTCCCT
CTCTGCGTGGATATGTGTCTGAACTGGCTGCTGAATGTTTATGATACGGGACGAACAGGGAGGATCCGTGTCCTG
TCTTTTAAAACTGGCATCATTTCCCTGTGTAAAGCACATTTGGAAGACAAGTACAGATACCTTTTCAAGCAAGTG
GCAAGTTCAACAGGATTTTGTGACCAGCGCAGGCTGGGCCTCCTTCTGCATGATTCTATCCAAATTCCAAGACAG
TTGGGTGAAGTTGCATCCTTTGGGGGCAGTAACATTGAGCCAAGTGTCCGGAGCTGCTTCCAATTTGCTAATAAT
AAGCCAGAGATCGAAGCGGCCCTCTTCCTAGACTGGATGAGACTGGAACCCCAGTCCATGGTGTGGCTGCCCGTC
CTGCACAGAGTGGCTGCTGCAGAAACTGCCAAGCATCAGGCCAAATGTAACATCTGCAAAGAGTGTCCAATCATT
GGATTCAGGTACAGGAGTCTAAAGCACTTTAATTATGACATCTGCCAAAGCTGCTTTTTTTCTGGTCGAGTTGCA
AAAGGCCATAAAATGCACTATCCCATGGTGGAATATTGCACTCCGACTACATCAGGAGAAGATGTTCGAGACTTT
GCCAAGGTACTAAAAAACAAATTTCGAACCAAAAGGTATTTTGCGAAGCATCCCCGAATGGGCTACCTGCCAGTG
CAGACTGTCTTAGAGGGGGACAACATGGAAACTCCCGTTACTCTGATCAACTTCTGGCCAGTAGATTCTGCGCCT
GCCTCGTCCCCTCAGCTTTCACACGATGATACTCATTCACGCATTGAACATTATGCTAGCAGGCTAGCAGAAATG
GAAAACAGCAATGGATCTTATCTAAATGATAGCATCTCTCCTAATGAGAGCATAGATGATGAACATTTGTTAATC
CAGCATTACTGCCAAAGTTTGAACCAGGACTCCCCCCTGAGCCAGCCTCGTAGT
Utrophin N-terminus through R1_Dystrophin R23 to end of Dystrophin:
Human Sequences without codon alterations:
(SEQ ID NO: 91)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGCCGCTAGCGCCAGT
TCTGACCAGTGGAAGCGTCTGCACCTTTCTCTGCAGGAACTTCTGGTGTGGCTACAGCTGAAAGATGATGAATTA
AGCCGGCAGGCACCTATTGGAGGCGACTTTCCAGCAGTTCAGAAGCAGAACGATGTACATAGGGCCTTCAAGAGG
GAATTGAAAACTAAAGAACCTGTAATCATGAGTACTCTTGAGACTGTACGAATATTTCTGACAGAGCAGCCTTTG
GAAGGACTAGAGAAACTCTACCAGGAGCCCAGAGAGCTGCCTCCTGAGGAGAGAGCCCAGAATGTCACTCGGCTT
CTACGAAAGCAGGCTGAGGAGGTCAATACTGAGTGGGAAAAATTGAACCTGCACTCCGCTGACTGGCAGAGAAAA
ATAGATGAGACCCTTGAAAGACTCCGGGAACTTCAAGAGGCCACGGATGAGCTGGACCTCAAGCTGCGCCAAGCT
GAGGTGATCAAGGGATCCTGGCAGCCCGTGGGCGATCTCCTCATTGACTCTCTCCAAGATCACCTCGAGAAAGTC
AAGGCACTTCGAGGAGAAATTGCGCCTCTGAAAGAGAACGTGAGCCACGTCAATGACCTTGCTCGCCAGCTTACC
ACTTTGGGCATTCAGCTCTCACCGTATAACCTCAGCACTCTGGAAGACCTGAACACCAGATGGAAGCTTCTGCAG
GTGGCCGTCGAGGACCGAGTCAGGCAGCTGCATGAAGCCCACAGGGACTTTGGTCCAGCATCTCAGCACTTTCTT
TCCACGTCTGTCCAGGGTCCCTGGGAGAGAGCCATCTCGCCAAACAAAGTGCCCTACTATATCAACCACGAGACT
CAAACAACTTGCTGGGACCATCCCAAAATGACAGAGCTCTACCAGTCTTTAGCTGACCTGAATAATGTCAGATTC
TCAGCTTATAGGACTGCCATGAAACTCCGAAGACTGCAGAAGGCCCTTTGCTTGGATCTCTTGAGCCTGTCAGCT
GCATGTGATGCCTTGGACCAGCACAACCTCAAGCAAAATGACCAGCCCATGGATATCCTGCAGATTATTAATTGT
TTGACCACTATTTATGACCGCCTGGAGCAAGAGCACAACAATTTGGTCAACGTCCCTCTCTGCGTGGATATGTGT
CTGAACTGGCTGCTGAATGTTTATGATACGGGACGAACAGGGAGGATCCGTGTCCTGTCTTTTAAAACTGGCATC
ATTTCCCTGTGTAAAGCACATTTGGAAGACAAGTACAGATACCTTTTCAAGCAAGTGGCAAGTTCAACAGGATTT
TGTGACCAGCGCAGGCTGGGCCTCCTTCTGCATGATTCTATCCAAATTCCAAGACAGTTGGGTGAAGTTGCATCC
TTTGGGGGCAGTAACATTGAGCCAAGTGTCCGGAGCTGCTTCCAATTTGCTAATAATAAGCCAGAGATCGAAGCG
GCCCTCTTCCTAGACTGGATGAGACTGGAACCCCAGTCCATGGTGTGGCTGCCCGTCCTGCACAGAGTGGCTGCT
GCAGAAACTGCCAAGCATCAGGCCAAATGTAACATCTGCAAAGAGTGTCCAATCATTGGATTCAGGTACAGGAGT
CTAAAGCACTTTAATTATGACATCTGCCAAAGCTGCTTTTTTTCTGGTCGAGTTGCAAAAGGCCATAAAATGCAC
TATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGG
GACAACATGGAAACTCCCGTTACTCTGATCAACTTCTGGCCAGTAGATTCTGCGCCTGCCTCGTCCCCTCAGCTT
TCACACGATGATACTCATTCACGCATTGAACATTATGCTAGCAGGCTAGCAGAAATGGAAAACAGCAATGGATCT
TATCTAAATGATAGCATCTCTCCTAATGAGAGCATAGATGATGAACATTTGTTAATCCAGCATTACTGCCAAAGT
TTGAACCAGGACTCCCCCCTGAGCCAGCCTCGTAGTCCTGCCCAGATCTTGATTTCCTTAGAGAGTGAGGAAAGA
GGGGAGCTAGAGAGAATCCTAGCAGATCTTGAGGAAGAAAACAGGAATCTGCAAGCAGAATATGACCGTCTAAAG
CAGCAGCACGAACATAAAGGCCTGTCCCCACTGCCGTCCCCTCCTGAAATGATGCCCACCTCTCCCCAGAGTCCC
CGGGATGCTGAGCTCATTGCTGAGGCCAAGCTACTGCGTCAACACAAAGGCCGCCTGGAAGCCAGGATGCAAATC
CTGGAAGACCACAATAAACAGCTGGAGTCACAGTTACACAGGCTAAGGCAGCTGCTGGAGCAACCCCAGGCAGAG
GCCAAAGTGAATGGCACAACGGTGTCCTCTCCTTCTACCTCTCTACAGAGGTCCGACAGCAGTCAGCCTATGCTG
CTCCGAGTGGTTGGCAGTCAAACTTCGGACTCCATGGGTGAGGAAGATCTTCTCAGTCCTCCCCAGGACACAAGC
ACAGGGTTAGAGGAGGTGATGGAGCAACTCAACAACTCCTTCCCTAGTTCAAGAGGAAGAAATACCCCTGGAAAG
CCAATGAGAGAGGACACAATG
Human Sequences without codon alterations:
(SEQ ID NO: 92)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGCCGCTAGCGCCAGC
AGCGACCAGTGGAAGCGGCTGCACCTGAGCCTGCAGGAGCTGCTGGTGTGGCTGCAGCTGAAGGACGACGAGCTG
AGCCGGCAGGCCCCCATCGGCGGCGACTTCCCCGCCGTGCAGAAGCAGAACGACGTGCACCGGGCCTTCAAGCGG
GAGCTGAAGACCAAGGAGCCCGTGATCATGAGCACCCTGGAGACCGTGCGGATCTTCCTGACCGAGCAGCCCCTG
GAGGGCCTGGAGAAGCTGTACCAGGAGCCCCGGGAGCTGCCCCCCGAGGAGCGGGCCCAGAACGTGACCCGGCTG
CTGCGGAAGCAGGCCGAGGAGGTGAACACCGAGTGGGAGAAGCTGAACCTGCACAGCGCCGACTGGCAGAGAAAA
ATAGATGAGACCCTTGAAAGACTCCGGGAACTTCAAGAGGCCACGGATGAGCTGGACCTCAAGCTGCGCCAAGCT
GAGGTGATCAAGGGATCCTGGCAGCCCGTGGGCGATCTCCTCATTGACTCTCTCCAAGATCACCTCGAGAAAGTC
AAGGCACTTCGAGGAGAAATTGCGCCTCTGAAAGAGAACGTGAGCCACGTCAATGACCTTGCTCGCCAGCTTACC
ACTTTGGGCATTCAGCTCTCACCGTATAACCTCAGCACTCTGGAAGACCTGAACACCAGATGGAAGCTTCTGCAG
GTGGCCGTCGAGGACCGAGTCAGGCAGCTGCATGAAGCCCACAGGGACTTTGGTCCAGCATCTCAGCACTTTCTT
TCCACGTCTGTCCAGGGTCCCTGGGAGAGAGCCATCTCGCCAAACAAAGTGCCCTACTATATCAACCACGAGACT
CAAACAACTTGCTGGGACCATCCCAAAATGACAGAGCTCTACCAGTCTTTAGCTGACCTGAATAATGTCAGATTC
TCAGCTTATAGGACTGCCATGAAACTCCGAAGACTGCAGAAGGCCCTTTGCTTGGATCTCTTGAGCCTGTCAGCT
GCATGTGATGCCTTGGACCAGCACAACCTCAAGCAAAATGACCAGCCCATGGATATCCTGCAGATTATTAATTGT
TTGACCACTATTTATGACCGCCTGGAGCAAGAGCACAACAATTTGGTCAACGTCCCTCTCTGCGTGGATATGTGT
CTGAACTGGCTGCTGAATGTTTATGATACGGGACGAACAGGGAGGATCCGTGTCCTGTCTTTTAAAACTGGCATC
ATTTCCCTGTGTAAAGCACATTTGGAAGACAAGTACAGATACCTTTTCAAGCAAGTGGCAAGTTCAACAGGATTT
TGTGACCAGCGCAGGCTGGGCCTCCTTCTGCATGATTCTATCCAAATTCCAAGACAGTTGGGTGAAGTTGCATCC
TTTGGGGGCAGTAACATTGAGCCAAGTGTCCGGAGCTGCTTCCAATTTGCTAATAATAAGCCAGAGATCGAAGCG
GCCCTCTTCCTAGACTGGATGAGACTGGAACCCCAGTCCATGGTGTGGCTGCCCGTCCTGCACAGAGTGGCTGCT
GCAGAAACTGCCAAGCATCAGGCCAAATGTAACATCTGCAAAGAGTGTCCAATCATTGGATTCAGGTACAGGAGT
CTAAAGCACTTTAATTATGACATCTGCCAAAGCTGCTTTTTTTCTGGTCGAGTTGCAAAAGGCCATAAAATGCAC
TATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGG
GACAACATGGAAACTCCCGTTACTCTGATCAACTTCTGGCCAGTAGATTCTGCGCCTGCCTCGTCCCCTCAGCTT
TCACACGATGATACTCATTCACGCATTGAACATTATGCTAGCAGGCTAGCAGAAATGGAAAACAGCAATGGATCT
TATCTAAATGATAGCATCTCTCCTAATGAGAGCATAGATGATGAACATTTGTTAATCCAGCATTACTGCCAAAGT
TTGAACCAGGACTCCCCCCTGAGCCAGCCTCGTAGTCCTGCCCAGATCTTGATTTCCTTAGAGAGTGAGGAAAGA
GGGGAGCTAGAGAGAATCCTAGCAGATCTTGAGGAAGAAAACAGGAATCTGCAAGCAGAATATGACCGTCTAAAG
CAGCAGCACGAACATAAAGGCCTGTCCCCACTGCCGTCCCCTCCTGAAATGATGCCCACCTCTCCCCAGAGTCCC
CGGGATGCTGAGCTCATTGCTGAGGCCAAGCTACTGCGTCAACACAAAGGCCGCCTGGAAGCCAGGATGCAAATC
CTGGAAGACCACAATAAACAGCTGGAGTCACAGTTACACAGGCTAAGGCAGCTGCTGGAGCAACCCCAGGCAGAG
GCCAAAGTGAATGGCACAACGGTGTCCTCTCCTTCTACCTCTCTACAGAGGTCCGACAGCAGTCAGCCTATGCTG
CTCCGAGTGGTTGGCAGTCAAACTTCGGACTCCATGGGTGAGGAAGATCTTCTCAGTCCTCCCCAGGACACAAGC
ACAGGGTTAGAGGAGGTGATGGAGCAACTCAACAACTCCTTCCCTAGTTCAAGAGGAAGAAATACCCCTGGAAAG
CCAATGAGAGAGGACACAATG

### Amino Acid Sequences for Exemplary Chimeric Proteins

In some embodiments, a micro-dystrophin protein as described herein further comprises one or more utrophin protein domains. In some embodiments, a micro-dystrophin protein optimized for skeletal muscle comprises a utrophin N-terminus. In some embodiments, a micro-dystrophin protein comprises the region of utrophin from the N-terminus to spectrin-like repeat 1. In some embodiments, a micro-dystrophin protein comprises the region of utrophin from the N-terminus to spectrin-like repeat 2. In some embodiments, a micro-dystrophin protein comprises the region of utrophin from the N-terminus to spectrin-like repeat 3. In some embodiments, the micro-dystrophin protein comprises the region of utrophin from the N-terminus to hinge 2.

Non-limiting examples of human chimeric utrophin/dystrophin micro-dystrophin proteins optimized for skeletal muscle are described below.

Utrophin N-terminus through-R2_Dystrophin R16-R17_R24+H4+trCterm:
(SEQ ID NO: 93)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EIAASAMMVMTEDMPLEISYVPSTYLTEITHVSQALLEVEQLLNAPDLCAKDFEDLFKQEESLKNIKDSLQQSSG
RIDIIHSKKTAALQSATPVERVKLQEALSQLDFQWEKVNKMYKDRQGRFDRSVEKWRRFHYDIKIFNQWLTEAEQ
FLRKTQIPENWEHAKYKWYLKELQDGIGQRQTVVRTLNATGEEIIQQSSKTDASILQEKLGSLNLRWQEVCKQLS
DRKKRLEEQKWQRKIDETLERLQELQEATDELDLKLRQAEVIKGSWQPVGDLLIDSLQDHLEKVKALRGEIAPLK
ENVSHVNDLARQLTTLGIQLSPYNLSTLEDLNTRWKLLQVAVEDRVRQLHEAHRDFGPASQHELSTSVQGPWERA
ISPNKVPYYINHETQTTCWDHPKMTELYQSLADLNNVRFSAYRTAMKLRRLQKALCLDLLSLSAACDALDQHNLK
QNDQPMDILQIINCLTTIYDRLEQEHNNLVNVPLCVDMCLNWLLNVYDTGRTGRIRVLSFKTGIISLCKAHLEDK
YRYLFKQVASSTGFCDQRRLGLLLHDSIQIPRQLGEVASFGGSNIEPSVRSCFQFANNKPEIEAALFLDWMRLEP
QSMVWLPVLHRVAAAETAKHQAKCNICKECPIIGFRYRSLKHENYDICQSCFFSGRVAKGHKMHYPMVEYCTPTT
SGEDVRDFAKVLKNKFRTKRYFAKHPRMGYLPVQTVLEGDNMETP
Utrophin N-terminus through R2_Dystrophin R16-R17_H4 to end of
syntrophin region:
(SEQ ID NO: 94)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EIAASAMMVMTEDMPLEISYVPSTYLTEITHVSQALLEVEQLLNAPDLCAKDFEDLFKQEESLKNIKDSLQQSSG
RIDIIHSKKTAALQSATPVERVKLQEALSQLDFQWEKVNKMYKDRQGRFDRSVEKWRRFHYDIKIFNQWLTEAEQ
FLRKTQIPENWEHAKYKWYLKELQDGIGQRQTVVRTLNATGEEIIQQSSKTDASILQEKLGSLNLRWQEVCKQLS
DRKKRLEEQKGPASQHELSTSVQGPWERAISPNKVPYYINHETQTTCWDHPKMTELYQSLADLNNVRESAYRTAM
KLRRLQKALCLDLLSLSAACDALDQHNLKQNDQPMDILQIINCLTTIYDRLEQEHNNLVNVPLCVDMCLNWLLNV
YDTGRTGRIRVLSFKTGIISLCKAHLEDKYRYLFKQVASSTGFCDQRRLGLLLHDSIQIPRQLGEVASFGGSNIE
PSVRSCFQFANNKPEIEAALFLDWMRLEPQSMVWLPVLHRVAAAETAKHQAKCNICKECPIIGFRYRSLKHENYD
ICQSCFFSGRVAKGHKMHYPMVEYCTPTTSGEDVRDFAKVLKNKFRTKRYFAKHPRMGYLPVQTVLEGDNMETPV
TLINFWPVDSAPASSPQLSHDDTHSRIEHYASRLAEMENSNGSYLNDSISPNESIDDEHLLIQHYCQSLNQDSPL
SQPRS
Utrophin N-terminus through R2_Dystrophin R16-R17_H4 to 1st
coiled coil and proline rich region that follows:
(SEQ ID NO: 95)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EIAASAMMVMTEDMPLEISYVPSTYLTEITHVSQALLEVEQLLNAPDLCAKDFEDLFKQEESLKNIKDSLQQSSG
RIDIIHSKKTAALQSATPVERVKLQEALSQLDFQWEKVNKMYKDRQGRFDRSVEKWRRFHYDIKIFNQWLTEAEQ
FLRKTQIPENWEHAKYKWYLKELQDGIGQRQTVVRILNATGEEIIQQSSKTDASILQEKLGSLNLRWQEVCKQLS
DRKKRLEEQKGPASQHELSTSVQGPWERAISPNKVPYYINHETQTTCWDHPKMTELYQSLADLNNVRFSAYRTAM
KLRRLQKALCLDLLSLSAACDALDQHNLKQNDQPMDILQIINCLTTIYDRLEQEHNNLVNVPLCVDMCLNWLLNV
YDTGRTGRIRVLSFKTGIISLCKAHLEDKYRYLFKQVASSTGFCDQRRLGLLLHDSIQIPRQLGEVASFGGSNIE
PSVRSCFQFANNKPEIEAALFLDWMRLEPQSMVWLPVLHRVAAAETAKHQAKCNICKECPIIGFRYRSLKHENYD
ICQSCFFSGRVAKGHKMHYPMVEYCTPTTSGEDVRDFAKVLKNKFRTKRYFAKHPRMGYLPVQTVLEGDNMETPV
TLINFWPVDSAPASSPQLSHDDTHSRIEHYASRLAEMENSNGSYLNDSISPNESIDDEHLLIQHYCQSLNQDSPL
SQPRSPAQILISLESEERGELERILADLEEENRNLQAEYDRLKQQHEHKGLSPLPSPPEMMPTSPQSP
Utrophin N-terminus through R1_Dystrophin R16-R17_R23 to end of
syntrophin region:
(SEQ ID NO: 96)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMAASAMMVMTEDMPLEISYVPSTYLTEITHV
SQALLEVEQLLNAPDLCAKDFEDLFKQEESLKNIKDSLQQSSGRIDIIHSKKTAALQSATPVERVKLQEALSQLD
FQWEKVNKMYKDRQGRFDRSVEKWRRFHYDIKIFNQWLTEAEQFLRKTQIPENWEHAKYKWYLKELQDGIGQRQT
VVRTLNATGEEIIQQSSKTDASILQEKLGSLNLRWQEVCKQLSDRKKRLEEQKSSDQWKRLHLSLQELLVWLQLK
DDELSRQAPIGGDFPAVQKQNDVHRAFKRELKTKEPVIMSTLETVRIFLTEQPLEGLEKLYQEPRELPPEERAQN
VTRLLRKQAEEVNTEWEKLNLHSADWQRKIDETLERLQELQEATDELDLKLRQAEVIKGSWQPVGDLLIDSLQDH
LEKVKALRGEIAPLKENVSHVNDLARQLTTLGIQLSPYNLSTLEDLNTRWKLLQVAVEDRVRQLHEAHRDFGPAS
QHELSTSVQGPWERAISPNKVPYYINHETQTTCWDHPKMTELYQSLADLNNVRESAYRTAMKLRRLQKALCLDLL
SLSAACDALDQHNLKQNDQPMDILQIINCLTTIYDRLEQEHNNLVNVPLCVDMCLNWLLNVYDTGRIGRIRVLSF
KTGIISLCKAHLEDKYRYLFKQVASSTGFCDQRRLGLLLHDSIQIPRQLGEVASEGGSNIEPSVRSCFQFANNKP
EIEAALFLDWMRLEPQSMVWLPVLHRVAAAETAKHQAKCNICKECPIIGFRYRSLKHFNYDICQSCFFSGRVAKG
HKMHYPMVEYCTPTTSGEDVRDFAKVLKNKFRTKRYFAKHPRMGYLPVQTVLEGDNMETPVTLINFWPVDSAPAS
SPQLSHDDTHSRIEHYASRLAEMENSNGSYLNDSISPNESIDDEHLLIQHYCQSLNQDSPLSQPRS
Utrophin N-terminus through R2_Dystrophin R16-R17_R24+H4 to end of
syntrophin region:
(SEQ ID NO: 97)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EIAASAMMVMTEDMPLEISYVPSTYLTEITHVSQALLEVEQLLNAPDLCAKDFEDLFKQEESLKNIKDSLQQSSG
RIDIIHSKKTAALQSATPVERVKLQEALSQLDFQWEKVNKMYKDRQGRFDRSVEKWRRFHYDIKIFNQWLTEAEQ
FLRKTQIPENWEHAKYKWYLKELQDGIGQRQTVVRTLNATGEEIIQQSSKTDASILQEKLGSLNLRWQEVCKQLS
DRKKRLEEQKWQRKIDETLERLQELQEATDELDLKLRQAEVIKGSWQPVGDLLIDSLQDHLEKVKALRGEIAPLK
ENVSHVNDLARQLTTLGIQLSPYNLSTLEDLNTRWKLLQVAVEDRVRQLHEAHRDFGPASQHELSTSVQGPWERA
ISPNKVPYYINHETQTTCWDHPKMTELYQSLADLNNVRESAYRTAMKLRRLQKALCLDLLSLSAACDALDQHNLK
QNDQPMDILQIINCLTTIYDRLEQEHNNLVNVPLCVDMCLNWLLNVYDTGRIGRIRVLSFKTGIISLCKAHLEDK
YRYLFKQVASSTGFCDQRRLGLLLHDSIQIPRQLGEVASFGGSNIEPSVRSCFQFANNKPEIEAALFLDWMRLEP
QSMVWLPVLHRVAAAETAKHQAKCNICKECPIIGFRYRSLKHFNYDICQSCFFSGRVAKGHKMHYPMVEYCTPTT
SGEDVRDFAKVLKNKFRTKRYFAKHPRMGYLPVQTVLEGDNMETPVTLINFWPVDSAPASSPQLSHDDTHSRIEH
YASRLAEMENSNGSYLNDSISPNESIDDEHLLIQHYCQSLNQDSPLSQPRS
Utrophin N-terminus through R2_Dystrophin R23+R24+H4 to end of
syntrophin region:
(SEQ ID NO: 98)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EIAASALQLKDDELSRQAPIGGDFPAVQKQNDVHRAFKRELKTKEPVIMSTLETVRIFLTEQPLEGLEKLYQEPR
ELPPEERAQNVTRLLRKQAEEVNTEWEKLNLHSADWQRKIDETLERLQELQEATDELDLKLRQAEVIKGSWQPVG
DLLIDSLQDHLEKVKALRGEIAPLKENVSHVNDLARQLTTLGIQLSPYNLSTLEDLNTRWKLLQVAVEDRVRQLH
EAHRDFGPASQHELSTSVQGPWERAISPNKVPYYINHETQTTCWDHPKMTELYQSLADLNNVRESAYRTAMKLRR
LQKALCLDLLSLSAACDALDQHNLKQNDQPMDILQIINCLTTIYDRLEQEHNNLVNVPLCVDMCLNWLLNVYDTG
RTGRIRVLSFKTGIISLCKAHLEDKYRYLFKQVASSTGFCDQRRLGLLLHDSIQIPRQLGEVASEGGSNIEPSVR
SCFQFANNKPEIEAALFLDWMRLEPQSMVWLPVLHRVAAAETAKHQAKCNICKECPIIGFRYRSLKHENYDICQS
CFFSGRVAKGHKMHYPMVEYCTPTTSGEDVRDFAKVLKNKFRTKRYFAKHPRMGYLPVQTVLEGDNMETPVTLIN
FWPVDSAPASSPQLSHDDTHSRIEHYASRLAEMENSNGSYLNDSISPNESIDDEHLLIQHYCQSLNQDSPLSQPR
S
Utrophin N-terminus through R2_Dystrophin R23 to end of 1st
Coiled Coil:
(SEQ ID NO: 99)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EIAASASSDQWKRLHLSLQELLVWLQLKDDELSRQAPIGGDFPAVQKQNDVHRAFKRELKTKEPVIMSTLETVRI
FLTEQPLEGLEKLYQEPRELPPEERAQNVTRLLRKQAEEVNTEWEKLNLHSADWQRKIDETLERLQELQEATDEL
DLKLRQAEVIKGSWQPVGDLLIDSLQDHLEKVKALRGEIAPLKENVSHVNDLARQLTTLGIQLSPYNLSTLEDLN
TRWKLLQVAVEDRVRQLHEAHRDFGPASQHELSTSVQGPWERAISPNKVPYYINHETQTTCWDHPKMTELYQSLA
DLNNVRFSAYRTAMKLRRLQKALCLDLLSLSAACDALDQHNLKQNDQPMDILQIINCLTTIYDRLEQEHNNLVNV
PLCVDMCLNWLLNVYDTGRIGRIRVLSFKTGIISLCKAHLEDKYRYLFKQVASSTGFCDQRRLGLLLHDSIQIPR
QLGEVASFGGSNIEPSVRSCFQFANNKPEIEAALFLDWMRLEPQSMVWLPVLHRVAAAETAKHQAKCNICKECPI
IGFRYRSLKHFNYDICQSCFFSGRVAKGHKMHYPMVEYCTPTTSGEDVRDFAKVLKNKFRTKRYFAKHPRMGYLP
VQTVLEGDNMETPVTLINFWPVDSAPASSPQLSHDDTHSRIEHYASRLAEMENSNGSYLNDSISPNESIDDEHLL
IQHYCQSLNQDSPLSQPRSPAQILISLESEERGELERILADLEEENRNLQAEYDRLKQQHEHKGLSPLPSPPEMM
PTSPQSP
Utrophin N-terminus through R3_Dystrophin R23+R24+H4 to end of
syntrophin region:
(SEQ ID NO: 100)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAAASASSDQWKRLHLSLQELLVWLQLKDDELSRQAPIG
GDFPAVQKQNDVHRAFKRELKTKEPVIMSTLETVRIFLTEQPLEGLEKLYQEPRELPPEERAQNVTRLLRKQAEE
VNTEWEKLNLHSADWQRKIDETLERLQELQEATDELDLKLRQAEVIKGSWQPVGDLLIDSLQDHLEKVKALRGEI
APLKENVSHVNDLARQLTTLGIQLSPYNLSTLEDLNTRWKLLQVAVEDRVRQLHEAHRDFGPASQHELSTSVQGP
WERAISPNKVPYYINHETQTTCWDHPKMTELYQSLADLNNVRESAYRTAMKLRRLQKALCLDLLSLSAACDALDQ
HNLKQNDQPMDILQIINCLTTIYDRLEQEHNNLVNVPLCVDMCLNWLLNVYDTGRIGRIRVLSFKTGIISLCKAH
LEDKYRYLFKQVASSTGFCDQRRLGLLLHDSIQIPRQLGEVASFGGSNIEPSVRSCFQFANNKPEIEAALFLDWM
RLEPQSMVWLPVLHRVAAAETAKHQAKCNICKECPIIGFRYRSLKHFNYDICQSCFFSGRVAKGHKMHYPMVEYC
TPTTSGEDVRDFAKVLKNKFRTKRYFAKHPRMGYLPVQTVLEGDNMETPVTLINFWPVDSAPASSPQLSHDDTHS
RIEHYASRLAEMENSNGSYLNDSISPNESIDDEHLLIQHYCQSLNQDSPLSQPRS
Utrophin N-terminus through R2_Dystrophin R23 to end:
(SEQ ID NO: 101)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EIAASASSDQWKRLHLSLQELLVWLQLKDDELSRQAPIGGDFPAVQKQNDVHRAFKRELKTKEPVIMSTLETVRI
FLTEQPLEGLEKLYQEPRELPPEERAQNVTRLLRKQAEEVNTEWEKLNLHSADWQRKIDETLERLQELQEATDEL
DLKLRQAEVIKGSWQPVGDLLIDSLQDHLEKVKALRGEIAPLKENVSHVNDLARQLTTLGIQLSPYNLSTLEDLN
TRWKLLQVAVEDRVRQLHEAHRDFGPASQHFLSTSVQGPWERAISPNKVPYYINHETQTTCWDHPKMTELYQSLA
DLNNVRFSAYRTAMKLRRLQKALCLDLLSLSAACDALDQHNLKQNDQPMDILQIINCLTTIYDRLEQEHNNLVNV
PLCVDMCLNWLLNVYDTGRTGRIRVLSFKTGIISLCKAHLEDKYRYLFKQVASSTGFCDQRRLGLLLHDSIQIPR
QLGEVASFGGSNIEPSVRSCFQFANNKPEIEAALFLDWMRLEPQSMVWLPVLHRVAAAETAKHQAKCNICKECPI
IGFRYRSLKHFNYDICQSCFFSGRVAKGHKMHYPMVEYCTPTTSGEDVRDFAKVLKNKFRTKRYFAKHPRMGYLP
VQTVLEGDNMETPVTLINFWPVDSAPASSPQLSHDDTHSRIEHYASRLAEMENSNGSYLNDSISPNESIDDEHLL
IQHYCQSLNQDSPLSQPRSPAQILISLESEERGELERILADLEEENRNLQAEYDRLKQQHEHKGLSPLPSPPEMM
PTSPQSPRDAELIAEAKLLRQHKGRLEARMQILEDHNKQLESQLHRLRQLLEQPQAEAKVNGTTVSSPSTSLQRS
DSSQPMLLRVVGSQTSDSMGEEDLLSPPQDTSTGLEEVMEQLNNSFPSSRGRNTPGKPMREDTM
Utrophin N-terminus through R3; Dystrophin R24 to end:
(SEQ ID NO: 149)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAAASAQRKIDETLERLQELQEATDELDLKLRQAEVIKG
SWQPVGDLLIDSLQDHLEKVKALRGEIAPLKENVSHVNDLARQLTTLGIQLSPYNLSTLEDLNTRWKLLQVAVED
RVRQLHEAHRDFGPASQHELSTSVQGPWERAISPNKVPYYINHETQTTCWDHPKMTELYQSLADLNNVRFSAYRT
AMKLRRLQKALCLDLLSLSAACDALDQHNLKQNDQPMDILQIINCLTTIYDRLEQEHNNLVNVPLCVDMCLNWLL
NVYDTGRTGRIRVLSFKTGIISLCKAHLEDKYRYLFKQVASSTGFCDQRRLGLLLHDSIQIPRQLGEVASEGGSN
IEPSVRSCFQFANNKPEIEAALFLDWMRLEPQSMVWLPVLHRVAAAETAKHQAKCNICKECPIIGFRYRSLKHEN
YDICQSCFFSGRVAKGHKMHYPMVEYCTPTTSGEDVRDFAKVLKNKFRTKRYFAKHPRMGYLPVQTVLEGDNMET
PVTLINFWPVDSAPASSPQLSHDDTHSRIEHYASRLAEMENSNGSYLNDSISPNESIDDEHLLIQHYCQSLNQDS
PLSQPRSPAQILISLESEERGELERILADLEEENRNLQAEYDRLKQQHEHKGLSPLPSPPEMMPTSPQSPRDAEL
IAEAKLLRQHKGRLEARMQILEDHNKQLESQLHRLRQLLEQPQAEAKVNGTTVSSPSTSLQRSDSSQPMLLRVVG
SQTSDSMGEEDLLSPPQDTSTGLEEVMEQLNNSFPSSRGRNTPGKPMREDTM
Utrophin N-terminus through R3; Dystrophin R24 to end of
Syntrophin binding sites:
(SEQ ID NO: 150)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAAASAQRKIDETLERLQELQEATDELDLKLRQAEVIKG
SWQPVGDLLIDSLQDHLEKVKALRGEIAPLKENVSHVNDLARQLTTLGIQLSPYNLSTLEDLNTRWKLLQVAVED
RVRQLHEAHRDFGPASQHELSTSVQGPWERAISPNKVPYYINHETQTTCWDHPKMTELYQSLADLNNVRESAYRT
AMKLRRLQKALCLDLLSLSAACDALDQHNLKQNDQPMDILQIINCLTTIYDRLEQEHNNLVNVPLCVDMCLNWLL
NVYDTGRIGRIRVLSFKTGIISLCKAHLEDKYRYLFKQVASSTGFCDQRRLGLLLHDSIQIPRQLGEVASFGGSN
IEPSVRSCFQFANNKPEIEAALFLDWMRLEPQSMVWLPVLHRVAAAETAKHQAKCNICKECPIIGFRYRSLKHEN
YDICQSCFFSGRVAKGHKMHYPMVEYCTPTTSGEDVRDFAKVLKNKFRTKRYFAKHPRMGYLPVQTVLEGDNMET
PVTLINFWPVDSAPASSPQLSHDDTHSRIEHYASRLAEMENSNGSYLNDSISPNESIDDEHLLIQHYCQSLNQDS
PLSQPRS
Utrophin N-terminus through R3; Dystrophin R24 to end of 1st coiled
coil and proline- rich region:
(SEQ ID NO: 151)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAAASAQRKIDETLERLQELQEATDELDLKLRQAEVIKG
SWQPVGDLLIDSLQDHLEKVKALRGEIAPLKENVSHVNDLARQLTTLGIQLSPYNLSTLEDLNTRWKLLQVAVED
RVRQLHEAHRDFGPASQHELSTSVQGPWERAISPNKVPYYINHETQTTCWDHPKMTELYQSLADLNNVRFSAYRT
AMKLRRLQKALCLDLLSLSAACDALDQHNLKQNDQPMDILQIINCLTTIYDRLEQEHNNLVNVPLCVDMCLNWLL
NVYDTGRTGRIRVLSFKTGIISLCKAHLEDKYRYLFKQVASSTGFCDQRRLGLLLHDSIQIPRQLGEVASFGGSN
IEPSVRSCFQFANNKPEIEAALFLDWMRLEPQSMVWLPVLHRVAAAETAKHQAKCNICKECPIIGFRYRSLKHEN
YDICQSCFFSGRVAKGHKMHYPMVEYCTPTTSGEDVRDFAKVLKNKFRTKRYFAKHPRMGYLPVQTVLEGDNMET
PVTLINFWPVDSAPASSPQLSHDDTHSRIEHYASRLAEMENSNGSYLNDSISPNESIDDEHLLIQHYCQSLNQDS
PLSQPRSPAQILISLESEERGELERILADLEEENRNLQAEYDRLKQQHEHKGLSPLPSPPEMMPTSPQSP
Utrophin Nterm-H2_Dystrophin R24+H4 to end of syntrophin region:
(SEQ ID NO: 152)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAVAKLGMSQIPQKDLLETVRVREQAITKKSKQEAASAQ
RKIDETLERLQELQEATDELDLKLRQAEVIKGSWQPVGDLLIDSLQDHLEKVKALRGEIAPLKENVSHVNDLARQ
LTTLGIQLSPYNLSTLEDLNTRWKLLQVAVEDRVRQLHEAHRDFGPASQHELSTSVQGPWERAISPNKVPYYINH
ETQTTCWDHPKMTELYQSLADLNNVRFSAYRTAMKLRRLQKALCLDLLSLSAACDALDQHNLKQNDQPMDILQII
NCLTTIYDRLEQEHNNLVNVPLCVDMCLNWLLNVYDTGRTGRIRVLSFKTGIISLCKAHLEDKYRYLFKQVASST
GFCDQRRLGLLLHDSIQIPRQLGEVASFGGSNIEPSVRSCFQFANNKPEIEAALFLDWMRLEPQSMVWLPVLHRV
AAAETAKHQAKCNICKECPIIGFRYRSLKHFNYDICQSCFFSGRVAKGHKMHYPMVEYCTPTTSGEDVRDFAKVL
KNKFRTKRYFAKHPRMGYLPVQTVLEGDNMETPVTLINFWPVDSAPASSPQLSHDDTHSRIEHYASRLAEMENSN
GSYLNDSISPNESIDDEHLLIQHYCQSLNQDSPLSQPRS
Utrophin N-terminus through H2; Dystrophin R24 to end:
(SEQ ID NO: 153)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAVAKLGMSQIPQKDLLETVRVREQAITKKSKQEAASAQ
RKIDETLERLQELQEATDELDLKLRQAEVIKGSWQPVGDLLIDSLQDHLEKVKALRGEIAPLKENVSHVNDLARQ
LTTLGIQLSPYNLSTLEDLNTRWKLLQVAVEDRVRQLHEAHRDFGPASQHELSTSVQGPWERAISPNKVPYYINH
ETQTTCWDHPKMTELYQSLADLNNVRFSAYRTAMKLRRLQKALCLDLLSLSAACDALDQHNLKQNDQPMDILQII
NCLTTIYDRLEQEHNNLVNVPLCVDMCLNWLLNVYDTGRTGRIRVLSFKTGIISLCKAHLEDKYRYLFKQVASST
GFCDQRRLGLLLHDSIQIPRQLGEVASFGGSNIEPSVRSCFQFANNKPEIEAALFLDWMRLEPQSMVWLPVLHRV
AAAETAKHQAKCNICKECPIIGFRYRSLKHFNYDICQSCFFSGRVAKGHKMHYPMVEYCTPTTSGEDVRDFAKVL
KNKFRTKRYFAKHPRMGYLPVQTVLEGDNMETPVTLINFWPVDSAPASSPQLSHDDTHSRIEHYASRLAEMENSN
GSYLNDSISPNESIDDEHLLIQHYCQSLNQDSPLSQPRSPAQILISLESEERGELERILADLEEENRNLQAEYDR
LKQQHEHKGLSPLPSPPEMMPTSPQSPRDAELIAEAKLLRQHKGRLEARMQILEDHNKQLESQLHRLRQLLEQPQ
AEAKVNGTTVSSPSTSLQRSDSSQPMLLRVVGSQTSDSMGEEDLLSPPQDTSTGLEEVMEQLNNSFPSSRGRNTP
GKPMREDTM
Utrophin N-terminus through R3; Dystrophin R23 to end:
(SEQ ID NO: 154)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMTLLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAAASASSDQWKRLHLSLQELLVWLQLKDDELSRQAPIG
GDFPAVQKQNDVHRAFKRELKTKEPVIMSTLETVRIFLTEQPLEGLEKLYQEPRELPPEERAQNVTRLLRKQAEE
VNTEWEKLNLHSADQRKIDETLERLQELQEATDELDLKLRQAEVIKGSWQPVGDLLIDSLQDHLEKVKALRGEIA
PLKENVSHVNDLARQLTTLGIQLSPYNLSTLEDLNTRWKLLQVAVEDRVRQLHEAHRDFGPASQHELSTSVQGPW
ERAISPNKVPYYINHETQTTCWDHPKMTELYQSLADLNNVRESAYRTAMKLRRLQKALCLDLLSLSAACDALDQH
NLKQNDQPMDILQIINCLTTIYDRLEQEHNNLVNVPLCVDMCLNWLLNVYDTGRTGRIRVLSFKTGIISLCKAHL
EDKYRYLFKQVASSTGFCDQRRLGLLLHDSIQIPRQLGEVASFGGSNIEPSVRSCFQFANNKPEIEAALFLDWMR
LEPQSMVWLPVLHRVAAAETAKHQAKCNICKECPIIGFRYRSLKHFNYDICQSCFFSGRVAKGHKMHYPMVEYCT
PTTSGEDVRDFAKVLKNKFRTKRYFAKHPRMGYLPVQTVLEGDNMETPVTLINFWPVDSAPASSPQLSHDDTHSR
IEHYASRLAEMENSNGSYLNDSISPNESIDDEHLLIQHYCQSLNQDSPLSQPRSPAQILISLESEERGELERILA
DLEEENRNLQAEYDRLKQQHEHKGLSPLPSPPEMMPTSPQSPRDAELIAEAKLLRQHKGRLEARMQILEDHNKQL
ESQLHRLRQLLEQPQAEAKVNGTTVSSPSTSLQRSDSSQPMLLRVVGSQTSDSMGEEDLLSPPQDTSTGLEEVME
QLNNSFPSSRGRNTPGKPMREDTM
Utrophin Nterm-R3_Dystrophin R23+H4 to to 1st coiled coil and
proline rich region that follows:
(SEQ ID NO: 155)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFATHEAFMMELTAHQSSVGSVLQAGNQ
LITQGTLSDEEEFEIQEQMILLNARWEALRVESMDRQSRLHDVLMELQKKQLQQLSAWLTLTEERIQKMETCPLD
DDVKSLQKLLEEHKSLQSDLEAEQVKVNSLTHMVVIVDENSGESATAILEDQLQKLGERWTAVCRWTEERWNRLQ
EINILWQELLEEQCLLKAWLTEKEEALNKVQTSNFKDQKELSVSVRRLAILKEDMEMKRQTLDQLSEIGQDVGQL
LDNSKASKKINSDSEELTQRWDSLVQRLEDSSNQVTQAAASASSDQWKRLHLSLQELLVWLQLKDDELSRQAPIG
GDFPAVQKQNDVHRAFKRELKTKEPVIMSTLETVRIFLTEQPLEGLEKLYQEPRELPPEERAQNVTRLLRKQAEE
VNTEWEKLNLHSADQRKIDETLERLQELQEATDELDLKLRQAEVIKGSWQPVGDLLIDSLQDHLEKVKALRGEIA
PLKENVSHVNDLARQLTTLGIQLSPYNLSTLEDLNTRWKLLQVAVEDRVRQLHEAHRDFGPASQHELSTSVQGPW
ERAISPNKVPYYINHETQTTCWDHPKMTELYQSLADLNNVRFSAYRTAMKLRRLQKALCLDLLSLSAACDALDQH
NLKQNDQPMDILQIINCLTTIYDRLEQEHNNLVNVPLCVDMCLNWLLNVYDTGRIGRIRVLSFKTGIISLCKAHL
EDKYRYLFKQVASSTGFCDQRRLGLLLHDSIQIPRQLGEVASFGGSNIEPSVRSCFQFANNKPEIEAALFLDWMR
LEPQSMVWLPVLHRVAAAETAKHQAKCNICKECPIIGFRYRSLKHFNYDICQSCFFSGRVAKGHKMHYPMVEYCT
PTTSGEDVRDFAKVLKNKFRTKRYFAKHPRMGYLPVQTVLEGDNMETPVTLINFWPVDSAPASSPQLSHDDTHSR
IEHYASRLAEMENSNGSYLNDSISPNESIDDEHLLIQHYCQSLNQDSPLSQPRSPAQILISLESEERGELERILA
DLEEENRNLQAEYDRLKQQHEHKGLSPLPSPPEMMPTSPQSP

### Components of the Disclosed Chimeric Constructs

### Micro-Dystrophins

The disclosure also provides nucleic acids encoding any of the micro-dystrophins optimized for cardiac muscle described herein. Such nucleic acids may be DNA or RNA molecules. These nucleic acids may be used, for example, in methods for making micro-dystrophins or as direct therapeutic agents in a gene therapy approach. A micro-dystrophin protein optimized for cardiac muscle is further understood to include proteins that are variants of any one of SEQ ID Nos. 1-6, 80-84, and 120-125. Variant polypeptides include polypeptides that differ by one or more (e.g., 1, 2, 3, 4, 5, 6, 7, 8, 9, 10, 11, 12, 13, 14, 15, 16, 17, 18, 19, 20, or more) amino acid substitutions, additions, or deletions, and will, therefore, include amino acid sequences that differ from the amino acid sequences designated in any one of SEQ ID Nos. 1-6, 80-84, and 120-125.

In some embodiments, a micro-dystrophin protein optimized for cardiac muscle comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 1-6, 80-84, and 120-125.

In some embodiments, a micro-dystrophin protein optimized for cardiac muscle consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 1-6, 80-84, and 120-125.

In some embodiments, a micro-dystrophin protein optimized for cardiac muscle consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 1-6, 80-84, and 120-125.

In some embodiments, functional variants or modified forms of micro-dystrophin proteins optimized for cardiac muscle include fusion proteins having at least a portion of the micro-dystrophin protein and one or more fusion domains. Well-known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. Other fusion domains are particularly useful for increasing protein stability.

Non-limiting examples of nucleotide sequences encoding human micro-dystrophin proteins optimized for cardiac muscle are described below.

Nterm-R1_R17-R19_R24+H4+trCterm:
(SEQ ID NO: 7)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCGATTTGACAGATCTGTTGAGAAATGGCGGCGTTTTCATTATGATATAAAGATATTTAATCAGTGGCTAACA
GAAGCTGAACAGTTTCTCAGAAAGACACAAATTCCTGAGAATTGGGAACATGCTAAATACAAATGGTATCTTAAG
GAACTCCAGGATGGCATTGGGCAGCGGCAAACTGTTGTCAGAACATTGAATGCAACTGGGGAAGAAATAATTCAG
CAATCCTCAAAAACAGATGCCAGTATTCTACAGGAAAAATTGGGAAGCCTGAATCTGCGGTGGCAGGAGGTCTGC
AAACAGCTGTCAGACAGAAAAAAGAGGCTAGAAGAACAAAAGAATATCTTGTCAGAATTTCAAAGAGATTTAAAT
GAATTTGTTTTATGGTTGGAGGAAGCAGATAACATTGCTAGTATCCCACTTGAACCTGGAAAAGAGCAGCAACTA
AAAGAAAAGCTTGAGCAAGTCAAGTTACTGGTGGAAGAGTTGCCCCTGCGCCAGGGAATTCTCAAACAATTAAAT
GAAACTGGAGGACCCGTGCTTGTAAGTGCTCCCATAAGCCCAGAAGAGCAAGATAAACTTGAAAATAAGCTCAAG
CAGACAAATCTCCAGTGGATAAAGGTTTCCAGAGCTTTACCTGAGAAACAAGGAGAAATTGAAGCTCAAATAAAA
GACCTTGGGCAGCTTGAAAAAAAGCTTGAAGACCTTGAAGAGCAGTTAAATCATCTGCTGCTGTGGTTATCTCCT
ATTAGGAATCAGTTGGAAATTTATAACCAACCAAACCAAGAAGGACCATTTGACGTTAAGGAAACTGAAATAGCA
GTTCAAGCTAAACAACCGGATGTGGAAGAGATTTTGTCTAAAGGGCAGCATTTGTACAAGGAAAAACCAGCCACT
CAGCCAGTGAAGAGGAAGTTAGAAGATCTGAGCTCTGAGTGGAAGGCGGTAAACCGTTTACTTCAAGAGCTGAGG
GCAAAGCAGTGGCAGAGAAAAATAGATGAGACCCTTGAAAGACTCCGGGAACTTCAAGAGGCCACGGATGAGCTG
GACCTCAAGCTGCGCCAAGCTGAGGTGATCAAGGGATCCTGGCAGCCCGTGGGCGATCTCCTCATTGACTCTCTC
CAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAAC
ACCAGATGGAAGCTTCTGCAGGTGGCCGTCGAGGACCGAGTCAGGCAGCTGCATGAAGCCCACAGGGACTTTGGT
CCAGCATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTTTAGCT
GACCTGAATAATGTCAGATTCTCAGCTTATAGGACTGCCATGAAACTCCGAAGACTGCAGAAGGCCCTTTGCTTG
GATCTCTTGAGCCTGTCAGCTGCATGTGATGCCTTGGACCAGCACAACCTCAAGCAAAATGACCAGCCCATGGAT
ATCCTGCAGATTATTAATTGTTTGACCACTATTTATGACCGCCTGGAGCAAGAGCACAACAATTTGGTCAACGTC
CCTCTCTGCGTGGATATGTGTCTGAACTGGCTGCTGAATGTTTATGATACGGGACGAACAGGGAGGATCCGTGTC
CTGTCTTTTAAAACTGGCATCATTTCCCTGTGTAAAGCACATTTGGAAGACAAGTACAGATACCTTTTCAAGCAA
GTGGCAAGTTCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCC
GTCCTGCACAGAGTGGCTGCTGCAGAAACTGCCAAGCATCAGGCCAAATGTAACATCTGCAAAGAGTGTCCAATC
ATTGGATTCAGGTACAGGAGTCTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTACATCAGGAGAAGATGTTCGAGAC
TTTGCCAAGGTACTAAAAAACAAATTTCGAACCAAAAGGTATTTTGCGAAGCATCCCCGAATGGGCTACCTGCCA
GTGCAGACTGTCTTAGAGGGGGACAACATGGAAACTCCC
Nterm-R1_R17-R19_R24+H4+trCterm:
(SEQ ID NO: 126)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATAGATCTGTTGAGAAATGGCGGCGTTTTCATTATGATATAAAGATATTTAATCAGTGGCTAACAGAAGCTGAA
CAGTTTCTCAGAAAGACACAAATTCCTGAGAATTGGGAACATGCTAAATACAAATGGTATCTTAAGGAACTCCAG
GATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTG
TCAGACAGAAAAAAGAGGCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGCAGCAACTAAAAGAAAAG
CTTGAGCAAGTCAAGTTACTGGTGGAAGAGTTGCCCCTGCGCCAGGGAATTCTCAAACAATTAAATGAAACTGGA
GGACCCGTGCTTGTAAGTGCTCCCATAAGCCCAGAAGAGCAAGATAAACTTGAAAATAAGCTCAAGCAGACAAAT
CTCCAGTGGATAAAGGTTTCCAGAGCTTTACCTGAGAAACAAGGAGAAATTGAAGCTCAAATAAAAGACCTTGGG
CAGCTTGAAAAAAAGCTTGAAGACCTTGAAGAGCAGTTAAATCATCTGCTGCTGTGGTTATCTCCTATTAGGAAT
CAGTTGGAAATTTATAACCAACCAAACCAAGAAGGACCATTIGACGTTAAGGAAACTGAAATAGCAGTTCAAGCT
AAACAACCGGATGTGGAAGAGATTTTGTCTAAAGGGCAGCATTTGTACAAGGAAAAACCAGCCACTCAGCCAGTG
AAGAGGAAGTTAGAAGATCTGAGCTCTGAGTGGAAGGCGGTAAACCGTTTACTTCAAGAGCTGAGGGCAAAGCAG
TGGCAGAGAAAAATAGATGAGACCCTTGAAAGACTCCGGGAACTTCAAGAGGCCACGGATGAGCTGGACCTCAAG
CTGCGCCAAGCTGAGGTGATCAAGGGATCCTGGCAGCCCGTGGGCGATCTCCTCATTGACTCTCTCCAAGATCAC
CTCGAGAAAGTCAAGGCACTTCGAGGAGAAATTGCGCCTCTGAAAGAGAACGTGAGCCACGTCAATGACCTTGCT
CGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTTGGTCCAGCATCT
CAGCACTTTCTTTCCACGTCTGTCCAGGGTCCCTGGGAGAGAGCCATCTCGCCAAACAAAGTGCCCTACTATATC
AACCACGAGACTCAAACAACTTGCTGGGACCATCCCAAAATGACAGAGCTCTACCAGTCTTTAGCTGACCTGAAT
AATGTCAGATTCTCAGCTTATAGGACTGCCATGAAACTCCGAAGACTGCAGAAGGCCCTTTGCTTGGATCTCTTG
AGCCTGTCAGCTGCATGTGATGCCTTGGACCAGCACAACCTCAAGCAAAATGACCAGCCCATGGATATCCTGCAG
ATTATTAATTGTTTGACCACTATTTATGACCGCCTGGAGCAAGAGCACAACAATTTGGTCAACGTCCCTCTCTGC
GTGGATATGTGTCTGAACTGGCTGCTGAATGTTTATGATACGGGACGAACAGGGAGGATCCGTGTCCTGTCTTTT
AAAACTGGCATCATTTCCCTGTGTAAAGCACATTTGGAAGACAAGTACAGATACCTTTTCAAGCAAGTGGCAAGT
TCAACAGGATTTTGTGACCAGCGCAGGCTGGGCCTCCTTCTGCATGATTCTATCCAAATTCCAAGACAGTTGGGT
GAAGTTGCATCCTTTGGGGGCAGTAACATTGAGCCAAGTGTCCGGAGCTGCTTCCAATTTGCTAATAATAAGCCA
GAGATCGAAGCGGCCCTCTTCCTAGACTGGATGAGACTGGAACCCCAGTCCATGGTGTGGCTGCCCGTCCTGCAC
AGAGTGGCTGCTGCAGAAACTGCCAAGCATCAGGCCAAATGTAACATCTGCAAAGAGTGTCCAATCATTGGATTC
AGGTACAGGAGTCTAAAGCACTTTAATTATGACATCTGCCAAAGCTGCTTTTTTTCTGGTCGAGTTGCAAAAGGC
CATAAAATGCACTATCCCATGGTGGAATATTGCACTCCGACTACATCAGGAGAAGATGTTCGAGACTTTGCCAAG
GTACTAAAAAACAAATTTCGAACCAAAAGGTATTTTGCGAAGCATCCCCGAATGGGCTACCTGCCAGTGCAGACT
GTCTTAGAGGGGGACAACATGGAAACTCCC
Nterm-R1_R17-R19_H4 to 1st coiled coil and proline rich
region that follows:
(SEQ ID NO: 8)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCGATTTGACAGATCTGTTGAGAAATGGCGGCGTTTTCATTATGATATAAAGATATTTAATCAGTGGCTAACA
GAAGCTGAACAGTTTCTCAGAAAGACACAAATTCCTGAGAATTGGGAACATGCTAAATACAAATGGTATCTTAAG
GAACTCCAGGATGGCATTGGGCAGCGGCAAACTGTTGTCAGAACATTGAATGCAACTGGGGAAGAAATAATTCAG
CAATCCTCAAAAACAGATGCCAGTATTCTACAGGAAAAATTGGGAAGCCTGAATCTGCGGTGGCAGGAGGTCTGC
AAACAGCTGTCAGACAGAAAAAAGAGGCTAGAAGAACAAAAGAATATCTTGTCAGAATTTCAAAGAGATTTAAAT
GAATTTGTTTTATGGTTGGAGGAAGCAGATAACATTGCTAGTATCCCACTTGAACCTGGAAAAGAGCAGCAACTA
AAAGAAAAGCTTGAGCAAGTCAAGTTACTGGTGGAAGAGTTGCCCCTGCGCCAGGGAATTCTCAAACAATTAAAT
GAAACTGGAGGACCCGTGCTTGTAAGTGCTCCCATAAGCCCAGAAGAGCAAGATAAACTTGAAAATAAGCTCAAG
CAGACAAATCTCCAGTGGATAAAGGTTTCCAGAGCTTTACCTGAGAAACAAGGAGAAATTGAAGCTCAAATAAAA
GACCTTGGGCAGCTTGAAAAAAAGCTTGAAGACCTTGAAGAGCAGTTAAATCATCTGCTGCTGTGGTTATCTCCT
ATTAGGAATCAGTTGGAAATTTATAACCAACCAAACCAAGAAGGACCATTTGACGTTAAGGAAACTGAAATAGCA
GTTCAAGCTAAACAACCGGATGTGGAAGAGATTTTGTCTAAAGGGCAGCATTTGTACAAGGAAAAACCAGCCACT
CAGCCAGTGAAGAGGAAGTTAGAAGATCTGAGCTCTGAGTGGAAGGCGGTAAACCGTTTACTTCAAGAGCTGAGG
GCAAAGCAGGGTCCAGCATCTCAGCACTTTCTTTCCACGTCTGTCCAGGGTCCCTGGGAGAGAGCCATCTCGCCA
AACAAAGTGCCCTACTATATCAACCACGAGACTCAAACAACTTGCTGGGACCATCCCAAAATGACAGAGCTCTAC
CAGTCTTTAGCTGACCTGAATAATGTCAGATTCTCAGCTTATAGGACTGCCATGAAACTCCGAAGACTGCAGAAG
GCCCTTTGCTTGGATCTCTTGAGCCTGTCAGCTGCATGTGATGCCTTGGACCAGCACAACCTCAAGCAAAATGAC
CAGCCCATGGATATCCTGCAGATTATTAATTGTTTGACCACTATTTATGACCGCCTGGAGCAAGAGCACAACAAT
TTGGTCAACGTCCCTCTCTGCGTGGATATGTGTCTGAACTGGCTGCTGAATGTTTATGATACGGGACGAACAGGG
AGGATCCGTGTCCTGTCTTTTAAAACTGGCATCATTTCCCTGTGTAAAGCACATTTGGAAGACAAGTACAGATAC
CTTTTCAAGCAAGTGGCAAGTTCAACAGGATTTTGTGACCAGCGCAGGCTGGGCCTCCTTCTGCATGATTCTATC
CAAATTCCAAGACAGTTGGGTGAAGTTGCATCCTTTGGGGGCAGTAACATTGAGCCAAGTGTCCGGAGCTGCTTC
CAATTTGCTAATAATAAGCCAGAGATCGAAGCGGCCCTCTTCCTAGACTGGATGAGACTGGAACCCCAGTCCATG
GTGTGGCTGCCCGTCCTGCACAGAGTGGCTGCTGCAGAAACTGCCAAGCATCAGGCCAAATGTAACATCTGCAAA
GAGTGTCCAATCATTGGATTCAGGTACAGGAGTCTAAAGCACTTTAATTATGACATCTGCCAAAGCTGCTTTTTT
TCTGGTCGAGTTGCAAAAGGCCATAAAATGCACTATCCCATGGTGGAATATTGCACTCCGACTACATCAGGAGAA
GATGTTCGAGACTTTGCCAAGGTACTAAAAAACAAATTTCGAACCAAAAGGTATTTTGCGAAGCATCCCCGAATG
GGCTACCTGCCAGTGCAGACTGTCTTAGAGGGGGACAACATGGAAACTCCCGTTACTCTGATCAACTTCTGGCCA
GTAGATTCTGCGCCTGCCTCGTCCCCTCAGCTTTCACACGATGATACTCATTCACGCATTGAACATTATGCTAGC
AGGCTAGCAGAAATGGAAAACAGCAATGGATCTTATCTAAATGATAGCATCTCTCCTAATGAGAGCATAGATGAT
GAACATTTGTTAATCCAGCATTACTGCCAAAGTTTGAACCAGGACTCCCCCCTGAGCCAGCCTCGTAGTCCTGCC
CAGATCTTGATTTCCTTAGAGAGTGAGGAAAGAGGGGAGCTAGAGAGAATCCTAGCAGATCTTGAGGAAGAAAAC
AGGAATCTGCAAGCAGAATATGACCGTCTAAAGCAGCAGCACGAACATAAAGGCCTGTCCCCACTGCCGTCCCCT
CCTGAAATGATGCCCACCTCTCCCCAGAGTCCC
Nterm-R1_R17-R19_H4 to 1st coiled coil and proline rich
region that follows:
(SEQ ID NO: 127)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATAGATCTGTTGAGAAATGGCGGCGTTTTCATTATGATATAAAGATATTTAATCAGTGGCTAACAGAAGCTGAA
CAGTTTCTCAGAAAGACACAAATTCCTGAGAATTGGGAACATGCTAAATACAAATGGTATCTTAAGGAACTCCAG
GATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTG
TCAGACAGAAAAAAGAGGCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGCAGCAACTAAAAGAAAAG
CTTGAGCAAGTCAAGTTACTGGTGGAAGAGTTGCCCCTGCGCCAGGGAATTCTCAAACAATTAAATGAAACTGGA
GGACCCGTGCTTGTAAGTGCTCCCATAAGCCCAGAAGAGCAAGATAAACTTGAAAATAAGCTCAAGCAGACAAAT
CTCCAGTGGATAAAGGTTTCCAGAGCTTTACCTGAGAAACAAGGAGAAATTGAAGCTCAAATAAAAGACCTTGGG
CAGCTTGAAAAAAAGCTTGAAGACCTTGAAGAGCAGTTAAATCATCTGCTGCTGTGGTTATCTCCTATTAGGAAT
CAGTTGGAAATTTATAACCAACCAAACCAAGAAGGACCATTTGACGTTAAGGAAACTGAAATAGCAGTTCAAGCT
AAACAACCGGATGTGGAAGAGATTTTGTCTAAAGGGCAGCATTTGTACAAGGAAAAACCAGCCACTCAGCCAGTG
AAGAGGAAGTTAGAAGATCTGAGCTCTGAGTGGAAGGCGGTAAACCGTTTACTTCAAGAGCTGAGGGCAAAGCAG
GGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTA
GCTGACCTGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAACAATTTGGTCAAC
GTCCCTCTCTGCGTGGATATGTGTCTGAACTGGCTGCTGAATGTTTATGATACGGGACGAACAGGGAGGATCCGT
GTCCTGTCTTTTAAAACTGGCATCATTTCCCTGTGTAAAGCACATTTGGAAGACAAGTACAGATACCTTTTCAAG
CAAGTGGCAAGTTCAACAGGATTTTGTGACCAGCGCAGGCTGGGCCTCCTTCTGCATGATTCTATCCAAATTCCA
AGACAGTTGGGTGAAGTTGCATCCTTTGGGGGCAGTAACATTGAGCCAAGTGTCCGGAGCTGCTTCCAATTTGCT
AATAATAAGCCAGAGATCGAAGCGGCCCTCTTCCTAGACTGGATGAGACTGGAACCCCAGTCCATGGTGTGGCTG
CCCGTCCTGCACAGAGTGGCTGCTGCAGAAACTGCCAAGCATCAGGCCAAATGTAACATCTGCAAAGAGTGTCCA
ATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGA
GACTTTGCCAAGGTACTAAAAAACAAATTTCGAACCAAAAGGTATTTTGCGAAGCATCCCCGAATGGGCTACCTG
CCAGTGCAGACTGTCTTAGAGGGGGACAACATGGAAACTCCCGTTACTCTGATCAACTTCTGGCCAGTAGATTCT
GCGCCTGCCTCGTCCCCTCAGCTTTCACACGATGATACTCATTCACGCATTGAACATTATGCTAGCAGGCTAGCA
GAAATGGAAAACAGCAATGGATCTTATCTAAATGATAGCATCTCTCCTAATGAGAGCATAGATGATGAACATTTG
TTAATCCAGCATTACTGCCAAAGTTTGAACCAGGACTCCCCCCTGAGCCAGCCTCGTAGTCCTGCCCAGATCTTG
ATTTCCTTAGAGAGTGAGGAAAGAGGGGAGCTAGAGAGAATCCTAGCAGATCTTGAGGAAGAAAACAGGAATCTG
CAAGCAGAATATGACCGTCTAAAGCAGCAGCACGAACATAAAGGCCTGTCCCCACTGCCGTCCCCTCCTGAAATG
ATGCCCACCTCTCCCCAGAGTCCC
Nterm-R1_R17-R19_H4 to end of syntrophin region:
(SEQ ID NO: 9)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCGATTTGACAGATCTGTTGAGAAATGGCGGCGTTTTCATTATGATATAAAGATATTTAATCAGTGGCTAACA
GAAGCTGAACAGTTTCTCAGAAAGACACAAATTCCTGAGAATTGGGAACATGCTAAATACAAATGGTATCTTAAG
GAACTCCAGGATGGCATTGGGCAGCGGCAAACTGTTGTCAGAACATTGAATGCAACTGGGGAAGAAATAATTCAG
CAATCCTCAAAAACAGATGCCAGTATTCTACAGGAAAAATTGGGAAGCCTGAATCTGCGGTGGCAGGAGGTCTGC
AAACAGCTGTCAGACAGAAAAAAGAGGCTAGAAGAACAAAAGAATATCTTGTCAGAATTTCAAAGAGATTTAAAT
GAATTTGTTTTATGGTTGGAGGAAGCAGATAACATTGCTAGTATCCCACTTGAACCTGGAAAAGAGCAGCAACTA
AAAGAAAAGCTTGAGCAAGTCAAGTTACTGGTGGAAGAGTTGCCCCTGCGCCAGGGAATTCTCAAACAATTAAAT
GAAACTGGAGGACCCGTGCTTGTAAGTGCTCCCATAAGCCCAGAAGAGCAAGATAAACTTGAAAATAAGCTCAAG
CAGACAAATCTCCAGTGGATAAAGGTTTCCAGAGCTTTACCTGAGAAACAAGGAGAAATTGAAGCTCAAATAAAA
GACCTTGGGCAGCTTGAAAAAAAGCTTGAAGACCTTGAAGAGCAGTTAAATCATCTGCTGCTGTGGTTATCTCCT
ATTAGGAATCAGTTGGAAATTTATAACCAACCAAACCAAGAAGGACCATTTGACGTTAAGGAAACTGAAATAGCA
GTTCAAGCTAAACAACCGGATGTGGAAGAGATTTTGTCTAAAGGGCAGCATTTGTACAAGGAAAAACCAGCCACT
CAGCCAGTGAAGAGGAAGTTAGAAGATCTGAGCTCTGAGTGGAAGGCGGTAAACCGTTTACTTCAAGAGCTGAGG
GCAAAGCAGGGTCCAGCATCTCAGCACTTTCTTTCCACGTCTGTCCAGGGTCCCTGGGAGAGAGCCATCTCGCCA
AACAAAGTGCCCTACTATATCAACCACGAGACTCAAACAACTTGCTGGGACCATCCCAAAATGACAGAGCTCTAC
CAGTCTTTAGCTGACCTGAATAATGTCAGATTCTCAGCTTATAGGACTGCCATGAAACTCCGAAGACTGCAGAAG
GCCCTTTGCTTGGATCTCTTGAGCCTGTCAGCTGCATGTGATGCCTTGGACCAGCACAACCTCAAGCAAAATGAC
CAGCCCATGGATATCCTGCAGATTATTAATTGTTTGACCACTATTTATGACCGCCTGGAGCAAGAGCACAACAAT
TTGGTCAACGTCCCTCTCTGCGTGGATATGTGTCTGAACTGGCTGCTGAATGTTTATGATACGGGACGAACAGGG
AGGATCCGTGTCCTGTCTTTTAAAACTGGCATCATTTCCCTGTGTAAAGCACATTTGGAAGACAAGTACAGATAC
CTTTTCAAGCAAGTGGCAAGTTCAACAGGATTTTGTGACCAGCGCAGGCTGGGCCTCCTTCTGCATGATTCTATC
CAAATTCCAAGACAGTTGGGTGAAGTTGCATCCTTTGGGGGCAGTAACATTGAGCCAAGTGTCCGGAGCTGCTTC
CAATTTGCTAATAATAAGCCAGAGATCGAAGCGGCCCTCTTCCTAGACTGGATGAGACTGGAACCCCAGTCCATG
GTGTGGCTGCCCGTCCTGCACAGAGTGGCTGCTGCAGAAACTGCCAAGCATCAGGCCAAATGTAACATCTGCAAA
GAGTGTCCAATCATTGGATTCAGGTACAGGAGTCTAAAGCACTTTAATTATGACATCTGCCAAAGCTGCTTTTTT
TCTGGTCGAGTTGCAAAAGGCCATAAAATGCACTATCCCATGGTGGAATATTGCACTCCGACTACATCAGGAGAA
GATGTTCGAGACTTTGCCAAGGTACTAAAAAACAAATTTCGAACCAAAAGGTATTTTGCGAAGCATCCCCGAATG
GGCTACCTGCCAGTGCAGACTGTCTTAGAGGGGGACAACATGGAAACTCCCGTTACTCTGATCAACTTCTGGCCA
GTAGATTCTGCGCCTGCCTCGTCCCCTCAGCTTTCACACGATGATACTCATTCACGCATTGAACATTATGCTAGC
AGGCTAGCAGAAATGGAAAACAGCAATGGATCTTATCTAAATGATAGCATCTCTCCTAATGAGAGCATAGATGAT
GAACATTTGTTAATCCAGCATTACTGCCAAAGTTTGAACCAGGACTCCCCCCTGAGCCAGCCTCGTAGT
Nterm-R1_R17-R19_H4 to end of syntrophin region:
(SEQ ID NO: 128)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATAGATCTGTTGAGAAATGGCGGCGTTTTCATTATGATATAAAGATATTTAATCAGTGGCTAACAGAAGCTGAA
CAGTTTCTCAGAAAGACACAAATTCCTGAGAATTGGGAACATGCTAAATACAAATGGTATCTTAAGGAACTCCAG
GATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTG
TCAGACAGAAAAAAGAGGCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGCAGCAACTAAAAGAAAAG
CTTGAGCAAGTCAAGTTACTGGTGGAAGAGTTGCCCCTGCGCCAGGGAATTCTCAAACAATTAAATGAAACTGGA
GGACCCGTGCTTGTAAGTGCTCCCATAAGCCCAGAAGAGCAAGATAAACTTGAAAATAAGCTCAAGCAGACAAAT
CTCCAGTGGATAAAGGTTTCCAGAGCTTTACCTGAGAAACAAGGAGAAATTGAAGCTCAAATAAAAGACCTTGGG
CAGCTTGAAAAAAAGCTTGAAGACCTTGAAGAGCAGTTAAATCATCTGCTGCTGTGGTTATCTCCTATTAGGAAT
CAGTTGGAAATTTATAACCAACCAAACCAAGAAGGACCATTTGACGTTAAGGAAACTGAAATAGCAGTTCAAGCT
AAACAACCGGATGTGGAAGAGATTTTGTCTAAAGGGCAGCATTTGTACAAGGAAAAACCAGCCACTCAGCCAGTG
AAGAGGAAGTTAGAAGATCTGAGCTCTGAGTGGAAGGCGGTAAACCGTTTACTTCAAGAGCTGAGGGCAAAGCAG
GGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTA
GCTGACCTGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAACAATTTGGTCAAC
GTCCCTCTCTGCGTGGATATGTGTCTGAACTGGCTGCTGAATGTTTATGATACGGGACGAACAGGGAGGATCCGT
GTCCTGTCTTTTAAAACTGGCATCATTTCCCTGTGTAAAGCACATTTGGAAGACAAGTACAGATACCTTTTCAAG
CAAGTGGCAAGTTCAACAGGATTTTGTGACCAGCGCAGGCTGGGCCTCCTTCTGCATGATTCTATCCAAATTCCA
AGACAGTTGGGTGAAGTTGCATCCTTTGGGGGCAGTAACATTGAGCCAAGTGTCCGGAGCTGCTTCCAATTTGCT
AATAATAAGCCAGAGATCGAAGCGGCCCTCTTCCTAGACTGGATGAGACTGGAACCCCAGTCCATGGTGTGGCTG
CCCGTCCTGCACAGAGTGGCTGCTGCAGAAACTGCCAAGCATCAGGCCAAATGTAACATCTGCAAAGAGTGTCCA
ATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGA
GACTTTGCCAAGGTACTAAAAAACAAATTTCGAACCAAAAGGTATTTTGCGAAGCATCCCCGAATGGGCTACCTG
CCAGTGCAGACTGTCTTAGAGGGGGACAACATGGAAACTCCCGTTACTCTGATCAACTTCTGGCCAGTAGATTCT
GCGCCTGCCTCGTCCCCTCAGCTTTCACACGATGATACTCATTCACGCATTGAACATTATGCTAGCAGGCTAGCA
GAAATGGAAAACAGCAATGGATCTTATCTAAATGATAGCATCTCTCCTAATGAGAGCATAGATGATGAACATTTG
TTAATCCAGCATTACTGCCAAAGTTTGAACCAGGACTCCCCCCTGAGCCAGCCTCGTAGT
Nterm-R1_R17-R19_R24 to 1st coiled coil and proline rich
region that follows:
(SEQ ID NO: 10)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCGATTTGACAGATCTGTTGAGAAATGGCGGCGTTTTCATTATGATATAAAGATATTTAATCAGTGGCTAACA
GAAGCTGAACAGTTTCTCAGAAAGACACAAATTCCTGAGAATTGGGAACATGCTAAATACAAATGGTATCTTAAG
GAACTCCAGGATGGCATTGGGCAGCGGCAAACTGTTGTCAGAACATTGAATGCAACTGGGGAAGAAATAATTCAG
CAATCCTCAAAAACAGATGCCAGTATTCTACAGGAAAAATTGGGAAGCCTGAATCTGCGGTGGCAGGAGGTCTGC
AAACAGCTGTCAGACAGAAAAAAGAGGCTAGAAGAACAAAAGAATATCTTGTCAGAATTTCAAAGAGATTTAAAT
GAATTTGTTTTATGGTTGGAGGAAGCAGATAACATTGCTAGTATCCCACTTGAACCTGGAAAAGAGCAGCAACTA
AAAGAAAAGCTTGAGCAAGTCAAGTTACTGGTGGAAGAGTTGCCCCTGCGCCAGGGAATTCTCAAACAATTAAAT
GAAACTGGAGGACCCGTGCTTGTAAGTGCTCCCATAAGCCCAGAAGAGCAAGATAAACTTGAAAATAAGCTCAAG
CAGACAAATCTCCAGTGGATAAAGGTTTCCAGAGCTTTACCTGAGAAACAAGGAGAAATTGAAGCTCAAATAAAA
GACCTTGGGCAGCTTGAAAAAAAGCTTGAAGACCTTGAAGAGCAGTTAAATCATCTGCTGCTGTGGTTATCTCCT
ATTAGGAATCAGTTGGAAATTTATAACCAACCAAACCAAGAAGGACCATTTGACGTTAAGGAAACTGAAATAGCA
GTTCAAGCTAAACAACCGGATGTGGAAGAGATTTTGTCTAAAGGGCAGCATTTGTACAAGGAAAAACCAGCCACT
CAGCCAGTGAAGAGGAAGTTAGAAGATCTGAGCTCTGAGTGGAAGGCGGTAAACCGTTTACTTCAAGAGCTGAGG
GCAAAGCAGTGGCAGAGAAAAATAGATGAGACCCTTGAAAGACTCCGGGAACTTCAAGAGGCCACGGATGAGCTG
GACCTCAAGCTGCGCCAAGCTGAGGTGATCAAGGGATCCTGGCAGCCCGTGGGCGATCTCCTCATTGACTCTCTC
CAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAAC
ACCAGATGGAAGCTTCTGCAGGTGGCCGTCGAGGACCGAGTCAGGCAGCTGCATGAAGCCCACAGGGACTTTGGT
CCAGCATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTTTAGCT
GACCTGAATAATGTCAGATTCTCAGCTTATAGGACTGCCATGAAACTCCGAAGACTGCAGAAGGCCCTTTGCTTG
GATCTCTTGAGCCTGTCAGCTGCATGTGATGCCTTGGACCAGCACAACCTCAAGCAAAATGACCAGCCCATGGAT
ATCCTGCAGATTATTAATTGTTTGACCACTATTTATGACCGCCTGGAGCAAGAGCACAACAATTTGGTCAACGTC
CCTCTCTGCGTGGATATGTGTCTGAACTGGCTGCTGAATGTTTATGATACGGGACGAACAGGGAGGATCCGTGTC
CTGTCTTTTAAAACTGGCATCATTTCCCTGTGTAAAGCACATTTGGAAGACAAGTACAGATACCTTTTCAAGCAA
GTGGCAAGTTCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCC
GTCCTGCACAGAGTGGCTGCTGCAGAAACTGCCAAGCATCAGGCCAAATGTAACATCTGCAAAGAGTGTCCAATC
ATTGGATTCAGGTACAGGAGTCTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTACATCAGGAGAAGATGTTCGAGAC
TTTGCCAAGGTACTAAAAAACAAATTTCGAACCAAAAGGTATTTTGCGAAGCATCCCCGAATGGGCTACCTGCCA
GTGCAGACTGTCTTAGAGGGGGACAACATGGAAACTCCCGTTACTCTGATCAACTTCTGGCCAGTAGATTCTGCG
CCTGCCTCGTCCCCTCAGCTTTCACACGATGATACTCATTCACGCATTGAACATTATGCTAGCAGGCTAGCAGAA
ATGGAAAACAGCAATGGATCTTATCTAAATGATAGCATCTCTCCTAATGAGAGCATAGATGATGAACATTTGTTA
ATCCAGCATTACTGCCAAAGTTTGAACCAGGACTCCCCCCTGAGCCAGCCTCGTAGTCCTGCCCAGATCTTGATT
TCCTTAGAGAGTGAGGAAAGAGGGGAGCTAGAGAGAATCCTAGCAGATCTTGAGGAAGAAAACAGGAATCTGCAA
GCAGAATATGACCGTCTAAAGCAGCAGCACGAACATAAAGGCCTGTCCCCACTGCCGTCCCCTCCTGAAATGATG
CCCACCTCTCCCCAGAGTCCC
Nterm-R1_R17-R19_R24 to 1st coiled coil and proline rich
region that follows:
(SEQ ID NO: 129)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATAGATCTGTTGAGAAATGGCGGCGTTTTCATTATGATATAAAGATATTTAATCAGTGGCTAACAGAAGCTGAA
CAGTTTCTCAGAAAGACACAAATTCCTGAGAATTGGGAACATGCTAAATACAAATGGTATCTTAAGGAACTCCAG
GATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTG
TCAGACAGAAAAAAGAGGCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGCAGCAACTAAAAGAAAAG
CTTGAGCAAGTCAAGTTACTGGTGGAAGAGTTGCCCCTGCGCCAGGGAATTCTCAAACAATTAAATGAAACTGGA
GGACCCGTGCTTGTAAGTGCTCCCATAAGCCCAGAAGAGCAAGATAAACTTGAAAATAAGCTCAAGCAGACAAAT
CTCCAGTGGATAAAGGTTTCCAGAGCTTTACCTGAGAAACAAGGAGAAATTGAAGCTCAAATAAAAGACCTTGGG
CAGCTTGAAAAAAAGCTTGAAGACCTTGAAGAGCAGTTAAATCATCTGCTGCTGTGGTTATCTCCTATTAGGAAT
CAGTTGGAAATTTATAACCAACCAAACCAAGAAGGACCATTTGACGTTAAGGAAACTGAAATAGCAGTTCAAGCT
AAACAACCGGATGTGGAAGAGATTTTGTCTAAAGGGCAGCATTTGTACAAGGAAAAACCAGCCACTCAGCCAGTG
AAGAGGAAGTTAGAAGATCTGAGCTCTGAGTGGAAGGCGGTAAACCGTTTACTTCAAGAGCTGAGGGCAAAGCAG
TGGCAGAGAAAAATAGATGAGACCCTTGAAAGACTCCGGGAACTTCAAGAGGCCACGGATGAGCTGGACCTCAAG
CTGCGCCAAGCTGAGGTGATCAAGGGATCCTGGCAGCCCGTGGGCGATCTCCTCATTGACTCTCTCCAAGATCAC
CTCGAGAAAGTCAAGGCACTTCGAGGAGAAATTGCGCCTCTGAAAGAGAACGTGAGCCACGTCAATGACCTTGCT
CGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTTGGTCCAGCATCT
CAGCACTTTCTTTCCACGTCTGTCCAGGGTCCCTGGGAGAGAGCCATCTCGCCAAACAAAGTGCCCTACTATATC
AACCACGAGACTCAAACAACTTGCTGGGACCATCCCAAAATGACAGAGCTCTACCAGTCTTTAGCTGACCTGAAT
AATGTCAGATTCTCAGCTTATAGGACTGCCATGAAACTCCGAAGACTGCAGAAGGCCCTTTGCTTGGATCTCTTG
AGCCTGTCAGCTGCATGTGATGCCTTGGACCAGCACAACCTCAAGCAAAATGACCAGCCCATGGATATCCTGCAG
ATTATTAATTGTTTGACCACTATTTATGACCGCCTGGAGCAAGAGCACAACAATTTGGTCAACGTCCCTCTCTGC
GTGGATATGTGTCTGAACTGGCTGCTGAATGTTTATGATACGGGACGAACAGGGAGGATCCGTGTCCTGTCTTTT
AAAACTGGCATCATTTCCCTGTGTAAAGCACATTTGGAAGACAAGTACAGATACCTTTTCAAGCAAGTGGCAAGT
TCAACAGGATTTTGTGACCAGCGCAGGCTGGGCCTCCTTCTGCATGATTCTATCCAAATTCCAAGACAGTTGGGT
GAAGTTGCATCCTTTGGGGGCAGTAACATTGAGCCAAGTGTCCGGAGCTGCTTCCAATTTGCTAATAATAAGCCA
GAGATCGAAGCGGCCCTCTTCCTAGACTGGATGAGACTGGAACCCCAGTCCATGGTGTGGCTGCCCGTCCTGCAC
AGAGTGGCTGCTGCAGAAACTGCCAAGCATCAGGCCAAATGTAACATCTGCAAAGAGTGTCCAATCATTGGATTC
AGGTACAGGAGTCTAAAGCACTTTAATTATGACATCTGCCAAAGCTGCTTTTTTTCTGGTCGAGTTGCAAAAGGC
CATAAAATGCACTATCCCATGGTGGAATATTGCACTCCGACTACATCAGGAGAAGATGTTCGAGACTTTGCCAAG
GTACTAAAAAACAAATTTCGAACCAAAAGGTATTTTGCGAAGCATCCCCGAATGGGCTACCTGCCAGTGCAGACT
GTCTTAGAGGGGGACAACATGGAAACTCCCGTTACTCTGATCAACTTCTGGCCAGTAGATTCTGCGCCTGCCTCG
TCCCCTCAGCTTTCACACGATGATACTCATTCACGCATTGAACATTATGCTAGCAGGCTAGCAGAAATGGAAAAC
AGCAATGGATCTTATCTAAATGATAGCATCTCTCCTAATGAGAGCATAGATGATGAACATTTGTTAATCCAGCAT
TACTGCCAAAGTTTGAACCAGGACTCCCCCCTGAGCCAGCCTCGTAGTCCTGCCCAGATCTTGATTTCCTTAGAG
AGTGAGGAAAGAGGGGAGCTAGAGAGAATCCTAGCAGATCTTGAGGAAGAAAACAGGAATCTGCAAGCAGAATAT
GACCGTCTAAAGCAGCAGCACGAACATAAAGGCCTGTCCCCACTGCCGTCCCCTCCTGAAATGATGCCCACCTCT
CCCCAGAGTCCC
Nterm-R1_R17-R19_H4 to end of dystrophin:
(SEQ ID NO: 11)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCGATTTGACAGATCTGTTGAGAAATGGCGGCGTTTTCATTATGATATAAAGATATTTAATCAGTGGCTAACA
GAAGCTGAACAGTTTCTCAGAAAGACACAAATTCCTGAGAATTGGGAACATGCTAAATACAAATGGTATCTTAAG
GAACTCCAGGATGGCATTGGGCAGCGGCAAACTGTTGTCAGAACATTGAATGCAACTGGGGAAGAAATAATTCAG
CAATCCTCAAAAACAGATGCCAGTATTCTACAGGAAAAATTGGGAAGCCTGAATCTGCGGTGGCAGGAGGTCTGC
AAACAGCTGTCAGACAGAAAAAAGAGGCTAGAAGAACAAAAGAATATCTTGTCAGAATTTCAAAGAGATTTAAAT
GAATTTGTTTTATGGTTGGAGGAAGCAGATAACATTGCTAGTATCCCACTTGAACCTGGAAAAGAGCAGCAACTA
AAAGAAAAGCTTGAGCAAGTCAAGTTACTGGTGGAAGAGTTGCCCCTGCGCCAGGGAATTCTCAAACAATTAAAT
GAAACTGGAGGACCCGTGCTTGTAAGTGCTCCCATAAGCCCAGAAGAGCAAGATAAACTTGAAAATAAGCTCAAG
CAGACAAATCTCCAGTGGATAAAGGTTTCCAGAGCTTTACCTGAGAAACAAGGAGAAATTGAAGCTCAAATAAAA
GACCTTGGGCAGCTTGAAAAAAAGCTTGAAGACCTTGAAGAGCAGTTAAATCATCTGCTGCTGTGGTTATCTCCT
ATTAGGAATCAGTTGGAAATTTATAACCAACCAAACCAAGAAGGACCATTTGACGTTAAGGAAACTGAAATAGCA
GTTCAAGCTAAACAACCGGATGTGGAAGAGATTTTGTCTAAAGGGCAGCATTTGTACAAGGAAAAACCAGCCACT
CAGCCAGTGAAGAGGAAGTTAGAAGATCTGAGCTCTGAGTGGAAGGCGGTAAACCGTTTACTTCAAGAGCTGAGG
GCAAAGCAGGGTCCAGCATCTCAGCACTTTCTTTCCACGTCTGTCCAGGGTCCCTGGGAGAGAGCCATCTCGCCA
AACAAAGTGCCCTACTATATCAACCACGAGACTCAAACAACTTGCTGGGACCATCCCAAAATGACAGAGCTCTAC
CAGTCTTTAGCTGACCTGAATAATGTCAGATTCTCAGCTTATAGGACTGCCATGAAACTCCGAAGACTGCAGAAG
GCCCTTTGCTTGGATCTCTTGAGCCTGTCAGCTGCATGTGATGCCTTGGACCAGCACAACCTCAAGCAAAATGAC
CAGCCCATGGATATCCTGCAGATTATTAATTGTTTGACCACTATTTATGACCGCCTGGAGCAAGAGCACAACAAT
TTGGTCAACGTCCCTCTCTGCGTGGATATGTGTCTGAACTGGCTGCTGAATGTTTATGATACGGGACGAACAGGG
AGGATCCGTGTCCTGTCTTTTAAAACTGGCATCATTTCCCTGTGTAAAGCACATTTGGAAGACAAGTACAGATAC
CTTTTCAAGCAAGTGGCAAGTTCAACAGGATTTTGTGACCAGCGCAGGCTGGGCCTCCTTCTGCATGATTCTATC
CAAATTCCAAGACAGTTGGGTGAAGTTGCATCCTTTGGGGGCAGTAACATTGAGCCAAGTGTCCGGAGCTGCTTC
CAATTTGCTAATAATAAGCCAGAGATCGAAGCGGCCCTCTTCCTAGACTGGATGAGACTGGAACCCCAGTCCATG
GTGTGGCTGCCCGTCCTGCACAGAGTGGCTGCTGCAGAAACTGCCAAGCATCAGGCCAAATGTAACATCTGCAAA
GAGTGTCCAATCATTGGATTCAGGTACAGGAGTCTAAAGCACTTTAATTATGACATCTGCCAAAGCTGCTTTTTT
TCTGGTCGAGTTGCAAAAGGCCATAAAATGCACTATCCCATGGTGGAATATTGCACTCCGACTACATCAGGAGAA
GATGTTCGAGACTTTGCCAAGGTACTAAAAAACAAATTTCGAACCAAAAGGTATTTTGCGAAGCATCCCCGAATG
GGCTACCTGCCAGTGCAGACTGTCTTAGAGGGGGACAACATGGAAACTCCCGTTACTCTGATCAACTTCTGGCCA
GTAGATTCTGCGCCTGCCTCGTCCCCTCAGCTTTCACACGATGATACTCATTCACGCATTGAACATTATGCTAGC
AGGCTAGCAGAAATGGAAAACAGCAATGGATCTTATCTAAATGATAGCATCTCTCCTAATGAGAGCATAGATGAT
GAACATTTGTTAATCCAGCATTACTGCCAAAGTTTGAACCAGGACTCCCCCCTGAGCCAGCCTCGTAGTCCTGCC
CAGATCTTGATTTCCTTAGAGAGTGAGGAAAGAGGGGAGCTAGAGAGAATCCTAGCAGATCTTGAGGAAGAAAAC
AGGAATCTGCAAGCAGAATATGACCGTCTAAAGCAGCAGCACGAACATAAAGGCCTGTCCCCACTGCCGTCCCCT
CCTGAAATGATGCCCACCTCTCCCCAGAGTCCCCGGGATGCTGAGCTCATTGCTGAGGCCAAGCTACTGCGTCAA
CACAAAGGCCGCCTGGAAGCCAGGATGCAAATCCTGGAAGACCACAATAAACAGCTGGAGTCACAGTTACACAGG
CTAAGGCAGCTGCTGGAGCAACCCCAGGCAGAGGCCAAAGTGAATGGCACAACGGTGTCCTCTCCTTCTACCTCT
CTACAGAGGTCCGACAGCAGTCAGCCTATGCTGCTCCGAGTGGTTGGCAGTCAAACTTCGGACTCCATGGGTGAG
GAAGATCTTCTCAGTCCTCCCCAGGACACAAGCACAGGGTTAGAGGAGGTGATGGAGCAACTCAACAACTCCTTC
CCTAGTTCAAGAGGAAGAAATACCCCTGGAAAGCCAATGAGAGAGGACACAATG
Nterm-R1_R17-R19_H4 to end of dystrophin:
(SEQ ID NO: 130)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGITACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATAGATCTGTTGAGAAATGGCGGCGTTTTCATTATGATATAAAGATATTTAATCAGTGGCTAACAGAAGCTGAA
CAGTTTCTCAGAAAGACACAAATTCCTGAGAATTGGGAACATGCTAAATACAAATGGTATCTTAAGGAACTCCAG
GATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTG
TCAGACAGAAAAAAGAGGCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGCAGCAACTAAAAGAAAAG
CTTGAGCAAGTCAAGTTACTGGTGGAAGAGTTGCCCCTGCGCCAGGGAATTCTCAAACAATTAAATGAAACTGGA
GGACCCGTGCTTGTAAGTGCTCCCATAAGCCCAGAAGAGCAAGATAAACTTGAAAATAAGCTCAAGCAGACAAAT
CTCCAGTGGATAAAGGTTTCCAGAGCTTTACCTGAGAAACAAGGAGAAATTGAAGCTCAAATAAAAGACCTTGGG
CAGCTTGAAAAAAAGCTTGAAGACCTTGAAGAGCAGTTAAATCATCTGCTGCTGTGGTTATCTCCTATTAGGAAT
CAGTTGGAAATTTATAACCAACCAAACCAAGAAGGACCATTIGACGTTAAGGAAACTGAAATAGCAGTTCAAGCT
AAACAACCGGATGTGGAAGAGATTTTGTCTAAAGGGCAGCATTTGTACAAGGAAAAACCAGCCACTCAGCCAGTG
AAGAGGAAGTTAGAAGATCTGAGCTCTGAGTGGAAGGCGGTAAACCGTTTACTTCAAGAGCTGAGGGCAAAGCAG
GGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTA
GCTGACCTGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAACAATTTGGTCAAC
GTCCCTCTCTGCGTGGATATGTGTCTGAACTGGCTGCTGAATGTTTATGATACGGGACGAACAGGGAGGATCCGT
GTCCTGTCTTTTAAAACTGGCATCATTTCCCTGTGTAAAGCACATTTGGAAGACAAGTACAGATACCTTTTCAAG
CAAGTGGCAAGTTCAACAGGATTTTGTGACCAGCGCAGGCTGGGCCTCCTTCTGCATGATTCTATCCAAATTCCA
AGACAGTTGGGTGAAGTTGCATCCTTTGGGGGCAGTAACATTGAGCCAAGTGTCCGGAGCTGCTTCCAATTTGCT
AATAATAAGCCAGAGATCGAAGCGGCCCTCTTCCTAGACTGGATGAGACTGGAACCCCAGTCCATGGTGTGGCTG
CCCGTCCTGCACAGAGTGGCTGCTGCAGAAACTGCCAAGCATCAGGCCAAATGTAACATCTGCAAAGAGTGTCCA
ATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGA
GACTTTGCCAAGGTACTAAAAAACAAATTTCGAACCAAAAGGTATTTTGCGAAGCATCCCCGAATGGGCTACCTG
CCAGTGCAGACTGTCTTAGAGGGGGACAACATGGAAACTCCCGTTACTCTGATCAACTTCTGGCCAGTAGATTCT
GCGCCTGCCTCGTCCCCTCAGCTTTCACACGATGATACTCATTCACGCATTGAACATTATGCTAGCAGGCTAGCA
GAAATGGAAAACAGCAATGGATCTTATCTAAATGATAGCATCTCTCCTAATGAGAGCATAGATGATGAACATTTG
TTAATCCAGCATTACTGCCAAAGTTTGAACCAGGACTCCCCCCTGAGCCAGCCTCGTAGTCCTGCCCAGATCTTG
ATTTCCTTAGAGAGTGAGGAAAGAGGGGAGCTAGAGAGAATCCTAGCAGATCTTGAGGAAGAAAACAGGAATCTG
CAAGCAGAATATGACCGTCTAAAGCAGCAGCACGAACATAAAGGCCTGTCCCCACTGCCGTCCCCTCCTGAAATG
ATGCCCACCTCTCCCCAGAGTCCCCGGGATGCTGAGCTCATTGCTGAGGCCAAGCTACTGCGTCAACACAAAGGC
CGCCTGGAAGCCAGGATGCAAATCCTGGAAGACCACAATAAACAGCTGGAGTCACAGTTACACAGGCTAAGGCAG
CTGCTGGAGCAACCCCAGGCAGAGGCCAAAGTGAATGGCACAACGGTGTCCTCTCCTTCTACCTCTCTACAGAGG
TCCGACAGCAGTCAGCCTATGCTGCTCCGAGTGGTTGGCAGTCAAACTTCGGACTCCATGGGTGAGGAAGATCTT
CTCAGTCCTCCCCAGGACACAAGCACAGGGTTAGAGGAGGTGATGGAGCAACTCAACAACTCCTTCCCTAGTTCA
AGAGGAAGAAATACCCCTGGAAAGCCAATGAGAGAGGACACAATG
Nterm-R1_R17-R19_R24 to end of dystrophin:
(SEQ ID NO: 12)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCGATTTGACAGATCTGTTGAGAAATGGCGGCGTTTTCATTATGATATAAAGATATTTAATCAGTGGCTAACA
GAAGCTGAACAGTTTCTCAGAAAGACACAAATTCCTGAGAATTGGGAACATGCTAAATACAAATGGTATCTTAAG
GAACTCCAGGATGGCATTGGGCAGCGGCAAACTGTTGTCAGAACATTGAATGCAACTGGGGAAGAAATAATTCAG
CAATCCTCAAAAACAGATGCCAGTATTCTACAGGAAAAATTGGGAAGCCTGAATCTGCGGTGGCAGGAGGTCTGC
AAACAGCTGTCAGACAGAAAAAAGAGGCTAGAAGAACAAAAGAATATCTTGTCAGAATTTCAAAGAGATTTAAAT
GAATTTGTTTTATGGTTGGAGGAAGCAGATAACATTGCTAGTATCCCACTTGAACCTGGAAAAGAGCAGCAACTA
AAAGAAAAGCTTGAGCAAGTCAAGTTACTGGTGGAAGAGTTGCCCCTGCGCCAGGGAATTCTCAAACAATTAAAT
GAAACTGGAGGACCCGTGCTTGTAAGTGCTCCCATAAGCCCAGAAGAGCAAGATAAACTTGAAAATAAGCTCAAG
CAGACAAATCTCCAGTGGATAAAGGTTTCCAGAGCTTTACCTGAGAAACAAGGAGAAATTGAAGCTCAAATAAAA
GACCTTGGGCAGCTTGAAAAAAAGCTTGAAGACCTTGAAGAGCAGTTAAATCATCTGCTGCTGTGGTTATCTCCT
ATTAGGAATCAGTTGGAAATTTATAACCAACCAAACCAAGAAGGACCATTTGACGTTAAGGAAACTGAAATAGCA
GTTCAAGCTAAACAACCGGATGTGGAAGAGATTTTGTCTAAAGGGCAGCATTTGTACAAGGAAAAACCAGCCACT
CAGCCAGTGAAGAGGAAGTTAGAAGATCTGAGCTCTGAGTGGAAGGCGGTAAACCGTTTACTTCAAGAGCTGAGG
GCAAAGCAGTGGCAGAGAAAAATAGATGAGACCCTTGAAAGACTCCGGGAACTTCAAGAGGCCACGGATGAGCTG
GACCTCAAGCTGCGCCAAGCTGAGGTGATCAAGGGATCCTGGCAGCCCGTGGGCGATCTCCTCATTGACTCTCTC
CAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAAC
ACCAGATGGAAGCTTCTGCAGGTGGCCGTCGAGGACCGAGTCAGGCAGCTGCATGAAGCCCACAGGGACTTTGGT
CCAGCATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTTTAGCT
GACCTGAATAATGTCAGATTCTCAGCTTATAGGACTGCCATGAAACTCCGAAGACTGCAGAAGGCCCTTTGCTTG
GATCTCTTGAGCCTGTCAGCTGCATGTGATGCCTTGGACCAGCACAACCTCAAGCAAAATGACCAGCCCATGGAT
ATCCTGCAGATTATTAATTGTTTGACCACTATTTATGACCGCCTGGAGCAAGAGCACAACAATTTGGTCAACGTC
CCTCTCTGCGTGGATATGTGTCTGAACTGGCTGCTGAATGTTTATGATACGGGACGAACAGGGAGGATCCGTGTC
CTGTCTTTTAAAACTGGCATCATTTCCCTGTGTAAAGCACATTTGGAAGACAAGTACAGATACCTTTTCAAGCAA
GTGGCAAGITCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCC
GTCCTGCACAGAGTGGCTGCTGCAGAAACTGCCAAGCATCAGGCCAAATGTAACATCTGCAAAGAGTGTCCAATC
ATTGGATTCAGGTACAGGAGTCTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTACATCAGGAGAAGATGTTCGAGAC
TTTGCCAAGGTACTAAAAAACAAATTTCGAACCAAAAGGTATTTTGCGAAGCATCCCCGAATGGGCTACCTGCCA
GTGCAGACTGTCTTAGAGGGGGACAACATGGAAACTCCCGTTACTCTGATCAACTTCTGGCCAGTAGATTCTGCG
CCTGCCTCGTCCCCTCAGCTTTCACACGATGATACTCATTCACGCATTGAACATTATGCTAGCAGGCTAGCAGAA
ATGGAAAACAGCAATGGATCTTATCTAAATGATAGCATCTCTCCTAATGAGAGCATAGATGATGAACATTTGTTA
ATCCAGCATTACTGCCAAAGTTTGAACCAGGACTCCCCCCTGAGCCAGCCTCGTAGTCCTGCCCAGATCTTGATT
TCCTTAGAGAGTGAGGAAAGAGGGGAGCTAGAGAGAATCCTAGCAGATCTTGAGGAAGAAAACAGGAATCTGCAA
GCAGAATATGACCGTCTAAAGCAGCAGCACGAACATAAAGGCCTGTCCCCACTGCCGTCCCCTCCTGAAATGATG
CCCACCTCTCCCCAGAGTCCCCGGGATGCTGAGCTCATTGCTGAGGCCAAGCTACTGCGTCAACACAAAGGCCGC
CTGGAAGCCAGGATGCAAATCCTGGAAGACCACAATAAACAGCTGGAGTCACAGTTACACAGGCTAAGGCAGCTG
CTGGAGCAACCCCAGGCAGAGGCCAAAGTGAATGGCACAACGGTGTCCTCTCCTTCTACCTCTCTACAGAGGTCC
GACAGCAGTCAGCCTATGCTGCTCCGAGTGGTTGGCAGTCAAACTTCGGACTCCATGGGTGAGGAAGATCTTCTC
AGTCCTCCCCAGGACACAAGCACAGGGTTAGAGGAGGTGATGGAGCAACTCAACAACTCCTTCCCTAGTTCAAGA
GGAAGAAATACCCCTGGAAAGCCAATGAGAGAGGACACAATG
Nterm-R1_R17-R19_R24 to end of dystrophin:
(SEQ ID NO: 131)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATAGATCTGTTGAGAAATGGCGGCGTTTTCATTATGATATAAAGATATTTAATCAGTGGCTAACAGAAGCTGAA
CAGTTTCTCAGAAAGACACAAATTCCTGAGAATTGGGAACATGCTAAATACAAATGGTATCTTAAGGAACTCCAG
GATGGCATTGGGCAGCGGCAAACTGTTGTCAGAACATTGAATGCAACTGGGGAAGAAATAATTCAGCAATCCTCA
AAAACAGATGCCAGTATTCTACAGGAAAAATTGGGAAGCCTGAATCTGCGGTGGCAGGAGGTCTGCAAACAGCTG
TCAGACAGAAAAAAGAGGCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGCAGCAACTAAAAGAAAAG
CTTGAGCAAGTCAAGTTACTGGTGGAAGAGTTGCCCCTGCGCCAGGGAATTCTCAAACAATTAAATGAAACTGGA
GGACCCGTGCTTGTAAGTGCTCCCATAAGCCCAGAAGAGCAAGATAAACTTGAAAATAAGCTCAAGCAGACAAAT
CTCCAGTGGATAAAGGTTTCCAGAGCTTTACCTGAGAAACAAGGAGAAATTGAAGCTCAAATAAAAGACCTTGGG
CAGCTTGAAAAAAAGCTTGAAGACCTTGAAGAGCAGTTAAATCATCTGCTGCTGTGGTTATCTCCTATTAGGAAT
CAGTTGGAAATTTATAACCAACCAAACCAAGAAGGACCATTTGACGTTAAGGAAACTGAAATAGCAGTTCAAGCT
AAACAACCGGATGTGGAAGAGATTTTGTCTAAAGGGCAGCATTTGTACAAGGAAAAACCAGCCACTCAGCCAGTG
AAGAGGAAGTTAGAAGATCTGAGCTCTGAGTGGAAGGCGGTAAACCGTTTACTTCAAGAGCTGAGGGCAAAGCAG
TGGCAGAGAAAAATAGATGAGACCCTTGAAAGACTCCGGGAACTTCAAGAGGCCACGGATGAGCTGGACCTCAAG
CTGCGCCAAGCTGAGGTGATCAAGGGATCCTGGCAGCCCGTGGGCGATCTCCTCATTGACTCTCTCCAAGATCAC
CTCGAGAAAGTCAAGGCACTTCGAGGAGAAATTGCGCCTCTGAAAGAGAACGTGAGCCACGTCAATGACCTTGCT
CGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTTGGTCCAGCATCT
CAGCACTTTCTTTCCACGTCTGTCCAGGGTCCCTGGGAGAGAGCCATCTCGCCAAACAAAGTGCCCTACTATATC
AACCACGAGACTCAAACAACTTGCTGGGACCATCCCAAAATGACAGAGCTCTACCAGTCTTTAGCTGACCTGAAT
AATGTCAGATTCTCAGCTTATAGGACTGCCATGAAACTCCGAAGACTGCAGAAGGCCCTTTGCTTGGATCTCTTG
AGCCTGTCAGCTGCATGTGATGCCTTGGACCAGCACAACCTCAAGCAAAATGACCAGCCCATGGATATCCTGCAG
ATTATTAATTGTTTGACCACTATTTATGACCGCCTGGAGCAAGAGCACAACAATTTGGTCAACGTCCCTCTCTGC
GTGGATATGTGTCTGAACTGGCTGCTGAATGTTTATGATACGGGACGAACAGGGAGGATCCGTGTCCTGTCTTTT
AAAACTGGCATCATTTCCCTGTGTAAAGCACATTTGGAAGACAAGTACAGATACCTTTTCAAGCAAGTGGCAAGT
TCAACAGGATTTTGTGACCAGCGCAGGCTGGGCCTCCTTCTGCATGATTCTATCCAAATTCCAAGACAGTTGGGT
GAAGTTGCATCCTTTGGGGGCAGTAACATTGAGCCAAGTGTCCGGAGCTGCTTCCAATTTGCTAATAATAAGCCA
GAGATCGAAGCGGCCCTCTTCCTAGACTGGATGAGACTGGAACCCCAGTCCATGGTGTGGCTGCCCGTCCTGCAC
AGAGTGGCTGCTGCAGAAACTGCCAAGCATCAGGCCAAATGTAACATCTGCAAAGAGTGTCCAATCATTGGATTC
AGGTACAGGAGTCTAAAGCACTTTAATTATGACATCTGCCAAAGCTGCTTTTTTTCTGGTCGAGTTGCAAAAGGC
CATAAAATGCACTATCCCATGGTGGAATATTGCACTCCGACTACATCAGGAGAAGATGTTCGAGACTTTGCCAAG
GTACTAAAAAACAAATTTCGAACCAAAAGGTATTTTGCGAAGCATCCCCGAATGGGCTACCTGCCAGTGCAGACT
GTCTTAGAGGGGGACAACATGGAAACTCCCGTTACTCTGATCAACTTCTGGCCAGTAGATTCTGCGCCTGCCTCG
TCCCCTCAGCTTTCACACGATGATACTCATTCACGCATTGAACATTATGCTAGCAGGCTAGCAGAAATGGAAAAC
AGCAATGGATCTTATCTAAATGATAGCATCTCTCCTAATGAGAGCATAGATGATGAACATTTGTTAATCCAGCAT
TACTGCCAAAGTTTGAACCAGGACTCCCCCCTGAGCCAGCCTCGTAGTCCTGCCCAGATCTTGATTTCCTTAGAG
AGTGAGGAAAGAGGGGAGCTAGAGAGAATCCTAGCAGATCTTGAGGAAGAAAACAGGAATCTGCAAGCAGAATAT
GACCGTCTAAAGCAGCAGCACGAACATAAAGGCCTGTCCCCACTGCCGTCCCCTCCTGAAATGATGCCCACCTCT
CCCCAGAGTCCCCGGGATGCTGAGCTCATTGCTGAGGCCAAGCTACTGCGTCAACACAAAGGCCGCCTGGAAGCC
AGGATGCAAATCCTGGAAGACCACAATAAACAGCTGGAGTCACAGTTACACAGGCTAAGGCAGCTGCTGGAGCAA
CCCCAGGCAGAGGCCAAAGTGAATGGCACAACGGTGTCCTCTCCTTCTACCTCTCTACAGAGGTCCGACAGCAGT
CAGCCTATGCTGCTCCGAGTGGTTGGCAGTCAAACTTCGGACTCCATGGGTGAGGAAGATCTTCTCAGTCCTCCC
CAGGACACAAGCACAGGGTTAGAGGAGGTGATGGAGCAACTCAACAACTCCTTCCCTAGTTCAAGAGGAAGAAAT
ACCCCTGGAAAGCCAATGAGAGAGGACACAATG

### Cardiac Muscle Micro-Dystrophins

The present disclosure provides micro-dystrophin genes and proteins optimized for cardiac muscle. The inventors have found that certain combinations of domains from full-length dystrophin are beneficial for cardiac muscle. However, these same micro-dystrophins are also beneficial for skeletal muscle.

In some embodiments, a micro-dystrophin protein optimized for cardiac muscle does not comprise spectrin-like repeat 2 and/or spectrin-like repeat 3 of dystrophin. In some embodiments, a micro-dystrophin protein does not comprise spectrin-like repeat 2. In some embodiments, a micro-dystrophin protein does not comprise spectrin-like repeat 3. In some embodiments, a micro-dystrophin protein does not comprise spectrin-like repeat 2 and spectrin-like repeat 3. The inventors have found that including spectrin-like repeat 1 but not spectrin-like repeat 2 and/or spectrin-like repeat 3 is beneficial to the heart as such micro-dystrophin proteins outcompete utrophin for its membrane binding in the heart to a lesser extent, thus mitigating the harmful effects of displacing utrophin. Harmful effects of displacing utrophin include cardiomyopathy and heart failure.

In some embodiments, a micro-dystrophin protein optimized for cardiac muscle comprises an amino-terminal actin-binding domain, hinge domain 1, spectrin-like repeat 1, spectrin-like repeat 17, spectrin-like repeat 18, and spectrin-like repeat 19 of dystrophin. In some embodiments, the micro-dystrophin protein does not contain spectrin-like repeat 2 and/or spectrin-like repeat 3 of dystrophin. In some embodiments, the micro-dystrophin protein does not comprise spectrin-like repeat 2. In some embodiments, the micro-dystrophin protein does not comprise spectrin-like repeat 3. In some embodiments, the micro-dystrophin protein does not comprise spectrin-like repeat 2 and spectrin-like repeat 3.

In some embodiments, a micro-dystrophin protein optimized for cardiac muscle comprises one or more of: spectrin-like repeat 24, hinge domain 4, and dystroglycan binding site of dystrophin. In some embodiments, a micro-dystrophin protein optimized for cardiac muscle comprises spectrin-like repeat 24 and hinge domain 4. In some embodiments, a micro-dystrophin protein optimized for cardiac muscle comprises spectrin-like repeat 24 and the dystroglycan binding site of dystrophin. In some embodiments, a micro-dystrophin protein optimized for cardiac muscle comprises hinge domain 4 and the dystroglycan binding site of dystrophin.

In some embodiments, a micro-dystrophin protein optimized for cardiac muscle comprises one or more syntrophin binding domains of dystrophin.

In some embodiments, a micro-dystrophin protein optimized for cardiac muscle comprises one or more coiled coil domains of dystrophin.

In some embodiments, a micro-dystrophin protein optimized for cardiac muscle comprises spectrin-like repeat 1 directly coupled to spectrin-like repeat 17.

In some embodiments, a micro-dystrophin protein optimized for cardiac muscle comprises spectrin-like repeat 19 directly coupled to spectrin-like repeat 24. In some embodiments, a micro-dystrophin protein optimized for cardiac muscle comprises spectrin-like repeat 19 directly coupled to hinge domain 4.

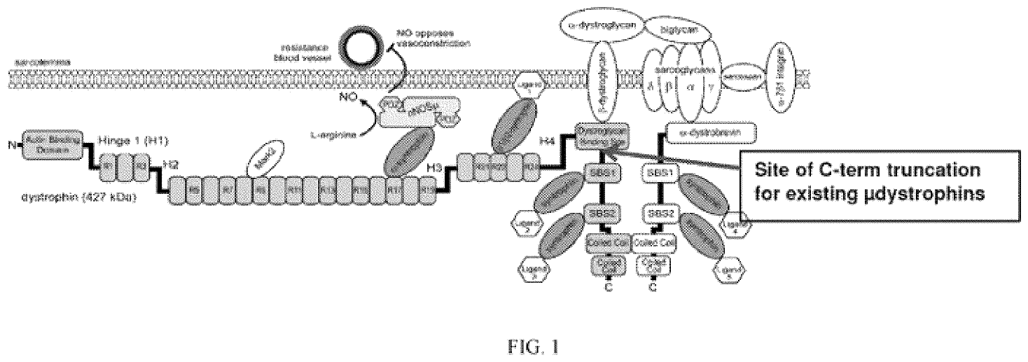

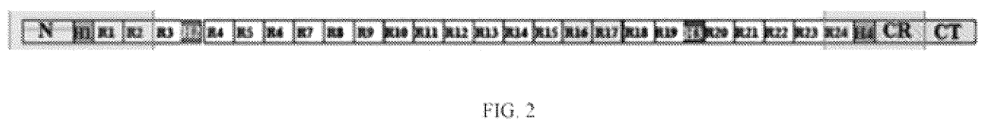

In some embodiments, a micro-dystrophin protein optimized for cardiac muscle comprises the region of dystrophin that is C-terminal to the cysteine-rich portion of the C-terminal region (FIGS. 1 and 2). In some embodiments, a micro-dystrophin protein optimized for cardiac muscle comprises the region of dystrophin that is C-terminal to the dystroglycan binding site of dystrophin.

In some embodiments, a micro-dystrophin protein optimized for cardiac muscle comprises the region of dystrophin from spectrin-like repeat 24 to the C-terminus. In some embodiments, a micro-dystrophin protein optimized for cardiac muscle comprises the region of dystrophin from spectrin-like repeat 24 to the end of the proline rich region following the first coiled coil domain.

In some embodiments, a micro-dystrophin protein optimized for cardiac muscle comprises the region of dystrophin from hinge domain 4 to the C-terminus. In some embodiments, a micro-dystrophin protein optimized for cardiac muscle comprises the region of dystrophin from hinge domain 4 to the end of the proline rich region following the first coiled coil domain. In some embodiments, a micro-dystrophin protein optimized for cardiac muscle comprises the region of dystrophin from hinge domain 4 to the end of the second syntrophin binding domain (or “syntrophin region” or “syntrophin binding site”).

Domains of the dystrophin protein are known in the art. Exemplary sequences of domains of the dystrophin protein or combinations thereof are provided in Examples 1 and 4. In some embodiments, a protein of the disclosure comprises an amino-terminal actin-binding domain and hinge domain 1 region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 40. In some embodiments, a protein of the disclosure comprises an amino-terminal actin-binding domain and hinge domain 1 region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 203. In some embodiments, a protein of the disclosure comprises a spectrin-like repeat 1 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 41. In some embodiments, a protein of the disclosure comprises a spectrin-like repeat 2 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 42. In some embodiments, a protein of the disclosure comprises a spectrin-like repeat 3 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 43. In some embodiments, a protein of the disclosure comprises a spectrin-like repeat 16 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 44. In some embodiments, a protein of the disclosure comprises a spectrin-like repeat 16 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 44 without the linker sequence. In some embodiments, a protein of the disclosure comprises a spectrin-like repeat 16 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 197. In some embodiments, a protein of the disclosure comprises a spectrin-like repeat 17 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 45. In some embodiments, a protein of the disclosure comprises a spectrin-like repeat 17 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 198. In some embodiments, a protein of the disclosure comprises spectrin-like repeats 17-19 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 46. In some embodiments, a protein of the disclosure comprises spectrin-like repeats 17-19 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 186. In some embodiments, a protein of the disclosure comprises spectrin-like repeats 18-19 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 199. In some embodiments, a protein of the disclosure comprises a hinge domain 3 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 204. In some embodiments, a protein of the disclosure comprises a spectrin-like repeat 21 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 205. In some embodiments, a protein of the disclosure comprises a spectrin-like repeat 22 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 206. In some embodiments, a protein of the disclosure comprises a spectrin-like repeat 22 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 206 without the linker sequence. In some embodiments, a protein of the disclosure comprises a spectrin-like repeat 23 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 47. In some embodiments, a protein of the disclosure comprises a spectrin-like repeat 23 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 187. In some embodiments, a protein of the disclosure comprises a spectrin-like repeat 23 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 200. In some embodiments, a protein of the disclosure comprises a spectrin-like repeat 24 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 48. In some embodiments, a protein of the disclosure comprises a spectrin-like repeat 24 comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 201. In some embodiments, a protein of the disclosure comprises a hinge domain 4 and dystroglycan binding site (also referred to herein as “trCterm”) comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 49. In some embodiments, a protein of the disclosure comprises a hinge domain 4 and dystroglycan binding site (also referred to herein as “trCterm”) comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 202. In some embodiments, a protein of the disclosure comprises a syntrophin binding region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 50. In some embodiments, a protein of the disclosure comprises a 1st coiled coil region and proline rich region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 51. In some embodiments, a protein of the disclosure comprises a 1st coiled coil region comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 51 without the proline rich region. In some embodiments, a protein of the disclosure comprises a 2nd coiled coil region to end of dystrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 52.

Domains of the utrophin protein are known in the art. Exemplary sequences of domains of the utrophin protein or combinations thereof are provided in Example 4. In some embodiments, a protein of the disclosure comprises an amino-terminal domain of utrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 191. In some embodiments, a protein of the disclosure comprises a repeat 1 domain of utrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 192. In some embodiments, a protein of the disclosure comprises a repeat 2 domain of utrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 193. In some embodiments, a protein of the disclosure comprises a repeat 3 domain of utrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 194. In some embodiments, a protein of the disclosure comprises a hinge domain 2 of utrophin comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 195.

In some embodiments, a protein of the disclosure comprises an amino acid sequence that has up to 10 (e.g., 1, 2, 3, 4, 5, 6, 7, 8, 9, or 10) amino acid additions or deletions relative to a sequence provided herein (e.g., any one of SEQ ID NOs. 40-52, 186, 187, 191-195, 197-206).

A nucleotide sequence encoding a micro-dystrophin protein optimized for cardiac muscle is further understood to include nucleotide sequences that are variants of any one of SEQ ID Nos. 7-12, 85-92, and 126-131. Variant nucleotide sequences include sequences that differ by one or more (e.g., 1, 2, 3, 4, 5, 6, 7, 8, 9, 10, 11, 12, 13, 14, 15, 16, 17, 18, 19, 20, or more) nucleotide substitutions, additions or deletions, such as allelic variants, and will, therefore, include coding sequences that differ from the nucleotide sequence of the coding sequence designated in any one of SEQ ID Nos. 7-12, 85-92, and 126-131.

In some embodiments, a micro-dystrophin protein optimized for cardiac muscle is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 7-12, 85-92, and 126-131.

In some embodiments, a micro-dystrophin protein optimized for cardiac muscle is encoded by a nucleic acid consisting essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 7-12, 85-92, and 126-131.

In some embodiments, a micro-dystrophin protein optimized for cardiac muscle is encoded by a nucleic acid consisting of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 7-12, 85-92, and 126-131.

In some embodiments, it is desirable to avoid overexpression of micro-dystrophin proteins in the heart. Thus, in some embodiments, a nucleotide sequence encoding a micro-dystrophin protein optimized for cardiac muscle may be codon optimized for lower protein expression. In some embodiments, one or more codons (e.g., 1, 2, 3, 4, 5, 6, 7, 8, 9, 10, 11, 12, 13, 14, 15, 16, 17, 18, 19, 20, or more) are substituted for a rare codon.

Non-limiting examples of human micro-dystrophin proteins optimized for cardiac muscle are described below.

Nterm-R1_R17-R19_R24 + H4 + trCterm:
(SEQ ID NO: 1)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRFDRSVEKWRRFHYDIKIFNQWLTEAEQFLRKTQIPENWEHAKYKWYLKELQDGIGQRQTVVRILNATGEEIIQ
QSSKTDASILQEKLGSLNLRWQEVCKQLSDRKKRLEEQKNILSEFQRDLNEFVLWLEEADNIASIPLEPGKEQQL
KEKLEQVKLLVEELPLRQGILKQLNETGGPVLVSAPISPEEQDKLENKLKQTNLQWIKVSRALPEKQGEIEAQIK
DLGQLEKKLEDLEEQLNHLLLWLSPIRNQLEIYNQPNQEGPFDVKETEIAVQAKQPDVEEILSKGQHLYKEKPAT
QPVKRKLEDLSSEWKAVNRLLQELRAKQWQRKIDETLERLQELQEATDELDLKLRQAEVIKGSWQPVGDLLIDSL
QDHLEKVKALRGEIAPLKENVSHVNDLARQLTTLGIQLSPYNLSTLEDLNTRWKLLQVAVEDRVRQLHEAHRDFG
PASQHFLSTSVQGPWERAISPNKVPYYINHETQTTCWDHPKMTELYQSLADLNNVRFSAYRTAMKLRRLQKALCL
DLLSLSAACDALDQHNLKQNDQPMDILQIINCLTTIYDRLEQEHNNLVNVPLCVDMCLNWLLNVYDTGRTGRIRV
LSFKTGIISLCKAHLEDKYRYLFKQVASSTGFCDQRRLGLLLHDSIQIPRQLGEVASFGGSNIEPSVRSCFQFAN
NKPEIEAALFLDWMRLEPQSMVWLPVLHRVAAAETAKHQAKCNICKECPIIGFRYRSLKHFNYDICQSCFFSGRV
AKGHKMHYPMVEYCTPTTSGEDVRDFAKVLKNKFRTKRYFAKHPRMGYLPVQTVLEGDNMETP
Nterm-R1_R17-R19_R24 + H4 + trCterm:
(SEQ ID NO: 120)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLILGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRSVEKWRRFHYDIKIFNQWLTEAEQFLRKTQIPENWEHAKYKWYLKELQDGIGQRQTVVRTLNATGEEIIQQSS
KTDASILQEKLGSLNLRWQEVCKQLSDRKKRLEEQKNILSEFQRDLNEFVLWLEEADNIASIPLEPGKEQQLKEK
LEQVKLLVEELPLRQGILKQLNETGGPVLVSAPISPEEQDKLENKLKQTNLQWIKVSRALPEKQGEIEAQIKDLG
QLEKKLEDLEEQLNHLLLWLSPIRNQLEIYNQPNQEGPFDVKETEIAVQAKQPDVEEILSKGQHLYKEKPATQPV
KRKLEDLSSEWKAVNRLLQELRAKQWQRKIDETLERLQELQEATDELDLKLRQAEVIKGSWQPVGDLLIDSLQDH
LEKVKALRGEIAPLKENVSHVNDLARQLTTLGIQLSPYNLSTLEDLNTRWKLLQVAVEDRVRQLHEAHRDFGPAS
QHFLSTSVQGPWERAISPNKVPYYINHETQTTCWDHPKMTELYQSLADLNNVRFSAYRTAMKLRRLQKALCLDLL
SLSAACDALDQHNLKQNDQPMDILQIINCLTTIYDRLEQEHNNLVNVPLCVDMCLNWLLNVYDTGRTGRIRVLSF
KTGIISLCKAHLEDKYRYLFKQVASSTGFCDQRRLGLLLHDSIQIPRQLGEVASFGGSNIEPSVRSCFQFANNKP
EIEAALFLDWMRLEPQSMVWLPVLHRVAAAETAKHQAKCNICKECPIIGFRYRSLKHFNYDICQSCFFSGRVAKG
HKMHYPMVEYCTPTTSGEDVRDFAKVLKNKFRTKRYFAKHPRMGYLPVQTVLEGDNMETP
Nterm-R1_R17-R19_H4 to 1st coiled coil and proline rich region that follows:
(SEQ ID NO: 2)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRFDRSVEKWRRFHYDIKIFNQWLTEAEQFLRKTQIPENWEHAKYKWYLKELQDGIGQRQTVVRTLNATGEEIIQ
QSSKTDASILQEKLGSLNLRWQEVCKQLSDRKKRLEEQKNILSEFQRDLNEFVLWLEEADNIASIPLEPGKEQQL
KEKLEQVKLLVEELPLRQGILKQLNETGGPVLVSAPISPEEQDKLENKLKQTNLQWIKVSRALPEKQGEIEAQIK
DLGQLEKKLEDLEEQLNHLLLWLSPIRNQLEIYNQPNQEGPFDVKETEIAVQAKQPDVEEILSKGQHLYKEKPAT
QPVKRKLEDLSSEWKAVNRLLQELRAKQGPASQHFLSTSVQGPWERAISPNKVPYYINHETQTTCWDHPKMTELY
QSLADLNNVRFSAYRTAMKLRRLQKALCLDLLSLSAACDALDQHNLKQNDQPMDILQIINCLTTIYDRLEQEHNN
LVNVPLCVDMCLNWLLNVYDTGRTGRIRVLSFKTGIISLCKAHLEDKYRYLFKQVASSTGFCDQRRLGLLLHDSI
QIPRQLGEVASFGGSNIEPSVRSCFQFANNKPEIEAALFLDWMRLEPQSMVWLPVLHRVAAAETAKHQAKCNICK
ECPIIGFRYRSLKHFNYDICQSCFFSGRVAKGHKMHYPMVEYCTPTTSGEDVRDFAKVLKNKFRTKRYFAKHPRM
GYLPVQTVLEGDNMETPVTLINFWPVDSAPASSPQLSHDDTHSRIEHYASRLAEMENSNGSYLNDSISPNESIDD
EHLLIQHYCQSLNQDSPLSQPRSPAQILISLESEERGELERILADLEEENRNLQAEYDRLKQQHEHKGLSPLPSP
PEMMPTSPQSP
Nterm-R1_R17-R19_H4 to 1st coiled coil and proline rich region that follows:
(SEQ ID NO: 121)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRSVEKWRRFHYDIKIFNQWLTEAEQFLRKTQIPENWEHAKYKWYLKELQDGIGQRQTVVRILNATGEEIIQQSS
KTDASILQEKLGSLNLRWQEVCKQLSDRKKRLEEQKNILSEFQRDLNEFVLWLEEADNIASIPLEPGKEQQLKEK
LEQVKLLVEELPLRQGILKQLNETGGPVLVSAPISPEEQDKLENKLKQTNLQWIKVSRALPEKQGEIEAQIKDLG
QLEKKLEDLEEQLNHLLLWLSPIRNQLEIYNQPNQEGPFDVKETEIAVQAKQPDVEEILSKGQHLYKEKPATQPV
KRKLEDLSSEWKAVNRLLQELRAKQGPASQHFLSTSVQGPWERAISPNKVPYYINHETQTTCWDHPKMTELYQSL
ADLNNVRFSAYRTAMKLRRLQKALCLDLLSLSAACDALDQHNLKQNDQPMDILQIINCLTTIYDRLEQEHNNLVN
VPLCVDMCLNWLLNVYDTGRTGRIRVLSFKTGIISLCKAHLEDKYRYLFKQVASSTGFCDQRRLGLLLHDSIQIP
RQLGEVASFGGSNIEPSVRSCFQFANNKPEIEAALFLDWMRLEPQSMVWLPVLHRVAAAETAKHQAKCNICKECP
IIGFRYRSLKHFNYDICQSCFFSGRVAKGHKMHYPMVEYCTPTTSGEDVRDFAKVLKNKFRTKRYFAKHPRMGYL
PVQTVLEGDNMETPVTLINFWPVDSAPASSPQLSHDDTHSRIEHYASRLAEMENSNGSYLNDSISPNESIDDEHL
LIQHYCQSLNQDSPLSQPRSPAQILISLESEERGELERILADLEEENRNLQAEYDRLKQQHEHKGLSPLPSPPEM
MPTSPQSP
Nterm-R1_R17-R19_H4 to end of syntrophin region:
(SEQ ID NO: 3)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRFDRSVEKWRRFHYDIKIFNQWLTEAEQFLRKTQIPENWEHAKYKWYLKELQDGIGQRQTVVRTLNATGEEIIQ
QSSKTDASILQEKLGSLNLRWQEVCKQLSDRKKRLEEQKNILSEFQRDLNEFVLWLEEADNIASIPLEPGKEQQL
KEKLEQVKLLVEELPLRQGILKQLNETGGPVLVSAPISPEEQDKLENKLKQTNLQWIKVSRALPEKQGEIEAQIK
DLGQLEKKLEDLEEQLNHLLLWLSPIRNQLEIYNQPNQEGPFDVKETEIAVQAKQPDVEEILSKGQHLYKEKPAT
QPVKRKLEDLSSEWKAVNRLLQELRAKQGPASQHFLSTSVQGPWERAISPNKVPYYINHETQTTCWDHPKMTELY
QSLADLNNVRFSAYRTAMKLRRLQKALCLDLLSLSAACDALDQHNLKQNDQPMDILQIINCLTTIYDRLEQEHNN
LVNVPLCVDMCLNWLLNVYDTGRTGRIRVLSFKTGIISLCKAHLEDKYRYLFKQVASSTGFCDQRRLGLLLHDSI
QIPRQLGEVASFGGSNIEPSVRSCFQFANNKPEIEAALFLDWMRLEPQSMVWLPVLHRVAAAETAKHQAKCNICK
ECPIIGFRYRSLKHFNYDICQSCFFSGRVAKGHKMHYPMVEYCTPTTSGEDVRDFAKVLKNKFRTKRYFAKHPRM
GYLPVQTVLEGDNMETPVTLINFWPVDSAPASSPQLSHDDTHSRIEHYASRLAEMENSNGSYLNDSISPNESIDD
EHLLIQHYCQSLNQDSPLSQPRS
Nterm-R1_R17-R19_H4 to end of syntrophin region:
(SEQ ID NO: 122)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRSVEKWRRFHYDIKIFNQWLTEAEQFLRKTQIPENWEHAKYKWYLKELQDGIGQRQTVVRTLNATGEEIIQQSS
KTDASILQEKLGSLNLRWQEVCKQLSDRKKRLEEQKNILSEFQRDLNEFVLWLEEADNIASIPLEPGKEQQLKEK
LEQVKLLVEELPLRQGILKQLNETGGPVLVSAPISPEEQDKLENKLKQTNLQWIKVSRALPEKQGEIEAQIKDLG
QLEKKLEDLEEQLNHLLLWLSPIRNQLEIYNQPNQEGPFDVKETEIAVQAKQPDVEEILSKGQHLYKEKPATQPV
KRKLEDLSSEWKAVNRLLQELRAKQGPASQHFLSTSVQGPWERAISPNKVPYYINHETQTTCWDHPKMTELYQSL
ADLNNVRFSAYRTAMKLRRLQKALCLDLLSLSAACDALDQHNLKQNDQPMDILQIINCLTTIYDRLEQEHNNLVN
VPLCVDMCLNWLLNVYDTGRTGRIRVLSFKTGIISLCKAHLEDKYRYLFKQVASSTGFCDQRRLGLLLHDSIQIP
RQLGEVASFGGSNIEPSVRSCFQFANNKPEIEAALFLDWMRLEPQSMVWLPVLHRVAAAETAKHQAKCNICKECP
IIGFRYRSLKHFNYDICQSCFFSGRVAKGHKMHYPMVEYCTPTTSGEDVRDFAKVLKNKFRTKRYFAKHPRMGYL
PVQTVLEGDNMETPVTLINFWPVDSAPASSPQLSHDDTHSRIEHYASRLAEMENSNGSYLNDSISPNESIDDEHL
LIQHYCQSLNQDSPLSQPRS
N-terminus through R1_R17-R19_R24 to 1st coiled coil and proline rich region that follows:
(SEQ ID NO: 4)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRFDRSVEKWRRFHYDIKIFNQWLTEAEQFLRKTQIPENWEHAKYKWYLKELQDGIGQRQTVVRTLNATGEEIIQ
QSSKTDASILQEKLGSLNLRWQEVCKQLSDRKKRLEEQKNILSEFQRDLNEFVLWLEEADNIASIPLEPGKEQQL
KEKLEQVKLLVEELPLRQGILKQLNETGGPVLVSAPISPEEQDKLENKLKQTNLQWIKVSRALPEKQGEIEAQIK
DLGQLEKKLEDLEEQLNHLLLWLSPIRNQLEIYNQPNQEGPFDVKETEIAVQAKQPDVEEILSKGQHLYKEKPAT
QPVKRKLEDLSSEWKAVNRLLQELRAKQWQRKIDETLERLQELQEATDELDLKLRQAEVIKGSWQPVGDLLIDSL
QDHLEKVKALRGEIAPLKENVSHVNDLARQLTTLGIQLSPYNLSTLEDLNTRWKLLQVAVEDRVRQLHEAHRDFG
PASQHFLSTSVQGPWERAISPNKVPYYINHETQTTCWDHPKMTELYQSLADLNNVRFSAYRTAMKLRRLQKALCL
DLLSLSAACDALDQHNLKQNDQPMDILQIINCLTTIYDRLEQEHNNLVNVPLCVDMCLNWLLNVYDTGRTGRIRV
LSFKTGIISLCKAHLEDKYRYLFKQVASSTGFCDQRRLGLLLHDSIQIPRQLGEVASFGGSNIEPSVRSCFQFAN
NKPEIEAALFLDWMRLEPQSMVWLPVLHRVAAAETAKHQAKCNICKECPIIGFRYRSLKHFNYDICQSCFFSGRV
AKGHKMHYPMVEYCTPTTSGEDVRDFAKVLKNKFRTKRYFAKHPRMGYLPVQTVLEGDNMETPVTLINFWPVDSA
PASSPQLSHDDTHSRIEHYASRLAEMENSNGSYLNDSISPNESIDDEHLLIQHYCQSLNQDSPLSQPRSPAQILI
SLESEERGELERILADLEEENRNLQAEYDRLKQQHEHKGLSPLPSPPEMMPTSPQSP
N-terminus through R1_R17-R19_R24 to 1st coiled coil and proline rich region that follows:
(SEQ ID NO: 123)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRSVEKWRRFHYDIKIFNQWLTEAEQFLRKTQIPENWEHAKYKWYLKELQDGIGQRQTVVRTLNATGEEIIQQSS
KTDASILQEKLGSLNLRWQEVCKQLSDRKKRLEEQKNILSEFQRDLNEFVLWLEEADNIASIPLEPGKEQQLKEK
LEQVKLLVEELPLRQGILKQLNETGGPVLVSAPISPEEQDKLENKLKQTNLQWIKVSRALPEKQGEIEAQIKDLG
QLEKKLEDLEEQLNHLLLWLSPIRNQLEIYNQPNQEGPFDVKETEIAVQAKQPDVEEILSKGQHLYKEKPATQPV
KRKLEDLSSEWKAVNRLLQELRAKQWQRKIDETLERLQELQEATDELDLKLRQAEVIKGSWQPVGDLLIDSLQDH
LEKVKALRGEIAPLKENVSHVNDLARQLTTLGIQLSPYNLSTLEDLNTRWKLLQVAVEDRVRQLHEAHRDFGPAS
QHFLSTSVQGPWERAISPNKVPYYINHETQTTCWDHPKMTELYQSLADLNNVRFSAYRTAMKLRRLQKALCLDLL
SLSAACDALDQHNLKQNDQPMDILQIINCLTTIYDRLEQEHNNLVNVPLCVDMCLNWLLNVYDTGRTGRIRVLSF
KTGIISLCKAHLEDKYRYLFKQVASSTGFCDQRRLGLLLHDSIQIPRQLGEVASFGGSNIEPSVRSCFQFANNKP
EIEAALFLDWMRLEPQSMVWLPVLHRVAAAETAKHQAKCNICKECPIIGFRYRSLKHFNYDICQSCFFSGRVAKG
HKMHYPMVEYCTPTTSGEDVRDFAKVLKNKFRTKRYFAKHPRMGYLPVQTVLEGDNMETPVTLINFWPVDSAPAS
SPQLSHDDTHSRIEHYASRLAEMENSNGSYLNDSISPNESIDDEHLLIQHYCQSLNQDSPLSQPRSPAQILISLE
SEERGELERILADLEEENRNLQAEYDRLKQQHEHKGLSPLPSPPEMMPTSPQSP
N-terminus through R1_R17-R19_H4 to end of dystrophin:
(SEQ ID NO: 5)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRFDRSVEKWRRFHYDIKIFNQWLTEAEQFLRKTQIPENWEHAKYKWYLKELQDGIGQRQTVVRTLNATGEEIIQ
QSSKTDASILQEKLGSLNLRWQEVCKQLSDRKKRLEEQKNILSEFQRDLNEFVLWLEEADNIASIPLEPGKEQQL
KEKLEQVKLLVEELPLRQGILKQLNETGGPVLVSAPISPEEQDKLENKLKQTNLQWIKVSRALPEKQGEIEAQIK
DLGQLEKKLEDLEEQLNHLLLWLSPIRNQLEIYNQPNQEGPFDVKETEIAVQAKQPDVEEILSKGQHLYKEKPAT
QPVKRKLEDLSSEWKAVNRLLQELRAKQGPASQHFLSTSVQGPWERAISPNKVPYYINHETQTTCWDHPKMTELY
QSLADLNNVRFSAYRTAMKLRRLQKALCLDLLSLSAACDALDQHNLKQNDQPMDILQIINCLTTIYDRLEQEHNN
LVNVPLCVDMCLNWLLNVYDTGRTGRIRVLSFKTGIISLCKAHLEDKYRYLFKQVASSTGFCDQRRLGLLLHDSI
QIPRQLGEVASFGGSNIEPSVRSCFQFANNKPEIEAALFLDWMRLEPQSMVWLPVLHRVAAAETAKHQAKCNICK
ECPIIGFRYRSLKHFNYDICQSCFFSGRVAKGHKMHYPMVEYCTPTTSGEDVRDFAKVLKNKFRTKRYFAKHPRM
GYLPVQTVLEGDNMETPVTLINFWPVDSAPASSPQLSHDDTHSRIEHYASRLAEMENSNGSYLNDSISPNESIDD
EHLLIQHYCQSLNQDSPLSQPRSPAQILISLESEERGELERILADLEEENRNLQAEYDRLKQQHEHKGLSPLPSP
PEMMPTSPQSPRDAELIAEAKLLRQHKGRLEARMQILEDHNKQLESQLHRLRQLLEQPQAEAKVNGTTVSSPSTS
LQRSDSSQPMLLRVVGSQTSDSMGEEDLLSPPQDTSTGLEEVMEQLNNSFPSSRGRNTPGKPMREDTM
N-terminus through R1_R17-R19_H4 to end of dystrophin:
(SEQ ID NO: 124)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVITSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRSVEKWRRFHYDIKIFNQWLTEAEQFLRKTQIPENWEHAKYKWYLKELQDGIGQRQTVVRTLNATGEEIIQQSS
KTDASILQEKLGSLNLRWQEVCKQLSDRKKRLEEQKNILSEFQRDLNEFVLWLEEADNIASIPLEPGKEQQLKEK
LEQVKLLVEELPLRQGILKQLNETGGPVLVSAPISPEEQDKLENKLKQTNLQWIKVSRALPEKQGEIEAQIKDLG
QLEKKLEDLEEQLNHLLLWLSPIRNQLEIYNQPNQEGPFDVKETEIAVQAKQPDVEEILSKGQHLYKEKPATQPV
KRKLEDLSSEWKAVNRLLQELRAKQGPASQHFLSTSVQGPWERAISPNKVPYYINHETQTTCWDHPKMTELYQSL
ADLNNVRFSAYRTAMKLRRLQKALCLDLLSLSAACDALDQHNLKQNDQPMDILQIINCLTTIYDRLEQEHNNLVN
VPLCVDMCLNWLLNVYDTGRTGRIRVLSFKTGIISLCKAHLEDKYRYLFKQVASSTGFCDQRRLGLLLHDSIQIP
RQLGEVASFGGSNIEPSVRSCFQFANNKPEIEAALFLDWMRLEPQSMVWLPVLHRVAAAETAKHQAKCNICKECP
IIGFRYRSLKHFNYDICQSCFFSGRVAKGHKMHYPMVEYCTPTTSGEDVRDFAKVLKNKFRTKRYFAKHPRMGYL
PVQTVLEGDNMETPVTLINFWPVDSAPASSPQLSHDDTHSRIEHYASRLAEMENSNGSYLNDSISPNESIDDEHL
LIQHYCQSLNQDSPLSQPRSPAQILISLESEERGELERILADLEEENRNLQAEYDRLKQQHEHKGLSPLPSPPEM
MPTSPQSPRDAELIAEAKLLRQHKGRLEARMQILEDHNKQLESQLHRLRQLLEQPQAEAKVNGTTVSSPSTSLQR
SDSSQPMLLRVVGSQTSDSMGEEDLLSPPQDTSTGLEEVMEQLNNSFPSSRGRNTPGKPMREDTM
N-terminus through R1_R17-R19_R24 to end of dystrophin:
(SEQ ID NO: 6)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRFDRSVEKWRRFHYDIKIFNQWLTEAEQFLRKTQIPENWEHAKYKWYLKELQDGIGQRQTVVRTLNATGEEIIQ
QSSKTDASILQEKLGSLNLRWQEVCKQLSDRKKRLEEQKNILSEFQRDLNEFVLWLEEADNIASIPLEPGKEQQL
KEKLEQVKLLVEELPLRQGILKQLNETGGPVLVSAPISPEEQDKLENKLKQTNLQWIKVSRALPEKQGEIEAQIK
DLGQLEKKLEDLEEQLNHLLLWLSPIRNQLEIYNQPNQEGPFDVKETEIAVQAKQPDVEEILSKGQHLYKEKPAT
QPVKRKLEDLSSEWKAVNRLLQELRAKQWQRKIDETLERLQELQEATDELDLKLRQAEVIKGSWQPVGDLLIDSL
QDHLEKVKALRGEIAPLKENVSHVNDLARQLTTLGIQLSPYNLSTLEDLNTRWKLLQVAVEDRVRQLHEAHRDFG
PASQHFLSTSVQGPWERAISPNKVPYYINHETQTTCWDHPKMTELYQSLADLNNVRFSAYRTAMKLRRLQKALCL
DLLSLSAACDALDQHNLKQNDQPMDILQIINCLTTIYDRLEQEHNNLVNVPLCVDMCLNWLLNVYDTGRTGRIRV
LSFKTGIISLCKAHLEDKYRYLFKQVASSTGFCDQRRLGLLLHDSIQIPRQLGEVASFGGSNIEPSVRSCFQFAN
NKPEIEAALFLDWMRLEPQSMVWLPVLHRVAAAETAKHQAKCNICKECPIIGFRYRSLKHFNYDICQSCFFSGRV
AKGHKMHYPMVEYCTPTTSGEDVRDFAKVLKNKFRTKRYFAKHPRMGYLPVQTVLEGDNMETPVTLINFWPVDSA
PASSPQLSHDDTHSRIEHYASRLAEMENSNGSYLNDSISPNESIDDEHLLIQHYCQSLNQDSPLSQPRSPAQILI
SLESEERGELERILADLEEENRNLQAEYDRLKQQHEHKGLSPLPSPPEMMPTSPQSPRDAELIAEAKLLRQHKGR
LEARMQILEDHNKQLESQLHRLRQLLEQPQAEAKVNGTTVSSPSTSLQRSDSSQPMLLRVVGSQTSDSMGEEDLL
SPPQDTSTGLEEVMEQLNNSFPSSRGRNTPGKPMREDTM
N-terminus through R1_R17-R19_R24 to end of dystrophin:
(SEQ ID NO: 125)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DRSVEKWRRFHYDIKIFNQWLTEAEQFLRKTQIPENWEHAKYKWYLKELQDGIGQRQTVVRTLNATGEEIIQQSS
KTDASILQEKLGSLNLRWQEVCKQLSDRKKRLEEQKNILSEFQRDLNEFVLWLEEADNIASIPLEPGKEQQLKEK
LEQVKLLVEELPLRQGILKQLNETGGPVLVSAPISPEEQDKLENKLKQTNLQWIKVSRALPEKQGEIEAQIKDLG
QLEKKLEDLEEQLNHLLLWLSPIRNQLEIYNQPNQEGPFDVKETEIAVQAKQPDVEEILSKGQHLYKEKPATQPV
KRKLEDLSSEWKAVNRLLQELRAKQWQRKIDETLERLQELQEATDELDLKLRQAEVIKGSWQPVGDLLIDSLQDH
LEKVKALRGEIAPLKENVSHVNDLARQLTTLGIQLSPYNLSTLEDLNTRWKLLQVAVEDRVRQLHEAHRDFGPAS
QHFLSTSVQGPWERAISPNKVPYYINHETQTTCWDHPKMTELYQSLADLNNVRFSAYRTAMKLRRLQKALCLDLL
SLSAACDALDQHNLKQNDQPMDILQIINCLTTIYDRLEQEHNNLVNVPLCVDMCLNWLLNVYDTGRTGRIRVLSF
KTGIISLCKAHLEDKYRYLFKQVASSTGFCDQRRLGLLLHDSIQIPRQLGEVASFGGSNIEPSVRSCFQFANNKP
EIEAALFLDWMRLEPQSMVWLPVLHRVAAAETAKHQAKCNICKECPIIGFRYRSLKHFNYDICQSCFFSGRVAKG
HKMHYPMVEYCTPTTSGEDVRDFAKVLKNKFRTKRYFAKHPRMGYLPVQTVLEGDNMETPVTLINFWPVDSAPAS
SPQLSHDDTHSRIEHYASRLAEMENSNGSYLNDSISPNESIDDEHLLIQHYCQSLNQDSPLSQPRSPAQILISLE
SEERGELERILADLEEENRNLQAEYDRLKQQHEHKGLSPLPSPPEMMPTSPQSPRDAELIAEAKLLRQHKGRLEA
RMQILEDHNKQLESQLHRLRQLLEQPQAEAKVNGTTVSSPSTSLQRSDSSQPMLLRVVGSQTSDSMGEEDLLSPP
QDTSTGLEEVMEQLNNSFPSSRGRNTPGKPMREDTM

### Skeletal Muscle Micro-Dystrophins

The present disclosure provides micro-dystrophin genes and proteins optimized for skeletal muscle. The inventors have found that certain combinations of domains from full-length dystrophin are beneficial for skeletal muscle.

In some embodiments, a micro-dystrophin protein optimized for skeletal muscle does not comprise spectrin-like repeat 2 and/or spectrin-like repeat 3 of dystrophin. In some embodiments, a micro-dystrophin protein does not comprise spectrin-like repeat 2. In some embodiments, a micro-dystrophin protein does not comprise spectrin-like repeat 3. In some embodiments, a micro-dystrophin protein does not comprise spectrin-like repeat 2 and spectrin-like repeat 3.

In some embodiments, a micro-dystrophin protein comprises optimized for skeletal muscle spectrin-like repeat 1 and spectrin-like repeat 2. In some embodiments, a micro-dystrophin protein optimized for skeletal muscle comprises spectrin-like repeat 1, spectrin-like repeat 2, and spectrin-like repeat 3.

In some embodiments, a micro-dystrophin protein optimized for skeletal muscle comprises an amino-terminal actin-binding domain, hinge domain 1, spectrin-like repeat 1, spectrin-like repeat 2, spectrin-like repeat 16, and spectrin-like repeat 17 of dystrophin. In some embodiments, the micro-dystrophin protein does not comprise spectrin-like repeat 3 of dystrophin. In some embodiments, the micro-dystrophin protein comprises spectrin-like repeat 3 of dystrophin.

In some embodiments, a micro-dystrophin protein optimized for skeletal muscle comprises one or more of: spectrin-like repeat 23, spectrin-like repeat 24, hinge domain 4, and the dystroglycan binding site of dystrophin. In some embodiments, a micro-dystrophin protein optimized for skeletal muscle comprises spectrin-like repeat 23 and spectrin-like repeat 24. In some embodiments, a micro-dystrophin protein optimized for skeletal muscle comprises spectrin-like repeat 23 and hinge domain 4. In some embodiments, a micro-dystrophin protein optimized for skeletal muscle comprises spectrin-like repeat 23 and the dystroglycan binding site. In some embodiments, a micro-dystrophin protein optimized for skeletal muscle comprises spectrin-like repeat 24 and hinge domain 4. In some embodiments, a micro-dystrophin protein optimized for skeletal muscle comprises spectrin-like repeat 24 and the dystroglycan binding site. In some embodiments, a micro-dystrophin protein optimized for skeletal muscle comprises hinge domain 4 and the dystroglycan binding site. In some embodiments, a micro-dystrophin protein optimized for skeletal muscle comprises spectrin-like repeat 23, spectrin-like repeat 24 and hinge domain 4. In some embodiments, a micro-dystrophin protein optimized for skeletal muscle comprises spectrin-like repeat 23, spectrin-like repeat 24 and the dystroglycan binding site. In some embodiments, a micro-dystrophin protein optimized for skeletal muscle comprises spectrin-like repeat 23, hinge domain 4, and the dystroglycan binding site. In some embodiments, a micro-dystrophin protein optimized for skeletal muscle comprises spectrin-like repeat 24, hinge domain 4, and the dystroglycan binding site. In some embodiments, a micro-dystrophin protein optimized for skeletal muscle comprises spectrin-like repeat 23, spectrin-like repeat 24, hinge domain 4, and the dystroglycan binding site.

In some embodiments, a micro-dystrophin protein optimized for skeletal muscle comprises one or more syntrophin binding domains of dystrophin.

In some embodiments, a micro-dystrophin protein optimized for skeletal muscle comprises one or more coiled coil domains of dystrophin.

In some embodiments, a micro-dystrophin protein optimized for skeletal muscle comprises spectrin-like repeat 2 directly coupled to spectrin like repeat 16.

In some embodiments, a micro-dystrophin protein optimized for skeletal muscle comprises spectrin-like repeat 2 directly coupled to spectrin like repeat 23.

In some embodiments, a micro-dystrophin protein optimized for skeletal muscle comprises spectrin-like repeat 1 directly coupled spectrin-like repeat 15.

In some embodiments, a micro-dystrophin protein optimized for skeletal muscle comprises spectrin-like repeat 17 directly coupled to spectrin-like repeat 24. In some embodiments, a micro-dystrophin protein optimized for skeletal muscle comprises spectrin-like repeat 17 directly coupled to hinge domain 4.

In some embodiments, a micro-dystrophin protein optimized for skeletal muscle comprises the region of dystrophin that is C-terminal to the cysteine-rich portion of the C-terminal region (FIG. 2). In some embodiments, a micro-dystrophin optimized for skeletal muscle comprises region of dystrophin that is C-terminal to the dystroglycan binding site of dystrophin.

In some embodiments, a micro-dystrophin protein optimized for skeletal muscle comprises the region of dystrophin from spectrin-like repeat 24 to the C-terminus. In some embodiments, a micro-dystrophin protein optimized for skeletal muscle comprises the region of dystrophin from spectrin-like repeat 24 to the end of the proline rich region following the first coiled coil domain.

In some embodiments, a micro-dystrophin protein optimized for skeletal muscle comprises the region of dystrophin from hinge domain 4 to the C-terminus. In some embodiments, a micro-dystrophin protein optimized for skeletal muscle comprises the region of dystrophin from hinge domain 4 to the end of the proline rich region following the first coiled coil domain. In some embodiments, a micro-dystrophin protein optimized for skeletal muscle comprises the region of dystrophin from hinge domain 4 to the end of the second syntrophin binding domain.

Non-limiting examples of human micro-dystrophin proteins optimized for skeletal muscle are described below.

Nterm-R2_R16-R17_R24 + H4 + trCterm:
(SEQ ID NO: 13)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHTVREETMMVMTEDMPLEISYVPSTYLTEITHVSQALLEVEQLL
NAPDLCAKDFEDLFKQEESLKNIKDSLQQSSGRIDIIHSKKTAALQSATPVERVKLQEALSQLDFQWEKVNKMYK
DRQGRFDRSVEKWRRFHYDIKIFNQWLTEAEQFLRKTQIPENWEHAKYKWYLKELQDGIGQRQTVVRTLNATGEE
IIQQSSKTDASILQEKLGSLNLRWQEVCKQLSDRKKRLEEQKWQRKIDETLERLQELQEATDELDLKLRQAEVIK
GSWQPVGDLLIDSLQDHLEKVKALRGEIAPLKENVSHVNDLARQLTTLGIQLSPYNLSTLEDLNTRWKLLQVAVE
DRVRQLHEAHRDFGPASQHFLSTSVQGPWERAISPNKVPYYINHETQTTCWDHPKMTELYQSLADLNNVRFSAYR
TAMKLRRLQKALCLDLLSLSAACDALDQHNLKQNDQPMDILQIINCLTTIYDRLEQEHNNLVNVPLCVDMCLNWL
LNVYDTGRTGRIRVLSFKTGIISLCKAHLEDKYRYLFKQVASSTGFCDQRRLGLLLHDSIQIPRQLGEVASFGGS
NIEPSVRSCFQFANNKPEIEAALFLDWMRLEPQSMVWLPVLHRVAAAETAKHQAKCNICKECPIIGFRYRSLKHF
NYDICQSCFFSGRVAKGHKMHYPMVEYCTPTTSGEDVRDFAKVLKNKFRTKRYFAKHPRMGYLPVQTVLEGDNME
TP
Nterm-R2_R16-R17_H4 to end of syntrophin region:
(SEQ ID NO: 14)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHTVREETMMVMTEDMPLEISYVPSTYLTEITHVSQALLEVEQLL
NAPDLCAKDFEDLFKQEESLKNIKDSLQQSSGRIDIIHSKKTAALQSATPVERVKLQEALSQLDFQWEKVNKMYK
DRQGRFDRSVEKWRRFHYDIKIFNQWLTEAEQFLRKTQIPENWEHAKYKWYLKELQDGIGQRQTVVRTLNATGEE
IIQQSSKTDASILQEKLGSLNLRWQEVCKQLSDRKKRLEEQKGPASQHFLSTSVQGPWERAISPNKVPYYINHET
QTTCWDHPKMTELYQSLADLNNVRFSAYRTAMKLRRLQKALCLDLLSLSAACDALDQHNLKQNDQPMDILQIINC
LTTIYDRLEQEHNNLVNVPLCVDMCLNWLLNVYDTGRTGRIRVLSFKTGIISLCKAHLEDKYRYLFKQVASSTGF
CDQRRLGLLLHDSIQIPRQLGEVASFGGSNIEPSVRSCFQFANNKPEIEAALFLDWMRLEPQSMVWLPVLHRVAA
AETAKHQAKCNICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETPVTLINFWPVDSAPASSPQLSHDDTHSRIEHYASRLAEMENSNGS
YLNDSISPNESIDDEHLLIQHYCQSLNQDSPLSQPRS
Nterm-R2_R16-R17_H4 to 1st coiled coil and proline rich region that follows:
(SEQ ID NO: 15)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHTVREETMMVMTEDMPLEISYVPSTYLTEITHVSQALLEVEQLL
NAPDLCAKDFEDLFKQEESLKNIKDSLQQSSGRIDIIHSKKTAALQSATPVERVKLQEALSQLDFQWEKVNKMYK
DRQGRFDRSVEKWRRFHYDIKIFNQWLTEAEQFLRKTQIPENWEHAKYKWYLKELQDGIGQRQTVVRTLNATGEE
IIQQSSKTDASILQEKLGSLNLRWQEVCKQLSDRKKRLEEQKGPASQHFLSTSVQGPWERAISPNKVPYYINHET
QTTCWDHPKMTELYQSLADLNNVRFSAYRTAMKLRRLQKALCLDLLSLSAACDALDQHNLKQNDQPMDILQIINC
LTTIYDRLEQEHNNLVNVPLCVDMCLNWLLNVYDTGRTGRIRVLSFKTGIISLCKAHLEDKYRYLFKQVASSTGF
CDQRRLGLLLHDSIQIPRQLGEVASFGGSNIEPSVRSCFQFANNKPEIEAALFLDWMRLEPQSMVWLPVLHRVAA
AETAKHQAKCNICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETPVTLINFWPVDSAPASSPQLSHDDTHSRIEHYASRLAEMENSNGS
YLNDSISPNESIDDEHLLIQHYCQSLNQDSPLSQPRSPAQILISLESEERGELERILADLEEENRNLQAEYDRLK
QQHEHKGLSPLPSPPEMMPTSPQSP
Nterm-R2_R16-R17_R24 to 1st coiled coil and proline rich region that follows:
(SEQ ID NO: 16)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHTVREETMMVMTEDMPLEISYVPSTYLTEITHVSQALLEVEQLL
NAPDLCAKDFEDLFKQEESLKNIKDSLQQSSGRIDIIHSKKTAALQSATPVERVKLQEALSQLDFQWEKVNKMYK
DRQGRFDRSVEKWRRFHYDIKIFNQWLTEAEQFLRKTQIPENWEHAKYKWYLKELQDGIGQRQTVVRTLNATGEE
IIQQSSKTDASILQEKLGSLNLRWQEVCKQLSDRKKRLEEQKWQRKIDETLERLQELQEATDELDLKLRQAEVIK
GSWQPVGDLLIDSLQDHLEKVKALRGEIAPLKENVSHVNDLARQLTTLGIQLSPYNLSTLEDLNTRWKLLQVAVE
DRVRQLHEAHRDFGPASQHFLSTSVQGPWERAISPNKVPYYINHETQTTCWDHPKMTELYQSLADLNNVRFSAYR
TAMKLRRLQKALCLDLLSLSAACDALDQHNLKQNDQPMDILQIINCLTTIYDRLEQEHNNLVNVPLCVDMCLNWL
LNVYDTGRTGRIRVLSFKTGIISLCKAHLEDKYRYLFKQVASSTGFCDQRRLGLLLHDSIQIPRQLGEVASFGGS
NIEPSVRSCFQFANNKPEIEAALFLDWMRLEPQSMVWLPVLHRVAAAETAKHQAKCNICKECPIIGFRYRSLKHF
NYDICQSCFFSGRVAKGHKMHYPMVEYCTPTTSGEDVRDFAKVLKNKFRTKRYFAKHPRMGYLPVQTVLEGDNME
TPVTLINFWPVDSAPASSPQLSHDDTHSRIEHYASRLAEMENSNGSYLNDSISPNESIDDEHLLIQHYCQSLNQD
SPLSQPRSPAQILISLESEERGELERILADLEEENRNLQAEYDRLKQQHEHKGLSPLPSPPEMMPTSPQSP
Nterm-R2_R23_R24 to end of dystrophin:
(SEQ ID NO: 17)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHLSLQELLVWLQLKDDELSRQAPIGGDFPAVQKQNDVHRAFKRE
LKTKEPVIMSTLETVRIFLTEQPLEGLEKLYQEPRELPPEERAQNVTRLLRKQAEEVNTEWEKLNLHSADWQRKI
DETLERLQELQEATDELDLKLRQAEVIKGSWQPVGDLLIDSLQDHLEKVKALRGEIAPLKENVSHVNDLARQLTT
LGIQLSPYNLSTLEDLNTRWKLLQVAVEDRVRQLHEAHRDFGPASQHFLSTSVQGPWERAISPNKVPYYINHETQ
TTCWDHPKMTELYQSLADLNNVRFSAYRTAMKLRRLQKALCLDLLSLSAACDALDQHNLKQNDQPMDILQIINCL
TTIYDRLEQEHNNLVNVPLCVDMCLNWLLNVYDTGRTGRIRVLSFKTGIISLCKAHLEDKYRYLFKQVASSTGFC
DQRRLGLLLHDSIQIPRQLGEVASFGGSNIEPSVRSCFQFANNKPEIEAALFLDWMRLEPQSMVWLPVLHRVAAA
ETAKHQAKCNICKECPIIGFRYRSLKHFNYDICQSCFFSGRVAKGHKMHYPMVEYCTPTTSGEDVRDFAKVLKNK
FRTKRYFAKHPRMGYLPVQTVLEGDNMETPVTLINFWPVDSAPASSPQLSHDDTHSRIEHYASRLAEMENSNGSY
LNDSISPNESIDDEHLLIQHYCQSLNQDSPLSQPRSPAQILISLESEERGELERILADLEEENRNLQAEYDRLKQ
QHEHKGLSPLPSPPEMMPTSPQSPRDAELIAEAKLLRQHKGRLEARMQILEDHNKQLESQLHRLRQLLEQPQAEA
KVNGTTVSSPSTSLQRSDSSQPMLLRVVGSQTSDSMGEEDLLSPPQDTSTGLEEVMEQLNNSFPSSRGRNTPGKP
MREDTM
Nterm-R2_R23_R24 to end of dystrophin:
(SEQ ID NO: 132)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDASSDQWKRLHLSLQELLVWLQLKDDELSRQAPIGGDFPAVQKQN
DVHRAFKRELKTKEPVIMSTLETVRIFLTEQPLEGLEKLYQEPRELPPEERAQNVTRLLRKQAEEVNTEWEKLNL
HSADWQRKIDETLERLQELQEATDELDLKLRQAEVIKGSWQPVGDLLIDSLQDHLEKVKALRGEIAPLKENVSHV
NDLARQLTTLGIQLSPYNLSTLEDLNTRWKLLQVAVEDRVRQLHEAHRDFGPASQHFLSTSVQGPWERAISPNKV
PYYINHETQTTCWDHPKMTELYQSLADLNNVRFSAYRTAMKLRRLQKALCLDLLSLSAACDALDQHNLKQNDQPM
DILQIINCLTTIYDRLEQEHNNLVNVPLCVDMCLNWLLNVYDTGRTGRIRVLSFKTGIISLCKAHLEDKYRYLFK
QVASSTGFCDQRRLGLLLHDSIQIPRQLGEVASFGGSNIEPSVRSCFQFANNKPEIEAALFLDWMRLEPQSMVWL
PVLHRVAAAETAKHQAKCNICKECPIIGFRYRSLKHFNYDICQSCFFSGRVAKGHKMHYPMVEYCTPTTSGEDVR
DFAKVLKNKFRTKRYFAKHPRMGYLPVQTVLEGDNMETPVTLINFWPVDSAPASSPQLSHDDTHSRIEHYASRLA
EMENSNGSYLNDSISPNESIDDEHLLIQHYCQSLNQDSPLSQPRSPAQILISLESEERGELERILADLEEENRNL
QAEYDRLKQQHEHKGLSPLPSPPEMMPTSPQSPRDAELIAEAKLLRQHKGRLEARMQILEDHNKQLESQLHRLRQ
LLEQPQAEAKVNGTTVSSPSTSLQRSDSSQPMLLRVVGSQTSDSMGEEDLLSPPQDTSTGLEEVMEQLNNSFPSS
RGRNTPGKPMREDTM
Nterm-R2_R16-R17_H4 to end:
(SEQ ID NO: 18)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHTVREETMMVMTEDMPLEISYVPSTYLTEITHVSQALLEVEQLL
NAPDLCAKDFEDLFKQEESLKNIKDSLQQSSGRIDIIHSKKTAALQSATPVERVKLQEALSQLDFQWEKVNKMYK
DRQGRFDRSVEKWRRFHYDIKIFNQWLTEAEQFLRKTQIPENWEHAKYKWYLKELQDGIGQRQTVVRTLNATGEE
IIQQSSKTDASILQEKLGSLNLRWQEVCKQLSDRKKRLEEQKGPASQHFLSTSVQGPWERAISPNKVPYYINHET
QTTCWDHPKMTELYQSLADLNNVRFSAYRTAMKLRRLQKALCLDLLSLSAACDALDQHNLKQNDQPMDILQIINC
LTTIYDRLEQEHNNLVNVPLCVDMCLNWLLNVYDTGRTGRIRVLSFKTGIISLCKAHLEDKYRYLFKQVASSTGF
CDQRRLGLLLHDSIQIPRQLGEVASFGGSNIEPSVRSCFQFANNKPEIEAALFLDWMRLEPQSMVWLPVLHRVAA
AETAKHQAKCNICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETPVTLINFWPVDSAPASSPQLSHDDTHSRIEHYASRLAEMENSNGS
YLNDSISPNESIDDEHLLIQHYCQSLNQDSPLSQPRSPAQILISLESEERGELERILADLEEENRNLQAEYDRLK
QQHEHKGLSPLPSPPEMMPTSPQSPRDAELIAEAKLLRQHKGRLEARMQILEDHNKQLESQLHRLRQLLEQPQAE
AKVNGTTVSSPSTSLQRSDSSQPMLLRVVGSQTSDSMGEEDLLSPPQDTSTGLEEVMEQLNNSFPSSRGRNTPGK
PMREDTM
Nterm-R1_R16-R17_R24 to end:
(SEQ ID NO: 19)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DHTVREETMMVMTEDMPLEISYVPSTYLTEITHVSQALLEVEQLLNAPDLCAKDFEDLFKQEESLKNIKDSLQQS
SGRIDIIHSKKTAALQSATPVERVKLQEALSQLDFQWEKVNKMYKDRQGRFDRSVEKWRRFHYDIKIFNQWLTEA
EQFLRKTQIPENWEHAKYKWYLKELQDGIGQRQTVVRTLNATGEEIIQQSSKTDASILQEKLGSLNLRWQEVCKQ
LSDRKKRLEEQKWQRKIDETLERLQELQEATDELDLKLRQAEVIKGSWQPVGDLLIDSLQDHLEKVKALRGEIAP
LKENVSHVNDLARQLTTLGIQLSPYNLSTLEDLNTRWKLLQVAVEDRVRQLHEAHRDFGPASQHFLSTSVQGPWE
RAISPNKVPYYINHETQTTCWDHPKMTELYQSLADLNNVRFSAYRTAMKLRRLQKALCLDLLSLSAACDALDQHN
LKQNDQPMDILQIINCLTTIYDRLEQEHNNLVNVPLCVDMCLNWLLNVYDTGRTGRIRVLSFKTGIISLCKAHLE
DKYRYLFKQVASSTGFCDQRRLGLLLHDSIQIPRQLGEVASFGGSNIEPSVRSCFQFANNKPEIEAALFLDWMRL
EPQSMVWLPVLHRVAAAETAKHQAKCNICKECPIIGFRYRSLKHFNYDICQSCFFSGRVAKGHKMHYPMVEYCTP
TTSGEDVRDFAKVLKNKFRTKRYFAKHPRMGYLPVQTVLEGDNMETPVTLINFWPVDSAPASSPQLSHDDTHSRI
EHYASRLAEMENSNGSYLNDSISPNESIDDEHLLIQHYCQSLNQDSPLSQPRSPAQILISLESEERGELERILAD
LEEENRNLQAEYDRLKQQHEHKGLSPLPSPPEMMPTSPQSPRDAELIAEAKLLRQHKGRLEARMQILEDHNKQLE
SQLHRLRQLLEQPQAEAKVNGTTVSSPSTSLQRSDSSQPMLLRVVGSQTSDSMGEEDLLSPPQDTSTGLEEVMEQ
LNNSFPSSRGRNTPGKPMREDTM
Nterm-R2_R16-R17_R24 to end:
(SEQ ID NO: 20)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHTVREETMMVMTEDMPLEISYVPSTYLTEITHVSQALLEVEQLL
NAPDLCAKDFEDLFKQEESLKNIKDSLQQSSGRIDIIHSKKTAALQSATPVERVKLQEALSQLDFQWEKVNKMYK
DRQGRFDRSVEKWRRFHYDIKIFNQWLTEAEQFLRKTQIPENWEHAKYKWYLKELQDGIGQRQTVVRTLNATGEE
IIQQSSKTDASILQEKLGSLNLRWQEVCKQLSDRKKRLEEQKWQRKIDETLERLQELQEATDELDLKLRQAEVIK
GSWQPVGDLLIDSLQDHLEKVKALRGEIAPLKENVSHVNDLARQLTTLGIQLSPYNLSTLEDLNTRWKLLQVAVE
DRVRQLHEAHRDFGPASQHFLSTSVQGPWERAISPNKVPYYINHETQTTCWDHPKMTELYQSLADLNNVRFSAYR
TAMKLRRLQKALCLDLLSLSAACDALDQHNLKQNDQPMDILQIINCLTTIYDRLEQEHNNLVNVPLCVDMCLNWL
LNVYDTGRTGRIRVLSFKTGIISLCKAHLEDKYRYLFKQVASSTGFCDQRRLGLLLHDSIQIPRQLGEVASFGGS
NIEPSVRSCFQFANNKPEIEAALFLDWMRLEPQSMVWLPVLHRVAAAETAKHQAKCNICKECPIIGFRYRSLKHF
NYDICQSCFFSGRVAKGHKMHYPMVEYCTPTTSGEDVRDFAKVLKNKFRTKRYFAKHPRMGYLPVQTVLEGDNME
TPVTLINFWPVDSAPASSPQLSHDDTHSRIEHYASRLAEMENSNGSYLNDSISPNESIDDEHLLIQHYCQSLNQD
SPLSQPRSPAQILISLESEERGELERILADLEEENRNLQAEYDRLKQQHEHKGLSPLPSPPEMMPTSPQSPRDAE
LIAEAKLLRQHKGRLEARMQILEDHNKQLESQLHRLRQLLEQPQAEAKVNGTTVSSPSTSLQRSDSSQPMLLRVV
GSQTSDSMGEEDLLSPPQDTSTGLEEVMEQLNNSFPSSRGRNTPGKPMREDTM
Nterm-R2_R16-R17_R24 to end of Syntrophin region:
(SEQ ID NO: 21)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEEVLSWLLSAEDTLQAQGEISNDVEVV
KDQFHTHEGYMMDLTAHQGRVGNILQLGSKLIGTGKLSEDEETEVQEQMNLLNSRWECLRVASMEKQSNLHRVLM
DLQNQKLKELNDWLTKTEERTRKMEEEPLGPDLEDLKRQVQQHKVLQEDLEQEQVRVNSLTHMVVVVDESSGDHA
TAALEEQLKVLGDRWANICRWTEDRWVLLQDHTVREETMMVMTEDMPLEISYVPSTYLTEITHVSQALLEVEQLL
NAPDLCAKDFEDLFKQEESLKNIKDSLQQSSGRIDIIHSKKTAALQSATPVERVKLQEALSQLDFQWEKVNKMYK
DRQGRFDRSVEKWRRFHYDIKIFNQWLTEAEQFLRKTQIPENWEHAKYKWYLKELQDGIGQRQTVVRTLNATGEE
IIQQSSKTDASILQEKLGSLNLRWQEVCKQLSDRKKRLEEQKWQRKIDETLERLQELQEATDELDLKLRQAEVIK
GSWQPVGDLLIDSLQDHLEKVKALRGEIAPLKENVSHVNDLARQLTTLGIQLSPYNLSTLEDLNTRWKLLQVAVE
DRVRQLHEAHRDFGPASQHFLSTSVQGPWERAISPNKVPYYINHETQTTCWDHPKMTELYQSLADLNNVRFSAYR
TAMKLRRLQKALCLDLLSLSAACDALDQHNLKQNDQPMDILQIINCLTTIYDRLEQEHNNLVNVPLCVDMCLNWL
LNVYDTGRTGRIRVLSFKTGIISLCKAHLEDKYRYLFKQVASSTGFCDQRRLGLLLHDSIQIPRQLGEVASFGGS
NIEPSVRSCFQFANNKPEIEAALFLDWMRLEPQSMVWLPVLHRVAAAETAKHQAKCNICKECPIIGFRYRSLKHF
NYDICQSCFFSGRVAKGHKMHYPMVEYCTPTTSGEDVRDFAKVLKNKFRTKRYFAKHPRMGYLPVQTVLEGDNME
TPVTLINFWPVDSAPASSPQLSHDDTHSRIEHYASRLAEMENSNGSYLNDSISPNESIDDEHLLIQHYCQSLNQD
SPLSQPRS

A micro-dystrophin protein optimized for skeletal muscle is further understood to include proteins that are variants of any one of SEQ ID Nos. 13-21, 93-101, 132, and 149-155. Variant polypeptides include polypeptides that differ by one or more (e.g., 1, 2, 3, 4, 5, 6, 7, 8, 9, 10, 11, 12, 13, 14, 15, 16, 17, 18, 19, 20, or more) amino acid substitutions, additions, or deletions, and will, therefore, include amino acid sequences that differ from the amino acid sequences designated in any one of SEQ ID Nos. 13-21, 93-101, 132, and 149-155.

In some embodiments, a micro-dystrophin protein optimized for skeletal muscle comprises an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 13-21, 93-101, 132, and 149-155. In some embodiments, a micro-dystrophin protein optimized for skeletal muscle consists essentially of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 13-21, 93-101, 132, and 149-155.

In some embodiments, a micro-dystrophin protein optimized for skeletal muscle consists of an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 13-21, 93-101, 132, and 149-155.

In some embodiments, functional variants or modified forms of micro-dystrophin proteins optimized for skeletal muscle include fusion proteins having at least a portion of the micro-dystrophin protein and one or more fusion domains. Well-known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. Other fusion domains are particularly useful for increasing protein stability.

The disclosure also provides nucleic acids encoding any of the micro-dystrophins optimized for skeletal muscle described herein. Such nucleic acids may be DNA or RNA molecules. These nucleic acids may be used, for example, in methods for making micro-dystrophins or as direct therapeutic agents in a gene therapy approach.

In some embodiments, a micro-dystrophin protein as described herein further comprises one or more utrophin protein domains.

Non-limiting examples of nucleotide sequences encoding human micro-dystrophin proteins optimized for skeletal muscle are described below.

Nterm-R2_R16-R17_R24 + H4 + trCterm:
(SEQ ID NO: 22)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACACTGTCCGTGAAGAAACGATGATGGTGATGACTGAAGACATGCCTTTGGAAATT
TCTTATGTGCCTTCTACTTATTTGACTGAAATCACTCATGTCTCACAAGCCCTATTAGAAGTGGAACAACTTCTC
AATGCTCCTGACCTCTGTGCTAAGGACTTTGAAGATCTCTTTAAGCAAGAGGAGTCTCTGAAGAATATAAAAGAT
AGTCTACAACAAAGCTCAGGTCGGATTGACATTATTCATAGCAAGAAGACAGCAGCATTGCAAAGTGCAACGCCT
GTGGAAAGGGTGAAGCTACAGGAAGCTCTCTCCCAGCTTGATTTCCAATGGGAAAAAGTTAACAAAATGTACAAG
GACCGACAAGGGCGATTTGACAGATCTGTTGAGAAATGGCGGCGTTTTCATTATGATATAAAGATATTTAATCAG
TGGCTAACAGAAGCTGAACAGTTTCTCAGAAAGACACAAATTCCTGAGAATTGGGAACATGCTAAATACAAATGG
TATCTTAAGGAACTCCAGGATGGCATTGGGCAGCGGCAAACTGTTGTCAGAACATTGAATGCAACTGGGGAAGAA
ATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGTGGCAGAGAAAAATAGATGAGACC
CTTGAAAGACTCCGGGAACTTCAAGAGGCCACGGATGAGCTGGACCTCAAGCTGCGCCAAGCTGAGGTGATCAAG
GGATCCTGGCAGCCCGTGGGCGATCTCCTCATTGACTCTCTCCAAGATCACCTCGAGAAAGTCAAGGCACTTCGA
GGAGAAATTGCGCCTCTGAAAGAGAACGTGAGCCACGTCAATGACCTTGCTCGCCAGCTTACCACTTTGGGCATT
CAGCTCTCACCGTATAACCTCAGCACTCTGGAAGACCTGAACACCAGATGGAAGCTTCTGCAGGTGGCCGTCGAG
GACCGAGTCAGGCAGCTGCATGAAGCCCACAGGGACTTTGGTCCAGCATCTCAGCACTTTCTTTCCACGTCTGTC
CAGGGTCCCTGGGAGAGAGCCATCTCGCCAAACAAAGTGCCCTACTATATCAACCACGAGACTCAAACAACTTGC
TGGGACCATCCCAAAATGACAGAGCTCTACCAGTCTTTAGCTGACCTGAATAATGTCAGATTCTCAGCTTATAGG
ACTGCCATGAAACTCCGAAGACTGCAGAAGGCCCTTTGCTTGGATCTCTTGAGCCTGTCAGCTGCATGTGATGCC
TTGGACCAGCACAACCTCAAGCAAAATGACCAGCCCATGGATATCCTGCAGATTATTAATTGTTTGACCACTATT
TATGACCGCCTGGAGCAAGAGCACAACAATTTGGTCAACGTCCCTCTCTGCGTGGATATGTGTCTGAACTGGCTG
CTGAATGTTTATGATACGGGACGAACAGGGAGGATCCGTGTCCTGTCTTTTAAAACTGGCATCATTTCCCTGTGT
AAAGCACATTTGGAAGACAAGTACAGATACCTTTTCAAGCAAGTGGCAAGTTCAACAGGATTTTGTGACCAGCGC
AGGCTGGGCCTCCTTCTGCATGATTCTATCCAAATTCCAAGACAGTTGGGTGAAGTTGCATCCTTTGGGGGCAGT
AACATTGAGCCAAGTGTCCGGAGCTGCTTCCAATTTGCTAATAATAAGCCAGAGATCGAAGCGGCCCTCTTCCTA
GACTGGATGAGACTGGAACCCCAGTCCATGGTGTGGCTGCCCGTCCTGCACAGAGTGGCTGCTGCAGAAACTGCC
AAGCATCAGGCCAAATGTAACATCTGCAAAGAGTGTCCAATCATTGGATTCAGGTACAGGAGTCTAAAGCACTTT
AATTATGACATCTGCCAAAGCTGCTTTTTTTCTGGTCGAGTTGCAAAAGGCCATAAAATGCACTATCCCATGGTG
GAATATTGCACTCCGACTACATCAGGAGAAGATGTTCGAGACTTTGCCAAGGTACTAAAAAACAAATTTCGAACC
AAAAGGTATTTTGCGAAGCATCCCCGAATGGGCTACCTGCCAGTGCAGACTGTCTTAGAGGGGGACAACATGGAA
ACTCCC
Nterm-R2_R16-R17_H4 to end of syntrophin region:
(SEQ ID NO: 23)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACACTGTCCGTGAAGAAACGATGATGGTGATGACTGAAGACATGCCTTTGGAAATT
TCTTATGTGCCTTCTACTTATTTGACTGAAATCACTCATGTCTCACAAGCCCTATTAGAAGTGGAACAACTTCTC
AATGCTCCTGACCTCTGTGCTAAGGACTTTGAAGATCTCTTTAAGCAAGAGGAGTCTCTGAAGAATATAAAAGAT
AGTCTACAACAAAGCTCAGGTCGGATTGACATTATTCATAGCAAGAAGACAGCAGCATTGCAAAGTGCAACGCCT
GTGGAAAGGGTGAAGCTACAGGAAGCTCTCTCCCAGCTTGATTTCCAATGGGAAAAAGTTAACAAAATGTACAAG
GACCGACAAGGGCGATTTGACAGATCTGTTGAGAAATGGCGGCGTTTTCATTATGATATAAAGATATTTAATCAG
TGGCTAACAGAAGCTGAACAGTTTCTCAGAAAGACACAAATTCCTGAGAATTGGGAACATGCTAAATACAAATGG
TATCTTAAGGAACTCCAGGATGGCATTGGGCAGCGGCAAACTGTTGTCAGAACATTGAATGCAACTGGGGAAGAA
ATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGGGTCCAGCATCTCAGCACTTTCTT
TCCACGTCTGTCCAGGGTCCCTGGGAGAGAGCCATCTCGCCAAACAAAGTGCCCTACTATATCAACCACGAGACT
CAAACAACTTGCTGGGACCATCCCAAAATGACAGAGCTCTACCAGTCTTTAGCTGACCTGAATAATGTCAGATTC
TCAGCTTATAGGACTGCCATGAAACTCCGAAGACTGCAGAAGGCCCTTTGCTTGGATCTCTTGAGCCTGTCAGCT
GCATGTGATGCCTTGGACCAGCACAACCTCAAGCAAAATGACCAGCCCATGGATATCCTGCAGATTATTAATTGT
TTGACCACTATTTATGACCGCCTGGAGCAAGAGCACAACAATTTGGTCAACGTCCCTCTCTGCGTGGATATGTGT
CTGAACTGGCTGCTGAATGTTTATGATACGGGACGAACAGGGAGGATCCGTGTCCTGTCTTTTAAAACTGGCATC
ATTTCCCTGTGTAAAGCACATTTGGAAGACAAGTACAGATACCTTTTCAAGCAAGTGGCAAGTTCAACAGGATTT
TGTGACCAGCGCAGGCTGGGCCTCCTTCTGCATGATTCTATCCAAATTCCAAGACAGTTGGGTGAAGTTGCATCC
TTTGGGGGCAGTAACATTGAGCCAAGTGTCCGGAGCTGCTTCCAATTTGCTAATAATAAGCCAGAGATCGAAGCG
GCCCTCTTCCTAGACTGGATGAGACTGGAACCCCAGTCCATGGTGTGGCTGCCCGTCCTGCACAGAGTGGCTGCT
GCAGAAACTGCCAAGCATCAGGCCAAATGTAACATCTGCAAAGAGTGTCCAATCATTGGATTCAGGTACAGGAGT
CTAAAGCACTTTAATTATGACATCTGCCAAAGCTGCTTTTTTTCTGGTCGAGTTGCAAAAGGCCATAAAATGCAC
TATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGG
GACAACATGGAAACTCCCGTTACTCTGATCAACTTCTGGCCAGTAGATTCTGCGCCTGCCTCGTCCCCTCAGCTT
TCACACGATGATACTCATTCACGCATTGAACATTATGCTAGCAGGCTAGCAGAAATGGAAAACAGCAATGGATCT
TATCTAAATGATAGCATCTCTCCTAATGAGAGCATAGATGATGAACATTTGTTAATCCAGCATTACTGCCAAAGT
TTGAACCAGGACTCCCCCCTGAGCCAGCCTCGTAGT
Nterm-R2_R16-R17_H4 to 1st coiled coil and proline rich region that follows:
(SEQ ID NO: 24)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACACTGTCCGTGAAGAAACGATGATGGTGATGACTGAAGACATGCCTTTGGAAATT
TCTTATGTGCCTTCTACTTATTTGACTGAAATCACTCATGTCTCACAAGCCCTATTAGAAGTGGAACAACTTCTC
AATGCTCCTGACCTCTGTGCTAAGGACTTTGAAGATCTCTTTAAGCAAGAGGAGTCTCTGAAGAATATAAAAGAT
AGTCTACAACAAAGCTCAGGTCGGATTGACATTATTCATAGCAAGAAGACAGCAGCATTGCAAAGTGCAACGCCT
GTGGAAAGGGTGAAGCTACAGGAAGCTCTCTCCCAGCTTGATTTCCAATGGGAAAAAGTTAACAAAATGTACAAG
GACCGACAAGGGCGATTTGACAGATCTGTTGAGAAATGGCGGCGTTTTCATTATGATATAAAGATATTTAATCAG
TGGCTAACAGAAGCTGAACAGTTTCTCAGAAAGACACAAATTCCTGAGAATTGGGAACATGCTAAATACAAATGG
TATCTTAAGGAACTCCAGGATGGCATTGGGCAGCGGCAAACTGTTGTCAGAACATTGAATGCAACTGGGGAAGAA
ATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGGGTCCAGCATCTCAGCACTTTCTT
TCCACGTCTGTCCAGGGTCCCTGGGAGAGAGCCATCTCGCCAAACAAAGTGCCCTACTATATCAACCACGAGACT
CAAACAACTTGCTGGGACCATCCCAAAATGACAGAGCTCTACCAGTCTTTAGCTGACCTGAATAATGTCAGATTC
TCAGCTTATAGGACTGCCATGAAACTCCGAAGACTGCAGAAGGCCCTTTGCTTGGATCTCTTGAGCCTGTCAGCT
GCATGTGATGCCTTGGACCAGCACAACCTCAAGCAAAATGACCAGCCCATGGATATCCTGCAGATTATTAATTGT
TTGACCACTATTTATGACCGCCTGGAGCAAGAGCACAACAATTTGGTCAACGTCCCTCTCTGCGTGGATATGTGT
CTGAACTGGCTGCTGAATGTTTATGATACGGGACGAACAGGGAGGATCCGTGTCCTGTCTTTTAAAACTGGCATC
ATTTCCCTGTGTAAAGCACATTTGGAAGACAAGTACAGATACCTTTTCAAGCAAGTGGCAAGTTCAACAGGATTT
TGTGACCAGCGCAGGCTGGGCCTCCTTCTGCATGATTCTATCCAAATTCCAAGACAGTTGGGTGAAGTTGCATCC
TTTGGGGGCAGTAACATTGAGCCAAGTGTCCGGAGCTGCTTCCAATTTGCTAATAATAAGCCAGAGATCGAAGCG
GCCCTCTTCCTAGACTGGATGAGACTGGAACCCCAGTCCATGGTGTGGCTGCCCGTCCTGCACAGAGTGGCTGCT
GCAGAAACTGCCAAGCATCAGGCCAAATGTAACATCTGCAAAGAGTGTCCAATCATTGGATTCAGGTACAGGAGT
CTAAAGCACTTTAATTATGACATCTGCCAAAGCTGCTTTTTTTCTGGTCGAGTTGCAAAAGGCCATAAAATGCAC
TATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGG
GACAACATGGAAACTCCCGTTACTCTGATCAACTTCTGGCCAGTAGATTCTGCGCCTGCCTCGTCCCCTCAGCTT
TCACACGATGATACTCATTCACGCATTGAACATTATGCTAGCAGGCTAGCAGAAATGGAAAACAGCAATGGATCT
TATCTAAATGATAGCATCTCTCCTAATGAGAGCATAGATGATGAACATTTGTTAATCCAGCATTACTGCCAAAGT
TTGAACCAGGACTCCCCCCTGAGCCAGCCTCGTAGTCCTGCCCAGATCTTGATTTCCTTAGAGAGTGAGGAAAGA
GGGGAGCTAGAGAGAATCCTAGCAGATCTTGAGGAAGAAAACAGGAATCTGCAAGCAGAATATGACCGTCTAAAG
CAGCAGCACGAACATAAAGGCCTGTCCCCACTGCCGTCCCCTCCTGAAATGATGCCCACCTCTCCCCAGAGTCCC
Nterm-R2_R16-R17_R24 to 1st coiled coil and proline rich region that follows:
(SEQ ID NO: 25)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACACTGTCCGTGAAGAAACGATGATGGTGATGACTGAAGACATGCCTTTGGAAATT
TCTTATGTGCCTTCTACTTATTTGACTGAAATCACTCATGTCTCACAAGCCCTATTAGAAGTGGAACAACTTCTC
AATGCTCCTGACCTCTGTGCTAAGGACTTTGAAGATCTCTTTAAGCAAGAGGAGTCTCTGAAGAATATAAAAGAT
AGTCTACAACAAAGCTCAGGTCGGATTGACATTATTCATAGCAAGAAGACAGCAGCATTGCAAAGTGCAACGCCT
GTGGAAAGGGTGAAGCTACAGGAAGCTCTCTCCCAGCTTGATTTCCAATGGGAAAAAGTTAACAAAATGTACAAG
GACCGACAAGGGCGATTTGACAGATCTGTTGAGAAATGGCGGCGTTTTCATTATGATATAAAGATATTTAATCAG
TGGCTAACAGAAGCTGAACAGTTTCTCAGAAAGACACAAATTCCTGAGAATTGGGAACATGCTAAATACAAATGG
TATCTTAAGGAACTCCAGGATGGCATTGGGCAGCGGCAAACTGTTGTCAGAACATTGAATGCAACTGGGGAAGAA
ATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGTGGCAGAGAAAAATAGATGAGACC
CTTGAAAGACTCCGGGAACTTCAAGAGGCCACGGATGAGCTGGACCTCAAGCTGCGCCAAGCTGAGGTGATCAAG
GGATCCTGGCAGCCCGTGGGCGATCTCCTCATTGACTCTCTCCAAGATCACCTCGAGAAAGTCAAGGCACTTCGA
GGAGAAATTGCGCCTCTGAAAGAGAACGTGAGCCACGTCAATGACCTTGCTCGCCAGCTTACCACTTTGGGCATT
CAGCTCTCACCGTATAACCTCAGCACTCTGGAAGACCTGAACACCAGATGGAAGCTTCTGCAGGTGGCCGTCGAG
GACCGAGTCAGGCAGCTGCATGAAGCCCACAGGGACTTTGGTCCAGCATCTCAGCACTTTCTTTCCACGTCTGTC
CAGGGTCCCTGGGAGAGAGCCATCTCGCCAAACAAAGTGCCCTACTATATCAACCACGAGACTCAAACAACTTGC
TGGGACCATCCCAAAATGACAGAGCTCTACCAGTCTTTAGCTGACCTGAATAATGTCAGATTCTCAGCTTATAGG
ACTGCCATGAAACTCCGAAGACTGCAGAAGGCCCTTTGCTTGGATCTCTTGAGCCTGTCAGCTGCATGTGATGCC
TTGGACCAGCACAACCTCAAGCAAAATGACCAGCCCATGGATATCCTGCAGATTATTAATTGTTTGACCACTATT
TATGACCGCCTGGAGCAAGAGCACAACAATTTGGTCAACGTCCCTCTCTGCGTGGATATGTGTCTGAACTGGCTG
CTGAATGTTTATGATACGGGACGAACAGGGAGGATCCGTGTCCTGTCTTTTAAAACTGGCATCATTTCCCTGTGT
AAAGCACATTTGGAAGACAAGTACAGATACCTTTTCAAGCAAGTGGCAAGTTCAACAGGATTTTGTGACCAGCGC
AGGCTGGGCCTCCTTCTGCATGATTCTATCCAAATTCCAAGACAGTTGGGTGAAGTTGCATCCTTTGGGGGCAGT
AACATTGAGCCAAGTGTCCGGAGCTGCTTCCAATTTGCTAATAATAAGCCAGAGATCGAAGCGGCCCTCTTCCTA
GACTGGATGAGACTGGAACCCCAGTCCATGGTGTGGCTGCCCGTCCTGCACAGAGTGGCTGCTGCAGAAACTGCC
AAGCATCAGGCCAAATGTAACATCTGCAAAGAGTGTCCAATCATTGGATTCAGGTACAGGAGTCTAAAGCACTTT
AATTATGACATCTGCCAAAGCTGCTTTTTTTCTGGTCGAGTTGCAAAAGGCCATAAAATGCACTATCCCATGGTG
GAATATTGCACTCCGACTACATCAGGAGAAGATGTTCGAGACTTTGCCAAGGTACTAAAAAACAAATTTCGAACC
AAAAGGTATTTTGCGAAGCATCCCCGAATGGGCTACCTGCCAGTGCAGACTGTCTTAGAGGGGGACAACATGGAA
ACTCCCGTTACTCTGATCAACTTCTGGCCAGTAGATTCTGCGCCTGCCTCGTCCCCTCAGCTTTCACACGATGAT
ACTCATTCACGCATTGAACATTATGCTAGCAGGCTAGCAGAAATGGAAAACAGCAATGGATCTTATCTAAATGAT
AGCATCTCTCCTAATGAGAGCATAGATGATGAACATTTGTTAATCCAGCATTACTGCCAAAGTTTGAACCAGGAC
TCCCCCCTGAGCCAGCCTCGTAGTCCTGCCCAGATCTTGATTTCCTTAGAGAGTGAGGAAAGAGGGGAGCTAGAG
AGAATCCTAGCAGATCTTGAGGAAGAAAACAGGAATCTGCAAGCAGAATATGACCGTCTAAAGCAGCAGCACGAA
CATAAAGGCCTGTCCCCACTGCCGTCCCCTCCTGAAATGATGCCCACCTCTCCCCAGAGTCCC
Nterm-R2_R23_R24 to end of dystrophin:
(SEQ ID NO: 26)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACCTTTCTCTGCAGGAACTTCTGGTGTGGCTACAGCTGAAAGATGATGAATTAAGC
CGGCAGGCACCTATTGGAGGCGACTTTCCAGCAGTTCAGAAGCAGAACGATGTACATAGGGCCTTCAAGAGGGAA
TTGAAAACTAAAGAACCTGTAATCATGAGTACTCTTGAGACTGTACGAATATTTCTGACAGAGCAGCCTTTGGAA
GGACTAGAGAAACTCTACCAGGAGCCCAGAGAGCTGCCTCCTGAGGAGAGAGCCCAGAATGTCACTCGGCTTCTA
CGAAAGCAGGCTGAGGAGGTCAATACTGAGTGGGAAAAATTGAACCTGCACTCCGCTGACTGGCAGAGAAAAATA
GATGAGACCCTTGAAAGACTCCGGGAACTTCAAGAGGCCACGGATGAGCTGGACCTCAAGCTGCGCCAAGCTGAG
GTGATCAAGGGATCCTGGCAGCCCGTGGGCGATCTCCTCATTGACTCTCTCCAAGATCACCTCGAGAAAGTCAAG
GCACTTCGAGGAGAAATTGCGCCTCTGAAAGAGAACGTGAGCCACGTCAATGACCTTGCTCGCCAGCTTACCACT
TTGGGCATTCAGCTCTCACCGTATAACCTCAGCACTCTGGAAGACCTGAACACCAGATGGAAGCTTCTGCAGGTG
GCCGTCGAGGACCGAGTCAGGCAGCTGCATGAAGCCCACAGGGACTTTGGTCCAGCATCTCAGCACTTTCTTTCC
ACGTCTGTCCAGGGTCCCTGGGAGAGAGCCATCTCGCCAAACAAAGTGCCCTACTATATCAACCACGAGACTCAA
ACAACTTGCTGGGACCATCCCAAAATGACAGAGCTCTACCAGTCTTTAGCTGACCTGAATAATGTCAGATTCTCA
GCTTATAGGACTGCCATGAAACTCCGAAGACTGCAGAAGGCCCTTTGCTTGGATCTCTTGAGCCTGTCAGCTGCA
TGTGATGCCTTGGACCAGCACAACCTCAAGCAAAATGACCAGCCCATGGATATCCTGCAGATTATTAATTGTTTG
ACCACTATTTATGACCGCCTGGAGCAAGAGCACAACAATTTGGTCAACGTCCCTCTCTGCGTGGATATGTGTCTG
AACTGGCTGCTGAATGTTTATGATACGGGACGAACAGGGAGGATCCGTGTCCTGTCTTTTAAAACTGGCATCATT
TCCCTGTGTAAAGCACATTTGGAAGACAAGTACAGATACCTTTTCAAGCAAGTGGCAAGTTCAACAGGATTTTGT
GACCAGCGCAGGCTGGGCCTCCTTCTGCATGATTCTATCCAAATTCCAAGACAGTTGGGTGAAGTTGCATCCTTT
GGGGGCAGTAACATTGAGCCAAGTGTCCGGAGCTGCTTCCAATTTGCTAATAATAAGCCAGAGATCGAAGCGGCC
CTCTTCCTAGACTGGATGAGACTGGAACCCCAGTCCATGGTGTGGCTGCCCGTCCTGCACAGAGTGGCTGCTGCA
GAAACTGCCAAGCATCAGGCCAAATGTAACATCTGCAAAGAGTGTCCAATCATTGGATTCAGGTACAGGAGTCTA
AAGCACTTTAATTATGACATCTGCCAAAGCTGCTTTTTTTCTGGTCGAGTTGCAAAAGGCCATAAAATGCACTAT
CCCATGGTGGAATATTGCACTCCGACTACATCAGGAGAAGATGTTCGAGACTTTGCCAAGGTACTAAAAAACAAA
TTTCGAACCAAAAGGTATTTTGCGAAGCATCCCCGAATGGGCTACCTGCCAGTGCAGACTGTCTTAGAGGGGGAC
AACATGGAAACTCCC
GTTACTCTGATCAACTTCTGGCCAGTAGATTCTGCGCCTGCCTCGTCCCCTCAGCTTTCACACGATGATACTCAT
TCACGCATTGAACATTATGCTAGCAGGCTAGCAGAAATGGAAAACAGCAATGGATCTTATCTAAATGATAGCATC
TCTCCTAATGAGAGCATAGATGATGAACATTTGTTAATCCAGCATTACTGCCAAAGTTTGAACCAGGACTCCCCC
CTGAGCCAGCCTCGTAGTCCTGCCCAGATCTTGATTTCCTTAGAGAGTGAGGAAAGAGGGGAGCTAGAGAGAATC
CTAGCAGATCTTGAGGAAGAAAACAGGAATCTGCAAGCAGAATATGACCGTCTAAAGCAGCAGCACGAACATAAA
GGCCTGTCCCCACTGCCGTCCCCTCCTGAAATGATGCCCACCTCTCCCCAGAGTCCCCGGGATGCTGAGCTCATT
GCTGAGGCCAAGCTACTGCGTCAACACAAAGGCCGCCTGGAAGCCAGGATGCAAATCCTGGAAGACCACAATAAA
CAGCTGGAGTCACAGTTACACAGGCTAAGGCAGCTGCTGGAGCAACCCCAGGCAGAGGCCAAAGTGAATGGCACA
ACGGTGTCCTCTCCTTCTACCTCTCTACAGAGGTCCGACAGCAGTCAGCCTATGCTGCTCCGAGTGGTTGGCAGT
CAAACTTCGGACTCCATGGGTGAGGAAGATCTTCTCAGTCCTCCCCAGGACACAAGCACAGGGTTAGAGGAGGTG
ATGGAGCAACTCAACAACTCCTTCCCTAGTTCAAGAGGAAGAAATACCCCTGGAAAGCCAATGAGAGAGGACACA
ATG
Nterm-R2_R23_R24 to end of dystrophin:
(SEQ ID NO: 133)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACGCCAGTTCTGACCAGTGGAAGCGTCTGCACCTTTCTCTGCAGGAACTTCTGGTGTGG
CTACAGCTGAAAGATGATGAATTAAGCCGGCAGGCACCTATTGGAGGCGACTTTCCAGCAGTTCAGAAGCAGAAC
GATGTACATAGGGCCTTCAAGAGGGAATTGAAAACTAAAGAACCTGTAATCATGAGTACTCTTGAGACTGTACGA
ATATTTCTGACAGAGCAGCCTTTGGAAGGACTAGAGAAACTCTACCAGGAGCCCAGAGAGCTGCCTCCTGAGGAG
AGAGCCCAGAATGTCACTCGGCTTCTACGAAAGCAGGCTGAGGAGGTCAATACTGAGTGGGAAAAATTGAACCTG
CACTCCGCTGACTGGCAGAGAAAAATAGATGAGACCCTTGAAAGACTCCGGGAACTTCAAGAGGCCACGGATGAG
CTGGACCTCAAGCTGCGCCAAGCTGAGGTGATCAAGGGATCCTGGCAGCCCGTGGGCGATCTCCTCATTGACTCT
CTCCAAGATCACCTCGAGAAAGTCAAGGCACTTCGAGGAGAAATTGCGCCTCTGAAAGAGAACGTGAGCCACGTC
AATGACCTTGCTCGCCAGCTTACCACTTTGGGCATTCAGCTCTCACCGTATAACCTCAGCACTCTGGAAGACCTG
AACACCAGATGGAAGCTTCTGCAGGTGGCCGTCGAGGACCGAGTCAGGCAGCTGCATGAAGCCCACAGGGACTTT
GGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTA
GCTGACCTGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAACAATTTGGTCAAC
GTCCCTCTCTGCGTGGATATGTGTCTGAACTGGCTGCTGAATGTTTATGATACGGGACGAACAGGGAGGATCCGT
GTCCTGTCTTTTAAAACTGGCATCATTTCCCTGTGTAAAGCACATTTGGAAGACAAGTACAGATACCTTTTCAAG
CAAGTGGCAAGTTCAACAGGATTTTGTGACCAGCGCAGGCTGGGCCTCCTTCTGCATGATTCTATCCAAATTCCA
AGACAGTTGGGTGAAGTTGCATCCTTTGGGGGCAGTAACATTGAGCCAAGTGTCCGGAGCTGCTTCCAATTTGCT
AATAATAAGCCAGAGATCGAAGCGGCCCTCTTCCTAGACTGGATGAGACTGGAACCCCAGTCCATGGTGTGGCTG
CCCGTCCTGCACAGAGTGGCTGCTGCAGAAACTGCCAAGCATCAGGCCAAATGTAACATCTGCAAAGAGTGTCCA
ATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGA
GACTTTGCCAAGGTACTAAAAAACAAATTTCGAACCAAAAGGTATTTTGCGAAGCATCCCCGAATGGGCTACCTG
CCAGTGCAGACTGTCTTAGAGGGGGACAACATGGAAACTCCCGTTACTCTGATCAACTTCTGGCCAGTAGATTCT
GCGCCTGCCTCGTCCCCTCAGCTTTCACACGATGATACTCATTCACGCATTGAACATTATGCTAGCAGGCTAGCA
GAAATGGAAAACAGCAATGGATCTTATCTAAATGATAGCATCTCTCCTAATGAGAGCATAGATGATGAACATTTG
TTAATCCAGCATTACTGCCAAAGTTTGAACCAGGACTCCCCCCTGAGCCAGCCTCGTAGTCCTGCCCAGATCTTG
ATTTCCTTAGAGAGTGAGGAAAGAGGGGAGCTAGAGAGAATCCTAGCAGATCTTGAGGAAGAAAACAGGAATCTG
CAAGCAGAATATGACCGTCTAAAGCAGCAGCACGAACATAAAGGCCTGTCCCCACTGCCGTCCCCTCCTGAAATG
ATGCCCACCTCTCCCCAGAGTCCCCGGGATGCTGAGCTCATTGCTGAGGCCAAGCTACTGCGTCAACACAAAGGC
CGCCTGGAAGCCAGGATGCAAATCCTGGAAGACCACAATAAACAGCTGGAGTCACAGTTACACAGGCTAAGGCAG
CTGCTGGAGCAACCCCAGGCAGAGGCCAAAGTGAATGGCACAACGGTGTCCTCTCCTTCTACCTCTCTACAGAGG
TCCGACAGCAGTCAGCCTATGCTGCTCCGAGTGGTTGGCAGTCAAACTTCGGACTCCATGGGTGAGGAAGATCTT
CTCAGTCCTCCCCAGGACACAAGCACAGGGTTAGAGGAGGTGATGGAGCAACTCAACAACTCCTTCCCTAGTTCA
AGAGGAAGAAATACCCCTGGAAAGCCAATGAGAGAGGACACAATG
Nterm-R2_R16-R17_H4 to end:
(SEQ ID NO: 27)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACACTGTCCGTGAAGAAACGATGATGGTGATGACTGAAGACATGCCTTTGGAAATT
TCTTATGTGCCTTCTACTTATTTGACTGAAATCACTCATGTCTCACAAGCCCTATTAGAAGTGGAACAACTTCTC
AATGCTCCTGACCTCTGTGCTAAGGACTTTGAAGATCTCTTTAAGCAAGAGGAGTCTCTGAAGAATATAAAAGAT
AGTCTACAACAAAGCTCAGGTCGGATTGACATTATTCATAGCAAGAAGACAGCAGCATTGCAAAGTGCAACGCCT
GTGGAAAGGGTGAAGCTACAGGAAGCTCTCTCCCAGCTTGATTTCCAATGGGAAAAAGTTAACAAAATGTACAAG
GACCGACAAGGGCGATTTGACAGATCTGTTGAGAAATGGCGGCGTTTTCATTATGATATAAAGATATTTAATCAG
TGGCTAACAGAAGCTGAACAGTTTCTCAGAAAGACACAAATTCCTGAGAATTGGGAACATGCTAAATACAAATGG
TATCTTAAGGAACTCCAGGATGGCATTGGGCAGCGGCAAACTGTTGTCAGAACATTGAATGCAACTGGGGAAGAA
ATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGGGTCCAGCATCTCAGCACTTTCTT
TCCACGTCTGTCCAGGGTCCCTGGGAGAGAGCCATCTCGCCAAACAAAGTGCCCTACTATATCAACCACGAGACT
CAAACAACTTGCTGGGACCATCCCAAAATGACAGAGCTCTACCAGTCTTTAGCTGACCTGAATAATGTCAGATTC
TCAGCTTATAGGACTGCCATGAAACTCCGAAGACTGCAGAAGGCCCTTTGCTTGGATCTCTTGAGCCTGTCAGCT
GCATGTGATGCCTTGGACCAGCACAACCTCAAGCAAAATGACCAGCCCATGGATATCCTGCAGATTATTAATTGT
TTGACCACTATTTATGACCGCCTGGAGCAAGAGCACAACAATTTGGTCAACGTCCCTCTCTGCGTGGATATGTGT
CTGAACTGGCTGCTGAATGTTTATGATACGGGACGAACAGGGAGGATCCGTGTCCTGTCTTTTAAAACTGGCATC
ATTTCCCTGTGTAAAGCACATTTGGAAGACAAGTACAGATACCTTTTCAAGCAAGTGGCAAGTTCAACAGGATTT
TGTGACCAGCGCAGGCTGGGCCTCCTTCTGCATGATTCTATCCAAATTCCAAGACAGTTGGGTGAAGTTGCATCC
TTTGGGGGCAGTAACATTGAGCCAAGTGTCCGGAGCTGCTTCCAATTTGCTAATAATAAGCCAGAGATCGAAGCG
GCCCTCTTCCTAGACTGGATGAGACTGGAACCCCAGTCCATGGTGTGGCTGCCCGTCCTGCACAGAGTGGCTGCT
GCAGAAACTGCCAAGCATCAGGCCAAATGTAACATCTGCAAAGAGTGTCCAATCATTGGATTCAGGTACAGGAGT
CTAAAGCACTTTAATTATGACATCTGCCAAAGCTGCTTTTTTTCTGGTCGAGTTGCAAAAGGCCATAAAATGCAC
TATCCCATGGTGGAATATTGCACTCCGACTACATCAGGAGAAGATGTTCGAGACTTTGCCAAGGTACTAAAAAAC
AAATTTCGAACCAAAAGGTATTTTGCGAAGCATCCCCGAATGGGCTACCTGCCAGTGCAGACTGTCTTAGAGGGG
GACAACATGGAAACTCCCGTTACTCTGATCAACTTCTGGCCAGTAGATTCTGCGCCTGCCTCGTCCCCTCAGCTT
TCACACGATGATACTCATTCACGCATTGAACATTATGCTAGCAGGCTAGCAGAAATGGAAAACAGCAATGGATCT
TATCTAAATGATAGCATCTCTCCTAATGAGAGCATAGATGATGAACATTTGTTAATCCAGCATTACTGCCAAAGT
TTGAACCAGGACTCCCCCCTGAGCCAGCCTCGTAGTCCTGCCCAGATCTTGATTTCCTTAGAGAGTGAGGAAAGA
GGGGAGCTAGAGAGAATCCTAGCAGATCTTGAGGAAGAAAACAGGAATCTGCAAGCAGAATATGACCGTCTAAAG
CAGCAGCACGAACATAAAGGCCTGTCCCCACTGCCGTCCCCTCCTGAAATGATGCCCACCTCTCCCCAGAGTCCC
CGGGATGCTGAGCTCATTGCTGAGGCCAAGCTACTGCGTCAACACAAAGGCCGCCTGGAAGCCAGGATGCAAATC
CTGGAAGACCACAATAAACAGCTGGAGTCACAGTTACACAGGCTAAGGCAGCTGCTGGAGCAACCCCAGGCAGAG
GCCAAAGTGAATGGCACAACGGTGTCCTCTCCTTCTACCTCTCTACAGAGGTCCGACAGCAGTCAGCCTATGCTG
CTCCGAGTGGTTGGCAGTCAAACTTCGGACTCCATGGGTGAGGAAGATCTTCTCAGTCCTCCCCAGGACACAAGC
ACAGGGTTAGAGGAGGTGATGGAGCAACTCAACAACTCCTTCCCTAGTTCAAGAGGAAGAAATACCCCTGGAAAG
CCAATGAGAGAGGACACAATG
Nterm-R1_R16-R17_R24 to end:
(SEQ ID NO: 28)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCACACTGTCCGTGAAGAAACGATGATGGTGATGACTGAAGACATGCCTTTGGAAATTTCTTATGTGCCTTCT
ACTTATTTGACTGAAATCACTCATGTCTCACAAGCCCTATTAGAAGTGGAACAACTTCTCAATGCTCCTGACCTC
TGTGCTAAGGACTTTGAAGATCTCTTTAAGCAAGAGGAGTCTCTGAAGAATATAAAAGATAGTCTACAACAAAGC
TCAGGTCGGATTGACATTATTCATAGCAAGAAGACAGCAGCATTGCAAAGTGCAACGCCTGTGGAAAGGGTGAAG
CTACAGGAAGCTCTCTCCCAGCTTGATTTCCAATGGGAAAAAGTTAACAAAATGTACAAGGACCGACAAGGGCGA
TTTGACAGATCTGTTGAGAAATGGCGGCGTTTTCATTATGATATAAAGATATTTAATCAGTGGCTAACAGAAGCT
GAACAGTTTCTCAGAAAGACACAAATTCCTGAGAATTGGGAACATGCTAAATACAAATGGTATCTTAAGGAACTC
CAGGATGGCATTGGGCAGCGGCAAACTGTTGTCAGAACATTGAATGCAACTGGGGAAGAAATAATTCAGCAATCC
TCAAAAACAGATGCCAGTATTCTACAGGAAAAATTGGGAAGCCTGAATCTGCGGTGGCAGGAGGTCTGCAAACAG
CTGTCAGACAGAAAAAAGAGGCTAGAAGAACAAAAGTGGCAGAGAAAAATAGATGAGACCCTTGAAAGACTCCGG
GAACTTCAAGAGGCCACGGATGAGCTGGACCTCAAGCTGCGCCAAGCTGAGGTGATCAAGGGATCCTGGCAGCCC
GTGGGCGATCTCCTCATTGACTCTCTCCAAGATCACCTCGAGAAAGTCAAGGCACTTCGAGGAGAAATTGCGCCT
CTGAAAGAGAACGTGAGCCACGTCAATGACCTTGCTCGCCAGCTTACCACTTTGGGCATTCAGCTCTCACCGTAT
AACCTCAGCACTCTGGAAGACCTGAACACCAGATGGAAGCTTCTGCAGGTGGCCGTCGAGGACCGAGTCAGGCAG
CTGCATGAAGCCCACAGGGACTTTGGTCCAGCATCTCAGCACTTTCTTTCCACGTCTGTCCAGGGTCCCTGGGAG
AGAGCCATCTCGCCAAACAAAGTGCCCTACTATATCAACCACGAGACTCAAACAACTTGCTGGGACCATCCCAAA
ATGACAGAGCTCTACCAGTCTTTAGCTGACCTGAATAATGTCAGATTCTCAGCTTATAGGACTGCCATGAAACTC
CGAAGACTGCAGAAGGCCCTTTGCTTGGATCTCTTGAGCCTGTCAGCTGCATGTGATGCCTTGGACCAGCACAAC
CTCAAGCAAAATGACCAGCCCATGGATATCCTGCAGATTATTAATTGTTTGACCACTATTTATGACCGCCTGGAG
CAAGAGCACAACAATTTGGTCAACGTCCCTCTCTGCGTGGATATGTGTCTGAACTGGCTGCTGAATGTTTATGAT
ACGGGACGAACAGGGAGGATCCGTGTCCTGTCTTTTAAAACTGGCATCATTTCCCTGTGTAAAGCACATTTGGAA
GACAAGTACAGATACCTTTTCAAGCAAGTGGCAAGTTCAACAGGATTTTGTGACCAGCGCAGGCTGGGCCTCCTT
CTGCATGATTCTATCCAAATTCCAAGACAGTTGGGTGAAGTTGCATCCTTTGGGGGCAGTAACATTGAGCCAAGT
GTCCGGAGCTGCTTCCAATTTGCTAATAATAAGCCAGAGATCGAAGCGGCCCTCTTCCTAGACTGGATGAGACTG
GAACCCCAGTCCATGGTGTGGCTGCCCGTCCTGCACAGAGTGGCTGCTGCAGAAACTGCCAAGCATCAGGCCAAA
TGTAACATCTGCAAAGAGTGTCCAATCATTGGATTCAGGTACAGGAGTCTAAAGCACTTTAATTATGACATCTGC
CAAAGCTGCTTTTTTTCTGGTCGAGTTGCAAAAGGCCATAAAATGCACTATCCCATGGTGGAATATTGCACTCCG
ACTACATCAGGAGAAGATGTTCGAGACTTTGCCAAGGTACTAAAAAACAAATTTCGAACCAAAAGGTATTTTGCG
AAGCATCCCCGAATGGGCTACCTGCCAGTGCAGACTGTCTTAGAGGGGGACAACATGGAAACTCCCGTTACTCTG
ATCAACTTCTGGCCAGTAGATTCTGCGCCTGCCTCGTCCCCTCAGCTTTCACACGATGATACTCATTCACGCATT
GAACATTATGCTAGCAGGCTAGCAGAAATGGAAAACAGCAATGGATCTTATCTAAATGATAGCATCTCTCCTAAT
GAGAGCATAGATGATGAACATTTGTTAATCCAGCATTACTGCCAAAGTTTGAACCAGGACTCCCCCCTGAGCCAG
CCTCGTAGTCCTGCCCAGATCTTGATTTCCTTAGAGAGTGAGGAAAGAGGGGAGCTAGAGAGAATCCTAGCAGAT
CTTGAGGAAGAAAACAGGAATCTGCAAGCAGAATATGACCGTCTAAAGCAGCAGCACGAACATAAAGGCCTGTCC
CCACTGCCGTCCCCTCCTGAAATGATGCCCACCTCTCCCCAGAGTCCCCGGGATGCTGAGCTCATTGCTGAGGCC
AAGCTACTGCGTCAACACAAAGGCCGCCTGGAAGCCAGGATGCAAATCCTGGAAGACCACAATAAACAGCTGGAG
TCACAGTTACACAGGCTAAGGCAGCTGCTGGAGCAACCCCAGGCAGAGGCCAAAGTGAATGGCACAACGGTGTCC
TCTCCTTCTACCTCTCTACAGAGGTCCGACAGCAGTCAGCCTATGCTGCTCCGAGTGGTTGGCAGTCAAACTTCG
GACTCCATGGGTGAGGAAGATCTTCTCAGTCCTCCCCAGGACACAAGCACAGGGTTAGAGGAGGTGATGGAGCAA
CTCAACAACTCCTTCCCTAGTTCAAGAGGAAGAAATACCCCTGGAAAGCCAATGAGAGAGGACACAATG
Nterm-R2_R16-R17_R24 to end:
(SEQ ID NO: 29)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGITGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACACTGTCCGTGAAGAAACGATGATGGTGATGACTGAAGACATGCCTTTGGAAATT
TCTTATGTGCCTTCTACTTATTTGACTGAAATCACTCATGTCTCACAAGCCCTATTAGAAGTGGAACAACTTCTC
AATGCTCCTGACCTCTGTGCTAAGGACTTTGAAGATCTCTTTAAGCAAGAGGAGTCTCTGAAGAATATAAAAGAT
AGTCTACAACAAAGCTCAGGTCGGATTGACATTATTCATAGCAAGAAGACAGCAGCATTGCAAAGTGCAACGCCT
GTGGAAAGGGTGAAGCTACAGGAAGCTCTCTCCCAGCTTGATTTCCAATGGGAAAAAGTTAACAAAATGTACAAG
GACCGACAAGGGCGATTTGACAGATCTGTTGAGAAATGGCGGCGTTTTCATTATGATATAAAGATATTTAATCAG
TGGCTAACAGAAGCTGAACAGTTTCTCAGAAAGACACAAATTCCTGAGAATTGGGAACATGCTAAATACAAATGG
TATCTTAAGGAACTCCAGGATGGCATTGGGCAGCGGCAAACTGTTGTCAGAACATTGAATGCAACTGGGGAAGAA
ATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGTGGCAGAGAAAAATAGATGAGACC
CTTGAAAGACTCCGGGAACTTCAAGAGGCCACGGATGAGCTGGACCTCAAGCTGCGCCAAGCTGAGGTGATCAAG
GGATCCTGGCAGCCCGTGGGCGATCTCCTCATTGACTCTCTCCAAGATCACCTCGAGAAAGTCAAGGCACTTCGA
GGAGAAATTGCGCCTCTGAAAGAGAACGTGAGCCACGTCAATGACCTTGCTCGCCAGCTTACCACTTTGGGCATT
CAGCTCTCACCGTATAACCTCAGCACTCTGGAAGACCTGAACACCAGATGGAAGCTTCTGCAGGTGGCCGTCGAG
GACCGAGTCAGGCAGCTGCATGAAGCCCACAGGGACTTTGGTCCAGCATCTCAGCACTTTCTTTCCACGTCTGTC
CAGGGTCCCTGGGAGAGAGCCATCTCGCCAAACAAAGTGCCCTACTATATCAACCACGAGACTCAAACAACTTGC
TGGGACCATCCCAAAATGACAGAGCTCTACCAGTCTTTAGCTGACCTGAATAATGTCAGATTCTCAGCTTATAGG
ACTGCCATGAAACTCCGAAGACTGCAGAAGGCCCTTTGCTTGGATCTCTTGAGCCTGTCAGCTGCATGTGATGCC
TTGGACCAGCACAACCTCAAGCAAAATGACCAGCCCATGGATATCCTGCAGATTATTAATTGTTTGACCACTATT
TATGACCGCCTGGAGCAAGAGCACAACAATTTGGTCAACGTCCCTCTCTGCGTGGATATGTGTCTGAACTGGCTG
CTGAATGTTTATGATACGGGACGAACAGGGAGGATCCGTGTCCTGTCTTTTAAAACTGGCATCATTTCCCTGTGT
AAAGCACATTTGGAAGACAAGTACAGATACCTTTTCAAGCAAGTGGCAAGTTCAACAGGATTTTGTGACCAGCGC
AGGCTGGGCCTCCTTCTGCATGATTCTATCCAAATTCCAAGACAGTTGGGTGAAGTTGCATCCTTTGGGGGCAGT
AACATTGAGCCAAGTGTCCGGAGCTGCTTCCAATTTGCTAATAATAAGCCAGAGATCGAAGCGGCCCTCTTCCTA
GACTGGATGAGACTGGAACCCCAGTCCATGGTGTGGCTGCCCGTCCTGCACAGAGTGGCTGCTGCAGAAACTGCC
AAGCATCAGGCCAAATGTAACATCTGCAAAGAGTGTCCAATCATTGGATTCAGGTACAGGAGTCTAAAGCACTTT
AATTATGACATCTGCCAAAGCTGCTTTTTTTCTGGTCGAGTTGCAAAAGGCCATAAAATGCACTATCCCATGGTG
GAATATTGCACTCCGACTACATCAGGAGAAGATGTTCGAGACTTTGCCAAGGTACTAAAAAACAAATTTCGAACC
AAAAGGTATTTTGCGAAGCATCCCCGAATGGGCTACCTGCCAGTGCAGACTGTCTTAGAGGGGGACAACATGGAA
ACTCCCGTTACTCTGATCAACTTCTGGCCAGTAGATTCTGCGCCTGCCTCGTCCCCTCAGCTTTCACACGATGAT
ACTCATTCACGCATTGAACATTATGCTAGCAGGCTAGCAGAAATGGAAAACAGCAATGGATCTTATCTAAATGAT
AGCATCTCTCCTAATGAGAGCATAGATGATGAACATTTGTTAATCCAGCATTACTGCCAAAGTTTGAACCAGGAC
TCCCCCCTGAGCCAGCCTCGTAGTCCTGCCCAGATCTTGATTTCCTTAGAGAGTGAGGAAAGAGGGGAGCTAGAG
AGAATCCTAGCAGATCTTGAGGAAGAAAACAGGAATCTGCAAGCAGAATATGACCGTCTAAAGCAGCAGCACGAA
CATAAAGGCCTGTCCCCACTGCCGTCCCCTCCTGAAATGATGCCCACCTCTCCCCAGAGTCCCCGGGATGCTGAG
CTCATTGCTGAGGCCAAGCTACTGCGTCAACACAAAGGCCGCCTGGAAGCCAGGATGCAAATCCTGGAAGACCAC
AATAAACAGCTGGAGTCACAGTTACACAGGCTAAGGCAGCTGCTGGAGCAACCCCAGGCAGAGGCCAAAGTGAAT
GGCACAACGGTGTCCTCTCCTTCTACCTCTCTACAGAGGTCCGACAGCAGTCAGCCTATGCTGCTCCGAGTGGTT
GGCAGTCAAACTTCGGACTCCATGGGTGAGGAAGATCTTCTCAGTCCTCCCCAGGACACAAGCACAGGGTTAGAG
GAGGTGATGGAGCAACTCAACAACTCCTTCCCTAGTTCAAGAGGAAGAAATACCCCTGGAAAGCCAATGAGAGAG
GACACAATG
Nterm-R2_R16-R17_R24 to end of syntrophin region:
(SEQ ID NO: 30)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GATCTCCAGAATCAGAAACTGAAAGAGTTGAATGACTGGCTAACAAAAACAGAAGAAAGAACAAGGAAAATGGAG
GAAGAGCCTCTTGGACCTGATCTTGAAGACCTAAAACGCCAAGTACAACAACATAAGGTGCTTCAAGAAGATCTA
GAACAAGAACAAGTCAGGGTCAATTCTCTCACTCACATGGTGGTGGTAGTTGATGAATCTAGTGGAGATCACGCA
ACTGCTGCTTTGGAAGAACAACTTAAGGTATTGGGAGATCGATGGGCAAACATCTGTAGATGGACAGAAGACCGC
TGGGTTCTTTTACAAGACCACACTGTCCGTGAAGAAACGATGATGGTGATGACTGAAGACATGCCTTTGGAAATT
TCTTATGTGCCTTCTACTTATTTGACTGAAATCACTCATGTCTCACAAGCCCTATTAGAAGTGGAACAACTTCTC
AATGCTCCTGACCTCTGTGCTAAGGACTTTGAAGATCTCTTTAAGCAAGAGGAGTCTCTGAAGAATATAAAAGAT
AGTCTACAACAAAGCTCAGGTCGGATTGACATTATTCATAGCAAGAAGACAGCAGCATTGCAAAGTGCAACGCCT
GTGGAAAGGGTGAAGCTACAGGAAGCTCTCTCCCAGCTTGATTTCCAATGGGAAAAAGTTAACAAAATGTACAAG
GACCGACAAGGGCGATTTGACAGATCTGTTGAGAAATGGCGGCGTTTTCATTATGATATAAAGATATTTAATCAG
TGGCTAACAGAAGCTGAACAGTTTCTCAGAAAGACACAAATTCCTGAGAATTGGGAACATGCTAAATACAAATGG
TATCTTAAGGAACTCCAGGATGGCATTGGGCAGCGGCAAACTGTTGTCAGAACATTGAATGCAACTGGGGAAGAA
ATAATTCAGCAATCCTCAAAAACAGATGCCAGTATTCTACAGGAAAAATTGGGAAGCCTGAATCTGCGGTGGCAG
GAGGTCTGCAAACAGCTGTCAGACAGAAAAAAGAGGCTAGAAGAACAAAAGTGGCAGAGAAAAATAGATGAGACC
CTTGAAAGACTCCGGGAACTTCAAGAGGCCACGGATGAGCTGGACCTCAAGCTGCGCCAAGCTGAGGTGATCAAG
GGATCCTGGCAGCCCGTGGGCGATCTCCTCATTGACTCTCTCCAAGATCACCTCGAGAAAGTCAAGGCACTTCGA
GGAGAAATTGCGCCTCTGAAAGAGAACGTGAGCCACGTCAATGACCTTGCTCGCCAGCTTACCACTTTGGGCATT
CAGCTCTCACCGTATAACCTCAGCACTCTGGAAGACCTGAACACCAGATGGAAGCTTCTGCAGGTGGCCGTCGAG
GACCGAGTCAGGCAGCTGCATGAAGCCCACAGGGACTTTGGTCCAGCATCTCAGCACTTTCTTTCCACGTCTGTC
CAGGGTCCCTGGGAGAGAGCCATCTCGCCAAACAAAGTGCCCTACTATATCAACCACGAGACTCAAACAACTTGC
TGGGACCATCCCAAAATGACAGAGCTCTACCAGTCTTTAGCTGACCTGAATAATGTCAGATTCTCAGCTTATAGG
ACTGCCATGAAACTCCGAAGACTGCAGAAGGCCCTTTGCTTGGATCTCTTGAGCCTGTCAGCTGCATGTGATGCC
TTGGACCAGCACAACCTCAAGCAAAATGACCAGCCCATGGATATCCTGCAGATTATTAATTGTTTGACCACTATT
TATGACCGCCTGGAGCAAGAGCACAACAATTTGGTCAACGTCCCTCTCTGCGTGGATATGTGTCTGAACTGGCTG
CTGAATGTTTATGATACGGGACGAACAGGGAGGATCCGTGTCCTGTCTTTTAAAACTGGCATCATTTCCCTGTGT
AAAGCACATTTGGAAGACAAGTACAGATACCTTTTCAAGCAAGTGGCAAGTTCAACAGGATTTTGTGACCAGCGC
AGGCTGGGCCTCCTTCTGCATGATTCTATCCAAATTCCAAGACAGTTGGGTGAAGTTGCATCCTTTGGGGGCAGT
AACATTGAGCCAAGTGTCCGGAGCTGCTTCCAATTTGCTAATAATAAGCCAGAGATCGAAGCGGCCCTCTTCCTA
GACTGGATGAGACTGGAACCCCAGTCCATGGTGTGGCTGCCCGTCCTGCACAGAGTGGCTGCTGCAGAAACTGCC
AAGCATCAGGCCAAATGTAACATCTGCAAAGAGTGTCCAATCATTGGATTCAGGTACAGGAGTCTAAAGCACTTT
AATTATGACATCTGCCAAAGCTGCTTTTTTTCTGGTCGAGTTGCAAAAGGCCATAAAATGCACTATCCCATGGTG
GAATATTGCACTCCGACTACATCAGGAGAAGATGTTCGAGACTTTGCCAAGGTACTAAAAAACAAATTTCGAACC
AAAAGGTATTTTGCGAAGCATCCCCGAATGGGCTACCTGCCAGTGCAGACTGTCTTAGAGGGGGACAACATGGAA
ACTCCCGTTACTCTGATCAACTTCTGGCCAGTAGATTCTGCGCCTGCCTCGTCCCCTCAGCTTTCACACGATGAT
ACTCATTCACGCATTGAACATTATGCTAGCAGGCTAGCAGAAATGGAAAACAGCAATGGATCTTATCTAAATGAT
AGCATCTCTCCTAATGAGAGCATAGATGATGAACATTTGTTAATCCAGCATTACTGCCAAAGTTTGAACCAGGAC
TCCCCCCTGAGCCAGCCTCGTAGT

In some embodiments, a micro-dystrophin protein as described herein further comprises one or more utrophin protein domains.

Non-limiting examples of codon optimized nucleotide sequences encoding human micro-dystrophin proteins optimized for skeletal muscle are described below.

Nterm-R2_R16-R17_R24 + H4 + trCterm:
(SEQ ID NO: 31)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACACCGTGCGGGAGGAGACCATGATGGTGATGACCGAGGACATGCCCCTGGAGATC
AGCTACGTGCCCAGCACCTACCTGACCGAGATCACCCACGTGAGCCAGGCCCTGCTGGAGGTGGAGCAGCTGCTG
AACGCCCCCGACCTGTGCGCCAAGGACTTCGAGGACCTGTTCAAGCAGGAGGAGAGCCTGAAGAACATCAAGGAC
AGCCTGCAGCAGAGCAGCGGCCGGATCGACATCATCCACAGCAAGAAGACCGCCGCCCTGCAGAGCGCCACCCCC
GTGGAGCGGGTGAAGCTGCAGGAGGCCCTGAGCCAGCTGGACTTCCAGTGGGAGAAGGTGAACAAGATGTACAAG
GACCGGCAGGGCCGGTTCGACCGGAGCGTGGAGAAGTGGCGGCGGTTCCACTACGACATCAAGATCTTCAACCAG
TGGCTGACCGAGGCCGAGCAGTTCCTGCGGAAGACCCAGATCCCCGAGAACTGGGAGCACGCCAAGTACAAGTGG
TACCTGAAGGAGCTGCAGGACGGCATCGGCCAGCGGCAGACCGTGGTGCGGACCCTGAACGCCACCGGCGAGGAG
ATCATCCAGCAGAGCAGCAAGACCGACGCCAGCATCCTGCAGGAGAAGCTGGGCAGCCTGAACCTGCGGTGGCAG
GAGGTGTGCAAGCAGCTGAGCGACCGGAAGAAGCGGCTGGAGGAGCAGAAGTGGCAGCGGAAGATCGACGAGACC
CTGGAGCGGCTGCAGGAGCTGCAGGAGGCCACCGACGAGCTGGACCTGAAGCTGCGGCAGGCCGAGGTGATCAAG
GGCAGCTGGCAGCCCGTGGGCGACCTGCTGATCGACAGCCTGCAGGACCACCTGGAGAAGGTGAAGGCCCTGCGG
GGCGAGATCGCCCCCCTGAAGGAGAACGTGAGCCACGTGAACGACCTGGCCCGGCAGCTGACCACCCTGGGCATC
CAGCTGAGCCCCTACAACCTGAGCACCCTGGAGGACCTGAACACCCGGTGGAAGCTGCTGCAGGTGGCCGTGGAG
GACCGGGTGCGGCAGCTGCACGAGGCCCACCGGGACTTCGGCCCCGCCAGCCAGCACTTCCTGAGCACCAGCGTG
CAGGGCCCCTGGGAGCGGGCCATCAGCCCCAACAAGGTGCCCTACTACATCAACCACGAGACCCAGACCACCTGC
TGGGACCACCCCAAGATGACCGAGCTGTACCAGAGCCTGGCCGACCTGAACAACGTGCGGTTCAGCGCCTACCGG
ACCGCCATGAAGCTGCGGCGGCTGCAGAAGGCCCTGTGCCTGGACCTGCTGAGCCTGAGCGCCGCCTGCGACGCC
CTGGACCAGCACAACCTGAAGCAGAACGACCAGCCCATGGACATCCTGCAGATCATCAACTGCCTGACCACCATC
TACGACCGGCTGGAGCAGGAGCACAACAACCTGGTGAACGTGCCCCTGTGCGTGGACATGTGCCTGAACTGGCTG
CTGAACGTGTACGACACCGGCCGGACCGGCCGGATCCGGGTGCTGAGCTTCAAGACCGGCATCATCAGCCTGTGC
AAGGCCCACCTGGAGGACAAGTACCGGTACCTGTTCAAGCAGGTGGCCAGCAGCACCGGCTTCTGCGACCAGCGG
CGGCTGGGCCTGCTGCTGCACGACAGCATCCAGATCCCCCGGCAGCTGGGCGAGGTGGCCAGCTTCGGCGGCAGC
AACATCGAGCCCAGCGTGCGGAGCTGCTTCCAGTTCGCCAACAACAAGCCCGAGATCGAGGCCGCCCTGTTCCTG
GACTGGATGCGGCTGGAGCCCCAGAGCATGGTGTGGCTGCCCGTGCTGCACCGGGTGGCCGCCGCCGAGACCGCC
AAGCACCAGGCCAAGTGCAACATCTGCAAGGAGTGCCCCATCATCGGCTTCCGGTACCGGAGCCTGAAGCACTTC
AACTACGACATCTGCCAGAGCTGCTTCTTCAGCGGCCGGGTGGCCAAGGGCCACAAGATGCACTACCCCATGGTG
GAGTACTGCACCCCCACCACCAGCGGCGAGGACGTGCGGGACTTCGCCAAGGTGCTGAAGAACAAGTTCCGGACC
AAGCGGTACTTCGCCAAGCACCCCCGGATGGGCTACCTGCCCGTGCAGACCGTGCTGGAGGGCGACAACATGGAG
ACCCCC
Nterm-R2_R16-R17_H4 to end of syntrophin region:
(SEQ ID NO: 32)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACACCGTGCGGGAGGAGACCATGATGGTGATGACCGAGGACATGCCCCTGGAGATC
AGCTACGTGCCCAGCACCTACCTGACCGAGATCACCCACGTGAGCCAGGCCCTGCTGGAGGTGGAGCAGCTGCTG
AACGCCCCCGACCTGTGCGCCAAGGACTTCGAGGACCTGTTCAAGCAGGAGGAGAGCCTGAAGAACATCAAGGAC
AGCCTGCAGCAGAGCAGCGGCCGGATCGACATCATCCACAGCAAGAAGACCGCCGCCCTGCAGAGCGCCACCCCC
GTGGAGCGGGTGAAGCTGCAGGAGGCCCTGAGCCAGCTGGACTTCCAGTGGGAGAAGGTGAACAAGATGTACAAG
GACCGGCAGGGCCGGTTCGACCGGAGCGTGGAGAAGTGGCGGCGGTTCCACTACGACATCAAGATCTTCAACCAG
TGGCTGACCGAGGCCGAGCAGTTCCTGCGGAAGACCCAGATCCCCGAGAACTGGGAGCACGCCAAGTACAAGTGG
TACCTGAAGGAGCTGCAGGACGGCATCGGCCAGCGGCAGACCGTGGTGCGGACCCTGAACGCCACCGGCGAGGAG
ATCATCCAGCAGAGCAGCAAGACCGACGCCAGCATCCTGCAGGAGAAGCTGGGCAGCCTGAACCTGCGGTGGCAG
GAGGTGTGCAAGCAGCTGAGCGACCGGAAGAAGCGGCTGGAGGAGCAGAAGGGCCCCGCCAGCCAGCACTTCCTG
AGCACCAGCGTGCAGGGCCCCTGGGAGCGGGCCATCAGCCCCAACAAGGTGCCCTACTACATCAACCACGAGACC
CAGACCACCTGCTGGGACCACCCCAAGATGACCGAGCTGTACCAGAGCCTGGCCGACCTGAACAACGTGCGGTTC
AGCGCCTACCGGACCGCCATGAAGCTGCGGCGGCTGCAGAAGGCCCTGTGCCTGGACCTGCTGAGCCTGAGCGCC
GCCTGCGACGCCCTGGACCAGCACAACCTGAAGCAGAACGACCAGCCCATGGACATCCTGCAGATCATCAACTGC
CTGACCACCATCTACGACCGGCTGGAGCAGGAGCACAACAACCTGGTGAACGTGCCCCTGTGCGTGGACATGTGC
CTGAACTGGCTGCTGAACGTGTACGACACCGGCCGGACCGGCCGGATCCGGGTGCTGAGCTTCAAGACCGGCATC
ATCAGCCTGTGCAAGGCCCACCTGGAGGACAAGTACCGGTACCTGTTCAAGCAGGTGGCCAGCAGCACCGGCTTC
TGCGACCAGCGGCGGCTGGGCCTGCTGCTGCACGACAGCATCCAGATCCCCCGGCAGCTGGGCGAGGTGGCCAGC
TTCGGCGGCAGCAACATCGAGCCCAGCGTGCGGAGCTGCTTCCAGTTCGCCAACAACAAGCCCGAGATCGAGGCC
GCCCTGTTCCTGGACTGGATGCGGCTGGAGCCCCAGAGCATGGTGTGGCTGCCCGTGCTGCACCGGGTGGCCGCC
GCCGAGACCGCCAAGCACCAGGCCAAGTGCAACATCTGCAAGGAGTGCCCCATCATCGGCTTCCGGTACCGGAGC
CTGAAGCACTTCAACTACGACATCTGCCAGAGCTGCTTCTTCAGCGGCCGGGTGGCCAAGGGCCACAAGATGCAC
TACCCCATGGTGGAGTACTGCACCCCCACCACCAGCGGCGAGGACGTGCGGGACTTCGCCAAGGTGCTGAAGAAC
AAGTTCCGGACCAAGCGGTACTTCGCCAAGCACCCCCGGATGGGCTACCTGCCCGTGCAGACCGTGCTGGAGGGC
GACAACATGGAGACCCCCGTGACCCTGATCAACTTCTGGCCCGTGGACAGCGCCCCCGCCAGCAGCCCCCAGCTG
AGCCACGACGACACCCACAGCCGGATCGAGCACTACGCCAGCCGGCTGGCCGAGATGGAGAACAGCAACGGCAGC
TACCTGAACGACAGCATCAGCCCCAACGAGAGCATCGACGACGAGCACCTGCTGATCCAGCACTACTGCCAGAGC
CTGAACCAGGACAGCCCCCTGAGCCAGCCCCGGAGC
Nterm-R2_R16-R17_H4 to 1st coiled coil and proline rich region that follows:
(SEQ ID NO: 33)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACACCGTGCGGGAGGAGACCATGATGGTGATGACCGAGGACATGCCCCTGGAGATC
AGCTACGTGCCCAGCACCTACCTGACCGAGATCACCCACGTGAGCCAGGCCCTGCTGGAGGTGGAGCAGCTGCTG
AACGCCCCCGACCTGTGCGCCAAGGACTTCGAGGACCTGTTCAAGCAGGAGGAGAGCCTGAAGAACATCAAGGAC
AGCCTGCAGCAGAGCAGCGGCCGGATCGACATCATCCACAGCAAGAAGACCGCCGCCCTGCAGAGCGCCACCCCC
GTGGAGCGGGTGAAGCTGCAGGAGGCCCTGAGCCAGCTGGACTTCCAGTGGGAGAAGGTGAACAAGATGTACAAG
GACCGGCAGGGCCGGTTCGACCGGAGCGTGGAGAAGTGGCGGCGGTTCCACTACGACATCAAGATCTTCAACCAG
TGGCTGACCGAGGCCGAGCAGTTCCTGCGGAAGACCCAGATCCCCGAGAACTGGGAGCACGCCAAGTACAAGTGG
TACCTGAAGGAGCTGCAGGACGGCATCGGCCAGCGGCAGACCGTGGTGCGGACCCTGAACGCCACCGGCGAGGAG
ATCATCCAGCAGAGCAGCAAGACCGACGCCAGCATCCTGCAGGAGAAGCTGGGCAGCCTGAACCTGCGGTGGCAG
GAGGTGTGCAAGCAGCTGAGCGACCGGAAGAAGCGGCTGGAGGAGCAGAAGGGCCCCGCCAGCCAGCACTTCCTG
AGCACCAGCGTGCAGGGCCCCTGGGAGCGGGCCATCAGCCCCAACAAGGTGCCCTACTACATCAACCACGAGACC
CAGACCACCTGCTGGGACCACCCCAAGATGACCGAGCTGTACCAGAGCCTGGCCGACCTGAACAACGTGCGGTTC
AGCGCCTACCGGACCGCCATGAAGCTGCGGCGGCTGCAGAAGGCCCTGTGCCTGGACCTGCTGAGCCTGAGCGCC
GCCTGCGACGCCCTGGACCAGCACAACCTGAAGCAGAACGACCAGCCCATGGACATCCTGCAGATCATCAACTGC
CTGACCACCATCTACGACCGGCTGGAGCAGGAGCACAACAACCTGGTGAACGTGCCCCTGTGCGTGGACATGTGC
CTGAACTGGCTGCTGAACGTGTACGACACCGGCCGGACCGGCCGGATCCGGGTGCTGAGCTTCAAGACCGGCATC
ATCAGCCTGTGCAAGGCCCACCTGGAGGACAAGTACCGGTACCTGTTCAAGCAGGTGGCCAGCAGCACCGGCTTC
TGCGACCAGCGGCGGCTGGGCCTGCTGCTGCACGACAGCATCCAGATCCCCCGGCAGCTGGGCGAGGTGGCCAGC
TTCGGCGGCAGCAACATCGAGCCCAGCGTGCGGAGCTGCTTCCAGTTCGCCAACAACAAGCCCGAGATCGAGGCC
GCCCTGTTCCTGGACTGGATGCGGCTGGAGCCCCAGAGCATGGTGTGGCTGCCCGTGCTGCACCGGGTGGCCGCC
GCCGAGACCGCCAAGCACCAGGCCAAGTGCAACATCTGCAAGGAGTGCCCCATCATCGGCTTCCGGTACCGGAGC
CTGAAGCACTTCAACTACGACATCTGCCAGAGCTGCTTCTTCAGCGGCCGGGTGGCCAAGGGCCACAAGATGCAC
TACCCCATGGTGGAGTACTGCACCCCCACCACCAGCGGCGAGGACGTGCGGGACTTCGCCAAGGTGCTGAAGAAC
AAGTTCCGGACCAAGCGGTACTTCGCCAAGCACCCCCGGATGGGCTACCTGCCCGTGCAGACCGTGCTGGAGGGC
GACAACATGGAGACCCCCGTGACCCTGATCAACTTCTGGCCCGTGGACAGCGCCCCCGCCAGCAGCCCCCAGCTG
AGCCACGACGACACCCACAGCCGGATCGAGCACTACGCCAGCCGGCTGGCCGAGATGGAGAACAGCAACGGCAGC
TACCTGAACGACAGCATCAGCCCCAACGAGAGCATCGACGACGAGCACCTGCTGATCCAGCACTACTGCCAGAGC
CTGAACCAGGACAGCCCCCTGAGCCAGCCCCGGAGCCCCGCCCAGATCCTGATCAGCCTGGAGAGCGAGGAGCGG
GGCGAGCTGGAGCGGATCCTGGCCGACCTGGAGGAGGAGAACCGGAACCTGCAGGCCGAGTACGACCGGCTGAAG
CAGCAGCACGAGCACAAGGGCCTGAGCCCCCTGCCCAGCCCCCCCGAGATGATGCCCACCAGCCCCCAGAGCCCC
Nterm-R2_R16-R17_R24 to 1st coiled coil and proline rich region that follows:
(SEQ ID NO: 34)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACACCGTGCGGGAGGAGACCATGATGGTGATGACCGAGGACATGCCCCTGGAGATC
AGCTACGTGCCCAGCACCTACCTGACCGAGATCACCCACGTGAGCCAGGCCCTGCTGGAGGTGGAGCAGCTGCTG
AACGCCCCCGACCTGTGCGCCAAGGACTTCGAGGACCTGTTCAAGCAGGAGGAGAGCCTGAAGAACATCAAGGAC
AGCCTGCAGCAGAGCAGCGGCCGGATCGACATCATCCACAGCAAGAAGACCGCCGCCCTGCAGAGCGCCACCCCC
GTGGAGCGGGTGAAGCTGCAGGAGGCCCTGAGCCAGCTGGACTTCCAGTGGGAGAAGGTGAACAAGATGTACAAG
GACCGGCAGGGCCGGTTCGACCGGAGCGTGGAGAAGTGGCGGCGGTTCCACTACGACATCAAGATCTTCAACCAG
TGGCTGACCGAGGCCGAGCAGTTCCTGCGGAAGACCCAGATCCCCGAGAACTGGGAGCACGCCAAGTACAAGTGG
TACCTGAAGGAGCTGCAGGACGGCATCGGCCAGCGGCAGACCGTGGTGCGGACCCTGAACGCCACCGGCGAGGAG
ATCATCCAGCAGAGCAGCAAGACCGACGCCAGCATCCTGCAGGAGAAGCTGGGCAGCCTGAACCTGCGGTGGCAG
GAGGTGTGCAAGCAGCTGAGCGACCGGAAGAAGCGGCTGGAGGAGCAGAAGTGGCAGCGGAAGATCGACGAGACC
CTGGAGCGGCTGCAGGAGCTGCAGGAGGCCACCGACGAGCTGGACCTGAAGCTGCGGCAGGCCGAGGTGATCAAG
GGCAGCTGGCAGCCCGTGGGCGACCTGCTGATCGACAGCCTGCAGGACCACCTGGAGAAGGTGAAGGCCCTGCGG
GGCGAGATCGCCCCCCTGAAGGAGAACGTGAGCCACGTGAACGACCTGGCCCGGCAGCTGACCACCCTGGGCATC
CAGCTGAGCCCCTACAACCTGAGCACCCTGGAGGACCTGAACACCCGGTGGAAGCTGCTGCAGGTGGCCGTGGAG
GACCGGGTGCGGCAGCTGCACGAGGCCCACCGGGACTTCGGCCCCGCCAGCCAGCACTTCCTGAGCACCAGCGTG
CAGGGCCCCTGGGAGCGGGCCATCAGCCCCAACAAGGTGCCCTACTACATCAACCACGAGACCCAGACCACCTGC
TGGGACCACCCCAAGATGACCGAGCTGTACCAGAGCCTGGCCGACCTGAACAACGTGCGGTTCAGCGCCTACCGG
ACCGCCATGAAGCTGCGGCGGCTGCAGAAGGCCCTGTGCCTGGACCTGCTGAGCCTGAGCGCCGCCTGCGACGCC
CTGGACCAGCACAACCTGAAGCAGAACGACCAGCCCATGGACATCCTGCAGATCATCAACTGCCTGACCACCATC
TACGACCGGCTGGAGCAGGAGCACAACAACCTGGTGAACGTGCCCCTGTGCGTGGACATGTGCCTGAACTGGCTG
CTGAACGTGTACGACACCGGCCGGACCGGCCGGATCCGGGTGCTGAGCTTCAAGACCGGCATCATCAGCCTGTGC
AAGGCCCACCTGGAGGACAAGTACCGGTACCTGTTCAAGCAGGTGGCCAGCAGCACCGGCTTCTGCGACCAGCGG
CGGCTGGGCCTGCTGCTGCACGACAGCATCCAGATCCCCCGGCAGCTGGGCGAGGTGGCCAGCTTCGGCGGCAGC
AACATCGAGCCCAGCGTGCGGAGCTGCTTCCAGTTCGCCAACAACAAGCCCGAGATCGAGGCCGCCCTGTTCCTG
GACTGGATGCGGCTGGAGCCCCAGAGCATGGTGTGGCTGCCCGTGCTGCACCGGGTGGCCGCCGCCGAGACCGCC
AAGCACCAGGCCAAGTGCAACATCTGCAAGGAGTGCCCCATCATCGGCTTCCGGTACCGGAGCCTGAAGCACTTC
AACTACGACATCTGCCAGAGCTGCTTCTTCAGCGGCCGGGTGGCCAAGGGCCACAAGATGCACTACCCCATGGTG
GAGTACTGCACCCCCACCACCAGCGGCGAGGACGTGCGGGACTTCGCCAAGGTGCTGAAGAACAAGTTCCGGACC
AAGCGGTACTTCGCCAAGCACCCCCGGATGGGCTACCTGCCCGTGCAGACCGTGCTGGAGGGCGACAACATGGAG
ACCCCCGTGACCCTGATCAACTTCTGGCCCGTGGACAGCGCCCCCGCCAGCAGCCCCCAGCTGAGCCACGACGAC
ACCCACAGCCGGATCGAGCACTACGCCAGCCGGCTGGCCGAGATGGAGAACAGCAACGGCAGCTACCTGAACGAC
AGCATCAGCCCCAACGAGAGCATCGACGACGAGCACCTGCTGATCCAGCACTACTGCCAGAGCCTGAACCAGGAC
AGCCCCCTGAGCCAGCCCCGGAGCCCCGCCCAGATCCTGATCAGCCTGGAGAGCGAGGAGCGGGGCGAGCTGGAG
CGGATCCTGGCCGACCTGGAGGAGGAGAACCGGAACCTGCAGGCCGAGTACGACCGGCTGAAGCAGCAGCACGAG
CACAAGGGCCTGAGCCCCCTGCCCAGCCCCCCCGAGATGATGCCCACCAGCCCCCAGAGCCCC
Nterm-R2_R23_R24 to end of dystrophin:
(SEQ ID NO: 35)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACCTGAGCCTGCAGGAGCTGCTGGTGTGGCTGCAGCTGAAGGACGACGAGCTGAGC
CGGCAGGCCCCCATCGGCGGCGACTTCCCCGCCGTGCAGAAGCAGAACGACGTGCACCGGGCCTTCAAGCGGGAG
CTGAAGACCAAGGAGCCCGTGATCATGAGCACCCTGGAGACCGTGCGGATCTTCCTGACCGAGCAGCCCCTGGAG
GGCCTGGAGAAGCTGTACCAGGAGCCCCGGGAGCTGCCCCCCGAGGAGCGGGCCCAGAACGTGACCCGGCTGCTG
CGGAAGCAGGCCGAGGAGGTGAACACCGAGTGGGAGAAGCTGAACCTGCACAGCGCCGACTGGCAGCGGAAGATC
GACGAGACCCTGGAGCGGCTGCAGGAGCTGCAGGAGGCCACCGACGAGCTGGACCTGAAGCTGCGGCAGGCCGAG
GTGATCAAGGGCAGCTGGCAGCCCGTGGGCGACCTGCTGATCGACAGCCTGCAGGACCACCTGGAGAAGGTGAAG
GCCCTGCGGGGCGAGATCGCCCCCCTGAAGGAGAACGTGAGCCACGTGAACGACCTGGCCCGGCAGCTGACCACC
CTGGGCATCCAGCTGAGCCCCTACAACCTGAGCACCCTGGAGGACCTGAACACCCGGTGGAAGCTGCTGCAGGTG
GCCGTGGAGGACCGGGTGCGGCAGCTGCACGAGGCCCACCGGGACTTCGGCCCCGCCAGCCAGCACTTCCTGAGC
ACCAGCGTGCAGGGCCCCTGGGAGCGGGCCATCAGCCCCAACAAGGTGCCCTACTACATCAACCACGAGACCCAG
ACCACCTGCTGGGACCACCCCAAGATGACCGAGCTGTACCAGAGCCTGGCCGACCTGAACAACGTGCGGTTCAGC
GCCTACCGGACCGCCATGAAGCTGCGGCGGCTGCAGAAGGCCCTGTGCCTGGACCTGCTGAGCCTGAGCGCCGCC
TGCGACGCCCTGGACCAGCACAACCTGAAGCAGAACGACCAGCCCATGGACATCCTGCAGATCATCAACTGCCTG
ACCACCATCTACGACCGGCTGGAGCAGGAGCACAACAACCTGGTGAACGTGCCCCTGTGCGTGGACATGTGCCTG
AACTGGCTGCTGAACGTGTACGACACCGGCCGGACCGGCCGGATCCGGGTGCTGAGCTTCAAGACCGGCATCATC
AGCCTGTGCAAGGCCCACCTGGAGGACAAGTACCGGTACCTGTTCAAGCAGGTGGCCAGCAGCACCGGCTTCTGC
GACCAGCGGCGGCTGGGCCTGCTGCTGCACGACAGCATCCAGATCCCCCGGCAGCTGGGCGAGGTGGCCAGCTTC
GGCGGCAGCAACATCGAGCCCAGCGTGCGGAGCTGCTTCCAGTTCGCCAACAACAAGCCCGAGATCGAGGCCGCC
CTGTTCCTGGACTGGATGCGGCTGGAGCCCCAGAGCATGGTGTGGCTGCCCGTGCTGCACCGGGTGGCCGCCGCC
GAGACCGCCAAGCACCAGGCCAAGTGCAACATCTGCAAGGAGTGCCCCATCATCGGCTTCCGGTACCGGAGCCTG
AAGCACTTCAACTACGACATCTGCCAGAGCTGCTTCTTCAGCGGCCGGGTGGCCAAGGGCCACAAGATGCACTAC
CCCATGGTGGAGTACTGCACCCCCACCACCAGCGGCGAGGACGTGCGGGACTTCGCCAAGGTGCTGAAGAACAAG
TTCCGGACCAAGCGGTACTTCGCCAAGCACCCCCGGATGGGCTACCTGCCCGTGCAGACCGTGCTGGAGGGCGAC
AACATGGAGACCCCC
GTGACCCTGATCAACTTCTGGCCCGTGGACAGCGCCCCCGCCAGCAGCCCCCAGCTGAGCCACGACGACACCCAC
AGCCGGATCGAGCACTACGCCAGCCGGCTGGCCGAGATGGAGAACAGCAACGGCAGCTACCTGAACGACAGCATC
AGCCCCAACGAGAGCATCGACGACGAGCACCTGCTGATCCAGCACTACTGCCAGAGCCTGAACCAGGACAGCCCC
CTGAGCCAGCCCCGGAGCCCCGCCCAGATCCTGATCAGCCTGGAGAGCGAGGAGCGGGGCGAGCTGGAGCGGATC
CTGGCCGACCTGGAGGAGGAGAACCGGAACCTGCAGGCCGAGTACGACCGGCTGAAGCAGCAGCACGAGCACAAG
GGCCTGAGCCCCCTGCCCAGCCCCCCCGAGATGATGCCCACCAGCCCCCAGAGCCCCCGGGACGCCGAGCTGATC
GCCGAGGCCAAGCTGCTGCGGCAGCACAAGGGCCGGCTGGAGGCCCGGATGCAGATCCTGGAGGACCACAACAAG
CAGCTGGAGAGCCAGCTGCACCGGCTGCGGCAGCTGCTGGAGCAGCCCCAGGCCGAGGCCAAGGTGAACGGCACC
ACCGTGAGCAGCCCCAGCACCAGCCTGCAGCGGAGCGACAGCAGCCAGCCCATGCTGCTGCGGGTGGTGGGCAGC
CAGACCAGCGACAGCATGGGCGAGGAGGACCTGCTGAGCCCCCCCCAGGACACCAGCACCGGCCTGGAGGAGGTG
ATGGAGCAGCTGAACAACAGCTTCCCCAGCAGCCGGGGCCGGAACACCCCCGGCAAGCCCATGCGGGAGGACACC
ATG
Nterm-R2_R23_R24 to end of dystrophin:
(SEQ ID NO: 134)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACGCCAGCAGCGACCAGTGGAAGCGGCTGCACCTGAGCCTGCAGGAGCTGCTGGTGTGG
CTGCAGCTGAAGGACGACGAGCTGAGCCGGCAGGCCCCCATCGGCGGCGACTTCCCCGCCGTGCAGAAGCAGAAC
GACGTGCACCGGGCCTTCAAGCGGGAGCTGAAGACCAAGGAGCCCGTGATCATGAGCACCCTGGAGACCGTGCGG
ATCTTCCTGACCGAGCAGCCCCTGGAGGGCCTGGAGAAGCTGTACCAGGAGCCCCGGGAGCTGCCCCCCGAGGAG
CGGGCCCAGAACGTGACCCGGCTGCTGCGGAAGCAGGCCGAGGAGGTGAACACCGAGTGGGAGAAGCTGAACCTG
CACAGCGCCGACTGGCAGCGGAAGATCGACGAGACCCTGGAGCGGCTGCAGGAGCTGCAGGAGGCCACCGACGAG
CTGGACCTGAAGCTGCGGCAGGCCGAGGTGATCAAGGGCAGCTGGCAGCCCGTGGGCGACCTGCTGATCGACAGC
CTGCAGGACCACCTGGAGAAGGTGAAGGCCCTGCGGGGCGAGATCGCCCCCCTGAAGGAGAACGTGAGCCACGTG
AACGACCTGGCCCGGCAGCTGACCACCCTGGGCATCCAGCTGAGCCCCTACAACCTGAGCACCCTGGAGGACCTG
AACACCCGGTGGAAGCTGCTGCAGGTGGCCGTGGAGGACCGGGTGCGGCAGCTGCACGAGGCCCACCGGGACTTC
GGCCCCGCCAGCCAGCACTTCCTGAGCACCAGCGTGCAGGGCCCCTGGGAGCGGGCCATCAGCCCCAACAAGGTG
CCCTACTACATCAACCACGAGACCCAGACCACCTGCTGGGACCACCCCAAGATGACCGAGCTGTACCAGAGCCTG
GCCGACCTGAACAACGTGCGGTTCAGCGCCTACCGGACCGCCATGAAGCTGCGGCGGCTGCAGAAGGCCCTGTGC
CTGGACCTGCTGAGCCTGAGCGCCGCCTGCGACGCCCTGGACCAGCACAACCTGAAGCAGAACGACCAGCCCATG
GACATCCTGCAGATCATCAACTGCCTGACCACCATCTACGACCGGCTGGAGCAGGAGCACAACAACCTGGTGAAC
GTGCCCCTGTGCGTGGACATGTGCCTGAACTGGCTGCTGAACGTGTACGACACCGGCCGGACCGGCCGGATCCGG
GTGCTGAGCTTCAAGACCGGCATCATCAGCCTGTGCAAGGCCCACCTGGAGGACAAGTACCGGTACCTGTTCAAG
CAGGTGGCCAGCAGCACCGGCTTCTGCGACCAGCGGCGGCTGGGCCTGCTGCTGCACGACAGCATCCAGATCCCC
CGGCAGCTGGGCGAGGTGGCCAGCTTCGGCGGCAGCAACATCGAGCCCAGCGTGCGGAGCTGCTTCCAGTTCGCC
AACAACAAGCCCGAGATCGAGGCCGCCCTGTTCCTGGACTGGATGCGGCTGGAGCCCCAGAGCATGGTGTGGCTG
CCCGTGCTGCACCGGGTGGCCGCCGCCGAGACCGCCAAGCACCAGGCCAAGTGCAACATCTGCAAGGAGTGCCCC
ATCATCGGCTTCCGGTACCGGAGCCTGAAGCACTTCAACTACGACATCTGCCAGAGCTGCTTCTTCAGCGGCCGG
GTGGCCAAGGGCCACAAGATGCACTACCCCATGGTGGAGTACTGCACCCCCACCACCAGCGGCGAGGACGTGCGG
GACTTCGCCAAGGTGCTGAAGAACAAGTTCCGGACCAAGCGGTACTTCGCCAAGCACCCCCGGATGGGCTACCTG
CCCGTGCAGACCGTGCTGGAGGGCGACAACATGGAGACCCCCGTGACCCTGATCAACTTCTGGCCCGTGGACAGC
GCCCCCGCCAGCAGCCCCCAGCTGAGCCACGACGACACCCACAGCCGGATCGAGCACTACGCCAGCCGGCTGGCC
GAGATGGAGAACAGCAACGGCAGCTACCTGAACGACAGCATCAGCCCCAACGAGAGCATCGACGACGAGCACCTG
CTGATCCAGCACTACTGCCAGAGCCTGAACCAGGACAGCCCCCTGAGCCAGCCCCGGAGCCCCGCCCAGATCCTG
ATCAGCCTGGAGAGCGAGGAGCGGGGCGAGCTGGAGCGGATCCTGGCCGACCTGGAGGAGGAGAACCGGAACCTG
CAGGCCGAGTACGACCGGCTGAAGCAGCAGCACGAGCACAAGGGCCTGAGCCCCCTGCCCAGCCCCCCCGAGATG
ATGCCCACCAGCCCCCAGAGCCCCCGGGACGCCGAGCTGATCGCCGAGGCCAAGCTGCTGCGGCAGCACAAGGGC
CGGCTGGAGGCCCGGATGCAGATCCTGGAGGACCACAACAAGCAGCTGGAGAGCCAGCTGCACCGGCTGCGGCAG
CTGCTGGAGCAGCCCCAGGCCGAGGCCAAGGTGAACGGCACCACCGTGAGCAGCCCCAGCACCAGCCTGCAGCGG
AGCGACAGCAGCCAGCCCATGCTGCTGCGGGTGGTGGGCAGCCAGACCAGCGACAGCATGGGCGAGGAGGACCTG
CTGAGCCCCCCCCAGGACACCAGCACCGGCCTGGAGGAGGTGATGGAGCAGCTGAACAACAGCTTCCCCAGCAGC
CGGGGCCGGAACACCCCCGGCAAGCCCATGCGGGAGGACACCATG
Nterm-R2_R16-R17_H4 to end:
(SEQ ID NO: 36)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACACCGTGCGGGAGGAGACCATGATGGTGATGACCGAGGACATGCCCCTGGAGATC
AGCTACGTGCCCAGCACCTACCTGACCGAGATCACCCACGTGAGCCAGGCCCTGCTGGAGGTGGAGCAGCTGCTG
AACGCCCCCGACCTGTGCGCCAAGGACTTCGAGGACCTGTTCAAGCAGGAGGAGAGCCTGAAGAACATCAAGGAC
AGCCTGCAGCAGAGCAGCGGCCGGATCGACATCATCCACAGCAAGAAGACCGCCGCCCTGCAGAGCGCCACCCCC
GTGGAGCGGGTGAAGCTGCAGGAGGCCCTGAGCCAGCTGGACTTCCAGTGGGAGAAGGTGAACAAGATGTACAAG
GACCGGCAGGGCCGGTTCGACCGGAGCGTGGAGAAGTGGCGGCGGTTCCACTACGACATCAAGATCTTCAACCAG
TGGCTGACCGAGGCCGAGCAGTTCCTGCGGAAGACCCAGATCCCCGAGAACTGGGAGCACGCCAAGTACAAGTGG
TACCTGAAGGAGCTGCAGGACGGCATCGGCCAGCGGCAGACCGTGGTGCGGACCCTGAACGCCACCGGCGAGGAG
ATCATCCAGCAGAGCAGCAAGACCGACGCCAGCATCCTGCAGGAGAAGCTGGGCAGCCTGAACCTGCGGTGGCAG
GAGGTGTGCAAGCAGCTGAGCGACCGGAAGAAGCGGCTGGAGGAGCAGAAGGGCCCCGCCAGCCAGCACTTCCTG
AGCACCAGCGTGCAGGGCCCCTGGGAGCGGGCCATCAGCCCCAACAAGGTGCCCTACTACATCAACCACGAGACC
CAGACCACCTGCTGGGACCACCCCAAGATGACCGAGCTGTACCAGAGCCTGGCCGACCTGAACAACGTGCGGTTC
AGCGCCTACCGGACCGCCATGAAGCTGCGGCGGCTGCAGAAGGCCCTGTGCCTGGACCTGCTGAGCCTGAGCGCC
GCCTGCGACGCCCTGGACCAGCACAACCTGAAGCAGAACGACCAGCCCATGGACATCCTGCAGATCATCAACTGC
CTGACCACCATCTACGACCGGCTGGAGCAGGAGCACAACAACCTGGTGAACGTGCCCCTGTGCGTGGACATGTGC
CTGAACTGGCTGCTGAACGTGTACGACACCGGCCGGACCGGCCGGATCCGGGTGCTGAGCTTCAAGACCGGCATC
ATCAGCCTGTGCAAGGCCCACCTGGAGGACAAGTACCGGTACCTGTTCAAGCAGGTGGCCAGCAGCACCGGCTTC
TGCGACCAGCGGCGGCTGGGCCTGCTGCTGCACGACAGCATCCAGATCCCCCGGCAGCTGGGCGAGGTGGCCAGC
TTCGGCGGCAGCAACATCGAGCCCAGCGTGCGGAGCTGCTTCCAGTTCGCCAACAACAAGCCCGAGATCGAGGCC
GCCCTGTTCCTGGACTGGATGCGGCTGGAGCCCCAGAGCATGGTGTGGCTGCCCGTGCTGCACCGGGTGGCCGCC
GCCGAGACCGCCAAGCACCAGGCCAAGTGCAACATCTGCAAGGAGTGCCCCATCATCGGCTTCCGGTACCGGAGC
CTGAAGCACTTCAACTACGACATCTGCCAGAGCTGCTTCTTCAGCGGCCGGGTGGCCAAGGGCCACAAGATGCAC
TACCCCATGGTGGAGTACTGCACCCCCACCACCAGCGGCGAGGACGTGCGGGACTTCGCCAAGGTGCTGAAGAAC
AAGTTCCGGACCAAGCGGTACTTCGCCAAGCACCCCCGGATGGGCTACCTGCCCGTGCAGACCGTGCTGGAGGGC
GACAACATGGAGACCCCCGTGACCCTGATCAACTTCTGGCCCGTGGACAGCGCCCCCGCCAGCAGCCCCCAGCTG
AGCCACGACGACACCCACAGCCGGATCGAGCACTACGCCAGCCGGCTGGCCGAGATGGAGAACAGCAACGGCAGC
TACCTGAACGACAGCATCAGCCCCAACGAGAGCATCGACGACGAGCACCTGCTGATCCAGCACTACTGCCAGAGC
CTGAACCAGGACAGCCCCCTGAGCCAGCCCCGGAGCCCCGCCCAGATCCTGATCAGCCTGGAGAGCGAGGAGCGG
GGCGAGCTGGAGCGGATCCTGGCCGACCTGGAGGAGGAGAACCGGAACCTGCAGGCCGAGTACGACCGGCTGAAG
CAGCAGCACGAGCACAAGGGCCTGAGCCCCCTGCCCAGCCCCCCCGAGATGATGCCCACCAGCCCCCAGAGCCCC
CGGGACGCCGAGCTGATCGCCGAGGCCAAGCTGCTGCGGCAGCACAAGGGCCGGCTGGAGGCCCGGATGCAGATC
CTGGAGGACCACAACAAGCAGCTGGAGAGCCAGCTGCACCGGCTGCGGCAGCTGCTGGAGCAGCCCCAGGCCGAG
GCCAAGGTGAACGGCACCACCGTGAGCAGCCCCAGCACCAGCCTGCAGCGGAGCGACAGCAGCCAGCCCATGCTG
CTGCGGGTGGTGGGCAGCCAGACCAGCGACAGCATGGGCGAGGAGGACCTGCTGAGCCCCCCCCAGGACACCAGC
ACCGGCCTGGAGGAGGTGATGGAGCAGCTGAACAACAGCTTCCCCAGCAGCCGGGGCCGGAACACCCCCGGCAAG
CCCATGCGGGAGGACACCATG
Nterm-R1_R16-R17_R24 to end:
(SEQ ID NO: 37)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCACACCGTGCGGGAGGAGACCATGATGGTGATGACCGAGGACATGCCCCTGGAGATCAGCTACGTGCCCAGC
ACCTACCTGACCGAGATCACCCACGTGAGCCAGGCCCTGCTGGAGGTGGAGCAGCTGCTGAACGCCCCCGACCTG
TGCGCCAAGGACTTCGAGGACCTGTTCAAGCAGGAGGAGAGCCTGAAGAACATCAAGGACAGCCTGCAGCAGAGC
AGCGGCCGGATCGACATCATCCACAGCAAGAAGACCGCCGCCCTGCAGAGCGCCACCCCCGTGGAGCGGGTGAAG
CTGCAGGAGGCCCTGAGCCAGCTGGACTTCCAGTGGGAGAAGGTGAACAAGATGTACAAGGACCGGCAGGGCCGG
TTCGACCGGAGCGTGGAGAAGTGGCGGCGGTTCCACTACGACATCAAGATCTTCAACCAGTGGCTGACCGAGGCC
GAGCAGTTCCTGCGGAAGACCCAGATCCCCGAGAACTGGGAGCACGCCAAGTACAAGTGGTACCTGAAGGAGCTG
CAGGACGGCATCGGCCAGCGGCAGACCGTGGTGCGGACCCTGAACGCCACCGGCGAGGAGATCATCCAGCAGAGC
AGCAAGACCGACGCCAGCATCCTGCAGGAGAAGCTGGGCAGCCTGAACCTGCGGTGGCAGGAGGTGTGCAAGCAG
CTGAGCGACCGGAAGAAGCGGCTGGAGGAGCAGAAGTGGCAGCGGAAGATCGACGAGACCCTGGAGCGGCTGCAG
GAGCTGCAGGAGGCCACCGACGAGCTGGACCTGAAGCTGCGGCAGGCCGAGGTGATCAAGGGCAGCTGGCAGCCC
GTGGGCGACCTGCTGATCGACAGCCTGCAGGACCACCTGGAGAAGGTGAAGGCCCTGCGGGGCGAGATCGCCCCC
CTGAAGGAGAACGTGAGCCACGTGAACGACCTGGCCCGGCAGCTGACCACCCTGGGCATCCAGCTGAGCCCCTAC
AACCTGAGCACCCTGGAGGACCTGAACACCCGGTGGAAGCTGCTGCAGGTGGCCGTGGAGGACCGGGTGCGGCAG
CTGCACGAGGCCCACCGGGACTTCGGCCCCGCCAGCCAGCACTTCCTGAGCACCAGCGTGCAGGGCCCCTGGGAG
CGGGCCATCAGCCCCAACAAGGTGCCCTACTACATCAACCACGAGACCCAGACCACCTGCTGGGACCACCCCAAG
ATGACCGAGCTGTACCAGAGCCTGGCCGACCTGAACAACGTGCGGTTCAGCGCCTACCGGACCGCCATGAAGCTG
CGGCGGCTGCAGAAGGCCCTGTGCCTGGACCTGCTGAGCCTGAGCGCCGCCTGCGACGCCCTGGACCAGCACAAC
CTGAAGCAGAACGACCAGCCCATGGACATCCTGCAGATCATCAACTGCCTGACCACCATCTACGACCGGCTGGAG
CAGGAGCACAACAACCTGGTGAACGTGCCCCTGTGCGTGGACATGTGCCTGAACTGGCTGCTGAACGTGTACGAC
ACCGGCCGGACCGGCCGGATCCGGGTGCTGAGCTTCAAGACCGGCATCATCAGCCTGTGCAAGGCCCACCTGGAG
GACAAGTACCGGTACCTGTTCAAGCAGGTGGCCAGCAGCACCGGCTTCTGCGACCAGCGGCGGCTGGGCCTGCTG
CTGCACGACAGCATCCAGATCCCCCGGCAGCTGGGCGAGGTGGCCAGCTTCGGCGGCAGCAACATCGAGCCCAGC
GTGCGGAGCTGCTTCCAGTTCGCCAACAACAAGCCCGAGATCGAGGCCGCCCTGTTCCTGGACTGGATGCGGCTG
GAGCCCCAGAGCATGGTGTGGCTGCCCGTGCTGCACCGGGTGGCCGCCGCCGAGACCGCCAAGCACCAGGCCAAG
TGCAACATCTGCAAGGAGTGCCCCATCATCGGCTTCCGGTACCGGAGCCTGAAGCACTTCAACTACGACATCTGC
CAGAGCTGCTTCTTCAGCGGCCGGGTGGCCAAGGGCCACAAGATGCACTACCCCATGGTGGAGTACTGCACCCCC
ACCACCAGCGGCGAGGACGTGCGGGACTTCGCCAAGGTGCTGAAGAACAAGTTCCGGACCAAGCGGTACTTCGCC
AAGCACCCCCGGATGGGCTACCTGCCCGTGCAGACCGTGCTGGAGGGCGACAACATGGAGACCCCCGTGACCCTG
ATCAACTTCTGGCCCGTGGACAGCGCCCCCGCCAGCAGCCCCCAGCTGAGCCACGACGACACCCACAGCCGGATC
GAGCACTACGCCAGCCGGCTGGCCGAGATGGAGAACAGCAACGGCAGCTACCTGAACGACAGCATCAGCCCCAAC
GAGAGCATCGACGACGAGCACCTGCTGATCCAGCACTACTGCCAGAGCCTGAACCAGGACAGCCCCCTGAGCCAG
CCCCGGAGCCCCGCCCAGATCCTGATCAGCCTGGAGAGCGAGGAGCGGGGCGAGCTGGAGCGGATCCTGGCCGAC
CTGGAGGAGGAGAACCGGAACCTGCAGGCCGAGTACGACCGGCTGAAGCAGCAGCACGAGCACAAGGGCCTGAGC
CCCCTGCCCAGCCCCCCCGAGATGATGCCCACCAGCCCCCAGAGCCCCCGGGACGCCGAGCTGATCGCCGAGGCC
AAGCTGCTGCGGCAGCACAAGGGCCGGCTGGAGGCCCGGATGCAGATCCTGGAGGACCACAACAAGCAGCTGGAG
AGCCAGCTGCACCGGCTGCGGCAGCTGCTGGAGCAGCCCCAGGCCGAGGCCAAGGTGAACGGCACCACCGTGAGC
AGCCCCAGCACCAGCCTGCAGCGGAGCGACAGCAGCCAGCCCATGCTGCTGCGGGTGGTGGGCAGCCAGACCAGC
GACAGCATGGGCGAGGAGGACCTGCTGAGCCCCCCCCAGGACACCAGCACCGGCCTGGAGGAGGTGATGGAGCAG
CTGAACAACAGCTTCCCCAGCAGCCGGGGCCGGAACACCCCCGGCAAGCCCATGCGGGAGGACACCATG
Nterm-R2_R16-R17_R24 to end:
(SEQ ID NO: 38)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACACCGTGCGGGAGGAGACCATGATGGTGATGACCGAGGACATGCCCCTGGAGATC
AGCTACGTGCCCAGCACCTACCTGACCGAGATCACCCACGTGAGCCAGGCCCTGCTGGAGGTGGAGCAGCTGCTG
AACGCCCCCGACCTGTGCGCCAAGGACTTCGAGGACCTGTTCAAGCAGGAGGAGAGCCTGAAGAACATCAAGGAC
AGCCTGCAGCAGAGCAGCGGCCGGATCGACATCATCCACAGCAAGAAGACCGCCGCCCTGCAGAGCGCCACCCCC
GTGGAGCGGGTGAAGCTGCAGGAGGCCCTGAGCCAGCTGGACTTCCAGTGGGAGAAGGTGAACAAGATGTACAAG
GACCGGCAGGGCCGGTTCGACCGGAGCGTGGAGAAGTGGCGGCGGTTCCACTACGACATCAAGATCTTCAACCAG
TGGCTGACCGAGGCCGAGCAGTTCCTGCGGAAGACCCAGATCCCCGAGAACTGGGAGCACGCCAAGTACAAGTGG
TACCTGAAGGAGCTGCAGGACGGCATCGGCCAGCGGCAGACCGTGGTGCGGACCCTGAACGCCACCGGCGAGGAG
ATCATCCAGCAGAGCAGCAAGACCGACGCCAGCATCCTGCAGGAGAAGCTGGGCAGCCTGAACCTGCGGTGGCAG
GAGGTGTGCAAGCAGCTGAGCGACCGGAAGAAGCGGCTGGAGGAGCAGAAGTGGCAGCGGAAGATCGACGAGACC
CTGGAGCGGCTGCAGGAGCTGCAGGAGGCCACCGACGAGCTGGACCTGAAGCTGCGGCAGGCCGAGGTGATCAAG
GGCAGCTGGCAGCCCGTGGGCGACCTGCTGATCGACAGCCTGCAGGACCACCTGGAGAAGGTGAAGGCCCTGCGG
GGCGAGATCGCCCCCCTGAAGGAGAACGTGAGCCACGTGAACGACCTGGCCCGGCAGCTGACCACCCTGGGCATC
CAGCTGAGCCCCTACAACCTGAGCACCCTGGAGGACCTGAACACCCGGTGGAAGCTGCTGCAGGTGGCCGTGGAG
GACCGGGTGCGGCAGCTGCACGAGGCCCACCGGGACTTCGGCCCCGCCAGCCAGCACTTCCTGAGCACCAGCGTG
CAGGGCCCCTGGGAGCGGGCCATCAGCCCCAACAAGGTGCCCTACTACATCAACCACGAGACCCAGACCACCTGC
TGGGACCACCCCAAGATGACCGAGCTGTACCAGAGCCTGGCCGACCTGAACAACGTGCGGTTCAGCGCCTACCGG
ACCGCCATGAAGCTGCGGCGGCTGCAGAAGGCCCTGTGCCTGGACCTGCTGAGCCTGAGCGCCGCCTGCGACGCC
CTGGACCAGCACAACCTGAAGCAGAACGACCAGCCCATGGACATCCTGCAGATCATCAACTGCCTGACCACCATC
TACGACCGGCTGGAGCAGGAGCACAACAACCTGGTGAACGTGCCCCTGTGCGTGGACATGTGCCTGAACTGGCTG
CTGAACGTGTACGACACCGGCCGGACCGGCCGGATCCGGGTGCTGAGCTTCAAGACCGGCATCATCAGCCTGTGC
AAGGCCCACCTGGAGGACAAGTACCGGTACCTGTTCAAGCAGGTGGCCAGCAGCACCGGCTTCTGCGACCAGCGG
CGGCTGGGCCTGCTGCTGCACGACAGCATCCAGATCCCCCGGCAGCTGGGCGAGGTGGCCAGCTTCGGCGGCAGC
AACATCGAGCCCAGCGTGCGGAGCTGCTTCCAGTTCGCCAACAACAAGCCCGAGATCGAGGCCGCCCTGTTCCTG
GACTGGATGCGGCTGGAGCCCCAGAGCATGGTGTGGCTGCCCGTGCTGCACCGGGTGGCCGCCGCCGAGACCGCC
AAGCACCAGGCCAAGTGCAACATCTGCAAGGAGTGCCCCATCATCGGCTTCCGGTACCGGAGCCTGAAGCACTTC
AACTACGACATCTGCCAGAGCTGCTTCTTCAGCGGCCGGGTGGCCAAGGGCCACAAGATGCACTACCCCATGGTG
GAGTACTGCACCCCCACCACCAGCGGCGAGGACGTGCGGGACTTCGCCAAGGTGCTGAAGAACAAGTTCCGGACC
AAGCGGTACTTCGCCAAGCACCCCCGGATGGGCTACCTGCCCGTGCAGACCGTGCTGGAGGGCGACAACATGGAG
ACCCCCGTGACCCTGATCAACTTCTGGCCCGTGGACAGCGCCCCCGCCAGCAGCCCCCAGCTGAGCCACGACGAC
ACCCACAGCCGGATCGAGCACTACGCCAGCCGGCTGGCCGAGATGGAGAACAGCAACGGCAGCTACCTGAACGAC
AGCATCAGCCCCAACGAGAGCATCGACGACGAGCACCTGCTGATCCAGCACTACTGCCAGAGCCTGAACCAGGAC
AGCCCCCTGAGCCAGCCCCGGAGCCCCGCCCAGATCCTGATCAGCCTGGAGAGCGAGGAGCGGGGCGAGCTGGAG
CGGATCCTGGCCGACCTGGAGGAGGAGAACCGGAACCTGCAGGCCGAGTACGACCGGCTGAAGCAGCAGCACGAG
CACAAGGGCCTGAGCCCCCTGCCCAGCCCCCCCGAGATGATGCCCACCAGCCCCCAGAGCCCCCGGGACGCCGAG
CTGATCGCCGAGGCCAAGCTGCTGCGGCAGCACAAGGGCCGGCTGGAGGCCCGGATGCAGATCCTGGAGGACCAC
AACAAGCAGCTGGAGAGCCAGCTGCACCGGCTGCGGCAGCTGCTGGAGCAGCCCCAGGCCGAGGCCAAGGTGAAC
GGCACCACCGTGAGCAGCCCCAGCACCAGCCTGCAGCGGAGCGACAGCAGCCAGCCCATGCTGCTGCGGGTGGTG
GGCAGCCAGACCAGCGACAGCATGGGCGAGGAGGACCTGCTGAGCCCCCCCCAGGACACCAGCACCGGCCTGGAG
GAGGTGATGGAGCAGCTGAACAACAGCTTCCCCAGCAGCCGGGGCCGGAACACCCCCGGCAAGCCCATGCGGGAG
GACACCATG
Nterm-R2_R16-R17_R24 to end of syntrophin region:
(SEQ ID NO: 39)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GACCTGCAGAACCAGAAGCTGAAGGAGCTGAACGACTGGCTGACCAAGACCGAGGAGCGGACCCGGAAGATGGAG
GAGGAGCCCCTGGGCCCCGACCTGGAGGACCTGAAGCGGCAGGTGCAGCAGCACAAGGTGCTGCAGGAGGACCTG
GAGCAGGAGCAGGTGCGGGTGAACAGCCTGACCCACATGGTGGTGGTGGTGGACGAGAGCAGCGGCGACCACGCC
ACCGCCGCCCTGGAGGAGCAGCTGAAGGTGCTGGGCGACCGGTGGGCCAACATCTGCCGGTGGACCGAGGACCGG
TGGGTGCTGCTGCAGGACCACACCGTGCGGGAGGAGACCATGATGGTGATGACCGAGGACATGCCCCTGGAGATC
AGCTACGTGCCCAGCACCTACCTGACCGAGATCACCCACGTGAGCCAGGCCCTGCTGGAGGTGGAGCAGCTGCTG
AACGCCCCCGACCTGTGCGCCAAGGACTTCGAGGACCTGTTCAAGCAGGAGGAGAGCCTGAAGAACATCAAGGAC
AGCCTGCAGCAGAGCAGCGGCCGGATCGACATCATCCACAGCAAGAAGACCGCCGCCCTGCAGAGCGCCACCCCC
GTGGAGCGGGTGAAGCTGCAGGAGGCCCTGAGCCAGCTGGACTTCCAGTGGGAGAAGGTGAACAAGATGTACAAG
GACCGGCAGGGCCGGTTCGACCGGAGCGTGGAGAAGTGGCGGCGGTTCCACTACGACATCAAGATCTTCAACCAG
TGGCTGACCGAGGCCGAGCAGTTCCTGCGGAAGACCCAGATCCCCGAGAACTGGGAGCACGCCAAGTACAAGTGG
TACCTGAAGGAGCTGCAGGACGGCATCGGCCAGCGGCAGACCGTGGTGCGGACCCTGAACGCCACCGGCGAGGAG
ATCATCCAGCAGAGCAGCAAGACCGACGCCAGCATCCTGCAGGAGAAGCTGGGCAGCCTGAACCTGCGGTGGCAG
GAGGTGTGCAAGCAGCTGAGCGACCGGAAGAAGCGGCTGGAGGAGCAGAAGTGGCAGCGGAAGATCGACGAGACC
CTGGAGCGGCTGCAGGAGCTGCAGGAGGCCACCGACGAGCTGGACCTGAAGCTGCGGCAGGCCGAGGTGATCAAG
GGCAGCTGGCAGCCCGTGGGCGACCTGCTGATCGACAGCCTGCAGGACCACCTGGAGAAGGTGAAGGCCCTGCGG
GGCGAGATCGCCCCCCTGAAGGAGAACGTGAGCCACGTGAACGACCTGGCCCGGCAGCTGACCACCCTGGGCATC
CAGCTGAGCCCCTACAACCTGAGCACCCTGGAGGACCTGAACACCCGGTGGAAGCTGCTGCAGGTGGCCGTGGAG
GACCGGGTGCGGCAGCTGCACGAGGCCCACCGGGACTTCGGCCCCGCCAGCCAGCACTTCCTGAGCACCAGCGTG
CAGGGCCCCTGGGAGCGGGCCATCAGCCCCAACAAGGTGCCCTACTACATCAACCACGAGACCCAGACCACCTGC
TGGGACCACCCCAAGATGACCGAGCTGTACCAGAGCCTGGCCGACCTGAACAACGTGCGGTTCAGCGCCTACCGG
ACCGCCATGAAGCTGCGGCGGCTGCAGAAGGCCCTGTGCCTGGACCTGCTGAGCCTGAGCGCCGCCTGCGACGCC
CTGGACCAGCACAACCTGAAGCAGAACGACCAGCCCATGGACATCCTGCAGATCATCAACTGCCTGACCACCATC
TACGACCGGCTGGAGCAGGAGCACAACAACCTGGTGAACGTGCCCCTGTGCGTGGACATGTGCCTGAACTGGCTG
CTGAACGTGTACGACACCGGCCGGACCGGCCGGATCCGGGTGCTGAGCTTCAAGACCGGCATCATCAGCCTGTGC
AAGGCCCACCTGGAGGACAAGTACCGGTACCTGTTCAAGCAGGTGGCCAGCAGCACCGGCTTCTGCGACCAGCGG
CGGCTGGGCCTGCTGCTGCACGACAGCATCCAGATCCCCCGGCAGCTGGGCGAGGTGGCCAGCTTCGGCGGCAGC
AACATCGAGCCCAGCGTGCGGAGCTGCTTCCAGTTCGCCAACAACAAGCCCGAGATCGAGGCCGCCCTGTTCCTG
GACTGGATGCGGCTGGAGCCCCAGAGCATGGTGTGGCTGCCCGTGCTGCACCGGGTGGCCGCCGCCGAGACCGCC
AAGCACCAGGCCAAGTGCAACATCTGCAAGGAGTGCCCCATCATCGGCTTCCGGTACCGGAGCCTGAAGCACTTC
AACTACGACATCTGCCAGAGCTGCTTCTTCAGCGGCCGGGTGGCCAAGGGCCACAAGATGCACTACCCCATGGTG
GAGTACTGCACCCCCACCACCAGCGGCGAGGACGTGCGGGACTTCGCCAAGGTGCTGAAGAACAAGTTCCGGACC
AAGCGGTACTTCGCCAAGCACCCCCGGATGGGCTACCTGCCCGTGCAGACCGTGCTGGAGGGCGACAACATGGAG
ACCCCCGTGACCCTGATCAACTTCTGGCCCGTGGACAGCGCCCCCGCCAGCAGCCCCCAGCTGAGCCACGACGAC
ACCCACAGCCGGATCGAGCACTACGCCAGCCGGCTGGCCGAGATGGAGAACAGCAACGGCAGCTACCTGAACGAC
AGCATCAGCCCCAACGAGAGCATCGACGACGAGCACCTGCTGATCCAGCACTACTGCCAGAGCCTGAACCAGGAC
AGCCCCCTGAGCCAGCCCCGGAGC

In some embodiments, a micro-dystrophin protein as described herein further comprises one or more utrophin protein domains. Non-limiting examples of nucleotide sequences encoding human chimeric utrophin-dystrophin micro-dystrophin proteins optimized for skeletal muscle are described below.

Utrophin N-terminus through R2_Dystrophin R16-R17_R24 + H4 + trCterm:
Human sequences without codon alterations:
(SEQ ID NO: 102)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCGCCGCTAGCGCCATGATGGTGATGACTGAAGACATGCCTTTGGAAATTTCTTATGTGCCTTCTACTTAT
TTGACTGAAATCACTCATGTCTCACAAGCCCTATTAGAAGTGGAACAACTTCTCAATGCTCCTGACCTCTGTGCT
AAGGACTTTGAAGATCTCTTTAAGCAAGAGGAGTCTCTGAAGAATATAAAAGATAGTCTACAACAAAGCTCAGGT
CGGATTGACATTATTCATAGCAAGAAGACAGCAGCATTGCAAAGTGCAACGCCTGTGGAAAGGGTGAAGCTACAG
GAAGCTCTCTCCCAGCTTGATTTCCAATGGGAAAAAGTTAACAAAATGTACAAGGACCGACAAGGGCGATTTGAC
AGATCTGTTGAGAAATGGCGGCGTTTTCATTATGATATAAAGATATTTAATCAGTGGCTAACAGAAGCTGAACAG
TTTCTCAGAAAGACACAAATTCCTGAGAATTGGGAACATGCTAAATACAAATGGTATCTTAAGGAACTCCAGGAT
GGCATTGGGCAGCGGCAAACTGTTGTCAGAACATTGAATGCAACTGGGGAAGAAATAATTCAGCAATCCTCAAAA
ACAGATGCCAGTATTCTACAGGAAAAATTGGGAAGCCTGAATCTGCGGTGGCAGGAGGTCTGCAAACAGCTGTCA
GACAGAAAAAAGAGGCTAGAAGAACAAAAGTGGCAGAGAAAAATAGATGAGACCCTTGAAAGACTCCGGGAACTT
CAAGAGGCCACGGATGAGCTGGACCTCAAGCTGCGCCAAGCTGAGGTGATCAAGGGATCCTGGCAGCCCGTGGGC
GATCTCCTCATTGACTCTCTCCAAGATCACCTCGAGAAAGTCAAGGCACTTCGAGGAGAAATTGCGCCTCTGAAA
GAGAACGTGAGCCACGTCAATGACCTTGCTCGCCAGCTTACCACTTTGGGCATTCAGCTCTCACCGTATAACCTC
AGCACTCTGGAAGACCTGAACACCAGATGGAAGCTTCTGCAGGTGGCCGTCGAGGACCGAGTCAGGCAGCTGCAT
GAAGCCCACAGGGACTTTGGTCCAGCATCTCAGCACTTTCTTTCCACGTCTGTCCAGGGTCCCTGGGAGAGAGCC
ATCTCGCCAAACAAAGTGCCCTACTATATCAACCACGAGACTCAAACAACTTGCTGGGACCATCCCAAAATGACA
GAGCTCTACCAGTCTTTAGCTGACCTGAATAATGTCAGATTCTCAGCTTATAGGACTGCCATGAAACTCCGAAGA
CTGCAGAAGGCCCTTTGCTTGGATCTCTTGAGCCTGTCAGCTGCATGTGATGCCTTGGACCAGCACAACCTCAAG
CAAAATGACCAGCCCATGGATATCCTGCAGATTATTAATTGTTTGACCACTATTTATGACCGCCTGGAGCAAGAG
CACAACAATTTGGTCAACGTCCCTCTCTGCGTGGATATGTGTCTGAACTGGCTGCTGAATGTTTATGATACGGGA
CGAACAGGGAGGATCCGTGTCCTGTCTTTTAAAACTGGCATCATTTCCCTGTGTAAAGCACATTTGGAAGACAAG
TACAGATACCTTTTCAAGCAAGTGGCAAGTTCAACAGGATTTTGTGACCAGCGCAGGCTGGGCCTCCTTCTGCAT
GATTCTATCCAAATTCCAAGACAGTTGGGTGAAGTTGCATCCTTTGGGGGCAGTAACATTGAGCCAAGTGTCCGG
AGCTGCTTCCAATTTGCTAATAATAAGCCAGAGATCGAAGCGGCCCTCTTCCTAGACTGGATGAGACTGGAACCC
CAGTCCATGGTGTGGCTGCCCGTCCTGCACAGAGTGGCTGCTGCAGAAACTGCCAAGCATCAGGCCAAATGTAAC
ATCTGCAAAGAGTGTCCAATCATTGGATTCAGGTACAGGAGTCTAAAGCACTTTAATTATGACATCTGCCAAAGC
TGCTTTTTTTCTGGTCGAGTTGCAAAAGGCCATAAAATGCACTATCCCATGGTGGAATATTGCACTCCGACTACA
TCAGGAGAAGATGTTCGAGACTTTGCCAAGGTACTAAAAAACAAATTTCGAACCAAAAGGTATTTTGCGAAGCAT
CCCCGAATGGGCTACCTGCCAGTGCAGACTGTCTTAGAGGGGGACAACATGGAAACTCCC
Human sequences with codon optimization:
(SEQ ID NO: 103)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCGCCGCTAGCGCCATGATGGTGATGACCGAGGACATGCCCCTGGAGATCAGCTACGTGCCCAGCACCTAC
CTGACCGAGATCACCCACGTGAGCCAGGCCCTGCTGGAGGTGGAGCAGCTGCTGAACGCCCCCGACCTGTGCGCC
AAGGACTTCGAGGACCTGTTCAAGCAGGAGGAGAGCCTGAAGAACATCAAGGACAGCCTGCAGCAGAGCAGCGGC
CGGATCGACATCATCCACAGCAAGAAGACCGCCGCCCTGCAGAGCGCCACCCCCGTGGAGCGGGTGAAGCTGCAG
GAGGCCCTGAGCCAGCTGGACTTCCAGTGGGAGAAGGTGAACAAGATGTACAAGGACCGGCAGGGCCGGTTCGAC
CGGAGCGTGGAGAAGTGGCGGCGGTTCCACTACGACATCAAGATCTTCAACCAGTGGCTGACCGAGGCCGAGCAG
TTCCTGCGGAAGACCCAGATCCCCGAGAACTGGGAGCACGCCAAGTACAAGTGGTACCTGAAGGAGCTGCAGGAC
GGCATCGGCCAGCGGCAGACCGTGGTGCGGACCCTGAACGCCACCGGCGAGGAGATCATCCAGCAGAGCAGCAAG
ACCGACGCCAGCATCCTGCAGGAGAAGCTGGGCAGCCTGAACCTGCGGTGGCAGGAGGTGTGCAAGCAGCTGAGC
GACCGGAAGAAGCGGCTGGAGGAGCAGAAGTGGCAGCGGAAGATCGACGAGACCCTGGAGCGGCTGCAGGAGCTG
CAGGAGGCCACCGACGAGCTGGACCTGAAGCTGCGGCAGGCCGAGGTGATCAAGGGCAGCTGGCAGCCCGTGGGC
GACCTGCTGATCGACAGCCTGCAGGACCACCTGGAGAAGGTGAAGGCCCTGCGGGGCGAGATCGCCCCCCTGAAG
GAGAACGTGAGCCACGTGAACGACCTGGCCCGGCAGCTGACCACCCTGGGCATCCAGCTGAGCCCCTACAACCTG
AGCACCCTGGAGGACCTGAACACCCGGTGGAAGCTGCTGCAGGTGGCCGTGGAGGACCGGGTGCGGCAGCTGCAC
GAGGCCCACCGGGACTTCGGCCCCGCCAGCCAGCACTTCCTGAGCACCAGCGTGCAGGGCCCCTGGGAGCGGGCC
ATCAGCCCCAACAAGGTGCCCTACTACATCAACCACGAGACCCAGACCACCTGCTGGGACCACCCCAAGATGACC
GAGCTGTACCAGAGCCTGGCCGACCTGAACAACGTGCGGTTCAGCGCCTACCGGACCGCCATGAAGCTGCGGCGG
CTGCAGAAGGCCCTGTGCCTGGACCTGCTGAGCCTGAGCGCCGCCTGCGACGCCCTGGACCAGCACAACCTGAAG
CAGAACGACCAGCCCATGGACATCCTGCAGATCATCAACTGCCTGACCACCATCTACGACCGGCTGGAGCAGGAG
CACAACAACCTGGTGAACGTGCCCCTGTGCGTGGACATGTGCCTGAACTGGCTGCTGAACGTGTACGACACCGGC
CGGACCGGCCGGATCCGGGTGCTGAGCTTCAAGACCGGCATCATCAGCCTGTGCAAGGCCCACCTGGAGGACAAG
TACCGGTACCTGTTCAAGCAGGTGGCCAGCAGCACCGGCTTCTGCGACCAGCGGCGGCTGGGCCTGCTGCTGCAC
GACAGCATCCAGATCCCCCGGCAGCTGGGCGAGGTGGCCAGCTTCGGCGGCAGCAACATCGAGCCCAGCGTGCGG
AGCTGCTTCCAGTTCGCCAACAACAAGCCCGAGATCGAGGCCGCCCTGTTCCTGGACTGGATGCGGCTGGAGCCC
CAGAGCATGGTGTGGCTGCCCGTGCTGCACCGGGTGGCCGCCGCCGAGACCGCCAAGCACCAGGCCAAGTGCAAC
ATCTGCAAGGAGTGCCCCATCATCGGCTTCCGGTACCGGAGCCTGAAGCACTTCAACTACGACATCTGCCAGAGC
TGCTTCTTCAGCGGCCGGGTGGCCAAGGGCCACAAGATGCACTACCCCATGGTGGAGTACTGCACCCCCACCACC
AGCGGCGAGGACGTGCGGGACTTCGCCAAGGTGCTGAAGAACAAGTTCCGGACCAAGCGGTACTTCGCCAAGCAC
CCCCGGATGGGCTACCTGCCCGTGCAGACCGTGCTGGAGGGCGACAACATGGAGACCCCC
Utrophin N-terminus through R2_Dystrophin R16-R17_H4 to end of syntrophin region:
Human sequences without codon alterations:
(SEQ ID NO: 104)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCGCCGCTAGCGCCATGATGGTGATGACTGAAGACATGCCTTTGGAAATTTCTTATGTGCCTTCTACTTAT
TTGACTGAAATCACTCATGTCTCACAAGCCCTATTAGAAGTGGAACAACTTCTCAATGCTCCTGACCTCTGTGCT
AAGGACTTTGAAGATCTCTTTAAGCAAGAGGAGTCTCTGAAGAATATAAAAGATAGTCTACAACAAAGCTCAGGT
CGGATTGACATTATTCATAGCAAGAAGACAGCAGCATTGCAAAGTGCAACGCCTGTGGAAAGGGTGAAGCTACAG
GAAGCTCTCTCCCAGCTTGATTTCCAATGGGAAAAAGTTAACAAAATGTACAAGGACCGACAAGGGCGATTTGAC
AGATCTGTTGAGAAATGGCGGCGTTTTCATTATGATATAAAGATATTTAATCAGTGGCTAACAGAAGCTGAACAG
TTTCTCAGAAAGACACAAATTCCTGAGAATTGGGAACATGCTAAATACAAATGGTATCTTAAGGAACTCCAGGAT
GGCATTGGGCAGCGGCAAACTGTTGTCAGAACATTGAATGCAACTGGGGAAGAAATAATTCAGCAATCCTCAAAA
ACAGATGCCAGTATTCTACAGGAAAAATTGGGAAGCCTGAATCTGCGGTGGCAGGAGGTCTGCAAACAGCTGTCA
GACAGAAAAAAGAGGCTAGAAGAACAAAAGGGTCCAGCATCTCAGCACTTTCTTTCCACGTCTGTCCAGGGTCCC
TGGGAGAGAGCCATCTCGCCAAACAAAGTGCCCTACTATATCAACCACGAGACTCAAACAACTTGCTGGGACCAT
CCCAAAATGACAGAGCTCTACCAGTCTTTAGCTGACCTGAATAATGTCAGATTCTCAGCTTATAGGACTGCCATG
AAACTCCGAAGACTGCAGAAGGCCCTTTGCTTGGATCTCTTGAGCCTGTCAGCTGCATGTGATGCCTTGGACCAG
CACAACCTCAAGCAAAATGACCAGCCCATGGATATCCTGCAGATTATTAATTGTTTGACCACTATTTATGACCGC
CTGGAGCAAGAGCACAACAATTTGGTCAACGTCCCTCTCTGCGTGGATATGTGTCTGAACTGGCTGCTGAATGTT
TATGATACGGGACGAACAGGGAGGATCCGTGTCCTGTCTTTTAAAACTGGCATCATTTCCCTGTGTAAAGCACAT
TTGGAAGACAAGTACAGATACCTTTTCAAGCAAGTGGCAAGTTCAACAGGATTTTGTGACCAGCGCAGGCTGGGC
CTCCTTCTGCATGATTCTATCCAAATTCCAAGACAGTTGGGTGAAGTTGCATCCTTTGGGGGCAGTAACATTGAG
CCAAGTGTCCGGAGCTGCTTCCAATTTGCTAATAATAAGCCAGAGATCGAAGCGGCCCTCTTCCTAGACTGGATG
AGACTGGAACCCCAGTCCATGGTGTGGCTGCCCGTCCTGCACAGAGTGGCTGCTGCAGAAACTGCCAAGCATCAG
GCCAAATGTAACATCTGCAAAGAGTGTCCAATCATTGGATTCAGGTACAGGAGTCTAAAGCACTTTAATTATGAC
ATCTGCCAAAGCTGCTTTTTTTCTGGTCGAGTTGCAAAAGGCCATAAAATGCACTATCCCATGGTGGAATATTGC
ACTCCGACTACATCAGGAGAAGATGTTCGAGACTTTGCCAAGGTACTAAAAAACAAATTTCGAACCAAAAGGTAT
TTTGCGAAGCATCCCCGAATGGGCTACCTGCCAGTGCAGACTGTCTTAGAGGGGGACAACATGGAAACTCCCGTT
ACTCTGATCAACTTCTGGCCAGTAGATTCTGCGCCTGCCTCGTCCCCTCAGCTTTCACACGATGATACTCATTCA
CGCATTGAACATTATGCTAGCAGGCTAGCAGAAATGGAAAACAGCAATGGATCTTATCTAAATGATAGCATCTCT
CCTAATGAGAGCATAGATGATGAACATTTGTTAATCCAGCATTACTGCCAAAGTTTGAACCAGGACTCCCCCCTG
AGCCAGCCTCGTAGT
Human sequences with codon optimization:
(SEQ ID NO: 105)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCGCCGCTAGCGCCATGATGGTGATGACCGAGGACATGCCCCTGGAGATCAGCTACGTGCCCAGCACCTAC
CTGACCGAGATCACCCACGTGAGCCAGGCCCTGCTGGAGGTGGAGCAGCTGCTGAACGCCCCCGACCTGTGCGCC
AAGGACTTCGAGGACCTGTTCAAGCAGGAGGAGAGCCTGAAGAACATCAAGGACAGCCTGCAGCAGAGCAGCGGC
CGGATCGACATCATCCACAGCAAGAAGACCGCCGCCCTGCAGAGCGCCACCCCCGTGGAGCGGGTGAAGCTGCAG
GAGGCCCTGAGCCAGCTGGACTTCCAGTGGGAGAAGGTGAACAAGATGTACAAGGACCGGCAGGGCCGGTTCGAC
CGGAGCGTGGAGAAGTGGCGGCGGTTCCACTACGACATCAAGATCTTCAACCAGTGGCTGACCGAGGCCGAGCAG
TTCCTGCGGAAGACCCAGATCCCCGAGAACTGGGAGCACGCCAAGTACAAGTGGTACCTGAAGGAGCTGCAGGAC
GGCATCGGCCAGCGGCAGACCGTGGTGCGGACCCTGAACGCCACCGGCGAGGAGATCATCCAGCAGAGCAGCAAG
ACCGACGCCAGCATCCTGCAGGAGAAGCTGGGCAGCCTGAACCTGCGGTGGCAGGAGGTGTGCAAGCAGCTGAGC
GACCGGAAGAAGCGGCTGGAGGAGCAGAAGGGCCCCGCCAGCCAGCACTTCCTGAGCACCAGCGTGCAGGGCCCC
TGGGAGCGGGCCATCAGCCCCAACAAGGTGCCCTACTACATCAACCACGAGACCCAGACCACCTGCTGGGACCAC
CCCAAGATGACCGAGCTGTACCAGAGCCTGGCCGACCTGAACAACGTGCGGTTCAGCGCCTACCGGACCGCCATG
AAGCTGCGGCGGCTGCAGAAGGCCCTGTGCCTGGACCTGCTGAGCCTGAGCGCCGCCTGCGACGCCCTGGACCAG
CACAACCTGAAGCAGAACGACCAGCCCATGGACATCCTGCAGATCATCAACTGCCTGACCACCATCTACGACCGG
CTGGAGCAGGAGCACAACAACCTGGTGAACGTGCCCCTGTGCGTGGACATGTGCCTGAACTGGCTGCTGAACGTG
TACGACACCGGCCGGACCGGCCGGATCCGGGTGCTGAGCTTCAAGACCGGCATCATCAGCCTGTGCAAGGCCCAC
CTGGAGGACAAGTACCGGTACCTGTTCAAGCAGGTGGCCAGCAGCACCGGCTTCTGCGACCAGCGGCGGCTGGGC
CTGCTGCTGCACGACAGCATCCAGATCCCCCGGCAGCTGGGCGAGGTGGCCAGCTTCGGCGGCAGCAACATCGAG
CCCAGCGTGCGGAGCTGCTTCCAGTTCGCCAACAACAAGCCCGAGATCGAGGCCGCCCTGTTCCTGGACTGGATG
CGGCTGGAGCCCCAGAGCATGGTGTGGCTGCCCGTGCTGCACCGGGTGGCCGCCGCCGAGACCGCCAAGCACCAG
GCCAAGTGCAACATCTGCAAGGAGTGCCCCATCATCGGCTTCCGGTACCGGAGCCTGAAGCACTTCAACTACGAC
ATCTGCCAGAGCTGCTTCTTCAGCGGCCGGGTGGCCAAGGGCCACAAGATGCACTACCCCATGGTGGAGTACTGC
ACCCCCACCACCAGCGGCGAGGACGTGCGGGACTTCGCCAAGGTGCTGAAGAACAAGTTCCGGACCAAGCGGTAC
TTCGCCAAGCACCCCCGGATGGGCTACCTGCCCGTGCAGACCGTGCTGGAGGGCGACAACATGGAGACCCCCGTG
ACCCTGATCAACTTCTGGCCCGTGGACAGCGCCCCCGCCAGCAGCCCCCAGCTGAGCCACGACGACACCCACAGC
CGGATCGAGCACTACGCCAGCCGGCTGGCCGAGATGGAGAACAGCAACGGCAGCTACCTGAACGACAGCATCAGC
CCCAACGAGAGCATCGACGACGAGCACCTGCTGATCCAGCACTACTGCCAGAGCCTGAACCAGGACAGCCCCCTG
AGCCAGCCCCGGAGC
Utrophin N-terminus through R2_Dystrophin R16-R17_H4 to 1st coiled coil and
proline richregion that follows:
Human sequences without codon alterations:
(SEQ ID NO: 106)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCGCCGCTAGCGCCATGATGGTGATGACTGAAGACATGCCTTTGGAAATTTCTTATGTGCCTTCTACTTAT
TTGACTGAAATCACTCATGTCTCACAAGCCCTATTAGAAGTGGAACAACTTCTCAATGCTCCTGACCTCTGTGCT
AAGGACTTTGAAGATCTCTTTAAGCAAGAGGAGTCTCTGAAGAATATAAAAGATAGTCTACAACAAAGCTCAGGT
CGGATTGACATTATTCATAGCAAGAAGACAGCAGCATTGCAAAGTGCAACGCCTGTGGAAAGGGTGAAGCTACAG
GAAGCTCTCTCCCAGCTTGATTTCCAATGGGAAAAAGTTAACAAAATGTACAAGGACCGACAAGGGCGATTTGAC
AGATCTGTTGAGAAATGGCGGCGTTTTCATTATGATATAAAGATATTTAATCAGTGGCTAACAGAAGCTGAACAG
TTTCTCAGAAAGACACAAATTCCTGAGAATTGGGAACATGCTAAATACAAATGGTATCTTAAGGAACTCCAGGAT
GGCATTGGGCAGCGGCAAACTGTTGTCAGAACATTGAATGCAACTGGGGAAGAAATAATTCAGCAATCCTCAAAA
ACAGATGCCAGTATTCTACAGGAAAAATTGGGAAGCCTGAATCTGCGGTGGCAGGAGGTCTGCAAACAGCTGTCA
GACAGAAAAAAGAGGCTAGAAGAACAAAAGGGTCCAGCATCTCAGCACTTTCTTTCCACGTCTGTCCAGGGTCCC
TGGGAGAGAGCCATCTCGCCAAACAAAGTGCCCTACTATATCAACCACGAGACTCAAACAACTTGCTGGGACCAT
CCCAAAATGACAGAGCTCTACCAGTCTTTAGCTGACCTGAATAATGTCAGATTCTCAGCTTATAGGACTGCCATG
AAACTCCGAAGACTGCAGAAGGCCCTTTGCTTGGATCTCTTGAGCCTGTCAGCTGCATGTGATGCCTTGGACCAG
CACAACCTCAAGCAAAATGACCAGCCCATGGATATCCTGCAGATTATTAATTGTTTGACCACTATTTATGACCGC
CTGGAGCAAGAGCACAACAATTTGGTCAACGTCCCTCTCTGCGTGGATATGTGTCTGAACTGGCTGCTGAATGTT
TATGATACGGGACGAACAGGGAGGATCCGTGTCCTGTCTTTTAAAACTGGCATCATTTCCCTGTGTAAAGCACAT
TTGGAAGACAAGTACAGATACCTTTTCAAGCAAGTGGCAAGTTCAACAGGATTTTGTGACCAGCGCAGGCTGGGC
CTCCTTCTGCATGATTCTATCCAAATTCCAAGACAGTTGGGTGAAGTTGCATCCTTTGGGGGCAGTAACATTGAG
CCAAGTGTCCGGAGCTGCTTCCAATTTGCTAATAATAAGCCAGAGATCGAAGCGGCCCTCTTCCTAGACTGGATG
AGACTGGAACCCCAGTCCATGGTGTGGCTGCCCGTCCTGCACAGAGTGGCTGCTGCAGAAACTGCCAAGCATCAG
GCCAAATGTAACATCTGCAAAGAGTGTCCAATCATTGGATTCAGGTACAGGAGTCTAAAGCACTTTAATTATGAC
ATCTGCCAAAGCTGCTTTTTTTCTGGTCGAGTTGCAAAAGGCCATAAAATGCACTATCCCATGGTGGAATATTGC
ACTCCGACTACATCAGGAGAAGATGTTCGAGACTTTGCCAAGGTACTAAAAAACAAATTTCGAACCAAAAGGTAT
TTTGCGAAGCATCCCCGAATGGGCTACCTGCCAGTGCAGACTGTCTTAGAGGGGGACAACATGGAAACTCCCGTT
ACTCTGATCAACTTCTGGCCAGTAGATTCTGCGCCTGCCTCGTCCCCTCAGCTTTCACACGATGATACTCATTCA
CGCATTGAACATTATGCTAGCAGGCTAGCAGAAATGGAAAACAGCAATGGATCTTATCTAAATGATAGCATCTCT
CCTAATGAGAGCATAGATGATGAACATTTGTTAATCCAGCATTACTGCCAAAGTTTGAACCAGGACTCCCCCCTG
AGCCAGCCTCGTAGTCCTGCCCAGATCTTGATTTCCTTAGAGAGTGAGGAAAGAGGGGAGCTAGAGAGAATCCTA
GCAGATCTTGAGGAAGAAAACAGGAATCTGCAAGCAGAATATGACCGTCTAAAGCAGCAGCACGAACATAAAGGC
CTGTCCCCACTGCCGTCCCCTCCTGAAATGATGCCCACCTCTCCCCAGAGTCCC
Human sequences with codon optimization:
(SEQ ID NO: 107)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCGCCGCTAGCGCCATGATGGTGATGACCGAGGACATGCCCCTGGAGATCAGCTACGTGCCCAGCACCTAC
CTGACCGAGATCACCCACGTGAGCCAGGCCCTGCTGGAGGTGGAGCAGCTGCTGAACGCCCCCGACCTGTGCGCC
AAGGACTTCGAGGACCTGTTCAAGCAGGAGGAGAGCCTGAAGAACATCAAGGACAGCCTGCAGCAGAGCAGCGGC
CGGATCGACATCATCCACAGCAAGAAGACCGCCGCCCTGCAGAGCGCCACCCCCGTGGAGCGGGTGAAGCTGCAG
GAGGCCCTGAGCCAGCTGGACTTCCAGTGGGAGAAGGTGAACAAGATGTACAAGGACCGGCAGGGCCGGTTCGAC
CGGAGCGTGGAGAAGTGGCGGCGGTTCCACTACGACATCAAGATCTTCAACCAGTGGCTGACCGAGGCCGAGCAG
TTCCTGCGGAAGACCCAGATCCCCGAGAACTGGGAGCACGCCAAGTACAAGTGGTACCTGAAGGAGCTGCAGGAC
GGCATCGGCCAGCGGCAGACCGTGGTGCGGACCCTGAACGCCACCGGCGAGGAGATCATCCAGCAGAGCAGCAAG
ACCGACGCCAGCATCCTGCAGGAGAAGCTGGGCAGCCTGAACCTGCGGTGGCAGGAGGTGTGCAAGCAGCTGAGC
GACCGGAAGAAGCGGCTGGAGGAGCAGAAGGGCCCCGCCAGCCAGCACTTCCTGAGCACCAGCGTGCAGGGCCCC
TGGGAGCGGGCCATCAGCCCCAACAAGGTGCCCTACTACATCAACCACGAGACCCAGACCACCTGCTGGGACCAC
CCCAAGATGACCGAGCTGTACCAGAGCCTGGCCGACCTGAACAACGTGCGGTTCAGCGCCTACCGGACCGCCATG
AAGCTGCGGCGGCTGCAGAAGGCCCTGTGCCTGGACCTGCTGAGCCTGAGCGCCGCCTGCGACGCCCTGGACCAG
CACAACCTGAAGCAGAACGACCAGCCCATGGACATCCTGCAGATCATCAACTGCCTGACCACCATCTACGACCGG
CTGGAGCAGGAGCACAACAACCTGGTGAACGTGCCCCTGTGCGTGGACATGTGCCTGAACTGGCTGCTGAACGTG
TACGACACCGGCCGGACCGGCCGGATCCGGGTGCTGAGCTTCAAGACCGGCATCATCAGCCTGTGCAAGGCCCAC
CTGGAGGACAAGTACCGGTACCTGTTCAAGCAGGTGGCCAGCAGCACCGGCTTCTGCGACCAGCGGCGGCTGGGC
CTGCTGCTGCACGACAGCATCCAGATCCCCCGGCAGCTGGGCGAGGTGGCCAGCTTCGGCGGCAGCAACATCGAG
CCCAGCGTGCGGAGCTGCTTCCAGTTCGCCAACAACAAGCCCGAGATCGAGGCCGCCCTGTTCCTGGACTGGATG
CGGCTGGAGCCCCAGAGCATGGTGTGGCTGCCCGTGCTGCACCGGGTGGCCGCCGCCGAGACCGCCAAGCACCAG
GCCAAGTGCAACATCTGCAAGGAGTGCCCCATCATCGGCTTCCGGTACCGGAGCCTGAAGCACTTCAACTACGAC
ATCTGCCAGAGCTGCTTCTTCAGCGGCCGGGTGGCCAAGGGCCACAAGATGCACTACCCCATGGTGGAGTACTGC
ACCCCCACCACCAGCGGCGAGGACGTGCGGGACTTCGCCAAGGTGCTGAAGAACAAGTTCCGGACCAAGCGGTAC
TTCGCCAAGCACCCCCGGATGGGCTACCTGCCCGTGCAGACCGTGCTGGAGGGCGACAACATGGAGACCCCCGTG
ACCCTGATCAACTTCTGGCCCGTGGACAGCGCCCCCGCCAGCAGCCCCCAGCTGAGCCACGACGACACCCACAGC
CGGATCGAGCACTACGCCAGCCGGCTGGCCGAGATGGAGAACAGCAACGGCAGCTACCTGAACGACAGCATCAGC
CCCAACGAGAGCATCGACGACGAGCACCTGCTGATCCAGCACTACTGCCAGAGCCTGAACCAGGACAGCCCCCTG
AGCCAGCCCCGGAGCCCCGCCCAGATCCTGATCAGCCTGGAGAGCGAGGAGCGGGGCGAGCTGGAGCGGATCCTG
GCCGACCTGGAGGAGGAGAACCGGAACCTGCAGGCCGAGTACGACCGGCTGAAGCAGCAGCACGAGCACAAGGGC
CTGAGCCCCCTGCCCAGCCCCCCCGAGATGATGCCCACCAGCCCCCAGAGCCCC
Utrophin N-terminus through R1_Dystrophin R16-R17_R23 to end of syntrophin region:
Human sequences without codon alterations:
(SEQ ID NO: 108)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGCCGCTAGCGCCATG
ATGGTGATGACTGAAGACATGCCTTTGGAAATTTCTTATGTGCCTTCTACTTATTTGACTGAAATCACTCATGTC
TCACAAGCCCTATTAGAAGTGGAACAACTTCTCAATGCTCCTGACCTCTGTGCTAAGGACTTTGAAGATCTCTTT
AAGCAAGAGGAGTCTCTGAAGAATATAAAAGATAGTCTACAACAAAGCTCAGGTCGGATTGACATTATTCATAGC
AAGAAGACAGCAGCATTGCAAAGTGCAACGCCTGTGGAAAGGGTGAAGCTACAGGAAGCTCTCTCCCAGCTTGAT
TTCCAATGGGAAAAAGTTAACAAAATGTACAAGGACCGACAAGGGCGATTTGACAGATCTGTTGAGAAATGGCGG
CGTTTTCATTATGATATAAAGATATTTAATCAGTGGCTAACAGAAGCTGAACAGTTTCTCAGAAAGACACAAATT
CCTGAGAATTGGGAACATGCTAAATACAAATGGTATCTTAAGGAACTCCAGGATGGCATTGGGCAGCGGCAAACT
GTTGTCAGAACATTGAATGCAACTGGGGAAGAAATAATTCAGCAATCCTCAAAAACAGATGCCAGTATTCTACAG
GAAAAATTGGGAAGCCTGAATCTGCGGTGGCAGGAGGTCTGCAAACAGCTGTCAGACAGAAAAAAGAGGCTAGAA
GAACAAAAGAGTTCTGACCAGTGGAAGCGTCTGCACCTTTCTCTGCAGGAACTTCTGGTGTGGCTACAGCTGAAA
GATGATGAATTAAGCCGGCAGGCACCTATTGGAGGCGACTTTCCAGCAGTTCAGAAGCAGAACGATGTACATAGG
GCCTTCAAGAGGGAATTGAAAACTAAAGAACCTGTAATCATGAGTACTCTTGAGACTGTACGAATATTTCTGACA
GAGCAGCCTTTGGAAGGACTAGAGAAACTCTACCAGGAGCCCAGAGAGCTGCCTCCTGAGGAGAGAGCCCAGAAT
GTCACTCGGCTTCTACGAAAGCAGGCTGAGGAGGTCAATACTGAGTGGGAAAAATTGAACCTGCACTCCGCTGAC
TGGCAGAGAAAAATAGATGAGACCCTTGAAAGACTCCGGGAACTTCAAGAGGCCACGGATGAGCTGGACCTCAAG
CTGCGCCAAGCTGAGGTGATCAAGGGATCCTGGCAGCCCGTGGGCGATCTCCTCATTGACTCTCTCCAAGATCAC
CTCGAGAAAGTCAAGGCACTTCGAGGAGAAATTGCGCCTCTGAAAGAGAACGTGAGCCACGTCAATGACCTTGCT
CGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTTGGTCCAGCATCT
CAGCACTTTCTTTCCACGTCTGTCCAGGGTCCCTGGGAGAGAGCCATCTCGCCAAACAAAGTGCCCTACTATATC
AACCACGAGACTCAAACAACTTGCTGGGACCATCCCAAAATGACAGAGCTCTACCAGTCTTTAGCTGACCTGAAT
AATGTCAGATTCTCAGCTTATAGGACTGCCATGAAACTCCGAAGACTGCAGAAGGCCCTTTGCTTGGATCTCTTG
AGCCTGTCAGCTGCATGTGATGCCTTGGACCAGCACAACCTCAAGCAAAATGACCAGCCCATGGATATCCTGCAG
ATTATTAATTGTTTGACCACTATTTATGACCGCCTGGAGCAAGAGCACAACAATTTGGTCAACGTCCCTCTCTGC
GTGGATATGTGTCTGAACTGGCTGCTGAATGTTTATGATACGGGACGAACAGGGAGGATCCGTGTCCTGTCTTTT
AAAACTGGCATCATTTCCCTGTGTAAAGCACATTTGGAAGACAAGTACAGATACCTTTTCAAGCAAGTGGCAAGT
TCAACAGGATTTTGTGACCAGCGCAGGCTGGGCCTCCTTCTGCATGATTCTATCCAAATTCCAAGACAGTTGGGT
GAAGTTGCATCCTTTGGGGGCAGTAACATTGAGCCAAGTGTCCGGAGCTGCTTCCAATTTGCTAATAATAAGCCA
GAGATCGAAGCGGCCCTCTTCCTAGACTGGATGAGACTGGAACCCCAGTCCATGGTGTGGCTGCCCGTCCTGCAC
AGAGTGGCTGCTGCAGAAACTGCCAAGCATCAGGCCAAATGTAACATCTGCAAAGAGTGTCCAATCATTGGATTC
AGGTACAGGAGTCTAAAGCACTTTAATTATGACATCTGCCAAAGCTGCTTTTTTTCTGGTCGAGTTGCAAAAGGC
CATAAAATGCACTATCCCATGGTGGAATATTGCACTCCGACTACATCAGGAGAAGATGTTCGAGACTTTGCCAAG
GTACTAAAAAACAAATTTCGAACCAAAAGGTATTTTGCGAAGCATCCCCGAATGGGCTACCTGCCAGTGCAGACT
GTCTTAGAGGGGGACAACATGGAAACTCCCGTTACTCTGATCAACTTCTGGCCAGTAGATTCTGCGCCTGCCTCG
TCCCCTCAGCTTTCACACGATGATACTCATTCACGCATTGAACATTATGCTAGCAGGCTAGCAGAAATGGAAAAC
AGCAATGGATCTTATCTAAATGATAGCATCTCTCCTAATGAGAGCATAGATGATGAACATTTGTTAATCCAGCAT
TACTGCCAAAGTTTGAACCAGGACTCCCCCCTGAGCCAGCCTCGTAGT
Human sequences with codon optimization:
(SEQ ID NO: 109)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGCCGCTAGCGCCATG
ATGGTGATGACCGAGGACATGCCCCTGGAGATCAGCTACGTGCCCAGCACCTACCTGACCGAGATCACCCACGTG
AGCCAGGCCCTGCTGGAGGTGGAGCAGCTGCTGAACGCCCCCGACCTGTGCGCCAAGGACTTCGAGGACCTGTTC
AAGCAGGAGGAGAGCCTGAAGAACATCAAGGACAGCCTGCAGCAGAGCAGCGGCCGGATCGACATCATCCACAGC
AAGAAGACCGCCGCCCTGCAGAGCGCCACCCCCGTGGAGCGGGTGAAGCTGCAGGAGGCCCTGAGCCAGCTGGAC
TTCCAGTGGGAGAAGGTGAACAAGATGTACAAGGACCGGCAGGGCCGGTTCGACCGGAGCGTGGAGAAGTGGCGG
CGGTTCCACTACGACATCAAGATCTTCAACCAGTGGCTGACCGAGGCCGAGCAGTTCCTGCGGAAGACCCAGATC
CCCGAGAACTGGGAGCACGCCAAGTACAAGTGGTACCTGAAGGAGCTGCAGGACGGCATCGGCCAGCGGCAGACC
GTGGTGCGGACCCTGAACGCCACCGGCGAGGAGATCATCCAGCAGAGCAGCAAGACCGACGCCAGCATCCTGCAG
GAGAAGCTGGGCAGCCTGAACCTGCGGTGGCAGGAGGTGTGCAAGCAGCTGAGCGACCGGAAGAAGCGGCTGGAG
GAGCAGAAGAGCAGCGACCAGTGGAAGCGGCTGCACCTGAGCCTGCAGGAGCTGCTGGTGTGGCTGCAGCTGAAG
GACGACGAGCTGAGCCGGCAGGCCCCCATCGGCGGCGACTTCCCCGCCGTGCAGAAGCAGAACGACGTGCACCGG
GCCTTCAAGCGGGAGCTGAAGACCAAGGAGCCCGTGATCATGAGCACCCTGGAGACCGTGCGGATCTTCCTGACC
GAGCAGCCCCTGGAGGGCCTGGAGAAGCTGTACCAGGAGCCCCGGGAGCTGCCCCCCGAGGAGCGGGCCCAGAAC
GTGACCCGGCTGCTGCGGAAGCAGGCCGAGGAGGTGAACACCGAGTGGGAGAAGCTGAACCTGCACAGCGCCGAC
TGGCAGCGGAAGATCGACGAGACCCTGGAGCGGCTGCAGGAGCTGCAGGAGGCCACCGACGAGCTGGACCTGAAG
CTGCGGCAGGCCGAGGTGATCAAGGGCAGCTGGCAGCCCGTGGGCGACCTGCTGATCGACAGCCTGCAGGACCAC
CTGGAGAAGGTGAAGGCCCTGCGGGGCGAGATCGCCCCCCTGAAGGAGAACGTGAGCCACGTGAACGACCTGGCC
CGGCAGCTGACCACCCTGGGCATCCAGCTGAGCCCCTACAACCTGAGCACCCTGGAGGACCTGAACACCCGGTGG
AAGCTGCTGCAGGTGGCCGTGGAGGACCGGGTGCGGCAGCTGCACGAGGCCCACCGGGACTTCGGCCCCGCCAGC
CAGCACTTCCTGAGCACCAGCGTGCAGGGCCCCTGGGAGCGGGCCATCAGCCCCAACAAGGTGCCCTACTACATC
AACCACGAGACCCAGACCACCTGCTGGGACCACCCCAAGATGACCGAGCTGTACCAGAGCCTGGCCGACCTGAAC
AACGTGCGGTTCAGCGCCTACCGGACCGCCATGAAGCTGCGGCGGCTGCAGAAGGCCCTGTGCCTGGACCTGCTG
AGCCTGAGCGCCGCCTGCGACGCCCTGGACCAGCACAACCTGAAGCAGAACGACCAGCCCATGGACATCCTGCAG
ATCATCAACTGCCTGACCACCATCTACGACCGGCTGGAGCAGGAGCACAACAACCTGGTGAACGTGCCCCTGTGC
GTGGACATGTGCCTGAACTGGCTGCTGAACGTGTACGACACCGGCCGGACCGGCCGGATCCGGGTGCTGAGCTTC
AAGACCGGCATCATCAGCCTGTGCAAGGCCCACCTGGAGGACAAGTACCGGTACCTGTTCAAGCAGGTGGCCAGC
AGCACCGGCTTCTGCGACCAGCGGCGGCTGGGCCTGCTGCTGCACGACAGCATCCAGATCCCCCGGCAGCTGGGC
GAGGTGGCCAGCTTCGGCGGCAGCAACATCGAGCCCAGCGTGCGGAGCTGCTTCCAGTTCGCCAACAACAAGCCC
GAGATCGAGGCCGCCCTGTTCCTGGACTGGATGCGGCTGGAGCCCCAGAGCATGGTGTGGCTGCCCGTGCTGCAC
CGGGTGGCCGCCGCCGAGACCGCCAAGCACCAGGCCAAGTGCAACATCTGCAAGGAGTGCCCCATCATCGGCTTC
CGGTACCGGAGCCTGAAGCACTTCAACTACGACATCTGCCAGAGCTGCTTCTTCAGCGGCCGGGTGGCCAAGGGC
CACAAGATGCACTACCCCATGGTGGAGTACTGCACCCCCACCACCAGCGGCGAGGACGTGCGGGACTTCGCCAAG
GTGCTGAAGAACAAGTTCCGGACCAAGCGGTACTTCGCCAAGCACCCCCGGATGGGCTACCTGCCCGTGCAGACC
GTGCTGGAGGGCGACAACATGGAGACCCCCGTGACCCTGATCAACTTCTGGCCCGTGGACAGCGCCCCCGCCAGC
AGCCCCCAGCTGAGCCACGACGACACCCACAGCCGGATCGAGCACTACGCCAGCCGGCTGGCCGAGATGGAGAAC
AGCAACGGCAGCTACCTGAACGACAGCATCAGCCCCAACGAGAGCATCGACGACGAGCACCTGCTGATCCAGCAC
TACTGCCAGAGCCTGAACCAGGACAGCCCCCTGAGCCAGCCCCGGAGC
Utrophin N-terminus through R2_Dystrophin R16-R17_R24 + H4 to end of syntrophin
region:
Human sequences without codon alterations:
(SEQ ID NO: 110)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCGCCGCTAGCGCCATGATGGTGATGACTGAAGACATGCCTTTGGAAATTTCTTATGTGCCTTCTACTTAT
TTGACTGAAATCACTCATGTCTCACAAGCCCTATTAGAAGTGGAACAACTTCTCAATGCTCCTGACCTCTGTGCT
AAGGACTTTGAAGATCTCTTTAAGCAAGAGGAGTCTCTGAAGAATATAAAAGATAGTCTACAACAAAGCTCAGGT
CGGATTGACATTATTCATAGCAAGAAGACAGCAGCATTGCAAAGTGCAACGCCTGTGGAAAGGGTGAAGCTACAG
GAAGCTCTCTCCCAGCTTGATTTCCAATGGGAAAAAGTTAACAAAATGTACAAGGACCGACAAGGGCGATTTGAC
AGATCTGTTGAGAAATGGCGGCGTTTTCATTATGATATAAAGATATTTAATCAGTGGCTAACAGAAGCTGAACAG
TTTCTCAGAAAGACACAAATTCCTGAGAATTGGGAACATGCTAAATACAAATGGTATCTTAAGGAACTCCAGGAT
GGCATTGGGCAGCGGCAAACTGTTGTCAGAACATTGAATGCAACTGGGGAAGAAATAATTCAGCAATCCTCAAAA
ACAGATGCCAGTATTCTACAGGAAAAATTGGGAAGCCTGAATCTGCGGTGGCAGGAGGTCTGCAAACAGCTGTCA
GACAGAAAAAAGAGGCTAGAAGAACAAAAGTGGCAGAGAAAAATAGATGAGACCCTTGAAAGACTCCGGGAACTT
CAAGAGGCCACGGATGAGCTGGACCTCAAGCTGCGCCAAGCTGAGGTGATCAAGGGATCCTGGCAGCCCGTGGGC
GATCTCCTCATTGACTCTCTCCAAGATCACCTCGAGAAAGTCAAGGCACTTCGAGGAGAAATTGCGCCTCTGAAA
GAGAACGTGAGCCACGTCAATGACCTTGCTCGCCAGCTTACCACTTTGGGCATTCAGCTCTCACCGTATAACCTC
AGCACTCTGGAAGACCTGAACACCAGATGGAAGCTTCTGCAGGTGGCCGTCGAGGACCGAGTCAGGCAGCTGCAT
GAAGCCCACAGGGACTTTGGTCCAGCATCTCAGCACTTTCTTTCCACGTCTGTCCAGGGTCCCTGGGAGAGAGCC
ATCTCGCCAAACAAAGTGCCCTACTATATCAACCACGAGACTCAAACAACTTGCTGGGACCATCCCAAAATGACA
GAGCTCTACCAGTCTTTAGCTGACCTGAATAATGTCAGATTCTCAGCTTATAGGACTGCCATGAAACTCCGAAGA
CTGCAGAAGGCCCTTTGCTTGGATCTCTTGAGCCTGTCAGCTGCATGTGATGCCTTGGACCAGCACAACCTCAAG
CAAAATGACCAGCCCATGGATATCCTGCAGATTATTAATTGTTTGACCACTATTTATGACCGCCTGGAGCAAGAG
CACAACAATTTGGTCAACGTCCCTCTCTGCGTGGATATGTGTCTGAACTGGCTGCTGAATGTTTATGATACGGGA
CGAACAGGGAGGATCCGTGTCCTGTCTTTTAAAACTGGCATCATTTCCCTGTGTAAAGCACATTTGGAAGACAAG
TACAGATACCTTTTCAAGCAAGTGGCAAGTTCAACAGGATTTTGTGACCAGCGCAGGCTGGGCCTCCTTCTGCAT
GATTCTATCCAAATTCCAAGACAGTTGGGTGAAGTTGCATCCTTTGGGGGCAGTAACATTGAGCCAAGTGTCCGG
AGCTGCTTCCAATTTGCTAATAATAAGCCAGAGATCGAAGCGGCCCTCTTCCTAGACTGGATGAGACTGGAACCC
CAGTCCATGGTGTGGCTGCCCGTCCTGCACAGAGTGGCTGCTGCAGAAACTGCCAAGCATCAGGCCAAATGTAAC
ATCTGCAAAGAGTGTCCAATCATTGGATTCAGGTACAGGAGTCTAAAGCACTTTAATTATGACATCTGCCAAAGC
TGCTTTTTTTCTGGTCGAGTTGCAAAAGGCCATAAAATGCACTATCCCATGGTGGAATATTGCACTCCGACTACA
TCAGGAGAAGATGTTCGAGACTTTGCCAAGGTACTAAAAAACAAATTTCGAACCAAAAGGTATTTTGCGAAGCAT
CCCCGAATGGGCTACCTGCCAGTGCAGACTGTCTTAGAGGGGGACAACATGGAAACTCCCGTTACTCTGATCAAC
TTCTGGCCAGTAGATTCTGCGCCTGCCTCGTCCCCTCAGCTTTCACACGATGATACTCATTCACGCATTGAACAT
TATGCTAGCAGGCTAGCAGAAATGGAAAACAGCAATGGATCTTATCTAAATGATAGCATCTCTCCTAATGAGAGC
ATAGATGATGAACATTTGTTAATCCAGCATTACTGCCAAAGTTTGAACCAGGACTCCCCCCTGAGCCAGCCTCGT
AGT
Human sequences with codon optimization:
(SEQ ID NO: 111)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCGCCGCTAGCGCCATGATGGTGATGACCGAGGACATGCCCCTGGAGATCAGCTACGTGCCCAGCACCTAC
CTGACCGAGATCACCCACGTGAGCCAGGCCCTGCTGGAGGTGGAGCAGCTGCTGAACGCCCCCGACCTGTGCGCC
AAGGACTTCGAGGACCTGTTCAAGCAGGAGGAGAGCCTGAAGAACATCAAGGACAGCCTGCAGCAGAGCAGCGGC
CGGATCGACATCATCCACAGCAAGAAGACCGCCGCCCTGCAGAGCGCCACCCCCGTGGAGCGGGTGAAGCTGCAG
GAGGCCCTGAGCCAGCTGGACTTCCAGTGGGAGAAGGTGAACAAGATGTACAAGGACCGGCAGGGCCGGTTCGAC
CGGAGCGTGGAGAAGTGGCGGCGGTTCCACTACGACATCAAGATCTTCAACCAGTGGCTGACCGAGGCCGAGCAG
TTCCTGCGGAAGACCCAGATCCCCGAGAACTGGGAGCACGCCAAGTACAAGTGGTACCTGAAGGAGCTGCAGGAC
GGCATCGGCCAGCGGCAGACCGTGGTGCGGACCCTGAACGCCACCGGCGAGGAGATCATCCAGCAGAGCAGCAAG
ACCGACGCCAGCATCCTGCAGGAGAAGCTGGGCAGCCTGAACCTGCGGTGGCAGGAGGTGTGCAAGCAGCTGAGC
GACCGGAAGAAGCGGCTGGAGGAGCAGAAGTGGCAGCGGAAGATCGACGAGACCCTGGAGCGGCTGCAGGAGCTG
CAGGAGGCCACCGACGAGCTGGACCTGAAGCTGCGGCAGGCCGAGGTGATCAAGGGCAGCTGGCAGCCCGTGGGC
GACCTGCTGATCGACAGCCTGCAGGACCACCTGGAGAAGGTGAAGGCCCTGCGGGGCGAGATCGCCCCCCTGAAG
GAGAACGTGAGCCACGTGAACGACCTGGCCCGGCAGCTGACCACCCTGGGCATCCAGCTGAGCCCCTACAACCTG
AGCACCCTGGAGGACCTGAACACCCGGTGGAAGCTGCTGCAGGTGGCCGTGGAGGACCGGGTGCGGCAGCTGCAC
GAGGCCCACCGGGACTTCGGCCCCGCCAGCCAGCACTTCCTGAGCACCAGCGTGCAGGGCCCCTGGGAGCGGGCC
ATCAGCCCCAACAAGGTGCCCTACTACATCAACCACGAGACCCAGACCACCTGCTGGGACCACCCCAAGATGACC
GAGCTGTACCAGAGCCTGGCCGACCTGAACAACGTGCGGTTCAGCGCCTACCGGACCGCCATGAAGCTGCGGCGG
CTGCAGAAGGCCCTGTGCCTGGACCTGCTGAGCCTGAGCGCCGCCTGCGACGCCCTGGACCAGCACAACCTGAAG
CAGAACGACCAGCCCATGGACATCCTGCAGATCATCAACTGCCTGACCACCATCTACGACCGGCTGGAGCAGGAG
CACAACAACCTGGTGAACGTGCCCCTGTGCGTGGACATGTGCCTGAACTGGCTGCTGAACGTGTACGACACCGGC
CGGACCGGCCGGATCCGGGTGCTGAGCTTCAAGACCGGCATCATCAGCCTGTGCAAGGCCCACCTGGAGGACAAG
TACCGGTACCTGTTCAAGCAGGTGGCCAGCAGCACCGGCTTCTGCGACCAGCGGCGGCTGGGCCTGCTGCTGCAC
GACAGCATCCAGATCCCCCGGCAGCTGGGCGAGGTGGCCAGCTTCGGCGGCAGCAACATCGAGCCCAGCGTGCGG
AGCTGCTTCCAGTTCGCCAACAACAAGCCCGAGATCGAGGCCGCCCTGTTCCTGGACTGGATGCGGCTGGAGCCC
CAGAGCATGGTGTGGCTGCCCGTGCTGCACCGGGTGGCCGCCGCCGAGACCGCCAAGCACCAGGCCAAGTGCAAC
ATCTGCAAGGAGTGCCCCATCATCGGCTTCCGGTACCGGAGCCTGAAGCACTTCAACTACGACATCTGCCAGAGC
TGCTTCTTCAGCGGCCGGGTGGCCAAGGGCCACAAGATGCACTACCCCATGGTGGAGTACTGCACCCCCACCACC
AGCGGCGAGGACGTGCGGGACTTCGCCAAGGTGCTGAAGAACAAGTTCCGGACCAAGCGGTACTTCGCCAAGCAC
CCCCGGATGGGCTACCTGCCCGTGCAGACCGTGCTGGAGGGCGACAACATGGAGACCCCCGTGACCCTGATCAAC
TTCTGGCCCGTGGACAGCGCCCCCGCCAGCAGCCCCCAGCTGAGCCACGACGACACCCACAGCCGGATCGAGCAC
TACGCCAGCCGGCTGGCCGAGATGGAGAACAGCAACGGCAGCTACCTGAACGACAGCATCAGCCCCAACGAGAGC
ATCGACGACGAGCACCTGCTGATCCAGCACTACTGCCAGAGCCTGAACCAGGACAGCCCCCTGAGCCAGCCCCGG
AGC
Utrophin N-terminus through R2_Dystrophin R23 + R24 + H4 to end of syntrophin
region:
Human sequences without codon alterations:
(SEQ ID NO: 112)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCGCCGCTAGCGCCAGTTCTGACCAGTGGAAGCGTCTGCACCTTTCTCTGCAGGAACTTCTGGTGTGGCTA
CAGCTGAAAGATGATGAATTAAGCCGGCAGGCACCTATTGGAGGCGACTTTCCAGCAGTTCAGAAGCAGAACGAT
GTACATAGGGCCTTCAAGAGGGAATTGAAAACTAAAGAACCTGTAATCATGAGTACTCTTGAGACTGTACGAATA
TTTCTGACAGAGCAGCCTTTGGAAGGACTAGAGAAACTCTACCAGGAGCCCAGAGAGCTGCCTCCTGAGGAGAGA
GCCCAGAATGTCACTCGGCTTCTACGAAAGCAGGCTGAGGAGGTCAATACTGAGTGGGAAAAATTGAACCTGCAC
TCCGCTGACTGGCAGAGAAAAATAGATGAGACCCTTGAAAGACTCCGGGAACTTCAAGAGGCCACGGATGAGCTG
GACCTCAAGCTGCGCCAAGCTGAGGTGATCAAGGGATCCTGGCAGCCCGTGGGCGATCTCCTCATTGACTCTCTC
CAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAAC
ACCAGATGGAAGCTTCTGCAGGTGGCCGTCGAGGACCGAGTCAGGCAGCTGCATGAAGCCCACAGGGACTTTGGT
CCAGCATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTTTAGCT
GACCTGAATAATGTCAGATTCTCAGCTTATAGGACTGCCATGAAACTCCGAAGACTGCAGAAGGCCCTTTGCTTG
GATCTCTTGAGCCTGTCAGCTGCATGTGATGCCTTGGACCAGCACAACCTCAAGCAAAATGACCAGCCCATGGAT
ATCCTGCAGATTATTAATTGTTTGACCACTATTTATGACCGCCTGGAGCAAGAGCACAACAATTTGGTCAACGTC
CCTCTCTGCGTGGATATGTGTCTGAACTGGCTGCTGAATGTTTATGATACGGGACGAACAGGGAGGATCCGTGTC
CTGTCTTTTAAAACTGGCATCATTTCCCTGTGTAAAGCACATTTGGAAGACAAGTACAGATACCTTTTCAAGCAA
GTGGCAAGTTCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCC
GTCCTGCACAGAGTGGCTGCTGCAGAAACTGCCAAGCATCAGGCCAAATGTAACATCTGCAAAGAGTGTCCAATC
ATTGGATTCAGGTACAGGAGTCTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTACATCAGGAGAAGATGTTCGAGAC
TTTGCCAAGGTACTAAAAAACAAATTTCGAACCAAAAGGTATTTTGCGAAGCATCCCCGAATGGGCTACCTGCCA
GTGCAGACTGTCTTAGAGGGGGACAACATGGAAACTCCCGTTACTCTGATCAACTTCTGGCCAGTAGATTCTGCG
CCTGCCTCGTCCCCTCAGCTTTCACACGATGATACTCATTCACGCATTGAACATTATGCTAGCAGGCTAGCAGAA
ATGGAAAACAGCAATGGATCTTATCTAAATGATAGCATCTCTCCTAATGAGAGCATAGATGATGAACATTTGTTA
ATCCAGCATTACTGCCAAAGTTTGAACCAGGACTCCCCCCTGAGCCAGCCTCGTAGT
Human sequences with codon optimization:
(SEQ ID NO: 113)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCGCCGCTAGCGCCAGCAGCGACCAGTGGAAGCGGCTGCACCTGAGCCTGCAGGAGCTGCTGGTGTGGCTG
CAGCTGAAGGACGACGAGCTGAGCCGGCAGGCCCCCATCGGCGGCGACTTCCCCGCCGTGCAGAAGCAGAACGAC
GTGCACCGGGCCTTCAAGCGGGAGCTGAAGACCAAGGAGCCCGTGATCATGAGCACCCTGGAGACCGTGCGGATC
TTCCTGACCGAGCAGCCCCTGGAGGGCCTGGAGAAGCTGTACCAGGAGCCCCGGGAGCTGCCCCCCGAGGAGCGG
GCCCAGAACGTGACCCGGCTGCTGCGGAAGCAGGCCGAGGAGGTGAACACCGAGTGGGAGAAGCTGAACCTGCAC
AGCGCCGACTGGCAGCGGAAGATCGACGAGACCCTGGAGCGGCTGCAGGAGCTGCAGGAGGCCACCGACGAGCTG
GACCTGAAGCTGCGGCAGGCCGAGGTGATCAAGGGCAGCTGGCAGCCCGTGGGCGACCTGCTGATCGACAGCCTG
CAGGACCACCTGGAGAAGGTGAAGGCCCTGCGGGGCGAGATCGCCCCCCTGAAGGAGAACGTGAGCCACGTGAAC
GACCTGGCCCGGCAGCTGACCACCCTGGGCATCCAGCTGAGCCCCTACAACCTGAGCACCCTGGAGGACCTGAAC
ACCCGGTGGAAGCTGCTGCAGGTGGCCGTGGAGGACCGGGTGCGGCAGCTGCACGAGGCCCACCGGGACTTCGGC
CCCGCCAGCCAGCACTTCCTGAGCACCAGCGTGCAGGGCCCCTGGGAGCGGGCCATCAGCCCCAACAAGGTGCCC
TACTACATCAACCACGAGACCCAGACCACCTGCTGGGACCACCCCAAGATGACCGAGCTGTACCAGAGCCTGGCC
GACCTGAACAACGTGCGGTTCAGCGCCTACCGGACCGCCATGAAGCTGCGGCGGCTGCAGAAGGCCCTGTGCCTG
GACCTGCTGAGCCTGAGCGCCGCCTGCGACGCCCTGGACCAGCACAACCTGAAGCAGAACGACCAGCCCATGGAC
ATCCTGCAGATCATCAACTGCCTGACCACCATCTACGACCGGCTGGAGCAGGAGCACAACAACCTGGTGAACGTG
CCCCTGTGCGTGGACATGTGCCTGAACTGGCTGCTGAACGTGTACGACACCGGCCGGACCGGCCGGATCCGGGTG
CTGAGCTTCAAGACCGGCATCATCAGCCTGTGCAAGGCCCACCTGGAGGACAAGTACCGGTACCTGTTCAAGCAG
GTGGCCAGCAGCACCGGCTTCTGCGACCAGCGGCGGCTGGGCCTGCTGCTGCACGACAGCATCCAGATCCCCCGG
CAGCTGGGCGAGGTGGCCAGCTTCGGCGGCAGCAACATCGAGCCCAGCGTGCGGAGCTGCTTCCAGTTCGCCAAC
AACAAGCCCGAGATCGAGGCCGCCCTGTTCCTGGACTGGATGCGGCTGGAGCCCCAGAGCATGGTGTGGCTGCCC
GTGCTGCACCGGGTGGCCGCCGCCGAGACCGCCAAGCACCAGGCCAAGTGCAACATCTGCAAGGAGTGCCCCATC
ATCGGCTTCCGGTACCGGAGCCTGAAGCACTTCAACTACGACATCTGCCAGAGCTGCTTCTTCAGCGGCCGGGTG
GCCAAGGGCCACAAGATGCACTACCCCATGGTGGAGTACTGCACCCCCACCACCAGCGGCGAGGACGTGCGGGAC
TTCGCCAAGGTGCTGAAGAACAAGTTCCGGACCAAGCGGTACTTCGCCAAGCACCCCCGGATGGGCTACCTGCCC
GTGCAGACCGTGCTGGAGGGCGACAACATGGAGACCCCCGTGACCCTGATCAACTTCTGGCCCGTGGACAGCGCC
CCCGCCAGCAGCCCCCAGCTGAGCCACGACGACACCCACAGCCGGATCGAGCACTACGCCAGCCGGCTGGCCGAG
ATGGAGAACAGCAACGGCAGCTACCTGAACGACAGCATCAGCCCCAACGAGAGCATCGACGACGAGCACCTGCTG
ATCCAGCACTACTGCCAGAGCCTGAACCAGGACAGCCCCCTGAGCCAGCCCCGGAGC
Utrophin N-terminus through R2_Dystrophin R23 to end of 1st Coiled Coil:
Human sequences without codon alterations:
(SEQ ID NO: 114)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCGCCGCTAGCGCCAGTTCTGACCAGTGGAAGCGTCTGCACCTTTCTCTGCAGGAACTTCTGGTGTGGCTA
CAGCTGAAAGATGATGAATTAAGCCGGCAGGCACCTATTGGAGGCGACTTTCCAGCAGTTCAGAAGCAGAACGAT
GTACATAGGGCCTTCAAGAGGGAATTGAAAACTAAAGAACCTGTAATCATGAGTACTCTTGAGACTGTACGAATA
TTTCTGACAGAGCAGCCTTTGGAAGGACTAGAGAAACTCTACCAGGAGCCCAGAGAGCTGCCTCCTGAGGAGAGA
GCCCAGAATGTCACTCGGCTTCTACGAAAGCAGGCTGAGGAGGTCAATACTGAGTGGGAAAAATTGAACCTGCAC
TCCGCTGACTGGCAGAGAAAAATAGATGAGACCCTTGAAAGACTCCGGGAACTTCAAGAGGCCACGGATGAGCTG
GACCTCAAGCTGCGCCAAGCTGAGGTGATCAAGGGATCCTGGCAGCCCGTGGGCGATCTCCTCATTGACTCTCTC
CAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAAC
ACCAGATGGAAGCTTCTGCAGGTGGCCGTCGAGGACCGAGTCAGGCAGCTGCATGAAGCCCACAGGGACTTTGGT
CCAGCATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTTTAGCT
GACCTGAATAATGTCAGATTCTCAGCTTATAGGACTGCCATGAAACTCCGAAGACTGCAGAAGGCCCTTTGCTTG
GATCTCTTGAGCCTGTCAGCTGCATGTGATGCCTTGGACCAGCACAACCTCAAGCAAAATGACCAGCCCATGGAT
ATCCTGCAGATTATTAATTGTTTGACCACTATTTATGACCGCCTGGAGCAAGAGCACAACAATTTGGTCAACGTC
CCTCTCTGCGTGGATATGTGTCTGAACTGGCTGCTGAATGTTTATGATACGGGACGAACAGGGAGGATCCGTGTC
CTGTCTTTTAAAACTGGCATCATTTCCCTGTGTAAAGCACATTTGGAAGACAAGTACAGATACCTTTTCAAGCAA
GTGGCAAGTTCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCC
GTCCTGCACAGAGTGGCTGCTGCAGAAACTGCCAAGCATCAGGCCAAATGTAACATCTGCAAAGAGTGTCCAATC
ATTGGATTCAGGTACAGGAGTCTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTACATCAGGAGAAGATGTTCGAGAC
TTTGCCAAGGTACTAAAAAACAAATTTCGAACCAAAAGGTATTTTGCGAAGCATCCCCGAATGGGCTACCTGCCA
GTGCAGACTGTCTTAGAGGGGGACAACATGGAAACTCCCGTTACTCTGATCAACTTCTGGCCAGTAGATTCTGCG
CCTGCCTCGTCCCCTCAGCTTTCACACGATGATACTCATTCACGCATTGAACATTATGCTAGCAGGCTAGCAGAA
ATGGAAAACAGCAATGGATCTTATCTAAATGATAGCATCTCTCCTAATGAGAGCATAGATGATGAACATTTGTTA
ATCCAGCATTACTGCCAAAGTTTGAACCAGGACTCCCCCCTGAGCCAGCCTCGTAGTCCTGCCCAGATCTTGATT
TCCTTAGAGAGTGAGGAAAGAGGGGAGCTAGAGAGAATCCTAGCAGATCTTGAGGAAGAAAACAGGAATCTGCAA
GCAGAATATGACCGTCTAAAGCAGCAGCACGAACATAAAGGCCTGTCCCCACTGCCGTCCCCTCCTGAAATGATG
CCCACCTCTCCCCAGAGTCCC
Human sequences with codon optimization:
(SEQ ID NO: 115)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCGCCGCTAGCGCCAGCAGCGACCAGTGGAAGCGGCTGCACCTGAGCCTGCAGGAGCTGCTGGTGTGGCTG
CAGCTGAAGGACGACGAGCTGAGCCGGCAGGCCCCCATCGGCGGCGACTTCCCCGCCGTGCAGAAGCAGAACGAC
GTGCACCGGGCCTTCAAGCGGGAGCTGAAGACCAAGGAGCCCGTGATCATGAGCACCCTGGAGACCGTGCGGATC
TTCCTGACCGAGCAGCCCCTGGAGGGCCTGGAGAAGCTGTACCAGGAGCCCCGGGAGCTGCCCCCCGAGGAGCGG
GCCCAGAACGTGACCCGGCTGCTGCGGAAGCAGGCCGAGGAGGTGAACACCGAGTGGGAGAAGCTGAACCTGCAC
AGCGCCGACTGGCAGCGGAAGATCGACGAGACCCTGGAGCGGCTGCAGGAGCTGCAGGAGGCCACCGACGAGCTG
GACCTGAAGCTGCGGCAGGCCGAGGTGATCAAGGGCAGCTGGCAGCCCGTGGGCGACCTGCTGATCGACAGCCTG
CAGGACCACCTGGAGAAGGTGAAGGCCCTGCGGGGCGAGATCGCCCCCCTGAAGGAGAACGTGAGCCACGTGAAC
GACCTGGCCCGGCAGCTGACCACCCTGGGCATCCAGCTGAGCCCCTACAACCTGAGCACCCTGGAGGACCTGAAC
ACCCGGTGGAAGCTGCTGCAGGTGGCCGTGGAGGACCGGGTGCGGCAGCTGCACGAGGCCCACCGGGACTTCGGC
CCCGCCAGCCAGCACTTCCTGAGCACCAGCGTGCAGGGCCCCTGGGAGCGGGCCATCAGCCCCAACAAGGTGCCC
TACTACATCAACCACGAGACCCAGACCACCTGCTGGGACCACCCCAAGATGACCGAGCTGTACCAGAGCCTGGCC
GACCTGAACAACGTGCGGTTCAGCGCCTACCGGACCGCCATGAAGCTGCGGCGGCTGCAGAAGGCCCTGTGCCTG
GACCTGCTGAGCCTGAGCGCCGCCTGCGACGCCCTGGACCAGCACAACCTGAAGCAGAACGACCAGCCCATGGAC
ATCCTGCAGATCATCAACTGCCTGACCACCATCTACGACCGGCTGGAGCAGGAGCACAACAACCTGGTGAACGTG
CCCCTGTGCGTGGACATGTGCCTGAACTGGCTGCTGAACGTGTACGACACCGGCCGGACCGGCCGGATCCGGGTG
CTGAGCTTCAAGACCGGCATCATCAGCCTGTGCAAGGCCCACCTGGAGGACAAGTACCGGTACCTGTTCAAGCAG
GTGGCCAGCAGCACCGGCTTCTGCGACCAGCGGCGGCTGGGCCTGCTGCTGCACGACAGCATCCAGATCCCCCGG
CAGCTGGGCGAGGTGGCCAGCTTCGGCGGCAGCAACATCGAGCCCAGCGTGCGGAGCTGCTTCCAGTTCGCCAAC
AACAAGCCCGAGATCGAGGCCGCCCTGTTCCTGGACTGGATGCGGCTGGAGCCCCAGAGCATGGTGTGGCTGCCC
GTGCTGCACCGGGTGGCCGCCGCCGAGACCGCCAAGCACCAGGCCAAGTGCAACATCTGCAAGGAGTGCCCCATC
ATCGGCTTCCGGTACCGGAGCCTGAAGCACTTCAACTACGACATCTGCCAGAGCTGCTTCTTCAGCGGCCGGGTG
GCCAAGGGCCACAAGATGCACTACCCCATGGTGGAGTACTGCACCCCCACCACCAGCGGCGAGGACGTGCGGGAC
TTCGCCAAGGTGCTGAAGAACAAGTTCCGGACCAAGCGGTACTTCGCCAAGCACCCCCGGATGGGCTACCTGCCC
GTGCAGACCGTGCTGGAGGGCGACAACATGGAGACCCCCGTGACCCTGATCAACTTCTGGCCCGTGGACAGCGCC
CCCGCCAGCAGCCCCCAGCTGAGCCACGACGACACCCACAGCCGGATCGAGCACTACGCCAGCCGGCTGGCCGAG
ATGGAGAACAGCAACGGCAGCTACCTGAACGACAGCATCAGCCCCAACGAGAGCATCGACGACGAGCACCTGCTG
ATCCAGCACTACTGCCAGAGCCTGAACCAGGACAGCCCCCTGAGCCAGCCCCGGAGCCCCGCCCAGATCCTGATC
AGCCTGGAGAGCGAGGAGCGGGGCGAGCTGGAGCGGATCCTGGCCGACCTGGAGGAGGAGAACCGGAACCTGCAG
GCCGAGTACGACCGGCTGAAGCAGCAGCACGAGCACAAGGGCCTGAGCCCCCTGCCCAGCCCCCCCGAGATGATG
CCCACCAGCCCCCAGAGCCCC
Utrophin N-terminus through R3_Dystrophin R23 + R24 + H4 to end of syntrophin
region:
Human sequences without codon alterations:
(SEQ ID NO: 116)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCTGCCGCTAGCGCCAGTTCTGACCAGTGGAAGCGTCTG
CACCTTTCTCTGCAGGAACTTCTGGTGTGGCTACAGCTGAAAGATGATGAATTAAGCCGGCAGGCACCTATTGGA
GGCGACTTTCCAGCAGTTCAGAAGCAGAACGATGTACATAGGGCCTTCAAGAGGGAATTGAAAACTAAAGAACCT
GTAATCATGAGTACTCTTGAGACTGTACGAATATTTCTGACAGAGCAGCCTTTGGAAGGACTAGAGAAACTCTAC
CAGGAGCCCAGAGAGCTGCCTCCTGAGGAGAGAGCCCAGAATGTCACTCGGCTTCTACGAAAGCAGGCTGAGGAG
GTCAATACTGAGTGGGAAAAATTGAACCTGCACTCCGCTGACTGGCAGAGAAAAATAGATGAGACCCTTGAAAGA
CTCCGGGAACTTCAAGAGGCCACGGATGAGCTGGACCTCAAGCTGCGCCAAGCTGAGGTGATCAAGGGATCCTGG
CAGCCCGTGGGCGATCTCCTCATTGACTCTCTCCAAGATCACCTCGAGAAAGTCAAGGCACTTCGAGGAGAAATT
GCGCCTCTGAAAGAGAACGTGAGCCACGTCAATGACCTTGCTCGCCAGCTTACCACTTTGGGCATTCAGCTCTCA
CCGTATAACCTCAGCACTCTGGAAGACCTGAACACCAGATGGAAGCTTCTGCAGGTGGCCGTCGAGGACCGAGTC
AGGCAGCTGCATGAAGCCCACAGGGACTTTGGTCCAGCATCTCAGCACTTTCTTTCCACGTCTGTCCAGGGTCCC
TGGGAGAGAGCCATCTCGCCAAACAAAGTGCCCTACTATATCAACCACGAGACTCAAACAACTTGCTGGGACCAT
CCCAAAATGACAGAGCTCTACCAGTCTTTAGCTGACCTGAATAATGTCAGATTCTCAGCTTATAGGACTGCCATG
AAACTCCGAAGACTGCAGAAGGCCCTTTGCTTGGATCTCTTGAGCCTGTCAGCTGCATGTGATGCCTTGGACCAG
CACAACCTCAAGCAAAATGACCAGCCCATGGATATCCTGCAGATTATTAATTGTTTGACCACTATTTATGACCGC
CTGGAGCAAGAGCACAACAATTTGGTCAACGTCCCTCTCTGCGTGGATATGTGTCTGAACTGGCTGCTGAATGTT
TATGATACGGGACGAACAGGGAGGATCCGTGTCCTGTCTTTTAAAACTGGCATCATTTCCCTGTGTAAAGCACAT
TTGGAAGACAAGTACAGATACCTTTTCAAGCAAGTGGCAAGTTCAACAGGATTTTGTGACCAGCGCAGGCTGGGC
CTCCTTCTGCATGATTCTATCCAAATTCCAAGACAGTTGGGTGAAGTTGCATCCTTTGGGGGCAGTAACATTGAG
CCAAGTGTCCGGAGCTGCTTCCAATTTGCTAATAATAAGCCAGAGATCGAAGCGGCCCTCTTCCTAGACTGGATG
AGACTGGAACCCCAGTCCATGGTGTGGCTGCCCGTCCTGCACAGAGTGGCTGCTGCAGAAACTGCCAAGCATCAG
GCCAAATGTAACATCTGCAAAGAGTGTCCAATCATTGGATTCAGGTACAGGAGTCTAAAGCACTTTAATTATGAC
ATCTGCCAAAGCTGCTTTTTTTCTGGTCGAGTTGCAAAAGGCCATAAAATGCACTATCCCATGGTGGAATATTGC
ACTCCGACTACATCAGGAGAAGATGTTCGAGACTTTGCCAAGGTACTAAAAAACAAATTTCGAACCAAAAGGTAT
TTTGCGAAGCATCCCCGAATGGGCTACCTGCCAGTGCAGACTGTCTTAGAGGGGGACAACATGGAAACTCCCGTT
ACTCTGATCAACTTCTGGCCAGTAGATTCTGCGCCTGCCTCGTCCCCTCAGCTTTCACACGATGATACTCATTCA
CGCATTGAACATTATGCTAGCAGGCTAGCAGAAATGGAAAACAGCAATGGATCTTATCTAAATGATAGCATCTCT
CCTAATGAGAGCATAGATGATGAACATTTGTTAATCCAGCATTACTGCCAAAGTTTGAACCAGGACTCCCCCCTG
AGCCAGCCTCGTAGT
Human sequences with codon optimization:
(SEQ ID NO: 117)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGCCGCTAGCGCCAGCAGCGACCAGTGGAAGCGGCTG
CACCTGAGCCTGCAGGAGCTGCTGGTGTGGCTGCAGCTGAAGGACGACGAGCTGAGCCGGCAGGCCCCCATCGGC
GGCGACTTCCCCGCCGTGCAGAAGCAGAACGACGTGCACCGGGCCTTCAAGCGGGAGCTGAAGACCAAGGAGCCC
GTGATCATGAGCACCCTGGAGACCGTGCGGATCTTCCTGACCGAGCAGCCCCTGGAGGGCCTGGAGAAGCTGTAC
CAGGAGCCCCGGGAGCTGCCCCCCGAGGAGCGGGCCCAGAACGTGACCCGGCTGCTGCGGAAGCAGGCCGAGGAG
GTGAACACCGAGTGGGAGAAGCTGAACCTGCACAGCGCCGACTGGCAGCGGAAGATCGACGAGACCCTGGAGCGG
CTGCAGGAGCTGCAGGAGGCCACCGACGAGCTGGACCTGAAGCTGCGGCAGGCCGAGGTGATCAAGGGCAGCTGG
CAGCCCGTGGGCGACCTGCTGATCGACAGCCTGCAGGACCACCTGGAGAAGGTGAAGGCCCTGCGGGGCGAGATC
GCCCCCCTGAAGGAGAACGTGAGCCACGTGAACGACCTGGCCCGGCAGCTGACCACCCTGGGCATCCAGCTGAGC
CCCTACAACCTGAGCACCCTGGAGGACCTGAACACCCGGTGGAAGCTGCTGCAGGTGGCCGTGGAGGACCGGGTG
CGGCAGCTGCACGAGGCCCACCGGGACTTCGGCCCCGCCAGCCAGCACTTCCTGAGCACCAGCGTGCAGGGCCCC
TGGGAGCGGGCCATCAGCCCCAACAAGGTGCCCTACTACATCAACCACGAGACCCAGACCACCTGCTGGGACCAC
CCCAAGATGACCGAGCTGTACCAGAGCCTGGCCGACCTGAACAACGTGCGGTTCAGCGCCTACCGGACCGCCATG
AAGCTGCGGCGGCTGCAGAAGGCCCTGTGCCTGGACCTGCTGAGCCTGAGCGCCGCCTGCGACGCCCTGGACCAG
CACAACCTGAAGCAGAACGACCAGCCCATGGACATCCTGCAGATCATCAACTGCCTGACCACCATCTACGACCGG
CTGGAGCAGGAGCACAACAACCTGGTGAACGTGCCCCTGTGCGTGGACATGTGCCTGAACTGGCTGCTGAACGTG
TACGACACCGGCCGGACCGGCCGGATCCGGGTGCTGAGCTTCAAGACCGGCATCATCAGCCTGTGCAAGGCCCAC
CTGGAGGACAAGTACCGGTACCTGTTCAAGCAGGTGGCCAGCAGCACCGGCTTCTGCGACCAGCGGCGGCTGGGC
CTGCTGCTGCACGACAGCATCCAGATCCCCCGGCAGCTGGGCGAGGTGGCCAGCTTCGGCGGCAGCAACATCGAG
CCCAGCGTGCGGAGCTGCTTCCAGTTCGCCAACAACAAGCCCGAGATCGAGGCCGCCCTGTTCCTGGACTGGATG
CGGCTGGAGCCCCAGAGCATGGTGTGGCTGCCCGTGCTGCACCGGGTGGCCGCCGCCGAGACCGCCAAGCACCAG
GCCAAGTGCAACATCTGCAAGGAGTGCCCCATCATCGGCTTCCGGTACCGGAGCCTGAAGCACTTCAACTACGAC
ATCTGCCAGAGCTGCTTCTTCAGCGGCCGGGTGGCCAAGGGCCACAAGATGCACTACCCCATGGTGGAGTACTGC
ACCCCCACCACCAGCGGCGAGGACGTGCGGGACTTCGCCAAGGTGCTGAAGAACAAGTTCCGGACCAAGCGGTAC
TTCGCCAAGCACCCCCGGATGGGCTACCTGCCCGTGCAGACCGTGCTGGAGGGCGACAACATGGAGACCCCCGTG
ACCCTGATCAACTTCTGGCCCGTGGACAGCGCCCCCGCCAGCAGCCCCCAGCTGAGCCACGACGACACCCACAGC
CGGATCGAGCACTACGCCAGCCGGCTGGCCGAGATGGAGAACAGCAACGGCAGCTACCTGAACGACAGCATCAGC
CCCAACGAGAGCATCGACGACGAGCACCTGCTGATCCAGCACTACTGCCAGAGCCTGAACCAGGACAGCCCCCTG
AGCCAGCCCCGGAGC
Utrophin N-terminus through R2_Dystrophin R23 to end:
Human sequences without codon alterations:
(SEQ ID NO: 118)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCGCCGCTAGCGCCAGTTCTGACCAGTGGAAGCGTCTGCACCTTTCTCTGCAGGAACTTCTGGTGTGGCTA
CAGCTGAAAGATGATGAATTAAGCCGGCAGGCACCTATTGGAGGCGACTTTCCAGCAGTTCAGAAGCAGAACGAT
GTACATAGGGCCTTCAAGAGGGAATTGAAAACTAAAGAACCTGTAATCATGAGTACTCTTGAGACTGTACGAATA
TTTCTGACAGAGCAGCCTTTGGAAGGACTAGAGAAACTCTACCAGGAGCCCAGAGAGCTGCCTCCTGAGGAGAGA
GCCCAGAATGTCACTCGGCTTCTACGAAAGCAGGCTGAGGAGGTCAATACTGAGTGGGAAAAATTGAACCTGCAC
TCCGCTGACTGGCAGAGAAAAATAGATGAGACCCTTGAAAGACTCCGGGAACTTCAAGAGGCCACGGATGAGCTG
GACCTCAAGCTGCGCCAAGCTGAGGTGATCAAGGGATCCTGGCAGCCCGTGGGCGATCTCCTCATTGACTCTCTC
CAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAAC
ACCAGATGGAAGCTTCTGCAGGTGGCCGTCGAGGACCGAGTCAGGCAGCTGCATGAAGCCCACAGGGACTTTGGT
CCAGCATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTTTAGCT
GACCTGAATAATGTCAGATTCTCAGCTTATAGGACTGCCATGAAACTCCGAAGACTGCAGAAGGCCCTTTGCTTG
GATCTCTTGAGCCTGTCAGCTGCATGTGATGCCTTGGACCAGCACAACCTCAAGCAAAATGACCAGCCCATGGAT
ATCCTGCAGATTATTAATTGTTTGACCACTATTTATGACCGCCTGGAGCAAGAGCACAACAATTTGGTCAACGTC
CCTCTCTGCGTGGATATGTGTCTGAACTGGCTGCTGAATGTTTATGATACGGGACGAACAGGGAGGATCCGTGTC
CTGTCTTTTAAAACTGGCATCATTTCCCTGTGTAAAGCACATTTGGAAGACAAGTACAGATACCTTTTCAAGCAA
GTGGCAAGTTCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCC
GTCCTGCACAGAGTGGCTGCTGCAGAAACTGCCAAGCATCAGGCCAAATGTAACATCTGCAAAGAGTGTCCAATC
ATTGGATTCAGGTACAGGAGTCTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTACATCAGGAGAAGATGTTCGAGAC
TTTGCCAAGGTACTAAAAAACAAATTTCGAACCAAAAGGTATTTTGCGAAGCATCCCCGAATGGGCTACCTGCCA
GTGCAGACTGTCTTAGAGGGGGACAACATGGAAACTCCCGTTACTCTGATCAACTTCTGGCCAGTAGATTCTGCG
CCTGCCTCGTCCCCTCAGCTTTCACACGATGATACTCATTCACGCATTGAACATTATGCTAGCAGGCTAGCAGAA
ATGGAAAACAGCAATGGATCTTATCTAAATGATAGCATCTCTCCTAATGAGAGCATAGATGATGAACATTTGTTA
ATCCAGCATTACTGCCAAAGTTTGAACCAGGACTCCCCCCTGAGCCAGCCTCGTAGTCCTGCCCAGATCTTGATT
TCCTTAGAGAGTGAGGAAAGAGGGGAGCTAGAGAGAATCCTAGCAGATCTTGAGGAAGAAAACAGGAATCTGCAA
GCAGAATATGACCGTCTAAAGCAGCAGCACGAACATAAAGGCCTGTCCCCACTGCCGTCCCCTCCTGAAATGATG
CCCACCTCTCCCCAGAGTCCCCGGGATGCTGAGCTCATTGCTGAGGCCAAGCTACTGCGTCAACACAAAGGCCGC
CTGGAAGCCAGGATGCAAATCCTGGAAGACCACAATAAACAGCTGGAGTCACAGTTACACAGGCTAAGGCAGCTG
CTGGAGCAACCCCAGGCAGAGGCCAAAGTGAATGGCACAACGGTGTCCTCTCCTTCTACCTCTCTACAGAGGTCC
GACAGCAGTCAGCCTATGCTGCTCCGAGTGGTTGGCAGTCAAACTTCGGACTCCATGGGTGAGGAAGATCTTCTC
AGTCCTCCCCAGGACACAAGCACAGGGTTAGAGGAGGTGATGGAGCAACTCAACAACTCCTTCCCTAGTTCAAGA
GGAAGAAATACCCCTGGAAAGCCAATGAGAGAGGACACAATG
Human sequences with codon optimization:
(SEQ ID NO: 119)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCGCCGCTAGCGCCAGCAGCGACCAGTGGAAGCGGCTGCACCTGAGCCTGCAGGAGCTGCTGGTGTGGCTG
CAGCTGAAGGACGACGAGCTGAGCCGGCAGGCCCCCATCGGCGGCGACTTCCCCGCCGTGCAGAAGCAGAACGAC
GTGCACCGGGCCTTCAAGCGGGAGCTGAAGACCAAGGAGCCCGTGATCATGAGCACCCTGGAGACCGTGCGGATC
TTCCTGACCGAGCAGCCCCTGGAGGGCCTGGAGAAGCTGTACCAGGAGCCCCGGGAGCTGCCCCCCGAGGAGCGG
GCCCAGAACGTGACCCGGCTGCTGCGGAAGCAGGCCGAGGAGGTGAACACCGAGTGGGAGAAGCTGAACCTGCAC
AGCGCCGACTGGCAGAGAAAAATAGATGAGACCCTTGAAAGACTCCGGGAACTTCAAGAGGCCACGGATGAGCTG
GACCTCAAGCTGCGCCAAGCTGAGGTGATCAAGGGATCCTGGCAGCCCGTGGGCGATCTCCTCATTGACTCTCTC
CAAGATCACCTCGAGAAAGTCAAGGCACTTCGAGGAGAAATTGCGCCTCTGAAAGAGAACGTGAGCCACGTCAAT
GACCTTGCTCGCCAGCTTACCACTTTGGGCATTCAGCTCTCACCGTATAACCTCAGCACTCTGGAAGACCTGAAC
ACCAGATGGAAGCTTCTGCAGGTGGCCGTCGAGGACCGAGTCAGGCAGCTGCATGAAGCCCACAGGGACTTTGGT
CCAGCATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTTTAGCT
GACCTGAATAATGTCAGATTCTCAGCTTATAGGACTGCCATGAAACTCCGAAGACTGCAGAAGGCCCTTTGCTTG
GATCTCTTGAGCCTGTCAGCTGCATGTGATGCCTTGGACCAGCACAACCTCAAGCAAAATGACCAGCCCATGGAT
ATCCTGCAGATTATTAATTGTTTGACCACTATTTATGACCGCCTGGAGCAAGAGCACAACAATTTGGTCAACGTC
CCTCTCTGCGTGGATATGTGTCTGAACTGGCTGCTGAATGTTTATGATACGGGACGAACAGGGAGGATCCGTGTC
CTGTCTTTTAAAACTGGCATCATTTCCCTGTGTAAAGCACATTTGGAAGACAAGTACAGATACCTTTTCAAGCAA
GTGGCAAGTTCAACAGGATTTTGTGACCAGCGCAGGCTGGGCCTCCTTCTGCATGATTCTATCCAAATTCCAAGA
CAGTTGGGTGAAGTTGCATCCTTTGGGGGCAGTAACATTGAGCCAAGTGTCCGGAGCTGCTTCCAATTTGCTAAT
AATAAGCCAGAGATCGAAGCGGCCCTCTTCCTAGACTGGATGAGACTGGAACCCCAGTCCATGGTGTGGCTGCCC
GTCCTGCACAGAGTGGCTGCTGCAGAAACTGCCAAGCATCAGGCCAAATGTAACATCTGCAAAGAGTGTCCAATC
ATTGGATTCAGGTACAGGAGTCTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTACATCAGGAGAAGATGTTCGAGAC
TTTGCCAAGGTACTAAAAAACAAATTTCGAACCAAAAGGTATTTTGCGAAGCATCCCCGAATGGGCTACCTGCCA
GTGCAGACTGTCTTAGAGGGGGACAACATGGAAACTCCCGTTACTCTGATCAACTTCTGGCCAGTAGATTCTGCG
CCTGCCTCGTCCCCTCAGCTTTCACACGATGATACTCATTCACGCATTGAACATTATGCTAGCAGGCTAGCAGAA
ATGGAAAACAGCAATGGATCTTATCTAAATGATAGCATCTCTCCTAATGAGAGCATAGATGATGAACATTTGTTA
ATCCAGCATTACTGCCAAAGTTTGAACCAGGACTCCCCCCTGAGCCAGCCTCGTAGTCCTGCCCAGATCTTGATT
TCCTTAGAGAGTGAGGAAAGAGGGGAGCTAGAGAGAATCCTAGCAGATCTTGAGGAAGAAAACAGGAATCTGCAA
GCAGAATATGACCGTCTAAAGCAGCAGCACGAACATAAAGGCCTGTCCCCACTGCCGTCCCCTCCTGAAATGATG
CCCACCTCTCCCCAGAGTCCCCGGGATGCTGAGCTCATTGCTGAGGCCAAGCTACTGCGTCAACACAAAGGCCGC
CTGGAAGCCAGGATGCAAATCCTGGAAGACCACAATAAACAGCTGGAGTCACAGTTACACAGGCTAAGGCAGCTG
CTGGAGCAACCCCAGGCAGAGGCCAAAGTGAATGGCACAACGGTGTCCTCTCCTTCTACCTCTCTACAGAGGTCC
GACAGCAGTCAGCCTATGCTGCTCCGAGTGGTTGGCAGTCAAACTTCGGACTCCATGGGTGAGGAAGATCTTCTC
AGTCCTCCCCAGGACACAAGCACAGGGTTAGAGGAGGTGATGGAGCAACTCAACAACTCCTTCCCTAGTTCAAGA
GGAAGAAATACCCCTGGAAAGCCAATGAGAGAGGACACAATG
Utrophin N-terminus through R3_Dystrophin R24 to end:
Human sequences without codon alterations:
(SEQ ID NO: 135)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCTCAGAGAAAAATAGATGAGACCCTTGAAAGACTCCGG
GAACTTCAAGAGGCCACGGATGAGCTGGACCTCAAGCTGCGCCAAGCTGAGGTGATCAAGGGATCCTGGCAGCCC
GTGGGCGATCTCCTCATTGACTCTCTCCAAGATCACCTCGAGAAAGTCAAGGCACTTCGAGGAGAAATTGCGCCT
CTGAAAGAGAACGTGAGCCACGTCAATGACCTTGCTCGCCAGCTTACCACTTTGGGCATTCAGCTCTCACCGTAT
AACCTCAGCACTCTGGAAGACCTGAACACCAGATGGAAGCTTCTGCAGGTGGCCGTCGAGGACCGAGTCAGGCAG
CTGCATGAAGCCCACAGGGACTTTGGTCCAGCATCTCAGCACTTTCTTTCCACGTCTGTCCAGGGTCCCTGGGAG
AGAGCCATCTCGCCAAACAAAGTGCCCTACTATATCAACCACGAGACTCAAACAACTTGCTGGGACCATCCCAAA
ATGACAGAGCTCTACCAGTCTTTAGCTGACCTGAATAATGTCAGATTCTCAGCTTATAGGACTGCCATGAAACTC
CGAAGACTGCAGAAGGCCCTTTGCTTGGATCTCTTGAGCCTGTCAGCTGCATGTGATGCCTTGGACCAGCACAAC
CTCAAGCAAAATGACCAGCCCATGGATATCCTGCAGATTATTAATTGTTTGACCACTATTTATGACCGCCTGGAG
CAAGAGCACAACAATTTGGTCAACGTCCCTCTCTGCGTGGATATGTGTCTGAACTGGCTGCTGAATGTTTATGAT
ACGGGACGAACAGGGAGGATCCGTGTCCTGTCTTTTAAAACTGGCATCATTTCCCTGTGTAAAGCACATTTGGAA
GACAAGTACAGATACCTTTTCAAGCAAGTGGCAAGTTCAACAGGATTTTGTGACCAGCGCAGGCTGGGCCTCCTT
CTGCATGATTCTATCCAAATTCCAAGACAGTTGGGTGAAGTTGCATCCTTTGGGGGCAGTAACATTGAGCCAAGT
GTCCGGAGCTGCTTCCAATTTGCTAATAATAAGCCAGAGATCGAAGCGGCCCTCTTCCTAGACTGGATGAGACTG
GAACCCCAGTCCATGGTGTGGCTGCCCGTCCTGCACAGAGTGGCTGCTGCAGAAACTGCCAAGCATCAGGCCAAA
TGTAACATCTGCAAAGAGTGTCCAATCATTGGATTCAGGTACAGGAGTCTAAAGCACTTTAATTATGACATCTGC
CAAAGCTGCTTTTTTTCTGGTCGAGTTGCAAAAGGCCATAAAATGCACTATCCCATGGTGGAATATTGCACTCCG
ACTACATCAGGAGAAGATGTTCGAGACTTTGCCAAGGTACTAAAAAACAAATTTCGAACCAAAAGGTATTTTGCG
AAGCATCCCCGAATGGGCTACCTGCCAGTGCAGACTGTCTTAGAGGGGGACAACATGGAAACTCCCGTTACTCTG
ATCAACTTCTGGCCAGTAGATTCTGCGCCTGCCTCGTCCCCTCAGCTTTCACACGATGATACTCATTCACGCATT
GAACATTATGCTAGCAGGCTAGCAGAAATGGAAAACAGCAATGGATCTTATCTAAATGATAGCATCTCTCCTAAT
GAGAGCATAGATGATGAACATTTGTTAATCCAGCATTACTGCCAAAGTTTGAACCAGGACTCCCCCCTGAGCCAG
CCTCGTAGTCCTGCCCAGATCTTGATTTCCTTAGAGAGTGAGGAAAGAGGGGAGCTAGAGAGAATCCTAGCAGAT
CTTGAGGAAGAAAACAGGAATCTGCAAGCAGAATATGACCGTCTAAAGCAGCAGCACGAACATAAAGGCCTGTCC
CCACTGCCGTCCCCTCCTGAAATGATGCCCACCTCTCCCCAGAGTCCCCGGGATGCTGAGCTCATTGCTGAGGCC
AAGCTACTGCGTCAACACAAAGGCCGCCTGGAAGCCAGGATGCAAATCCTGGAAGACCACAATAAACAGCTGGAG
TCACAGTTACACAGGCTAAGGCAGCTGCTGGAGCAACCCCAGGCAGAGGCCAAAGTGAATGGCACAACGGTGTCC
TCTCCTTCTACCTCTCTACAGAGGTCCGACAGCAGTCAGCCTATGCTGCTCCGAGTGGTTGGCAGTCAAACTTCG
GACTCCATGGGTGAGGAAGATCTTCTCAGTCCTCCCCAGGACACAAGCACAGGGTTAGAGGAGGTGATGGAGCAA
CTCAACAACTCCTTCCCTAGTTCAAGAGGAAGAAATACCCCTGGAAAGCCAATGAGAGAGGACACAATG
Human sequences with codon optimization:
(SEQ ID NO: 136)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGCCGCTAGCGCCCAGAGAAAAATAGATGAGACCCTT
GAAAGACTCCGGGAACTTCAAGAGGCCACGGATGAGCTGGACCTCAAGCTGCGCCAAGCTGAGGTGATCAAGGGA
TCCTGGCAGCCCGTGGGCGATCTCCTCATTGACTCTCTCCAAGATCACCTCGAGAAAGTCAAGGCACTTCGAGGA
GAAATTGCGCCTCTGAAAGAGAACGTGAGCCACGTCAATGACCTTGCTCGCCAGCTTACCACTTTGGGCATTCAG
CTCTCACCGTATAACCTCAGCACTCTGGAAGACCTGAACACCAGATGGAAGCTTCTGCAGGTGGCCGTCGAGGAC
CGAGTCAGGCAGCTGCATGAAGCCCACAGGGACTTTGGTCCAGCATCTCAGCACTTTCTTTCCACGTCTGTCCAG
GGTCCCTGGGAGAGAGCCATCTCGCCAAACAAAGTGCCCTACTATATCAACCACGAGACTCAAACAACTTGCTGG
GACCATCCCAAAATGACAGAGCTCTACCAGTCTTTAGCTGACCTGAATAATGTCAGATTCTCAGCTTATAGGACT
GCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTAT
GACCGCCTGGAGCAAGAGCACAACAATTTGGTCAACGTCCCTCTCTGCGTGGATATGTGTCTGAACTGGCTGCTG
AATGTTTATGATACGGGACGAACAGGGAGGATCCGTGTCCTGTCTTTTAAAACTGGCATCATTTCCCTGTGTAAA
GCACATTTGGAAGACAAGTACAGATACCTTTTCAAGCAAGTGGCAAGTTCAACAGGATTTTGTGACCAGCGCAGG
CTGGGCCTCCTTCTGCATGATTCTATCCAAATTCCAAGACAGTTGGGTGAAGTTGCATCCTTTGGGGGCAGTAAC
ATTGAGCCAAGTGTCCGGAGCTGCTTCCAATTTGCTAATAATAAGCCAGAGATCGAAGCGGCCCTCTTCCTAGAC
TGGATGAGACTGGAACCCCAGTCCATGGTGTGGCTGCCCGTCCTGCACAGAGTGGCTGCTGCAGAAACTGCCAAG
CATCAGGCCAAATGTAACATCTGCAAAGAGTGTCCAATCATTGGATTCAGGTACAGGAGTCTAAAGCACTTTAAT
TATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAA
AGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACT
CATTCACGCATTGAACATTATGCTAGCAGGCTAGCAGAAATGGAAAACAGCAATGGATCTTATCTAAATGATAGC
ATCTCTCCTAATGAGAGCATAGATGATGAACATTTGTTAATCCAGCATTACTGCCAAAGTTTGAACCAGGACTCC
CCCCTGAGCCAGCCTCGTAGTCCTGCCCAGATCTTGATTTCCTTAGAGAGTGAGGAAAGAGGGGAGCTAGAGAGA
ATCCTAGCAGATCTTGAGGAAGAAAACAGGAATCTGCAAGCAGAATATGACCGTCTAAAGCAGCAGCACGAACAT
AAAGGCCTGTCCCCACTGCCGTCCCCTCCTGAAATGATGCCCACCTCTCCCCAGAGTCCCCGGGATGCTGAGCTC
ATTGCTGAGGCCAAGCTACTGCGTCAACACAAAGGCCGCCTGGAAGCCAGGATGCAAATCCTGGAAGACCACAAT
AAACAGCTGGAGTCACAGTTACACAGGCTAAGGCAGCTGCTGGAGCAACCCCAGGCAGAGGCCAAAGTGAATGGC
ACAACGGTGTCCTCTCCTTCTACCTCTCTACAGAGGTCCGACAGCAGTCAGCCTATGCTGCTCCGAGTGGTTGGC
AGTCAAACTTCGGACTCCATGGGTGAGGAAGATCTTCTCAGTCCTCCCCAGGACACAAGCACAGGGTTAGAGGAG
GTGATGGAGCAACTCAACAACTCCTTCCCTAGTTCAAGAGGAAGAAATACCCCTGGAAAGCCAATGAGAGAGGAC
ACAATG
Utrophin N-terminus through R3_Dystrophin R24 + H4 to end of syntrophin region:
Human sequences without codon alterations:
(SEQ ID NO: 137)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCTGCCGCTAGCGCCCAGAGAAAAATAGATGAGACCCTT
GAAAGACTCCGGGAACTTCAAGAGGCCACGGATGAGCTGGACCTCAAGCTGCGCCAAGCTGAGGTGATCAAGGGA
TCCTGGCAGCCCGTGGGCGATCTCCTCATTGACTCTCTCCAAGATCACCTCGAGAAAGTCAAGGCACTTCGAGGA
GAAATTGCGCCTCTGAAAGAGAACGTGAGCCACGTCAATGACCTTGCTCGCCAGCTTACCACTTTGGGCATTCAG
CTCTCACCGTATAACCTCAGCACTCTGGAAGACCTGAACACCAGATGGAAGCTTCTGCAGGTGGCCGTCGAGGAC
CGAGTCAGGCAGCTGCATGAAGCCCACAGGGACTTTGGTCCAGCATCTCAGCACTTTCTTTCCACGTCTGTCCAG
GGTCCCTGGGAGAGAGCCATCTCGCCAAACAAAGTGCCCTACTATATCAACCACGAGACTCAAACAACTTGCTGG
GACCATCCCAAAATGACAGAGCTCTACCAGTCTTTAGCTGACCTGAATAATGTCAGATTCTCAGCTTATAGGACT
GCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTAT
GACCGCCTGGAGCAAGAGCACAACAATTTGGTCAACGTCCCTCTCTGCGTGGATATGTGTCTGAACTGGCTGCTG
AATGTTTATGATACGGGACGAACAGGGAGGATCCGTGTCCTGTCTTTTAAAACTGGCATCATTTCCCTGTGTAAA
GCACATTTGGAAGACAAGTACAGATACCTTTTCAAGCAAGTGGCAAGTTCAACAGGATTTTGTGACCAGCGCAGG
CTGGGCCTCCTTCTGCATGATTCTATCCAAATTCCAAGACAGTTGGGTGAAGTTGCATCCTTTGGGGGCAGTAAC
ATTGAGCCAAGTGTCCGGAGCTGCTTCCAATTTGCTAATAATAAGCCAGAGATCGAAGCGGCCCTCTTCCTAGAC
TGGATGAGACTGGAACCCCAGTCCATGGTGTGGCTGCCCGTCCTGCACAGAGTGGCTGCTGCAGAAACTGCCAAG
CATCAGGCCAAATGTAACATCTGCAAAGAGTGTCCAATCATTGGATTCAGGTACAGGAGTCTAAAGCACTTTAAT
TATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAA
AGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACT
CATTCACGCATTGAACATTATGCTAGCAGGCTAGCAGAAATGGAAAACAGCAATGGATCTTATCTAAATGATAGC
ATCTCTCCTAATGAGAGCATAGATGATGAACATTTGTTAATCCAGCATTACTGCCAAAGTTTGAACCAGGACTCC
CCCCTGAGCCAGCCTCGTAGT
Human sequences with codon optimization:
(SEQ ID NO: 138)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGCCGCTAGCGCCCAGAGAAAAATAGATGAGACCCTT
GAAAGACTCCGGGAACTTCAAGAGGCCACGGATGAGCTGGACCTCAAGCTGCGCCAAGCTGAGGTGATCAAGGGA
TCCTGGCAGCCCGTGGGCGATCTCCTCATTGACTCTCTCCAAGATCACCTCGAGAAAGTCAAGGCACTTCGAGGA
GAAATTGCGCCTCTGAAAGAGAACGTGAGCCACGTCAATGACCTTGCTCGCCAGCTTACCACTTTGGGCATTCAG
CTCTCACCGTATAACCTCAGCACTCTGGAAGACCTGAACACCAGATGGAAGCTTCTGCAGGTGGCCGTCGAGGAC
CGAGTCAGGCAGCTGCATGAAGCCCACAGGGACTTTGGTCCAGCATCTCAGCACTTTCTTTCCACGTCTGTCCAG
GGTCCCTGGGAGAGAGCCATCTCGCCAAACAAAGTGCCCTACTATATCAACCACGAGACTCAAACAACTTGCTGG
GACCATCCCAAAATGACAGAGCTCTACCAGTCTTTAGCTGACCTGAATAATGTCAGATTCTCAGCTTATAGGACT
GCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTAT
GACCGCCTGGAGCAAGAGCACAACAATTTGGTCAACGTCCCTCTCTGCGTGGATATGTGTCTGAACTGGCTGCTG
AATGTTTATGATACGGGACGAACAGGGAGGATCCGTGTCCTGTCTTTTAAAACTGGCATCATTTCCCTGTGTAAA
GCACATTTGGAAGACAAGTACAGATACCTTTTCAAGCAAGTGGCAAGTTCAACAGGATTTTGTGACCAGCGCAGG
CTGGGCCTCCTTCTGCATGATTCTATCCAAATTCCAAGACAGTTGGGTGAAGTTGCATCCTTTGGGGGCAGTAAC
ATTGAGCCAAGTGTCCGGAGCTGCTTCCAATTTGCTAATAATAAGCCAGAGATCGAAGCGGCCCTCTTCCTAGAC
TGGATGAGACTGGAACCCCAGTCCATGGTGTGGCTGCCCGTCCTGCACAGAGTGGCTGCTGCAGAAACTGCCAAG
CATCAGGCCAAATGTAACATCTGCAAAGAGTGTCCAATCATTGGATTCAGGTACAGGAGTCTAAAGCACTTTAAT
TATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAA
AGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACT
CATTCACGCATTGAACATTATGCTAGCAGGCTAGCAGAAATGGAAAACAGCAATGGATCTTATCTAAATGATAGC
ATCTCTCCTAATGAGAGCATAGATGATGAACATTTGTTAATCCAGCATTACTGCCAAAGTTTGAACCAGGACTCC
CCCCTGAGCCAGCCTCGTAGT
Utrophin N-terminus through R3_Dystrophin R24 + H4 to 1st coiled coil and proline
rich region that follows:
Human sequences without codon alterations:
(SEQ ID NO: 139)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCtGCCGCTAGCGCCCAGAGAAAAATAGATGAGACCCTT
GAAAGACTCCGGGAACTTCAAGAGGCCACGGATGAGCTGGACCTCAAGCTGCGCCAAGCTGAGGTGATCAAGGGA
TCCTGGCAGCCCGTGGGCGATCTCCTCATTGACTCTCTCCAAGATCACCTCGAGAAAGTCAAGGCACTTCGAGGA
GAAATTGCGCCTCTGAAAGAGAACGTGAGCCACGTCAATGACCTTGCTCGCCAGCTTACCACTTTGGGCATTCAG
CTCTCACCGTATAACCTCAGCACTCTGGAAGACCTGAACACCAGATGGAAGCTTCTGCAGGTGGCCGTCGAGGAC
CGAGTCAGGCAGCTGCATGAAGCCCACAGGGACTTTGGTCCAGCATCTCAGCACTTTCTTTCCACGTCTGTCCAG
GGTCCCTGGGAGAGAGCCATCTCGCCAAACAAAGTGCCCTACTATATCAACCACGAGACTCAAACAACTTGCTGG
GACCATCCCAAAATGACAGAGCTCTACCAGTCTTTAGCTGACCTGAATAATGTCAGATTCTCAGCTTATAGGACT
GCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTAT
GACCGCCTGGAGCAAGAGCACAACAATTTGGTCAACGTCCCTCTCTGCGTGGATATGTGTCTGAACTGGCTGCTG
AATGTTTATGATACGGGACGAACAGGGAGGATCCGTGTCCTGTCTTTTAAAACTGGCATCATTTCCCTGTGTAAA
GCACATTTGGAAGACAAGTACAGATACCTTTTCAAGCAAGTGGCAAGTTCAACAGGATTTTGTGACCAGCGCAGG
CTGGGCCTCCTTCTGCATGATTCTATCCAAATTCCAAGACAGTTGGGTGAAGTTGCATCCTTTGGGGGCAGTAAC
ATTGAGCCAAGTGTCCGGAGCTGCTTCCAATTTGCTAATAATAAGCCAGAGATCGAAGCGGCCCTCTTCCTAGAC
TGGATGAGACTGGAACCCCAGTCCATGGTGTGGCTGCCCGTCCTGCACAGAGTGGCTGCTGCAGAAACTGCCAAG
CATCAGGCCAAATGTAACATCTGCAAAGAGTGTCCAATCATTGGATTCAGGTACAGGAGTCTAAAGCACTTTAAT
TATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAA
AGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACT
CATTCACGCATTGAACATTATGCTAGCAGGCTAGCAGAAATGGAAAACAGCAATGGATCTTATCTAAATGATAGC
ATCTCTCCTAATGAGAGCATAGATGATGAACATTTGTTAATCCAGCATTACTGCCAAAGTTTGAACCAGGACTCC
CCCCTGAGCCAGCCTCGTAGTCCTGCCCAGATCTTGATTTCCTTAGAGAGTGAGGAAAGAGGGGAGCTAGAGAGA
ATCCTAGCAGATCTTGAGGAAGAAAACAGGAATCTGCAAGCAGAATATGACCGTCTAAAGCAGCAGCACGAACAT
AAAGGCCTGTCCCCACTGCCGTCCCCTCCTGAAATGATGCCCACCTCTCCCCAGAGTCCC
Human sequences with codon optimization:
(SEQ ID NO: 140)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGCCGCTAGCGCCCAGAGAAAAATAGATGAGACCCTT
GAAAGACTCCGGGAACTTCAAGAGGCCACGGATGAGCTGGACCTCAAGCTGCGCCAAGCTGAGGTGATCAAGGGA
TCCTGGCAGCCCGTGGGCGATCTCCTCATTGACTCTCTCCAAGATCACCTCGAGAAAGTCAAGGCACTTCGAGGA
GAAATTGCGCCTCTGAAAGAGAACGTGAGCCACGTCAATGACCTTGCTCGCCAGCTTACCACTTTGGGCATTCAG
CTCTCACCGTATAACCTCAGCACTCTGGAAGACCTGAACACCAGATGGAAGCTTCTGCAGGTGGCCGTCGAGGAC
CGAGTCAGGCAGCTGCATGAAGCCCACAGGGACTTTGGTCCAGCATCTCAGCACTTTCTTTCCACGTCTGTCCAG
GGTCCCTGGGAGAGAGCCATCTCGCCAAACAAAGTGCCCTACTATATCAACCACGAGACTCAAACAACTTGCTGG
GACCATCCCAAAATGACAGAGCTCTACCAGTCTTTAGCTGACCTGAATAATGTCAGATTCTCAGCTTATAGGACT
GCCATGAAACTCCGAAGACTGCAGAAGGCCCTTTGCTTGGATCTCTTGAGCCTGTCAGCTGCATGTGATGCCTTG
GACCAGCACAACCTCAAGCAAAATGACCAGCCCATGGATATCCTGCAGATTATTAATTGTTTGACCACTATTTAT
GACCGCCTGGAGCAAGAGCACAACAATTTGGTCAACGTCCCTCTCTGCGTGGATATGTGTCTGAACTGGCTGCTG
AATGTTTATGATACGGGACGAACAGGGAGGATCCGTGTCCTGTCTTTTAAAACTGGCATCATTTCCCTGTGTAAA
GCACATTTGGAAGACAAGTACAGATACCTTTTCAAGCAAGTGGCAAGTTCAACAGGATTTTGTGACCAGCGCAGG
CTGGGCCTCCTTCTGCATGATTCTATCCAAATTCCAAGACAGTTGGGTGAAGTTGCATCCTTTGGGGGCAGTAAC
ATTGAGCCAAGTGTCCGGAGCTGCTTCCAATTTGCTAATAATAAGCCAGAGATCGAAGCGGCCCTCTTCCTAGAC
TGGATGAGACTGGAACCCCAGTCCATGGTGTGGCTGCCCGTCCTGCACAGAGTGGCTGCTGCAGAAACTGCCAAG
CATCAGGCCAAATGTAACATCTGCAAAGAGTGTCCAATCATTGGATTCAGGTACAGGAGTCTAAAGCACTTTAAT
TATGACATCTGCCAAAGCTGCTTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACTAAAAAACAAATTTCGAACCAAA
AGGTATTTTGCGAAGCATCCCCGAATGGGCTACCTGCCAGTGCAGACTGTCTTAGAGGGGGACAACATGGAAACT
CCCGTTACTCTGATCAACTTCTGGCCAGTAGATTCTGCGCCTGCCTCGTCCCCTCAGCTTTCACACGATGATACT
CATTCACGCATTGAACATTATGCTAGCAGGCTAGCAGAAATGGAAAACAGCAATGGATCTTATCTAAATGATAGC
ATCTCTCCTAATGAGAGCATAGATGATGAACATTTGTTAATCCAGCATTACTGCCAAAGTTTGAACCAGGACTCC
CCCCTGAGCCAGCCTCGTAGTCCTGCCCAGATCTTGATTTCCTTAGAGAGTGAGGAAAGAGGGGAGCTAGAGAGA
ATCCTAGCAGATCTTGAGGAAGAAAACAGGAATCTGCAAGCAGAATATGACCGTCTAAAGCAGCAGCACGAACAT
AAAGGCCTGTCCCCACTGCCGTCCCCTCCTGAAATGATGCCCACCTCTCCCCAGAGTCCC
Utrophin N-terminus through H2_Dystrophin R24 + H4 to end of syntrophin region:
Human sequences without codon alterations:
(SEQ ID NO: 141)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCTGTAGCAAAGCTGGGGATGTCTCAGATTCCTCAGAAG
GACCTTTTGGAGACTGTTCGTGTAAGAGAACAAGCAATTACAAAAAAATCTAAGCAGGAAGCCGCTAGCGCCCAG
AGAAAAATAGATGAGACCCTTGAAAGACTCCGGGAACTTCAAGAGGCCACGGATGAGCTGGACCTCAAGCTGCGC
CAAGCTGAGGTGATCAAGGGATCCTGGCAGCCCGTGGGCGATCTCCTCATTGACTCTCTCCAAGATCACCTCGAG
AAAGTCAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGAAGCTT
CTGCAGGTGGCCGTCGAGGACCGAGTCAGGCAGCTGCATGAAGCCCACAGGGACTTTGGTCCAGCATCTCAGCAC
TTTCTTTCCACGTCTGTCCAGGGTCCCTGGGAGAGAGCCATCTCGCCAAACAAAGTGCCCTACTATATCAACCAC
GAGACTCAAACAACTTGCTGGGACCATCCCAAAATGACAGAGCTCTACCAGTCTTTAGCTGACCTGAATAATGTC
AGATTCTCAGCTTATAGGACTGCCATGAAACTCCGAAGACTGCAGAAGGCCCTTTGCTTGGATCTCTTGAGCCTG
TCAGCTGCATGTGATGCCTTGGACCAGCACAACCTCAAGCAAAATGACCAGCCCATGGATATCCTGCAGATTATT
AATTGTTTGACCACTATTTATGACCGCCTGGAGCAAGAGCACAACAATTTGGTCAACGTCCCTCTCTGCGTGGAT
ATGTGTCTGAACTGGCTGCTGAATGTTTATGATACGGGACGAACAGGGAGGATCCGTGTCCTGTCTTTTAAAACT
GGCATCATTTCCCTGTGTAAAGCACATTTGGAAGACAAGTACAGATACCTTTTCAAGCAAGTGGCAAGTTCAACA
GGATTTTGTGACCAGCGCAGGCTGGGCCTCCTTCTGCATGATTCTATCCAAATTCCAAGACAGTTGGGTGAAGTT
GCATCCTTTGGGGGCAGTAACATTGAGCCAAGTGTCCGGAGCTGCTTCCAATTTGCTAATAATAAGCCAGAGATC
GAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTAC
AGGAGTCTAAAGCACTTTAATTATGACATCTGCCAAAGCTGCTTTTTTTCTGGTCGAGTTGCAAAAGGCCATAAA
ATGCACTATCCCATGGTGGAATATTGCACTCCGACTACATCAGGAGAAGATGTTCGAGACTTTGCCAAGGTACTA
AAAAACAAATTTCGAACCAAAAGGTATTTTGCGAAGCATCCCCGAATGGGCTACCTGCCAGTGCAGACTGTCTTA
GAGGGGGACAACATGGAAACTCCCGTTACTCTGATCAACTTCTGGCCAGTAGATTCTGCGCCTGCCTCGTCCCCT
CAGCTTTCACACGATGATACTCATTCACGCATTGAACATTATGCTAGCAGGCTAGCAGAAATGGAAAACAGCAAT
GGATCTTATCTAAATGATAGCATCTCTCCTAATGAGAGCATAGATGATGAACATTTGTTAATCCAGCATTACTGC
CAAAGTTTGAACCAGGACTCCCCCCTGAGCCAGCCTCGTAGT
Human sequences with codon optimization:
(SEQ ID NO: 142)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGTGGCCAAGCTGGGCATGAGCCAGATCCCCCAGAAG
GACCTGCTGGAGACCGTGCGGGTGCGGGAGCAGGCCATCACCAAGAAGAGCAAGCAGGAGGCCGCTAGCGCCCAG
AGAAAAATAGATGAGACCCTTGAAAGACTCCGGGAACTTCAAGAGGCCACGGATGAGCTGGACCTCAAGCTGCGC
CAAGCTGAGGTGATCAAGGGATCCTGGCAGCCCGTGGGCGATCTCCTCATTGACTCTCTCCAAGATCACCTCGAG
AAAGTCAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGAAGCTT
CTGCAGGTGGCCGTCGAGGACCGAGTCAGGCAGCTGCATGAAGCCCACAGGGACTTTGGTCCAGCATCTCAGCAC
TTTCTTTCCACGTCTGTCCAGGGTCCCTGGGAGAGAGCCATCTCGCCAAACAAAGTGCCCTACTATATCAACCAC
GAGACTCAAACAACTTGCTGGGACCATCCCAAAATGACAGAGCTCTACCAGTCTTTAGCTGACCTGAATAATGTC
AGATTCTCAGCTTATAGGACTGCCATGAAACTCCGAAGACTGCAGAAGGCCCTTTGCTTGGATCTCTTGAGCCTG
TCAGCTGCATGTGATGCCTTGGACCAGCACAACCTCAAGCAAAATGACCAGCCCATGGATATCCTGCAGATTATT
AATTGTTTGACCACTATTTATGACCGCCTGGAGCAAGAGCACAACAATTTGGTCAACGTCCCTCTCTGCGTGGAT
ATGTGTCTGAACTGGCTGCTGAATGTTTATGATACGGGACGAACAGGGAGGATCCGTGTCCTGTCTTTTAAAACT
GGCATCATTTCCCTGTGTAAAGCACATTTGGAAGACAAGTACAGATACCTTTTCAAGCAAGTGGCAAGTTCAACA
GGATTTTGTGACCAGCGCAGGCTGGGCCTCCTTCTGCATGATTCTATCCAAATTCCAAGACAGTTGGGTGAAGTT
GCATCCTTTGGGGGCAGTAACATTGAGCCAAGTGTCCGGAGCTGCTTCCAATTTGCTAATAATAAGCCAGAGATC
GAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTAC
AGGAGTCTAAAGCACTTTAATTATGACATCTGCCAAAGCTGCTTTTTTTCTGGTCGAGTTGCAAAAGGCCATAAA
ATGCACTATCCCATGGTGGAATATTGCACTCCGACTACATCAGGAGAAGATGTTCGAGACTTTGCCAAGGTACTA
AAAAACAAATTTCGAACCAAAAGGTATTTTGCGAAGCATCCCCGAATGGGCTACCTGCCAGTGCAGACTGTCTTA
GAGGGGGACAACATGGAAACTCCCGTTACTCTGATCAACTTCTGGCCAGTAGATTCTGCGCCTGCCTCGTCCCCT
CAGCTTTCACACGATGATACTCATTCACGCATTGAACATTATGCTAGCAGGCTAGCAGAAATGGAAAACAGCAAT
GGATCTTATCTAAATGATAGCATCTCTCCTAATGAGAGCATAGATGATGAACATTTGTTAATCCAGCATTACTGC
CAAAGTTTGAACCAGGACTCCCCCCTGAGCCAGCCTCGTAGT
Utrophin N-terminus through H2_Dystrophin R24 to end:
Human sequences without codon alterations:
(SEQ ID NO: 143)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCTGTAGCAAAGCTGGGGATGTCTCAGATTCCTCAGAAG
GACCTTTTGGAGACTGTTCGTGTAAGAGAACAAGCAATTACAAAAAAATCTAAGCAGGAAGCCGCTAGCGCCCAG
AGAAAAATAGATGAGACCCTTGAAAGACTCCGGGAACTTCAAGAGGCCACGGATGAGCTGGACCTCAAGCTGCGC
CAAGCTGAGGTGATCAAGGGATCCTGGCAGCCCGTGGGCGATCTCCTCATTGACTCTCTCCAAGATCACCTCGAG
AAAGTCAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGAAGCTT
CTGCAGGTGGCCGTCGAGGACCGAGTCAGGCAGCTGCATGAAGCCCACAGGGACTTTGGTCCAGCATCTCAGCAC
TTTCTTTCCACGTCTGTCCAGGGTCCCTGGGAGAGAGCCATCTCGCCAAACAAAGTGCCCTACTATATCAACCAC
GAGACTCAAACAACTTGCTGGGACCATCCCAAAATGACAGAGCTCTACCAGTCTTTAGCTGACCTGAATAATGTC
AGATTCTCAGCTTATAGGACTGCCATGAAACTCCGAAGACTGCAGAAGGCCCTTTGCTTGGATCTCTTGAGCCTG
TCAGCTGCATGTGATGCCTTGGACCAGCACAACCTCAAGCAAAATGACCAGCCCATGGATATCCTGCAGATTATT
AATTGTTTGACCACTATTTATGACCGCCTGGAGCAAGAGCACAACAATTTGGTCAACGTCCCTCTCTGCGTGGAT
ATGTGTCTGAACTGGCTGCTGAATGTTTATGATACGGGACGAACAGGGAGGATCCGTGTCCTGTCTTTTAAAACT
GGCATCATTTCCCTGTGTAAAGCACATTTGGAAGACAAGTACAGATACCTTTTCAAGCAAGTGGCAAGTTCAACA
GGATTTTGTGACCAGCGCAGGCTGGGCCTCCTTCTGCATGATTCTATCCAAATTCCAAGACAGTTGGGTGAAGTT
GCATCCTTTGGGGGCAGTAACATTGAGCCAAGTGTCCGGAGCTGCTTCCAATTTGCTAATAATAAGCCAGAGATC
GAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTAC
AGGAGTCTAAAGCACTTTAATTATGACATCTGCCAAAGCTGCTTTTTTTCTGGTCGAGTTGCAAAAGGCCATAAA
ATGCACTATCCCATGGTGGAATATTGCACTCCGACTACATCAGGAGAAGATGTTCGAGACTTTGCCAAGGTACTA
AAAAACAAATTTCGAACCAAAAGGTATTTTGCGAAGCATCCCCGAATGGGCTACCTGCCAGTGCAGACTGTCTTA
GAGGGGGACAACATGGAAACTCCCGTTACTCTGATCAACTTCTGGCCAGTAGATTCTGCGCCTGCCTCGTCCCCT
CAGCTTTCACACGATGATACTCATTCACGCATTGAACATTATGCTAGCAGGCTAGCAGAAATGGAAAACAGCAAT
GGATCTTATCTAAATGATAGCATCTCTCCTAATGAGAGCATAGATGATGAACATTTGTTAATCCAGCATTACTGC
CAAAGTTTGAACCAGGACTCCCCCCTGAGCCAGCCTCGTAGTCCTGCCCAGATCTTGATTTCCTTAGAGAGTGAG
GAAAGAGGGGAGCTAGAGAGAATCCTAGCAGATCTTGAGGAAGAAAACAGGAATCTGCAAGCAGAATATGACCGT
CTAAAGCAGCAGCACGAACATAAAGGCCTGTCCCCACTGCCGTCCCCTCCTGAAATGATGCCCACCTCTCCCCAG
AGTCCCCGGGATGCTGAGCTCATTGCTGAGGCCAAGCTACTGCGTCAACACAAAGGCCGCCTGGAAGCCAGGATG
CAAATCCTGGAAGACCACAATAAACAGCTGGAGTCACAGTTACACAGGCTAAGGCAGCTGCTGGAGCAACCCCAG
GCAGAGGCCAAAGTGAATGGCACAACGGTGTCCTCTCCTTCTACCTCTCTACAGAGGTCCGACAGCAGTCAGCCT
ATGCTGCTCCGAGTGGTTGGCAGTCAAACTTCGGACTCCATGGGTGAGGAAGATCTTCTCAGTCCTCCCCAGGAC
ACAAGCACAGGGTTAGAGGAGGTGATGGAGCAACTCAACAACTCCTTCCCTAGTTCAAGAGGAAGAAATACCCCT
GGAAAGCCAATGAGAGAGGACACAATG
Human sequences with codon optimization:
(SEQ ID NO: 144)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGTGGCCAAGCTGGGCATGAGCCAGATCCCCCAGAAG
GACCTGCTGGAGACCGTGCGGGTGCGGGAGCAGGCCATCACCAAGAAGAGCAAGCAGGAGGCCGCTAGCGCCCAG
AGAAAAATAGATGAGACCCTTGAAAGACTCCGGGAACTTCAAGAGGCCACGGATGAGCTGGACCTCAAGCTGCGC
CAAGCTGAGGTGATCAAGGGATCCTGGCAGCCCGTGGGCGATCTCCTCATTGACTCTCTCCAAGATCACCTCGAG
AAAGTCAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGAAGCTT
CTGCAGGTGGCCGTCGAGGACCGAGTCAGGCAGCTGCATGAAGCCCACAGGGACTTTGGTCCAGCATCTCAGCAC
TTTCTTTCCACGTCTGTCCAGGGTCCCTGGGAGAGAGCCATCTCGCCAAACAAAGTGCCCTACTATATCAACCAC
GAGACTCAAACAACTTGCTGGGACCATCCCAAAATGACAGAGCTCTACCAGTCTTTAGCTGACCTGAATAATGTC
AGATTCTCAGCTTATAGGACTGCCATGAAACTCCGAAGACTGCAGAAGGCCCTTTGCTTGGATCTCTTGAGCCTG
TCAGCTGCATGTGATGCCTTGGACCAGCACAACCTCAAGCAAAATGACCAGCCCATGGATATCCTGCAGATTATT
AATTGTTTGACCACTATTTATGACCGCCTGGAGCAAGAGCACAACAATTTGGTCAACGTCCCTCTCTGCGTGGAT
ATGTGTCTGAACTGGCTGCTGAATGTTTATGATACGGGACGAACAGGGAGGATCCGTGTCCTGTCTTTTAAAACT
GGCATCATTTCCCTGTGTAAAGCACATTTGGAAGACAAGTACAGATACCTTTTCAAGCAAGTGGCAAGTTCAACA
GGATTTTGTGACCAGCGCAGGCTGGGCCTCCTTCTGCATGATTCTATCCAAATTCCAAGACAGTTGGGTGAAGTT
GCATCCTTTGGGGGCAGTAACATTGAGCCAAGTGTCCGGAGCTGCTTCCAATTTGCTAATAATAAGCCAGAGATC
GAAGCGGCCCTCTTCCTAGACTGGATGAGACTGGAACCCCAGTCCATGGTGTGGCTGCCCGTCCTGCACAGAGTG
GCTGCTGCAGAAACTGCCAAGCATCAGGCCAAATGTAACATCTGCAAAGAGTGTCCAATCATTGGATTCAGGTAC
AGGAGTCTAAAGCACTTTAATTATGACATCTGCCAAAGCTGCTTTTTTTCTGGTCGAGTTGCAAAAGGCCATAAA
ATGCACTATCCCATGGTGGAATATTGCACTCCGACTACATCAGGAGAAGATGTTCGAGACTTTGCCAAGGTACTA
AAAAACAAATTTCGAACCAAAAGGTATTTTGCGAAGCATCCCCGAATGGGCTACCTGCCAGTGCAGACTGTCTTA
GAGGGGGACAACATGGAAACTCCCGTTACTCTGATCAACTTCTGGCCAGTAGATTCTGCGCCTGCCTCGTCCCCT
CAGCTTTCACACGATGATACTCATTCACGCATTGAACATTATGCTAGCAGGCTAGCAGAAATGGAAAACAGCAAT
GGATCTTATCTAAATGATAGCATCTCTCCTAATGAGAGCATAGATGATGAACATTTGTTAATCCAGCATTACTGC
CAAAGTTTGAACCAGGACTCCCCCCTGAGCCAGCCTCGTAGTCCTGCCCAGATCTTGATTTCCTTAGAGAGTGAG
GAAAGAGGGGAGCTAGAGAGAATCCTAGCAGATCTTGAGGAAGAAAACAGGAATCTGCAAGCAGAATATGACCGT
CTAAAGCAGCAGCACGAACATAAAGGCCTGTCCCCACTGCCGTCCCCTCCTGAAATGATGCCCACCTCTCCCCAG
AGTCCCCGGGATGCTGAGCTCATTGCTGAGGCCAAGCTACTGCGTCAACACAAAGGCCGCCTGGAAGCCAGGATG
CAAATCCTGGAAGACCACAATAAACAGCTGGAGTCACAGTTACACAGGCTAAGGCAGCTGCTGGAGCAACCCCAG
GCAGAGGCCAAAGTGAATGGCACAACGGTGTCCTCTCCTTCTACCTCTCTACAGAGGTCCGACAGCAGTCAGCCT
ATGCTGCTCCGAGTGGTTGGCAGTCAAACTTCGGACTCCATGGGTGAGGAAGATCTTCTCAGTCCTCCCCAGGAC
ACAAGCACAGGGTTAGAGGAGGTGATGGAGCAACTCAACAACTCCTTCCCTAGTTCAAGAGGAAGAAATACCCCT
GGAAAGCCAATGAGAGAGGACACAATG
Utrophin N-terminus through R3_Dystrophin R23 to end:
Human sequences without codon alterations:
(SEQ ID NO: 145)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCTGCCGCTAGCGCCAGTTCTGACCAGTGGAAGCGTCTG
CACCTTTCTCTGCAGGAACTTCTGGTGTGGCTACAGCTGAAAGATGATGAATTAAGCCGGCAGGCACCTATTGGA
GGCGACTTTCCAGCAGTTCAGAAGCAGAACGATGTACATAGGGCCTTCAAGAGGGAATTGAAAACTAAAGAACCT
GTAATCATGAGTACTCTTGAGACTGTACGAATATTTCTGACAGAGCAGCCTTTGGAAGGACTAGAGAAACTCTAC
CAGGAGCCCAGAGAGCTGCCTCCTGAGGAGAGAGCCCAGAATGTCACTCGGCTTCTACGAAAGCAGGCTGAGGAG
GTCAATACTGAGTGGGAAAAATTGAACCTGCACTCCGCTGACCAGAGAAAAATAGATGAGACCCTTGAAAGACTC
CGGGAACTTCAAGAGGCCACGGATGAGCTGGACCTCAAGCTGCGCCAAGCTGAGGTGATCAAGGGATCCTGGCAG
CCCGTGGGCGATCTCCTCATTGACTCTCTCCAAGATCACCTCGAGAAAGTCAAGGCACTTCGAGGAGAAATTGCG
CCTCTGAAAGAGAACGTGAGCCACGTCAATGACCTTGCTCGCCAGCTTACCACTTTGGGCATTCAGCTCTCACCG
TATAACCTCAGCACTCTGGAAGACCTGAACACCAGATGGAAGCTTCTGCAGGTGGCCGTCGAGGACCGAGTCAGG
CAGCTGCATGAAGCCCACAGGGACTTTGGTCCAGCATCTCAGCACTTTCTTTCCACGTCTGTCCAGGGTCCCTGG
GAGAGAGCCATCTCGCCAAACAAAGTGCCCTACTATATCAACCACGAGACTCAAACAACTTGCTGGGACCATCCC
AAAATGACAGAGCTCTACCAGTCTTTAGCTGACCTGAATAATGTCAGATTCTCAGCTTATAGGACTGCCATGAAA
CTCCGAAGACTGCAGAAGGCCCTTTGCTTGGATCTCTTGAGCCTGTCAGCTGCATGTGATGCCTTGGACCAGCAC
AACCTCAAGCAAAATGACCAGCCCATGGATATCCTGCAGATTATTAATTGTTTGACCACTATTTATGACCGCCTG
GAGCAAGAGCACAACAATTTGGTCAACGTCCCTCTCTGCGTGGATATGTGTCTGAACTGGCTGCTGAATGTTTAT
GATACGGGACGAACAGGGAGGATCCGTGTCCTGTCTTTTAAAACTGGCATCATTTCCCTGTGTAAAGCACATTTG
GAAGACAAGTACAGATACCTTTTCAAGCAAGTGGCAAGTTCAACAGGATTTTGTGACCAGCGCAGGCTGGGCCTC
CTTCTGCATGATTCTATCCAAATTCCAAGACAGTTGGGTGAAGTTGCATCCTTTGGGGGCAGTAACATTGAGCCA
AGTGTCCGGAGCTGCTTCCAATTTGCTAATAATAAGCCAGAGATCGAAGCGGCCCTCTTCCTAGACTGGATGAGA
CTGGAACCCCAGTCCATGGTGTGGCTGCCCGTCCTGCACAGAGTGGCTGCTGCAGAAACTGCCAAGCATCAGGCC
AAATGTAACATCTGCAAAGAGTGTCCAATCATTGGATTCAGGTACAGGAGTCTAAAGCACTTTAATTATGACATC
TGCCAAAGCTGCTTTTTTTCTGGTCGAGTTGCAAAAGGCCATAAAATGCACTATCCCATGGTGGAATATTGCACT
CCGACTACATCAGGAGAAGATGTTCGAGACTTTGCCAAGGTACTAAAAAACAAATTTCGAACCAAAAGGTATTTT
GCGAAGCATCCCCGAATGGGCTACCTGCCAGTGCAGACTGTCTTAGAGGGGGACAACATGGAAACTCCCGTTACT
CTGATCAACTTCTGGCCAGTAGATTCTGCGCCTGCCTCGTCCCCTCAGCTTTCACACGATGATACTCATTCACGC
ATTGAACATTATGCTAGCAGGCTAGCAGAAATGGAAAACAGCAATGGATCTTATCTAAATGATAGCATCTCTCCT
AATGAGAGCATAGATGATGAACATTTGTTAATCCAGCATTACTGCCAAAGTTTGAACCAGGACTCCCCCCTGAGC
CAGCCTCGTAGTCCTGCCCAGATCTTGATTTCCTTAGAGAGTGAGGAAAGAGGGGAGCTAGAGAGAATCCTAGCA
GATCTTGAGGAAGAAAACAGGAATCTGCAAGCAGAATATGACCGTCTAAAGCAGCAGCACGAACATAAAGGCCTG
TCCCCACTGCCGTCCCCTCCTGAAATGATGCCCACCTCTCCCCAGAGTCCCCGGGATGCTGAGCTCATTGCTGAG
GCCAAGCTACTGCGTCAACACAAAGGCCGCCTGGAAGCCAGGATGCAAATCCTGGAAGACCACAATAAACAGCTG
GAGTCACAGTTACACAGGCTAAGGCAGCTGCTGGAGCAACCCCAGGCAGAGGCCAAAGTGAATGGCACAACGGTG
TCCTCTCCTTCTACCTCTCTACAGAGGTCCGACAGCAGTCAGCCTATGCTGCTCCGAGTGGTTGGCAGTCAAACT
TCGGACTCCATGGGTGAGGAAGATCTTCTCAGTCCTCCCCAGGACACAAGCACAGGGTTAGAGGAGGTGATGGAG
CAACTCAACAACTCCTTCCCTAGTTCAAGAGGAAGAAATACCCCTGGAAAGCCAATGAGAGAGGACACAATG
Human sequences with codon optimization:
(SEQ ID NO: 146)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGCCGCTAGCGCCAGCAGCGACCAGTGGAAGCGGCTG
CACCTGAGCCTGCAGGAGCTGCTGGTGTGGCTGCAGCTGAAGGACGACGAGCTGAGCCGGCAGGCCCCCATCGGC
GGCGACTTCCCCGCCGTGCAGAAGCAGAACGACGTGCACCGGGCCTTCAAGCGGGAGCTGAAGACCAAGGAGCCC
GTGATCATGAGCACCCTGGAGACCGTGCGGATCTTCCTGACCGAGCAGCCCCTGGAGGGCCTGGAGAAGCTGTAC
CAGGAGCCCCGGGAGCTGCCCCCCGAGGAGCGGGCCCAGAACGTGACCCGGCTGCTGCGGAAGCAGGCCGAGGAG
GTGAACACCGAGTGGGAGAAGCTGAACCTGCACAGCGCCGACCAGAGAAAAATAGATGAGACCCTTGAAAGACTC
CGGGAACTTCAAGAGGCCACGGATGAGCTGGACCTCAAGCTGCGCCAAGCTGAGGTGATCAAGGGATCCTGGCAG
CCCGTGGGCGATCTCCTCATTGACTCTCTCCAAGATCACCTCGAGAAAGTCAAGGCACTTCGAGGAGAAATTGCG
CCTCTGAAAGAGAACGTGAGCCACGTCAATGACCTTGCTCGCCAGCTTACCACTTTGGGCATTCAGCTCTCACCG
TATAACCTCAGCACTCTGGAAGACCTGAACACCAGATGGAAGCTTCTGCAGGTGGCCGTCGAGGACCGAGTCAGG
CAGCTGCATGAAGCCCACAGGGACTTTGGTCCAGCATCTCAGCACTTTCTTTCCACGTCTGTCCAGGGTCCCTGG
GAGAGAGCCATCTCGCCAAACAAAGTGCCCTACTATATCAACCACGAGACTCAAACAACTTGCTGGGACCATCCC
AAAATGACAGAGCTCTACCAGTCTTTAGCTGACCTGAATAATGTCAGATTCTCAGCTTATAGGACTGCCATGAAA
CTCCGAAGACTGCAGAAGGCCCTTTGCTTGGATCTCTTGAGCCTGTCAGCTGCATGTGATGCCTTGGACCAGCAC
AACCTCAAGCAAAATGACCAGCCCATGGATATCCTGCAGATTATTAATTGTTTGACCACTATTTATGACCGCCTG
GAGCAAGAGCACAACAATTTGGTCAACGTCCCTCTCTGCGTGGATATGTGTCTGAACTGGCTGCTGAATGTTTAT
GATACGGGACGAACAGGGAGGATCCGTGTCCTGTCTTTTAAAACTGGCATCATTTCCCTGTGTAAAGCACATTTG
GAAGACAAGTACAGATACCTTTTCAAGCAAGTGGCAAGTTCAACAGGATTTTGTGACCAGCGCAGGCTGGGCCTC
CTTCTGCATGATTCTATCCAAATTCCAAGACAGTTGGGTGAAGTTGCATCCTTTGGGGGCAGTAACATTGAGCCA
AGTGTCCGGAGCTGCTTCCAATTTGCTAATAATAAGCCAGAGATCGAAGCGGCCCTCTTCCTAGACTGGATGAGA
CTGGAACCCCAGTCCATGGTGTGGCTGCCCGTCCTGCACAGAGTGGCTGCTGCAGAAACTGCCAAGCATCAGGCC
AAATGTAACATCTGCAAAGAGTGTCCAATCATTGGATTCAGGTACAGGAGTCTAAAGCACTTTAATTATGACATC
TGCCAAAGCTGCTTTTTTTCTGGTCGAGTTGCAAAAGGCCATAAAATGCACTATCCCATGGTGGAATATTGCACT
CCGACTACATCAGGAGAAGATGTTCGAGACTTTGCCAAGGTACTAAAAAACAAATTTCGAACCAAAAGGTATTTT
GCGAAGCATCCCCGAATGGGCTACCTGCCAGTGCAGACTGTCTTAGAGGGGGACAACATGGAAACTCCCGTTACT
CTGATCAACTTCTGGCCAGTAGATTCTGCGCCTGCCTCGTCCCCTCAGCTTTCACACGATGATACTCATTCACGC
ATTGAACATTATGCTAGCAGGCTAGCAGAAATGGAAAACAGCAATGGATCTTATCTAAATGATAGCATCTCTCCT
AATGAGAGCATAGATGATGAACATTTGTTAATCCAGCATTACTGCCAAAGTTTGAACCAGGACTCCCCCCTGAGC
CAGCCTCGTAGTCCTGCCCAGATCTTGATTTCCTTAGAGAGTGAGGAAAGAGGGGAGCTAGAGAGAATCCTAGCA
GATCTTGAGGAAGAAAACAGGAATCTGCAAGCAGAATATGACCGTCTAAAGCAGCAGCACGAACATAAAGGCCTG
TCCCCACTGCCGTCCCCTCCTGAAATGATGCCCACCTCTCCCCAGAGTCCCCGGGATGCTGAGCTCATTGCTGAG
GCCAAGCTACTGCGTCAACACAAAGGCCGCCTGGAAGCCAGGATGCAAATCCTGGAAGACCACAATAAACAGCTG
GAGTCACAGTTACACAGGCTAAGGCAGCTGCTGGAGCAACCCCAGGCAGAGGCCAAAGTGAATGGCACAACGGTG
TCCTCTCCTTCTACCTCTCTACAGAGGTCCGACAGCAGTCAGCCTATGCTGCTCCGAGTGGTTGGCAGTCAAACT
TCGGACTCCATGGGTGAGGAAGATCTTCTCAGTCCTCCCCAGGACACAAGCACAGGGTTAGAGGAGGTGATGGAG
CAACTCAACAACTCCTTCCCTAGTTCAAGAGGAAGAAATACCCCTGGAAAGCCAATGAGAGAGGACACAATG
Utrophin N-terminus through R3_Dystrophin R23 + H4 to 1st coiled coil and proline
rich region that follows:
Human sequences without codon alterations:
(SEQ ID NO: 147)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGATCTGGACAGCTATCAGATTGCGTTGGAGGAAGTGCTGACCTGGTTG
CTTTCTGCTGAGGACACTTTCCAGGAGCAGGATGATATTTCTGATGATGTTGAAGAAGTCAAAGACCAGTTTGCA
ACCCATGAAGCTTTTATGATGGAACTGACTGCACACCAGAGCAGTGTGGGCAGCGTCCTGCAGGCAGGCAACCAA
CTGATAACACAAGGAACTCTGTCAGACGAAGAAGAATTTGAGATTCAGGAACAGATGACCCTGCTGAATGCTAGA
TGGGAGGCTCTTAGGGTGGAGAGTATGGACAGACAGTCCCGGCTGCACGATGTGCTGATGGAACTGCAGAAGAAG
CAACTGCAGCAGCTCTCCGCCTGGTTAACACTCACAGAGGAGCGCATTCAGAAGATGGAAACTTGCCCCCTGGAT
GATGATGTAAAATCTCTACAAAAGCTGCTAGAAGAACATAAAAGTTTGCAAAGTGATCTTGAGGCTGAACAGGTG
AAAGTAAATTCACTAACTCACATGGTGGTCATTGTTGATGAAAACAGTGGTGAGAGTGCTACAGCTATCCTAGAA
GACCAGTTACAGAAACTTGGTGAGCGCTGGACAGCAGTATGCCGTTGGACTGAAGAACGCTGGAATAGGTTACAA
GAAATCAATATATTGTGGCAGGAATTATTGGAAGAACAGTGCTTGTTGAAAGCTTGGTTAACCGAAAAAGAAGAG
GCTTTAAATAAAGTCCAGACAAGCAACTTCAAAGACCAAAAGGAACTAAGTGTCAGTGTTCGACGTCTGGCTATT
TTGAAGGAAGACATGGAAATGAAGCGTCAAACATTGGATCAGCTGAGTGAGATTGGCCAGGATGTGGGACAATTA
CTTGATAATTCCAAGGCATCTAAGAAGATCAACAGTGACTCAGAGGAACTGACTCAAAGATGGGATTCTTTGGTT
CAGAGACTAGAAGATTCCTCCAACCAGGTGACTCAGGCTGCCGCTAGCGCCAGTTCTGACCAGTGGAAGCGTCTG
CACCTTTCTCTGCAGGAACTTCTGGTGTGGCTACAGCTGAAAGATGATGAATTAAGCCGGCAGGCACCTATTGGA
GGCGACTTTCCAGCAGTTCAGAAGCAGAACGATGTACATAGGGCCTTCAAGAGGGAATTGAAAACTAAAGAACCT
GTAATCATGAGTACTCTTGAGACTGTACGAATATTTCTGACAGAGCAGCCTTTGGAAGGACTAGAGAAACTCTAC
CAGGAGCCCAGAGAGCTGCCTCCTGAGGAGAGAGCCCAGAATGTCACTCGGCTTCTACGAAAGCAGGCTGAGGAG
GTCAATACTGAGTGGGAAAAATTGAACCTGCACTCCGCTGACCAGAGAAAAATAGATGAGACCCTTGAAAGACTC
CGGGAACTTCAAGAGGCCACGGATGAGCTGGACCTCAAGCTGCGCCAAGCTGAGGTGATCAAGGGATCCTGGCAG
CCCGTGGGCGATCTCCTCATTGACTCTCTCCAAGATCACCTCGAGAAAGTCAAGGCACTTCGAGGAGAAATTGCG
CCTCTGAAAGAGAACGTGAGCCACGTCAATGACCTTGCTCGCCAGCTTACCACTTTGGGCATTCAGCTCTCACCG
TATAACCTCAGCACTCTGGAAGACCTGAACACCAGATGGAAGCTTCTGCAGGTGGCCGTCGAGGACCGAGTCAGG
CAGCTGCATGAAGCCCACAGGGACTTTGGTCCAGCATCTCAGCACTTTCTTTCCACGTCTGTCCAGGGTCCCTGG
GAGAGAGCCATCTCGCCAAACAAAGTGCCCTACTATATCAACCACGAGACTCAAACAACTTGCTGGGACCATCCC
AAAATGACAGAGCTCTACCAGTCTTTAGCTGACCTGAATAATGTCAGATTCTCAGCTTATAGGACTGCCATGAAA
CTCCGAAGACTGCAGAAGGCCCTTTGCTTGGATCTCTTGAGCCTGTCAGCTGCATGTGATGCCTTGGACCAGCAC
AACCTCAAGCAAAATGACCAGCCCATGGATATCCTGCAGATTATTAATTGTTTGACCACTATTTATGACCGCCTG
GAGCAAGAGCACAACAATTTGGTCAACGTCCCTCTCTGCGTGGATATGTGTCTGAACTGGCTGCTGAATGTTTAT
GATACGGGACGAACAGGGAGGATCCGTGTCCTGTCTTTTAAAACTGGCATCATTTCCCTGTGTAAAGCACATTTG
GAAGACAAGTACAGATACCTTTTCAAGCAAGTGGCAAGTTCAACAGGATTTTGTGACCAGCGCAGGCTGGGCCTC
CTTCTGCATGATTCTATCCAAATTCCAAGACAGTTGGGTGAAGTTGCATCCTTTGGGGGCAGTAACATTGAGCCA
AGTGTCCGGAGCTGCTTCCAATTTGCTAATAATAAGCCAGAGATCGAAGCGGCCCTCTTCCTAGACTGGATGAGA
CTGGAACCCCAGTCCATGGTGTGGCTGCCCGTCCTGCACAGAGTGGCTGCTGCAGAAACTGCCAAGCATCAGGCC
AAATGTAACATCTGCAAAGAGTGTCCAATCATTGGATTCAGGTACAGGAGTCTAAAGCACTTTAATTATGACATC
TGCCAAAGCTGCTTTTTTTCTGGTCGAGTTGCAAAAGGCCATAAAATGCACTATCCCATGGTGGAATATTGCACT
CCGACTACATCAGGAGAAGATGTTCGAGACTTTGCCAAGGTACTAAAAAACAAATTTCGAACCAAAAGGTATTTT
GCGAAGCATCCCCGAATGGGCTACCTGCCAGTGCAGACTGTCTTAGAGGGGGACAACATGGAAACTCCCGTTACT
CTGATCAACTTCTGGCCAGTAGATTCTGCGCCTGCCTCGTCCCCTCAGCTTTCACACGATGATACTCATTCACGC
ATTGAACATTATGCTAGCAGGCTAGCAGAAATGGAAAACAGCAATGGATCTTATCTAAATGATAGCATCTCTCCT
AATGAGAGCATAGATGATGAACATTTGTTAATCCAGCATTACTGCCAAAGTTTGAACCAGGACTCCCCCCTGAGC
CAGCCTCGTAGTCCTGCCCAGATCTTGATTTCCTTAGAGAGTGAGGAAAGAGGGGAGCTAGAGAGAATCCTAGCA
GATCTTGAGGAAGAAAACAGGAATCTGCAAGCAGAATATGACCGTCTAAAGCAGCAGCACGAACATAAAGGCCTG
TCCCCACTGCCGTCCCCTCCTGAAATGATGCCCACCTCTCCCCAGAGTCCC
Human sequences with codon optimization:
(SEQ ID NO: 148)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGACCTGGACAGCTACCAGATCGCCCTGGAGGAGGTGCTGACCTGGCTG
CTGAGCGCCGAGGACACCTTCCAGGAGCAGGACGACATCAGCGACGACGTGGAGGAGGTGAAGGACCAGTTCGCC
ACCCACGAGGCCTTCATGATGGAGCTGACCGCCCACCAGAGCAGCGTGGGCAGCGTGCTGCAGGCCGGCAACCAG
CTGATCACCCAGGGCACCCTGAGCGACGAGGAGGAGTTCGAGATCCAGGAGCAGATGACCCTGCTGAACGCCCGG
TGGGAGGCCCTGCGGGTGGAGAGCATGGACCGGCAGAGCCGGCTGCACGACGTGCTGATGGAGCTGCAGAAGAAG
CAGCTGCAGCAGCTGAGCGCCTGGCTGACCCTGACCGAGGAGCGGATCCAGAAGATGGAGACCTGCCCCCTGGAC
GACGACGTGAAGAGCCTGCAGAAGCTGCTGGAGGAGCACAAGAGCCTGCAGAGCGACCTGGAGGCCGAGCAGGTG
AAGGTGAACAGCCTGACCCACATGGTGGTGATCGTGGACGAGAACAGCGGCGAGAGCGCCACCGCCATCCTGGAG
GACCAGCTGCAGAAGCTGGGCGAGCGGTGGACCGCCGTGTGCCGGTGGACCGAGGAGCGGTGGAACCGGCTGCAG
GAGATCAACATCCTGTGGCAGGAGCTGCTGGAGGAGCAGTGCCTGCTGAAGGCCTGGCTGACCGAGAAGGAGGAG
GCCCTGAACAAGGTGCAGACCAGCAACTTCAAGGACCAGAAGGAGCTGAGCGTGAGCGTGCGGCGGCTGGCCATC
CTGAAGGAGGACATGGAGATGAAGCGGCAGACCCTGGACCAGCTGAGCGAGATCGGCCAGGACGTGGGCCAGCTG
CTGGACAACAGCAAGGCCAGCAAGAAGATCAACAGCGACAGCGAGGAGCTGACCCAGCGGTGGGACAGCCTGGTG
CAGCGGCTGGAGGACAGCAGCAACCAGGTGACCCAGGCCGCCGCTAGCGCCAGCAGCGACCAGTGGAAGCGGCTG
CACCTGAGCCTGCAGGAGCTGCTGGTGTGGCTGCAGCTGAAGGACGACGAGCTGAGCCGGCAGGCCCCCATCGGC
GGCGACTTCCCCGCCGTGCAGAAGCAGAACGACGTGCACCGGGCCTTCAAGCGGGAGCTGAAGACCAAGGAGCCC
GTGATCATGAGCACCCTGGAGACCGTGCGGATCTTCCTGACCGAGCAGCCCCTGGAGGGCCTGGAGAAGCTGTAC
CAGGAGCCCCGGGAGCTGCCCCCCGAGGAGCGGGCCCAGAACGTGACCCGGCTGCTGCGGAAGCAGGCCGAGGAG
GTGAACACCGAGTGGGAGAAGCTGAACCTGCACAGCGCCGACCAGAGAAAAATAGATGAGACCCTTGAAAGACTC
CGGGAACTTCAAGAGGCCACGGATGAGCTGGACCTCAAGCTGCGCCAAGCTGAGGTGATCAAGGGATCCTGGCAG
CCCGTGGGCGATCTCCTCATTGACTCTCTCCAAGATCACCTCGAGAAAGTCAAGGCACTTCGAGGAGAAATTGCG
CCTCTGAAAGAGAACGTGAGCCACGTCAATGACCTTGCTCGCCAGCTTACCACTTTGGGCATTCAGCTCTCACCG
TATAACCTCAGCACTCTGGAAGACCTGAACACCAGATGGAAGCTTCTGCAGGTGGCCGTCGAGGACCGAGTCAGG
CAGCTGCATGAAGCCCACAGGGACTTTGGTCCAGCATCTCAGCACTTTCTTTCCACGTCTGTCCAGGGTCCCTGG
GAGAGAGCCATCTCGCCAAACAAAGTGCCCTACTATATCAACCACGAGACTCAAACAACTTGCTGGGACCATCCC
AAAATGACAGAGCTCTACCAGTCTTTAGCTGACCTGAATAATGTCAGATTCTCAGCTTATAGGACTGCCATGAAA
CTCCGAAGACTGCAGAAGGCCCTTTGCTTGGATCTCTTGAGCCTGTCAGCTGCATGTGATGCCTTGGACCAGCAC
AACCTCAAGCAAAATGACCAGCCCATGGATATCCTGCAGATTATTAATTGTTTGACCACTATTTATGACCGCCTG
GAGCAAGAGCACAACAATTTGGTCAACGTCCCTCTCTGCGTGGATATGTGTCTGAACTGGCTGCTGAATGTTTAT
GATACGGGACGAACAGGGAGGATCCGTGTCCTGTCTTTTAAAACTGGCATCATTTCCCTGTGTAAAGCACATTTG
GAAGACAAGTACAGATACCTTTTCAAGCAAGTGGCAAGTTCAACAGGATTTTGTGACCAGCGCAGGCTGGGCCTC
CTTCTGCATGATTCTATCCAAATTCCAAGACAGTTGGGTGAAGTTGCATCCTTTGGGGGCAGTAACATTGAGCCA
AGTGTCCGGAGCTGCTTCCAATTTGCTAATAATAAGCCAGAGATCGAAGCGGCCCTCTTCCTAGACTGGATGAGA
CTGGAACCCCAGTCCATGGTGTGGCTGCCCGTCCTGCACAGAGTGGCTGCTGCAGAAACTGCCAAGCATCAGGCC
AAATGTAACATCTGCAAAGAGTGTCCAATCATTGGATTCAGGTACAGGAGTCTAAAGCACTTTAATTATGACATC
TGCCAAAGCTGCTTTTTTTCTGGTCGAGTTGCAAAAGGCCATAAAATGCACTATCCCATGGTGGAATATTGCACT
CCGACTACATCAGGAGAAGATGTTCGAGACTTTGCCAAGGTACTAAAAAACAAATTTCGAACCAAAAGGTATTTT
GCGAAGCATCCCCGAATGGGCTACCTGCCAGTGCAGACTGTCTTAGAGGGGGACAACATGGAAACTCCCGTTACT
CTGATCAACTTCTGGCCAGTAGATTCTGCGCCTGCCTCGTCCCCTCAGCTTTCACACGATGATACTCATTCACGC
ATTGAACATTATGCTAGCAGGCTAGCAGAAATGGAAAACAGCAATGGATCTTATCTAAATGATAGCATCTCTCCT
AATGAGAGCATAGATGATGAACATTTGTTAATCCAGCATTACTGCCAAAGTTTGAACCAGGACTCCCCCCTGAGC
CAGCCTCGTAGTCCTGCCCAGATCTTGATTTCCTTAGAGAGTGAGGAAAGAGGGGAGCTAGAGAGAATCCTAGCA
GATCTTGAGGAAGAAAACAGGAATCTGCAAGCAGAATATGACCGTCTAAAGCAGCAGCACGAACATAAAGGCCTG
TCCCCACTGCCGTCCCCTCCTGAAATGATGCCCACCTCTCCCCAGAGTCCC

A nucleotide sequence encoding a micro-dystrophin protein optimized for skeletal muscle is further understood to include nucleotide sequences that are variants of any one of SEQ ID Nos. 22-39, 102-119, and 133-148. Variant nucleotide sequences include sequences that differ by one or more (e.g., 1, 2, 3, 4, 5, 6, 7, 8, 9, 10, 11, 12, 13, 14, 15, 16, 17, 18, 19, 20, or more) nucleotide substitutions, additions or deletions, such as allelic variants, and will, therefore, include coding sequences that differ from the nucleotide sequence of the coding sequence designated in any one of SEQ ID Nos. 22-39, 102-119, and 133-148.

In some embodiments, a micro-dystrophin protein optimized for skeletal muscle is encoded by a nucleic acid comprising a nucleotide sequence that is at least at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 22-39, 102-119, and 133-148.

In some embodiments, a micro-dystrophin protein optimized for skeletal muscle is encoded by a nucleic acid consisting essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 22-39, 102-119, and 133-148.

In some embodiments, a micro-dystrophin protein optimized for skeletal muscle is encoded by a nucleic acid consisting of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID Nos. 22-39, 102-119, and 133-148.

In some embodiments, it is desirable to overexpress micro-dystrophin proteins in skeletal muscle. Thus, in some embodiments, a nucleotide sequence encoding a micro-dystrophin protein optimized for skeletal muscle may be codon optimized for higher expression. In some embodiments, one or more codons (e.g., 1, 2, 3, 4, 5, 6, 7, 8, 9, 10, 11, 12, 13, 14, 15, 16, 17, 18, 19, 20, or more) are substituted.

In some embodiments, a micro-dystrophin protein as described herein further comprises one or more utrophin protein domains.

Non-limiting examples of amino acid sequences of micro-dystrophins for use in both heart and skeletal muscle are provided below. These constructs include the known syntrophin binding sites in dystrophin within repeat 17, repeat 22, and within the C-terminus prior to the coiled coil region.

Nterm-R2_R17_H3_R22_H4-trCterm + SynBS
(SEQ ID NO: 156)
MLWWEEVEDCYEREDVQKKTFTKWVNAQFSKFGKQHIENLFSDLQ
DGRRLLDLLEGLTGQKLPKEKGSTRVHALNNVNKALRVLQNNNVD
LVNIGSTDIVDGNHKLTLGLIWNIILHWQVKNVMKNIMAGLQQTN
SEKILLSWVRQSTRNYPQVNVINFTTSWSDGLALNALIHSHRPDL
FDWNSVVCQQSATQRLEHAFNIARYQLGIEKLLDPEDVDTTYPDK
KSILMYITSLFQVLPQQVSIEAIQEVEMLPRPPKVTKEEHFQLHH
QMHYSQQITVSLAQGYERTSSPKPRFKSYAYTQAAYVTTSDPTRS
PFPSQHLEAPEDKSFGSSLMESEVNLDRYQTALEEVLSWLLSAED
TLQAQGEISNDVEVVKDQFHTHEGYMMDLTAHQGRVGNILQLGSK
LIGTGKLSEDEETEVQEQMNLLNSRWECLRVASMEKQSNLHRVLM
DLQNQKLKELNDWLTKTEERTRKMEEEPLGPDLEDLKRQVQQHKV
LQEDLEQEQVRVNSLTHMVVVVDESSGDHATAALEEQLKVLGDRW
ANICRWTEDRWVLLQDREDRSVEKWRRFHYDIKIFNQWLTEAEQF
LRKTQIPENWEHAKYKWYLKELQDGIGQRQTVVRTLNATGEEIIQ
QSSKTDASILQEKLGSLNLRWQEVCKQLSDRKKRLEEQKQPDLAP
GLTTIGASPTQTVTLVTQPVVTKETAISKLEMPSSLMLEVPEETH
RLLQQFPLDLEKFLAWLTEAETTANVLQDATRKERLLEDSKGVKE
LMKQWQDLQGEIEAHTDVYHNLDENSQKILRSLEGSDDAVLLQRR
LDNMNEKWSELRKKSLNIRSHLEASSDQWKRLGPASQHELSTSVQ
GPWERAISPNKVPYYINHETQTTCWDHPKMTELYQSLADLNNVRF
SAYRTAMKLRRLQKALCLDLLSLSAACDALDQHNLKQNDQPMDIL
QIINCLTTIYDRLEQEHNNLVNVPLCVDMCLNWLLNVYDTGRIGR
IRVLSFKTGIISLCKAHLEDKYRYLFKQVASSTGFCDQRRLGLLL
HDSIQIPRQLGEVASFGGSNIEPSVRSCFQFANNKPEIEAALFLD
WMRLEPQSMVWLPVLHRVAAAETAKHQAKCNICKECPIIGFRYRS
LKHFNYDICQSCFFSGRVAKGHKMHYPMVEYCTPTTSGEDVRDFA
KVLKNKFRTKRYFAKHPRMGYLPVQTVLEGDNMETPVTLINFWPV
DSAPASSPQLSHDDTHSRIEHYASRLAEMENSNGSYLNDSISPNE
SIDDEHLLIQHYCQSLNQDSPLSQPRS
Nterm-R1_R17_H3_R21-R22_H4-trCterm + SynBS
(SEQ ID NO: 157)
MLWWEEVEDCYEREDVQKKTFTKWVNAQFSKFGKQHIENLFSDLQ
DGRRLLDLLEGLTGQKLPKEKGSTRVHALNNVNKALRVLQNNNVD
LVNIGSTDIVDGNHKLTLGLIWNIILHWQVKNVMKNIMAGLQQTN
SEKILLSWVRQSTRNYPQVNVINFTTSWSDGLALNALIHSHRPDL
FDWNSVVCQQSATQRLEHAFNIARYQLGIEKLLDPEDVDTTYPDK
KSILMYITSLFQVLPQQVSIEAIQEVEMLPRPPKVTKEEHFQLHH
QMHYSQQITVSLAQGYERTSSPKPRFKSYAYTQAAYVTTSDPTRS
PFPSQHLEAPEDKSFGSSLMESEVNLDRYQTALEEVLSWLLSAED
TLQAQGEISNDVEVVKDQFHTHEGYMMDLTAHQGRVGNILQLGSK
LIGTGKLSEDEETEVQEQMNLLNSRWECLRVASMEKQSNLHRVLM
DRFDRSVEKWRRFHYDIKIFNQWLTEAEQFLRKTQIPENWEHAKY
KWYLKELQDGIGQRQTVVRTLNATGEEIIQQSSKTDASILQEKLG
SLNLRWQEVCKQLSDRKKRLEEQKQPDLAPGLTTIGASPTQTVTL
VTQPVVTKETAISKLEMPSSLMLEVPKDSTQWLEAKEEAEQVLGQ
ARAKLESWKEGPYTVDAIQKKITETKQLAKDLRQWQTNVDVANDL
ALKLLRDYSADDTRKVHMITENINASWRSIHKRVSEREAALEETH
RLLQQFPLDLEKFLAWLTEAETTANVLQDATRKERLLEDSKGVKE
LMKQWQDLQGEIEAHTDVYHNLDENSQKILRSLEGSDDAVLLQRR
LDNMNFKWSELRKKSLNIRSHLEASSDQWKRLGPASQHELSTSVQ
GPWERAISPNKVPYYINHETQTTCWDHPKMTELYQSLADLNNVRE
SAYRTAMKLRRLQKALCLDLLSLSAACDALDQHNLKQNDQPMDIL
QIINCLTTIYDRLEQEHNNLVNVPLCVDMCLNWLLNVYDTGRIGR
IRVLSFKTGIISLCKAHLEDKYRYLFKQVASSTGFCDQRRLGLLL
HDSIQIPRQLGEVASFGGSNIEPSVRSCFQFANNKPEIEAALFLD
WMRLEPQSMVWLPVLHRVAAAETAKHQAKCNICKECPIIGFRYRS
LKHFNYDICQSCFFSGRVAKGHKMHYPMVEYCTPTTSGEDVRDFA
KVLKNKFRTKRYFAKHPRMGYLPVQTVLEGDNMETPVTLINFWPV
DSAPASSPQLSHDDTHSRIEHYASRLAEMENSNGSYLNDSISPNE
SIDDEHLLIQHYCQSLNQDSPLSQPRS
Nterm-R2_R17_H3_R22_H4-trCterm +
SynBS + coiled coil
(SEQ ID NO: 158)
MLWWEEVEDCYEREDVQKKTFTKWVNAQFSKFGKQHIENLFSDLQ
DGRRLLDLLEGLTGQKLPKEKGSTRVHALNNVNKALRVLQNNNVD
LVNIGSTDIVDGNHKLTLGLIWNIILHWQVKNVMKNIMAGLQQTN
SEKILLSWVRQSTRNYPQVNVINFTTSWSDGLALNALIHSHRPDL
FDWNSVVCQQSATQRLEHAFNIARYQLGIEKLLDPEDVDTTYPDK
KSILMYITSLFQVLPQQVSIEAIQEVEMLPRPPKVTKEEHFQLHH
QMHYSQQITVSLAQGYERTSSPKPRFKSYAYTQAAYVTTSDPTRS
PFPSQHLEAPEDKSFGSSLMESEVNLDRYQTALEEVLSWLLSAED
TLQAQGEISNDVEVVKDQFHTHEGYMMDLTAHQGRVGNILQLGSK
LIGTGKLSEDEETEVQEQMNLLNSRWECLRVASMEKQSNLHRVLM
DLQNQKLKELNDWLTKTEERTRKMEEEPLGPDLEDLKRQVQQHKV
LQEDLEQEQVRVNSLTHMVVVVDESSGDHATAALEEQLKVLGDRW
ANICRWTEDRWVLLQDREDRSVEKWRRFHYDIKIFNQWLTEAEQF
LRKTQIPENWEHAKYKWYLKELQDGIGQRQTVVRILNATGEEIIQ
QSSKTDASILQEKLGSLNLRWQEVCKQLSDRKKRLEEQKQPDLAP
GLTTIGASPTQTVTLVTQPVVTKETAISKLEMPSSLMLEVPEETH
RLLQQFPLDLEKFLAWLTEAETTANVLQDATRKERLLEDSKGVKE
LMKQWQDLQGEIEAHTDVYHNLDENSQKILRSLEGSDDAVLLQRR
LDNMNFKWSELRKKSLNIRSHLEASSDQWKRLGPASQHELSTSVQ
GPWERAISPNKVPYYINHETQTTCWDHPKMTELYQSLADLNNVRF
SAYRTAMKLRRLQKALCLDLLSLSAACDALDQHNLKQNDQPMDIL
QIINCLTTIYDRLEQEHNNLVNVPLCVDMCLNWLLNVYDTGRIGR
IRVLSFKTGIISLCKAHLEDKYRYLFKQVASSTGFCDQRRLGLLL
HDSIQIPRQLGEVASFGGSNIEPSVRSCFQFANNKPEIEAALFLD
WMRLEPQSMVWLPVLHRVAAAETAKHQAKCNICKECPIIGFRYRS
LKHFNYDICQSCFFSGRVAKGHKMHYPMVEYCTPTTSGEDVRDFA
KVLKNKFRTKRYFAKHPRMGYLPVQTVLEGDNMETPVTLINFWPV
DSAPASSPQLSHDDTHSRIEHYASRLAEMENSNGSYLNDSISPNE
SIDDEHLLIQHYCQSLNQDSPLSQPRSPAQILISLESEERGELER
ILADLEEENRNLQAEYDRLKQQHEHKGLSPLPSPPEMMPTSPQSP
Nterm-R1_R17_H3_R21-R22_H4-trCterm +
SynBS + coiled coil
(SEQ ID NO: 159)
MLWWEEVEDCYEREDVQKKTFTKWVNAQFSKFGKQHIENLFSDLQ
DGRRLLDLLEGLTGQKLPKEKGSTRVHALNNVNKALRVLQNNNVD
LVNIGSTDIVDGNHKLTLGLIWNIILHWQVKNVMKNIMAGLQQTN
SEKILLSWVRQSTRNYPQVNVINFTTSWSDGLALNALIHSHRPDL
FDWNSVVCQQSATQRLEHAFNIARYQLGIEKLLDPEDVDTTYPDK
KSILMYITSLFQVLPQQVSIEAIQEVEMLPRPPKVTKEEHFQLHH
QMHYSQQITVSLAQGYERTSSPKPRFKSYAYTQAAYVTTSDPTRS
PFPSQHLEAPEDKSFGSSLMESEVNLDRYQTALEEVLSWLLSAED
TLQAQGEISNDVEVVKDQFHTHEGYMMDLTAHQGRVGNILQLGSK
LIGTGKLSEDEETEVQEQMNLLNSRWECLRVASMEKQSNLHRVLM
DRFDRSVEKWRRFHYDIKIFNQWLTEAEQFLRKTQIPENWEHAKY
KWYLKELQDGIGQRQTVVRTLNATGEEIIQQSSKTDASILQEKLG
SLNLRWQEVCKQLSDRKKRLEEQKQPDLAPGLTTIGASPTQTVTL
VTQPVVTKETAISKLEMPSSLMLEVPKDSTQWLEAKEEAEQVLGQ
ARAKLESWKEGPYTVDAIQKKITETKQLAKDLRQWQTNVDVANDL
ALKLLRDYSADDTRKVHMITENINASWRSIHKRVSEREAALEETH
RLLQQFPLDLEKFLAWLTEAETTANVLQDATRKERLLEDSKGVKE
LMKQWQDLQGEIEAHTDVYHNLDENSQKILRSLEGSDDAVLLQRR
LDNMNFKWSELRKKSLNIRSHLEASSDQWKRLGPASQHELSTSVQ
GPWERAISPNKVPYYINHETQTTCWDHPKMTELYQSLADLNNVRF
SAYRTAMKLRRLQKALCLDLLSLSAACDALDQHNLKQNDQPMDIL
QIINCLTTIYDRLEQEHNNLVNVPLCVDMCLNWLLNVYDTGRIGR
IRVLSFKTGIISLCKAHLEDKYRYLFKQVASSTGFCDQRRLGLLL
HDSIQIPRQLGEVASFGGSNIEPSVRSCFQFANNKPEIEAALFLD
WMRLEPQSMVWLPVLHRVAAAETAKHQAKCNICKECPIIGFRYRS
LKHFNYDICQSCFFSGRVAKGHKMHYPMVEYCTPTTSGEDVRDFA
KVLKNKFRTKRYFAKHPRMGYLPVQTVLEGDNMETPVTLINFWPV
DSAPASSPQLSHDDTHSRIEHYASRLAEMENSNGSYLNDSISPNE
SIDDEHLLIQHYCQSLNQDSPLSQPRSPAQILISLESEERGELER
ILADLEEENRNLQAEYDRLKQQHEHKGLSPLPSPPEMMPTSPQSP
Nterm-R2_R17_R21-R22_H4-trCterm + SynBS
(SEQ ID NO: 160)
MLWWEEVEDCYEREDVQKKTFTKWVNAQFSKFGKQHIENLFSDLQ
DGRRLLDLLEGLTGQKLPKEKGSTRVHALNNVNKALRVLQNNNVD
LVNIGSTDIVDGNHKLTLGLIWNIILHWQVKNVMKNIMAGLQQTN
SEKILLSWVRQSTRNYPQVNVINFTTSWSDGLALNALIHSHRPDL
FDWNSVVCQQSATQRLEHAFNIARYQLGIEKLLDPEDVDTTYPDK
KSILMYITSLFQVLPQQVSIEAIQEVEMLPRPPKVTKEEHFQLHH
QMHYSQQITVSLAQGYERTSSPKPRFKSYAYTQAAYVTTSDPTRS
PFPSQHLEAPEDKSFGSSLMESEVNLDRYQTALEEVLSWLLSAED
TLQAQGEISNDVEVVKDQFHTHEGYMMDLTAHQGRVGNILQLGSK
LIGTGKLSEDEETEVQEQMNLLNSRWECLRVASMEKQSNLHRVLM
DLQNQKLKELNDWLTKTEERTRKMEEEPLGPDLEDLKRQVQQHKV
LQEDLEQEQVRVNSLTHMVVVVDESSGDHATAALEEQLKVLGDRW
ANICRWTEDRWVLLQDRFDRSVEKWRRFHYDIKIFNQWLTEAEQF
LRKTQIPENWEHAKYKWYLKELQDGIGQRQTVVRTLNATGEEIIQ
QSSKTDASILQEKLGSLNLRWQEVCKQLSDRKKRLEEQKKDSTQW
LEAKEEAEQVLGQARAKLESWKEGPYTVDAIQKKITETKQLAKDL
RQWQTNVDVANDLALKLLRDYSADDTRKVHMITENINASWRSIHK
RVSEREAALEETHRLLQQFPLDLEKFLAWLTEAETTANVLQDATR
KERLLEDSKGVKELMKQWQDLQGEIEAHTDVYHNLDENSQKILRS
LEGSDDAVLLQRRLDNMNFKWSELRKKSLNIRSHLEASSDQWKRL
GPASQHELSTSVQGPWERAISPNKVPYYINHETQTTCWDHPKMTE
LYQSLADLNNVRESAYRTAMKLRRLQKALCLDLLSLSAACDALDQ
HNLKQNDQPMDILQIINCLTTIYDRLEQEHNNLVNVPLCVDMCLN
WLLNVYDTGRTGRIRVLSFKTGIISLCKAHLEDKYRYLFKQVASS
TGFCDQRRLGLLLHDSIQIPRQLGEVASEGGSNIEPSVRSCFQFA
NNKPEIEAALFLDWMRLEPQSMVWLPVLHRVAAAETAKHQAKCNI
CKECPIIGFRYRSLKHFNYDICQSCFFSGRVAKGHKMHYPMVEYC
TPTTSGEDVRDFAKVLKNKFRTKRYFAKHPRMGYLPVQTVLEGDN
METPVTLINFWPVDSAPASSPQLSHDDTHSRIEHYASRLAEMENS
NGSYLNDSISPNESIDDEHLLIQHYCQSLNQDSPLSQPRS

Non-limiting examples of nucleotide sequences encoding micro-dystrophins for use in both heart and skeletal muscle are provided below.

Nterm-R2_R17_H3_R22_H4-trCterm + SynBS
Human sequence without codon alterations:
(SEQ ID NO: 166)
ATGCTTTGGTGGGAAGAAGTAGAGGACTGTTATGAAAGAGAAGAT
GTTCAAAAGAAAACATTCACAAAATGGGTAAATGCACAATTTTCT
AAGTTTGGGAAGCAGCATATTGAGAACCTCTTCAGTGACCTACAG
GATGGGAGGCGCCTCCTAGACCTCCTCGAAGGCCTGACAGGGCAA
AAACTGCCAAAAGAAAAAGGATCCACAAGAGTTCATGCCCTGAAC
AATGTCAACAAGGCACTGCGGGTTTTGCAGAACAATAATGTTGAT
TTAGTGAATATTGGAAGTACTGACATCGTAGATGGAAATCATAAA
CTGACTCTTGGTTTGATTTGGAATATAATCCTCCACTGGCAGGTC
AAAAATGTAATGAAAAATATCATGGCTGGATTGCAACAAACCAAC
AGTGAAAAGATTCTCCTGAGCTGGGTCCGACAATCAACTCGTAAT
TATCCACAGGTTAATGTAATCAACTTCACCACCAGCTGGTCTGAT
GGCCTGGCTTTGAATGCTCTCATCCATAGTCATAGGCCAGACCTA
TTTGACTGGAATAGTGTGGTTTGCCAGCAGTCAGCCACACAACGA
CTGGAACATGCATTCAACATCGCCAGATATCAATTAGGCATAGAG
AAACTACTCGATCCTGAAGATGTTGATACCACCTATCCAGATAAG
AAGTCCATCTTAATGTACATCACATCACTCTTCCAAGTTTTGCCT
CAACAAGTGAGCATTGAAGCCATCCAGGAAGTGGAAATGTTGCCA
AGGCCACCTAAAGTGACTAAAGAAGAACATTTTCAGTTACATCAT
CAAATGCACTATTCTCAACAGATCACGGTCAGTCTAGCACAGGGA
TATGAGAGAACTTCTTCCCCTAAGCCTCGATTCAAGAGCTATGCC
TACACACAGGCTGCTTATGTCACCACCTCTGACCCTACACGGAGC
CCATTTCCTTCACAGCATTTGGAAGCTCCTGAAGACAAGTCATTT
GGCAGTTCATTGATGGAGAGTGAAGTAAACCTGGACCGTTATCAA
ACAGCTTTAGAAGAAGTATTATCGTGGCTTCTTTCTGCTGAGGAC
ACATTGCAAGCACAAGGAGAGATTTCTAATGATGTGGAAGTGGTG
AAAGACCAGTTTCATACTCATGAGGGGTACATGATGGATTTGACA
GCCCATCAGGGCCGGGTTGGTAATATTCTACAATTGGGAAGTAAG
CTGATTGGAACAGGAAAATTATCAGAAGATGAAGAAACTGAAGTA
CAAGAGCAGATGAATCTCCTAAATTCAAGATGGGAATGCCTCAGG
GTAGCTAGCATGGAAAAACAAAGCAATTTACATAGAGTTTTAATG
GATCTCCAGAATCAGAAACTGAAAGAGTTGAATGACTGGCTAACA
AAAACAGAAGAAAGAACAAGGAAAATGGAGGAAGAGCCTCTTGGA
CCTGATCTTGAAGACCTAAAACGCCAAGTACAACAACATAAGGTG
CTTCAAGAAGATCTAGAACAAGAACAAGTCAGGGTCAATTCTCTC
ACTCACATGGTGGTGGTAGTTGATGAATCTAGTGGAGATCACGCA
ACTGCTGCTTTGGAAGAACAACTTAAGGTATTGGGAGATCGATGG
GCAAACATCTGTAGATGGACAGAAGACCGCTGGGTTCTTTTACAA
GACCGATTTGACAGATCTGTTGAGAAATGGCGGCGTTTTCATTAT
GATATAAAGATATTTAATCAGTGGCTAACAGAAGCTGAACAGTTT
CTCAGAAAGACACAAATTCCTGAGAATTGGGAACATGCTAAATAC
AAATGGTATCTTAAGGAACTCCAGGATGGCATTGGGCAGCGGCAA
ACTGTTGTCAGAACATTGAATGCAACTGGGGAAGAAATAATTCAG
CAATCCTCAAAAACAGATGCCAGTATTCTACAGGAAAAATTGGGA
AGCCTGAATCTGCGGTGGCAGGAGGTCTGCAAACAGCTGTCAGAC
AGAAAAAAGAGGCTAGAAGAACAAAAGCAGCCTGACCTAGCTCCT
GGACTGACCACTATTGGAGCCTCTCCTACTCAGACTGTTACTCTG
GTGACACAACCTGTGGTTACTAAGGAAACTGCCATCTCCAAACTA
GAAATGCCATCTTCCTTGATGTTGGAGGTACCTGAAGAAACTCAT
AGATTACTGCAACAGTTCCCCCTGGACCTGGAAAAGTTTCTTGCC
TGGCTTACAGAAGCTGAAACAACTGCCAATGTCCTACAGGATGCT
ACCCGTAAGGAAAGGCTCCTAGAAGACTCCAAGGGAGTAAAAGAG
CTGATGAAACAATGGCAAGACCTCCAAGGTGAAATTGAAGCTCAC
ACAGATGTTTATCACAACCTGGATGAAAACAGCCAAAAAATCCTG
AGATCCCTGGAAGGTTCCGATGATGCAGTCCTGTTACAAAGACGT
TTGGATAACATGAACTTCAAGTGGAGTGAACTTCGGAAAAAGTCT
CTCAACATTAGGTCCCATTTGGAAGCCAGTTCTGACCAGTGGAAG
CGTCTGGGTCCAGCATCTCAGCACTTTCTTTCCACGTCTGTCCAG
GGTCCCTGGGAGAGAGCCATCTCGCCAAACAAAGTGCCCTACTAT
ATCAACCACGAGACTCAAACAACTTGCTGGGACCATCCCAAAATG
ACAGAGCTCTACCAGTCTTTAGCTGACCTGAATAATGTCAGATTC
TCAGCTTATAGGACTGCCATGAAACTCCGAAGACTGCAGAAGGCC
CTTTGCTTGGATCTCTTGAGCCTGTCAGCTGCATGTGATGCCTTG
GACCAGCACAACCTCAAGCAAAATGACCAGCCCATGGATATCCTG
CAGATTATTAATTGTTTGACCACTATTTATGACCGCCTGGAGCAA
GAGCACAACAATTTGGTCAACGTCCCTCTCTGCGTGGATATGTGT
CTGAACTGGCTGCTGAATGTTTATGATACGGGACGAACAGGGAGG
ATCCGTGTCCTGTCTTTTAAAACTGGCATCATTTCCCTGTGTAAA
GCACATTTGGAAGACAAGTACAGATACCTTTTCAAGCAAGTGGCA
AGTTCAACAGGATTTTGTGACCAGCGCAGGCTGGGCCTCCTTCTG
CATGATTCTATCCAAATTCCAAGACAGTTGGGTGAAGTTGCATCC
TTTGGGGGCAGTAACATTGAGCCAAGTGTCCGGAGCTGCTTCCAA
TTTGCTAATAATAAGCCAGAGATCGAAGCGGCCCTCTTCCTAGAC
TGGATGAGACTGGAACCCCAGTCCATGGTGTGGCTGCCCGTCCTG
CACAGAGTGGCTGCTGCAGAAACTGCCAAGCATCAGGCCAAATGT
AACATCTGCAAAGAGTGTCCAATCATTGGATTCAGGTACAGGAGT
CTAAAGCACTTTAATTATGACATCTGCCAAAGCTGCTTTTTTTCT
GGTCGAGTTGCAAAAGGCCATAAAATGCACTATCCCATGGTGGAA
TATTGCACTCCGACTACATCAGGAGAAGATGTTCGAGACTTTGCC
AAGGTACTAAAAAACAAATTTCGAACCAAAAGGTATTTTGCGAAG
CATCCCCGAATGGGCTACCTGCCAGTGCAGACTGTCTTAGAGGGG
GACAACATGGAAACTCCCGTTACTCTGATCAACTTCTGGCCAGTA
GATTCTGCGCCTGCCTCGTCCCCTCAGCTTTCACACGATGATACT
CATTCACGCATTGAACATTATGCTAGCAGGCTAGCAGAAATGGAA
AACAGCAATGGATCTTATCTAAATGATAGCATCTCTCCTAATGAG
AGCATAGATGATGAACATTTGTTAATCCAGCATTACTGCCAAAGT
TTGAACCAGGACTCCCCCCTGAGCCAGCCTCGTAGT
Human sequence with codon optimization:
(SEQ ID NO: 167)
ATGCTGTGGTGGGAGGAGGTGGAGGACTGCTACGAGCGGGAGGAC
GTGCAGAAGAAGACCTTCACCAAGTGGGTGAACGCCCAGTTCAGC
AAGTTCGGCAAGCAGCACATCGAGAACCTGTTCAGCGACCTGCAG
GACGGCCGGCGGCTGCTGGACCTGCTGGAGGGCCTGACCGGCCAG
AAGCTGCCCAAGGAGAAGGGCAGCACCCGGGTGCACGCCCTGAAC
AACGTGAACAAGGCCCTGCGGGTGCTGCAGAACAACAACGTGGAC
CTGGTGAACATCGGCAGCACCGACATCGTGGACGGCAACCACAAG
CTGACCCTGGGCCTGATCTGGAACATCATCCTGCACTGGCAGGTG
AAGAACGTGATGAAGAACATCATGGCCGGCCTGCAGCAGACCAAC
AGCGAGAAGATCCTGCTGAGCTGGGTGCGGCAGAGCACCCGGAAC
TACCCCCAGGTGAACGTGATCAACTTCACCACCAGCTGGAGCGAC
GGCCTGGCCCTGAACGCCCTGATCCACAGCCACCGGCCCGACCTG
TTCGACTGGAACAGCGTGGTGTGCCAGCAGAGCGCCACCCAGCGG
CTGGAGCACGCCTTCAACATCGCCCGGTACCAGCTGGGCATCGAG
AAGCTGCTGGACCCCGAGGACGTGGACACCACCTACCCCGACAAG
AAGAGCATCCTGATGTACATCACCAGCCTGTTCCAGGTGCTGCCC
CAGCAGGTGAGCATCGAGGCCATCCAGGAGGTGGAGATGCTGCCC
CGGCCCCCCAAGGTGACCAAGGAGGAGCACTTCCAGCTGCACCAC
CAGATGCACTACAGCCAGCAGATCACCGTGAGCCTGGCCCAGGGC
TACGAGCGGACCAGCAGCCCCAAGCCCCGGTTCAAGAGCTACGCC
TACACCCAGGCCGCCTACGTGACCACCAGCGACCCCACCCGGAGC
CCCTTCCCCAGCCAGCACCTGGAGGCCCCCGAGGACAAGAGCTTC
GGCAGCAGCCTGATGGAGAGCGAGGTGAACCTGGACCGGTACCAG
ACCGCCCTGGAGGAGGTGCTGAGCTGGCTGCTGAGCGCCGAGGAC
ACCCTGCAGGCCCAGGGCGAGATCAGCAACGACGTGGAGGTGGTG
AAGGACCAGTTCCACACCCACGAGGGCTACATGATGGACCTGACC
GCCCACCAGGGCCGGGTGGGCAACATCCTGCAGCTGGGCAGCAAG
CTGATCGGCACCGGCAAGCTGAGCGAGGACGAGGAGACCGAGGTG
CAGGAGCAGATGAACCTGCTGAACAGCCGGTGGGAGTGCCTGCGG
GTGGCCAGCATGGAGAAGCAGAGCAACCTGCACCGGGTGCTGATG
GACCTGCAGAACCAGAAGCTGAAGGAGCTGAACGACTGGCTGACC
AAGACCGAGGAGCGGACCCGGAAGATGGAGGAGGAGCCCCTGGGC
CCCGACCTGGAGGACCTGAAGCGGCAGGTGCAGCAGCACAAGGTG
CTGCAGGAGGACCTGGAGCAGGAGCAGGTGCGGGTGAACAGCCTG
ACCCACATGGTGGTGGTGGTGGACGAGAGCAGCGGCGACCACGCC
ACCGCCGCCCTGGAGGAGCAGCTGAAGGTGCTGGGCGACCGGTGG
GCCAACATCTGCCGGTGGACCGAGGACCGGTGGGTGCTGCTGCAG
GACCGGTTCGACCGGAGCGTGGAGAAGTGGCGGCGGTTCCACTAC
GACATCAAGATCTTCAACCAGTGGCTGACCGAGGCCGAGCAGTTC
CTGCGGAAGACCCAGATCCCCGAGAACTGGGAGCACGCCAAGTAC
AAGTGGTACCTGAAGGAGCTGCAGGACGGCATCGGCCAGCGGCAG
ACCGTGGTGCGGACCCTGAACGCCACCGGCGAGGAGATCATCCAG
CAGAGCAGCAAGACCGACGCCAGCATCCTGCAGGAGAAGCTGGGC
AGCCTGAACCTGCGGTGGCAGGAGGTGTGCAAGCAGCTGAGCGAC
CGGAAGAAGCGGCTGGAGGAGCAGAAGCAGCCCGACCTGGCCCCC
GGCCTGACCACCATCGGCGCCAGCCCCACCCAGACCGTGACCCTG
GTGACCCAGCCCGTGGTGACCAAGGAGACCGCCATCAGCAAGCTG
GAGATGCCCAGCAGCCTGATGCTGGAGGTGCCCGAGGAGACCCAC
CGGCTGCTGCAGCAGTTCCCCCTGGACCTGGAGAAGTTCCTGGCC
TGGCTGACCGAGGCCGAGACCACCGCCAACGTGCTGCAGGACGCC
ACCCGGAAGGAGCGGCTGCTGGAGGACAGCAAGGGCGTGAAGGAG
CTGATGAAGCAGTGGCAGGACCTGCAGGGCGAGATCGAGGCCCAC
ACCGACGTGTACCACAACCTGGACGAGAACAGCCAGAAGATCCTG
CGGAGCCTGGAGGGCAGCGACGACGCCGTGCTGCTGCAGCGGCGG
CTGGACAACATGAACTTCAAGTGGAGCGAGCTGCGGAAGAAGAGC
CTGAACATCCGGAGCCACCTGGAGGCCAGCAGCGACCAGTGGAAG
CGGCTGGGCCCCGCCAGCCAGCACTTCCTGAGCACCAGCGTGCAG
GGCCCCTGGGAGCGGGCCATCAGCCCCAACAAGGTGCCCTACTAC
ATCAACCACGAGACCCAGACCACCTGCTGGGACCACCCCAAGATG
ACCGAGCTGTACCAGAGCCTGGCCGACCTGAACAACGTGCGGTTC
AGCGCCTACCGGACCGCCATGAAGCTGCGGCGGCTGCAGAAGGCC
CTGTGCCTGGACCTGCTGAGCCTGAGCGCCGCCTGCGACGCCCTG
GACCAGCACAACCTGAAGCAGAACGACCAGCCCATGGACATCCTG
CAGATCATCAACTGCCTGACCACCATCTACGACCGGCTGGAGCAG
GAGCACAACAACCTGGTGAACGTGCCCCTGTGCGTGGACATGTGC
CTGAACTGGCTGCTGAACGTGTACGACACCGGCCGGACCGGCCGG
ATCCGGGTGCTGAGCTTCAAGACCGGCATCATCAGCCTGTGCAAG
GCCCACCTGGAGGACAAGTACCGGTACCTGTTCAAGCAGGTGGCC
AGCAGCACCGGCTTCTGCGACCAGCGGCGGCTGGGCCTGCTGCTG
CACGACAGCATCCAGATCCCCCGGCAGCTGGGCGAGGTGGCCAGC
TTCGGCGGCAGCAACATCGAGCCCAGCGTGCGGAGCTGCTTCCAG
TTCGCCAACAACAAGCCCGAGATCGAGGCCGCCCTGTTCCTGGAC
TGGATGCGGCTGGAGCCCCAGAGCATGGTGTGGCTGCCCGTGCTG
CACCGGGTGGCCGCCGCCGAGACCGCCAAGCACCAGGCCAAGTGC
AACATCTGCAAGGAGTGCCCCATCATCGGCTTCCGGTACCGGAGC
CTGAAGCACTTCAACTACGACATCTGCCAGAGCTGCTTCTTCAGC
GGCCGGGTGGCCAAGGGCCACAAGATGCACTACCCCATGGTGGAG
TACTGCACCCCCACCACCAGCGGCGAGGACGTGCGGGACTTCGCC
AAGGTGCTGAAGAACAAGTTCCGGACCAAGCGGTACTTCGCCAAG
CACCCCCGGATGGGCTACCTGCCCGTGCAGACCGTGCTGGAGGGC
GACAACATGGAGACCCCCGTGACCCTGATCAACTTCTGGCCCGTG
GACAGCGCCCCCGCCAGCAGCCCCCAGCTGAGCCACGACGACACC
CACAGCCGGATCGAGCACTACGCCAGCCGGCTGGCCGAGATGGAG
AACAGCAACGGCAGCTACCTGAACGACAGCATCAGCCCCAACGAG
AGCATCGACGACGAGCACCTGCTGATCCAGCACTACTGCCAGAGC
CTGAACCAGGACAGCCCCCTGAGCCAGCCCCGGAGC
Nterm-R1_R17_H3_R21-R22_H4-trCterm + SynBS
Human sequence without codon alterations:
(SEQ ID NO: 168)
ATGCTTTGGTGGGAAGAAGTAGAGGACTGTTATGAAAGAGAAGAT
GTTCAAAAGAAAACATTCACAAAATGGGTAAATGCACAATTTTCT
AAGTTTGGGAAGCAGCATATTGAGAACCTCTTCAGTGACCTACAG
GATGGGAGGCGCCTCCTAGACCTCCTCGAAGGCCTGACAGGGCAA
AAACTGCCAAAAGAAAAAGGATCCACAAGAGTTCATGCCCTGAAC
AATGTCAACAAGGCACTGCGGGTTTTGCAGAACAATAATGTTGAT
TTAGTGAATATTGGAAGTACTGACATCGTAGATGGAAATCATAAA
CTGACTCTTGGTTTGATTTGGAATATAATCCTCCACTGGCAGGTC
AAAAATGTAATGAAAAATATCATGGCTGGATTGCAACAAACCAAC
AGTGAAAAGATTCTCCTGAGCTGGGTCCGACAATCAACTCGTAAT
TATCCACAGGTTAATGTAATCAACTTCACCACCAGCTGGTCTGAT
GGCCTGGCTTTGAATGCTCTCATCCATAGTCATAGGCCAGACCTA
TTTGACTGGAATAGTGTGGTTTGCCAGCAGTCAGCCACACAACGA
CTGGAACATGCATTCAACATCGCCAGATATCAATTAGGCATAGAG
AAACTACTCGATCCTGAAGATGTTGATACCACCTATCCAGATAAG
AAGTCCATCTTAATGTACATCACATCACTCTTCCAAGTTTTGCCT
CAACAAGTGAGCATTGAAGCCATCCAGGAAGTGGAAATGTTGCCA
AGGCCACCTAAAGTGACTAAAGAAGAACATTTTCAGTTACATCAT
CAAATGCACTATTCTCAACAGATCACGGTCAGTCTAGCACAGGGA
TATGAGAGAACTTCTTCCCCTAAGCCTCGATTCAAGAGCTATGCC
TACACACAGGCTGCTTATGTCACCACCTCTGACCCTACACGGAGC
CCATTTCCTTCACAGCATTTGGAAGCTCCTGAAGACAAGTCATTT
GGCAGTTCATTGATGGAGAGTGAAGTAAACCTGGACCGTTATCAA
ACAGCTTTAGAAGAAGTATTATCGTGGCTTCTTTCTGCTGAGGAC
ACATTGCAAGCACAAGGAGAGATTTCTAATGATGTGGAAGTGGTG
AAAGACCAGTTTCATACTCATGAGGGGTACATGATGGATTTGACA
GCCCATCAGGGCCGGGTTGGTAATATTCTACAATTGGGAAGTAAG
CTGATTGGAACAGGAAAATTATCAGAAGATGAAGAAACTGAAGTA
CAAGAGCAGATGAATCTCCTAAATTCAAGATGGGAATGCCTCAGG
GTAGCTAGCATGGAAAAACAAAGCAATTTACATAGAGTTTTAATG
GATCGATTTGACAGATCTGTTGAGAAATGGCGGCGTTTTCATTAT
GATATAAAGATATTTAATCAGTGGCTAACAGAAGCTGAACAGTTT
CTCAGAAAGACACAAATTCCTGAGAATTGGGAACATGCTAAATAC
AAATGGTATCTTAAGGAACTCCAGGATGGCATTGGGCAGCGGCAA
ACTGTTGTCAGAACATTGAATGCAACTGGGGAAGAAATAATTCAG
CAATCCTCAAAAACAGATGCCAGTATTCTACAGGAAAAATTGGGA
AGCCTGAATCTGCGGTGGCAGGAGGTCTGCAAACAGCTGTCAGAC
AGAAAAAAGAGGCTAGAAGAACAAAAGCAGCCTGACCTAGCTCCT
GGACTGACCACTATTGGAGCCTCTCCTACTCAGACTGTTACTCTG
GTGACACAACCTGTGGTTACTAAGGAAACTGCCATCTCCAAACTA
GAAATGCCATCTTCCTTGATGTTGGAGGTACCTAAGGATTCAACA
CAATGGCTGGAAGCTAAGGAAGAAGCTGAGCAGGTCTTAGGACAG
GCCAGAGCCAAGCTTGAGTCATGGAAGGAGGGTCCCTATACAGTA
GATGCAATCCAAAAGAAAATCACAGAAACCAAGCAGTTGGCCAAA
GACCTCCGCCAGTGGCAGACAAATGTAGATGTGGCAAATGACTTG
GCCCTGAAACTTCTCCGGGATTATTCTGCAGATGATACCAGAAAA
GTCCACATGATAACAGAGAATATCAATGCCTCTTGGAGAAGCATT
CATAAAAGGGTGAGTGAGCGAGAGGCTGCTTTGGAAGAAACTCAT
AGATTACTGCAACAGTTCCCCCTGGACCTGGAAAAGTTTCTTGCC
TGGCTTACAGAAGCTGAAACAACTGCCAATGTCCTACAGGATGCT
ACCCGTAAGGAAAGGCTCCTAGAAGACTCCAAGGGAGTAAAAGAG
CTGATGAAACAATGGCAAGACCTCCAAGGTGAAATTGAAGCTCAC
ACAGATGTTTATCACAACCTGGATGAAAACAGCCAAAAAATCCTG
AGATCCCTGGAAGGTTCCGATGATGCAGTCCTGTTACAAAGACGT
TTGGATAACATGAACTTCAAGTGGAGTGAACTTCGGAAAAAGTCT
CTCAACATTAGGTCCCATTTGGAAGCCAGTTCTGACCAGTGGAAG
CGTCTGGGTCCAGCATCTCAGCACTTTCTTTCCACGTCTGTCCAG
GGTCCCTGGGAGAGAGCCATCTCGCCAAACAAAGTGCCCTACTAT
ATCAACCACGAGACTCAAACAACTTGCTGGGACCATCCCAAAATG
ACAGAGCTCTACCAGTCTTTAGCTGACCTGAATAATGTCAGATTC
TCAGCTTATAGGACTGCCATGAAACTCCGAAGACTGCAGAAGGCC
CTTTGCTTGGATCTCTTGAGCCTGTCAGCTGCATGTGATGCCTTG
GACCAGCACAACCTCAAGCAAAATGACCAGCCCATGGATATCCTG
CAGATTATTAATTGTTTGACCACTATTTATGACCGCCTGGAGCAA
GAGCACAACAATTTGGTCAACGTCCCTCTCTGCGTGGATATGTGT
CTGAACTGGCTGCTGAATGTTTATGATACGGGACGAACAGGGAGG
ATCCGTGTCCTGTCTTTTAAAACTGGCATCATTTCCCTGTGTAAA
GCACATTTGGAAGACAAGTACAGATACCTTTTCAAGCAAGTGGCA
AGTTCAACAGGATTTTGTGACCAGCGCAGGCTGGGCCTCCTTCTG
CATGATTCTATCCAAATTCCAAGACAGTTGGGTGAAGTTGCATCC
TTTGGGGGCAGTAACATTGAGCCAAGTGTCCGGAGCTGCTTCCAA
TTTGCTAATAATAAGCCAGAGATCGAAGCGGCCCTCTTCCTAGAC
TGGATGAGACTGGAACCCCAGTCCATGGTGTGGCTGCCCGTCCTG
CACAGAGTGGCTGCTGCAGAAACTGCCAAGCATCAGGCCAAATGT
AACATCTGCAAAGAGTGTCCAATCATTGGATTCAGGTACAGGAGT
CTAAAGCACTTTAATTATGACATCTGCCAAAGCTGCTTTTTTTCT
GGTCGAGTTGCAAAAGGCCATAAAATGCACTATCCCATGGTGGAA
TATTGCACTCCGACTACATCAGGAGAAGATGTTCGAGACTTTGCC
AAGGTACTAAAAAACAAATTTCGAACCAAAAGGTATTTTGCGAAG
CATCCCCGAATGGGCTACCTGCCAGTGCAGACTGTCTTAGAGGGG
GACAACATGGAAACTCCCGTTACTCTGATCAACTTCTGGCCAGTA
GATTCTGCGCCTGCCTCGTCCCCTCAGCTTTCACACGATGATACT
CATTCACGCATTGAACATTATGCTAGCAGGCTAGCAGAAATGGAA
AACAGCAATGGATCTTATCTAAATGATAGCATCTCTCCTAATGAG
AGCATAGATGATGAACATTTGTTAATCCAGCATTACTGCCAAAGT
TTGAACCAGGACTCCCCCCTGAGCCAGCCTCGTAGT
Human sequence with codon optimization:
(SEQ ID NO: 169)
ATGCTGTGGTGGGAGGAGGTGGAGGACTGCTACGAGCGGGAGGAC
GTGCAGAAGAAGACCTTCACCAAGTGGGTGAACGCCCAGTTCAGC
AAGTTCGGCAAGCAGCACATCGAGAACCTGTTCAGCGACCTGCAG
GACGGCCGGCGGCTGCTGGACCTGCTGGAGGGCCTGACCGGCCAG
AAGCTGCCCAAGGAGAAGGGCAGCACCCGGGTGCACGCCCTGAAC
AACGTGAACAAGGCCCTGCGGGTGCTGCAGAACAACAACGTGGAC
CTGGTGAACATCGGCAGCACCGACATCGTGGACGGCAACCACAAG
CTGACCCTGGGCCTGATCTGGAACATCATCCTGCACTGGCAGGTG
AAGAACGTGATGAAGAACATCATGGCCGGCCTGCAGCAGACCAAC
AGCGAGAAGATCCTGCTGAGCTGGGTGCGGCAGAGCACCCGGAAC
TACCCCCAGGTGAACGTGATCAACTTCACCACCAGCTGGAGCGAC
GGCCTGGCCCTGAACGCCCTGATCCACAGCCACCGGCCCGACCTG
TTCGACTGGAACAGCGTGGTGTGCCAGCAGAGCGCCACCCAGCGG
CTGGAGCACGCCTTCAACATCGCCCGGTACCAGCTGGGCATCGAG
AAGCTGCTGGACCCCGAGGACGTGGACACCACCTACCCCGACAAG
AAGAGCATCCTGATGTACATCACCAGCCTGTTCCAGGTGCTGCCC
CAGCAGGTGAGCATCGAGGCCATCCAGGAGGTGGAGATGCTGCCC
CGGCCCCCCAAGGTGACCAAGGAGGAGCACTTCCAGCTGCACCAC
CAGATGCACTACAGCCAGCAGATCACCGTGAGCCTGGCCCAGGGC
TACGAGCGGACCAGCAGCCCCAAGCCCCGGTTCAAGAGCTACGCC
TACACCCAGGCCGCCTACGTGACCACCAGCGACCCCACCCGGAGC
CCCTTCCCCAGCCAGCACCTGGAGGCCCCCGAGGACAAGAGCTTC
GGCAGCAGCCTGATGGAGAGCGAGGTGAACCTGGACCGGTACCAG
ACCGCCCTGGAGGAGGTGCTGAGCTGGCTGCTGAGCGCCGAGGAC
ACCCTGCAGGCCCAGGGCGAGATCAGCAACGACGTGGAGGTGGTG
AAGGACCAGTTCCACACCCACGAGGGCTACATGATGGACCTGACC
GCCCACCAGGGCCGGGTGGGCAACATCCTGCAGCTGGGCAGCAAG
CTGATCGGCACCGGCAAGCTGAGCGAGGACGAGGAGACCGAGGTG
CAGGAGCAGATGAACCTGCTGAACAGCCGGTGGGAGTGCCTGCGG
GTGGCCAGCATGGAGAAGCAGAGCAACCTGCACCGGGTGCTGATG
GACCGGTTCGACCGGAGCGTGGAGAAGTGGCGGCGGTTCCACTAC
GACATCAAGATCTTCAACCAGTGGCTGACCGAGGCCGAGCAGTTC
CTGCGGAAGACCCAGATCCCCGAGAACTGGGAGCACGCCAAGTAC
AAGTGGTACCTGAAGGAGCTGCAGGACGGCATCGGCCAGCGGCAG
ACCGTGGTGCGGACCCTGAACGCCACCGGCGAGGAGATCATCCAG
CAGAGCAGCAAGACCGACGCCAGCATCCTGCAGGAGAAGCTGGGC
AGCCTGAACCTGCGGTGGCAGGAGGTGTGCAAGCAGCTGAGCGAC
CGGAAGAAGCGGCTGGAGGAGCAGAAGCAGCCCGACCTGGCCCCC
GGCCTGACCACCATCGGCGCCAGCCCCACCCAGACCGTGACCCTG
GTGACCCAGCCCGTGGTGACCAAGGAGACCGCCATCAGCAAGCTG
GAGATGCCCAGCAGCCTGATGCTGGAGGTGCCCAAGGACAGCACC
CAGTGGCTGGAGGCCAAGGAGGAGGCCGAGCAGGTGCTGGGCCAG
GCCCGGGCCAAGCTGGAGAGCTGGAAGGAGGGCCCCTACACCGTG
GACGCCATCCAGAAGAAGATCACCGAGACCAAGCAGCTGGCCAAG
GACCTGCGGCAGTGGCAGACCAACGTGGACGTGGCCAACGACCTG
GCCCTGAAGCTGCTGCGGGACTACAGCGCCGACGACACCCGGAAG
GTGCACATGATCACCGAGAACATCAACGCCAGCTGGCGGAGCATC
CACAAGCGGGTGAGCGAGCGGGAGGCCGCCCTGGAGGAGACCCAC
CGGCTGCTGCAGCAGTTCCCCCTGGACCTGGAGAAGTTCCTGGCC
TGGCTGACCGAGGCCGAGACCACCGCCAACGTGCTGCAGGACGCC
ACCCGGAAGGAGCGGCTGCTGGAGGACAGCAAGGGCGTGAAGGAG
CTGATGAAGCAGTGGCAGGACCTGCAGGGCGAGATCGAGGCCCAC
ACCGACGTGTACCACAACCTGGACGAGAACAGCCAGAAGATCCTG
CGGAGCCTGGAGGGCAGCGACGACGCCGTGCTGCTGCAGCGGCGG
CTGGACAACATGAACTTCAAGTGGAGCGAGCTGCGGAAGAAGAGC
CTGAACATCCGGAGCCACCTGGAGGCCAGCAGCGACCAGTGGAAG
CGGCTGGGCCCCGCCAGCCAGCACTTCCTGAGCACCAGCGTGCAG
GGCCCCTGGGAGCGGGCCATCAGCCCCAACAAGGTGCCCTACTAC
ATCAACCACGAGACCCAGACCACCTGCTGGGACCACCCCAAGATG
ACCGAGCTGTACCAGAGCCTGGCCGACCTGAACAACGTGCGGTTC
AGCGCCTACCGGACCGCCATGAAGCTGCGGCGGCTGCAGAAGGCC
CTGTGCCTGGACCTGCTGAGCCTGAGCGCCGCCTGCGACGCCCTG
GACCAGCACAACCTGAAGCAGAACGACCAGCCCATGGACATCCTG
CAGATCATCAACTGCCTGACCACCATCTACGACCGGCTGGAGCAG
GAGCACAACAACCTGGTGAACGTGCCCCTGTGCGTGGACATGTGC
CTGAACTGGCTGCTGAACGTGTACGACACCGGCCGGACCGGCCGG
ATCCGGGTGCTGAGCTTCAAGACCGGCATCATCAGCCTGTGCAAG
GCCCACCTGGAGGACAAGTACCGGTACCTGTTCAAGCAGGTGGCC
AGCAGCACCGGCTTCTGCGACCAGCGGCGGCTGGGCCTGCTGCTG
CACGACAGCATCCAGATCCCCCGGCAGCTGGGCGAGGTGGCCAGC
TTCGGCGGCAGCAACATCGAGCCCAGCGTGCGGAGCTGCTTCCAG
TTCGCCAACAACAAGCCCGAGATCGAGGCCGCCCTGTTCCTGGAC
TGGATGCGGCTGGAGCCCCAGAGCATGGTGTGGCTGCCCGTGCTG
CACCGGGTGGCCGCCGCCGAGACCGCCAAGCACCAGGCCAAGTGC
AACATCTGCAAGGAGTGCCCCATCATCGGCTTCCGGTACCGGAGC
CTGAAGCACTTCAACTACGACATCTGCCAGAGCTGCTTCTTCAGC
GGCCGGGTGGCCAAGGGCCACAAGATGCACTACCCCATGGTGGAG
TACTGCACCCCCACCACCAGCGGCGAGGACGTGCGGGACTTCGCC
AAGGTGCTGAAGAACAAGTTCCGGACCAAGCGGTACTTCGCCAAG
CACCCCCGGATGGGCTACCTGCCCGTGCAGACCGTGCTGGAGGGC
GACAACATGGAGACCCCCGTGACCCTGATCAACTTCTGGCCCGTG
GACAGCGCCCCCGCCAGCAGCCCCCAGCTGAGCCACGACGACACC
CACAGCCGGATCGAGCACTACGCCAGCCGGCTGGCCGAGATGGAG
AACAGCAACGGCAGCTACCTGAACGACAGCATCAGCCCCAACGAG
AGCATCGACGACGAGCACCTGCTGATCCAGCACTACTGCCAGAGC
CTGAACCAGGACAGCCCCCTGAGCCAGCCCCGGAGC
Nterm-R2_R17_H3_R22_H4-
trCterm + SynBS + coiled coil
Human sequence without codon alterations:
(SEQ ID NO: 170)
ATGCTTTGGTGGGAAGAAGTAGAGGACTGTTATGAAAGAGAAGAT
GTTCAAAAGAAAACATTCACAAAATGGGTAAATGCACAATTTTCT
AAGTTTGGGAAGCAGCATATTGAGAACCTCTTCAGTGACCTACAG
GATGGGAGGCGCCTCCTAGACCTCCTCGAAGGCCTGACAGGGCAA
AAACTGCCAAAAGAAAAAGGATCCACAAGAGTTCATGCCCTGAAC
AATGTCAACAAGGCACTGCGGGTTTTGCAGAACAATAATGTTGAT
TTAGTGAATATTGGAAGTACTGACATCGTAGATGGAAATCATAAA
CTGACTCTTGGTTTGATTTGGAATATAATCCTCCACTGGCAGGTC
AAAAATGTAATGAAAAATATCATGGCTGGATTGCAACAAACCAAC
AGTGAAAAGATTCTCCTGAGCTGGGTCCGACAATCAACTCGTAAT
TATCCACAGGTTAATGTAATCAACTTCACCACCAGCTGGTCTGAT
GGCCTGGCTTTGAATGCTCTCATCCATAGTCATAGGCCAGACCTA
TTTGACTGGAATAGTGTGGTTTGCCAGCAGTCAGCCACACAACGA
CTGGAACATGCATTCAACATCGCCAGATATCAATTAGGCATAGAG
AAACTACTCGATCCTGAAGATGTTGATACCACCTATCCAGATAAG
AAGTCCATCTTAATGTACATCACATCACTCTTCCAAGTTTTGCCT
CAACAAGTGAGCATTGAAGCCATCCAGGAAGTGGAAATGTTGCCA
AGGCCACCTAAAGTGACTAAAGAAGAACATTTTCAGTTACATCAT
CAAATGCACTATTCTCAACAGATCACGGTCAGTCTAGCACAGGGA
TATGAGAGAACTTCTTCCCCTAAGCCTCGATTCAAGAGCTATGCC
TACACACAGGCTGCTTATGTCACCACCTCTGACCCTACACGGAGC
CCATTTCCTTCACAGCATTTGGAAGCTCCTGAAGACAAGTCATTT
GGCAGTTCATTGATGGAGAGTGAAGTAAACCTGGACCGTTATCAA
ACAGCTTTAGAAGAAGTATTATCGTGGCTTCTTTCTGCTGAGGAC
ACATTGCAAGCACAAGGAGAGATTTCTAATGATGTGGAAGTGGTG
AAAGACCAGTTTCATACTCATGAGGGGTACATGATGGATTTGACA
GCCCATCAGGGCCGGGTTGGTAATATTCTACAATTGGGAAGTAAG
CTGATTGGAACAGGAAAATTATCAGAAGATGAAGAAACTGAAGTA
CAAGAGCAGATGAATCTCCTAAATTCAAGATGGGAATGCCTCAGG
GTAGCTAGCATGGAAAAACAAAGCAATTTACATAGAGTTTTAATG
GATCTCCAGAATCAGAAACTGAAAGAGTTGAATGACTGGCTAACA
AAAACAGAAGAAAGAACAAGGAAAATGGAGGAAGAGCCTCTTGGA
CCTGATCTTGAAGACCTAAAACGCCAAGTACAACAACATAAGGTG
CTTCAAGAAGATCTAGAACAAGAACAAGTCAGGGTCAATTCTCTC
ACTCACATGGTGGTGGTAGTTGATGAATCTAGTGGAGATCACGCA
ACTGCTGCTTTGGAAGAACAACTTAAGGTATTGGGAGATCGATGG
GCAAACATCTGTAGATGGACAGAAGACCGCTGGGTTCTTTTACAA
GACCGATTTGACAGATCTGTTGAGAAATGGCGGCGTTTTCATTAT
GATATAAAGATATTTAATCAGTGGCTAACAGAAGCTGAACAGTTT
CTCAGAAAGACACAAATTCCTGAGAATTGGGAACATGCTAAATAC
AAATGGTATCTTAAGGAACTCCAGGATGGCATTGGGCAGCGGCAA
ACTGTTGTCAGAACATTGAATGCAACTGGGGAAGAAATAATTCAG
CAATCCTCAAAAACAGATGCCAGTATTCTACAGGAAAAATTGGGA
AGCCTGAATCTGCGGTGGCAGGAGGTCTGCAAACAGCTGTCAGAC
AGAAAAAAGAGGCTAGAAGAACAAAAGCAGCCTGACCTAGCTCCT
GGACTGACCACTATTGGAGCCTCTCCTACTCAGACTGTTACTCTG
GTGACACAACCTGTGGTTACTAAGGAAACTGCCATCTCCAAACTA
GAAATGCCATCTTCCTTGATGTTGGAGGTACCTGAAGAAACTCAT
AGATTACTGCAACAGTTCCCCCTGGACCTGGAAAAGTTTCTTGCC
TGGCTTACAGAAGCTGAAACAACTGCCAATGTCCTACAGGATGCT
ACCCGTAAGGAAAGGCTCCTAGAAGACTCCAAGGGAGTAAAAGAG
CTGATGAAACAATGGCAAGACCTCCAAGGTGAAATTGAAGCTCAC
ACAGATGTTTATCACAACCTGGATGAAAACAGCCAAAAAATCCTG
AGATCCCTGGAAGGTTCCGATGATGCAGTCCTGTTACAAAGACGT
TTGGATAACATGAACTTCAAGTGGAGTGAACTTCGGAAAAAGTCT
CTCAACATTAGGTCCCATTTGGAAGCCAGTTCTGACCAGTGGAAG
CGTCTGGGTCCAGCATCTCAGCACTTTCTTTCCACGTCTGTCCAG
GGTCCCTGGGAGAGAGCCATCTCGCCAAACAAAGTGCCCTACTAT
ATCAACCACGAGACTCAAACAACTTGCTGGGACCATCCCAAAATG
ACAGAGCTCTACCAGTCTTTAGCTGACCTGAATAATGTCAGATTC
TCAGCTTATAGGACTGCCATGAAACTCCGAAGACTGCAGAAGGCC
CTTTGCTTGGATCTCTTGAGCCTGTCAGCTGCATGTGATGCCTTG
GACCAGCACAACCTCAAGCAAAATGACCAGCCCATGGATATCCTG
CAGATTATTAATTGTTTGACCACTATTTATGACCGCCTGGAGCAA
GAGCACAACAATTTGGTCAACGTCCCTCTCTGCGTGGATATGTGT
CTGAACTGGCTGCTGAATGTTTATGATACGGGACGAACAGGGAGG
ATCCGTGTCCTGTCTTTTAAAACTGGCATCATTTCCCTGTGTAAA
GCACATTTGGAAGACAAGTACAGATACCTTTTCAAGCAAGTGGCA
AGTTCAACAGGATTTTGTGACCAGCGCAGGCTGGGCCTCCTTCTG
CATGATTCTATCCAAATTCCAAGACAGTTGGGTGAAGTTGCATCC
TTTGGGGGCAGTAACATTGAGCCAAGTGTCCGGAGCTGCTTCCAA
TTTGCTAATAATAAGCCAGAGATCGAAGCGGCCCTCTTCCTAGAC
TGGATGAGACTGGAACCCCAGTCCATGGTGTGGCTGCCCGTCCTG
CACAGAGTGGCTGCTGCAGAAACTGCCAAGCATCAGGCCAAATGT
AACATCTGCAAAGAGTGTCCAATCATTGGATTCAGGTACAGGAGT
CTAAAGCACTTTAATTATGACATCTGCCAAAGCTGCTTTTTTTCT
GGTCGAGTTGCAAAAGGCCATAAAATGCACTATCCCATGGTGGAA
TATTGCACTCCGACTACATCAGGAGAAGATGTTCGAGACTTTGCC
AAGGTACTAAAAAACAAATTTCGAACCAAAAGGTATTTTGCGAAG
CATCCCCGAATGGGCTACCTGCCAGTGCAGACTGTCTTAGAGGGG
GACAACATGGAAACTCCCGTTACTCTGATCAACTTCTGGCCAGTA
GATTCTGCGCCTGCCTCGTCCCCTCAGCTTTCACACGATGATACT
CATTCACGCATTGAACATTATGCTAGCAGGCTAGCAGAAATGGAA
AACAGCAATGGATCTTATCTAAATGATAGCATCTCTCCTAATGAG
AGCATAGATGATGAACATTTGTTAATCCAGCATTACTGCCAAAGT
TTGAACCAGGACTCCCCCCTGAGCCAGCCTCGTAGTCCTGCCCAG
ATCTTGATTTCCTTAGAGAGTGAGGAAAGAGGGGAGCTAGAGAGA
ATCCTAGCAGATCTTGAGGAAGAAAACAGGAATCTGCAAGCAGAA
TATGACCGTCTAAAGCAGCAGCACGAACATAAAGGCCTGTCCCCA
CTGCCGTCCCCTCCTGAAATGATGCCCACCTCTCCCCAGAGTCCC
Human sequence with codon optimization:
(SEQ ID NO: 171)
ATGCTGTGGTGGGAGGAGGTGGAGGACTGCTACGAGCGGGAGGAC
GTGCAGAAGAAGACCTTCACCAAGTGGGTGAACGCCCAGTTCAGC
AAGTTCGGCAAGCAGCACATCGAGAACCTGTTCAGCGACCTGCAG
GACGGCCGGCGGCTGCTGGACCTGCTGGAGGGCCTGACCGGCCAG
AAGCTGCCCAAGGAGAAGGGCAGCACCCGGGTGCACGCCCTGAAC
AACGTGAACAAGGCCCTGCGGGTGCTGCAGAACAACAACGTGGAC
CTGGTGAACATCGGCAGCACCGACATCGTGGACGGCAACCACAAG
CTGACCCTGGGCCTGATCTGGAACATCATCCTGCACTGGCAGGTG
AAGAACGTGATGAAGAACATCATGGCCGGCCTGCAGCAGACCAAC
AGCGAGAAGATCCTGCTGAGCTGGGTGCGGCAGAGCACCCGGAAC
TACCCCCAGGTGAACGTGATCAACTTCACCACCAGCTGGAGCGAC
GGCCTGGCCCTGAACGCCCTGATCCACAGCCACCGGCCCGACCTG
TTCGACTGGAACAGCGTGGTGTGCCAGCAGAGCGCCACCCAGCGG
CTGGAGCACGCCTTCAACATCGCCCGGTACCAGCTGGGCATCGAG
AAGCTGCTGGACCCCGAGGACGTGGACACCACCTACCCCGACAAG
AAGAGCATCCTGATGTACATCACCAGCCTGTTCCAGGTGCTGCCC
CAGCAGGTGAGCATCGAGGCCATCCAGGAGGTGGAGATGCTGCCC
CGGCCCCCCAAGGTGACCAAGGAGGAGCACTTCCAGCTGCACCAC
CAGATGCACTACAGCCAGCAGATCACCGTGAGCCTGGCCCAGGGC
TACGAGCGGACCAGCAGCCCCAAGCCCCGGTTCAAGAGCTACGCC
TACACCCAGGCCGCCTACGTGACCACCAGCGACCCCACCCGGAGC
CCCTTCCCCAGCCAGCACCTGGAGGCCCCCGAGGACAAGAGCTTC
GGCAGCAGCCTGATGGAGAGCGAGGTGAACCTGGACCGGTACCAG
ACCGCCCTGGAGGAGGTGCTGAGCTGGCTGCTGAGCGCCGAGGAC
ACCCTGCAGGCCCAGGGCGAGATCAGCAACGACGTGGAGGTGGTG
AAGGACCAGTTCCACACCCACGAGGGCTACATGATGGACCTGACC
GCCCACCAGGGCCGGGTGGGCAACATCCTGCAGCTGGGCAGCAAG
CTGATCGGCACCGGCAAGCTGAGCGAGGACGAGGAGACCGAGGTG
CAGGAGCAGATGAACCTGCTGAACAGCCGGTGGGAGTGCCTGCGG
GTGGCCAGCATGGAGAAGCAGAGCAACCTGCACCGGGTGCTGATG
GACCTGCAGAACCAGAAGCTGAAGGAGCTGAACGACTGGCTGACC
AAGACCGAGGAGCGGACCCGGAAGATGGAGGAGGAGCCCCTGGGC
CCCGACCTGGAGGACCTGAAGCGGCAGGTGCAGCAGCACAAGGTG
CTGCAGGAGGACCTGGAGCAGGAGCAGGTGCGGGTGAACAGCCTG
ACCCACATGGTGGTGGTGGTGGACGAGAGCAGCGGCGACCACGCC
ACCGCCGCCCTGGAGGAGCAGCTGAAGGTGCTGGGCGACCGGTGG
GCCAACATCTGCCGGTGGACCGAGGACCGGTGGGTGCTGCTGCAG
GACCGGTTCGACCGGAGCGTGGAGAAGTGGCGGCGGTTCCACTAC
GACATCAAGATCTTCAACCAGTGGCTGACCGAGGCCGAGCAGTTC
CTGCGGAAGACCCAGATCCCCGAGAACTGGGAGCACGCCAAGTAC
AAGTGGTACCTGAAGGAGCTGCAGGACGGCATCGGCCAGCGGCAG
ACCGTGGTGCGGACCCTGAACGCCACCGGCGAGGAGATCATCCAG
CAGAGCAGCAAGACCGACGCCAGCATCCTGCAGGAGAAGCTGGGC
AGCCTGAACCTGCGGTGGCAGGAGGTGTGCAAGCAGCTGAGCGAC
CGGAAGAAGCGGCTGGAGGAGCAGAAGCAGCCCGACCTGGCCCCC
GGCCTGACCACCATCGGCGCCAGCCCCACCCAGACCGTGACCCTG
GTGACCCAGCCCGTGGTGACCAAGGAGACCGCCATCAGCAAGCTG
GAGATGCCCAGCAGCCTGATGCTGGAGGTGCCCGAGGAGACCCAC
CGGCTGCTGCAGCAGTTCCCCCTGGACCTGGAGAAGTTCCTGGCC
TGGCTGACCGAGGCCGAGACCACCGCCAACGTGCTGCAGGACGCC
ACCCGGAAGGAGCGGCTGCTGGAGGACAGCAAGGGCGTGAAGGAG
CTGATGAAGCAGTGGCAGGACCTGCAGGGCGAGATCGAGGCCCAC
ACCGACGTGTACCACAACCTGGACGAGAACAGCCAGAAGATCCTG
CGGAGCCTGGAGGGCAGCGACGACGCCGTGCTGCTGCAGCGGCGG
CTGGACAACATGAACTTCAAGTGGAGCGAGCTGCGGAAGAAGAGC
CTGAACATCCGGAGCCACCTGGAGGCCAGCAGCGACCAGTGGAAG
CGGCTGGGCCCCGCCAGCCAGCACTTCCTGAGCACCAGCGTGCAG
GGCCCCTGGGAGCGGGCCATCAGCCCCAACAAGGTGCCCTACTAC
ATCAACCACGAGACCCAGACCACCTGCTGGGACCACCCCAAGATG
ACCGAGCTGTACCAGAGCCTGGCCGACCTGAACAACGTGCGGTTC
AGCGCCTACCGGACCGCCATGAAGCTGCGGCGGCTGCAGAAGGCC
CTGTGCCTGGACCTGCTGAGCCTGAGCGCCGCCTGCGACGCCCTG
GACCAGCACAACCTGAAGCAGAACGACCAGCCCATGGACATCCTG
CAGATCATCAACTGCCTGACCACCATCTACGACCGGCTGGAGCAG
GAGCACAACAACCTGGTGAACGTGCCCCTGTGCGTGGACATGTGC
CTGAACTGGCTGCTGAACGTGTACGACACCGGCCGGACCGGCCGG
ATCCGGGTGCTGAGCTTCAAGACCGGCATCATCAGCCTGTGCAAG
GCCCACCTGGAGGACAAGTACCGGTACCTGTTCAAGCAGGTGGCC
AGCAGCACCGGCTTCTGCGACCAGCGGCGGCTGGGCCTGCTGCTG
CACGACAGCATCCAGATCCCCCGGCAGCTGGGCGAGGTGGCCAGC
TTCGGCGGCAGCAACATCGAGCCCAGCGTGCGGAGCTGCTTCCAG
TTCGCCAACAACAAGCCCGAGATCGAGGCCGCCCTGTTCCTGGAC
TGGATGCGGCTGGAGCCCCAGAGCATGGTGTGGCTGCCCGTGCTG
CACCGGGTGGCCGCCGCCGAGACCGCCAAGCACCAGGCCAAGTGC
AACATCTGCAAGGAGTGCCCCATCATCGGCTTCCGGTACCGGAGC
CTGAAGCACTTCAACTACGACATCTGCCAGAGCTGCTTCTTCAGC
GGCCGGGTGGCCAAGGGCCACAAGATGCACTACCCCATGGTGGAG
TACTGCACCCCCACCACCAGCGGCGAGGACGTGCGGGACTTCGCC
AAGGTGCTGAAGAACAAGTTCCGGACCAAGCGGTACTTCGCCAAG
CACCCCCGGATGGGCTACCTGCCCGTGCAGACCGTGCTGGAGGGC
GACAACATGGAGACCCCCGTGACCCTGATCAACTTCTGGCCCGTG
GACAGCGCCCCCGCCAGCAGCCCCCAGCTGAGCCACGACGACACC
CACAGCCGGATCGAGCACTACGCCAGCCGGCTGGCCGAGATGGAG
AACAGCAACGGCAGCTACCTGAACGACAGCATCAGCCCCAACGAG
AGCATCGACGACGAGCACCTGCTGATCCAGCACTACTGCCAGAGC
CTGAACCAGGACAGCCCCCTGAGCCAGCCCCGGAGCCCCGCCCAG
ATCCTGATCAGCCTGGAGAGCGAGGAGCGGGGCGAGCTGGAGCGG
ATCCTGGCCGACCTGGAGGAGGAGAACCGGAACCTGCAGGCCGAG
TACGACCGGCTGAAGCAGCAGCACGAGCACAAGGGCCTGAGCCCC
CTGCCCAGCCCCCCCGAGATGATGCCCACCAGCCCCCAGAGCCCC
Nterm-R1_R17_H3_R21-R22_H4-
trCterm + SynBS + coiled coil
Human sequence without codon alterations:
(SEQ ID NO: 172)
ATGCTTTGGTGGGAAGAAGTAGAGGACTGTTATGAAAGAGAAGAT
GTTCAAAAGAAAACATTCACAAAATGGGTAAATGCACAATTTTCT
AAGTTTGGGAAGCAGCATATTGAGAACCTCTTCAGTGACCTACAG
GATGGGAGGCGCCTCCTAGACCTCCTCGAAGGCCTGACAGGGCAA
AAACTGCCAAAAGAAAAAGGATCCACAAGAGTTCATGCCCTGAAC
AATGTCAACAAGGCACTGCGGGTTTTGCAGAACAATAATGTTGAT
TTAGTGAATATTGGAAGTACTGACATCGTAGATGGAAATCATAAA
CTGACTCTTGGTTTGATTTGGAATATAATCCTCCACTGGCAGGTC
AAAAATGTAATGAAAAATATCATGGCTGGATTGCAACAAACCAAC
AGTGAAAAGATTCTCCTGAGCTGGGTCCGACAATCAACTCGTAAT
TATCCACAGGTTAATGTAATCAACTTCACCACCAGCTGGTCTGAT
GGCCTGGCTTTGAATGCTCTCATCCATAGTCATAGGCCAGACCTA
TTTGACTGGAATAGTGTGGTTTGCCAGCAGTCAGCCACACAACGA
CTGGAACATGCATTCAACATCGCCAGATATCAATTAGGCATAGAG
AAACTACTCGATCCTGAAGATGTTGATACCACCTATCCAGATAAG
AAGTCCATCTTAATGTACATCACATCACTCTTCCAAGTTTTGCCT
CAACAAGTGAGCATTGAAGCCATCCAGGAAGTGGAAATGTTGCCA
AGGCCACCTAAAGTGACTAAAGAAGAACATTTTCAGTTACATCAT
CAAATGCACTATTCTCAACAGATCACGGTCAGTCTAGCACAGGGA
TATGAGAGAACTTCTTCCCCTAAGCCTCGATTCAAGAGCTATGCC
TACACACAGGCTGCTTATGTCACCACCTCTGACCCTACACGGAGC
CCATTTCCTTCACAGCATTTGGAAGCTCCTGAAGACAAGTCATTT
GGCAGTTCATTGATGGAGAGTGAAGTAAACCTGGACCGTTATCAA
ACAGCTTTAGAAGAAGTATTATCGTGGCTTCTTTCTGCTGAGGAC
ACATTGCAAGCACAAGGAGAGATTTCTAATGATGTGGAAGTGGTG
AAAGACCAGTTTCATACTCATGAGGGGTACATGATGGATTTGACA
GCCCATCAGGGCCGGGTTGGTAATATTCTACAATTGGGAAGTAAG
CTGATTGGAACAGGAAAATTATCAGAAGATGAAGAAACTGAAGTA
CAAGAGCAGATGAATCTCCTAAATTCAAGATGGGAATGCCTCAGG
GTAGCTAGCATGGAAAAACAAAGCAATTTACATAGAGTTTTAATG
GATCGATTTGACAGATCTGTTGAGAAATGGCGGCGTTTTCATTAT
GATATAAAGATATTTAATCAGTGGCTAACAGAAGCTGAACAGTTT
CTCAGAAAGACACAAATTCCTGAGAATTGGGAACATGCTAAATAC
AAATGGTATCTTAAGGAACTCCAGGATGGCATTGGGCAGCGGCAA
ACTGTTGTCAGAACATTGAATGCAACTGGGGAAGAAATAATTCAG
CAATCCTCAAAAACAGATGCCAGTATTCTACAGGAAAAATTGGGA
AGCCTGAATCTGCGGTGGCAGGAGGTCTGCAAACAGCTGTCAGAC
AGAAAAAAGAGGCTAGAAGAACAAAAGCAGCCTGACCTAGCTCCT
GGACTGACCACTATTGGAGCCTCTCCTACTCAGACTGTTACTCTG
GTGACACAACCTGTGGTTACTAAGGAAACTGCCATCTCCAAACTA
GAAATGCCATCTTCCTTGATGTTGGAGGTACCTAAGGATTCAACA
CAATGGCTGGAAGCTAAGGAAGAAGCTGAGCAGGTCTTAGGACAG
GCCAGAGCCAAGCTTGAGTCATGGAAGGAGGGTCCCTATACAGTA
GATGCAATCCAAAAGAAAATCACAGAAACCAAGCAGTTGGCCAAA
GACCTCCGCCAGTGGCAGACAAATGTAGATGTGGCAAATGACTTG
GCCCTGAAACTTCTCCGGGATTATTCTGCAGATGATACCAGAAAA
GTCCACATGATAACAGAGAATATCAATGCCTCTTGGAGAAGCATT
CATAAAAGGGTGAGTGAGCGAGAGGCTGCTTTGGAAGAAACTCAT
AGATTACTGCAACAGTTCCCCCTGGACCTGGAAAAGTTTCTTGCC
TGGCTTACAGAAGCTGAAACAACTGCCAATGTCCTACAGGATGCT
ACCCGTAAGGAAAGGCTCCTAGAAGACTCCAAGGGAGTAAAAGAG
CTGATGAAACAATGGCAAGACCTCCAAGGTGAAATTGAAGCTCAC
ACAGATGTTTATCACAACCTGGATGAAAACAGCCAAAAAATCCTG
AGATCCCTGGAAGGTTCCGATGATGCAGTCCTGTTACAAAGACGT
TTGGATAACATGAACTTCAAGTGGAGTGAACTTCGGAAAAAGTCT
CTCAACATTAGGTCCCATTTGGAAGCCAGTTCTGACCAGTGGAAG
CGTCTGGGTCCAGCATCTCAGCACTTTCTTTCCACGTCTGTCCAG
GGTCCCTGGGAGAGAGCCATCTCGCCAAACAAAGTGCCCTACTAT
ATCAACCACGAGACTCAAACAACTTGCTGGGACCATCCCAAAATG
ACAGAGCTCTACCAGTCTTTAGCTGACCTGAATAATGTCAGATTC
TCAGCTTATAGGACTGCCATGAAACTCCGAAGACTGCAGAAGGCC
CTTTGCTTGGATCTCTTGAGCCTGTCAGCTGCATGTGATGCCTTG
GACCAGCACAACCTCAAGCAAAATGACCAGCCCATGGATATCCTG
CAGATTATTAATTGTTTGACCACTATTTATGACCGCCTGGAGCAA
GAGCACAACAATTTGGTCAACGTCCCTCTCTGCGTGGATATGTGT
CTGAACTGGCTGCTGAATGTTTATGATACGGGACGAACAGGGAGG
ATCCGTGTCCTGTCTTTTAAAACTGGCATCATTTCCCTGTGTAAA
GCACATTTGGAAGACAAGTACAGATACCTTTTCAAGCAAGTGGCA
AGTTCAACAGGATTTTGTGACCAGCGCAGGCTGGGCCTCCTTCTG
CATGATTCTATCCAAATTCCAAGACAGTTGGGTGAAGTTGCATCC
TTTGGGGGCAGTAACATTGAGCCAAGTGTCCGGAGCTGCTTCCAA
TTTGCTAATAATAAGCCAGAGATCGAAGCGGCCCTCTTCCTAGAC
TGGATGAGACTGGAACCCCAGTCCATGGTGTGGCTGCCCGTCCTG
CACAGAGTGGCTGCTGCAGAAACTGCCAAGCATCAGGCCAAATGT
AACATCTGCAAAGAGTGTCCAATCATTGGATTCAGGTACAGGAGT
CTAAAGCACTTTAATTATGACATCTGCCAAAGCTGCTTTTTTTCT
GGTCGAGTTGCAAAAGGCCATAAAATGCACTATCCCATGGTGGAA
TATTGCACTCCGACTACATCAGGAGAAGATGTTCGAGACTTTGCC
AAGGTACTAAAAAACAAATTTCGAACCAAAAGGTATTTTGCGAAG
CATCCCCGAATGGGCTACCTGCCAGTGCAGACTGTCTTAGAGGGG
GACAACATGGAAACTCCCGTTACTCTGATCAACTTCTGGCCAGTA
GATTCTGCGCCTGCCTCGTCCCCTCAGCTTTCACACGATGATACT
CATTCACGCATTGAACATTATGCTAGCAGGCTAGCAGAAATGGAA
AACAGCAATGGATCTTATCTAAATGATAGCATCTCTCCTAATGAG
AGCATAGATGATGAACATTTGTTAATCCAGCATTACTGCCAAAGT
TTGAACCAGGACTCCCCCCTGAGCCAGCCTCGTAGTCCTGCCCAG
ATCTTGATTTCCTTAGAGAGTGAGGAAAGAGGGGAGCTAGAGAGA
ATCCTAGCAGATCTTGAGGAAGAAAACAGGAATCTGCAAGCAGAA
TATGACCGTCTAAAGCAGCAGCACGAACATAAAGGCCTGTCCCCA
CTGCCGTCCCCTCCTGAAATGATGCCCACCTCTCCCCAGAGTCCC
Human sequence with codon optimization:
(SEQ ID NO: 173)
ATGCTGTGGTGGGAGGAGGTGGAGGACTGCTACGAGCGGGAGGAC
GTGCAGAAGAAGACCTTCACCAAGTGGGTGAACGCCCAGTTCAGC
AAGTTCGGCAAGCAGCACATCGAGAACCTGTTCAGCGACCTGCAG
GACGGCCGGCGGCTGCTGGACCTGCTGGAGGGCCTGACCGGCCAG
AAGCTGCCCAAGGAGAAGGGCAGCACCCGGGTGCACGCCCTGAAC
AACGTGAACAAGGCCCTGCGGGTGCTGCAGAACAACAACGTGGAC
CTGGTGAACATCGGCAGCACCGACATCGTGGACGGCAACCACAAG
CTGACCCTGGGCCTGATCTGGAACATCATCCTGCACTGGCAGGTG
AAGAACGTGATGAAGAACATCATGGCCGGCCTGCAGCAGACCAAC
AGCGAGAAGATCCTGCTGAGCTGGGTGCGGCAGAGCACCCGGAAC
TACCCCCAGGTGAACGTGATCAACTTCACCACCAGCTGGAGCGAC
GGCCTGGCCCTGAACGCCCTGATCCACAGCCACCGGCCCGACCTG
TTCGACTGGAACAGCGTGGTGTGCCAGCAGAGCGCCACCCAGCGG
CTGGAGCACGCCTTCAACATCGCCCGGTACCAGCTGGGCATCGAG
AAGCTGCTGGACCCCGAGGACGTGGACACCACCTACCCCGACAAG
AAGAGCATCCTGATGTACATCACCAGCCTGTTCCAGGTGCTGCCC
CAGCAGGTGAGCATCGAGGCCATCCAGGAGGTGGAGATGCTGCCC
CGGCCCCCCAAGGTGACCAAGGAGGAGCACTTCCAGCTGCACCAC
CAGATGCACTACAGCCAGCAGATCACCGTGAGCCTGGCCCAGGGC
TACGAGCGGACCAGCAGCCCCAAGCCCCGGTTCAAGAGCTACGCC
TACACCCAGGCCGCCTACGTGACCACCAGCGACCCCACCCGGAGC
CCCTTCCCCAGCCAGCACCTGGAGGCCCCCGAGGACAAGAGCTTC
GGCAGCAGCCTGATGGAGAGCGAGGTGAACCTGGACCGGTACCAG
ACCGCCCTGGAGGAGGTGCTGAGCTGGCTGCTGAGCGCCGAGGAC
ACCCTGCAGGCCCAGGGCGAGATCAGCAACGACGTGGAGGTGGTG
AAGGACCAGTTCCACACCCACGAGGGCTACATGATGGACCTGACC
GCCCACCAGGGCCGGGTGGGCAACATCCTGCAGCTGGGCAGCAAG
CTGATCGGCACCGGCAAGCTGAGCGAGGACGAGGAGACCGAGGTG
CAGGAGCAGATGAACCTGCTGAACAGCCGGTGGGAGTGCCTGCGG
GTGGCCAGCATGGAGAAGCAGAGCAACCTGCACCGGGTGCTGATG
GACCGGTTCGACCGGAGCGTGGAGAAGTGGCGGCGGTTCCACTAC
GACATCAAGATCTTCAACCAGTGGCTGACCGAGGCCGAGCAGTTC
CTGCGGAAGACCCAGATCCCCGAGAACTGGGAGCACGCCAAGTAC
AAGTGGTACCTGAAGGAGCTGCAGGACGGCATCGGCCAGCGGCAG
ACCGTGGTGCGGACCCTGAACGCCACCGGCGAGGAGATCATCCAG
CAGAGCAGCAAGACCGACGCCAGCATCCTGCAGGAGAAGCTGGGC
AGCCTGAACCTGCGGTGGCAGGAGGTGTGCAAGCAGCTGAGCGAC
CGGAAGAAGCGGCTGGAGGAGCAGAAGCAGCCCGACCTGGCCCCC
GGCCTGACCACCATCGGCGCCAGCCCCACCCAGACCGTGACCCTG
GTGACCCAGCCCGTGGTGACCAAGGAGACCGCCATCAGCAAGCTG
GAGATGCCCAGCAGCCTGATGCTGGAGGTGCCCAAGGACAGCACC
CAGTGGCTGGAGGCCAAGGAGGAGGCCGAGCAGGTGCTGGGCCAG
GCCCGGGCCAAGCTGGAGAGCTGGAAGGAGGGCCCCTACACCGTG
GACGCCATCCAGAAGAAGATCACCGAGACCAAGCAGCTGGCCAAG
GACCTGCGGCAGTGGCAGACCAACGTGGACGTGGCCAACGACCTG
GCCCTGAAGCTGCTGCGGGACTACAGCGCCGACGACACCCGGAAG
GTGCACATGATCACCGAGAACATCAACGCCAGCTGGCGGAGCATC
CACAAGCGGGTGAGCGAGCGGGAGGCCGCCCTGGAGGAGACCCAC
CGGCTGCTGCAGCAGTTCCCCCTGGACCTGGAGAAGTTCCTGGCC
TGGCTGACCGAGGCCGAGACCACCGCCAACGTGCTGCAGGACGCC
ACCCGGAAGGAGCGGCTGCTGGAGGACAGCAAGGGCGTGAAGGAG
CTGATGAAGCAGTGGCAGGACCTGCAGGGCGAGATCGAGGCCCAC
ACCGACGTGTACCACAACCTGGACGAGAACAGCCAGAAGATCCTG
CGGAGCCTGGAGGGCAGCGACGACGCCGTGCTGCTGCAGCGGCGG
CTGGACAACATGAACTTCAAGTGGAGCGAGCTGCGGAAGAAGAGC
CTGAACATCCGGAGCCACCTGGAGGCCAGCAGCGACCAGTGGAAG
CGGCTGGGCCCCGCCAGCCAGCACTTCCTGAGCACCAGCGTGCAG
GGCCCCTGGGAGCGGGCCATCAGCCCCAACAAGGTGCCCTACTAC
ATCAACCACGAGACCCAGACCACCTGCTGGGACCACCCCAAGATG
ACCGAGCTGTACCAGAGCCTGGCCGACCTGAACAACGTGCGGTTC
AGCGCCTACCGGACCGCCATGAAGCTGCGGCGGCTGCAGAAGGCC
CTGTGCCTGGACCTGCTGAGCCTGAGCGCCGCCTGCGACGCCCTG
GACCAGCACAACCTGAAGCAGAACGACCAGCCCATGGACATCCTG
CAGATCATCAACTGCCTGACCACCATCTACGACCGGCTGGAGCAG
GAGCACAACAACCTGGTGAACGTGCCCCTGTGCGTGGACATGTGC
CTGAACTGGCTGCTGAACGTGTACGACACCGGCCGGACCGGCCGG
ATCCGGGTGCTGAGCTTCAAGACCGGCATCATCAGCCTGTGCAAG
GCCCACCTGGAGGACAAGTACCGGTACCTGTTCAAGCAGGTGGCC
AGCAGCACCGGCTTCTGCGACCAGCGGCGGCTGGGCCTGCTGCTG
CACGACAGCATCCAGATCCCCCGGCAGCTGGGCGAGGTGGCCAGC
TTCGGCGGCAGCAACATCGAGCCCAGCGTGCGGAGCTGCTTCCAG
TTCGCCAACAACAAGCCCGAGATCGAGGCCGCCCTGTTCCTGGAC
TGGATGCGGCTGGAGCCCCAGAGCATGGTGTGGCTGCCCGTGCTG
CACCGGGTGGCCGCCGCCGAGACCGCCAAGCACCAGGCCAAGTGC
AACATCTGCAAGGAGTGCCCCATCATCGGCTTCCGGTACCGGAGC
CTGAAGCACTTCAACTACGACATCTGCCAGAGCTGCTTCTTCAGC
GGCCGGGTGGCCAAGGGCCACAAGATGCACTACCCCATGGTGGAG
TACTGCACCCCCACCACCAGCGGCGAGGACGTGCGGGACTTCGCC
AAGGTGCTGAAGAACAAGTTCCGGACCAAGCGGTACTTCGCCAAG
CACCCCCGGATGGGCTACCTGCCCGTGCAGACCGTGCTGGAGGGC
GACAACATGGAGACCCCCGTGACCCTGATCAACTTCTGGCCCGTG
GACAGCGCCCCCGCCAGCAGCCCCCAGCTGAGCCACGACGACACC
CACAGCCGGATCGAGCACTACGCCAGCCGGCTGGCCGAGATGGAG
AACAGCAACGGCAGCTACCTGAACGACAGCATCAGCCCCAACGAG
AGCATCGACGACGAGCACCTGCTGATCCAGCACTACTGCCAGAGC
CTGAACCAGGACAGCCCCCTGAGCCAGCCCCGGAGCCCCGCCCAG
ATCCTGATCAGCCTGGAGAGCGAGGAGCGGGGCGAGCTGGAGCGG
ATCCTGGCCGACCTGGAGGAGGAGAACCGGAACCTGCAGGCCGAG
TACGACCGGCTGAAGCAGCAGCACGAGCACAAGGGCCTGAGCCCC
CTGCCCAGCCCCCCCGAGATGATGCCCACCAGCCCCCAGAGCCCC
Nterm-R2_R17_R21-R22_H4-trCterm + SynBS
Human sequence without codon alterations:
(SEQ ID NO: 174)
ATGCTTTGGTGGGAAGAAGTAGAGGACTGTTATGAAAGAGAAGAT
GTTCAAAAGAAAACATTCACAAAATGGGTAAATGCACAATTTTCT
AAGTTTGGGAAGCAGCATATTGAGAACCTCTTCAGTGACCTACAG
GATGGGAGGCGCCTCCTAGACCTCCTCGAAGGCCTGACAGGGCAA
AAACTGCCAAAAGAAAAAGGATCCACAAGAGTTCATGCCCTGAAC
AATGTCAACAAGGCACTGCGGGTTTTGCAGAACAATAATGTTGAT
TTAGTGAATATTGGAAGTACTGACATCGTAGATGGAAATCATAAA
CTGACTCTTGGTTTGATTTGGAATATAATCCTCCACTGGCAGGTC
AAAAATGTAATGAAAAATATCATGGCTGGATTGCAACAAACCAAC
AGTGAAAAGATTCTCCTGAGCTGGGTCCGACAATCAACTCGTAAT
TATCCACAGGTTAATGTAATCAACTTCACCACCAGCTGGTCTGAT
GGCCTGGCTTTGAATGCTCTCATCCATAGTCATAGGCCAGACCTA
TTTGACTGGAATAGTGTGGTTTGCCAGCAGTCAGCCACACAACGA
CTGGAACATGCATTCAACATCGCCAGATATCAATTAGGCATAGAG
AAACTACTCGATCCTGAAGATGTTGATACCACCTATCCAGATAAG
AAGTCCATCTTAATGTACATCACATCACTCTTCCAAGTTTTGCCT
CAACAAGTGAGCATTGAAGCCATCCAGGAAGTGGAAATGTTGCCA
AGGCCACCTAAAGTGACTAAAGAAGAACATTTTCAGTTACATCAT
CAAATGCACTATTCTCAACAGATCACGGTCAGTCTAGCACAGGGA
TATGAGAGAACTTCTTCCCCTAAGCCTCGATTCAAGAGCTATGCC
TACACACAGGCTGCTTATGTCACCACCTCTGACCCTACACGGAGC
CCATTTCCTTCACAGCATTTGGAAGCTCCTGAAGACAAGTCATTT
GGCAGTTCATTGATGGAGAGTGAAGTAAACCTGGACCGTTATCAA
ACAGCTTTAGAAGAAGTATTATCGTGGCTTCTTTCTGCTGAGGAC
ACATTGCAAGCACAAGGAGAGATTTCTAATGATGTGGAAGTGGTG
AAAGACCAGTTTCATACTCATGAGGGGTACATGATGGATTTGACA
GCCCATCAGGGCCGGGTTGGTAATATTCTACAATTGGGAAGTAAG
CTGATTGGAACAGGAAAATTATCAGAAGATGAAGAAACTGAAGTA
CAAGAGCAGATGAATCTCCTAAATTCAAGATGGGAATGCCTCAGG
GTAGCTAGCATGGAAAAACAAAGCAATTTACATAGAGTTTTAATG
GATCTCCAGAATCAGAAACTGAAAGAGTTGAATGACTGGCTAACA
AAAACAGAAGAAAGAACAAGGAAAATGGAGGAAGAGCCTCTTGGA
CCTGATCTTGAAGACCTAAAACGCCAAGTACAACAACATAAGGTG
CTTCAAGAAGATCTAGAACAAGAACAAGTCAGGGTCAATTCTCTC
ACTCACATGGTGGTGGTAGTTGATGAATCTAGTGGAGATCACGCA
ACTGCTGCTTTGGAAGAACAACTTAAGGTATTGGGAGATCGATGG
GCAAACATCTGTAGATGGACAGAAGACCGCTGGGTTCTTTTACAA
GACCGATTTGACAGATCTGTTGAGAAATGGCGGCGTTTTCATTAT
GATATAAAGATATTTAATCAGTGGCTAACAGAAGCTGAACAGTTT
CTCAGAAAGACACAAATTCCTGAGAATTGGGAACATGCTAAATAC
AAATGGTATCTTAAGGAACTCCAGGATGGCATTGGGCAGCGGCAA
ACTGTTGTCAGAACATTGAATGCAACTGGGGAAGAAATAATTCAG
CAATCCTCAAAAACAGATGCCAGTATTCTACAGGAAAAATTGGGA
AGCCTGAATCTGCGGTGGCAGGAGGTCTGCAAACAGCTGTCAGAC
AGAAAAAAGAGGCTAGAAGAACAAAAGAAGGATTCAACACAATGG
CTGGAAGCTAAGGAAGAAGCTGAGCAGGTCTTAGGACAGGCCAGA
GCCAAGCTTGAGTCATGGAAGGAGGGTCCCTATACAGTAGATGCA
ATCCAAAAGAAAATCACAGAAACCAAGCAGTTGGCCAAAGACCTC
CGCCAGTGGCAGACAAATGTAGATGTGGCAAATGACTTGGCCCTG
AAACTTCTCCGGGATTATTCTGCAGATGATACCAGAAAAGTCCAC
ATGATAACAGAGAATATCAATGCCTCTTGGAGAAGCATTCATAAA
AGGGTGAGTGAGCGAGAGGCTGCTTTGGAAGAAACTCATAGATTA
CTGCAACAGTTCCCCCTGGACCTGGAAAAGTTTCTTGCCTGGCTT
ACAGAAGCTGAAACAACTGCCAATGTCCTACAGGATGCTACCCGT
AAGGAAAGGCTCCTAGAAGACTCCAAGGGAGTAAAAGAGCTGATG
AAACAATGGCAAGACCTCCAAGGTGAAATTGAAGCTCACACAGAT
GTTTATCACAACCTGGATGAAAACAGCCAAAAAATCCTGAGATCC
CTGGAAGGTTCCGATGATGCAGTCCTGTTACAAAGACGTTTGGAT
AACATGAACTTCAAGTGGAGTGAACTTCGGAAAAAGTCTCTCAAC
ATTAGGTCCCATTTGGAAGCCAGTTCTGACCAGTGGAAGCGTCTG
GGTCCAGCATCTCAGCACTTTCTTTCCACGTCTGTCCAGGGTCCC
TGGGAGAGAGCCATCTCGCCAAACAAAGTGCCCTACTATATCAAC
CACGAGACTCAAACAACTTGCTGGGACCATCCCAAAATGACAGAG
CTCTACCAGTCTTTAGCTGACCTGAATAATGTCAGATTCTCAGCT
TATAGGACTGCCATGAAACTCCGAAGACTGCAGAAGGCCCTTTGC
TTGGATCTCTTGAGCCTGTCAGCTGCATGTGATGCCTTGGACCAG
CACAACCTCAAGCAAAATGACCAGCCCATGGATATCCTGCAGATT
ATTAATTGTTTGACCACTATTTATGACCGCCTGGAGCAAGAGCAC
AACAATTTGGTCAACGTCCCTCTCTGCGTGGATATGTGTCTGAAC
TGGCTGCTGAATGTTTATGATACGGGACGAACAGGGAGGATCCGT
GTCCTGTCTTTTAAAACTGGCATCATTTCCCTGTGTAAAGCACAT
TTGGAAGACAAGTACAGATACCTTTTCAAGCAAGTGGCAAGTTCA
ACAGGATTTTGTGACCAGCGCAGGCTGGGCCTCCTTCTGCATGAT
TCTATCCAAATTCCAAGACAGTTGGGTGAAGTTGCATCCTTTGGG
GGCAGTAACATTGAGCCAAGTGTCCGGAGCTGCTTCCAATTTGCT
AATAATAAGCCAGAGATCGAAGCGGCCCTCTTCCTAGACTGGATG
AGACTGGAACCCCAGTCCATGGTGTGGCTGCCCGTCCTGCACAGA
GTGGCTGCTGCAGAAACTGCCAAGCATCAGGCCAAATGTAACATC
TGCAAAGAGTGTCCAATCATTGGATTCAGGTACAGGAGTCTAAAG
CACTTTAATTATGACATCTGCCAAAGCTGCTTTTTTTCTGGTCGA
GTTGCAAAAGGCCATAAAATGCACTATCCCATGGTGGAATATTGC
ACTCCGACTACATCAGGAGAAGATGTTCGAGACTTTGCCAAGGTA
CTAAAAAACAAATTTCGAACCAAAAGGTATTTTGCGAAGCATCCC
CGAATGGGCTACCTGCCAGTGCAGACTGTCTTAGAGGGGGACAAC
ATGGAAACTCCCGTTACTCTGATCAACTTCTGGCCAGTAGATTCT
GCGCCTGCCTCGTCCCCTCAGCTTTCACACGATGATACTCATTCA
CGCATTGAACATTATGCTAGCAGGCTAGCAGAAATGGAAAACAGC
AATGGATCTTATCTAAATGATAGCATCTCTCCTAATGAGAGCATA
GATGATGAACATTTGTTAATCCAGCATTACTGCCAAAGTTTGAAC
CAGGACTCCCCCCTGAGCCAGCCTCGTAGT
Human sequence with codon optimization:
(SEQ ID NO: 175)
ATGCTGTGGTGGGAGGAGGTGGAGGACTGCTACGAGCGGGAGGAC
GTGCAGAAGAAGACCTTCACCAAGTGGGTGAACGCCCAGTTCAGC
AAGTTCGGCAAGCAGCACATCGAGAACCTGTTCAGCGACCTGCAG
GACGGCCGGCGGCTGCTGGACCTGCTGGAGGGCCTGACCGGCCAG
AAGCTGCCCAAGGAGAAGGGCAGCACCCGGGTGCACGCCCTGAAC
AACGTGAACAAGGCCCTGCGGGTGCTGCAGAACAACAACGTGGAC
CTGGTGAACATCGGCAGCACCGACATCGTGGACGGCAACCACAAG
CTGACCCTGGGCCTGATCTGGAACATCATCCTGCACTGGCAGGTG
AAGAACGTGATGAAGAACATCATGGCCGGCCTGCAGCAGACCAAC
AGCGAGAAGATCCTGCTGAGCTGGGTGCGGCAGAGCACCCGGAAC
TACCCCCAGGTGAACGTGATCAACTTCACCACCAGCTGGAGCGAC
GGCCTGGCCCTGAACGCCCTGATCCACAGCCACCGGCCCGACCTG
TTCGACTGGAACAGCGTGGTGTGCCAGCAGAGCGCCACCCAGCGG
CTGGAGCACGCCTTCAACATCGCCCGGTACCAGCTGGGCATCGAG
AAGCTGCTGGACCCCGAGGACGTGGACACCACCTACCCCGACAAG
AAGAGCATCCTGATGTACATCACCAGCCTGTTCCAGGTGCTGCCC
CAGCAGGTGAGCATCGAGGCCATCCAGGAGGTGGAGATGCTGCCC
CGGCCCCCCAAGGTGACCAAGGAGGAGCACTTCCAGCTGCACCAC
CAGATGCACTACAGCCAGCAGATCACCGTGAGCCTGGCCCAGGGC
TACGAGCGGACCAGCAGCCCCAAGCCCCGGTTCAAGAGCTACGCC
TACACCCAGGCCGCCTACGTGACCACCAGCGACCCCACCCGGAGC
CCCTTCCCCAGCCAGCACCTGGAGGCCCCCGAGGACAAGAGCTTC
GGCAGCAGCCTGATGGAGAGCGAGGTGAACCTGGACCGGTACCAG
ACCGCCCTGGAGGAGGTGCTGAGCTGGCTGCTGAGCGCCGAGGAC
ACCCTGCAGGCCCAGGGCGAGATCAGCAACGACGTGGAGGTGGTG
AAGGACCAGTTCCACACCCACGAGGGCTACATGATGGACCTGACC
GCCCACCAGGGCCGGGTGGGCAACATCCTGCAGCTGGGCAGCAAG
CTGATCGGCACCGGCAAGCTGAGCGAGGACGAGGAGACCGAGGTG
CAGGAGCAGATGAACCTGCTGAACAGCCGGTGGGAGTGCCTGCGG
GTGGCCAGCATGGAGAAGCAGAGCAACCTGCACCGGGTGCTGATG
GACCTGCAGAACCAGAAGCTGAAGGAGCTGAACGACTGGCTGACC
AAGACCGAGGAGCGGACCCGGAAGATGGAGGAGGAGCCCCTGGGC
CCCGACCTGGAGGACCTGAAGCGGCAGGTGCAGCAGCACAAGGTG
CTGCAGGAGGACCTGGAGCAGGAGCAGGTGCGGGTGAACAGCCTG
ACCCACATGGTGGTGGTGGTGGACGAGAGCAGCGGCGACCACGCC
ACCGCCGCCCTGGAGGAGCAGCTGAAGGTGCTGGGCGACCGGTGG
GCCAACATCTGCCGGTGGACCGAGGACCGGTGGGTGCTGCTGCAG
GACCGGTTCGACCGGAGCGTGGAGAAGTGGCGGCGGTTCCACTAC
GACATCAAGATCTTCAACCAGTGGCTGACCGAGGCCGAGCAGTTC
CTGCGGAAGACCCAGATCCCCGAGAACTGGGAGCACGCCAAGTAC
AAGTGGTACCTGAAGGAGCTGCAGGACGGCATCGGCCAGCGGCAG
ACCGTGGTGCGGACCCTGAACGCCACCGGCGAGGAGATCATCCAG
CAGAGCAGCAAGACCGACGCCAGCATCCTGCAGGAGAAGCTGGGC
AGCCTGAACCTGCGGTGGCAGGAGGTGTGCAAGCAGCTGAGCGAC
CGGAAGAAGCGGCTGGAGGAGCAGAAGAAGGACAGCACCCAGTGG
CTGGAGGCCAAGGAGGAGGCCGAGCAGGTGCTGGGCCAGGCCCGG
GCCAAGCTGGAGAGCTGGAAGGAGGGCCCCTACACCGTGGACGCC
ATCCAGAAGAAGATCACCGAGACCAAGCAGCTGGCCAAGGACCTG
CGGCAGTGGCAGACCAACGTGGACGTGGCCAACGACCTGGCCCTG
AAGCTGCTGCGGGACTACAGCGCCGACGACACCCGGAAGGTGCAC
ATGATCACCGAGAACATCAACGCCAGCTGGCGGAGCATCCACAAG
CGGGTGAGCGAGCGGGAGGCCGCCCTGGAGGAGACCCACCGGCTG
CTGCAGCAGTTCCCCCTGGACCTGGAGAAGTTCCTGGCCTGGCTG
ACCGAGGCCGAGACCACCGCCAACGTGCTGCAGGACGCCACCCGG
AAGGAGCGGCTGCTGGAGGACAGCAAGGGCGTGAAGGAGCTGATG
AAGCAGTGGCAGGACCTGCAGGGCGAGATCGAGGCCCACACCGAC
GTGTACCACAACCTGGACGAGAACAGCCAGAAGATCCTGCGGAGC
CTGGAGGGCAGCGACGACGCCGTGCTGCTGCAGCGGCGGCTGGAC
AACATGAACTTCAAGTGGAGCGAGCTGCGGAAGAAGAGCCTGAAC
ATCCGGAGCCACCTGGAGGCCAGCAGCGACCAGTGGAAGCGGCTG
GGCCCCGCCAGCCAGCACTTCCTGAGCACCAGCGTGCAGGGCCCC
TGGGAGCGGGCCATCAGCCCCAACAAGGTGCCCTACTACATCAAC
CACGAGACCCAGACCACCTGCTGGGACCACCCCAAGATGACCGAG
CTGTACCAGAGCCTGGCCGACCTGAACAACGTGCGGTTCAGCGCC
TACCGGACCGCCATGAAGCTGCGGCGGCTGCAGAAGGCCCTGTGC
CTGGACCTGCTGAGCCTGAGCGCCGCCTGCGACGCCCTGGACCAG
CACAACCTGAAGCAGAACGACCAGCCCATGGACATCCTGCAGATC
ATCAACTGCCTGACCACCATCTACGACCGGCTGGAGCAGGAGCAC
AACAACCTGGTGAACGTGCCCCTGTGCGTGGACATGTGCCTGAAC
TGGCTGCTGAACGTGTACGACACCGGCCGGACCGGCCGGATCCGG
GTGCTGAGCTTCAAGACCGGCATCATCAGCCTGTGCAAGGCCCAC
CTGGAGGACAAGTACCGGTACCTGTTCAAGCAGGTGGCCAGCAGC
ACCGGCTTCTGCGACCAGCGGCGGCTGGGCCTGCTGCTGCACGAC
AGCATCCAGATCCCCCGGCAGCTGGGCGAGGTGGCCAGCTTCGGC
GGCAGCAACATCGAGCCCAGCGTGCGGAGCTGCTTCCAGTTCGCC
AACAACAAGCCCGAGATCGAGGCCGCCCTGTTCCTGGACTGGATG
CGGCTGGAGCCCCAGAGCATGGTGTGGCTGCCCGTGCTGCACCGG
GTGGCCGCCGCCGAGACCGCCAAGCACCAGGCCAAGTGCAACATC
TGCAAGGAGTGCCCCATCATCGGCTTCCGGTACCGGAGCCTGAAG
CACTTCAACTACGACATCTGCCAGAGCTGCTTCTTCAGCGGCCGG
GTGGCCAAGGGCCACAAGATGCACTACCCCATGGTGGAGTACTGC
ACCCCCACCACCAGCGGCGAGGACGTGCGGGACTTCGCCAAGGTG
CTGAAGAACAAGTTCCGGACCAAGCGGTACTTCGCCAAGCACCCC
CGGATGGGCTACCTGCCCGTGCAGACCGTGCTGGAGGGCGACAAC
ATGGAGACCCCCGTGACCCTGATCAACTTCTGGCCCGTGGACAGC
GCCCCCGCCAGCAGCCCCCAGCTGAGCCACGACGACACCCACAGC
CGGATCGAGCACTACGCCAGCCGGCTGGCCGAGATGGAGAACAGC
AACGGCAGCTACCTGAACGACAGCATCAGCCCCAACGAGAGCATC
GACGACGAGCACCTGCTGATCCAGCACTACTGCCAGAGCCTGAAC
CAGGACAGCCCCCTGAGCCAGCCCCGGAGC

Non-limiting examples of amino acid sequences of chimeric utrophin/dystrophin micro-dystrophins for use in both heart and skeletal muscle are provided below. These constructs include the known syntrophin binding sites in dystrophin within repeat 17, repeat 22, and within the C-terminus prior to the coiled coil region.

UtroNterm-R2_DystroR17_H3_R22_H4-
trCterm + SynBS
(SEQ ID NO: 161)
MAKYGEHEASPDNGQNEFSDIIKSRSDEHNDVQKKTFTKWINARF
SKSGKPPINDMFTDLKDGRKLLDLLEGLTGTSLPKERGSTRVHAL
NNVNRVLQVLHQNNVELVNIGGTDIVDGNHKLTLGLLWSIILHWQ
VKDVMKDVMSDLQQTNSEKILLSWVRQTTRPYSQVNVLNFTTSWT
DGLAFNAVLHRHKPDLFSWDKVVKMSPIERLEHAFSKAQTYLGIE
KLLDPEDVAVQLPDKKSIIMYLTSLFEVLPQQVTIDAIREVETLP
RKYKKECEEEAINIQSTAPEEEHESPRAETPSTVTEVDMDLDSYQ
IALEEVLTWLLSAEDTFQEQDDISDDVEEVKDQFATHEAFMMELT
AHQSSVGSVLQAGNQLITQGTLSDEEEFEIQEQMILLNARWEALR
VESMDRQSRLHDVLMELQKKQLQQLSAWLTLTEERIQKMETCPLD
DDVKSLQKLLEEHKSLQSDLEAEQVKVNSLTHMVVIVDENSGESA
TAILEDQLQKLGERWTAVCRWTEERWNRLQEIAASARFDRSVEKW
RRFHYDIKIFNQWLTEAEQFLRKTQIPENWEHAKYKWYLKELQDG
IGQRQTVVRTLNATGEEIIQQSSKTDASILQEKLGSLNLRWQEVC
KQLSDRKKRLEEQKQPDLAPGLTTIGASPTQTVTLVTQPVVTKET
AISKLEMPSSLMLEVPEETHRLLQQFPLDLEKFLAWLTEAETTAN
VLQDATRKERLLEDSKGVKELMKQWQDLQGEIEAHTDVYHNLDEN
SQKILRSLEGSDDAVLLQRRLDNMNEKWSELRKKSLNIRSHLEAS
SDQWKRLGPASQHELSTSVQGPWERAISPNKVPYYINHETQTTCW
DHPKMTELYQSLADLNNVRFSAYRTAMKLRRLQKALCLDLLSLSA
ACDALDQHNLKQNDQPMDILQIINCLTTIYDRLEQEHNNLVNVPL
CVDMCLNWLLNVYDTGRIGRIRVLSFKTGIISLCKAHLEDKYRYL
FKQVASSTGFCDQRRLGLLLHDSIQIPRQLGEVASFGGSNIEPSV
RSCFQFANNKPEIEAALFLDWMRLEPQSMVWLPVLHRVAAAETAK
HQAKCNICKECPIIGFRYRSLKHFNYDICQSCFFSGRVAKGHKMH
YPMVEYCTPTTSGEDVRDFAKVLKNKFRTKRYFAKHPRMGYLPVQ
TVLEGDNMETPVTLINFWPVDSAPASSPQLSHDDTHSRIEHYASR
LAEMENSNGSYLNDSISPNESIDDEHLLIQHYCQSLNQDSPLSQP
RS
UtroNterm-R1_DystroR17_H3_R21-R22_
H4-trCterm + SynBS
(SEQ ID NO: 162)
MAKYGEHEASPDNGQNEFSDIIKSRSDEHNDVQKKTFTKWINARF
SKSGKPPINDMFTDLKDGRKLLDLLEGLTGTSLPKERGSTRVHAL
NNVNRVLQVLHQNNVELVNIGGTDIVDGNHKLTLGLLWSIILHWQ
VKDVMKDVMSDLQQTNSEKILLSWVRQTTRPYSQVNVLNFTTSWT
DGLAFNAVLHRHKPDLFSWDKVVKMSPIERLEHAFSKAQTYLGIE
KLLDPEDVAVQLPDKKSIIMYLTSLFEVLPQQVTIDAIREVETLP
RKYKKECEEEAINIQSTAPEEEHESPRAETPSTVTEVDMDLDSYQ
IALEEVLTWLLSAEDTFQEQDDISDDVEEVKDQFATHEAFMMELT
AHQSSVGSVLQAGNQLITQGTLSDEEEFEIQEQMTLLNARWEALR
VESMDRQSRLHDVLMAASARFDRSVEKWRRFHYDIKIFNQWLTEA
EQFLRKTQIPENWEHAKYKWYLKELQDGIGQRQTVVRTLNATGEE
IIQQSSKTDASILQEKLGSLNLRWQEVCKQLSDRKKRLEEQKQPD
LAPGLTTIGASPTQTVTLVTQPVVTKETAISKLEMPSSLMLEVPK
DSTQWLEAKEEAEQVLGQARAKLESWKEGPYTVDAIQKKITETKQ
LAKDLRQWQTNVDVANDLALKLLRDYSADDTRKVHMITENINASW
RSIHKRVSEREAALEETHRLLQQFPLDLEKFLAWLTEAETTANVL
QDATRKERLLEDSKGVKELMKQWQDLQGEIEAHTDVYHNLDENSQ
KILRSLEGSDDAVLLQRRLDNMNFKWSELRKKSLNIRSHLEASSD
QWKRLGPASQHELSTSVQGPWERAISPNKVPYYINHETQTTCWDH
PKMTELYQSLADLNNVRESAYRTAMKLRRLQKALCLDLLSLSAAC
DALDQHNLKQNDQPMDILQIINCLTTIYDRLEQEHNNLVNVPLCV
DMCLNWLLNVYDTGRIGRIRVLSFKTGIISLCKAHLEDKYRYLFK
QVASSTGFCDQRRLGLLLHDSIQIPRQLGEVASEGGSNIEPSVRS
CFQFANNKPEIEAALFLDWMRLEPQSMVWLPVLHRVAAAETAKHQ
AKCNICKECPIIGFRYRSLKHFNYDICQSCFFSGRVAKGHKMHYP
MVEYCTPTTSGEDVRDFAKVLKNKFRTKRYFAKHPRMGYLPVQTV
LEGDNMETPVTLINFWPVDSAPASSPQLSHDDTHSRIEHYASRLA
EMENSNGSYLNDSISPNESIDDEHLLIQHYCQSLNQDSPLSQPRS
UtroNterm-R2_DystroR17_H3_R22_H4-
trCterm + SynBS_coiled coil
(SEQ ID NO: 163)
MAKYGEHEASPDNGQNEFSDIIKSRSDEHNDVQKKTFTKWINARF
SKSGKPPINDMFTDLKDGRKLLDLLEGLTGTSLPKERGSTRVHAL
NNVNRVLQVLHQNNVELVNIGGTDIVDGNHKLTLGLLWSIILHWQ
VKDVMKDVMSDLQQTNSEKILLSWVRQTTRPYSQVNVLNFTTSWT
DGLAFNAVLHRHKPDLFSWDKVVKMSPIERLEHAFSKAQTYLGIE
KLLDPEDVAVQLPDKKSIIMYLTSLFEVLPQQVTIDAIREVETLP
RKYKKECEEEAINIQSTAPEEEHESPRAETPSTVTEVDMDLDSYQ
IALEEVLTWLLSAEDTFQEQDDISDDVEEVKDQFATHEAFMMELT
AHQSSVGSVLQAGNQLITQGTLSDEEEFEIQEQMTLLNARWEALR
VESMDRQSRLHDVLMELQKKQLQQLSAWLTLTEERIQKMETCPLD
DDVKSLQKLLEEHKSLQSDLEAEQVKVNSLTHMVVIVDENSGESA
TAILEDQLQKLGERWTAVCRWTEERWNRLQEIAASARFDRSVEKW
RRFHYDIKIFNQWLTEAEQFLRKTQIPENWEHAKYKWYLKELQDG
IGQRQTVVRTLNATGEEIIQQSSKTDASILQEKLGSLNLRWQEVC
KQLSDRKKRLEEQKQPDLAPGLTTIGASPTQTVTLVTQPVVTKET
AISKLEMPSSLMLEVPEETHRLLQQFPLDLEKFLAWLTEAETTAN
VLQDATRKERLLEDSKGVKELMKQWQDLQGEIEAHTDVYHNLDEN
SQKILRSLEGSDDAVLLQRRLDNMNFKWSELRKKSLNIRSHLEAS
SDQWKRLGPASQHELSTSVQGPWERAISPNKVPYYINHETQTTCW
DHPKMTELYQSLADLNNVRESAYRTAMKLRRLQKALCLDLLSLSA
ACDALDQHNLKQNDQPMDILQIINCLTTIYDRLEQEHNNLVNVPL
CVDMCLNWLLNVYDTGRIGRIRVLSFKTGIISLCKAHLEDKYRYL
FKQVASSTGFCDQRRLGLLLHDSIQIPRQLGEVASFGGSNIEPSV
RSCFQFANNKPEIEAALFLDWMRLEPQSMVWLPVLHRVAAAETAK
HQAKCNICKECPIIGFRYRSLKHFNYDICQSCFFSGRVAKGHKMH
YPMVEYCTPTTSGEDVRDFAKVLKNKFRTKRYFAKHPRMGYLPVQ
TVLEGDNMETPVTLINFWPVDSAPASSPQLSHDDTHSRIEHYASR
LAEMENSNGSYLNDSISPNESIDDEHLLIQHYCQSLNQDSPLSQP
RSPAQILISLESEERGELERILADLEEENRNLQAEYDRLKQQHEH
KGLSPLPSPPEMMPTSPQSP
UtroNterm-R1_DystroR17_H3_R21-R22_H4-
trCterm + SynBS_coiled coil
(SEQ ID NO: 164)
MAKYGEHEASPDNGQNEFSDIIKSRSDEHNDVQKKTFTKWINARF
SKSGKPPINDMFTDLKDGRKLLDLLEGLTGTSLPKERGSTRVHAL
NNVNRVLQVLHQNNVELVNIGGTDIVDGNHKLTLGLLWSIILHWQ
VKDVMKDVMSDLQQTNSEKILLSWVRQTTRPYSQVNVLNFTTSWT
DGLAFNAVLHRHKPDLFSWDKVVKMSPIERLEHAFSKAQTYLGIE
KLLDPEDVAVQLPDKKSIIMYLTSLFEVLPQQVTIDAIREVETLP
RKYKKECEEEAINIQSTAPEEEHESPRAETPSTVTEVDMDLDSYQ
IALEEVLTWLLSAEDTFQEQDDISDDVEEVKDQFATHEAFMMELT
AHQSSVGSVLQAGNQLITQGTLSDEEEFEIQEQMTLLNARWEALR
VESMDRQSRLHDVLMAASARFDRSVEKWRRFHYDIKIFNQWLTEA
EQFLRKTQIPENWEHAKYKWYLKELQDGIGQRQTVVRTLNATGEE
IIQQSSKTDASILQEKLGSLNLRWQEVCKQLSDRKKRLEEQKQPD
LAPGLITIGASPTQTVTLVTQPVVTKETAISKLEMPSSLMLEVPK
DSTQWLEAKEEAEQVLGQARAKLESWKEGPYTVDAIQKKITETKQ
LAKDLRQWQTNVDVANDLALKLLRDYSADDTRKVHMITENINASW
RSIHKRVSEREAALEETHRLLQQFPLDLEKFLAWLTEAETTANVL
QDATRKERLLEDSKGVKELMKQWQDLQGEIEAHTDVYHNLDENSQ
KILRSLEGSDDAVLLQRRLDNMNFKWSELRKKSLNIRSHLEASSD
QWKRLGPASQHELSTSVQGPWERAISPNKVPYYINHETQTTCWDH
PKMTELYQSLADLNNVRFSAYRTAMKLRRLQKALCLDLLSLSAAC
DALDQHNLKQNDQPMDILQIINCLITIYDRLEQEHNNLVNVPLCV
DMCLNWLLNVYDTGRIGRIRVLSFKTGIISLCKAHLEDKYRYLFK
QVASSTGFCDQRRLGLLLHDSIQIPRQLGEVASFGGSNIEPSVRS
CFQFANNKPEIEAALFLDWMRLEPQSMVWLPVLHRVAAAETAKHQ
AKCNICKECPIIGFRYRSLKHFNYDICQSCFFSGRVAKGHKMHYP
MVEYCTPTTSGEDVRDFAKVLKNKFRTKRYFAKHPRMGYLPVQTV
LEGDNMETPVTLINFWPVDSAPASSPQLSHDDTHSRIEHYASRLA
EMENSNGSYLNDSISPNESIDDEHLLIQHYCQSLNQDSPLSQPRS
PAQILISLESEERGELERILADLEEENRNLQAEYDRLKQQHEHKG
LSPLPSPPEMMPTSPQSP
UtroNterm-R2_DystroR17_ R21-R22_H4-
trCterm + SynBS
(SEQ ID NO: 165)
MAKYGEHEASPDNGQNEFSDIIKSRSDEHNDVQKKTFTKWINARF
SKSGKPPINDMFTDLKDGRKLLDLLEGLTGTSLPKERGSTRVHAL
NNVNRVLQVLHQNNVELVNIGGTDIVDGNHKLTLGLLWSIILHWQ
VKDVMKDVMSDLQQTNSEKILLSWVRQTTRPYSQVNVLNFTTSWT
DGLAFNAVLHRHKPDLFSWDKVVKMSPIERLEHAFSKAQTYLGIE
KLLDPEDVAVQLPDKKSIIMYLTSLFEVLPQQVTIDAIREVETLP
RKYKKECEEEAINIQSTAPEEEHESPRAETPSTVTEVDMDLDSYQ
IALEEVLTWLLSAEDTFQEQDDISDDVEEVKDQFATHEAFMMELT
AHQSSVGSVLQAGNQLITQGTLSDEEEFEIQEQMTLLNARWEALR
VESMDRQSRLHDVLMELQKKQLQQLSAWLTLTEERIQKMETCPLD
DDVKSLQKLLEEHKSLQSDLEAEQVKVNSLTHMVVIVDENSGESA
TAILEDQLQKLGERWTAVCRWTEERWNRLQEIAASARFDRSVEKW
RRFHYDIKIFNQWLTEAEQFLRKTQIPENWEHAKYKWYLKELQDG
IGQRQTVVRTLNATGEEIIQQSSKTDASILQEKLGSLNLRWQEVC
KQLSDRKKRLEEQKKDSTQWLEAKEEAEQVLGQARAKLESWKEGP
YTVDAIQKKITETKQLAKDLRQWQTNVDVANDLALKLLRDYSADD
TRKVHMITENINASWRSIHKRVSEREAALEETHRLLQQFPLDLEK
FLAWLTEAETTANVLQDATRKERLLEDSKGVKELMKQWQDLQGEI
EAHTDVYHNLDENSQKILRSLEGSDDAVLLQRRLDNMNFKWSELR
KKSLNIRSHLEASSDQWKRLGPASQHELSTSVQGPWERAISPNKV
PYYINHETQTTCWDHPKMTELYQSLADLNNVRFSAYRTAMKLRRL
QKALCLDLLSLSAACDALDQHNLKQNDQPMDILQIINCLTTIYDR
LEQEHNNLVNVPLCVDMCLNWLLNVYDTGRIGRIRVLSFKTGIIS
LCKAHLEDKYRYLFKQVASSTGFCDQRRLGLLLHDSIQIPRQLGE
VASFGGSNIEPSVRSCFQFANNKPEIEAALFLDWMRLEPQSMVWL
PVLHRVAAAETAKHQAKCNICKECPIIGFRYRSLKHFNYDICQSC
FFSGRVAKGHKMHYPMVEYCTPTTSGEDVRDFAKVLKNKFRTKRY
FAKHPRMGYLPVQTVLEGDNMETPVTLINFWPVDSAPASSPQLSH
DDTHSRIEHYASRLAEMENSNGSYLNDSISPNESIDDEHLLIQHY
CQSLNQDSPLSQPRS

Non-limiting examples of nucleotide sequences encoding chimeric utrophin/dystrophin micro-dystrophins for use in both heart and skeletal muscle are provided below.

UtrophinNterm-R2_DystrophinR17_H3_R22_
H4-trCterm + SynBS
Human sequence without codon alterations:
(SEQ ID NO: 176)
ATGGCCAAGTATGGAGAACATGAAGCCAGTCCTGACAATGGGCAG
AACGAATTCAGTGATATCATTAAGTCCAGATCTGATGAACACAAT
GACGTACAGAAGAAAACCTTTACCAAATGGATAAATGCTCGATTT
TCAAAGAGTGGGAAACCACCCATCAATGATATGTTCACAGACCTC
AAAGATGGAAGGAAGCTATTGGATCTTCTAGAAGGCCTCACAGGA
ACATCACTGCCAAAGGAACGTGGTTCCACAAGGGTACATGCCTTA
AATAACGTCAACAGAGTGCTGCAGGTTTTACATCAGAACAATGTG
GAATTAGTGAATATAGGGGGAACTGACATTGTGGATGGAAATCAC
AAACTGACTTTGGGGTTACTTTGGAGCATCATTTTGCACTGGCAG
GTGAAAGATGTCATGAAGGATGTCATGTCGGACCTGCAGCAGACG
AACAGTGAGAAGATCCTGCTCAGCTGGGTGCGTCAGACCACCAGG
CCCTACAGCCAAGTCAACGTCCTCAACTTCACCACCAGCTGGACA
GATGGACTCGCCTTTAATGCTGTCCTCCACCGACATAAACCTGAT
CTCTTCAGCTGGGATAAAGTTGTCAAAATGTCACCAATTGAGAGA
CTTGAACATGCCTTCAGCAAGGCTCAAACTTATTTGGGAATTGAA
AAGCTGTTAGATCCTGAAGATGTTGCCGTTCAGCTTCCTGACAAG
AAATCCATAATTATGTATTTAACATCTTTGTTTGAGGTGCTACCT
CAGCAAGTCACCATAGACGCCATCCGTGAGGTAGAGACACTCCCA
AGGAAATATAAAAAAGAATGTGAAGAAGAGGCAATTAATATACAG
AGTACAGCGCCTGAGGAGGAGCATGAGAGTCCCCGAGCTGAAACT
CCCAGCACTGTCACTGAGGTTGACATGGATCTGGACAGCTATCAG
ATTGCGTTGGAGGAAGTGCTGACCTGGTTGCTTTCTGCTGAGGAC
ACTTTCCAGGAGCAGGATGATATTTCTGATGATGTTGAAGAAGTC
AAAGACCAGTTTGCAACCCATGAAGCTTTTATGATGGAACTGACT
GCACACCAGAGCAGTGTGGGCAGCGTCCTGCAGGCAGGCAACCAA
CTGATAACACAAGGAACTCTGTCAGACGAAGAAGAATTTGAGATT
CAGGAACAGATGACCCTGCTGAATGCTAGATGGGAGGCTCTTAGG
GTGGAGAGTATGGACAGACAGTCCCGGCTGCACGATGTGCTGATG
GAACTGCAGAAGAAGCAACTGCAGCAGCTCTCCGCCTGGTTAACA
CTCACAGAGGAGCGCATTCAGAAGATGGAAACTTGCCCCCTGGAT
GATGATGTAAAATCTCTACAAAAGCTGCTAGAAGAACATAAAAGT
TTGCAAAGTGATCTTGAGGCTGAACAGGTGAAAGTAAATTCACTA
ACTCACATGGTGGTCATTGTTGATGAAAACAGTGGTGAGAGTGCT
ACAGCTATCCTAGAAGACCAGTTACAGAAACTTGGTGAGCGCTGG
ACAGCAGTATGCCGTTGGACTGAAGAACGCTGGAATAGGTTACAA
GAAATCGCCGCTAGCGCCCGATTTGACAGATCTGTTGAGAAATGG
CGGCGTTTTCATTATGATATAAAGATATTTAATCAGTGGCTAACA
GAAGCTGAACAGTTTCTCAGAAAGACACAAATTCCTGAGAATTGG
GAACATGCTAAATACAAATGGTATCTTAAGGAACTCCAGGATGGC
ATTGGGCAGCGGCAAACTGTTGTCAGAACATTGAATGCAACTGGG
GAAGAAATAATTCAGCAATCCTCAAAAACAGATGCCAGTATTCTA
CAGGAAAAATTGGGAAGCCTGAATCTGCGGTGGCAGGAGGTCTGC
AAACAGCTGTCAGACAGAAAAAAGAGGCTAGAAGAACAAAAGCAG
CCTGACCTAGCTCCTGGACTGACCACTATTGGAGCCTCTCCTACT
CAGACTGTTACTCTGGTGACACAACCTGTGGTTACTAAGGAAACT
GCCATCTCCAAACTAGAAATGCCATCTTCCTTGATGTTGGAGGTA
CCTGAAGAAACTCATAGATTACTGCAACAGTTCCCCCTGGACCTG
GAAAAGTTTCTTGCCTGGCTTACAGAAGCTGAAACAACTGCCAAT
GTCCTACAGGATGCTACCCGTAAGGAAAGGCTCCTAGAAGACTCC
AAGGGAGTAAAAGAGCTGATGAAACAATGGCAAGACCTCCAAGGT
GAAATTGAAGCTCACACAGATGTTTATCACAACCTGGATGAAAAC
AGCCAAAAAATCCTGAGATCCCTGGAAGGTTCCGATGATGCAGTC
CTGTTACAAAGACGTTTGGATAACATGAACTTCAAGTGGAGTGAA
CTTCGGAAAAAGTCTCTCAACATTAGGTCCCATTTGGAAGCCAGT
TCTGACCAGTGGAAGCGTCTGGGTCCAGCATCTCAGCACTTTCTT
TCCACGTCTGTCCAGGGTCCCTGGGAGAGAGCCATCTCGCCAAAC
AAAGTGCCCTACTATATCAACCACGAGACTCAAACAACTTGCTGG
GACCATCCCAAAATGACAGAGCTCTACCAGTCTTTAGCTGACCTG
AATAATGTCAGATTCTCAGCTTATAGGACTGCCATGAAACTCCGA
AGACTGCAGAAGGCCCTTTGCTTGGATCTCTTGAGCCTGTCAGCT
GCATGTGATGCCTTGGACCAGCACAACCTCAAGCAAAATGACCAG
CCCATGGATATCCTGCAGATTATTAATTGTTTGACCACTATTTAT
GACCGCCTGGAGCAAGAGCACAACAATTTGGTCAACGTCCCTCTC
TGCGTGGATATGTGTCTGAACTGGCTGCTGAATGTTTATGATACG
GGACGAACAGGGAGGATCCGTGTCCTGTCTTTTAAAACTGGCATC
ATTTCCCTGTGTAAAGCACATTTGGAAGACAAGTACAGATACCTT
TTCAAGCAAGTGGCAAGTTCAACAGGATTTTGTGACCAGCGCAGG
CTGGGCCTCCTTCTGCATGATTCTATCCAAATTCCAAGACAGTTG
GGTGAAGTTGCATCCTTTGGGGGCAGTAACATTGAGCCAAGTGTC
CGGAGCTGCTTCCAATTTGCTAATAATAAGCCAGAGATCGAAGCG
GCCCTCTTCCTAGACTGGATGAGACTGGAACCCCAGTCCATGGTG
TGGCTGCCCGTCCTGCACAGAGTGGCTGCTGCAGAAACTGCCAAG
CATCAGGCCAAATGTAACATCTGCAAAGAGTGTCCAATCATTGGA
TTCAGGTACAGGAGTCTAAAGCACTTTAATTATGACATCTGCCAA
AGCTGCTTTTTTTCTGGTCGAGTTGCAAAAGGCCATAAAATGCAC
TATCCCATGGTGGAATATTGCACTCCGACTACATCAGGAGAAGAT
GTTCGAGACTTTGCCAAGGTACTAAAAAACAAATTTCGAACCAAA
AGGTATTTTGCGAAGCATCCCCGAATGGGCTACCTGCCAGTGCAG
ACTGTCTTAGAGGGGGACAACATGGAAACTCCCGTTACTCTGATC
AACTTCTGGCCAGTAGATTCTGCGCCTGCCTCGTCCCCTCAGCTT
TCACACGATGATACTCATTCACGCATTGAACATTATGCTAGCAGG
CTAGCAGAAATGGAAAACAGCAATGGATCTTATCTAAATGATAGC
ATCTCTCCTAATGAGAGCATAGATGATGAACATTTGTTAATCCAG
CATTACTGCCAAAGTTTGAACCAGGACTCCCCCCTGAGCCAGCCT
CGTAGT
Human sequence with codon optimization:
(SEQ ID NO: 177)
ATGGCCAAGTACGGCGAGCACGAGGCCAGCCCCGACAACGGCCAG
AACGAGTTCAGCGACATCATCAAGAGCCGGAGCGACGAGCACAAC
GACGTGCAGAAGAAGACCTTCACCAAGTGGATCAACGCCCGGTTC
AGCAAGAGCGGCAAGCCCCCCATCAACGACATGTTCACCGACCTG
AAGGACGGCCGGAAGCTGCTGGACCTGCTGGAGGGCCTGACCGGC
ACCAGCCTGCCCAAGGAGCGGGGCAGCACCCGGGTGCACGCCCTG
AACAACGTGAACCGGGTGCTGCAGGTGCTGCACCAGAACAACGTG
GAGCTGGTGAACATCGGCGGCACCGACATCGTGGACGGCAACCAC
AAGCTGACCCTGGGCCTGCTGTGGAGCATCATCCTGCACTGGCAG
GTGAAGGACGTGATGAAGGACGTGATGAGCGACCTGCAGCAGACC
AACAGCGAGAAGATCCTGCTGAGCTGGGTGCGGCAGACCACCCGG
CCCTACAGCCAGGTGAACGTGCTGAACTTCACCACCAGCTGGACC
GACGGCCTGGCCTTCAACGCCGTGCTGCACCGGCACAAGCCCGAC
CTGTTCAGCTGGGACAAGGTGGTGAAGATGAGCCCCATCGAGCGG
CTGGAGCACGCCTTCAGCAAGGCCCAGACCTACCTGGGCATCGAG
AAGCTGCTGGACCCCGAGGACGTGGCCGTGCAGCTGCCCGACAAG
AAGAGCATCATCATGTACCTGACCAGCCTGTTCGAGGTGCTGCCC
CAGCAGGTGACCATCGACGCCATCCGGGAGGTGGAGACCCTGCCC
CGGAAGTACAAGAAGGAGTGCGAGGAGGAGGCCATCAACATCCAG
AGCACCGCCCCCGAGGAGGAGCACGAGAGCCCCCGGGCCGAGACC
CCCAGCACCGTGACCGAGGTGGACATGGACCTGGACAGCTACCAG
ATCGCCCTGGAGGAGGTGCTGACCTGGCTGCTGAGCGCCGAGGAC
ACCTTCCAGGAGCAGGACGACATCAGCGACGACGTGGAGGAGGTG
AAGGACCAGTTCGCCACCCACGAGGCCTTCATGATGGAGCTGACC
GCCCACCAGAGCAGCGTGGGCAGCGTGCTGCAGGCCGGCAACCAG
CTGATCACCCAGGGCACCCTGAGCGACGAGGAGGAGTTCGAGATC
CAGGAGCAGATGACCCTGCTGAACGCCCGGTGGGAGGCCCTGCGG
GTGGAGAGCATGGACCGGCAGAGCCGGCTGCACGACGTGCTGATG
GAGCTGCAGAAGAAGCAGCTGCAGCAGCTGAGCGCCTGGCTGACC
CTGACCGAGGAGCGGATCCAGAAGATGGAGACCTGCCCCCTGGAC
GACGACGTGAAGAGCCTGCAGAAGCTGCTGGAGGAGCACAAGAGC
CTGCAGAGCGACCTGGAGGCCGAGCAGGTGAAGGTGAACAGCCTG
ACCCACATGGTGGTGATCGTGGACGAGAACAGCGGCGAGAGCGCC
ACCGCCATCCTGGAGGACCAGCTGCAGAAGCTGGGCGAGCGGTGG
ACCGCCGTGTGCCGGTGGACCGAGGAGCGGTGGAACCGGCTGCAG
GAGATCGCCGCTAGCGCCCGGTTCGACCGGAGCGTGGAGAAGTGG
CGGCGGTTCCACTACGACATCAAGATCTTCAACCAGTGGCTGACC
GAGGCCGAGCAGTTCCTGCGGAAGACCCAGATCCCCGAGAACTGG
GAGCACGCCAAGTACAAGTGGTACCTGAAGGAGCTGCAGGACGGC
ATCGGCCAGCGGCAGACCGTGGTGCGGACCCTGAACGCCACCGGC
GAGGAGATCATCCAGCAGAGCAGCAAGACCGACGCCAGCATCCTG
CAGGAGAAGCTGGGCAGCCTGAACCTGCGGTGGCAGGAGGTGTGC
AAGCAGCTGAGCGACCGGAAGAAGCGGCTGGAGGAGCAGAAGCAG
CCCGACCTGGCCCCCGGCCTGACCACCATCGGCGCCAGCCCCACC
CAGACCGTGACCCTGGTGACCCAGCCCGTGGTGACCAAGGAGACC
GCCATCAGCAAGCTGGAGATGCCCAGCAGCCTGATGCTGGAGGTG
CCCGAGGAGACCCACCGGCTGCTGCAGCAGTTCCCCCTGGACCTG
GAGAAGTTCCTGGCCTGGCTGACCGAGGCCGAGACCACCGCCAAC
GTGCTGCAGGACGCCACCCGGAAGGAGCGGCTGCTGGAGGACAGC
AAGGGCGTGAAGGAGCTGATGAAGCAGTGGCAGGACCTGCAGGGC
GAGATCGAGGCCCACACCGACGTGTACCACAACCTGGACGAGAAC
AGCCAGAAGATCCTGCGGAGCCTGGAGGGCAGCGACGACGCCGTG
CTGCTGCAGCGGCGGCTGGACAACATGAACTTCAAGTGGAGCGAG
CTGCGGAAGAAGAGCCTGAACATCCGGAGCCACCTGGAGGCCAGC
AGCGACCAGTGGAAGCGGCTGGGCCCCGCCAGCCAGCACTTCCTG
AGCACCAGCGTGCAGGGCCCCTGGGAGCGGGCCATCAGCCCCAAC
AAGGTGCCCTACTACATCAACCACGAGACCCAGACCACCTGCTGG
GACCACCCCAAGATGACCGAGCTGTACCAGAGCCTGGCCGACCTG
AACAACGTGCGGTTCAGCGCCTACCGGACCGCCATGAAGCTGCGG
CGGCTGCAGAAGGCCCTGTGCCTGGACCTGCTGAGCCTGAGCGCC
GCCTGCGACGCCCTGGACCAGCACAACCTGAAGCAGAACGACCAG
CCCATGGACATCCTGCAGATCATCAACTGCCTGACCACCATCTAC
GACCGGCTGGAGCAGGAGCACAACAACCTGGTGAACGTGCCCCTG
TGCGTGGACATGTGCCTGAACTGGCTGCTGAACGTGTACGACACC
GGCCGGACCGGCCGGATCCGGGTGCTGAGCTTCAAGACCGGCATC
ATCAGCCTGTGCAAGGCCCACCTGGAGGACAAGTACCGGTACCTG
TTCAAGCAGGTGGCCAGCAGCACCGGCTTCTGCGACCAGCGGCGG
CTGGGCCTGCTGCTGCACGACAGCATCCAGATCCCCCGGCAGCTG
GGCGAGGTGGCCAGCTTCGGCGGCAGCAACATCGAGCCCAGCGTG
CGGAGCTGCTTCCAGTTCGCCAACAACAAGCCCGAGATCGAGGCC
GCCCTGTTCCTGGACTGGATGCGGCTGGAGCCCCAGAGCATGGTG
TGGCTGCCCGTGCTGCACCGGGTGGCCGCCGCCGAGACCGCCAAG
CACCAGGCCAAGTGCAACATCTGCAAGGAGTGCCCCATCATCGGC
TTCCGGTACCGGAGCCTGAAGCACTTCAACTACGACATCTGCCAG
AGCTGCTTCTTCAGCGGCCGGGTGGCCAAGGGCCACAAGATGCAC
TACCCCATGGTGGAGTACTGCACCCCCACCACCAGCGGCGAGGAC
GTGCGGGACTTCGCCAAGGTGCTGAAGAACAAGTTCCGGACCAAG
CGGTACTTCGCCAAGCACCCCCGGATGGGCTACCTGCCCGTGCAG
ACCGTGCTGGAGGGCGACAACATGGAGACCCCCGTGACCCTGATC
AACTTCTGGCCCGTGGACAGCGCCCCCGCCAGCAGCCCCCAGCTG
AGCCACGACGACACCCACAGCCGGATCGAGCACTACGCCAGCCGG
CTGGCCGAGATGGAGAACAGCAACGGCAGCTACCTGAACGACAGC
ATCAGCCCCAACGAGAGCATCGACGACGAGCACCTGCTGATCCAG
CACTACTGCCAGAGCCTGAACCAGGACAGCCCCCTGAGCCAGCCC
CGGAGC
UtrophinNterm-R1_DystrophinR17_H3_R21-
R22_H4-trCterm + SynBS
Human sequence without codon alterations:
(SEQ ID NO: 178)
ATGGCCAAGTATGGAGAACATGAAGCCAGTCCTGACAATGGGCAG
AACGAATTCAGTGATATCATTAAGTCCAGATCTGATGAACACAAT
GACGTACAGAAGAAAACCTTTACCAAATGGATAAATGCTCGATTT
TCAAAGAGTGGGAAACCACCCATCAATGATATGTTCACAGACCTC
AAAGATGGAAGGAAGCTATTGGATCTTCTAGAAGGCCTCACAGGA
ACATCACTGCCAAAGGAACGTGGTTCCACAAGGGTACATGCCTTA
AATAACGTCAACAGAGTGCTGCAGGTTTTACATCAGAACAATGTG
GAATTAGTGAATATAGGGGGAACTGACATTGTGGATGGAAATCAC
AAACTGACTTTGGGGTTACTTTGGAGCATCATTTTGCACTGGCAG
GTGAAAGATGTCATGAAGGATGTCATGTCGGACCTGCAGCAGACG
AACAGTGAGAAGATCCTGCTCAGCTGGGTGCGTCAGACCACCAGG
CCCTACAGCCAAGTCAACGTCCTCAACTTCACCACCAGCTGGACA
GATGGACTCGCCTTTAATGCTGTCCTCCACCGACATAAACCTGAT
CTCTTCAGCTGGGATAAAGTTGTCAAAATGTCACCAATTGAGAGA
CTTGAACATGCCTTCAGCAAGGCTCAAACTTATTTGGGAATTGAA
AAGCTGTTAGATCCTGAAGATGTTGCCGTTCAGCTTCCTGACAAG
AAATCCATAATTATGTATTTAACATCTTTGTTTGAGGTGCTACCT
CAGCAAGTCACCATAGACGCCATCCGTGAGGTAGAGACACTCCCA
AGGAAATATAAAAAAGAATGTGAAGAAGAGGCAATTAATATACAG
AGTACAGCGCCTGAGGAGGAGCATGAGAGTCCCCGAGCTGAAACT
CCCAGCACTGTCACTGAGGTTGACATGGATCTGGACAGCTATCAG
ATTGCGTTGGAGGAAGTGCTGACCTGGTTGCTTTCTGCTGAGGAC
ACTTTCCAGGAGCAGGATGATATTTCTGATGATGTTGAAGAAGTC
AAAGACCAGTTTGCAACCCATGAAGCTTTTATGATGGAACTGACT
GCACACCAGAGCAGTGTGGGCAGCGTCCTGCAGGCAGGCAACCAA
CTGATAACACAAGGAACTCTGTCAGACGAAGAAGAATTTGAGATT
CAGGAACAGATGACCCTGCTGAATGCTAGATGGGAGGCTCTTAGG
GTGGAGAGTATGGACAGACAGTCCCGGCTGCACGATGTGCTGATG
GCCGCTAGCGCCCGATTTGACAGATCTGTTGAGAAATGGCGGCGT
TTTCATTATGATATAAAGATATTTAATCAGTGGCTAACAGAAGCT
GAACAGTTTCTCAGAAAGACACAAATTCCTGAGAATTGGGAACAT
GCTAAATACAAATGGTATCTTAAGGAACTCCAGGATGGCATTGGG
CAGCGGCAAACTGTTGTCAGAACATTGAATGCAACTGGGGAAGAA
ATAATTCAGCAATCCTCAAAAACAGATGCCAGTATTCTACAGGAA
AAATTGGGAAGCCTGAATCTGCGGTGGCAGGAGGTCTGCAAACAG
CTGTCAGACAGAAAAAAGAGGCTAGAAGAACAAAAGCAGCCTGAC
CTAGCTCCTGGACTGACCACTATTGGAGCCTCTCCTACTCAGACT
GTTACTCTGGTGACACAACCTGTGGTTACTAAGGAAACTGCCATC
TCCAAACTAGAAATGCCATCTTCCTTGATGTTGGAGGTACCTAAG
GATTCAACACAATGGCTGGAAGCTAAGGAAGAAGCTGAGCAGGTC
TTAGGACAGGCCAGAGCCAAGCTTGAGTCATGGAAGGAGGGTCCC
TATACAGTAGATGCAATCCAAAAGAAAATCACAGAAACCAAGCAG
TTGGCCAAAGACCTCCGCCAGTGGCAGACAAATGTAGATGTGGCA
AATGACTTGGCCCTGAAACTTCTCCGGGATTATTCTGCAGATGAT
ACCAGAAAAGTCCACATGATAACAGAGAATATCAATGCCTCTTGG
AGAAGCATTCATAAAAGGGTGAGTGAGCGAGAGGCTGCTTTGGAA
GAAACTCATAGATTACTGCAACAGTTCCCCCTGGACCTGGAAAAG
TTTCTTGCCTGGCTTACAGAAGCTGAAACAACTGCCAATGTCCTA
CAGGATGCTACCCGTAAGGAAAGGCTCCTAGAAGACTCCAAGGGA
GTAAAAGAGCTGATGAAACAATGGCAAGACCTCCAAGGTGAAATT
GAAGCTCACACAGATGTTTATCACAACCTGGATGAAAACAGCCAA
AAAATCCTGAGATCCCTGGAAGGTTCCGATGATGCAGTCCTGTTA
CAAAGACGTTTGGATAACATGAACTTCAAGTGGAGTGAACTTCGG
AAAAAGTCTCTCAACATTAGGTCCCATTTGGAAGCCAGTTCTGAC
CAGTGGAAGCGTCTGGGTCCAGCATCTCAGCACTTTCTTTCCACG
TCTGTCCAGGGTCCCTGGGAGAGAGCCATCTCGCCAAACAAAGTG
CCCTACTATATCAACCACGAGACTCAAACAACTTGCTGGGACCAT
CCCAAAATGACAGAGCTCTACCAGTCTTTAGCTGACCTGAATAAT
GTCAGATTCTCAGCTTATAGGACTGCCATGAAACTCCGAAGACTG
CAGAAGGCCCTTTGCTTGGATCTCTTGAGCCTGTCAGCTGCATGT
GATGCCTTGGACCAGCACAACCTCAAGCAAAATGACCAGCCCATG
GATATCCTGCAGATTATTAATTGTTTGACCACTATTTATGACCGC
CTGGAGCAAGAGCACAACAATTTGGTCAACGTCCCTCTCTGCGTG
GATATGTGTCTGAACTGGCTGCTGAATGTTTATGATACGGGACGA
ACAGGGAGGATCCGTGTCCTGTCTTTTAAAACTGGCATCATTTCC
CTGTGTAAAGCACATTTGGAAGACAAGTACAGATACCTTTTCAAG
CAAGTGGCAAGTTCAACAGGATTTTGTGACCAGCGCAGGCTGGGC
CTCCTTCTGCATGATTCTATCCAAATTCCAAGACAGTTGGGTGAA
GTTGCATCCTTTGGGGGCAGTAACATTGAGCCAAGTGTCCGGAGC
TGCTTCCAATTTGCTAATAATAAGCCAGAGATCGAAGCGGCCCTC
TTCCTAGACTGGATGAGACTGGAACCCCAGTCCATGGTGTGGCTG
CCCGTCCTGCACAGAGTGGCTGCTGCAGAAACTGCCAAGCATCAG
GCCAAATGTAACATCTGCAAAGAGTGTCCAATCATTGGATTCAGG
TACAGGAGTCTAAAGCACTTTAATTATGACATCTGCCAAAGCTGC
TTTTTTTCTGGTCGAGTTGCAAAAGGCCATAAAATGCACTATCCC
ATGGTGGAATATTGCACTCCGACTACATCAGGAGAAGATGTTCGA
GACTTTGCCAAGGTACTAAAAAACAAATTTCGAACCAAAAGGTAT
TTTGCGAAGCATCCCCGAATGGGCTACCTGCCAGTGCAGACTGTC
TTAGAGGGGGACAACATGGAAACTCCCGTTACTCTGATCAACTTC
TGGCCAGTAGATTCTGCGCCTGCCTCGTCCCCTCAGCTTTCACAC
GATGATACTCATTCACGCATTGAACATTATGCTAGCAGGCTAGCA
GAAATGGAAAACAGCAATGGATCTTATCTAAATGATAGCATCTCT
CCTAATGAGAGCATAGATGATGAACATTTGTTAATCCAGCATTAC
TGCCAAAGTTTGAACCAGGACTCCCCCCTGAGCCAGCCTCGTAGT
Human sequence with codon optimization:
(SEQ ID NO: 179)
ATGGCCAAGTACGGCGAGCACGAGGCCAGCCCCGACAACGGCCAG
AACGAGTTCAGCGACATCATCAAGAGCCGGAGCGACGAGCACAAC
GACGTGCAGAAGAAGACCTTCACCAAGTGGATCAACGCCCGGTTC
AGCAAGAGCGGCAAGCCCCCCATCAACGACATGTTCACCGACCTG
AAGGACGGCCGGAAGCTGCTGGACCTGCTGGAGGGCCTGACCGGC
ACCAGCCTGCCCAAGGAGCGGGGCAGCACCCGGGTGCACGCCCTG
AACAACGTGAACCGGGTGCTGCAGGTGCTGCACCAGAACAACGTG
GAGCTGGTGAACATCGGCGGCACCGACATCGTGGACGGCAACCAC
AAGCTGACCCTGGGCCTGCTGTGGAGCATCATCCTGCACTGGCAG
GTGAAGGACGTGATGAAGGACGTGATGAGCGACCTGCAGCAGACC
AACAGCGAGAAGATCCTGCTGAGCTGGGTGCGGCAGACCACCCGG
CCCTACAGCCAGGTGAACGTGCTGAACTTCACCACCAGCTGGACC
GACGGCCTGGCCTTCAACGCCGTGCTGCACCGGCACAAGCCCGAC
CTGTTCAGCTGGGACAAGGTGGTGAAGATGAGCCCCATCGAGCGG
CTGGAGCACGCCTTCAGCAAGGCCCAGACCTACCTGGGCATCGAG
AAGCTGCTGGACCCCGAGGACGTGGCCGTGCAGCTGCCCGACAAG
AAGAGCATCATCATGTACCTGACCAGCCTGTTCGAGGTGCTGCCC
CAGCAGGTGACCATCGACGCCATCCGGGAGGTGGAGACCCTGCCC
CGGAAGTACAAGAAGGAGTGCGAGGAGGAGGCCATCAACATCCAG
AGCACCGCCCCCGAGGAGGAGCACGAGAGCCCCCGGGCCGAGACC
CCCAGCACCGTGACCGAGGTGGACATGGACCTGGACAGCTACCAG
ATCGCCCTGGAGGAGGTGCTGACCTGGCTGCTGAGCGCCGAGGAC
ACCTTCCAGGAGCAGGACGACATCAGCGACGACGTGGAGGAGGTG
AAGGACCAGTTCGCCACCCACGAGGCCTTCATGATGGAGCTGACC
GCCCACCAGAGCAGCGTGGGCAGCGTGCTGCAGGCCGGCAACCAG
CTGATCACCCAGGGCACCCTGAGCGACGAGGAGGAGTTCGAGATC
CAGGAGCAGATGACCCTGCTGAACGCCCGGTGGGAGGCCCTGCGG
GTGGAGAGCATGGACCGGCAGAGCCGGCTGCACGACGTGCTGATG
GCCGCTAGCGCCCGGTTCGACCGGAGCGTGGAGAAGTGGCGGCGG
TTCCACTACGACATCAAGATCTTCAACCAGTGGCTGACCGAGGCC
GAGCAGTTCCTGCGGAAGACCCAGATCCCCGAGAACTGGGAGCAC
GCCAAGTACAAGTGGTACCTGAAGGAGCTGCAGGACGGCATCGGC
CAGCGGCAGACCGTGGTGCGGACCCTGAACGCCACCGGCGAGGAG
ATCATCCAGCAGAGCAGCAAGACCGACGCCAGCATCCTGCAGGAG
AAGCTGGGCAGCCTGAACCTGCGGTGGCAGGAGGTGTGCAAGCAG
CTGAGCGACCGGAAGAAGCGGCTGGAGGAGCAGAAGCAGCCCGAC
CTGGCCCCCGGCCTGACCACCATCGGCGCCAGCCCCACCCAGACC
GTGACCCTGGTGACCCAGCCCGTGGTGACCAAGGAGACCGCCATC
AGCAAGCTGGAGATGCCCAGCAGCCTGATGCTGGAGGTGCCCAAG
GACAGCACCCAGTGGCTGGAGGCCAAGGAGGAGGCCGAGCAGGTG
CTGGGCCAGGCCCGGGCCAAGCTGGAGAGCTGGAAGGAGGGCCCC
TACACCGTGGACGCCATCCAGAAGAAGATCACCGAGACCAAGCAG
CTGGCCAAGGACCTGCGGCAGTGGCAGACCAACGTGGACGTGGCC
AACGACCTGGCCCTGAAGCTGCTGCGGGACTACAGCGCCGACGAC
ACCCGGAAGGTGCACATGATCACCGAGAACATCAACGCCAGCTGG
CGGAGCATCCACAAGCGGGTGAGCGAGCGGGAGGCCGCCCTGGAG
GAGACCCACCGGCTGCTGCAGCAGTTCCCCCTGGACCTGGAGAAG
TTCCTGGCCTGGCTGACCGAGGCCGAGACCACCGCCAACGTGCTG
CAGGACGCCACCCGGAAGGAGCGGCTGCTGGAGGACAGCAAGGGC
GTGAAGGAGCTGATGAAGCAGTGGCAGGACCTGCAGGGCGAGATC
GAGGCCCACACCGACGTGTACCACAACCTGGACGAGAACAGCCAG
AAGATCCTGCGGAGCCTGGAGGGCAGCGACGACGCCGTGCTGCTG
CAGCGGCGGCTGGACAACATGAACTTCAAGTGGAGCGAGCTGCGG
AAGAAGAGCCTGAACATCCGGAGCCACCTGGAGGCCAGCAGCGAC
CAGTGGAAGCGGCTGGGCCCCGCCAGCCAGCACTTCCTGAGCACC
AGCGTGCAGGGCCCCTGGGAGCGGGCCATCAGCCCCAACAAGGTG
CCCTACTACATCAACCACGAGACCCAGACCACCTGCTGGGACCAC
CCCAAGATGACCGAGCTGTACCAGAGCCTGGCCGACCTGAACAAC
GTGCGGTTCAGCGCCTACCGGACCGCCATGAAGCTGCGGCGGCTG
CAGAAGGCCCTGTGCCTGGACCTGCTGAGCCTGAGCGCCGCCTGC
GACGCCCTGGACCAGCACAACCTGAAGCAGAACGACCAGCCCATG
GACATCCTGCAGATCATCAACTGCCTGACCACCATCTACGACCGG
CTGGAGCAGGAGCACAACAACCTGGTGAACGTGCCCCTGTGCGTG
GACATGTGCCTGAACTGGCTGCTGAACGTGTACGACACCGGCCGG
ACCGGCCGGATCCGGGTGCTGAGCTTCAAGACCGGCATCATCAGC
CTGTGCAAGGCCCACCTGGAGGACAAGTACCGGTACCTGTTCAAG
CAGGTGGCCAGCAGCACCGGCTTCTGCGACCAGCGGCGGCTGGGC
CTGCTGCTGCACGACAGCATCCAGATCCCCCGGCAGCTGGGCGAG
GTGGCCAGCTTCGGCGGCAGCAACATCGAGCCCAGCGTGCGGAGC
TGCTTCCAGTTCGCCAACAACAAGCCCGAGATCGAGGCCGCCCTG
TTCCTGGACTGGATGCGGCTGGAGCCCCAGAGCATGGTGTGGCTG
CCCGTGCTGCACCGGGTGGCCGCCGCCGAGACCGCCAAGCACCAG
GCCAAGTGCAACATCTGCAAGGAGTGCCCCATCATCGGCTTCCGG
TACCGGAGCCTGAAGCACTTCAACTACGACATCTGCCAGAGCTGC
TTCTTCAGCGGCCGGGTGGCCAAGGGCCACAAGATGCACTACCCC
ATGGTGGAGTACTGCACCCCCACCACCAGCGGCGAGGACGTGCGG
GACTTCGCCAAGGTGCTGAAGAACAAGTTCCGGACCAAGCGGTAC
TTCGCCAAGCACCCCCGGATGGGCTACCTGCCCGTGCAGACCGTG
CTGGAGGGCGACAACATGGAGACCCCCGTGACCCTGATCAACTTC
TGGCCCGTGGACAGCGCCCCCGCCAGCAGCCCCCAGCTGAGCCAC
GACGACACCCACAGCCGGATCGAGCACTACGCCAGCCGGCTGGCC
GAGATGGAGAACAGCAACGGCAGCTACCTGAACGACAGCATCAGC
CCCAACGAGAGCATCGACGACGAGCACCTGCTGATCCAGCACTAC
TGCCAGAGCCTGAACCAGGACAGCCCCCTGAGCCAGCCCCGGAGC
UtrophinNterm-R2_DystrophinR17_H3_R22_
H4-trCterm + SynBS + coiled coil
Human sequence without codon alterations:
(SEQ ID NO: 180)
ATGGCCAAGTATGGAGAACATGAAGCCAGTCCTGACAATGGGCAG
AACGAATTCAGTGATATCATTAAGTCCAGATCTGATGAACACAAT
GACGTACAGAAGAAAACCTTTACCAAATGGATAAATGCTCGATTT
TCAAAGAGTGGGAAACCACCCATCAATGATATGTTCACAGACCTC
AAAGATGGAAGGAAGCTATTGGATCTTCTAGAAGGCCTCACAGGA
ACATCACTGCCAAAGGAACGTGGTTCCACAAGGGTACATGCCTTA
AATAACGTCAACAGAGTGCTGCAGGTTTTACATCAGAACAATGTG
GAATTAGTGAATATAGGGGGAACTGACATTGTGGATGGAAATCAC
AAACTGACTTTGGGGTTACTTTGGAGCATCATTTTGCACTGGCAG
GTGAAAGATGTCATGAAGGATGTCATGTCGGACCTGCAGCAGACG
AACAGTGAGAAGATCCTGCTCAGCTGGGTGCGTCAGACCACCAGG
CCCTACAGCCAAGTCAACGTCCTCAACTTCACCACCAGCTGGACA
GATGGACTCGCCTTTAATGCTGTCCTCCACCGACATAAACCTGAT
CTCTTCAGCTGGGATAAAGTTGTCAAAATGTCACCAATTGAGAGA
CTTGAACATGCCTTCAGCAAGGCTCAAACTTATTTGGGAATTGAA
AAGCTGTTAGATCCTGAAGATGTTGCCGTTCAGCTTCCTGACAAG
AAATCCATAATTATGTATTTAACATCTTTGTTTGAGGTGCTACCT
CAGCAAGTCACCATAGACGCCATCCGTGAGGTAGAGACACTCCCA
AGGAAATATAAAAAAGAATGTGAAGAAGAGGCAATTAATATACAG
AGTACAGCGCCTGAGGAGGAGCATGAGAGTCCCCGAGCTGAAACT
CCCAGCACTGTCACTGAGGTTGACATGGATCTGGACAGCTATCAG
ATTGCGTTGGAGGAAGTGCTGACCTGGTTGCTTTCTGCTGAGGAC
ACTTTCCAGGAGCAGGATGATATTTCTGATGATGTTGAAGAAGTC
AAAGACCAGTTTGCAACCCATGAAGCTTTTATGATGGAACTGACT
GCACACCAGAGCAGTGTGGGCAGCGTCCTGCAGGCAGGCAACCAA
CTGATAACACAAGGAACTCTGTCAGACGAAGAAGAATTTGAGATT
CAGGAACAGATGACCCTGCTGAATGCTAGATGGGAGGCTCTTAGG
GTGGAGAGTATGGACAGACAGTCCCGGCTGCACGATGTGCTGATG
GAACTGCAGAAGAAGCAACTGCAGCAGCTCTCCGCCTGGTTAACA
CTCACAGAGGAGCGCATTCAGAAGATGGAAACTTGCCCCCTGGAT
GATGATGTAAAATCTCTACAAAAGCTGCTAGAAGAACATAAAAGT
TTGCAAAGTGATCTTGAGGCTGAACAGGTGAAAGTAAATTCACTA
ACTCACATGGTGGTCATTGTTGATGAAAACAGTGGTGAGAGTGCT
ACAGCTATCCTAGAAGACCAGTTACAGAAACTTGGTGAGCGCTGG
ACAGCAGTATGCCGTTGGACTGAAGAACGCTGGAATAGGTTACAA
GAAATCGCCGCTAGCGCCCGATTTGACAGATCTGTTGAGAAATGG
CGGCGTTTTCATTATGATATAAAGATATTTAATCAGTGGCTAACA
GAAGCTGAACAGTTTCTCAGAAAGACACAAATTCCTGAGAATTGG
GAACATGCTAAATACAAATGGTATCTTAAGGAACTCCAGGATGGC
ATTGGGCAGCGGCAAACTGTTGTCAGAACATTGAATGCAACTGGG
GAAGAAATAATTCAGCAATCCTCAAAAACAGATGCCAGTATTCTA
CAGGAAAAATTGGGAAGCCTGAATCTGCGGTGGCAGGAGGTCTGC
AAACAGCTGTCAGACAGAAAAAAGAGGCTAGAAGAACAAAAGCAG
CCTGACCTAGCTCCTGGACTGACCACTATTGGAGCCTCTCCTACT
CAGACTGTTACTCTGGTGACACAACCTGTGGTTACTAAGGAAACT
GCCATCTCCAAACTAGAAATGCCATCTTCCTTGATGTTGGAGGTA
CCTGAAGAAACTCATAGATTACTGCAACAGTTCCCCCTGGACCTG
GAAAAGTTTCTTGCCTGGCTTACAGAAGCTGAAACAACTGCCAAT
GTCCTACAGGATGCTACCCGTAAGGAAAGGCTCCTAGAAGACTCC
AAGGGAGTAAAAGAGCTGATGAAACAATGGCAAGACCTCCAAGGT
GAAATTGAAGCTCACACAGATGTTTATCACAACCTGGATGAAAAC
AGCCAAAAAATCCTGAGATCCCTGGAAGGTTCCGATGATGCAGTC
CTGTTACAAAGACGTTTGGATAACATGAACTTCAAGTGGAGTGAA
CTTCGGAAAAAGTCTCTCAACATTAGGTCCCATTTGGAAGCCAGT
TCTGACCAGTGGAAGCGTCTGGGTCCAGCATCTCAGCACTTTCTT
TCCACGTCTGTCCAGGGTCCCTGGGAGAGAGCCATCTCGCCAAAC
AAAGTGCCCTACTATATCAACCACGAGACTCAAACAACTTGCTGG
GACCATCCCAAAATGACAGAGCTCTACCAGTCTTTAGCTGACCTG
AATAATGTCAGATTCTCAGCTTATAGGACTGCCATGAAACTCCGA
AGACTGCAGAAGGCCCTTTGCTTGGATCTCTTGAGCCTGTCAGCT
GCATGTGATGCCTTGGACCAGCACAACCTCAAGCAAAATGACCAG
CCCATGGATATCCTGCAGATTATTAATTGTTTGACCACTATTTAT
GACCGCCTGGAGCAAGAGCACAACAATTTGGTCAACGTCCCTCTC
TGCGTGGATATGTGTCTGAACTGGCTGCTGAATGTTTATGATACG
GGACGAACAGGGAGGATCCGTGTCCTGTCTTTTAAAACTGGCATC
ATTTCCCTGTGTAAAGCACATTTGGAAGACAAGTACAGATACCTT
TTCAAGCAAGTGGCAAGTTCAACAGGATTTTGTGACCAGCGCAGG
CTGGGCCTCCTTCTGCATGATTCTATCCAAATTCCAAGACAGTTG
GGTGAAGTTGCATCCTTTGGGGGCAGTAACATTGAGCCAAGTGTC
CGGAGCTGCTTCCAATTTGCTAATAATAAGCCAGAGATCGAAGCG
GCCCTCTTCCTAGACTGGATGAGACTGGAACCCCAGTCCATGGTG
TGGCTGCCCGTCCTGCACAGAGTGGCTGCTGCAGAAACTGCCAAG
CATCAGGCCAAATGTAACATCTGCAAAGAGTGTCCAATCATTGGA
TTCAGGTACAGGAGTCTAAAGCACTTTAATTATGACATCTGCCAA
AGCTGCTTTTTTTCTGGTCGAGTTGCAAAAGGCCATAAAATGCAC
TATCCCATGGTGGAATATTGCACTCCGACTACATCAGGAGAAGAT
GTTCGAGACTTTGCCAAGGTACTAAAAAACAAATTTCGAACCAAA
AGGTATTTTGCGAAGCATCCCCGAATGGGCTACCTGCCAGTGCAG
ACTGTCTTAGAGGGGGACAACATGGAAACTCCCGTTACTCTGATC
AACTTCTGGCCAGTAGATTCTGCGCCTGCCTCGTCCCCTCAGCTT
TCACACGATGATACTCATTCACGCATTGAACATTATGCTAGCAGG
CTAGCAGAAATGGAAAACAGCAATGGATCTTATCTAAATGATAGC
ATCTCTCCTAATGAGAGCATAGATGATGAACATTTGTTAATCCAG
CATTACTGCCAAAGTTTGAACCAGGACTCCCCCCTGAGCCAGCCT
CGTAGTCCTGCCCAGATCTTGATTTCCTTAGAGAGTGAGGAAAGA
GGGGAGCTAGAGAGAATCCTAGCAGATCTTGAGGAAGAAAACAGG
AATCTGCAAGCAGAATATGACCGTCTAAAGCAGCAGCACGAACAT
AAAGGCCTGTCCCCACTGCCGTCCCCTCCTGAAATGATGCCCACC
TCTCCCCAGAGTCCC
Human sequence with codon optimization:
(SEQ ID NO: 181)
ATGGCCAAGTACGGCGAGCACGAGGCCAGCCCCGACAACGGCCAG
AACGAGTTCAGCGACATCATCAAGAGCCGGAGCGACGAGCACAAC
GACGTGCAGAAGAAGACCTTCACCAAGTGGATCAACGCCCGGTTC
AGCAAGAGCGGCAAGCCCCCCATCAACGACATGTTCACCGACCTG
AAGGACGGCCGGAAGCTGCTGGACCTGCTGGAGGGCCTGACCGGC
ACCAGCCTGCCCAAGGAGCGGGGCAGCACCCGGGTGCACGCCCTG
AACAACGTGAACCGGGTGCTGCAGGTGCTGCACCAGAACAACGTG
GAGCTGGTGAACATCGGCGGCACCGACATCGTGGACGGCAACCAC
AAGCTGACCCTGGGCCTGCTGTGGAGCATCATCCTGCACTGGCAG
GTGAAGGACGTGATGAAGGACGTGATGAGCGACCTGCAGCAGACC
AACAGCGAGAAGATCCTGCTGAGCTGGGTGCGGCAGACCACCCGG
CCCTACAGCCAGGTGAACGTGCTGAACTTCACCACCAGCTGGACC
GACGGCCTGGCCTTCAACGCCGTGCTGCACCGGCACAAGCCCGAC
CTGTTCAGCTGGGACAAGGTGGTGAAGATGAGCCCCATCGAGCGG
CTGGAGCACGCCTTCAGCAAGGCCCAGACCTACCTGGGCATCGAG
AAGCTGCTGGACCCCGAGGACGTGGCCGTGCAGCTGCCCGACAAG
AAGAGCATCATCATGTACCTGACCAGCCTGTTCGAGGTGCTGCCC
CAGCAGGTGACCATCGACGCCATCCGGGAGGTGGAGACCCTGCCC
CGGAAGTACAAGAAGGAGTGCGAGGAGGAGGCCATCAACATCCAG
AGCACCGCCCCCGAGGAGGAGCACGAGAGCCCCCGGGCCGAGACC
CCCAGCACCGTGACCGAGGTGGACATGGACCTGGACAGCTACCAG
ATCGCCCTGGAGGAGGTGCTGACCTGGCTGCTGAGCGCCGAGGAC
ACCTTCCAGGAGCAGGACGACATCAGCGACGACGTGGAGGAGGTG
AAGGACCAGTTCGCCACCCACGAGGCCTTCATGATGGAGCTGACC
GCCCACCAGAGCAGCGTGGGCAGCGTGCTGCAGGCCGGCAACCAG
CTGATCACCCAGGGCACCCTGAGCGACGAGGAGGAGTTCGAGATC
CAGGAGCAGATGACCCTGCTGAACGCCCGGTGGGAGGCCCTGCGG
GTGGAGAGCATGGACCGGCAGAGCCGGCTGCACGACGTGCTGATG
GAGCTGCAGAAGAAGCAGCTGCAGCAGCTGAGCGCCTGGCTGACC
CTGACCGAGGAGCGGATCCAGAAGATGGAGACCTGCCCCCTGGAC
GACGACGTGAAGAGCCTGCAGAAGCTGCTGGAGGAGCACAAGAGC
CTGCAGAGCGACCTGGAGGCCGAGCAGGTGAAGGTGAACAGCCTG
ACCCACATGGTGGTGATCGTGGACGAGAACAGCGGCGAGAGCGCC
ACCGCCATCCTGGAGGACCAGCTGCAGAAGCTGGGCGAGCGGTGG
ACCGCCGTGTGCCGGTGGACCGAGGAGCGGTGGAACCGGCTGCAG
GAGATCGCCGCTAGCGCCCGGTTCGACCGGAGCGTGGAGAAGTGG
CGGCGGTTCCACTACGACATCAAGATCTTCAACCAGTGGCTGACC
GAGGCCGAGCAGTTCCTGCGGAAGACCCAGATCCCCGAGAACTGG
GAGCACGCCAAGTACAAGTGGTACCTGAAGGAGCTGCAGGACGGC
ATCGGCCAGCGGCAGACCGTGGTGCGGACCCTGAACGCCACCGGC
GAGGAGATCATCCAGCAGAGCAGCAAGACCGACGCCAGCATCCTG
CAGGAGAAGCTGGGCAGCCTGAACCTGCGGTGGCAGGAGGTGTGC
AAGCAGCTGAGCGACCGGAAGAAGCGGCTGGAGGAGCAGAAGCAG
CCCGACCTGGCCCCCGGCCTGACCACCATCGGCGCCAGCCCCACC
CAGACCGTGACCCTGGTGACCCAGCCCGTGGTGACCAAGGAGACC
GCCATCAGCAAGCTGGAGATGCCCAGCAGCCTGATGCTGGAGGTG
CCCGAGGAGACCCACCGGCTGCTGCAGCAGTTCCCCCTGGACCTG
GAGAAGTTCCTGGCCTGGCTGACCGAGGCCGAGACCACCGCCAAC
GTGCTGCAGGACGCCACCCGGAAGGAGCGGCTGCTGGAGGACAGC
AAGGGCGTGAAGGAGCTGATGAAGCAGTGGCAGGACCTGCAGGGC
GAGATCGAGGCCCACACCGACGTGTACCACAACCTGGACGAGAAC
AGCCAGAAGATCCTGCGGAGCCTGGAGGGCAGCGACGACGCCGTG
CTGCTGCAGCGGCGGCTGGACAACATGAACTTCAAGTGGAGCGAG
CTGCGGAAGAAGAGCCTGAACATCCGGAGCCACCTGGAGGCCAGC
AGCGACCAGTGGAAGCGGCTGGGCCCCGCCAGCCAGCACTTCCTG
AGCACCAGCGTGCAGGGCCCCTGGGAGCGGGCCATCAGCCCCAAC
AAGGTGCCCTACTACATCAACCACGAGACCCAGACCACCTGCTGG
GACCACCCCAAGATGACCGAGCTGTACCAGAGCCTGGCCGACCTG
AACAACGTGCGGTTCAGCGCCTACCGGACCGCCATGAAGCTGCGG
CGGCTGCAGAAGGCCCTGTGCCTGGACCTGCTGAGCCTGAGCGCC
GCCTGCGACGCCCTGGACCAGCACAACCTGAAGCAGAACGACCAG
CCCATGGACATCCTGCAGATCATCAACTGCCTGACCACCATCTAC
GACCGGCTGGAGCAGGAGCACAACAACCTGGTGAACGTGCCCCTG
TGCGTGGACATGTGCCTGAACTGGCTGCTGAACGTGTACGACACC
GGCCGGACCGGCCGGATCCGGGTGCTGAGCTTCAAGACCGGCATC
ATCAGCCTGTGCAAGGCCCACCTGGAGGACAAGTACCGGTACCTG
TTCAAGCAGGTGGCCAGCAGCACCGGCTTCTGCGACCAGCGGCGG
CTGGGCCTGCTGCTGCACGACAGCATCCAGATCCCCCGGCAGCTG
GGCGAGGTGGCCAGCTTCGGCGGCAGCAACATCGAGCCCAGCGTG
CGGAGCTGCTTCCAGTTCGCCAACAACAAGCCCGAGATCGAGGCC
GCCCTGTTCCTGGACTGGATGCGGCTGGAGCCCCAGAGCATGGTG
TGGCTGCCCGTGCTGCACCGGGTGGCCGCCGCCGAGACCGCCAAG
CACCAGGCCAAGTGCAACATCTGCAAGGAGTGCCCCATCATCGGC
TTCCGGTACCGGAGCCTGAAGCACTTCAACTACGACATCTGCCAG
AGCTGCTTCTTCAGCGGCCGGGTGGCCAAGGGCCACAAGATGCAC
TACCCCATGGTGGAGTACTGCACCCCCACCACCAGCGGCGAGGAC
GTGCGGGACTTCGCCAAGGTGCTGAAGAACAAGTTCCGGACCAAG
CGGTACTTCGCCAAGCACCCCCGGATGGGCTACCTGCCCGTGCAG
ACCGTGCTGGAGGGCGACAACATGGAGACCCCCGTGACCCTGATC
AACTTCTGGCCCGTGGACAGCGCCCCCGCCAGCAGCCCCCAGCTG
AGCCACGACGACACCCACAGCCGGATCGAGCACTACGCCAGCCGG
CTGGCCGAGATGGAGAACAGCAACGGCAGCTACCTGAACGACAGC
ATCAGCCCCAACGAGAGCATCGACGACGAGCACCTGCTGATCCAG
CACTACTGCCAGAGCCTGAACCAGGACAGCCCCCTGAGCCAGCCC
CGGAGCCCCGCCCAGATCCTGATCAGCCTGGAGAGCGAGGAGCGG
GGCGAGCTGGAGCGGATCCTGGCCGACCTGGAGGAGGAGAACCGG
AACCTGCAGGCCGAGTACGACCGGCTGAAGCAGCAGCACGAGCAC
AAGGGCCTGAGCCCCCTGCCCAGCCCCCCCGAGATGATGCCCACC
AGCCCCCAGAGCCCC
UtrophinNterm-R1_DystrophinR17_H3_R21-
R22_H4-trCterm + SynBS + coiled coil
Human sequence without codon alterations:
(SEQ ID NO: 182)
ATGGCCAAGTATGGAGAACATGAAGCCAGTCCTGACAATGGGCAG
AACGAATTCAGTGATATCATTAAGTCCAGATCTGATGAACACAAT
GACGTACAGAAGAAAACCTTTACCAAATGGATAAATGCTCGATTT
TCAAAGAGTGGGAAACCACCCATCAATGATATGTTCACAGACCTC
AAAGATGGAAGGAAGCTATTGGATCTTCTAGAAGGCCTCACAGGA
ACATCACTGCCAAAGGAACGTGGTTCCACAAGGGTACATGCCTTA
AATAACGTCAACAGAGTGCTGCAGGTTTTACATCAGAACAATGTG
GAATTAGTGAATATAGGGGGAACTGACATTGTGGATGGAAATCAC
AAACTGACTTTGGGGTTACTTTGGAGCATCATTTTGCACTGGCAG
GTGAAAGATGTCATGAAGGATGTCATGTCGGACCTGCAGCAGACG
AACAGTGAGAAGATCCTGCTCAGCTGGGTGCGTCAGACCACCAGG
CCCTACAGCCAAGTCAACGTCCTCAACTTCACCACCAGCTGGACA
GATGGACTCGCCTTTAATGCTGTCCTCCACCGACATAAACCTGAT
CTCTTCAGCTGGGATAAAGTTGTCAAAATGTCACCAATTGAGAGA
CTTGAACATGCCTTCAGCAAGGCTCAAACTTATTTGGGAATTGAA
AAGCTGTTAGATCCTGAAGATGTTGCCGTTCAGCTTCCTGACAAG
AAATCCATAATTATGTATTTAACATCTTTGTTTGAGGTGCTACCT
CAGCAAGTCACCATAGACGCCATCCGTGAGGTAGAGACACTCCCA
AGGAAATATAAAAAAGAATGTGAAGAAGAGGCAATTAATATACAG
AGTACAGCGCCTGAGGAGGAGCATGAGAGTCCCCGAGCTGAAACT
CCCAGCACTGTCACTGAGGTTGACATGGATCTGGACAGCTATCAG
ATTGCGTTGGAGGAAGTGCTGACCTGGTTGCTTTCTGCTGAGGAC
ACTTTCCAGGAGCAGGATGATATTTCTGATGATGTTGAAGAAGTC
AAAGACCAGTTTGCAACCCATGAAGCTTTTATGATGGAACTGACT
GCACACCAGAGCAGTGTGGGCAGCGTCCTGCAGGCAGGCAACCAA
CTGATAACACAAGGAACTCTGTCAGACGAAGAAGAATTTGAGATT
CAGGAACAGATGACCCTGCTGAATGCTAGATGGGAGGCTCTTAGG
GTGGAGAGTATGGACAGACAGTCCCGGCTGCACGATGTGCTGATG
GCCGCTAGCGCCCGATTTGACAGATCTGTTGAGAAATGGCGGCGT
TTTCATTATGATATAAAGATATTTAATCAGTGGCTAACAGAAGCT
GAACAGTTTCTCAGAAAGACACAAATTCCTGAGAATTGGGAACAT
GCTAAATACAAATGGTATCTTAAGGAACTCCAGGATGGCATTGGG
CAGCGGCAAACTGTTGTCAGAACATTGAATGCAACTGGGGAAGAA
ATAATTCAGCAATCCTCAAAAACAGATGCCAGTATTCTACAGGAA
AAATTGGGAAGCCTGAATCTGCGGTGGCAGGAGGTCTGCAAACAG
CTGTCAGACAGAAAAAAGAGGCTAGAAGAACAAAAGCAGCCTGAC
CTAGCTCCTGGACTGACCACTATTGGAGCCTCTCCTACTCAGACT
GTTACTCTGGTGACACAACCTGTGGTTACTAAGGAAACTGCCATC
TCCAAACTAGAAATGCCATCTTCCTTGATGTTGGAGGTACCTAAG
GATTCAACACAATGGCTGGAAGCTAAGGAAGAAGCTGAGCAGGTC
TTAGGACAGGCCAGAGCCAAGCTTGAGTCATGGAAGGAGGGTCCC
TATACAGTAGATGCAATCCAAAAGAAAATCACAGAAACCAAGCAG
TTGGCCAAAGACCTCCGCCAGTGGCAGACAAATGTAGATGTGGCA
AATGACTTGGCCCTGAAACTTCTCCGGGATTATTCTGCAGATGAT
ACCAGAAAAGTCCACATGATAACAGAGAATATCAATGCCTCTTGG
AGAAGCATTCATAAAAGGGTGAGTGAGCGAGAGGCTGCTTTGGAA
GAAACTCATAGATTACTGCAACAGTTCCCCCTGGACCTGGAAAAG
TTTCTTGCCTGGCTTACAGAAGCTGAAACAACTGCCAATGTCCTA
CAGGATGCTACCCGTAAGGAAAGGCTCCTAGAAGACTCCAAGGGA
GTAAAAGAGCTGATGAAACAATGGCAAGACCTCCAAGGTGAAATT
GAAGCTCACACAGATGTTTATCACAACCTGGATGAAAACAGCCAA
AAAATCCTGAGATCCCTGGAAGGTTCCGATGATGCAGTCCTGTTA
CAAAGACGTTTGGATAACATGAACTTCAAGTGGAGTGAACTTCGG
AAAAAGTCTCTCAACATTAGGTCCCATTTGGAAGCCAGTTCTGAC
CAGTGGAAGCGTCTGGGTCCAGCATCTCAGCACTTTCTTTCCACG
TCTGTCCAGGGTCCCTGGGAGAGAGCCATCTCGCCAAACAAAGTG
CCCTACTATATCAACCACGAGACTCAAACAACTTGCTGGGACCAT
CCCAAAATGACAGAGCTCTACCAGTCTTTAGCTGACCTGAATAAT
GTCAGATTCTCAGCTTATAGGACTGCCATGAAACTCCGAAGACTG
CAGAAGGCCCTTTGCTTGGATCTCTTGAGCCTGTCAGCTGCATGT
GATGCCTTGGACCAGCACAACCTCAAGCAAAATGACCAGCCCATG
GATATCCTGCAGATTATTAATTGTTTGACCACTATTTATGACCGC
CTGGAGCAAGAGCACAACAATTTGGTCAACGTCCCTCTCTGCGTG
GATATGTGTCTGAACTGGCTGCTGAATGTTTATGATACGGGACGA
ACAGGGAGGATCCGTGTCCTGTCTTTTAAAACTGGCATCATTTCC
CTGTGTAAAGCACATTTGGAAGACAAGTACAGATACCTTTTCAAG
CAAGTGGCAAGTTCAACAGGATTTTGTGACCAGCGCAGGCTGGGC
CTCCTTCTGCATGATTCTATCCAAATTCCAAGACAGTTGGGTGAA
GTTGCATCCTTTGGGGGCAGTAACATTGAGCCAAGTGTCCGGAGC
TGCTTCCAATTTGCTAATAATAAGCCAGAGATCGAAGCGGCCCTC
TTCCTAGACTGGATGAGACTGGAACCCCAGTCCATGGTGTGGCTG
CCCGTCCTGCACAGAGTGGCTGCTGCAGAAACTGCCAAGCATCAG
GCCAAATGTAACATCTGCAAAGAGTGTCCAATCATTGGATTCAGG
TACAGGAGTCTAAAGCACTTTAATTATGACATCTGCCAAAGCTGC
TTTTTTTCTGGTCGAGTTGCAAAAGGCCATAAAATGCACTATCCC
ATGGTGGAATATTGCACTCCGACTACATCAGGAGAAGATGTTCGA
GACTTTGCCAAGGTACTAAAAAACAAATTTCGAACCAAAAGGTAT
TTTGCGAAGCATCCCCGAATGGGCTACCTGCCAGTGCAGACTGTC
TTAGAGGGGGACAACATGGAAACTCCCGTTACTCTGATCAACTTC
TGGCCAGTAGATTCTGCGCCTGCCTCGTCCCCTCAGCTTTCACAC
GATGATACTCATTCACGCATTGAACATTATGCTAGCAGGCTAGCA
GAAATGGAAAACAGCAATGGATCTTATCTAAATGATAGCATCTCT
CCTAATGAGAGCATAGATGATGAACATTTGTTAATCCAGCATTAC
TGCCAAAGTTTGAACCAGGACTCCCCCCTGAGCCAGCCTCGTAGT
CCTGCCCAGATCTTGATTTCCTTAGAGAGTGAGGAAAGAGGGGAG
CTAGAGAGAATCCTAGCAGATCTTGAGGAAGAAAACAGGAATCTG
CAAGCAGAATATGACCGTCTAAAGCAGCAGCACGAACATAAAGGC
CTGTCCCCACTGCCGTCCCCTCCTGAAATGATGCCCACCTCTCCC
CAGAGTCCC
Human sequence with codon optimization:
(SEQ ID NO: 183)
ATGGCCAAGTACGGCGAGCACGAGGCCAGCCCCGACAACGGCCAG
AACGAGTTCAGCGACATCATCAAGAGCCGGAGCGACGAGCACAAC
GACGTGCAGAAGAAGACCTTCACCAAGTGGATCAACGCCCGGTTC
AGCAAGAGCGGCAAGCCCCCCATCAACGACATGTTCACCGACCTG
AAGGACGGCCGGAAGCTGCTGGACCTGCTGGAGGGCCTGACCGGC
ACCAGCCTGCCCAAGGAGCGGGGCAGCACCCGGGTGCACGCCCTG
AACAACGTGAACCGGGTGCTGCAGGTGCTGCACCAGAACAACGTG
GAGCTGGTGAACATCGGCGGCACCGACATCGTGGACGGCAACCAC
AAGCTGACCCTGGGCCTGCTGTGGAGCATCATCCTGCACTGGCAG
GTGAAGGACGTGATGAAGGACGTGATGAGCGACCTGCAGCAGACC
AACAGCGAGAAGATCCTGCTGAGCTGGGTGCGGCAGACCACCCGG
CCCTACAGCCAGGTGAACGTGCTGAACTTCACCACCAGCTGGACC
GACGGCCTGGCCTTCAACGCCGTGCTGCACCGGCACAAGCCCGAC
CTGTTCAGCTGGGACAAGGTGGTGAAGATGAGCCCCATCGAGCGG
CTGGAGCACGCCTTCAGCAAGGCCCAGACCTACCTGGGCATCGAG
AAGCTGCTGGACCCCGAGGACGTGGCCGTGCAGCTGCCCGACAAG
AAGAGCATCATCATGTACCTGACCAGCCTGTTCGAGGTGCTGCCC
CAGCAGGTGACCATCGACGCCATCCGGGAGGTGGAGACCCTGCCC
CGGAAGTACAAGAAGGAGTGCGAGGAGGAGGCCATCAACATCCAG
AGCACCGCCCCCGAGGAGGAGCACGAGAGCCCCCGGGCCGAGACC
CCCAGCACCGTGACCGAGGTGGACATGGACCTGGACAGCTACCAG
ATCGCCCTGGAGGAGGTGCTGACCTGGCTGCTGAGCGCCGAGGAC
ACCTTCCAGGAGCAGGACGACATCAGCGACGACGTGGAGGAGGTG
AAGGACCAGTTCGCCACCCACGAGGCCTTCATGATGGAGCTGACC
GCCCACCAGAGCAGCGTGGGCAGCGTGCTGCAGGCCGGCAACCAG
CTGATCACCCAGGGCACCCTGAGCGACGAGGAGGAGTTCGAGATC
CAGGAGCAGATGACCCTGCTGAACGCCCGGTGGGAGGCCCTGCGG
GTGGAGAGCATGGACCGGCAGAGCCGGCTGCACGACGTGCTGATG
GAGCTGCAGAAGAAGCAGCTGCAGCAGCTGAGCGCCTGGCTGACC
CTGACCGAGGAGCGGATCCAGAAGATGGAGACCTGCCCCCTGGAC
GACGACGTGAAGAGCCTGCAGAAGCTGCTGGAGGAGCACAAGAGC
CTGCAGAGCGACCTGGAGGCCGAGCAGGTGAAGGTGAACAGCCTG
ACCCACATGGTGGTGATCGTGGACGAGAACAGCGGCGAGAGCGCC
ACCGCCATCCTGGAGGACCAGCTGCAGAAGCTGGGCGAGCGGTGG
ACCGCCGTGTGCCGGTGGACCGAGGAGCGGTGGAACCGGCTGCAG
GAGATCGCCGCTAGCGCCCGGTTCGACCGGAGCGTGGAGAAGTGG
CGGCGGTTCCACTACGACATCAAGATCTTCAACCAGTGGCTGACC
GAGGCCGAGCAGTTCCTGCGGAAGACCCAGATCCCCGAGAACTGG
GAGCACGCCAAGTACAAGTGGTACCTGAAGGAGCTGCAGGACGGC
ATCGGCCAGCGGCAGACCGTGGTGCGGACCCTGAACGCCACCGGC
GAGGAGATCATCCAGCAGAGCAGCAAGACCGACGCCAGCATCCTG
CAGGAGAAGCTGGGCAGCCTGAACCTGCGGTGGCAGGAGGTGTGC
AAGCAGCTGAGCGACCGGAAGAAGCGGCTGGAGGAGCAGAAGCAG
CCCGACCTGGCCCCCGGCCTGACCACCATCGGCGCCAGCCCCACC
CAGACCGTGACCCTGGTGACCCAGCCCGTGGTGACCAAGGAGACC
GCCATCAGCAAGCTGGAGATGCCCAGCAGCCTGATGCTGGAGGTG
CCCAAGGACAGCACCCAGTGGCTGGAGGCCAAGGAGGAGGCCGAG
CAGGTGCTGGGCCAGGCCCGGGCCAAGCTGGAGAGCTGGAAGGAG
GGCCCCTACACCGTGGACGCCATCCAGAAGAAGATCACCGAGACC
AAGCAGCTGGCCAAGGACCTGCGGCAGTGGCAGACCAACGTGGAC
GTGGCCAACGACCTGGCCCTGAAGCTGCTGCGGGACTACAGCGCC
GACGACACCCGGAAGGTGCACATGATCACCGAGAACATCAACGCC
AGCTGGCGGAGCATCCACAAGCGGGTGAGCGAGCGGGAGGCCGCC
CTGGAGGAGACCCACCGGCTGCTGCAGCAGTTCCCCCTGGACCTG
GAGAAGTTCCTGGCCTGGCTGACCGAGGCCGAGACCACCGCCAAC
GTGCTGCAGGACGCCACCCGGAAGGAGCGGCTGCTGGAGGACAGC
AAGGGCGTGAAGGAGCTGATGAAGCAGTGGCAGGACCTGCAGGGC
GAGATCGAGGCCCACACCGACGTGTACCACAACCTGGACGAGAAC
AGCCAGAAGATCCTGCGGAGCCTGGAGGGCAGCGACGACGCCGTG
CTGCTGCAGCGGCGGCTGGACAACATGAACTTCAAGTGGAGCGAG
CTGCGGAAGAAGAGCCTGAACATCCGGAGCCACCTGGAGGCCAGC
AGCGACCAGTGGAAGCGGCTGGGCCCCGCCAGCCAGCACTTCCTG
AGCACCAGCGTGCAGGGCCCCTGGGAGCGGGCCATCAGCCCCAAC
AAGGTGCCCTACTACATCAACCACGAGACCCAGACCACCTGCTGG
GACCACCCCAAGATGACCGAGCTGTACCAGAGCCTGGCCGACCTG
AACAACGTGCGGTTCAGCGCCTACCGGACCGCCATGAAGCTGCGG
CGGCTGCAGAAGGCCCTGTGCCTGGACCTGCTGAGCCTGAGCGCC
GCCTGCGACGCCCTGGACCAGCACAACCTGAAGCAGAACGACCAG
CCCATGGACATCCTGCAGATCATCAACTGCCTGACCACCATCTAC
GACCGGCTGGAGCAGGAGCACAACAACCTGGTGAACGTGCCCCTG
TGCGTGGACATGTGCCTGAACTGGCTGCTGAACGTGTACGACACC
GGCCGGACCGGCCGGATCCGGGTGCTGAGCTTCAAGACCGGCATC
ATCAGCCTGTGCAAGGCCCACCTGGAGGACAAGTACCGGTACCTG
TTCAAGCAGGTGGCCAGCAGCACCGGCTTCTGCGACCAGCGGCGG
CTGGGCCTGCTGCTGCACGACAGCATCCAGATCCCCCGGCAGCTG
GGCGAGGTGGCCAGCTTCGGCGGCAGCAACATCGAGCCCAGCGTG
CGGAGCTGCTTCCAGTTCGCCAACAACAAGCCCGAGATCGAGGCC
GCCCTGTTCCTGGACTGGATGCGGCTGGAGCCCCAGAGCATGGTG
TGGCTGCCCGTGCTGCACCGGGTGGCCGCCGCCGAGACCGCCAAG
CACCAGGCCAAGTGCAACATCTGCAAGGAGTGCCCCATCATCGGC
TTCCGGTACCGGAGCCTGAAGCACTTCAACTACGACATCTGCCAG
AGCTGCTTCTTCAGCGGCCGGGTGGCCAAGGGCCACAAGATGCAC
TACCCCATGGTGGAGTACTGCACCCCCACCACCAGCGGCGAGGAC
GTGCGGGACTTCGCCAAGGTGCTGAAGAACAAGTTCCGGACCAAG
CGGTACTTCGCCAAGCACCCCCGGATGGGCTACCTGCCCGTGCAG
ACCGTGCTGGAGGGCGACAACATGGAGACCCCCGTGACCCTGATC
AACTTCTGGCCCGTGGACAGCGCCCCCGCCAGCAGCCCCCAGCTG
AGCCACGACGACACCCACAGCCGGATCGAGCACTACGCCAGCCGG
CTGGCCGAGATGGAGAACAGCAACGGCAGCTACCTGAACGACAGC
ATCAGCCCCAACGAGAGCATCGACGACGAGCACCTGCTGATCCAG
CACTACTGCCAGAGCCTGAACCAGGACAGCCCCCTGAGCCAGCCC
CGGAGCCCCGCCCAGATCCTGATCAGCCTGGAGAGCGAGGAGCGG
GGCGAGCTGGAGCGGATCCTGGCCGACCTGGAGGAGGAGAACCGG
AACCTGCAGGCCGAGTACGACCGGCTGAAGCAGCAGCACGAGCAC
AAGGGCCTGAGCCCCCTGCCCAGCCCCCCCGAGATGATGCCCACC
AGCCCCCAGAGCCCC
UtrophinNterm-R2_DystrophinR17_R21-R22_
H4-trCterm + SynBS
Human sequence without codon alterations:
(SEQ ID NO: 184)
ATGGCCAAGTATGGAGAACATGAAGCCAGTCCTGACAATGGGCAG
AACGAATTCAGTGATATCATTAAGTCCAGATCTGATGAACACAAT
GACGTACAGAAGAAAACCTTTACCAAATGGATAAATGCTCGATTT
TCAAAGAGTGGGAAACCACCCATCAATGATATGTTCACAGACCTC
AAAGATGGAAGGAAGCTATTGGATCTTCTAGAAGGCCTCACAGGA
ACATCACTGCCAAAGGAACGTGGTTCCACAAGGGTACATGCCTTA
AATAACGTCAACAGAGTGCTGCAGGTTTTACATCAGAACAATGTG
GAATTAGTGAATATAGGGGGAACTGACATTGTGGATGGAAATCAC
AAACTGACTTTGGGGTTACTTTGGAGCATCATTTTGCACTGGCAG
GTGAAAGATGTCATGAAGGATGTCATGTCGGACCTGCAGCAGACG
AACAGTGAGAAGATCCTGCTCAGCTGGGTGCGTCAGACCACCAGG
CCCTACAGCCAAGTCAACGTCCTCAACTTCACCACCAGCTGGACA
GATGGACTCGCCTTTAATGCTGTCCTCCACCGACATAAACCTGAT
CTCTTCAGCTGGGATAAAGTTGTCAAAATGTCACCAATTGAGAGA
CTTGAACATGCCTTCAGCAAGGCTCAAACTTATTTGGGAATTGAA
AAGCTGTTAGATCCTGAAGATGTTGCCGTTCAGCTTCCTGACAAG
AAATCCATAATTATGTATTTAACATCTTTGTTTGAGGTGCTACCT
CAGCAAGTCACCATAGACGCCATCCGTGAGGTAGAGACACTCCCA
AGGAAATATAAAAAAGAATGTGAAGAAGAGGCAATTAATATACAG
AGTACAGCGCCTGAGGAGGAGCATGAGAGTCCCCGAGCTGAAACT
CCCAGCACTGTCACTGAGGTTGACATGGATCTGGACAGCTATCAG
ATTGCGTTGGAGGAAGTGCTGACCTGGTTGCTTTCTGCTGAGGAC
ACTTTCCAGGAGCAGGATGATATTTCTGATGATGTTGAAGAAGTC
AAAGACCAGTTTGCAACCCATGAAGCTTTTATGATGGAACTGACT
GCACACCAGAGCAGTGTGGGCAGCGTCCTGCAGGCAGGCAACCAA
CTGATAACACAAGGAACTCTGTCAGACGAAGAAGAATTTGAGATT
CAGGAACAGATGACCCTGCTGAATGCTAGATGGGAGGCTCTTAGG
GTGGAGAGTATGGACAGACAGTCCCGGCTGCACGATGTGCTGATG
GAACTGCAGAAGAAGCAACTGCAGCAGCTCTCCGCCTGGTTAACA
CTCACAGAGGAGCGCATTCAGAAGATGGAAACTTGCCCCCTGGAT
GATGATGTAAAATCTCTACAAAAGCTGCTAGAAGAACATAAAAGT
TTGCAAAGTGATCTTGAGGCTGAACAGGTGAAAGTAAATTCACTA
ACTCACATGGTGGTCATTGTTGATGAAAACAGTGGTGAGAGTGCT
ACAGCTATCCTAGAAGACCAGTTACAGAAACTTGGTGAGCGCTGG
ACAGCAGTATGCCGTTGGACTGAAGAACGCTGGAATAGGTTACAA
GAAATCGCCGCTAGCGCCCGATTTGACAGATCTGTTGAGAAATGG
CGGCGTTTTCATTATGATATAAAGATATTTAATCAGTGGCTAACA
GAAGCTGAACAGTTTCTCAGAAAGACACAAATTCCTGAGAATTGG
GAACATGCTAAATACAAATGGTATCTTAAGGAACTCCAGGATGGC
ATTGGGCAGCGGCAAACTGTTGTCAGAACATTGAATGCAACTGGG
GAAGAAATAATTCAGCAATCCTCAAAAACAGATGCCAGTATTCTA
CAGGAAAAATTGGGAAGCCTGAATCTGCGGTGGCAGGAGGTCTGC
AAACAGCTGTCAGACAGAAAAAAGAGGCTAGAAGAACAAAAGAAG
GATTCAACACAATGGCTGGAAGCTAAGGAAGAAGCTGAGCAGGTC
TTAGGACAGGCCAGAGCCAAGCTTGAGTCATGGAAGGAGGGTCCC
TATACAGTAGATGCAATCCAAAAGAAAATCACAGAAACCAAGCAG
TTGGCCAAAGACCTCCGCCAGTGGCAGACAAATGTAGATGTGGCA
AATGACTTGGCCCTGAAACTTCTCCGGGATTATTCTGCAGATGAT
ACCAGAAAAGTCCACATGATAACAGAGAATATCAATGCCTCTTGG
AGAAGCATTCATAAAAGGGTGAGTGAGCGAGAGGCTGCTTTGGAA
GAAACTCATAGATTACTGCAACAGTTCCCCCTGGACCTGGAAAAG
TTTCTTGCCTGGCTTACAGAAGCTGAAACAACTGCCAATGTCCTA
CAGGATGCTACCCGTAAGGAAAGGCTCCTAGAAGACTCCAAGGGA
GTAAAAGAGCTGATGAAACAATGGCAAGACCTCCAAGGTGAAATT
GAAGCTCACACAGATGTTTATCACAACCTGGATGAAAACAGCCAA
AAAATCCTGAGATCCCTGGAAGGTTCCGATGATGCAGTCCTGTTA
CAAAGACGTTTGGATAACATGAACTTCAAGTGGAGTGAACTTCGG
AAAAAGTCTCTCAACATTAGGTCCCATTTGGAAGCCAGTTCTGAC
CAGTGGAAGCGTCTGGGTCCAGCATCTCAGCACTTTCTTTCCACG
TCTGTCCAGGGTCCCTGGGAGAGAGCCATCTCGCCAAACAAAGTG
CCCTACTATATCAACCACGAGACTCAAACAACTTGCTGGGACCAT
CCCAAAATGACAGAGCTCTACCAGTCTTTAGCTGACCTGAATAAT
GTCAGATTCTCAGCTTATAGGACTGCCATGAAACTCCGAAGACTG
CAGAAGGCCCTTTGCTTGGATCTCTTGAGCCTGTCAGCTGCATGT
GATGCCTTGGACCAGCACAACCTCAAGCAAAATGACCAGCCCATG
GATATCCTGCAGATTATTAATTGTTTGACCACTATTTATGACCGC
CTGGAGCAAGAGCACAACAATTTGGTCAACGTCCCTCTCTGCGTG
GATATGTGTCTGAACTGGCTGCTGAATGTTTATGATACGGGACGA
ACAGGGAGGATCCGTGTCCTGTCTTTTAAAACTGGCATCATTTCC
CTGTGTAAAGCACATTTGGAAGACAAGTACAGATACCTTTTCAAG
CAAGTGGCAAGTTCAACAGGATTTTGTGACCAGCGCAGGCTGGGC
CTCCTTCTGCATGATTCTATCCAAATTCCAAGACAGTTGGGTGAA
GTTGCATCCTTTGGGGGCAGTAACATTGAGCCAAGTGTCCGGAGC
TGCTTCCAATTTGCTAATAATAAGCCAGAGATCGAAGCGGCCCTC
TTCCTAGACTGGATGAGACTGGAACCCCAGTCCATGGTGTGGCTG
CCCGTCCTGCACAGAGTGGCTGCTGCAGAAACTGCCAAGCATCAG
GCCAAATGTAACATCTGCAAAGAGTGTCCAATCATTGGATTCAGG
TACAGGAGTCTAAAGCACTTTAATTATGACATCTGCCAAAGCTGC
TTTTTTTCTGGTCGAGTTGCAAAAGGCCATAAAATGCACTATCCC
ATGGTGGAATATTGCACTCCGACTACATCAGGAGAAGATGTTCGA
GACTTTGCCAAGGTACTAAAAAACAAATTTCGAACCAAAAGGTAT
TTTGCGAAGCATCCCCGAATGGGCTACCTGCCAGTGCAGACTGTC
TTAGAGGGGGACAACATGGAAACTCCCGTTACTCTGATCAACTTC
TGGCCAGTAGATTCTGCGCCTGCCTCGTCCCCTCAGCTTTCACAC
GATGATACTCATTCACGCATTGAACATTATGCTAGCAGGCTAGCA
GAAATGGAAAACAGCAATGGATCTTATCTAAATGATAGCATCTCT
CCTAATGAGAGCATAGATGATGAACATTTGTTAATCCAGCATTAC
TGCCAAAGTTTGAACCAGGACTCCCCCCTGAGCCAGCCTCGTAGT
Human sequence with codon optimization:
(SEQ ID NO: 185)
ATGGCCAAGTACGGCGAGCACGAGGCCAGCCCCGACAACGGCCAG
AACGAGTTCAGCGACATCATCAAGAGCCGGAGCGACGAGCACAAC
GACGTGCAGAAGAAGACCTTCACCAAGTGGATCAACGCCCGGTTC
AGCAAGAGCGGCAAGCCCCCCATCAACGACATGTTCACCGACCTG
AAGGACGGCCGGAAGCTGCTGGACCTGCTGGAGGGCCTGACCGGC
ACCAGCCTGCCCAAGGAGCGGGGCAGCACCCGGGTGCACGCCCTG
AACAACGTGAACCGGGTGCTGCAGGTGCTGCACCAGAACAACGTG
GAGCTGGTGAACATCGGCGGCACCGACATCGTGGACGGCAACCAC
AAGCTGACCCTGGGCCTGCTGTGGAGCATCATCCTGCACTGGCAG
GTGAAGGACGTGATGAAGGACGTGATGAGCGACCTGCAGCAGACC
AACAGCGAGAAGATCCTGCTGAGCTGGGTGCGGCAGACCACCCGG
CCCTACAGCCAGGTGAACGTGCTGAACTTCACCACCAGCTGGACC
GACGGCCTGGCCTTCAACGCCGTGCTGCACCGGCACAAGCCCGAC
CTGTTCAGCTGGGACAAGGTGGTGAAGATGAGCCCCATCGAGCGG
CTGGAGCACGCCTTCAGCAAGGCCCAGACCTACCTGGGCATCGAG
AAGCTGCTGGACCCCGAGGACGTGGCCGTGCAGCTGCCCGACAAG
AAGAGCATCATCATGTACCTGACCAGCCTGTTCGAGGTGCTGCCC
CAGCAGGTGACCATCGACGCCATCCGGGAGGTGGAGACCCTGCCC
CGGAAGTACAAGAAGGAGTGCGAGGAGGAGGCCATCAACATCCAG
AGCACCGCCCCCGAGGAGGAGCACGAGAGCCCCCGGGCCGAGACC
CCCAGCACCGTGACCGAGGTGGACATGGACCTGGACAGCTACCAG
ATCGCCCTGGAGGAGGTGCTGACCTGGCTGCTGAGCGCCGAGGAC
ACCTTCCAGGAGCAGGACGACATCAGCGACGACGTGGAGGAGGTG
AAGGACCAGTTCGCCACCCACGAGGCCTTCATGATGGAGCTGACC
GCCCACCAGAGCAGCGTGGGCAGCGTGCTGCAGGCCGGCAACCAG
CTGATCACCCAGGGCACCCTGAGCGACGAGGAGGAGTTCGAGATC
CAGGAGCAGATGACCCTGCTGAACGCCCGGTGGGAGGCCCTGCGG
GTGGAGAGCATGGACCGGCAGAGCCGGCTGCACGACGTGCTGATG
GAGCTGCAGAAGAAGCAGCTGCAGCAGCTGAGCGCCTGGCTGACC
CTGACCGAGGAGCGGATCCAGAAGATGGAGACCTGCCCCCTGGAC
GACGACGTGAAGAGCCTGCAGAAGCTGCTGGAGGAGCACAAGAGC
CTGCAGAGCGACCTGGAGGCCGAGCAGGTGAAGGTGAACAGCCTG
ACCCACATGGTGGTGATCGTGGACGAGAACAGCGGCGAGAGCGCC
ACCGCCATCCTGGAGGACCAGCTGCAGAAGCTGGGCGAGCGGTGG
ACCGCCGTGTGCCGGTGGACCGAGGAGCGGTGGAACCGGCTGCAG
GAGATCGCCGCTAGCGCCCGGTTCGACCGGAGCGTGGAGAAGTGG
CGGCGGTTCCACTACGACATCAAGATCTTCAACCAGTGGCTGACC
GAGGCCGAGCAGTTCCTGCGGAAGACCCAGATCCCCGAGAACTGG
GAGCACGCCAAGTACAAGTGGTACCTGAAGGAGCTGCAGGACGGC
ATCGGCCAGCGGCAGACCGTGGTGCGGACCCTGAACGCCACCGGC
GAGGAGATCATCCAGCAGAGCAGCAAGACCGACGCCAGCATCCTG
CAGGAGAAGCTGGGCAGCCTGAACCTGCGGTGGCAGGAGGTGTGC
AAGCAGCTGAGCGACCGGAAGAAGCGGCTGGAGGAGCAGAAGAAG
GACAGCACCCAGTGGCTGGAGGCCAAGGAGGAGGCCGAGCAGGTG
CTGGGCCAGGCCCGGGCCAAGCTGGAGAGCTGGAAGGAGGGCCCC
TACACCGTGGACGCCATCCAGAAGAAGATCACCGAGACCAAGCAG
CTGGCCAAGGACCTGCGGCAGTGGCAGACCAACGTGGACGTGGCC
AACGACCTGGCCCTGAAGCTGCTGCGGGACTACAGCGCCGACGAC
ACCCGGAAGGTGCACATGATCACCGAGAACATCAACGCCAGCTGG
CGGAGCATCCACAAGCGGGTGAGCGAGCGGGAGGCCGCCCTGGAG
GAGACCCACCGGCTGCTGCAGCAGTTCCCCCTGGACCTGGAGAAG
TTCCTGGCCTGGCTGACCGAGGCCGAGACCACCGCCAACGTGCTG
CAGGACGCCACCCGGAAGGAGCGGCTGCTGGAGGACAGCAAGGGC
GTGAAGGAGCTGATGAAGCAGTGGCAGGACCTGCAGGGCGAGATC
GAGGCCCACACCGACGTGTACCACAACCTGGACGAGAACAGCCAG
AAGATCCTGCGGAGCCTGGAGGGCAGCGACGACGCCGTGCTGCTG
CAGCGGCGGCTGGACAACATGAACTTCAAGTGGAGCGAGCTGCGG
AAGAAGAGCCTGAACATCCGGAGCCACCTGGAGGCCAGCAGCGAC
CAGTGGAAGCGGCTGGGCCCCGCCAGCCAGCACTTCCTGAGCACC
AGCGTGCAGGGCCCCTGGGAGCGGGCCATCAGCCCCAACAAGGTG
CCCTACTACATCAACCACGAGACCCAGACCACCTGCTGGGACCAC
CCCAAGATGACCGAGCTGTACCAGAGCCTGGCCGACCTGAACAAC
GTGCGGTTCAGCGCCTACCGGACCGCCATGAAGCTGCGGCGGCTG
CAGAAGGCCCTGTGCCTGGACCTGCTGAGCCTGAGCGCCGCCTGC
GACGCCCTGGACCAGCACAACCTGAAGCAGAACGACCAGCCCATG
GACATCCTGCAGATCATCAACTGCCTGACCACCATCTACGACCGG
CTGGAGCAGGAGCACAACAACCTGGTGAACGTGCCCCTGTGCGTG
GACATGTGCCTGAACTGGCTGCTGAACGTGTACGACACCGGCCGG
ACCGGCCGGATCCGGGTGCTGAGCTTCAAGACCGGCATCATCAGC
CTGTGCAAGGCCCACCTGGAGGACAAGTACCGGTACCTGTTCAAG
CAGGTGGCCAGCAGCACCGGCTTCTGCGACCAGCGGCGGCTGGGC
CTGCTGCTGCACGACAGCATCCAGATCCCCCGGCAGCTGGGCGAG
GTGGCCAGCTTCGGCGGCAGCAACATCGAGCCCAGCGTGCGGAGC
TGCTTCCAGTTCGCCAACAACAAGCCCGAGATCGAGGCCGCCCTG
TTCCTGGACTGGATGCGGCTGGAGCCCCAGAGCATGGTGTGGCTG
CCCGTGCTGCACCGGGTGGCCGCCGCCGAGACCGCCAAGCACCAG
GCCAAGTGCAACATCTGCAAGGAGTGCCCCATCATCGGCTTCCGG
TACCGGAGCCTGAAGCACTTCAACTACGACATCTGCCAGAGCTGC
TTCTTCAGCGGCCGGGTGGCCAAGGGCCACAAGATGCACTACCCC
ATGGTGGAGTACTGCACCCCCACCACCAGCGGCGAGGACGTGCGG
GACTTCGCCAAGGTGCTGAAGAACAAGTTCCGGACCAAGCGGTAC
TTCGCCAAGCACCCCCGGATGGGCTACCTGCCCGTGCAGACCGTG
CTGGAGGGCGACAACATGGAGACCCCCGTGACCCTGATCAACTTC
TGGCCCGTGGACAGCGCCCCCGCCAGCAGCCCCCAGCTGAGCCAC
GACGACACCCACAGCCGGATCGAGCACTACGCCAGCCGGCTGGCC
GAGATGGAGAACAGCAACGGCAGCTACCTGAACGACAGCATCAGC
CCCAACGAGAGCATCGACGACGAGCACCTGCTGATCCAGCACTAC
TGCCAGAGCCTGAACCAGGACAGCCCCCTGAGCCAGCCCCGGAGC

In some embodiments, the present disclosure provides proteins that are variants of any one of SEQ ID Nos. 156-165. Variant polypeptides include polypeptides that differ by one or more (e.g., 1, 2, 3, 4, 5, 6, 7, 8, 9, 10, 11, 12, 13, 14, 15, 16, 17, 18, 19, 20, or more) amino acid substitutions, additions, or deletions, and will, therefore, include amino acid sequences that differ from the amino acid sequences designated in any one of SEQ ID Nos. 156-165.

In some embodiments, the present disclosure provides proteins comprising an amino acid sequence that is at least 65%, at least 68%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID Nos. 156-165.

The “percent identity” of two amino acid sequences or nucleic acid sequences may be determined by any method known in the art. In some embodiments, the percent identity of two nucleic acid sequences is determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. BLAST nucleotide searches can be performed with the NBLAST program, score=100, wordlength-12, to obtain guide sequences homologous to a target nucleic acid. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), e.g., as an “amino acid difference” as defined herein. Alternatively, the degree of sequence identity between two amino acid sequences may be calculated using a known computer algorithm (e.g., by the local homology algorithm of Smith and Waterman (1970) Adv. Appl. Math. 2: 482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

### Recombinant Nucleic Acids

The disclosure provides recombinant nucleic acids comprising a nucleotide sequence encoding a micro-dystrophin protein of the disclosure. In some embodiments, a micro-dystrophin protein as described herein further comprises one or more utrophin protein domains. A recombinant nucleic acid is a molecule that is constructed by joining nucleic acids (e.g., isolated nucleic acids, synthetic nucleic acids or a combination thereof) from multiple sources.

In some aspects, the present disclosure provides a recombinant nucleic acid comprising a nucleotide sequence encoding a micro-dystrophin protein as described herein. In some aspects, the present disclosure provides a recombinant nucleic acid comprising a nucleotide sequence encoding a chimeric protein comprising a dystrophin region (or domain) and a utrophin region (or domain). In some embodiments, the chimeric protein comprises a carboxy-terminal (also referred to herein as “C-terminal” or “C-terminus”) region of dystrophin coupled to an amino-terminal (also referred to herein as “N-terminal” or “N-terminus”) region of utrophin. In some aspects, the present disclosure provides a recombinant nucleic acid comprising a nucleotide sequence encoding a micro-dystrophin protein comprising a utrophin N-terminus. In some embodiments, the recombinant nucleic acid comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs: 7-12, and 22-39. In some embodiments, the recombinant nucleic acid comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs: 85-92, 126-131, and 133-134. In some embodiments, the recombinant nucleic acid comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs: 135-148. In some embodiments, the recombinant nucleic acid comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs: 166-185.

In some embodiments, the recombinant nucleic acid comprises a nucleotide sequence that is at least 80% identical to the nucleotide sequence of any one of SEQ ID NOs: 7-12, and 22-39. In some embodiments, the recombinant nucleic acid comprises a nucleotide sequence having 100% identity with the nucleotide sequence of any one of SEQ ID NOs: 7-12, and 22-39. In some embodiments, the recombinant nucleic acid comprises a nucleotide sequence that is at least 80% identical to the nucleotide sequence of any one of SEQ ID NOs: 85-92, 126-131, and 133-134. In some embodiments, the recombinant nucleic acid comprises a nucleotide sequence having 100% identity with the nucleotide sequence of any one of SEQ ID NOs: 85-92, 126-131, and 133-134. In some embodiments, the recombinant nucleic acid comprises a nucleotide sequence that is at least 80% identical to the nucleotide sequence of any one of SEQ ID NOs: 135-148. In some embodiments, the recombinant nucleic acid comprises a nucleotide sequence having 100% identity with the nucleotide sequence of any one of SEQ ID NOs: 135-148. In some embodiments, the recombinant nucleic acid comprises a nucleotide sequence that is at least 80% identical to the nucleotide sequence of any one of SEQ ID NOs: 166-185. In some embodiments, the recombinant nucleic acid comprises a nucleotide sequence having 100% identity with the nucleotide sequence of any one of SEQ ID NOs: 166-185.

A recombinant nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Recombinant nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

Expression of the micro-dystrophin protein may be controlled using one or more regulatory sequences such as enhancers and promoters, operably linked to the nucleotide sequences encoding the micro-dystrophin protein.

A “promoter”, as used herein, refers to a control region of a nucleic acid at which initiation and rate of transcription of the remainder of a nucleic acid sequence are controlled. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence.

Promoters that may be used in accordance with the present disclosure may comprise any suitable promoter that can drive the expression of the nucleotide sequences encoding the micro-dystrophin proteins.

In some embodiments, a promoter is operably linked to the nucleotide sequence encoding the micro-dystrophin protein. In some embodiments, the recombinant nucleic acid comprising a nucleotide sequence encoding a micro-dystrophin protein as described herein, also comprises a nucleotide sequence encoding a region of a utrophin protein. In some embodiments, a promoter is operably linked to the nucleotide sequence encoding the region of utrophin protein.

In some embodiments, a promoter is naturally associated with dystrophin, and may be obtained by isolating the 5′ non-coding sequence upstream of the coding segment and/or exon of dystrophin. Such a promoter may be referred to as an endogenous promoter or a native promoter. In some embodiments, the promoter of any of the disclosed nucleic acid molecules or constructs is an endogenous promoter, such as a dystrophin promoter. In some embodiments, the promoter of any of the disclosed nucleic acid molecules or constructs is an exogenous promoter.

In some embodiments, the promoter is a chimeric promoter comprising sequence elements from two or more different promoters.

In some embodiments, the promoter is a constitutively active promoter. Constitutive promoters include any constitutive promoter described herein or known to one of ordinary skill in the art. Non-limiting examples of constitutive promoters include the immediate early cytomegalovirus (CMV) promoter, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as mammalian gene promoters such as, but not limited to, the elongation Factor-la (EF-la) the actin promoter, the myosin promoter, the hemoglobin promoter, and the creatine kinase promoter.

Inducible promoters are also contemplated herein. An “inducible promoter” refers to a promoter that is characterized by regulating (e.g., initiating or activating) transcriptional activity when in the presence of, influenced by or contacted by an inducer signal.

In some embodiments, the promoter is a tissue-specific promoter. A “tissue-specific promoter”, as used herein, refers to promoters that preferentially or selectively function in a specific type of tissue. In some embodiments, a tissue-specific promoter is not able to drive the expression of the genes in other types of tissues. In some embodiments, a promoter that may be used in accordance with the present disclosure is a skeletal muscle-specific promoter. In some embodiments, the skeletal muscle-specific promoter is the CK6 promoter, CK8 promoter, or skeletal α-actin promoter. In some embodiments, the skeletal muscle-specific promoter comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a promoter that may be used in accordance with the present disclosure is a cardiac-specific promoter. In some embodiments, the cardiac-specific promoter is the cardiac troponin C promoter, the cardiac troponin I promoter, or the cardiac troponin T (cTnT) promoter. In some embodiments, the cardiac-specific promoter is the cTnT promoter.

In some embodiments, the engineered nucleic acids of the present disclosure further comprise one or more enhancer elements. In some embodiments, an enhancer element is a skeletal muscle alpha-actin enhancer. In some embodiments, the promoter is a skeletal muscle-specific promoter with skeletal muscle alpha-actin enhancer elements.

In some embodiments, the recombinant nucleic acids of the present disclosure further comprise additional regulatory sequences, including, without limitation, a 3′ untranslated region (3′UTR), and/or a poly-adenylation (polyA) signal sequence.

In some embodiments, a nucleotide sequence encoding a micro-dystrophin protein optimized for cardiac muscle is operably linked to a cardiac-specific promoter. In some embodiments, a nucleotide sequence encoding a micro-dystrophin protein optimized for skeletal muscle is operably linked to a skeletal muscle-specific promoter. In some embodiments, a nucleotide sequence encoding a micro-dystrophin protein optimized for cardiac muscle is operably linked to a weaker promoter. In some embodiments, the expression of a micro-dystrophin protein optimized for cardiac muscle is 5%, 10%, 155, 20%, 25%, 30%, 35%, 40%, 45%, or 50% lower than the expression of a micro-dystrophin protein optimized for skeletal muscle.

### Skeletal Muscle-Specific Promoter

In one aspect, the present disclosure provides minimal skeletal muscle-specific promoters based on the skeletal alpha-actin promoter. In some embodiments, a skeletal muscle-specific promoter of the disclosure does not comprise any element that mediates a response in cardiac muscle.

In some embodiments, a skeletal muscle-specific promoter of the disclosure is less than about 600 bp in length. In some embodiments, the skeletal muscle-specific promoter is less than about 550 bp in length. In some embodiments, the skeletal muscle-specific promoter is less than about 500 bp in length. In some embodiments, the skeletal muscle-specific promoter is less than about 400 bp in length.

In some embodiments, a skeletal muscle-specific promoter of the disclosure comprises one or more (e.g., 1, 2, 3, or more) copies of the skeletal muscle alpha-actin enhancer. In some embodiments, a skeletal muscle-specific promoter of the disclosure comprises one or more (e.g., 1, 2, 3, or more) copies of an enhancer that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to nucleotides 8-106 of SEQ ID NO: 76. In some embodiments, the skeletal alpha-actin enhancer comprises nucleotides 8-106 of SEQ ID NO: 76. In some embodiments, the skeletal alpha-actin enhancer consists essentially of nucleotides 8-106 of SEQ ID NO: 76. In some embodiments, the skeletal alpha-actin enhancer consists of nucleotides 8-106 of SEQ ID NO: 76. In some embodiments, a skeletal muscle-specific promoter of the disclosure comprises one or more (e.g., 1, 2, 3, or more) copies of an enhancer that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 76. In some embodiments, the skeletal alpha-actin enhancer comprises SEQ ID NO: 76. In some embodiments, the skeletal alpha-actin enhancer consists essentially of SEQ ID NO: 76. In some embodiments, the skeletal alpha-actin enhancer consists of SEQ ID NO: 76.

In some embodiments, a skeletal muscle-specific promoter of the disclosure comprises two copies of the skeletal muscle alpha-actin enhancer. In some embodiments, a skeletal muscle-specific promoter of the disclosure comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to nucleotides 8-205 of SEQ ID NO: 79. In some embodiments, the skeletal alpha-actin enhancer comprises nucleotides 8-205 of SEQ ID NO: 79. In some embodiments, the skeletal alpha-actin enhancer consists essentially of nucleotides 8-205 of SEQ ID NO: 79. In some embodiments, the skeletal alpha-actin enhancer consists of nucleotides 8-205 of SEQ ID NO: 79. In some embodiments, a skeletal muscle-specific promoter of the disclosure comprises an enhancer that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 79. In some embodiments, a skeletal muscle-specific promoter of the disclosure comprises an enhancer that consists essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 79. In some embodiments, a skeletal muscle-specific promoter of the disclosure comprises an enhancer that consists of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 79.

In some embodiments, a skeletal muscle-specific promoter of the disclosure comprises a core promoter sequence that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to nucleotides 1-274 of SEQ ID NO: 77. In some embodiments, the core promoter sequence comprises nucleotides 1-274 of SEQ ID NO: 77. In some embodiments, the core promoter sequence consists essentially of nucleotides 1-274 of SEQ ID NO: 77. In some embodiments, the core promoter sequence consists of nucleotides 1-274 of SEQ ID NO: 77. In some embodiments, a skeletal muscle-specific promoter of the disclosure comprises a core promoter sequence plus Kozak consensus sequence that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 77. In some embodiments, the core promoter sequence plus Kozak consensus sequence comprises SEQ ID NO: 77. In some embodiments, the core promoter sequence plus a Kozak consensus sequence consists essentially of SEQ ID NO: 77. In some embodiments, the core promoter sequence plus a Kozak consensus sequence consists of SEQ ID NO: 77.

In some embodiments, a skeletal muscle-specific promoter of the disclosure comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a skeletal muscle-specific promoter of the disclosure consists essentially of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a skeletal muscle-specific promoter of the disclosure consists of a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 78 or SEQ ID NO: 80. In some embodiments, a skeletal muscle-specific promoter of the disclosure comprises the nucleotide sequence of SEQ ID NO: 78 or SEQ ID NO: 80. In some embodiments, a skeletal muscle-specific promoter of the disclosure consists essentially of the nucleotide sequence of SEQ ID NO: 78 or SEQ ID NO: 80. In some embodiments, a skeletal muscle-specific promoter of the disclosure consists of the nucleotide sequence of SEQ ID NO: 78 or SEQ ID NO: 80.

A skeletal muscle-specific promoter of the disclosure may be used to express any gene of interest in skeletal muscle. Thus, a skeletal muscle-specific promoter of the disclosure may be operably linked to any gene of interest, to express the gene of interest in skeletal muscle. In some embodiments, the gene of interest is a micro-dystrophin gene of the disclosure.

A skeletal muscle-specific promoter of the disclosure may be used may be used for gene therapy for skeletal muscle disorders. In some embodiments, the skeletal muscle disorder is a muscular dystrophy. In some embodiments, the muscular dystrophy is DMD.

### Vectors

In some aspects, the present disclosure provides vectors comprising the recombinant nucleic acids described herein. A vector is any nucleic acid that may be used as a vehicle to deliver exogenous (foreign) genetic material to a cell. A vector, in some embodiments, is a DNA sequence that includes an insert (e.g., a nucleotide sequence encoding a micro-dystrophin protein and a larger sequence that serves as the backbone of the vector. Non-limiting examples of vectors include plasmids, viruses/viral vectors, phagemids, cosmids (comprising a plasmid and Lambda phage cos sequences), and artificial chromosomes, any of which may be used as provided herein. In some embodiments, the vector is a viral vector, such as a viral particle. In some embodiments, the viral vector is an adenovirus, adeno associated virus (AAV), γ-retrovirus, HSV, lentivirus, or Sendai virus vector. In some embodiments, the viral vector is an recombinant AAV (rAAV) vector.

In some embodiments, a nucleic acid of the disclosure is flanked by AAV ITRs for packaging into an rAAV vector.

The phrase rAAV vector can include a rAAV genome comprising the gene of interest flanked by AAV ITRs, and an rAAV particle comprising an rAAV genome encapsidated with rAAV capsid proteins.

The ITR sequences may be derived from any AAV serotype (e.g., 1, 2, 3, 4, 5, 6, 7, 8, 9, or 10) or may be derived from more than one serotype. In some embodiments, the ITR sequences are the same serotype as the capsid (e.g., AAV8 ITR sequences and AAV8 capsid, etc.). In some embodiments, the ITR sequences are of a different serotype from the capsid.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996 Nov. 26; 93 (24): 14082-7; and Curtis A. Machida. Methods in Molecular Medicine™. Viral Vectors for Gene Therapy Methods and Protocols. 10.1385/1-59259-304-6:201 @ Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

Further provided herein are rAAV viral particles or rAAV preparations containing such particles. The rAAV particles comprise a viral capsid and an rAAV genome comprising the gene of interest flanked by AAV ITRs, which is encapsidated by the viral capsid. Methods of producing rAAV particles are known in the art and are commercially available (see, e.g., Zolotukhin et al. Production and purification of serotype 1, 2, and 5 recombinant adeno-associated viral vectors. Methods 28 (2002) 158-167; and U.S. Patent Application Publication Numbers US 2007/0015238 and US 2012/0322861, which are incorporated herein by reference; and plasmids and kits available from ATCC and Cell Biolabs, Inc.). For example, a plasmid containing the rAAV genome comprising the gene of interest flanked by AAV ITRs may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

The rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV an rAAV particle refers to the serotype of the capsid proteins of the recombinant virus. Non-limiting examples of derivatives and pseudotypes include AAVrh.10, AAVrh.74, AAV2/1, AAV2/5, AAV2/6, AAV2/8, AAV2/9, AAV2-AAV3 hybrid, AAVhu.14, AAV3a/3b, AAVrh32.33. AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6 (Y445F/Y731F), AAV2.5T. AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20 (4): 699-708. doi: 10.1038/mt.2011.287. Epub 2012 Jan. 24. The AAV vector toolkit: poised at the clinical crossroads. Asokan A1, Schaffer D V, Samulski R J). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001). In some embodiments, the capsid of any of the herein disclosed rAAV particles is of the serotype AAV1, AAV2, AAV3, AAV6, AAV7, AAV8, AAVrh.74, AAVrh.10, AAV2/6 or AAV9.

In some embodiments, a nucleotide sequence encoding a micro-dystrophin protein optimized for cardiac muscle and a nucleotide sequence encoding a micro-dystrophin protein optimized for skeletal muscle are packaged in rAAV particles of the same serotype. In some embodiments, the micro-dystrophin protein comprises one or more utrophin protein domains.

In some embodiments, a nucleotide sequence encoding a micro-dystrophin protein optimized for cardiac muscle and a nucleotide sequence encoding a micro-dystrophin protein optimized for skeletal muscle are packaged in rAAV particles of different serotypes. In some embodiments, the micro-dystrophin protein comprises one or more utrophin protein domains.

In some embodiments, a nucleotide sequence encoding a micro-dystrophin protein optimized for cardiac muscle is packaged in an rAAV particle of the serotype AAV1, AAV8, AAV9, AAVrh.74, or AAVrh.10. In some embodiments, a nucleotide sequence encoding a micro-dystrophin protein optimized for cardiac muscle is packaged in an rAAV particle of the serotype AAV9, AAVrh.74, or AAVrh.10. In some embodiments, a nucleotide sequence encoding a micro-dystrophin protein optimized for skeletal muscle is packaged in an rAAV particle of the serotype AAV1, AAV6, AAV7, AAV8, or AAV9. In some embodiments, a nucleotide sequence encoding a micro-dystrophin protein optimized for skeletal muscle is packaged in an rAAV particle of the serotype AAV8. In some embodiments, the micro-dystrophin protein comprises one or more utrophin protein domains.

In some aspects, the methods described herein comprise expressing a micro-dystrophin protein in cardiac or skeletal muscle. The vectors provided herein may be used for gene therapy for treating skeletal muscle disorders in a subject in need thereof. In some embodiments, the vectors provided herein may be used for gene therapy for treating a muscular dystrophy (e.g., DMD) in a subject in need thereof.

### Methods of Use

In some aspects, the present disclosure provides methods of treating DMD.

In one aspect, the present disclosure provides a method of treating DMD in a subject in need thereof, comprising administering to the subject an effective amount of a micro-dystrophin protein of the disclosure. In some embodiments, the micro-dystrophin protein comprises one or more utrophin protein domains.

In another aspect, the present disclosure provides a gene therapy for treating DMD in a subject in need thereof. Accordingly, the present disclosure provides a method of treating DMD in a subject in need thereof, comprising administering to the subject an effective amount of a nucleic acid molecule encoding a micro-dystrophin protein. In some embodiments, the micro-dystrophin protein comprises one or more utrophin protein domains.

In some embodiments, the present disclosure provides a method of treating DMD in a subject in need thereof, comprising administering to the subject an effective amount of a nucleic acid molecule encoding a micro-dystrophin protein optimized for cardiac muscle and an effective amount of a nucleic acid molecule encoding a micro-dystrophin protein optimized for skeletal muscle. In some embodiments, the micro-dystrophin protein comprises one or more utrophin protein domains.

In some embodiments, the present disclosure provides a method of treating DMD in a subject in need thereof, comprising administering to the subject an effective amount of a nucleic acid molecule encoding a micro-dystrophin protein optimized for cardiac muscle to cardiac muscle and an effective amount of a nucleic acid molecule encoding a micro-dystrophin protein optimized for skeletal muscle to skeletal muscle. In some embodiments, the micro-dystrophin protein comprises one or more utrophin protein domains.

In some embodiments, the present disclosure provides a method of treating DMD in a subject in need thereof, the method comprising delivering to the subject a first gene therapy vector (e.g., a first rAAV particle) comprising a nucleotide sequence encoding a first micro-dystrophin protein to cardiac muscle and a second gene therapy vector (e.g., a second rAAV particle) comprising a nucleotide sequence encoding a second micro-dystrophin to skeletal muscle. In some embodiments, the first recombinant adeno-associated virus (rAAV) particle comprising a nucleotide sequence encoding a first micro-dystrophin protein to cardiac muscle also comprises a nucleotide sequence encoding a first utrophin protein region. In some embodiments, the second rAAV particle comprising a nucleotide sequence encoding a second micro-dystrophin to skeletal muscle also comprises a nucleotide sequence encoding a second utrophin protein region. In some embodiments, the first recombinant adeno-associated virus (rAAV) particle comprises a nucleotide sequence encoding a first chimeric protein comprising a first micro-dystrophin protein and a first utrophin protein region to cardiac muscle. In some embodiments, the second recombinant adeno-associated virus (rAAV) particle comprises a nucleotide sequence encoding a second chimeric protein comprising a second micro-dystrophin protein and a second utrophin protein region to skeletal muscle. In some embodiments, the first or second micro-dystrophin protein comprises one or more utrophin protein domains.

In some embodiments, methods are provided for transducing a cardiac muscle cell with the first rAAV particle of the disclosure. In some embodiments, methods are provided for transducing a skeletal muscle cell with the second rAAV particle of the disclosure. In some embodiments, methods are provided for transducing a cardiac muscle cell with any rAAV particle of the disclosure. In some embodiments, methods are provided for transducing a skeletal muscle cell with any rAAV particle of the disclosure.

The present disclosure thus contemplates methods of expressing one or more micro-dystrophin proteins in a subject for treating DMD, the method comprising administering to a subject in need thereof an effective amount of one or more nucleic acids of the disclosure.

In some embodiments, additional gene therapy approaches are combined with micro-dystrophin.

The terms “subject.” and “patient,” are used interchangeably herein. In some embodiments, a subject is a mammal, such as a human, a nonhuman primate, a dog, a cat, a horse, a sheep, a poultry, a cow, a pig, a mouse, a rat, a rodent, or a goat. In some embodiments, the subject and mammal is a human.

An “effective amount” of the compositions of the disclosure generally refers to an amount sufficient to elicit the desired biological response, e.g., express the micro-dystrophin protein in a target cell, treat DMD, etc. As will be appreciated by those of ordinary skill in this art, the effective amount of an agent described herein may vary depending on such factors as the condition being treated, the mode of administration, and the age, body composition, and health of the subject. Suitable dosage ranges are readily determinable by one skilled in the art.

The terms “treat”, “treating”, “treatment”, and “therapy” encompass an action that occurs while a subject is suffering from a condition which reduces the severity of the condition (or a symptom associated with the condition) or retards or slows the progression of the condition (or a symptom associated with the condition).

### Compositions

In some aspects, the present disclosure provides compositions comprising the polypeptides, nucleic acids, or vectors disclosed herein. For administration to a subject, the polypeptides, engineered nucleic acids, or vectors disclosed herein may be formulated in a composition. In some embodiments, the composition further comprises additional agents (e.g., for specific delivery, increasing half-life, or other therapeutic agents).

In some embodiments, the composition is a pharmaceutical composition. In some embodiments, the composition further comprises a pharmaceutically acceptable carrier. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” is a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Nucleic acids, in some embodiments, may be formulated in a non-viral delivery vehicle. Non-limiting examples of non-viral delivery vehicles include nanoparticles, such as nanocapsules and nanospheres. Sec, e.g., Sing, R et al. Exp Mol Pathol. 2009; 86 (3): 215-223. A nanocapsule is often comprised of a polymeric shell encapsulating an agent. Nanospheres are often comprised of a solid polymeric matrix throughout which the agent is dispersed. In some embodiments, the nanoparticle is a lipid particle, such as a liposome. See, e.g., Puri, A et al. Crit Rev Ther Drug Carrier Syst. 2009; 26 (6): 523-80. The term ‘nanoparticle’ also encompasses microparticles, such as microcapsules and microspheres.

Methods developed for making particles for delivery of encapsulated agents are described in the literature (for example, please see Doubrow, M., Ed., “Microcapsules and Nanoparticles in Medicine and Pharmacy.” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the engineered nucleic acids disclosed herein may be found, for example, in Remington's Pharmaceutical Sciences, 18th edition, Mack Publishing Co., Easton, Pa (1990) (incorporated herein by reference in its entirety).

### Methods of Administration

Any of the polypeptides, nucleic acids, vectors, or compositions disclosed herein may be administered to a subject.

Suitable routes of administration include, without limitation, intravenous, intranasal, intramuscular, intrathecal, or subcutaneous. In some embodiments, a polypeptide, engineered nucleic acid, vector, or composition of the disclosure is administered intravenously, subcutaneously, intramuscularly intrathecally or intranasally. In some embodiments, a polypeptide, nucleic acid, vector, or composition of the disclosure is administered directly (e.g., by direct injection) to one or more cells (e.g., cardiac or skeletal muscle cells), tissues (e.g., cardiac or skeletal muscle), or organs (e.g., heart). Other routes of administration are contemplated herein. The administration route can be changed depending on a number of factors, including the desired cell, tissue, or organ.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., oral, parenteral, intravenous, intranasal, intraarticular, and intramuscular administration and formulation.

In one aspect, the present disclosure relates to methods of achieving localized delivery of a gene therapy vector (e.g., an rAAV vector) to a muscle of a subject. Localized delivery to a muscle can be achieved through different methods including, but not limited to, catheter-based methods, local injections, local injection devices, microneedles or drug-eluting stents or drug-eluting implants. In one aspect, the present disclosure relates to catheter-based methods of delivering gene therapy vectors to a muscle. Examples of catheters include, but are not limited to, guiding catheters, microporous infusion catheters, balloon catheters, porous balloon catheters, microporous balloon catheters, retractable-needle catheters, over-the-needle (OTN) catheters, iontophoretic catheters or butterfly catheters. In some embodiments, the muscle is cardiac muscle. In some embodiments, the muscle is skeletal muscle. Non-limiting examples of skeletal muscles include arm muscles, hand muscles, shoulder muscles, chest muscles, neck muscles, muscles of the larynx, scalp muscles, eye muscles, hip muscles, leg muscles and thigh muscles.

In some embodiments, catheter delivery allows the combined delivery of one or more gene therapy vectors to the cardiac muscle and to one or more skeletal muscle(s). In some embodiments, the gene therapy vector delivered to the cardiac muscle is the same as the gene therapy vector delivered to the skeletal muscle(s). In some embodiments, the gene therapy vector delivered to the cardiac muscle is different from the gene therapy vector delivered to the skeletal muscle(s). In some embodiments, the catheter has multiple lumens for the delivery of the different gene therapy vectors. In some embodiments, the catheter has a single lumen. In some embodiments, the different gene therapy vectors are sequentially introduced as the catheter is moved to different arteries. In some embodiments, the combined delivery to cardiac and skeletal muscles comprises advancing a catheter to heart, delivering a gene therapy vector into the left and right coronary arteries, retracting the catheter to the aortic arch, and delivering a gene therapy vector to arteries that irrigate the skeletal muscles (e.g., the subclavian and/or carotid arteries). In some embodiments, the method further comprises retracting the catheter into the descending aorta and delivering the gene therapy to skeletal muscle via descending aortic branches.

In some embodiments, the present disclosure relates to the delivery of a gene therapy vector to an artery via a catheter. Non-limiting examples of arteries include a femoral artery, a subclavian artery, a carotid artery, an axillary artery, a brachial artery, a radial artery, an ulnar artery, an iliac artery, a popliteal artery, a tibial artery, a dorsalis pedis artery and an aorta. In some embodiments, the gene therapy vector is delivered to a subclavian and/or a carotid artery. In some embodiments, the catheter is first introduced into the femoral artery and advanced to the subclavian and/or carotid arteries. In some embodiments, the catheter is then retracted into the descending aorta to deliver the gene therapy vector to skeletal muscle via descending aortic branches.

In some embodiments, the methods described herein relate to the delivery of a first rAAV to cardiac muscle and a second rAAV to a skeletal muscle, comprising: (i) introducing a catheter into the femoral artery and advancing to the heart; (ii) delivering the first gene therapy vector into the left and right coronary arteries; (iii) retracting the catheter to the aortic arch; and (iv) delivering the second gene therapy vector to the subclavian and/or carotid arteries. In some embodiments, the first and second rAAVs have different capsid serotypes. In some embodiments, the first and second rAA Vs have the same capsid serotype. In some embodiments, the first and second rAA Vs carry the same therapeutic gene. In some embodiments, the first and second rAA Vs carry different therapeutic genes. In some embodiments, a first rAAV carries a micro-dystrophin gene optimized for cardiac muscle and the second rAAV carries a micro-dystrophin gene optimized for skeletal muscle.

In some embodiments, a vasodilator is administered to the subject prior to, or simultaneously with, the introduction of the catheter into an artery for gene therapy vector delivery. Vasodilators are medications that open (dilate) blood vessels. Non-limiting examples of vasodilators are arterial dilators, venous dilators, mixed dilators, nitroprusside, nitroglycerin, nitric oxide, hydralazine, allicin, nitrates, isosorbide dinitrate, isosorbide mononitrate, erythrityl tetranitrate, pentaerythritol tetranitrate, sodium nitroprusside, alpha-adrenoceptor antagonists (alpha blockers), al-adrenoceptor antagonists (e.g., prazosin, terazosin, doxazosin, trimazosin), phentolamine, phenoxybenzamine, sympatholytics, angiotensin converting enzyme (ACE) inhibitors (e.g., benazepril, captopril, enalapril, fosinopril, lisinopril, moexipril, quinapril, ramipril), angiotensin receptor blockers (ARBs) (e.g., candesartan, eprosartan, irbesartan, losartan, olmesartan, telmisartan, azilsartan, valsartan), alpha-2-agonists (e.g., α-methyldopa, clonidine), alpha agonist of the alpha-2 adrenergic receptor (e.g., guanabenz, guanfacine) beta2-adrenoceptor agonists (B2-agonists), beta-adrenoceptor agonist (isoprenaline), beta-1-adrenergic agonist (e.g., dobutamine), calcium-channel blockers (CCBs) (e.g., amlodipine, felodipine, isradipine, nicardipine, nifedipine, nimodipine, nitrendipine, verapamil, diltiazem), centrally acting sympatholytics, direct acting vasodilators, endothelin receptor antagonists (e.g., bosentan, ambrisentan), ganglionic blockers (e.g. trimethaphan camsylate), nitrodilators, phosphodiesterase inhibitors, potassium-channel openers (e.g., minoxidil), renin inhibitors (e.g., aliskiren), PDE3 inhibitors (e.g., milrinone, inamrinone, amrinone, cilostazol), PDE5 inhibitors (e.g., sildenafil, tadalafil), epinephrine, norepinephrine, or dopamine. In some embodiments, the vasodilator is adenosine. In some embodiments, the vasodilator is a PDE5 inhibitor. In some embodiments, the PDE5 inhibitor is sildenafil or tadalafil. In some embodiments, the PDE5 inhibitor is sildenafil. In some embodiments, the vasodilator is administered 0, 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150 or 180 minutes prior to the delivery of the gene therapy vector.

In some embodiments, administration of the vasodilator is through intravascular injection. In some embodiments, administration of the vasodilator is through intramuscular injection. In some embodiments, administration of the vasodilator is through ingestion. In some embodiments, administration of the vasodilator is through topical application. s

In some embodiments, the methods of the present disclosure further comprise inflating a blood pressure cuff on each limb of the subject at the time of delivery of the gene therapy vector.

In some embodiments, the nucleic acids of the disclosure are delivered via an AAV vector. In some embodiments, the number of AAV particles administered to a subject may be on the order ranging from about 10⁹ to about 10¹⁶ particles, or any values in between, such as for example, about 10⁹, 10¹⁰, 10¹¹10¹², 10¹³, 10¹⁴, 10¹⁵, or 10¹⁶ particles. In some embodiments, the number of AAV particles administered to a subject may be on the order ranging from about 10⁹ to about 10¹⁶ vector genomes (vgs), or any values in between, such as for example, about 10⁹, 10¹⁰, 10¹¹, 10¹², 10¹³, 10¹⁴, 10¹⁵, or 10¹⁶ vgs. The AAV particles can be administered as a single dose, or divided into two or more administrations as may be required to achieve therapy. In some embodiments, the two or more administrations are within 24 hours of each other.

### EXAMPLES

### Example 1. Micro-Dystrophin Constructs that are Optimized for Either Skeletal Muscle or Cardiac Muscle

Based on studies in mice with micro-dystrophin constructs that are currently in clinical trials, two discoveries were made. First, it was found that the affinity of the micro-dystrophin for its membrane complex is modulated by the inclusion of the first three spectrin-like repeats (FIG. 1). The inclusion of only R1 leads to relatively weak binding, while the presence of R1+R2 or R1+R2+R3 leads to a much higher apparent affinity for the complex.

Secondly, it was discovered that overexpression of a micro-dystrophin containing either R1+R2 or R1+R2+R3 leads to outcompeting utrophin at the cardiac membrane, while not affecting utrophin in skeletal muscle. This displacement of utrophin in the heart leads to a much more rapid onset of cardiomyopathy and heart failure than if no dystrophin is present. On the other hand, a R1 only containing micro-dystrophin does not outcompete utrophin for its membrane binding in the heart to the same extent. Accordingly, a micro-dystrophin that will be beneficial to the heart, can be designed based on including only R1 of the first three repeats.

In the case of skeletal muscle, it was found that the greater the affinity for the complex, the better, since utrophin localization is unaffected. Thus, for skeletal muscle, the ideal constructs contain either R1+R2 or R1+R2+R3. However, in some embodiments, in order to accommodate the full C-terminus, it may be necessary to exclude R2 and only include R1.

Lastly, the loss of regulation of ion channels, including the TRPC1, 3, 6 channels, leads to residual pathology in skeletal and cardiac muscles that the current micro-dystrophins do not address. Accordingly, in some embodiments, regions in the C-terminus of dystrophin, including the syntrophin binding sites and/or the coiled coil regions that interact with dystrobrevin (FIG. 1), are included in micro-dystrophin.

The following exemplary micro-dystrophin constructs were designed for AAV delivery in view of the above findings. In some embodiments, the micro-dystrophin constructs designed for skeletal muscle are used with a promoter that expresses only in skeletal muscle and not in the heart. In some embodiments, cardiac-specific expression is achieved with the truncated cTnT promoter. In some embodiments, a smaller cTnT promoter is generated to allow use of the largest cardiac micro-dystrophins described below. FIG. 2 shows the order of Repeats (R) and hinges (H), as well as the cysteine-rich portion of the C-terminus (CR), which is commonly included in micro-dystrophins and referred to as the truncated C-terminus, which is then followed by the rest of the C-terminus (CT).

TABLE 1
Cardiac micro-dystrophins

| | Size (amino |
|---|---|
| Elements included in construct | acid number) |
| Nterm-R1_R17-R19_R24 + H4 + trCterm | 1263 |
| Nterm-R1_R17-R19_H4 to 1^{st} coiled coil and proline | 1286 |
| rich region that follows | |
| Nterm-R1_R17-R19_H4 to end of syntrophin region | 1223 |
| Nterm-R1_R17-R19_R24 to 1^{st} coiled coil and proline | 1407 |
| rich region that follows | |
| Nterm-R1_R17-R19_H4 to end of dystrophin | 1418 |
| Nterm-R1_R17-R19_R24 to end of dystrophin | 1539 |

TABLE 2
Skeletal Muscle micro-dystrophins

| | Size (amino |
|---|---|
| Elements included in construct | acid number) |
| Nterm-R2_R16-R17_R24 + H4 + trCterm | 1277 |
| Nterm-R2_R16-R17_H4 to end of syntrophin region | 1237 |
| Nterm-R2_R16-R17_H4 to 1^{st} coiled coil and proline | 1300 |
| rich region that follows | |
| Nterm-R2_R16-R17_R24 to 1^{st} coiled coil and proline | 1421 |
| rich region that follows | |
| Nterm-R2_R23, R24 to end of dystrophin | 1431 |
| Nterm-R2_R16-R17_H4 to end | 1432 |
| Nterm-R1_R16-R17_R24 to end | 1448 |
| Nterm-R2_R16-R17_R24 to end | 1553 |
| Nterm-R2_R16-R17_R24 to end of Syntrophin region | 1358 |

In the tables above, “-” is used to show the end points of domains that are continuously linked while “_” is used to indicate a deletion between the domains. For example, Nterm-R1_R17-R19_R24 to trCterm indicates that the micro-dystrophin comprises the N-terminus region up to and including R1 linked to R17 up to and including R19 linked to R24 up to and including the truncated C-terminus of dystrophin (i.e., the region between R1 and R17, the region between R19 and R24, and the region after the truncated C-terminus are deleted).

The amino acid sequences of modules to build the micro-dystrophin proteins described in this example are provided as SEQ ID NOs: 40-52, and 186-187 below. The amino acids of the micro-dystrophin constructs in Tables 1 and 2 are provided as SEQ ID Nos. 1-6 and 13-21 elsewhere in the description. In some embodiments, the present disclosure provides micro-dystrophin proteins comprising one or more amino acid sequences selected from the group consisting of SEQ ID NOs: 40-52, and 186-187.

N-terminus through Hinge 1 (325 amino acids)
(SEQ ID NO: 40)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAP
Repeat 1 (126 amino acids)
(SEQ ID NO: 41)
EDKSFGSSLMESEVNLDRYQTALEEVLSWLLSAEDTLQAQGEISNDVEVVKDQFHTHEGYMMDLTAHQGRVGNIL
QLGSKLIGTGKLSEDEETEVQEQMNLLNSRWECLRVASMEKQSNLHRVLMD
Repeat 2 (105 amino acids)
(SEQ ID NO: 42)
LQNQKLKELNDWLTKTEERTRKMEEEPLGPDLEDLKRQVQQHKVLQEDLEQEQVRVNSLTHMVVVVDESSGDHAT
AALEEQLKVLGDRWANICRWTEDRWVLLQD
Repeat 3 + 4 residues of H2 (118 amino acids)
(SEQ ID NO: 43)
ILLKWQRLTEEQCLFSAWLSEKEDAVNKIHTTGFKDQNEMLSSLQKLAVLKADLEKKKQSMGKLYSLKQDLLSTL
KNKSVTQKTEAWLDNFARCWDNLVQKLEKSTAQISQAVTTTQP
Repeat 16 + linker (114 + 9 amino acids = 123 amino acids)
(SEQ ID NO: 44)
HTVREETMMVMTEDMPLEISYVPSTYLTEITHVSQALLEVEQLLNAPDLCAKDFEDLFKQEESLKNIKDSLQQSS
GRIDIIHSKKTAALQSATPVERVKLQEALSQLDFQWEKVNKMYKDRQG
Repeat 17 (113amino acids)
(SEQ ID NO: 45)
RFDRSVEKWRRFHYDIKIFNQWLTEAEQFLRKTQIPENWEHAKYKWYLKELQDGIGQRQTVVRTLNATGEEIIQQ
SSKTDASILQEKLGSLNLRWQEVCKQLSDRKKRLEEQK
Repeats 17-19 (327 aa)
(SEQ ID NO: 46)
RFDRSVEKWRRFHYDIKIFNQWLTEAEQFLRKTQIPENWEHAKYKWYLKELQDGIGQRQTVVRTLNATGEEIIQQ
SSKTDASILQEKLGSLNLRWQEVCKQLSDRKKRLEEQKNILSEFQRDLNEFVLWLEEADNIASIPLEPGKEQQLK
EKLEQVKLLVEELPLRQGILKQLNETGGPVLVSAPISPEEQDKLENKLKQTNLQWIKVSRALPEKQGEIEAQIKD
LGQLEKKLEDLEEQLNHLLLWLSPIRNQLEIYNQPNQEGPFDVKETEIAVQAKQPDVEEILSKGQHLYKEKPATQ
PVKRKLEDLSSEWKAVNRLLQELRAKQ
Repeats 17-19 (324 aa)
(SEQ ID NO: 186)
RSVEKWRRFHYDIKIFNQWLTEAEQFLRKTQIPENWEHAKYKWYLKELQDGIGQRQTVVRTLNATGEEII
QQSSKTDASILQEKLGSLNLRWQEVCKQLSDRKKRLEEQKNILSEFQRDLNEFVLWLEEADNIASIPLEP
GKEQQLKEKLEQVKLLVEELPLRQGILKQLNETGGPVLVSAPISPEEQDKLENKLKQTNLQWIKVSRALP
EKQGEIEAQIKDLGQLEKKLEDLEEQLNHLLLWLSPIRNQLEIYNQPNQEGPFDVKETEIAVQAKQPDVE
EILSKGQHLYKEKPATQPVKRKLEDLSSEWKAVNRLLQELRAKQ
Repeat 23 (114 aa)
(SEQ ID NO: 47)
HLSLQELLVWLQLKDDELSRQAPIGGDFPAVQKQNDVHRAFKRELKTKEPVIMSTLETVRIFLTEQPLEGLEKLY
QEPRELPPEERAQNVTRLLRKQAEEVNTEWEKLNLHSAD
Repeat 23 (123 amino acids)
(SEQ ID NO: 187)
ASSDQWKRLHLSLQELLVWLQLKDDELSRQAPIGGDFPAVQKQNDVHRAFKRELKTKEPVIMSTLETVRIFLTEQ
PLEGLEKLYQEPRELPPEERAQNVTRLLRKQAEEVNTEWEKLNLHSAD
Repeat 24 (121 amino acids)
(SEQ ID NO: 48)
WQRKIDETLERLQELQEATDELDLKLRQAEVIKGSWQPVGDLLIDSLQDHLEKVKALRGEIAPLKENVSHVNDLA
RQLTTLGIQLSPYNLSTLEDLNTRWKLLQVAVEDRVRQLHEAHRDF
Hinge 4 + truncated C terminus (364 amino acids)
(SEQ ID NO: 49)
GPASQHELSTSVQGPWERAISPNKVPYYINHETQTTCWDHPKMTELYQSLADLNNVRESAYRTAMKLRRLQKALC
LDLLSLSAACDALDQHNLKQNDQPMDILQIINCLTTIYDRLEQEHNNLVNVPLCVDMCLNWLLNVYDTGRTGRIR
VLSFKTGIISLCKAHLEDKYRYLFKQVASSTGFCDQRRLGLLLHDSIQIPRQLGEVASFGGSNIEPSVRSCFQFA
NNKPEIEAALFLDWMRLEPQSMVWLPVLHRVAAAETAKHQAKCNICKECPIIGFRYRSLKHFNYDICQSCFFSGR
VAKGHKMHYPMVEYCTPTTSGEDVRDFAKVLKNKFRTKRYFAKHPRMGYLPVQTVLEGDNMETP
Syntrophin binding sites (81 amino acids)
(SEQ ID NO: 50)
VTLINFWPVDSAPASSPQLSHDDTHSRIEHYASRLAEMENSNGSYLNDSISPNESIDDEHLLIQHYCQSLNQDSP
LSQPRS
Coiled coil region (195 amino acids)
1st coiled coil adds 47aa; including proline rich region adds 16 more aa
(total of 63aa)
(SEQ ID NO: 51)
PAQILISLESEERGELERILADLEEENRNLQAEYDRLKQQHEHKGLSPLPSPPEMMPTSPQSP
2nd coiled coil region to end of dystrophin; including 2nd coiled coil
adds another 61aa and region after adds 71 more aa (total of 132aa)
(SEQ ID NO: 52)
RDAELIAEAKLLRQHKGRLEARMQILEDHNKQLESQLHRLRQLLEQPQAEAKVNGTTVSSPSTSLQRSDSSQPML
LRVVGSQTSDSMGEEDLLSPPQDTSTGLEEVMEQLNNSFPSSRGRNTPGKPMREDTM

The nucleotide sequences of modules to build the micro-dystrophin genes described in this example are provided below as SEQ ID NOs: 53-75, and 188-190. The nucleotide sequences of the micro-dystrophin constructs in Tables 1 and 2 are provided as SEQ ID Nos. 7-12 and 22-39 elsewhere in the description. In some embodiments, the present disclosure provides nucleic acid constructs comprising one or more of SEQ ID NOs: 53-75, and 188-190.

N-terminus through Hinge 1
Human sequence without codon alterations:
(SEQ ID NO: 53)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
Human sequence with codon optimization:
(SEQ ID NO: 54)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
Repeat 1
Human sequence without codon alterations:
(SEQ ID NO: 55)
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GAT
Human sequence with codon optimization:
(SEQ ID NO: 56)
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GAC
Repeat 2
Human sequence without codon alterations:
(SEQ ID NO: 57)
CTCCAGAATCAGAAACTGAAAGAGTTGAATGACTGGCTAACAAAAACAGAAGAAAGAACAAGGAAAATGGAGGAA
GAGCCTCTTGGACCTGATCTTGAAGACCTAAAACGCCAAGTACAACAACATAAGGTGCTTCAAGAAGATCTAGAA
CAAGAACAAGTCAGGGTCAATTCTCTCACTCACATGGTGGTGGTAGTTGATGAATCTAGTGGAGATCACGCAACT
GCTGCTTTGGAAGAACAACTTAAGGTATTGGGAGATCGATGGGCAAACATCTGTAGATGGACAGAAGACCGCTGG
GTTCTTTTACAAGAC
Human sequence with codon optimization:
(SEQ ID NO: 58)
CTGCAGAACCAGAAGCTGAAGGAGCTGAACGACTGGCTGACCAAGACCGAGGAGCGGACCCGGAAGATGGAGGAG
GAGCCCCTGGGCCCCGACCTGGAGGACCTGAAGCGGCAGGTGCAGCAGCACAAGGTGCTGCAGGAGGACCTGGAG
CAGGAGCAGGTGCGGGTGAACAGCCTGACCCACATGGTGGTGGTGGTGGACGAGAGCAGCGGCGACCACGCCACC
GCCGCCCTGGAGGAGCAGCTGAAGGTGCTGGGCGACCGGTGGGCCAACATCTGCCGGTGGACCGAGGACCGGTGG
GTGCTGCTGCAGGAC
Repeat 16 + linker
Human sequence without codon alterations:
(SEQ ID NO: 59)
CACACTGTCCGTGAAGAAACGATGATGGTGATGACTGAAGACATGCCTTTGGAAATTTCTTATGTGCCTTCTACT
TATTTGACTGAAATCACTCATGTCTCACAAGCCCTATTAGAAGTGGAACAACTTCTCAATGCTCCTGACCTCTGT
GCTAAGGACTTTGAAGATCTCTTTAAGCAAGAGGAGTCTCTGAAGAATATAAAAGATAGTCTACAACAAAGCTCA
GGTCGGATTGACATTATTCATAGCAAGAAGACAGCAGCATTGCAAAGTGCAACGCCTGTGGAAAGGGTGAAGCTA
CAGGAAGCTCTCTCCCAGCTTGATTTCCAATGGGAAAAAGTTAACAAAATGTACAAGGACCGACAAGGG
Human sequence with codon optimization:
(SEQ ID NO: 60)
CACACCGTGCGGGAGGAGACCATGATGGTGATGACCGAGGACATGCCCCTGGAGATCAGCTACGTGCCCAGCACC
TACCTGACCGAGATCACCCACGTGAGCCAGGCCCTGCTGGAGGTGGAGCAGCTGCTGAACGCCCCCGACCTGTGC
GCCAAGGACTTCGAGGACCTGTTCAAGCAGGAGGAGAGCCTGAAGAACATCAAGGACAGCCTGCAGCAGAGCAGC
GGCCGGATCGACATCATCCACAGCAAGAAGACCGCCGCCCTGCAGAGCGCCACCCCCGTGGAGCGGGTGAAGCTG
CAGGAGGCCCTGAGCCAGCTGGACTTCCAGTGGGAGAAGGTGAACAAGATGTACAAGGACCGGCAGGGC
Repeat 17
Human sequence without codon alterations:
(SEQ ID NO: 61)
CGATTTGACAGATCTGTTGAGAAATGGCGGCGTTTTCATTATGATATAAAGATATTTAATCAGTGGCTAACAGAA
GCTGAACAGTTTCTCAGAAAGACACAAATTCCTGAGAATTGGGAACATGCTAAATACAAATGGTATCTTAAGGAA
CTCCAGGATGGCATTGGGCAGCGGCAAACTGTTGTCAGAACATTGAATGCAACTGGGGAAGAAATAATTCAGCAA
TCCTCAAAAACAGATGCCAGTATTCTACAGGAAAAATTGGGAAGCCTGAATCTGCGGTGGCAGGAGGTCTGCAAA
CAGCTGTCAGACAGAAAAAAGAGGCTAGAAGAACAAAAG
Human sequence with codon optimization:
(SEQ ID NO: 62)
CGGTTCGACCGGAGCGTGGAGAAGTGGCGGCGGTTCCACTACGACATCAAGATCTTCAACCAGTGGCTGACCGAG
GCCGAGCAGTTCCTGCGGAAGACCCAGATCCCCGAGAACTGGGAGCACGCCAAGTACAAGTGGTACCTGAAGGAG
CTGCAGGACGGCATCGGCCAGCGGCAGACCGTGGTGCGGACCCTGAACGCCACCGGCGAGGAGATCATCCAGCAG
AGCAGCAAGACCGACGCCAGCATCCTGCAGGAGAAGCTGGGCAGCCTGAACCTGCGGTGGCAGGAGGTGTGCAAG
CAGCTGAGCGACCGGAAGAAGCGGCTGGAGGAGCAGAAG
Repeats 17-19 (encoding 327 aa)
Human sequence without codon alterations:
(SEQ ID NO: 63)
CGATTTGACAGATCTGTTGAGAAATGGCGGCGTTTTCATTATGATATAAAGATATTTAATCAGTGGCTAACAGAA
GCTGAACAGTTTCTCAGAAAGACACAAATTCCTGAGAATTGGGAACATGCTAAATACAAATGGTATCTTAAGGAA
CTCCAGGATGGCATTGGGCAGCGGCAAACTGTTGTCAGAACATTGAATGCAACTGGGGAAGAAATAATTCAGCAA
TCCTCAAAAACAGATGCCAGTATTCTACAGGAAAAATTGGGAAGCCTGAATCTGCGGTGGCAGGAGGTCTGCAAA
CAGCTGTCAGACAGAAAAAAGAGGCTAGAAGAACAAAAGAATATCTTGTCAGAATTTCAAAGAGATTTAAATGAA
TTTGTTTTATGGTTGGAGGAAGCAGATAACATTGCTAGTATCCCACTTGAACCTGGAAAAGAGCAGCAACTAAAA
GAAAAGCTTGAGCAAGTCAAGTTACTGGTGGAAGAGTTGCCCCTGCGCCAGGGAATTCTCAAACAATTAAATGAA
ACTGGAGGACCCGTGCTTGTAAGTGCTCCCATAAGCCCAGAAGAGCAAGATAAACTTGAAAATAAGCTCAAGCAG
ACAAATCTCCAGTGGATAAAGGTTTCCAGAGCTTTACCTGAGAAACAAGGAGAAATTGAAGCTCAAATAAAAGAC
CTTGGGCAGCTTGAAAAAAAGCTTGAAGACCTTGAAGAGCAGTTAAATCATCTGCTGCTGTGGTTATCTCCTATT
AGGAATCAGTTGGAAATTTATAACCAACCAAACCAAGAAGGACCATTTGACGTTAAGGAAACTGAAATAGCAGTT
CAAGCTAAACAACCGGATGTGGAAGAGATTTTGTCTAAAGGGCAGCATTTGTACAAGGAAAAACCAGCCACTCAG
CCAGTGAAGAGGAAGTTAGAAGATCTGAGCTCTGAGTGGAAGGCGGTAAACCGTTTACTTCAAGAGCTGAGGGCA
AAGCAG
Repeats 17-19 (encoding 324 aa)
Human sequence without codon alterations:
(SEQ ID NO: 188)
AGATCTGTTGAGAAATGGCGGCGTTTTCATTATGATATAAAGATATTTAATCAGTGGCTAACAGAAGCTGAACAG
TTTCTCAGAAAGACACAAATTCCTGAGAATTGGGAACATGCTAAATACAAATGGTATCTTAAGGAACTCCAGGAT
GGCATTGGGCAGCGGCAAACTGTTGTCAGAACATTGAATGCAACTGGGGAAGAAATAATTCAGCAATCCTCAAAA
ACAGATGCCAGTATTCTACAGGAAAAATTGGGAAGCCTGAATCTGCGGTGGCAGGAGGTCTGCAAACAGCTGTCA
GACAGAAAAAAGAGGCTAGAAGAACAAAAGAATATCTTGTCAGAATTTCAAAGAGATTTAAATGAATTTGTTTTA
TGGTTGGAGGAAGCAGATAACATTGCTAGTATCCCACTTGAACCTGGAAAAGAGCAGCAACTAAAAGAAAAGCTT
GAGCAAGTCAAGTTACTGGTGGAAGAGTTGCCCCTGCGCCAGGGAATTCTCAAACAATTAAATGAAACTGGAGGA
CCCGTGCTTGTAAGTGCTCCCATAAGCCCAGAAGAGCAAGATAAACTTGAAAATAAGCTCAAGCAGACAAATCTC
CAGTGGATAAAGGTTTCCAGAGCTTTACCTGAGAAACAAGGAGAAATTGAAGCTCAAATAAAAGACCTTGGGCAG
CTTGAAAAAAAGCTTGAAGACCTTGAAGAGCAGTTAAATCATCTGCTGCTGTGGTTATCTCCTATTAGGAATCAG
TTGGAAATTTATAACCAACCAAACCAAGAAGGACCATTTGACGTTAAGGAAACTGAAATAGCAGTTCAAGCTAAA
CAACCGGATGTGGAAGAGATTTTGTCTAAAGGGCAGCATTTGTACAAGGAAAAACCAGCCACTCAGCCAGTGAAG
AGGAAGTTAGAAGATCTGAGCTCTGAGTGGAAGGCGGTAAACCGTTTACTTCAAGAGCTGAGGGCAAAGCAG
Repeat 23 (encoding 114 aa)
Human sequence without codon alterations:
(SEQ ID NO: 64)
CACCTTTCTCTGCAGGAACTTCTGGTGTGGCTACAGCTGAAAGATGATGAATTAAGCCGGCAGGCACCTATTGGA
GGCGACTTTCCAGCAGTTCAGAAGCAGAACGATGTACATAGGGCCTTCAAGAGGGAATTGAAAACTAAAGAACCT
GTAATCATGAGTACTCTTGAGACTGTACGAATATTTCTGACAGAGCAGCCTTTGGAAGGACTAGAGAAACTCTAC
CAGGAGCCCAGAGAGCTGCCTCCTGAGGAGAGAGCCCAGAATGTCACTCGGCTICTACGAAAGCAGGCTGAGGAG
GTCAATACTGAGTGGGAAAAATTGAACCTGCACTCCGCTGAC
Human Sequences with codon optimization:
(SEQ ID NO: 65)
CACCTGAGCCTGCAGGAGCTGCTGGTGTGGCTGCAGCTGAAGGACGACGAGCTGAGCCGGCAGGCCCCCATCGGC
GGCGACTTCCCCGCCGTGCAGAAGCAGAACGACGTGCACCGGGCCTTCAAGCGGGAGCTGAAGACCAAGGAGCCC
GTGATCATGAGCACCCTGGAGACCGTGCGGATCTTCCTGACCGAGCAGCCCCTGGAGGGCCTGGAGAAGCTGTAC
CAGGAGCCCCGGGAGCTGCCCCCCGAGGAGCGGGCCCAGAACGTGACCCGGCTGCTGCGGAAGCAGGCCGAGGAG
GTGAACACCGAGTGGGAGAAGCTGAACCTGCACAGCGCCGAC
Repeat 23 (encoding 123 aa)
Human sequence without codon alterations:
(SEQ ID NO: 189)
GCCAGTTCTGACCAGTGGAAGCGTCTGCACCTTTCTCTGCAGGAACTTCTGGTGTGGCTACAGCTGAAAGATGAT
GAATTAAGCCGGCAGGCACCTATTGGAGGCGACTTTCCAGCAGTTCAGAAGCAGAACGATGTACATAGGGCCTTC
AAGAGGGAATTGAAAACTAAAGAACCTGTAATCATGAGTACTCTTGAGACTGTACGAATATTTCTGACAGAGCAG
CCTTTGGAAGGACTAGAGAAACTCTACCAGGAGCCCAGAGAGCTGCCTCCTGAGGAGAGAGCCCAGAATGTCACT
CGGCTTCTACGAAAGCAGGCTGAGGAGGTCAATACTGAGTGGGAAAAATTGAACCTGCACTCCGCTGAC
Human sequence with codon optimization:
(SEQ ID NO: 190)
GCCAGCAGCGACCAGTGGAAGCGGCTGCACCTGAGCCTGCAGGAGCTGCTGGTGTGGCTGCAGCTGAAGGACGAC
GAGCTGAGCCGGCAGGCCCCCATCGGCGGCGACTTCCCCGCCGTGCAGAAGCAGAACGACGTGCACCGGGCCTTC
AAGCGGGAGCTGAAGACCAAGGAGCCCGTGATCATGAGCACCCTGGAGACCGTGCGGATCTTCCTGACCGAGCAG
CCCCTGGAGGGCCTGGAGAAGCTGTACCAGGAGCCCCGGGAGCTGCCCCCCGAGGAGCGGGCCCAGAACGTGACC
CGGCTGCTGCGGAAGCAGGCCGAGGAGGTGAACACCGAGTGGGAGAAGCTGAACCTGCACAGCGCCGAC
Repeat 24
Human Sequences without codon alterations:
(SEQ ID NO: 66)
TGGCAGAGAAAAATAGATGAGACCCTTGAAAGACTCCGGGAACTTCAAGAGGCCACGGATGAGCTGGACCTCAAG
CTGCGCCAAGCTGAGGTGATCAAGGGATCCTGGCAGCCCGTGGGCGATCTCCTCATTGACTCTCTCCAAGATCAC
CTCGAGAAAGTCAAGGCACTTCGAGGAGAAATTGCGCCTCTGAAAGAGAACGTGAGCCACGTCAATGACCTTGCT
CGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTT
Human Sequences with codon optimization:
(SEQ ID NO: 67)
TGGCAGCGGAAGATCGACGAGACCCTGGAGCGGCTGCAGGAGCTGCAGGAGGCCACCGACGAGCTGGACCTGAAG
CTGCGGCAGGCCGAGGTGATCAAGGGCAGCTGGCAGCCCGTGGGCGACCTGCTGATCGACAGCCTGCAGGACCAC
CTGGAGAAGGTGAAGGCCCTGCGGGGCGAGATCGCCCCCCTGAAGGAGAACGTGAGCCACGTGAACGACCTGGCC
CGGCAGCTGACCACCCTGGGCATCCAGCTGAGCCCCTACAACCTGAGCACCCTGGAGGACCTGAACACCCGGTGG
AAGCTGCTGCAGGTGGCCGTGGAGGACCGGGTGCGGCAGCTGCACGAGGCCCACCGGGACTTC
Hinge 4 + truncated C terminus
Human Sequences without codon alterations:
(SEQ ID NO: 68)
GGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTA
GCTGACCTGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAACAATTTGGTCAAC
GTCCCTCTCTGCGTGGATATGTGTCTGAACTGGCTGCTGAATGTTTATGATACGGGACGAACAGGGAGGATCCGT
GTCCTGTCTTTTAAAACTGGCATCATTTCCCTGTGTAAAGCACATTTGGAAGACAAGTACAGATACCTTTTCAAG
CAAGTGGCAAGTTCAACAGGATTTTGTGACCAGCGCAGGCTGGGCCTCCTTCTGCATGATTCTATCCAAATTCCA
AGACAGTTGGGTGAAGTTGCATCCTTTGGGGGCAGTAACATTGAGCCAAGTGTCCGGAGCTGCTTCCAATTTGCT
AATAATAAGCCAGAGATCGAAGCGGCCCTCTTCCTAGACTGGATGAGACTGGAACCCCAGTCCATGGTGTGGCTG
CCCGTCCTGCACAGAGTGGCTGCTGCAGAAACTGCCAAGCATCAGGCCAAATGTAACATCTGCAAAGAGTGTCCA
ATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGA
GACTTTGCCAAGGTACTAAAAAACAAATTTCGAACCAAAAGGTATTTTGCGAAGCATCCCCGAATGGGCTACCTG
CCAGTGCAGACTGTCTTAGAGGGGGACAACATGGAAACTCCC
Human Sequences with codon optimization:
(SEQ ID NO: 69)
GGCCCCGCCAGCCAGCACTTCCTGAGCACCAGCGTGCAGGGCCCCTGGGAGCGGGCCATCAGCCCCAACAAGGTG
CCCTACTACATCAACCACGAGACCCAGACCACCTGCTGGGACCACCCCAAGATGACCGAGCTGTACCAGAGCCTG
GCCGACCTGAACAACGTGCGGTTCAGCGCCTACCGGACCGCCATGAAGCTGCGGCGGCTGCAGAAGGCCCTGTGC
CTGGACCTGCTGAGCCTGAGCGCCGCCTGCGACGCCCTGGACCAGCACAACCTGAAGCAGAACGACCAGCCCATG
GACATCCTGCAGATCATCAACTGCCTGACCACCATCTACGACCGGCTGGAGCAGGAGCACAACAACCTGGTGAAC
GTGCCCCTGTGCGTGGACATGTGCCTGAACTGGCTGCTGAACGTGTACGACACCGGCCGGACCGGCCGGATCCGG
GTGCTGAGCTTCAAGACCGGCATCATCAGCCTGTGCAAGGCCCACCTGGAGGACAAGTACCGGTACCTGTTCAAG
CAGGTGGCCAGCAGCACCGGCTTCTGCGACCAGCGGCGGCTGGGCCTGCTGCTGCACGACAGCATCCAGATCCCC
CGGCAGCTGGGCGAGGTGGCCAGCTTCGGCGGCAGCAACATCGAGCCCAGCGTGCGGAGCTGCTTCCAGTTCGCC
AACAACAAGCCCGAGATCGAGGCCGCCCTGTTCCTGGACTGGATGCGGCTGGAGCCCCAGAGCATGGTGTGGCTG
CCCGTGCTGCACCGGGTGGCCGCCGCCGAGACCGCCAAGCACCAGGCCAAGTGCAACATCTGCAAGGAGTGCCCC
ATCATCGGCTTCCGGTACCGGAGCCTGAAGCACTTCAACTACGACATCTGCCAGAGCTGCTTCTTCAGCGGCCGG
GTGGCCAAGGGCCACAAGATGCACTACCCCATGGTGGAGTACTGCACCCCCACCACCAGCGGCGAGGACGTGCGG
GACTTCGCCAAGGTGCTGAAGAACAAGTTCCGGACCAAGCGGTACTTCGCCAAGCACCCCCGGATGGGCTACCTG
CCCGTGCAGACCGTGCTGGAGGGCGACAACATGGAGACCCCC
Syntrophin binding sites
Human Sequences without codon alterations:
(SEQ ID NO: 70)
GTTACTCTGATCAACTTCTGGCCAGTAGATTCTGCGCCTGCCTCGTCCCCTCAGCTTTCACACGATGATACTCAT
TCACGCATTGAACATTATGCTAGCAGGCTAGCAGAAATGGAAAACAGCAATGGATCTTATCTAAATGATAGCATC
TCTCCTAATGAGAGCATAGATGATGAACATTTGTTAATCCAGCATTACTGCCAAAGTTTGAACCAGGACTCCCCC
CTGAGCCAGCCTCGTAGT
Human Sequences with codon optimization:
(SEQ ID NO: 71)
GTGACCCTGATCAACTTCTGGCCCGTGGACAGCGCCCCCGCCAGCAGCCCCCAGCTGAGCCACGACGACACCCAC
AGCCGGATCGAGCACTACGCCAGCCGGCTGGCCGAGATGGAGAACAGCAACGGCAGCTACCTGAACGACAGCATC
AGCCCCAACGAGAGCATCGACGACGAGCACCTGCTGATCCAGCACTACTGCCAGAGCCTGAACCAGGACAGCCCC
CTGAGCCAGCCCCGGAGC
Coiled coil region
1st coiled coil plus proline rich region
Human sequence without codon alterations:
(SEQ ID NO: 72)
CCTGCCCAGATCTTGATTTCCTTAGAGAGTGAGGAAAGAGGGGAGCTAGAGAGAATCCTAGCAGATCTTGAGGAA
GAAAACAGGAATCTGCAAGCAGAATATGACCGTCTAAAGCAGCAGCACGAACATAAAGGCCTGTCCCCACTGCCG
TCCCCTCCTGAAATGATGCCCACCTCTCCCCAGAGTCCC
Human sequence with codon optimization:
(SEQ ID NO: 73)
CCCGCCCAGATCCTGATCAGCCTGGAGAGCGAGGAGCGGGGCGAGCTGGAGCGGATCCTGGCCGACCTGGAGGAG
GAGAACCGGAACCTGCAGGCCGAGTACGACCGGCTGAAGCAGCAGCACGAGCACAAGGGCCTGAGCCCCCTGCCC
AGCCCCCCCGAGATGATGCCCACCAGCCCCCAGAGCCCC
2nd coiled coil region to end of dystrophin
Human sequence without codon alterations:
(SEQ ID NO: 74)
CGGGATGCTGAGCTCATTGCTGAGGCCAAGCTACTGCGTCAACACAAAGGCCGCCTGGAAGCCAGGATGCAAATC
CTGGAAGACCACAATAAACAGCTGGAGTCACAGTTACACAGGCTAAGGCAGCTGCTGGAGCAACCCCAGGCAGAG
GCCAAAGTGAATGGCACAACGGTGTCCTCTCCTTCTACCTCTCTACAGAGGTCCGACAGCAGTCAGCCTATGCTG
CTCCGAGTGGTTGGCAGTCAAACTTCGGACTCCATGGGTGAGGAAGATCTTCTCAGTCCTCCCCAGGACACAAGC
ACAGGGTTAGAGGAGGTGATGGAGCAACTCAACAACTCCTTCCCTAGTTCAAGAGGAAGAAATACCCCTGGAAAG
CCAATGAGAGAGGACACAATG
Human sequence with codon optimization:
(SEQ ID NO: 75)
CGGGACGCCGAGCTGATCGCCGAGGCCAAGCTGCTGCGGCAGCACAAGGGCCGGCTGGAGGCCCGGATGCAGATC
CTGGAGGACCACAACAAGCAGCTGGAGAGCCAGCTGCACCGGCTGCGGCAGCTGCTGGAGCAGCCCCAGGCCGAG
GCCAAGGTGAACGGCACCACCGTGAGCAGCCCCAGCACCAGCCTGCAGCGGAGCGACAGCAGCCAGCCCATGCTG
CTGCGGGTGGTGGGCAGCCAGACCAGCGACAGCATGGGCGAGGAGGACCTGCTGAGCCCCCCCCAGGACACCAGC
ACCGGCCTGGAGGAGGTGATGGAGCAGCTGAACAACAGCTTCCCCAGCAGCCGGGGCCGGAACACCCCCGGCAAG
CCCATGCGGGAGGACACCATG

### Example 2. New Skeletal Muscle-Specific Promoter

The finding that the heart and skeletal muscle may need different micro-dystrophin constructs delivered to each muscle type, motivated the need for a small, skeletal muscle-specific promoter. Elements from the skeletal muscle alpha-actin promoter were taken to construct such a promoter. It consists of one or two copies of the 99 bp skeletal muscle alpha-actin enhancer and 274 bases of the core promoter elements, resulting in a 380 bp construct (or 479 bp with 2 enhancers).

Sequences of the promoter are provided below. In some embodiments, the promoter comprises any one of SEQ ID NOs: 76-80. In some embodiments, the promoter comprises one or more of SEQ ID NOs: 76-80.

380 bp construct
Enhancer
(Xbal site for cutting 5' end)
(SEQ ID NO: 76)
TCTAGAAAATCTGAAAGGCATAGCCCCATATATCAGTGATATAAATAGAA
CCTGCAGCAGGCTCTGGTAAATGATGACTACAAGGTGGACTGGGAGGCAG
CCCGGC
Core Promoter + KOZAK
(SEQ ID NO: 77)
AGGAGGGGCAAACCCGCTAGGGAGACACTCCATATACGGCCCGGCCCGCG
TTACCTGGGACCGGGCCAACCCGCTCCTTCTTTGGTCAACGCAGGGGACC
CGGGCGGGGGCCCAGGCCGCGAACCGGCCGAGGGAGGGGGCTCTAGTGCC
CAACACCCAAATATGGCTCGAGAAGGGCAGCGACATTCCTGCGGGGTGGC
GCGGAGGGAATGCCCGCGGGCTATATAAAACCTGAGCAGAGGGACAAGCG
GCCACCGCAGCGGACAGCGCCACC ATGG

ATG+G-initiation codon+G of coding sequence follows and completes the optimal KOZAK and creates a NcoI site (CCATGG) to cut the full promoter. The G after the ATG is not necessary if other cloning strategies are preferred.

The promoter sequence itself is 380 bp:
(SEQ ID NO: 78)
TCTAGAAAATCTGAAAGGCATAGCCCCATATATCAGTGATATAAATAGAA
CCTGCAGCAGGCTCTGGTAAATGATGACTACAAGGTGGACTGGGAGGCAG
CCCGGCAGGAGGGGCAAACCCGCTAGGGAGACACTCCATATACGGCCCGG
CCCGCGTTACCTGGGACCGGGCCAACCCGCTCCTTCTTTGGTCAACGCAG
GGGACCCGGGCGGGGGCCCAGGCCGCGAACCGGCCGAGGGAGGGGGCTCT
AGTGCCCAACACCCAAATATGGCTCGAGAAGGGCAGCGACATTCCTGCGG
GGTGGCGCGGAGGGAATGCCCGCGGGCTATATAAAACCTGAGCAGAGGGA
CAAGCGGCCACCGCAGCGGACAGCGCCACC
479 bp construct (stronger promoter)
Back to back Enhancers
(Xbal site for cutting 5' end)
(SEQ ID NO: 79)
TCTAGAAAATCTGAAAGGCATAGCCCCATATATCAGTGATATAAATAGAA
CCTGCAGCAGGCTCTGGTAAATGATGACTACAAGGTGGACTGGGAGGCAG
CCCGGCAATCTGAAAGGCATAGCCCCATATATCAGTGATATAAATAGAAC
CTGCAGCAGGCTCTGGTAAATGATGACTACAAGGTGGACTGGGAGGCAGC
CCGGC
Core Promoter + KOZAK
(SEQ ID NO: 77)
AGGAGGGGCAAACCCGCTAGGGAGACACTCCATATACGGCCCGGCCCGCG
TTACCTGGGACCGGGCCAACCCGCTCCTTCTTTGGTCAACGCAGGGGACC
CGGGCGGGGGCCCAGGCCGCGAACCGGCCGAGGGAGGGGGCTCTAGTGCC
CAACACCCAAATATGGCTCGAGAAGGGCAGCGACATTCCTGCGGGGTGGC
GCGGAGGGAATGCCCGCGGGCTATATAAAACCTGAGCAGAGGGACAAGCG
GCCACCGCAGCGGACAGCGCCACC ATGG
The promoter sequence itself is 479 bp:
(SEQ ID NO: 80)
TCTAGAAAATCTGAAAGGCATAGCCCCATATATCAGTGATATAAATAGAA
CCTGCAGCAGGCTCTGGTAAATGATGACTACAAGGTGGACTGGGAGGCAG
CCCGGCAATCTGAAAGGCATAGCCCCATATATCAGTGATATAAATAGAAC
CTGCAGCAGGCTCTGGTAAATGATGACTACAAGGTGGACTGGGAGGCAGC
CCGGCAGGAGGGGCAAACCCGCTAGGGAGACACTCCATATACGGCCCGGC
CCGCGTTACCTGGGACCGGGCCAACCCGCTCCTTCTTTGGTCAACGCAGG
GGACCCGGGCGGGGGCCCAGGCCGCGAACCGGCCGAGGGAGGGGGCTCTA
GTGCCCAACACCCAAATATGGCTCGAGAAGGGCAGCGACATTCCTGCGGG
GTGGCGCGGAGGGAATGCCCGCGGGCTATATAAAACCTGAGCAGAGGGAC
AAGCGGCCACCGCAGCGGACAGCGCCACC

### Example 3. New Approach for rAAV Delivery to Muscle

If both the heart and skeletal muscle are to be optimally targeted in rAAV gene delivery, then it would be best to deliver the cardiac rAAV and the skeletal muscle rAAV via the specific arterial beds for the muscles, rather than the intravenous delivery as is the common practice. This is accomplished using a drug delivery catheter that is introduced into the femoral artery and first advanced the heart. The cardiac rAAV is first delivered into the left and right coronary arteries. Following the cardiac rAAV delivery, the catheter is retracted to the aortic arch, where skeletal muscle-targeted rAAV is then delivered to the subclavian arteries and, if desired, the carotid arteries. The catheter is then retracted into the descending aorta, if desired, and the rest of the skeletal muscle rAAV is delivered via various descending aortic branches, depending on the desired skeletal muscle distribution.

To further enhance distribution of rAAV to skeletal muscle, a PDE5 inhibitor, such as sildenafil or tadalafil, is given 1 hour prior to rAAV delivery to increase blood flow to resting skeletal muscle. Additionally, inflation of a blood pressure cuff on each limb at a pressure below the systolic pressure and above the diastolyic pressure improves retention of virus in the limbs at the time of viral delivery.

### Example 4. Chimeric Utrophin/Dystrophin Micro-Dystrophin Constructs that are Optimized for Either Skeletal Muscle or Cardiac Muscle

There have been recent adverse events in the clinical trials using micro-dystrophins that are due to presentation of neoantigens in patients with deletions that include exons 3 to 11, which are present in all of the clinical trial micro-dystrophins. Two of the micro-dystrophins additionally present neoantigens that encompass exons 12-17. Since all DMD patients express utrophin and utrophin has two alternative actin binding N-termini following by three spectrin-like repeats that are similar to those of dystrophin, a chimeric micro-utrophin/dystrophin construct can be used in patients with N-terminal mutations in dystrophin, as well as other DMD patients as long as they are not deleted in any of the dystrophin regions present in the chimera.

Since the patients have mutations and deletions in different regions of dystrophin, there is the potential that for a given patient, a micro-dystrophin will present neoantigens, which leads to the possibility of an immune response against the expressed micro-dystrophin. With the exception of the extreme N-terminal and C-terminal mutations, it is possible to design micro-dystrophins that avoid neoantigen presentation for specific patient mutations. In the case of the N-terminal mutations, the use of the utrophin N-terminus will avoid neoantigen presentation and provide the needed actin binding. Thus, the micro-utrophin/dystrophin chimeras, and in particular any of constructs 14, 15, 16, and 17 listed in Table 6, can be used in almost all DMD patients without risk of a potential immune response.

In all cases, including the micro-dystrophins currently evaluated in clinical trials, the junctional peptide(s) created by splicing segments of utrophin or dystrophin to other segments of dystrophin is potentially a neoantigen and should be designed to minimize immunogenicity. There are two alternative utrophin N-terminal sequences expressed in mammals, which create different affinities for actin. Both can be utilized, but the form with the higher actin binding affinity is the form normally expressed in striated muscle and used in the sequences described in Tables 3-5. Examples of micro-dystrophins currently evaluated in clinical trials are PF-06939926 (Pfizer), SRP-9001-10 (Sarepta), and SGT-001 (Solid Bio).

Additionally, the loss of regulation of ion channels, including the TRPC1, 3, 6 channels, leads to residual pathology in skeletal and cardiac muscles that the micro-dystrophins currently in trials do not address. Accordingly, in some embodiments, regions in the C-terminus of dystrophin, including the syntrophin binding sites and the coiled coil regions that interact with dystrobrevin are included in the chimeric utrophin/dystrophin micro-dystrophin constructs if packaging size allows. The following chimeric utrophin/dystrophin micro-dystrophin constructs have been designed for AAV delivery that address the points described above.

To create the alternative utrophin N-terminus, the first 22 amino acids of the utrophin N-terminus are replaced with the alternative utrophin isoform sequence (27 amino acids) that weakens the actin binding:

(SEQ ID NO: 249)
MAKYGEHEASPDNGQNEFSDII →
(SEQ ID NO: 250)
MSGLAATTFHWKKCRLDLPGHVALQAC

TABLE 3
Chimeric Utrophin/Dystrophin Micro-dystrophin Constructs
that are Optimized for Cardiac Muscle for Patients
with N-terminal Mutations Prior to Exon 17

| | Size (amino |
|---|---|
| Elements included in construct | acid number) |
| 1) Nterm-R1 of Utrophin_Dystrophin R17-R19_R24 + | 1245 |
| H4 + trCterm | |
| 2) Nterm-R1 of utrophin_Dystrophin R17-R19_H4 to 1^{st} | 1389 |
| coiled coil and proline rich region that follows | |
| 3) Nterm-R1 of utrophin_Dystrophin R17-R19_R24 | 1326 |
| to end of syntrophin region | |

For patients with mutations after exon 13 but before exon 45, any of the micro-dystrophins disclosed herein can be used without the possibility of neoantigen presentation. This is because there is an alternative promoter that expresses the dystrophin-related protein, Dp140, that includes exon 45 and beyond. For patients with deletions after exon 43 and before exon 56, the Dp116 coding sequence includes all of repeat 23 and beyond.

While these patients can accept the dystrophin N-terminal region, a sequence of a chimeric utrophin/dystrophin micro-dystrophin that was developed for use in any patient with a mutation prior to exon 56 is shown below.

TABLE 4
Chimeric Utrophin/Dystrophin Micro-dystrophin Constructs
that are Optimized for Cardiac Muscle for Patients with
N-terminal Mutations After Exon 43 and Prior to Exon 56

| | Size (amino |
|---|---|
| Elements included in construct | acid number) |
| 4) Utrophin N-terminus through R1_R23 to end of | 1319 |
| dystrophin | |

TABLE 5
Chimeric Utrophin/Dystrophin Micro-dystrophin Constructs
that are Optimized for Skeletal Muscle for Patients
with Mutations/Deletions Before Exon 13

| | Size (amino |
|---|---|
| Elements included in construct | acid number) |
| 5) Utrophin Nterm-R2_Dystrophin R16-R17_R24 + | 1259 |
| H4 + trCterm | |
| 6) Utrophin Nterm-Dystrophin R2_R16-R17_H4 to | 1219 |
| end of syntrophin region | |
| 7) Utrophin Nterm-R2_Dystrophin R16-R17_H4 to 1^{st} | 1382 |
| coiled coil and proline rich region that follows | |
| 8) Utrophin Nterm-R1_Dystrophin R16-R17; R23 to | 1352 |
| end of syntrophin region | |
| 9) Utrophin Nterm-R2_Dystrophin R16-R17_R24 + | 1343 |
| H4 to end of syntrophin region | |
| 10) Utrophin Nterm-R2_Dystrophin R23 + R24 + | 1217 |
| H4 to end of syntrophin region | |
| 11) N-terminus through R2; R23 to end of 1st Coiled | 1264 |
| Coil | |
| 12) Utrophin Nterm-R3_Dystrophin R23 + R24 + | 1330 |
| H4 to end of syntrophin region | |
| 13) Utrophin N-terminus through R2; Dystrophin R23 | 1414 |
| to end | |

TABLE 6
Chimeric Utrophin/Dystrophin Micro-dystrophin Constructs
that are Optimized for Skeletal Muscle for Patients
with Mutations/Deletions Before Exon 59

| | Size (amino |
|---|---|
| Elements included in construct | acid number) |
| 14) Utrophin N-terminus through R3; Dystrophin R24 | 1403 |
| to end | |
| 15) Utrophin Nterm-R3_Dystrophin R24 + H4 to end | 1208 |
| of syntrophin region | |
| 16) Utrophin Nterm-R3_Dystrophin R24 + H4 to to | 1271 |
| 1st coiled coil and proline rich region that follows | |
| 17) Utrophin Nterm-H2_Dystrophin R24 + H4 to end | 1240 |
| of syntrophin region | |
| 18) Utrophin N-terminus through H2; Dystrophin R24 | 1435 |
| to end | |

TABLE 7
Chimeric Utrophin/Dystrophin Micro-dystrophin Constructs
that are Optimized for Skeletal Muscle for Patients
with Mutations/Deletions Before Exon 57

| | Size (amino |
|---|---|
| Elements included in construct | acid number) |
| 19) Utrophin N-terminus through R3; Dystrophin R23 | 1525 |
| to end | |
| 20) Utrophin Nterm-R3_Dystrophin R23 + H4 to to 1st | 1393 |
| coiled coil and proline rich region that follows | |

TABLE 8
Additional micro-dystrophins and chimeric utrophin/dystrophin
micro-dystrophins for use in both heart and skeletal muscle.

| | |
|---|---|
| | Size (amino |
| Elements included in construct | acid number) |

Micro-dystrophins

| | |
|---|---|
| 21) Nterm-R2_R17_H3_R22_H4-trCterm + SynBS | 1287 |
| 22) Nterm-R1_R17_H3_R21-R22_H4-trCterm + SynBS | 1287 |
| 23) Nterm-R2_R17_H3_R22_H4-trCterm + SynBS + coiled coil | 1350 |
| 24) Nterm-R1_R17_H3_R21-R22_H4-trCterm + SynBS + coiled coil | 1350 |
| 25) Nterm-R2_R17_R21-R22_H4-trCterm + SynBS | 1366 |

Micro-utrophin-dystrophin chimeras

| | |
|---|---|
| 26) UtroNterm-R2_DystroR17_H3_R22_H4-trCterm + SynBS | 1274 |
| 27) UtroNterm-R1_DystroR17_H3_R21-R22_H4-trCterm + SynBS | 1272 |
| 28) UtroNterm-R2_DystroR17_H3_R22_H4-trCterm + SynBS_coiled coil | 1337 |
| 29) UtroNterm-R1_DystroR17_H3_R21-R22_H4-trCterm + SynBS_coiled coil | 1335 |
| 30) UtroNterm-R2_DystroR17_R21-R22_H4-trCterm + SynBS | 1353 |

The amino acid sequences of modules to build the chimeric utrophin/dystrophin micro-dystrophin proteins described in this example are provided below as SEQ ID NOS: 191-213. The amino acid sequences of the micro-dystrophin constructs in Tables 3-8 are provided as SEQ ID NOs: 81-84, 93-101, and 149-165 elsewhere in the description. In some embodiments, the present disclosure provides chimeric utrophin/micro-dystrophin proteins comprising one or more amino acid sequences selected from the group consisting of SEQ ID NOs: 191-213.

In some aspects, the present disclosure provides cells and compositions comprising any of the disclosed chimeric proteins, recombinant nucleic acids, rAAV vectors, or rAAV particles. In some embodiments, the cell is a mammalian cell, such as a human cell. In some embodiments, disclosed herein are cells comprising chimeric proteins having an amino acid sequence comprising at least 90%, 95%, 98%, or 99% sequence identity to of any one of SEQ ID NOs: 81-84, 93-101, and 149-165. In particular embodiments, the cells comprise proteins encoded by a nucleic acid that comprises at least 80% sequence identity, such as 100% identity, to the nucleotide sequence of any one of SEQ ID NOs: 85-92. In some aspects, the present disclosure provides cells and compositions comprising recombinant nucleic acids or rAAV vectors comprising any of the nucleic acid sequences of SEQ ID NOs: 7-12, 22-39, 126-131, 133-148, 166-185. In some aspects, the present disclosure provides cells and compositions comprising recombinant nucleic acids or rAAV vectors comprising any of the nucleic acid sequences of SEQ ID NOs: 70, 72, and 207-247. In some aspects, the present disclosure provides cells and compositions comprising recombinant nucleic acids or rAAV vectors comprising any of the nucleic acid sequences comprising at least 80%, 90%, or greater than 90% identity to any of SEQ ID NOs: 7-12, 22-39, 85-92, 126-131, 133-148, 166-185; 70, 72, and 207-247.

In particular embodiments, cells, compositions, and rAAV particles that comprise an rAAV vector that comprises a nucleic acid sequence encoding a chimeric protein that contains a utrophin N-terminal domain ending at utrophin spectrin-like repeat 2 connected to a dystrophin C-terminal domain containing spectrin-like repeat 23 through the C-terminus.

### Additional Studies

The degeneration, or loss, of full-length dystrophin protein in DMD patients leaves the dystrophic muscle more susceptible to being damaged when contracting, which in turn causes breakdown of the muscle. Replacement micro-dystrophin gene therapies may be evaluated for their ability to protect the muscle against contraction-induced injury. This protection can be assessed by examining (i) the loss of creatine kinase (CK) from the muscle fibers due to membrane leakage and/or (ii) the loss of force generation due to muscle damage³. The metric of specific tension or specific force (SP₀), i.e. force per cross-sectional area, has been classically used to measure the strength of muscle fibers. Another metric is maximal absolute force generation. These experiments make use of eccentric, or lengthening, contractions to determine changes in muscle membrane integrity, which is deficient in Duchenne muscular dystrophy, and the mouse model for this disease (mdx).

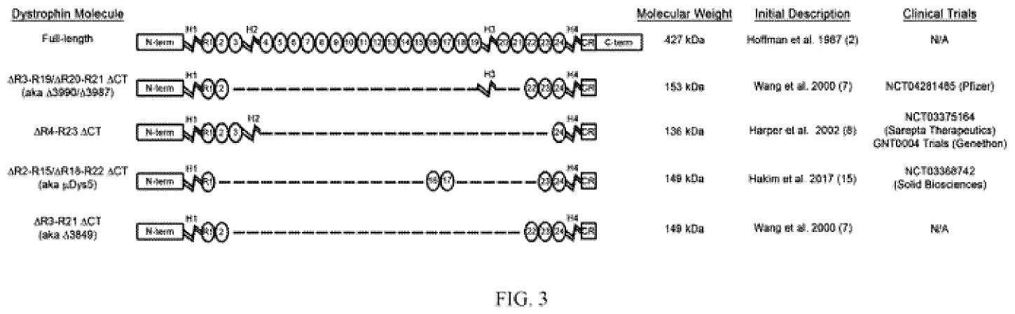

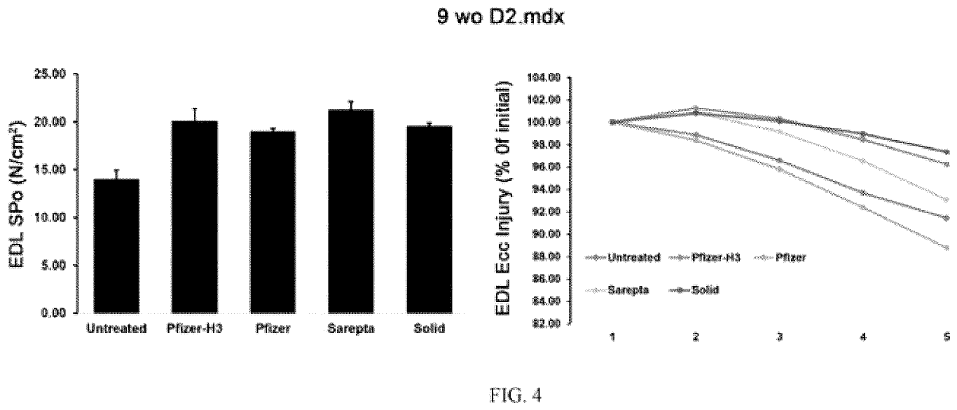

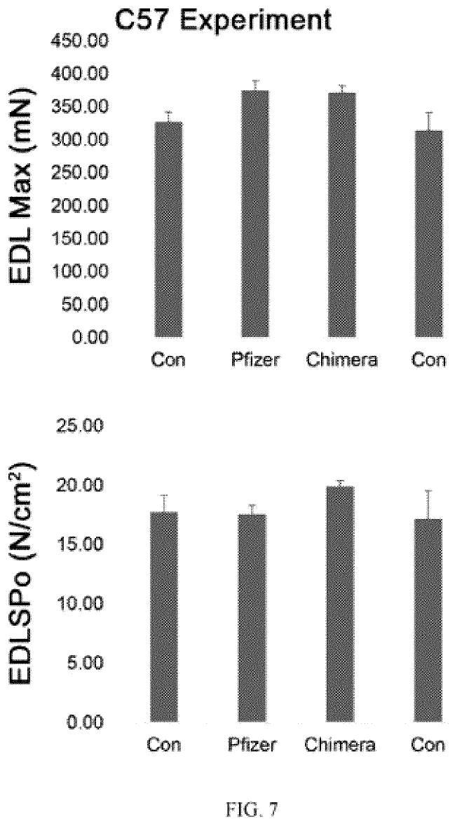

Herein, published protocols were used to compare the amount of protection conferred by an exemplary microdystrophin/utrophin chimera with those conferred by clinical micro-dystrophin constructs shown in the schematic of FIG. 3. These include PF-06939926 (Pfizer), SRP-9001-10 (Sarepta), and SGT-001 (Solid Bio). The chimera evaluated was the chimera that contains the N terminus of utrophin, ending at utrophin repeat 2, to dystrophin repeat 23, through the C-terminal syntrophin binding sites of dystrophin. These C-terminal syntrophin binding sites are missing in each of the clinical micro-dystrophin constructs. The results of these experiments are shown in FIGS. 4-7. The chimeric molecule was delivered to the skeletal muscle tissues of mice by AAV particles.

AAV particles were injected (via tail vein) at a dosage of 2×10¹⁴ vector genomes/kg into 5-week-old male D2.mdx mice (dystrophin-deficient mice in a DBA2/J genetic background). In parallel, D2.mdx. mice were also treated with three other micro-dystrophin constructs: PF-06939926, SRP-9001-10, and SGT-001. Subsets of mice were sacrificed at 9 weeks of age, or 16 weeks of age, to analyze the degree of skeletal muscle protection (skeletal muscle functional loss of function is apparent by this time).

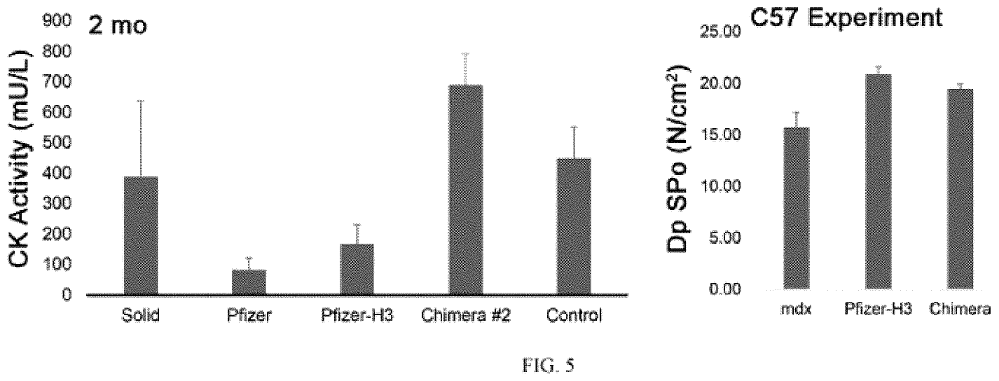

CK activity is assessed in FIG. 5 at 2 months following injections. The chimeric molecule provided the greatest protection against loss of CK from muscle fibers. Results further indicate improved maintenance of force production in diaphragm and extensor digitorum longus (EDL) muscles.

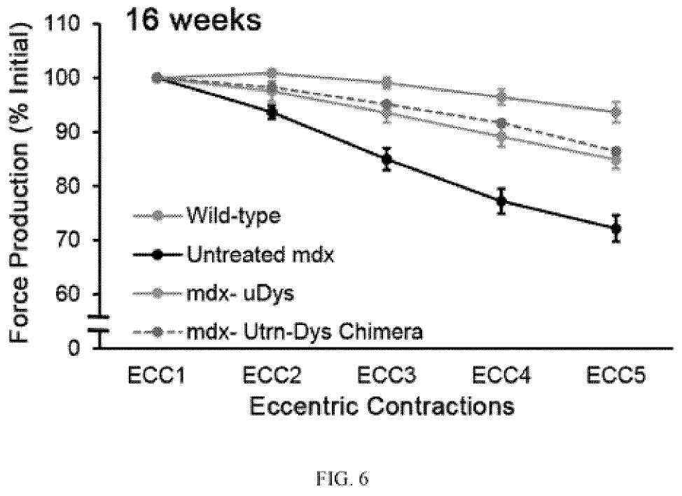

In particular, FIG. 6 shows the loss of force generation due to eccentric muscle damage. As shown in this figure, the chimera provides as much protection against contraction-induced injury as the micro-dystrophin construct, and protects more protection than control/null. AAV-delivery of either the micro-dystrophin or the micro-utrophin/dystrophin chimera may improve the force towards normal.

### REFERENCES CITED, EXAMPLE 4

Hoffman, E. P., Brown, R. H., Jr. & Kunkel, L. M. Dystrophin: the protein product of the Duchenne muscular dystrophy locus. Cell 51, 919-928 (1987).
Petrof, B. J., Shrager, J. B., Stedman, H. H., Kelly, A. M. & Sweeney, H. L. Dystrophin protects the sarcolemma from stresses developed during muscle contraction. Proc Natl Acad Sci USA 90, 3710-3714 (1993).
Wang, B., Li, J. & Xiao, X. Adeno-associated virus vector carrying human minidystrophin genes effectively ameliorates muscular dystrophy in mdx mouse model. Proc Natl Acad Sci USA 97, 13714-13719, doi: 10.1073/pnas.240335297 (2000).
Harper, S. Q. et al. Modular flexibility of dystrophin: implications for gene therapy of Duchenne muscular dystrophy. Nat Med 8, 253-261, doi: 10.1038/nm0302-253 (2002).
Hakim, C. H. et al. A Five-Repeat Micro-Dystrophin Gene Ameliorated Dystrophic Phenotype in the Severe DBA/2J-mdx Model of Duchenne Muscular Dystrophy. Mol Ther Methods Clin Dev 6, 216-230, doi: 10.1016/j.omtm.2017.06.006 (2017).

Listing of sequences for Example 4
Utrophin N-terminus (310 amino acids)
(SEQ ID NO: 191)
MAKYGEHEASPDNGQNEFSDIIKSRSDEHNDVQKKTFTKWINARFSKSGKPPINDMFTDLKDGRKLLDLLEGLTG
TSLPKERGSTRVHALNNVNRVLQVLHQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWDKVVKMSPIERLEHAFSKAQTYLGIE
KLLDPEDVAVQLPDKKSIIMYLTSLFEVLPQQVTIDAIREVETLPRKYKKECEEEAINIQSTAPEEEHESPRAET
PSTVTEVDMD
Utrophin Repeat 1 (122 amino acids)
(SEQ ID NO: 192)
LDSYQIALEEVLTWLLSAEDTFQEQDDISDDVEEVKDQFATHEAFMMELTAHQSSVGSVLQAGNQLITQGTLSDE
EEFEIQEQMTLLNARWEALRVESMDRQSRLHDVLM
Utrophin Repeat 2 (107 amino acids)
(SEQ ID NO: 193)
ELQKKQLQQLSAWLTLTEERI QKMETCPLDDDVKSLQKLLEEHKSLQSDLEAEQVKVNSLTHMVVIVDENSGESA
TAILEDQLQKLGERWTAVCRWTEERWNRLQEI
Utrophin Repeat 3 (111 amino acids)
(SEQ ID NO: 194)
NILWQELLEEQCLLKAWLTEKEEALNKVQTSNFKDQKELSVSVRRLAILKEDMEMKRQTLDQLSEI GQDVGQLLD
NSKASKKINSDSEELTQRWDSLVQRLEDSSNQVTQA
Utrophin Hinge 2 (32 amino acids)
(SEQ ID NO: 195)
VAKLGMSQIPQKDLLETVRVREQAITKKSKQE
Linker (not found in Utrophin or Dystrophin)
(SEQ ID NO: 196)
AASA
Dystrophin Repeat 16 (116 amino acids)
(SEQ ID NO: 197)
MMVMTEDMPLEISYVPSTYLTEITHVSQALLEVEQLLNAPDLCAKDFEDLFKQEESLKNIKDSLQQSSGRIDIIH
SKKTAALQSATPVERVKLQEALSQLDFQWEKVNKMYKDRQG
Dystrophin Repeat 17 (110 amino acids) in constructs 1-3 and 5-9
(SEQ ID NO: 198)
RSVEKWRRFHYDIKIFNQWLTEAEQFLRKTQIPENWEHAKYKWYLKELQDGIGQRQTVVRTLNATGEEII
QQSSKTDASILQEKLGSLNLRWQEVCKQLSDRKKRLEEQK
Dystrophin Repeat 18-Repeat 19 (214 amino acids)
(SEQ ID NO: 199)
NILSEFQRDLNEFVLWLEEADNIASIPLEPGKEQQLKEKLEQVKLLVEELPLRQGILKQLNETGGPVLVSAPISP
EEQDKLENKLKQTNLQWIKVSRALPEKQGEIEAQIKDLGQLEKKLEDLEEQLNHLLLWLSPIRNQLEIYNQPNQE
GPFDVKETEIAVQAKQPDVEEILSKGQHLYKEKPATQPVKRKLEDLSSEWKAVNRLLQELRAKQ
Dystrophin Repeat 23 (122 amino acids)
(SEQ ID NO: 200)
SSDQWKRLHLSLQELLVWLQLKDDELSRQAPIGGDFPAVQKQNDVHRAFKRELKTKEPVIMSTLETVRIFLTEQP
LEGLEKLYQEPRELPPEERAQNVTRLLRKQAEEVNTEWEKLNLHSAD
Dystrophin Repeat 24 (121 amino acids) in constructs 1, 3-5 and 8-13
(SEQ ID NO: 48)
WQRKIDETLERLQELQEATDELDLKLRQAEVIKGSWQPVGDLLIDSLQDHLEKVKALRGEIAPLKENVSHVNDLA
RQLTTLGIQLSPYNLSTLEDLNTRWKLLQVAVEDRVRQLHEAHRDF
Dystrophin Repeat 24 (120 amino acids) in constructs 14-20
(SEQ ID NO: 201)
QRKIDETLERLQELQEATDELDLKLRQAEVIKGSWQPVGDLLIDSLQDHLEKVKALRGEIAPLKENVSHVNDLAR
QLTTLGIQLSPYNLSTLEDLNTRWKLLQVAVEDRVRQLHEAHRDF
Dystrophin Hinge 4 + truncated C terminus (364 amino acids)
(SEQ ID NO: 202)
GPASQHFLSTSVQGPWERAISPNKVPYYINHETQTTCWDHPKMTELYQSLADLNNVRESAYRTAMKLRRLQKALC
LDLLSLSAACDALDQHNLKQNDQPMDILQIINCLTTIYDRLEQEHNNLVNVPLCVDMCLNWLLNVYDTGRTGRIR
VLSFKTGIISLCKAHLEDKYRYLFKQVASSTGFCDQRRLGLLLHDSIQIPRQLGEVASEGGSNIEPSVRSCFQFA
NNKPEIEAALFLDWMRLEPQSMVWLPVLHRVAAAETAKHQAKCNICKECPIIGFRYRSLKHENYDICQSCFFSGR
VAKGHKMHYPMVEYCTPTTSGEDVRDFAKVLKNKFRTKRYFAKHPRMGYLPVQTVLEGDNMETP
Syntrophin binding sites (81 amino acids)
(SEQ ID NO: 50)
VTLINFWPVDSAPASSPQLSHDDTHSRIEHYASRLAEMENSNGSYLNDSISPNESIDDEHLLIQHYCQSLNQDSP
LSQPRS
1st coiled coil adds 63aa; including proline rich region (63 amino acids)
(SEQ ID NO: 51)
PAQILISLESEERGELERILADLEEENRNLQAEYDRLKQQHEHKGLSPLPSPPEMMPTSPQSP
2nd coiled coil region to end of dystrophin (132 amino acids)
(SEQ ID NO: 52)
RDAELIAEAKLLRQHKGRLEARMQILEDHNKQLESQLHRLRQLLEQPQAEAKVNGTTVSSPSTSLQRSDSSQPML
LRVVGSQTSDSMGEEDLLSPPQDTSTGLEEVMEQLNNSFPSSRGRNTPGKPMREDTM
Nterm (325 amino acids)
(SEQ ID NO: 203)
MLWWEEVEDCYEREDVQKKTFTKWVNAQFSKFGKQHIENLFSDLQDGRRLLDLLEGLTGQKLPKEKGSTRVHALN
NVNKALRVLQNNNVDLVNIGSTDIVDGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAFNIARYQLGIEKLLDPEDVDTTYPDK
KSILMYITSLFQVLPQQVSIEAIQEVEMLPRPPKVTKEEHFQLHHQMHYSQQITVSLAQGYERTSSPKPRFKSYA
YTQAAYVTTSDPTRSPFPSQHLEAP
Repeat 1 (126 amino acids)
(SEQ ID NO: 41)
EDKSFGSSLMESEVNLDRYQTALEEVLSWLLSAEDTLQAQGEISNDVEVVKDQFHTHEGYMMDLTAHQGRVGNIL
QLGSKLIGTGKLSEDEETEVQEQMNLLNSRWECLRVASMEKQSNLHRVLMD
Repeat 2 (105 amino acids)
(SEQ ID NO: 42)
LQNQKLKELNDWLTKTEERTRKMEEEPLGPDLEDLKRQVQQHKVLQEDLEQEQVRVNSLTHMVVVVDESSGDHAT
AALEEQLKVLGDRWANICRWTEDRWVLLQD
Dystrophin R17 (113 amino acids) in constructs 21-30
(SEQ ID NO: 45)
RFDRSVEKWRRFHYDIKIFNQWLTEAEQFLRKTQIPENWEHAKYKWYLKELQDGIGQRQTVVRTLNATGEEIIQQ
SSKTDASILQEKLGSLNLRWQEVCKQLSDRKKRLEEQK
Hinge 3 (47 amino acids)
(SEQ ID NO: 204)
QPDLAPGLTTIGASPTQTVTLVTQPVVTKETAISKLEMPSSLMLEVP
Repeat 21 (105 amino acids)
(SEQ ID NO: 205)
KDSTQWLEAKEEAEQVLGQARAKLESWKEGPYTVDAIQKKITETKQLAKDLRQWQTNVDVANDLALKLLRDYSAD
DTRKVHMITENINASWRSIHKRVSEREAAL
Repeat 22 + linker (126 amino acids)
(SEQ ID NO: 206)
EETHRLLQQFPLDLEKFLAWLTEAETTANVLQDATRKERLLEDSKGVKELMKQWQDLQGEIEAHTDVYHNLDENS
QKILRSLEGSDDAVLLQRRLDNMNFKWSELRKKSLNIRSHLEASSDQWKRL

The nucleotide sequences of modules used to build the chimeric utrophin/dystrophin micro-dystrophin proteins described in this Example are provided below as SEQ ID NOs: 207-247. The nucleotide sequences of the micro-dystrophin constructs in Tables 3-8 are provided as SEQ ID Nos. 85-92, 102-119, 135-148 and 166-185 elsewhere in the description. In some embodiments, the present disclosure provides nucleic acid constructs comprising one or more of SEQ ID NOs: 70, 72, and 207-247. The present disclosure further provides nucleic acid constructs that comprise at least two, at least three, at least four, or at least five nucleotide sequences of any one of SEQ ID NOs: 70, 72, and 207-247. The present disclosure further provides nucleic acid constructs that comprise at least six, at least seven, at least eight, or more than eight nucleotide sequences of any one of SEQ ID NOs: 70, 72, and 207-247.

Utrophin N-terminus
Human Sequences without codon alterations:
(SEQ ID NO: 207)
ATGGCCAAGTATGGAGAACATGAAGCCAGTCCTGACAATGGGCAGAACGAATTCAGTGATATCATTAAGTCCAGA
TCTGATGAACACAATGACGTACAGAAGAAAACCTTTACCAAATGGATAAATGCTCGATTTTCAAAGAGTGGGAAA
CCACCCATCAATGATATGTTCACAGACCTCAAAGATGGAAGGAAGCTATTGGATCTTCTAGAAGGCCTCACAGGA
ACATCACTGCCAAAGGAACGTGGTTCCACAAGGGTACATGCCTTAAATAACGTCAACAGAGTGCTGCAGGTTTTA
CATCAGAACAATGTGGAATTAGTGAATATAGGGGGAACTGACATTGTGGATGGAAATCACAAACTGACTTTGGGG
TTACTTTGGAGCATCATTTTGCACTGGCAGGTGAAAGATGTCATGAAGGATGTCATGTCGGACCTGCAGCAGACG
AACAGTGAGAAGATCCTGCTCAGCTGGGTGCGTCAGACCACCAGGCCCTACAGCCAAGTCAACGTCCTCAACTTC
ACCACCAGCTGGACAGATGGACTCGCCTTTAATGCTGTCCTCCACCGACATAAACCTGATCTCTTCAGCTGGGAT
AAAGTTGTCAAAATGTCACCAATTGAGAGACTTGAACATGCCTTCAGCAAGGCTCAAACTTATTTGGGAATTGAA
AAGCTGTTAGATCCTGAAGATGTTGCCGTTCAGCTTCCTGACAAGAAATCCATAATTATGTATTTAACATCTTTG
TTTGAGGTGCTACCTCAGCAAGTCACCATAGACGCCATCCGTGAGGTAGAGACACTCCCAAGGAAATATAAAAAA
GAATGTGAAGAAGAGGCAATTAATATACAGAGTACAGCGCCTGAGGAGGAGCATGAGAGTCCCCGAGCTGAAACT
CCCAGCACTGTCACTGAGGTTGACATGGAT
Human Sequences with codon optimization:
(SEQ ID NO: 208)
ATGGCCAAGTACGGCGAGCACGAGGCCAGCCCCGACAACGGCCAGAACGAGTTCAGCGACATCATCAAGAGCCGG
AGCGACGAGCACAACGACGTGCAGAAGAAGACCTTCACCAAGTGGATCAACGCCCGGTTCAGCAAGAGCGGCAAG
CCCCCCATCAACGACATGTTCACCGACCTGAAGGACGGCCGGAAGCTGCTGGACCTGCTGGAGGGCCTGACCGGC
ACCAGCCTGCCCAAGGAGCGGGGCAGCACCCGGGTGCACGCCCTGAACAACGTGAACCGGGTGCTGCAGGTGCTG
CACCAGAACAACGTGGAGCTGGTGAACATCGGCGGCACCGACATCGTGGACGGCAACCACAAGCTGACCCTGGGC
CTGCTGTGGAGCATCATCCTGCACTGGCAGGTGAAGGACGTGATGAAGGACGTGATGAGCGACCTGCAGCAGACC
AACAGCGAGAAGATCCTGCTGAGCTGGGTGCGGCAGACCACCCGGCCCTACAGCCAGGTGAACGTGCTGAACTTC
ACCACCAGCTGGACCGACGGCCTGGCCTTCAACGCCGTGCTGCACCGGCACAAGCCCGACCTGTTCAGCTGGGAC
AAGGTGGTGAAGATGAGCCCCATCGAGCGGCTGGAGCACGCCTTCAGCAAGGCCCAGACCTACCTGGGCATCGAG
AAGCTGCTGGACCCCGAGGACGTGGCCGTGCAGCTGCCCGACAAGAAGAGCATCATCATGTACCTGACCAGCCTG
TTCGAGGTGCTGCCCCAGCAGGTGACCATCGACGCCATCCGGGAGGTGGAGACCCTGCCCCGGAAGTACAAGAAG
GAGTGCGAGGAGGAGGCCATCAACATCCAGAGCACCGCCCCCGAGGAGGAGCACGAGAGCCCCCGGGCCGAGACC
CCCAGCACCGTGACCGAGGTGGACATGGAC
Utrophin Repeat 1
Human Sequences without codon alterations:
(SEQ ID NO: 209)
CTGGACAGCTATCAGATTGCGTTGGAGGAAGTGCTGACCTGGTTGCTTTCTGCTGAGGACACTTTCCAGGAGCAG
GATGATATTTCTGATGATGTTGAAGAAGTCAAAGACCAGTTTGCAACCCATGAAGCTTTTATGATGGAACTGACT
GCACACCAGAGCAGTGTGGGCAGCGTCCTGCAGGCAGGCAACCAACTGATAACACAAGGAACTCTGTCAGACGAA
GAAGAATTTGAGATTCAGGAACAGATGACCCTGCTGAATGCTAGATGGGAGGCTCTTAGGGTGGAGAGTATGGAC
AGACAGTCCCGGCTGCACGATGTGCTGATG
Human Sequences with codon optimization:
(SEQ ID NO: 210)
CTGGACAGCTACCAGATCGCCCTGGAGGAGGTGCTGACCTGGCTGCTGAGCGCCGAGGACACCTTCCAGGAGCAG
GACGACATCAGCGACGACGTGGAGGAGGTGAAGGACCAGTTCGCCACCCACGAGGCCTTCATGATGGAGCTGACC
GCCCACCAGAGCAGCGTGGGCAGCGTGCTGCAGGCCGGCAACCAGCTGATCACCCAGGGCACCCTGAGCGACGAG
GAGGAGTTCGAGATCCAGGAGCAGATGACCCTGCTGAACGCCCGGTGGGAGGCCCTGCGGGTGGAGAGCATGGAC
CGGCAGAGCCGGCTGCACGACGTGCTGATG
Utrophin Repeat 2
Human Sequences without codon alterations:
(SEQ ID NO: 211)
GAACTGCAGAAGAAGCAACTGCAGCAGCTCTCCGCCTGGTTAACACTCACAGAGGAGCGCATTCAGAAGATGGAA
ACTTGCCCCCTGGATGATGATGTAAAATCTCTACAAAAGCTGCTAGAAGAACATAAAAGTTTGCAAAGTGATCTT
GAGGCTGAACAGGTGAAAGTAAATTCACTAACTCACATGGTGGTCATTGTTGATGAAAACAGTGGTGAGAGTGCT
ACAGCTATCCTAGAAGACCAGTTACAGAAACTTGGTGAGCGCTGGACAGCAGTATGCCGTTGGACTGAAGAACGC
TGGAATAGGTTACAAGAAATC
Human Sequences with codon optimization:
(SEQ ID NO: 212)
GAGCTGCAGAAGAAGCAGCTGCAGCAGCTGAGCGCCTGGCTGACCCTGACCGAGGAGCGGATCCAGAAGATGGAG
ACCTGCCCCCTGGACGACGACGTGAAGAGCCTGCAGAAGCTGCTGGAGGAGCACAAGAGCCTGCAGAGCGACCTG
GAGGCCGAGCAGGTGAAGGTGAACAGCCTGACCCACATGGTGGTGATCGTGGACGAGAACAGCGGCGAGAGCGCC
ACCGCCATCCTGGAGGACCAGCTGCAGAAGCTGGGCGAGCGGTGGACCGCCGTGTGCCGGTGGACCGAGGAGCGG
TGGAACCGGCTGCAGGAGATC
Utrophin Repeat 3
Human Sequences without codon alterations:
(SEQ ID NO: 213)
AATATATTGTGGCAGGAATTATTGGAAGAACAGTGCTTGTTGAAAGCTTGGTTAACCGAAAAAGAAGAGGCTTTA
AATAAAGTCCAGACAAGCAACTTCAAAGACCAAAAGGAACTAAGTGTCAGTGTTCGACGTCTGGCTATTTTGAAG
GAAGACATGGAAATGAAGCGTCAAACATTGGATCAGCTGAGTGAGATTGGCCAGGATGTGGGACAATTACTTGAT
AATTCCAAGGCATCTAAGAAGATCAACAGTGACTCAGAGGAACTGACTCAAAGATGGGATTCTTTGGTTCAGAGA
CTAGAAGATTCCTCCAACCAGGTGACTCAGGCT
Human Sequences with codon optimization:
(SEQ ID NO: 214)
AACATCCTGTGGCAGGAGCTGCTGGAGGAGCAGTGCCTGCTGAAGGCCTGGCTGACCGAGAAGGAGGAGGCCCTG
AACAAGGTGCAGACCAGCAACTTCAAGGACCAGAAGGAGCTGAGCGTGAGCGTGCGGCGGCTGGCCATCCTGAAG
GAGGACATGGAGATGAAGCGGCAGACCCTGGACCAGCTGAGCGAGATCGGCCAGGACGTGGGCCAGCTGCTGGAC
AACAGCAAGGCCAGCAAGAAGATCAACAGCGACAGCGAGGAGCTGACCCAGCGGTGGGACAGCCTGGTGCAGCGG
CTGGAGGACAGCAGCAACCAGGTGACCCAGGCC
Utrophin Hinge 2
Human Sequences without codon alterations:
(SEQ ID NO: 215)
GTAGCAAAGCTGGGGATGTCTCAGATTCCTCAGAAGGACCTTTTGGAGACTGTTCGTGTAAGAGAACAAGCAATT
ACAAAAAAATCTAAGCAGGAA
Human Sequences with codon optimization:
(SEQ ID NO: 216)
GTGGCCAAGCTGGGCATGAGCCAGATCCCCCAGAAGGACCTGCTGGAGACCGTGCGGGTGCGGGAGCAGGCCATC
ACCAAGAAGAGCAAGCAGGAG
Linker (no native sequence in Utrophin or Dystrophin)
(SEQ ID NO: 217)
GCCGCTAGCGCC
Dystrophin Repeat 16
Human Sequences without codon alterations:
(SEQ ID NO: 218)
ATGATGGTGATGACTGAAGACATGCCTTTGGAAATTTCTTATGTGCCTTCTACTTATTTGACTGAAATCACTCAT
GTCTCACAAGCCCTATTAGAAGTGGAACAACTTCTCAATGCTCCTGACCTCTGTGCTAAGGACTTTGAAGATCTC
TTTAAGCAAGAGGAGTCTCTGAAGAATATAAAAGATAGTCTACAACAAAGCTCAGGTCGGATTGACATTATTCAT
AGCAAGAAGACAGCAGCATTGCAAAGTGCAACGCCTGTGGAAAGGGTGAAGCTACAGGAAGCTCTCTCCCAGCTT
GATTTCCAATGGGAAAAAGTTAACAAAATGTACAAGGACCGACAAGGG
Human Sequences with codon optimization:
(SEQ ID NO: 219)
ATGATGGTGATGACCGAGGACATGCCCCTGGAGATCAGCTACGTGCCCAGCACCTACCTGACCGAGATCACCCAC
GTGAGCCAGGCCCTGCTGGAGGTGGAGCAGCTGCTGAACGCCCCCGACCTGTGCGCCAAGGACTTCGAGGACCTG
TTCAAGCAGGAGGAGAGCCTGAAGAACATCAAGGACAGCCTGCAGCAGAGCAGCGGCCGGATCGACATCATCCAC
AGCAAGAAGACCGCCGCCCTGCAGAGCGCCACCCCCGTGGAGCGGGTGAAGCTGCAGGAGGCCCTGAGCCAGCTG
GACTTCCAGTGGGAGAAGGTGAACAAGATGTACAAGGACCGGCAGGGC
Dystrophin Repeat 17 (in constructs 5-9)
Human Sequences without codon alterations:
(SEQ ID NO: 220)
CGATTTGACAGATCTGTTGAGAAATGGCGGCGTTTTCATTATGATATAAAGATATTTAATCAGTGGCTAACAGAA
GCTGAACAGTTTCTCAGAAAGACACAAATTCCTGAGAATTGGGAACATGCTAAATACAAATGGTATCTTAAGGAA
CTCCAGGATGGCATTGGGCAGCGGCAAACTGTTGTCAGAACATTGAATGCAACTGGGGAAGAAATAATTCAGCAA
TCCTCAAAAACAGATGCCAGTATTCTACAGGAAAAATTGGGAAGCCTGAATCTGCGGTGGCAGGAGGTCTGCAAA
CAGCTGTCAGACAGAAAAAAGAGGCTAGAAGAACAAAAG
Human Sequences with codon optimization:
(SEQ ID NO: 221)
CGGTTCGACCGGAGCGTGGAGAAGTGGCGGCGGTTCCACTACGACATCAAGATCTTCAACCAGTGGCTGACCGAG
GCCGAGCAGTTCCTGCGGAAGACCCAGATCCCCGAGAACTGGGAGCACGCCAAGTACAAGTGGTACCTGAAGGAG
CTGCAGGACGGCATCGGCCAGCGGCAGACCGTGGTGCGGACCCTGAACGCCACCGGCGAGGAGATCATCCAGCAG
AGCAGCAAGACCGACGCCAGCATCCTGCAGGAGAAGCTGGGCAGCCTGAACCTGCGGTGGCAGGAGGTGTGCAAG
CAGCTGAGCGACCGGAAGAAGCGGCTGGAGGAGCAGAAG
Dystrophin Repeat 17-Repeat 19
Human Sequences without codon alterations:
(SEQ ID NO: 222)
AGATCTGTTGAGAAATGGCGGCGTTTTCATTATGATATAAAGATATTTAATCAGTGGCTAACAGAAGCTGAACAG
TTTCTCAGAAAGACACAAATTCCTGAGAATTGGGAACATGCTAAATACAAATGGTATCTTAAGGAACTCCAGGAT
GGCATTGGGCAGCGGCAAACTGTTGTCAGAACATTGAATGCAACTGGGGAAGAAATAATTCAGCAATCCTCAAAA
ACAGATGCCAGTATTCTACAGGAAAAATTGGGAAGCCTGAATCTGCGGTGGCAGGAGGTCTGCAAACAGCTGTCA
GACAGAAAAAAGAGGCTAGAAGAACAAAAGAATATCTTGTCAGAATTTCAAAGAGATTTAAATGAATTTGTTTTA
TGGTTGGAGGAAGCAGATAACATTGCTAGTATCCCACTTGAACCTGGAAAAGAGCAGCAACTAAAAGAAAAGCTT
GAGCAAGTCAAGTTACTGGTGGAAGAGTTGCCCCTGCGCCAGGGAATTCTCAAACAATTAAATGAAACTGGAGGA
CCCGTGCTTGTAAGTGCTCCCATAAGCCCAGAAGAGCAAGATAAACTTGAAAATAAGCTCAAGCAGACAAATCTC
CAGTGGATAAAGGTTTCCAGAGCTTTACCTGAGAAACAAGGAGAAATTGAAGCTCAAATAAAAGACCTTGGGCAG
CTTGAAAAAAAGCTTGAAGACCTTGAAGAGCAGTTAAATCATCTGCTGCTGTGGTTATCTCCTATTAGGAATCAG
TTGGAAATTTATAACCAACCAAACCAAGAAGGACCATTTGACGTTAAGGAAACTGAAATAGCAGTTCAAGCTAAA
CAACCGGATGTGGAAGAGATTTTGTCTAAAGGGCAGCATTTGTACAAGGAAAAACCAGCCACTCAGCCAGTGAAG
AGGAAGTTAGAAGATCTGAGCTCTGAGTGGAAGGCGGTAAACCGTTTACTTCAAGAGCTGAGGGCAAAGCAG
Human Sequences with codon optimization:
(SEQ ID NO: 223)
CGGAGCGTGGAGAAGTGGCGGCGGTTCCACTACGACATCAAGATCTTCAACCAGTGGCTGACCGAGGCCGAGCAG
TTCCTGCGGAAGACCCAGATCCCCGAGAACTGGGAGCACGCCAAGTACAAGTGGTACCTGAAGGAGCTGCAGGAC
GGCATCGGCCAGCGGCAGACCGTGGTGCGGACCCTGAACGCCACCGGCGAGGAGATCATCCAGCAGAGCAGCAAG
ACCGACGCCAGCATCCTGCAGGAGAAGCTGGGCAGCCTGAACCTGCGGTGGCAGGAGGTGTGCAAGCAGCTGAGC
GACCGGAAGAAGCGGCTGGAGGAGCAGAAGAACATCCTGAGCGAGTTCCAGCGGGACCTGAACGAGTTCGTGCTG
TGGCTGGAGGAGGCCGACAACATCGCCAGCATCCCCCTGGAGCCCGGCAAGGAGCAGCAGCTGAAGGAGAAGCTG
GAGCAGGTGAAGCTGCTGGTGGAGGAGCTGCCCCTGCGGCAGGGCATCCTGAAGCAGCTGAACGAGACCGGCGGC
CCCGTGCTGGTGAGCGCCCCCATCAGCCCCGAGGAGCAGGACAAGCTGGAGAACAAGCTGAAGCAGACCAACCTG
CAGTGGATCAAGGTGAGCCGGGCCCTGCCCGAGAAGCAGGGCGAGATCGAGGCCCAGATCAAGGACCTGGGCCAG
CTGGAGAAGAAGCTGGAGGACCTGGAGGAGCAGCTGAACCACCTGCTGCTGTGGCTGAGCCCCATCCGGAACCAG
CTGGAGATCTACAACCAGCCCAACCAGGAGGGCCCCTTCGACGTGAAGGAGACCGAGATCGCCGTGCAGGCCAAG
CAGCCCGACGTGGAGGAGATCCTGAGCAAGGGCCAGCACCTGTACAAGGAGAAGCCCGCCACCCAGCCCGTGAAG
CGGAAGCTGGAGGACCTGAGCAGCGAGTGGAAGGCCGTGAACCGGCTGCTGCAGGAGCTGCGGGCCAAGCAG
Dystrophin Repeat 23
Human Sequences without codon alterations:
(SEQ ID NO: 224)
AGTTCTGACCAGTGGAAGCGTCTGCACCTTTCTCTGCAGGAACTTCTGGTGTGGCTACAGCTGAAAGATGATGAA
TTAAGCCGGCAGGCACCTATTGGAGGCGACTTTCCAGCAGTTCAGAAGCAGAACGATGTACATAGGGCCTTCAAG
AGGGAATTGAAAACTAAAGAACCTGTAATCATGAGTACTCTTGAGACTGTACGAATATTTCTGACAGAGCAGCCT
TTGGAAGGACTAGAGAAACTCTACCAGGAGCCCAGAGAGCTGCCTCCTGAGGAGAGAGCCCAGAATGTCACTCGG
CTTCTACGAAAGCAGGCTGAGGAGGTCAATACTGAGTGGGAAAAATTGAACCTGCACTCCGCTGAC
Human Sequences with codon optimization:
(SEQ ID NO: 225)
AGCAGCGACCAGTGGAAGCGGCTGCACCTGAGCCTGCAGGAGCTGCTGGTGTGGCTGCAGCTGAAGGACGACGAG
CTGAGCCGGCAGGCCCCCATCGGCGGCGACTTCCCCGCCGTGCAGAAGCAGAACGACGTGCACCGGGCCTTCAAG
CGGGAGCTGAAGACCAAGGAGCCCGTGATCATGAGCACCCTGGAGACCGTGCGGATCTTCCTGACCGAGCAGCCC
CTGGAGGGCCTGGAGAAGCTGTACCAGGAGCCCCGGGAGCTGCCCCCCGAGGAGCGGGCCCAGAACGTGACCCGG
CTGCTGCGGAAGCAGGCCGAGGAGGTGAACACCGAGTGGGAGAAGCTGAACCTGCACAGCGCCGAC
Dystrophin Repeat 24
Human Sequences without codon alterations:
(SEQ ID NO: 226)
TGGCAGAGAAAAATAGATGAGACCCTTGAAAGACTCCGGGAACTTCAAGAGGCCACGGATGAGCTGGACCTCAAG
CTGCGCCAAGCTGAGGTGATCAAGGGATCCTGGCAGCCCGTGGGCGATCTCCTCATTGACTCTCTCCAAGATCAC
CTCGAGAAAGTCAAGGCACTTCGAGGAGAAATTGCGCCTCTGAAAGAGAACGTGAGCCACGTCAATGACCTTGCT
CGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTT
Human Sequences with codon optimization:
(SEQ ID NO: 227)
TGGCAGAGAAAAATAGATGAGACCCTTGAAAGACTCCGGGAACTTCAAGAGGCCACGGATGAGCTGGACCTCAAG
CTGCGCCAAGCTGAGGTGATCAAGGGATCCTGGCAGCCCGTGGGCGATCTCCTCATTGACTCTCTCCAAGATCAC
CTCGAGAAAGTCAAGGCACTTCGAGGAGAAATTGCGCCTCTGAAAGAGAACGTGAGCCACGTCAATGACCTTGCT
CGCCAGCTTACCACTTTGGGCATTCAGCTCTCACCGTATAACCTCAGCACTCTGGAAGACCTGAACACCAGATGG
AAGCTTCTGCAGGTGGCCGTCGAGGACCGAGTCAGGCAGCTGCATGAAGCCCACAGGGACTTT
Dystrophin Repeat 24 (in constructs 14-20)
Human Sequences without codon alterations:
(SEQ ID NO: 228)
CAGAGAAAAATAGATGAGACCCTTGAAAGACTCCGGGAACTTCAAGAGGCCACGGATGAGCTGGACCTCAAGCTG
CGCCAAGCTGAGGTGATCAAGGGATCCTGGCAGCCCGTGGGCGATCTCCTCATTGACTCTCTCCAAGATCACCTC
GAGAAAGTCAAGGCACTTCGAGGAGAAATTGCGCCTCTGAAAGAGAACGTGAGCCACGTCAATGACCTTGCTCGC
CAGCTTACCACTTTGGGCATTCAGCTCTCACCGTATAACCTCAGCACTCTGGAAGACCTGAACACCAGATGGAAG
CTTCTGCAGGTGGCCGTCGAGGACCGAGTCAGGCAGCTGCATGAAGCCCACAGGGACTTT
Human Sequences with codon optimization:
(SEQ ID NO: 229)
CAGAGAAAAATAGATGAGACCCTTGAAAGACTCCGGGAACTTCAAGAGGCCACGGATGAGCTGGACCTCAAGCTG
CGCCAAGCTGAGGTGATCAAGGGATCCTGGCAGCCCGTGGGCGATCTCCTCATTGACTCTCTCCAAGATCACCTC
GAGAAAGTCAAGGCACTTCGAGGAGAAATTGCGCCTCTGAAAGAGAACGTGAGCCACGTCAATGACCTTGCTCGC
CAGCTTACCACTTTGGGCATTCAGCTCTCACCGTATAACCTCAGCACTCTGGAAGACCTGAACACCAGATGGAAG
CTTCTGCAGGTGGCCGTCGAGGACCGAGTCAGGCAGCTGCATGAAGCCCACAGGGACTTT
Dystrophin Hinge 4 + truncated C terminus
Human Sequences without codon alterations:
(SEQ ID NO: 230)
GGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTA
GCTGACCTGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAACAATTTGGTCAAC
GTCCCTCTCTGCGTGGATATGTGTCTGAACTGGCTGCTGAATGTTTATGATACGGGACGAACAGGGAGGATCCGT
GTCCTGTCTTTTAAAACTGGCATCATTTCCCTGTGTAAAGCACATTTGGAAGACAAGTACAGATACCTTTTCAAG
CAAGTGGCAAGTTCAACAGGATTTTGTGACCAGCGCAGGCTGGGCCTCCTTCTGCATGATTCTATCCAAATTCCA
AGACAGTTGGGTGAAGTTGCATCCTTTGGGGGCAGTAACATTGAGCCAAGTGTCCGGAGCTGCTTCCAATTTGCT
AATAATAAGCCAGAGATCGAAGCGGCCCTCTTCCTAGACTGGATGAGACTGGAACCCCAGTCCATGGTGTGGCTG
CCCGTCCTGCACAGAGTGGCTGCTGCAGAAACTGCCAAGCATCAGGCCAAATGTAACATCTGCAAAGAGTGTCCA
ATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGA
GACTTTGCCAAGGTACTAAAAAACAAATTTCGAACCAAAAGGTATTTTGCGAAGCATCCCCGAATGGGCTACCTG
CCAGTGCAGACTGTCTTAGAGGGGGACAACATGGAAACTCCC
Human Sequences with codon optimization:
(SEQ ID NO: 231)
GGTCCAGCATCTCAGCACTTTCTTTCCACGTCTGTCCAGGGTCCCTGGGAGAGAGCCATCTCGCCAAACAAAGTG
CCCTACTATATCAACCACGAGACTCAAACAACTTGCTGGGACCATCCCAAAATGACAGAGCTCTACCAGTCTTTA
GCTGACCTGAATAATGTCAGATTCTCAGCTTATAGGACTGCCATGAAACTCCGAAGACTGCAGAAGGCCCTTTGC
TTGGATCTCTTGAGCCTGTCAGCTGCATGTGATGCCTTGGACCAGCACAACCTCAAGCAAAATGACCAGCCCATG
GATATCCTGCAGATTATTAATTGTTTGACCACTATTTATGACCGCCTGGAGCAAGAGCACAACAATTTGGTCAAC
GTCCCTCTCTGCGTGGATATGTGTCTGAACTGGCTGCTGAATGTTTATGATACGGGACGAACAGGGAGGATCCGT
GTCCTGTCTTTTAAAACTGGCATCATTTCCCTGTGTAAAGCACATTTGGAAGACAAGTACAGATACCTTTTCAAG
CAAGTGGCAAGTTCAACAGGATTTTGTGACCAGCGCAGGCTGGGCCTCCTTCTGCATGATTCTATCCAAATTCCA
AGACAGTTGGGTGAAGTTGCATCCTTTGGGGGCAGTAACATTGAGCCAAGTGTCCGGAGCTGCTTCCAATTTGCT
AATAATAAGCCAGAGATCGAAGCGGCCCTCTTCCTAGACTGGATGAGACTGGAACCCCAGTCCATGGTGTGGCTG
CCCGTCCTGCACAGAGTGGCTGCTGCAGAAACTGCCAAGCATCAGGCCAAATGTAACATCTGCAAAGAGTGTCCA
ATCATTGGATTCAGGTACAGGAGTCTAAAGCACTTTAATTATGACATCTGCCAAAGCTGCTTTTTTTCTGGTCGA
GTTGCAAAAGGCCATAAAATGCACTATCCCATGGTGGAATATTGCACTCCGACTACATCAGGAGAAGATGTTCGA
GACTTTGCCAAGGTACTAAAAAACAAATTTCGAACCAAAAGGTATTTTGCGAAGCATCCCCGAATGGGCTACCTG
CCAGTGCAGACTGTCTTAGAGGGGGACAACATGGAAACTCCC
Syntrophin binding sites
Human Sequences with/without codon alterations:
(SEQ ID NO: 70)
GTTACTCTGATCAACTTCTGGCCAGTAGATTCTGCGCCTGCCTCGTCCCCTCAGCTTTCACACGATGATACTCAT
TCACGCATTGAACATTATGCTAGCAGGCTAGCAGAAATGGAAAACAGCAATGGATCTTATCTAAATGATAGCATC
TCTCCTAATGAGAGCATAGATGATGAACATTTGTTAATCCAGCATTACTGCCAAAGTTTGAACCAGGACTCCCCC
CTGAGCCAGCCTCGTAGT
1st coiled coil region + proline rich region
Human Sequences with/without codon alterations:
(SEQ ID NO: 72)
CCTGCCCAGATCTTGATTTCCTTAGAGAGTGAGGAAAGAGGGGAGCTAGAGAGAATCCTAGCAGATCTTGAGGAA
GAAAACAGGAATCTGCAAGCAGAATATGACCGTCTAAAGCAGCAGCACGAACATAAAGGCCTGTCCCCACTGCCG
TCCCCTCCTGAAATGATGCCCACCTCTCCCCAGAGTCCC
2nd coiled coil region to end of dystrophin
Human Sequences without codon alterations:
(SEQ ID NO: 232)
CGGGATGCTGAGCTCATTGCTGAGGCCAAGCTACTGCGTCAACACAAAGGCCGCCTGGAAGCCAGGATGCAAATC
CTGGAAGACCACAATAAACAGCTGGAGTCACAGTTACACAGGCTAAGGCAGCTGCTGGAGCAACCCCAGGCAGAG
GCCAAAGTGAATGGCACAACGGTGTCCTCTCCTTCTACCTCTCTACAGAGGTCCGACAGCAGTCAGCCTATGCTG
CTCCGAGTGGTTGGCAGTCAAACTTCGGACTCCATGGGTGAGGAAGATCTTCTCAGTCCTCCCCAGGACACAAGC
ACAGGGTTAGAGGAGGTGATGGAGCAACTCAACAACTCCTTCCCTAGITCAAGAGGAAGAAATACCCCTGGAAAG
CCAATGAGAGAGGACACAATG
Human Sequences with codon optimization:
(SEQ ID NO: 233)
CGGGATGCTGAGCTCATTGCTGAGGCCAAGCTACTGCGTCAACACAAAGGCCGCCTGGAAGCCAGGATGCAAATC
CTGGAAGACCACAATAAACAGCTGGAGTCACAGTTACACAGGCTAAGGCAGCTGCTGGAGCAACCCCAGGCAGAG
GCCAAAGTGAATGGCACAACGGTGTCCTCTCCTTCTACCTCTCTACAGAGGTCCGACAGCAGTCAGCCTATGCTG
CTCCGAGTGGTTGGCAGTCAAACTTCGGACTCCATGGGTGAGGAAGATCTTCTCAGTCCTCCCCAGGACACAAGC
ACAGGGTTAGAGGAGGTGATGGAGCAACTCAACAACTCCTTCCCTAGTTCAAGAGGAAGAAATACCCCTGGAAAG
CCAATGAGAGAGGACACAATG
Nterm
Human Sequences without codon alterations:
(SEQ ID NO: 234)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGCCAGCAGTCAGCCACACAACGACTGGAACATGCATTC
AACATCGCCAGATATCAATTAGGCATAGAGAAACTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCGATTCAAGAGCTATGCC
TACACACAGGCTGCTTATGTCACCACCTCTGACCCTACACGGAGCCCATTTCCTTCACAGCATTTGGAAGCTCCT
Human Sequences with codon optimization:
(SEQ ID NO: 235)
ATGCTGTGGTGGGAGGAGGTGGAGGACTGCTACGAGCGGGAGGACGTGCAGAAGAAGACCTTCACCAAGTGGGTG
AACGCCCAGTTCAGCAAGTTCGGCAAGCAGCACATCGAGAACCTGTTCAGCGACCTGCAGGACGGCCGGCGGCTG
CTGGACCTGCTGGAGGGCCTGACCGGCCAGAAGCTGCCCAAGGAGAAGGGCAGCACCCGGGTGCACGCCCTGAAC
AACGTGAACAAGGCCCTGCGGGTGCTGCAGAACAACAACGTGGACCTGGTGAACATCGGCAGCACCGACATCGTG
GACGGCAACCACAAGCTGACCCTGGGCCTGATCTGGAACATCATCCTGCACTGGCAGGTGAAGAACGTGATGAAG
AACATCATGGCCGGCCTGCAGCAGACCAACAGCGAGAAGATCCTGCTGAGCTGGGTGCGGCAGAGCACCCGGAAC
TACCCCCAGGTGAACGTGATCAACTTCACCACCAGCTGGAGCGACGGCCTGGCCCTGAACGCCCTGATCCACAGC
CACCGGCCCGACCTGTTCGACTGGAACAGCGTGGTGTGCCAGCAGAGCGCCACCCAGCGGCTGGAGCACGCCTTC
AACATCGCCCGGTACCAGCTGGGCATCGAGAAGCTGCTGGACCCCGAGGACGTGGACACCACCTACCCCGACAAG
AAGAGCATCCTGATGTACATCACCAGCCTGTTCCAGGTGCTGCCCCAGCAGGTGAGCATCGAGGCCATCCAGGAG
GTGGAGATGCTGCCCCGGCCCCCCAAGGTGACCAAGGAGGAGCACTTCCAGCTGCACCACCAGATGCACTACAGC
CAGCAGATCACCGTGAGCCTGGCCCAGGGCTACGAGCGGACCAGCAGCCCCAAGCCCCGGTTCAAGAGCTACGCC
TACACCCAGGCCGCCTACGTGACCACCAGCGACCCCACCCGGAGCCCCTTCCCCAGCCAGCACCTGGAGGCCCCC
R1
Human Sequences without codon alterations:
(SEQ ID NO: 236)
GAAGACAAGTCATTTGGCAGTTCATTGATGGAGAGTGAAGTAAACCTGGACCGTTATCAAACAGCTTTAGAAGAA
GTATTATCGTGGCTTCTTTCTGCTGAGGACACATTGCAAGCACAAGGAGAGATTTCTAATGATGTGGAAGTGGTG
AAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAAT
CTCCTAAATTCAAGATGGGAATGCCTCAGGGTAGCTAGCATGGAAAAACAAAGCAATTTACATAGAGTTTTAATG
GAT
Human Sequences with codon optimization:
(SEQ ID NO: 237)
GAGGACAAGAGCTTCGGCAGCAGCCTGATGGAGAGCGAGGTGAACCTGGACCGGTACCAGACCGCCCTGGAGGAG
GTGCTGAGCTGGCTGCTGAGCGCCGAGGACACCCTGCAGGCCCAGGGCGAGATCAGCAACGACGTGGAGGTGGTG
AAGGACCAGTTCCACACCCACGAGGGCTACATGATGGACCTGACCGCCCACCAGGGCCGGGTGGGCAACATCCTG
CAGCTGGGCAGCAAGCTGATCGGCACCGGCAAGCTGAGCGAGGACGAGGAGACCGAGGTGCAGGAGCAGATGAAC
CTGCTGAACAGCCGGTGGGAGTGCCTGCGGGTGGCCAGCATGGAGAAGCAGAGCAACCTGCACCGGGTGCTGATG
GAC
R2
Human Sequences without codon alterations:
(SEQ ID NO: 238)
CTCCAGAATCAGAAACTGAAAGAGTTGAATGACTGGCTAACAAAAACAGAAGAAAGAACAAGGAAAATGGAGGAA
GAGCCTCTTGGACCTGATCTTGAAGACCTAAAACGCCAAGTACAACAACATAAGGTGCTTCAAGAAGATCTAGAA
CAAGAACAAGTCAGGGTCAATTCTCTCACTCACATGGTGGTGGTAGTTGATGAATCTAGTGGAGATCACGCAACT
GCTGCTTTGGAAGAACAACTTAAGGTATTGGGAGATCGATGGGCAAACATCTGTAGATGGACAGAAGACCGCTGG
GTTCTTTTACAAGAC
Human Sequences with codon optimization:
(SEQ ID NO: 239)
CTGCAGAACCAGAAGCTGAAGGAGCTGAACGACTGGCTGACCAAGACCGAGGAGCGGACCCGGAAGATGGAGGAG
GAGCCCCTGGGCCCCGACCTGGAGGACCTGAAGCGGCAGGTGCAGCAGCACAAGGTGCTGCAGGAGGACCTGGAG
CAGGAGCAGGTGCGGGTGAACAGCCTGACCCACATGGTGGTGGTGGTGGACGAGAGCAGCGGCGACCACGCCACC
GCCGCCCTGGAGGAGCAGCTGAAGGTGCTGGGCGACCGGTGGGCCAACATCTGCCGGTGGACCGAGGACCGGTGG
GTGCTGCTGCAGGAC
Dystrophin R17 (in constructs 21-30)
Human Sequences without codon alterations:
(SEQ ID NO: 240)
CGATTTGACAGATCTGTTGAGAAATGGCGGCGTTTTCATTATGATATAAAGATATTTAATCAGTGGCTAACAGAA
GCTGAACAGTTTCTCAGAAAGACACAAATTCCTGAGAATTGGGAACATGCTAAATACAAATGGTATCTTAAGGAA
CTCCAGGATGGCATTGGGCAGCGGCAAACTGTTGTCAGAACATTGAATGCAACTGGGGAAGAAATAATTCAGCAA
TCCTCAAAAACAGATGCCAGTATTCTACAGGAAAAATTGGGAAGCCTGAATCTGCGGTGGCAGGAGGTCTGCAAA
CAGCTGTCAGACAGAAAAAAGAGGCTAGAAGAACAAAAG
Human Sequences with codon optimization:
(SEQ ID NO: 241)
CGGTTCGACCGGAGCGTGGAGAAGTGGCGGCGGTTCCACTACGACATCAAGATCTTCAACCAGTGGCTGACCGAG
GCCGAGCAGTTCCTGCGGAAGACCCAGATCCCCGAGAACTGGGAGCACGCCAAGTACAAGTGGTACCTGAAGGAG
CTGCAGGACGGCATCGGCCAGCGGCAGACCGTGGTGCGGACCCTGAACGCCACCGGCGAGGAGATCATCCAGCAG
AGCAGCAAGACCGACGCCAGCATCCTGCAGGAGAAGCTGGGCAGCCTGAACCTGCGGTGGCAGGAGGTGTGCAAG
CAGCTGAGCGACCGGAAGAAGCGGCTGGAGGAGCAGAAG
Hinge 3
Human Sequences without codon alterations:
(SEQ ID NO: 242)
CAGCCTGACCTAGCTCCTGGACTGACCACTATTGGAGCCTCTCCTACTCAGACTGTTACTCTGGTGACACAACCT
GTGGTTACTAAGGAAACTGCCATCTCCAAACTAGAAATGCCATCTTCCTTGATGTTGGAGGTACCT
Human Sequences with codon optimization:
(SEQ ID NO: 243)
CAGCCCGACCTGGCCCCCGGCCTGACCACCATCGGCGCCAGCCCCACCCAGACCGTGACCCTGGTGACCCAGCCC
GTGGTGACCAAGGAGACCGCCATCAGCAAGCTGGAGATGCCCAGCAGCCTGATGCTGGAGGTGCCC
Repeat 21
Human Sequences without codon alterations:
(SEQ ID NO: 244)
AAGGATTCAACACAATGGCTGGAAGCTAAGGAAGAAGCTGAGCAGGTCTTAGGACAGGCCAGAGCCAAGCTTGAG
TCATGGAAGGAGGGTCCCTATACAGTAGATGCAATCCAAAAGAAAATCACAGAAACCAAGCAGTTGGCCAAAGAC
CTCCGCCAGTGGCAGACAAATGTAGATGTGGCAAATGACTTGGCCCTGAAACTTCTCCGGGATTATTCTGCAGAT
GATACCAGAAAAGTCCACATGATAACAGAGAATATCAATGCCTCTTGGAGAAGCATTCATAAAAGGGTGAGTGAG
CGAGAGGCTGCTTTG
Human Sequences with codon optimization:
(SEQ ID NO: 245)
AAGGACAGCACCCAGTGGCTGGAGGCCAAGGAGGAGGCCGAGCAGGTGCTGGGCCAGGCCCGGGCCAAGCTGGAG
AGCTGGAAGGAGGGCCCCTACACCGTGGACGCCATCCAGAAGAAGATCACCGAGACCAAGCAGCTGGCCAAGGAC
CTGCGGCAGTGGCAGACCAACGTGGACGTGGCCAACGACCTGGCCCTGAAGCTGCTGCGGGACTACAGCGCCGAC
GACACCCGGAAGGTGCACATGATCACCGAGAACATCAACGCCAGCTGGCGGAGCATCCACAAGCGGGTGAGCGAG
CGGGAGGCCGCCCTG
Repeat 22 + linker
Human Sequences without codon alterations:
(SEQ ID NO: 246)
GAAGAAACTCATAGATTACTGCAACAGTTCCCCCTGGACCTGGAAAAGTTTCTTGCCTGGCTTACAGAAGCTGAA
ACAACTGCCAATGTCCTACAGGATGCTACCCGTAAGGAAAGGCTCCTAGAAGACTCCAAGGGAGTAAAAGAGCTG
ATGAAACAATGGCAAGACCTCCAAGGTGAAATTGAAGCTCACACAGATGTTTATCACAACCTGGATGAAAACAGC
CAAAAAATCCTGAGATCCCTGGAAGGTTCCGATGATGCAGTCCTGTTACAAAGACGTTTGGATAACATGAACTTC
AAGTGGAGTGAACTTCGGAAAAAGTCTCTCAACATTAGGTCCCATTTGGAAGCCAGTTCTGACCAGTGGAAGCGT
CTG
Human Sequences with codon optimization:
(SEQ ID NO: 247)
GAGGAGACCCACCGGCTGCTGCAGCAGTTCCCCCTGGACCTGGAGAAGTTCCTGGCCTGGCTGACCGAGGCCGAG
ACCACCGCCAACGTGCTGCAGGACGCCACCCGGAAGGAGCGGCTGCTGGAGGACAGCAAGGGCGTGAAGGAGCTG
ATGAAGCAGTGGCAGGACCTGCAGGGCGAGATCGAGGCCCACACCGACGTGTACCACAACCTGGACGAGAACAGC
CAGAAGATCCTGCGGAGCCTGGAGGGCAGCGACGACGCCGTGCTGCTGCAGCGGCGGCTGGACAACATGAACTTC
AAGTGGAGCGAGCTGCGGAAGAAGAGCCTGAACATCCGGAGCCACCTGGAGGCCAGCAGCGACCAGTGGAAGCGG
CTG

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including.” “carrying.” “having.” “containing.” “involving.” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A recombinant nucleic acid comprising a nucleotide sequence encoding a micro-dystrophin protein comprising a utrophin N-terminus.

2. The recombinant nucleic acid of claim 1, wherein the micro-dystrophin protein comprises the region of utrophin from the N-terminus to spectrin-like repeat 1.

3. The recombinant nucleic acid of claim 1, wherein the micro-dystrophin protein comprises the region of utrophin from the N-terminus to spectrin-like repeat 2.

4. The recombinant nucleic acid of claim 1, wherein the micro-dystrophin protein comprises the region of utrophin from the N-terminus to spectrin-like repeat 3.

5. The recombinant nucleic acid of claim 1, wherein the micro-dystrophin protein comprises the region of utrophin from the N-terminus to hinge 2.

6. The recombinant nucleic acid of any one of claims 1-5, wherein the nucleic acid comprises a nucleotide sequence that is at least 80%, 85%, 90%, 95%, 98%, or 99% identical to any one of SEQ ID NOs: 7-12 and 22-39.

7. The recombinant nucleic acid of any one of claims 1-6, wherein the nucleic acid comprises the nucleotide sequence of any one of SEQ ID NOs: 7-12 and 22-39.

8. The recombinant nucleic acid of any one of claims 1-5, wherein the nucleic acid comprises a nucleotide sequence that is at least 80%, 85%, 90%, 95%, 98%, or 99% identical to any one of SEQ ID NOs: 85-92, 126-131, 133 and 134.

9. The recombinant nucleic acid of any one of claims 1-5 and 8, wherein the nucleic acid comprises the nucleotide sequence of any one of SEQ ID NOs: 85-92, 126-131, 133 and 134.

10. The recombinant nucleic acid of any one of claims 1-5, wherein the nucleic acid comprises a nucleotide sequence that is at least 80%, 85%, 90%, 95%, 98%, or 99% identical to any one of SEQ ID NOs: 135-148.

11. The recombinant nucleic acid of any one of claims 1-5 and 10, wherein the nucleic acid comprises the nucleotide sequence of any one of SEQ ID NOs: 135-148.

12. The recombinant nucleic acid of any one of claims 1-5, wherein the nucleic acid comprises a nucleotide sequence that is at least 80%, 85%, 90%, 95%, 98%, or 99% identical to any one of SEQ ID NOs: 166-185.

13. The recombinant nucleic acid of any one of claims 1-5 and 12, wherein the nucleic acid comprises the nucleotide sequence of any one of SEQ ID NOs: 166-185.

14. The recombinant nucleic acid of any preceding claim, wherein the nucleic acid comprises one or more nucleotide sequences of any one of SEQ ID NOs: 70, 72, and 207-247.

15. The recombinant nucleic acid of any preceding claim, wherein the nucleic acid comprises at least two, at least three, at least four, or at least five nucleotide sequences of any one of SEQ ID NOs: 70, 72, and 207-247.

16. The recombinant nucleic acid of any preceding claim comprising a promoter that is operably linked to the nucleotide sequence encoding the micro-dystrophin protein.

17. The recombinant nucleic acid of claim 16, wherein the promoter is a cardiac-specific promoter.

18. The recombinant nucleic acid of claim 17, wherein the cardiac-specific promoter is a cardiac troponin T (cTnT) promoter.

19. The recombinant nucleic acid of claim 16, wherein the promoter is a skeletal muscle-specific promoter.

20. The recombinant nucleic acid of claim 19, wherein the skeletal muscle-specific promoter is a skeletal muscle alpha-actin promoter.

21. The recombinant nucleic acid of claim 19 or 20, wherein the skeletal muscle-specific promoter comprises a nucleotide sequence that is at least 80% identical to SEQ ID NO: 78 or SEQ ID NO: 80.

22. The recombinant nucleic acid of any one of claims 19-21, wherein the skeletal muscle-specific promoter comprises the nucleotide sequence of SEQ ID NO: 78 or SEQ ID NO: 80.

23. The recombinant nucleic acid of any preceding claim, wherein the micro-dystrophin protein encoded by the nucleic acid comprises an amino acid sequence that is at least 80% identical to the amino acid sequence of any one of SEQ ID NOs: 1-6 and 13-21.

24. The recombinant nucleic acid of any preceding claim, wherein the micro-dystrophin protein encoded by the nucleic acid comprises the amino acid sequence of any one of SEQ ID NOs: 1-6 and 13-21.

25. A chimeric protein encoded by the recombinant nucleic acid of any one of claims 1-24.

26. A recombinant adeno-associated virus (rAAV) vector comprising the recombinant nucleic acid of any one of claims 1-24.

27. An rAAV particle comprising the rAAV vector of claim 26 encapsidated in an AAV capsid.

28. The rAAV particle of claim 27, wherein the AAV capsid comprises a capsid protein derived from AAV1, AAV2, AAV3, AAV6, AAV7, AAV8, AAVrh.74, AAVrh.10, AAV2/6 or AAV9 serotypes.

29. A composition comprising the rAAV particle of claim 27 or 28, or the rAAV vector of claim 26.

30. A method of treating a skeletal muscle disorder in a subject in need thereof, comprising administering to the subject the recombinant nucleic acid of any one of claims 1-24, the micro-dystrophin protein of claim 25, the rAAV particle of claim 27 or 28, or the composition of claim 29.

31. A method of treating Duchenne muscular dystrophy (DMD) in a subject in need thereof, comprising administering to the subject the recombinant nucleic acid of any one of claims 1-24, the micro-dystrophin protein of claim 25, the rAAV particle of claim 27 or 28, or the composition of claim 29.

32. A method of treating Duchenne muscular dystrophy (DMD) in a subject in need thereof, the method comprising delivering to the subject a first rAAV particle in accordance with claim 27 or 28 and a second rAAV particle in accordance with claim 27 or 28 to skeletal muscle.

33. The method of claim 32, wherein the rAAV vector of the first rAAV particle comprises a recombinant nucleic acid comprising a cardiac-specific promoter.

34. The method of claim 32, wherein the rAAV vector of the second rAAV particle comprises a recombinant nucleic acid comprising a skeletal-specific promoter.

35. The method of any one of claims 32-34, wherein the first rAAV particle comprises a capsid protein derived from AAV9, AAVrh.74, or AAVrh.10.

36. The method of any one of claims 32-34, wherein the second rAAV particle comprises a capsid protein derived from AAV8.

37. The method of any one of claims 32-36, wherein the first and second rAAV particles are delivered by the same route of administration.

38. The method of any one of claims 32-36, wherein the first and second rAAV particles are delivered by different routes of administration.

39. The method of any one of claims 32-37, wherein the first and second rAAV particles are delivered via one or more catheters.

40. The method of any one of claims 32-36 and 38, wherein the first and second rAAV particles are delivered via two or more catheters.

41. The method of claim 40 comprising:
(i) introducing a catheter into the femoral artery and advancing to the heart;
(ii) delivering the first rAAV particle into the left and right coronary arteries;
(iii) retracting the catheter to the aortic arch; and
(iv) delivering the second rAAV particle to the subclavian and/or carotid arteries.

42. The method of claim 41 further comprising (v) retracting the catheter into the descending aorta and delivering the second rAAV particle to skeletal muscle via descending aortic branches.

43. The method of any one of claims 32-42 further comprising administering a vasodilator to the subject prior to the delivery of the first and second rAAV particles.

44. The method of claim 43, wherein the vasodilator is a PDE5 inhibitor.

45. The method of claim 44, wherein the PDE5 inhibitor is sildenafil or tadalafil.

46. The method of any one of claims 43-45, wherein the vasodilator is administered 10 minutes, 20 minutes, 30 minutes, 40 minutes, 50 minutes, or 1 hour prior to the delivery of first rAAV particle and/or second rAAV particle.

47. The method of any one of claims 32-46, wherein a blood pressure cuff is inflated on each limb at the time of delivery of the first rAAV particle and/or the second rAAV particle.

48. The method of any one of claims 32-47, wherein the micro-dystrophin protein encoded by the vector of the first rAAV particle comprises: an amino-terminal actin-binding domain, hinge domain 1, spectrin-like repeat 1, spectrin-like repeat 17, spectrin-like repeat 18, and spectrin-like repeat 19, and wherein the micro-dystrophin protein does not contain spectrin-like repeat 2 and/or spectrin-like repeat 3 of dystrophin.

49. The method of claim 48, wherein the micro-dystrophin protein further comprises one or more of: spectrin-like repeat 24, hinge domain 4, and dystroglycan binding site of dystrophin.

50. The method of claim 48 or 49, wherein the micro-dystrophin protein further comprises one or more syntrophin binding domains of dystrophin.

51. The method of any one of claims 48-50, wherein the micro-dystrophin protein further comprises one or more coiled coil domains of dystrophin.

52. The method of any one of claims 48-51, wherein the spectrin-like repeat 1 of the micro-dystrophin protein is directly coupled to the spectrin-like repeat 17.

53. The method of any one of claims 48-52, wherein the spectrin-like repeat 19 of the micro-dystrophin protein is directly coupled to spectrin-like repeat 24.

54. The method of any one of claims 48-53, wherein the spectrin-like repeat 19 of the micro-dystrophin protein is directly coupled to hinge domain 4.

55. The method of any one of claims 32-54, wherein the micro-dystrophin protein encoded by the vector of the second rAAV particle comprises: an amino-terminal actin-binding domain, hinge domain 1, spectrin-like repeat 1, spectrin-like repeat 2, spectrin-like repeat 16, and spectrin-like repeat 17, and wherein the micro-dystrophin protein does not contain spectrin-like repeat 3 of dystrophin.

56. The method of claim 55, wherein the micro-dystrophin protein further comprises one or more of: spectrin-like repeat 23, spectrin-like repeat 24, hinge domain 4, and dystroglycan binding site of dystrophin.

57. The method of claim 55 or 56, wherein the micro-dystrophin protein further comprises one or more syntrophin binding domains of dystrophin.

58. The method of any one of claims 55-57, wherein the micro-dystrophin protein further comprises one or more coiled coil domains of dystrophin.

59. The method of any one of claims 55-58, wherein the spectrin-like repeat 2 of the micro-dystrophin protein is directly coupled to spectrin-like repeat 16.

60. The method of any one of claims 55-59, wherein the spectrin-like repeat 17 of the micro-dystrophin protein is directly coupled to spectrin-like repeat 24.

61. The method of any one of claims 55-60, wherein the spectrin-like repeat 17 of the micro-dystrophin protein is directly coupled to hinge domain 4.

62. The method of any one of 32-61, wherein the micro-dystrophin protein encoded by the vector of the first or second rAAV particle comprises the region of dystrophin from spectrin-like repeat 24 to the C-terminus.

63. The method of any one of claims 32-61, wherein the micro-dystrophin protein encoded by the vector of the first or second rAAV particle comprises the region of dystrophin from spectrin-like repeat 24 to the end of the proline rich region following the first coiled coil domain.

64. The method of any one of claims 32-61, wherein the micro-dystrophin protein encoded by the vector of the first or second rAAV particle comprises the region of dystrophin from hinge domain 4 to the C-terminus.

65. The method of any one of claims 32-61, wherein the micro-dystrophin protein encoded by the vector of the first or second rAAV particle comprises the region of dystrophin from hinge domain 4 to the end of the proline rich region following the first coiled coil domain.

66. The method of any one of claims 32-61, wherein the micro-dystrophin protein encoded by the vector of the first or second rAAV particle comprises the region of dystrophin from hinge domain 4 to the end of the second syntrophin binding domain.

67. A method of delivering the rAAV particle of claim 27 or 28 to a skeletal muscle in a subject, the method comprising delivering the rAAV particle via a catheter.

68. The method of claim 67 comprising delivering the rAAV particle via a catheter to the subclavian and/or carotid arteries.

69. The method of claim 67 or 68, wherein the catheter is first introduced into the femoral artery and advanced to the subclavian and/or carotid arteries.

70. The method of any one of claims 67-69, further comprising retracting the catheter into the descending aorta and delivering the rAAV particle to skeletal muscle via descending aortic branches.

71. The method of any one of claims 67-70, comprising administering a vasodilator.

72. The method of 71, wherein the vasodilator is a PDE5 inhibitor.

73. The method of claim 72, wherein the PDE5 inhibitor is sildenafil or tadalafil.

74. The method of any one of claims 71-73, wherein the vasodilator is administered 10 minutes, 20 minutes, 30 minutes, 40 minutes, 50 minutes, or 1 hour prior to the delivery of the rAAV particle.

75. The method of any one of claims 67-74 further comprising inflating a blood pressure cuff on each limb at the time of delivery of the rAAV particle.

76. The method of any one of claims 67-75, wherein the skeletal muscle is fast-twitch or slow-twitch.

77. A chimeric protein comprising an amino acid sequence having at least 90%, 95%, 98%, or 99% sequence identity to of any one of SEQ ID NOs: 81-84, 93-101 and 149-155.

78. A chimeric protein comprising the amino acid sequence of any one of SEQ ID NOs: 81-84, 93-101 and 149-155.

79. The chimeric protein of claim 77 or 78, wherein the protein comprises the amino acid sequence of any one of SEQ ID NOs: 96-101.

80. The chimeric protein of any one of claims 77-79, wherein the protein comprises the amino acid sequence of SEQ ID NO: 98 or 101.

81. A recombinant nucleic acid encoding the chimeric protein of any one of claims 77-80.

82. The recombinant nucleic acid of claim 81, wherein the nucleic acid comprises the nucleotide sequence of any one of SEQ ID NOs: 85-92.

83. A recombinant nucleic acid comprising a nucleotide sequence encoding a micro-dystrophin protein comprising a utrophin N-terminus, wherein the nucleic acid comprises a sequence having at least 85%, 90%, 95%, 98%, or 99% sequence identity to of any one of SEQ ID NOs 85-92.

84. An rAAV vector comprising the recombinant nucleic acid of any one of claims 81-83.

85. An rAAV particle comprising the rAAV vector of claim 84.

86. A cell comprising the chimeric protein of any one of claims 25 and 77-80, the rAAV vector of claim 26 or 84, or the rAAV particle of any one of claims 27, 28 and 85.

87. The cell of claim 86, wherein the cell is a mammalian cell.

88. The cell of claim 86 or 87, wherein the cell is a human cell.
